(19) United States
(12) Patent Application Publication
(10) Pub. No.: US 2026/0027224 A1
(43) Pub. Date: Jan. 29, 2026

(54) ANTIBODY-DRUG CONJUGATES TARGETING CDH3 AND PREPARATION METHODS AND USES THEREOF

(71) Applicant: Sichuan Kelun-Biotech Biopharmaceutical Co., LTD., Chengdu (CN)

(72) Inventors: Hu Long, Chengdu (CN); Qiang Tian, Chengdu (CN); Xiaoxi Yuan, Chengdu (CN); Xiao Liu, Chengdu (CN); Xiang Zhang, Chengdu (CN); Jia Xie, Chengdu (CN); Hongmei Song, Chengdu (CN); Miao Tan, Chengdu (CN); Xiangyang Tan, Chengdu (CN); Junyou Ge, Chengdu (CN)

(21) Appl. No.: 19/278,353

(22) Filed: Jul. 23, 2025

Related U.S. Application Data
(63) Continuation of application No. PCT/CN2025/109903, filed on Jul. 22, 2025.

(30) Foreign Application Priority Data
Jul. 23, 2024 (CN) 202410993495.6
Jul. 15, 2025 (CN) 202510972695.8

(51) Int. Cl. A61K 47/68; A61P 35/00; C07K 16/28

(52) U.S. Cl. CPC A61K 47/6849; A61K 47/6803; A61P 35/00; C07K 16/28; C07K 2317/24; C07K 2317/92

(57) ABSTRACT

The present disclosure relates to an antibody-drug conjugate (ADC) for treating CDH3-positive cancer. The ADCs have a CDH3 antibody and a drug-linker molecule conjugated to the antibody. The ADC of the present disclosure has a better drug-antibody conjugation ratio and a good, targeted killing effect on CDH3-positive cancer. Also provided is a preparation method of the antibody-drug conjugate and use thereof in the treatment of the CDH3-positive cancer.

### CROSS-REFERENCE TO RELATED APPLICATIONS

This application is a continuation of PCT Application No. PCT/CN2025/109903, filed Jul. 22, 2025, which claims the benefit of Chinese Application No. 202410993495.6, filed on Jul. 23, 2024, and Chinese Application No. 202510972695.8, filed on Jul. 15, 2025, the disclosure of each of which is incorporated herein by reference in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “IEC250062PCT-sequence listings.xml”, created on Jul. 18, 2025, and is 167,586 bytes in size.

### TECHNICAL FIELD

The present disclosure relates to antibody-drug conjugates for treating CDH3-positive cancer. Specifically, the antibody-drug conjugates include CDH3 antibodies and drug-linker molecules conjugated to the antibodies. The present disclosure further provides a preparation method of the antibody-drug conjugates and applications thereof in the treatment of CDH3-positive cancer.

### BACKGROUND

Cancer is one of the leading causes of death in contemporary people. As a disease caused by the malignant transformation of healthy cells, cancer results from genetic changes, such as chromosomal translocation and mutations in tumor suppressor genes and growth factor receptors, leading to the malignant proliferation of cells. Defective apoptosis or programmed cell death further promotes the malignant transformation of the cells leading to cancer. According to the latest assessment by the International Agency for Research on Cancer (IARC) under the World Health Organization (WHO), there were 19.29 million new cancer cases worldwide in 2020, including 10.06 million male cases and 9.23 million female cases. There were 9.96 million cancer deaths worldwide in 2020, including 5.53 million male cases and 4.43 million female cases. This century, cancers are predicted to outweigh cardiovascular diseases, becoming the leading cause for premature death in most countries.

Cadherin, which is a type of transmembrane-associated glycoprotein, belongs to a family of cell adhesion molecules, and mediates cell-cell adhesion in a calcium ion-dependent manner to allow cells to interact with the environment, thus playing a key role in the morphogenesis of various organs and helping cells communicate with other cells in aspects such as immune surveillance, exosmosis, transport, tumor metastasis, wound healing and tissue positioning. CDH3 (P-cadherin) is involved in several intracellular homeostatic processes for regulating embryonic development and maintaining adult tissue structures, and is very important for cell differentiation, cell shapes, cell polarity, growth and migration. CDH3 plays an important role in protecting a structural integrity of epithelial tissues.

CDH3 is expressed in several adult tissues, and is generally co-expressed with E-cadherin, such as the epidermal basal layer, breast, and prostate. CDH3 is highly expressed and is associated with poor tumor prognosis in a plurality of tumor tissues, such as breast, ovarian, prostate, endometrial, skin, stomach, pancreas, and colon tumors. PCA062, a CDH3-targeted ADC drug developed by Novartis, showed acceptable safety and preliminary efficacy in phase I clinical trials, but the development was terminated due to causes such as weak antibody endocytosis and toxin-related toxicity. Fujifilm developed an anti-CDH3 antibody-conjugated radionuclide drug 90Y FF-21101, which was observed to have preliminary efficacy in multiple solid tumors such as ovarian cancer, lung cancer, bile duct cancer and colorectal cancer in phase I clinical trials and had good safety. Based on the high expression of CDH3 in a plurality of tumors and the clinical demonstration of safety and effectiveness of targeted drugs, CDH3 is an ideal therapeutic target.

Antibody-drug conjugates (ADCs) consist of an antibody, a bioactive molecule and a linker. The bioactive molecule is covalently conjugated to the antibody by the linker; and the antibody (for example, a monoclonal antibody) can specifically recognize a specific target on the surface of a tumor cell, thereby guiding an ADC to the surface of a cancer cell and allowing the ADC to enter the cancer cell by endocytosis effect. The bioactive molecules are then released inside the cancer cells to achieve the function of killing the cancer cells while not damaging normal tissue cells. Developing an antibody with better targeting ability, hydrophilicity, and endocytosis activity for conjugating cytotoxic small molecules, and preparing an ADC drug with a larger therapeutic window will provide patients with better treatment options.

### SUMMARY

The present disclosure provides an antibody-drug conjugate comprising a structure shown in formula (I): Ab′-(M-L-E-D)_{x′}, wherein:
Ab′ is an antibody or an antigen-binding fragment thereof, which specifically binds to CDH3;
M is a linker connected to the antibody or the antigen-binding fragment thereof;
L is a moiety connecting the linker M and E;
E is a moiety connecting L and D;
D is a cytotoxic drug moiety; and
x′ is selected from integers of 1 to 20, in particular embodiments, the integer is selected from 1-16, 1-10, 1-8, 4-8, 4, or 8.

In some embodiments, the antibody or the antigen-binding fragment thereof comprises complementarity determining regions (CDRs) as follows: (a) CDR-H1, CDR-H2 and CDR-H3 amino acid sequences of a heavy chain variable region (VH) amino acid sequence set forth in SEQ ID NO: 3 or 1; and/or CDR-L1, CDR-L2 and CDR-L3 amino acid sequences of a light chain variable region (VL) amino acid sequence set forth in SEQ ID NO: 4 or 2; (b) CDR-H1, CDR-H2 and CDR-H3 amino acid sequences of a VH amino acid sequence set forth in SEQ ID NO: 7 or 5; and/or CDR-L1, CDR-L2 and CDR-L3 amino acid sequences of a VL amino acid sequence set forth in SEQ ID NO: 8 or 6; (c) CDR-H1, CDR-H2 and CDR-H3 amino acid sequences of a VH amino acid sequence set forth in SEQ ID NO: 11 or 9; and/or CDR-L1, CDR-L2 and CDR-L3 amino acid sequences of a VL amino acid sequence set forth in SEQ ID NO: 12 or 10; (d) CDR-H1, CDR-H2 and CDR-H3 amino acid sequences of a VH amino acid sequence set forth in SEQ ID NO: 15 or 13; and/or CDR-L1, CDR-L2 and CDR-L3 amino acid sequences of a VL amino acid sequence set forth in SEQ ID NO: 14 or 12; or (e) CDR-H1, CDR-H2 and CDR-H3 amino acid sequences of a VH, and/or CDR-L1, CDR-L2 and CDR-L3 amino acid sequences of a VL, wherein at least one CDR of the VH and/or VL comprises a mutation compared to the VH and/or the VL of any one of (a) to (d), and the mutation is a substitution, deletion or addition of one, two or several amino acids (for example, a substitution, deletion or addition of 1, 2, or 3 amino acids); in particular embodiments, the substitution is a conservative substitution.

In some embodiments, the antibody or the antigen-binding fragment thereof comprises: (a) CDR-H1, CDR-H2 and CDR-H3 amino acid sequences of the VH amino acid sequence set forth in SEQ ID NO: 3; and CDR-L1, CDR-L2 and CDR-L3 amino acid sequences of the VL amino acid sequence set forth in SEQ ID NO: 4; (b) CDR-H1, CDR-H2 and CDR-H3 amino acid sequences of the VH amino acid sequence set forth in SEQ ID NO: 1; and CDR-L1, CDR-L2 and CDR-L3 amino acid sequences of the VL amino acid sequence set forth in SEQ ID NO: 2; (c) CDR-H1, CDR-H2 and CDR-H3 amino acid sequences of the VH amino acid sequence set forth in SEQ ID NO: 7; and CDR-L1, CDR-L2 and CDR-L3 amino acid sequences of the VL amino acid sequence set forth in SEQ ID NO: 8; (d) CDR-H1, CDR-H2 and CDR-H3 amino acid sequences of the VH amino acid sequence set forth in SEQ ID NO: 5; and CDR-L1, CDR-L2 and CDR-L3 amino acid sequences of the VL amino acid sequence set forth in SEQ ID NO: 6; (e) CDR-H1, CDR-H2 and CDR-H3 amino acid sequences of the VH amino acid sequence set forth in SEQ ID NO: 11; and CDR-L1, CDR-L2 and CDR-L3 amino acid sequences of the VL amino acid sequence set forth in SEQ ID NO: 12; (f) CDR-H1, CDR-H2 and CDR-H3 amino acid sequences of the VH amino acid sequence set forth in SEQ ID NO: 9; and CDR-L1, CDR-L2 and CDR-L3 amino acid sequences of the VL amino acid sequence set forth in SEQ ID NO: 10; (g) CDR-H1, CDR-H2 and CDR-H3 amino acid sequences of the VH amino acid sequence set forth in SEQ ID NO: 15; and CDR-L1, CDR-L2 and CDR-L3 amino acid sequences of the VL amino acid sequence set forth in SEQ ID NO: 16; or (h) CDR-H1, CDR-H2 and CDR-H3 amino acid sequences of the VH amino acid sequence set forth in SEQ ID NO: 13; and CDR-L1, CDR-L2 and CDR-L3 amino acid sequences of the VL amino acid sequence set forth in SEQ ID NO: 14.

In some embodiments, the antibody or the antigen-binding fragment thereof comprises: (1) the following VH and/or VL, wherein the CDRs are defined according to the Chothia numbering system: (1a) a VH comprising CDR-H1 comprising the amino acid sequence of SEQ ID NO: 17 or a variant thereof; CDR-H2 comprising the amino acid sequence of SEQ ID NO: 27 or a variant thereof; and CDR-H3 comprising the amino acid sequence of SEQ ID NO: 32 or a variant thereof; and/or a VL comprising CDR-L1 comprising the amino acid sequence of SEQ ID NO: 35 or a variant thereof; CDR-L2 comprising the amino acid sequence of SEQ ID NO: 38 or a variant thereof; and CDR-L3 comprising the amino acid sequence of SEQ ID NO: 42 or a variant thereof; (1b) a VH comprising CDR-H1 comprising the amino acid sequence of SEQ ID NO: 17 or a variant thereof; CDR-H2 comprising the amino acid sequence of SEQ ID NO: 22 or a variant thereof; and CDR-H3 comprising the amino acid sequence of SEQ ID NO: 32 or a variant thereof; and/or a VL comprising CDR-L1 comprising the amino acid sequence of SEQ ID NO: 35 or a variant thereof; CDR-L2 comprising the amino acid sequence of SEQ ID NO: 38 or a variant thereof; and CDR-L3 comprising the amino acid sequence of SEQ ID NO: 42 or a variant thereof; (1c) a VH comprising CDR-H1 comprising the amino acid sequence of SEQ ID NO: 44 or a variant thereof; CDR-H2 comprising the amino acid sequence of SEQ ID NO: 49 or a variant thereof; and CDR-H3 comprising the amino acid sequence of SEQ ID NO: 55 or a variant thereof; and/or a VL comprising CDR-L1 comprising the amino acid sequence of SEQ ID NO: 58 or a variant thereof; CDR-L2 comprising the amino acid sequence of SEQ ID NO: 63 or a variant thereof; and CDR-L3 comprising the amino acid sequence of SEQ ID NO: 66 or a variant thereof; (1d) a VH comprising CDR-H1 comprising the amino acid sequence of SEQ ID NO: 44 or a variant thereof; CDR-H2 comprising the amino acid sequence of SEQ ID NO: 49 or a variant thereof; and CDR-H3 comprising the amino acid sequence of SEQ ID NO: 55 or a variant thereof; and/or a VL comprising CDR-L1 comprising the amino acid sequence of SEQ ID NO: 58 or a variant thereof; CDR-L2 comprising the amino acid sequence of SEQ ID NO: 61 or a variant thereof; and CDR-L3 comprising the amino acid sequence of SEQ ID NO: 66 or a variant thereof; (1e) a VH comprising CDR-H1 comprising the amino acid sequence of SEQ ID NO: 68 or a variant thereof; CDR-H2 comprising the amino acid sequence of SEQ ID NO: 73 or a variant thereof; and CDR-H3 comprising the amino acid sequence of SEQ ID NO: 80 or a variant thereof; and/or a VL comprising CDR-L1 comprising the amino acid sequence of SEQ ID NO: 84 or a variant thereof; CDR-L2 comprising the amino acid sequence of SEQ ID NO: 88 or a variant thereof; and CDR-L3 comprising the amino acid sequence of SEQ ID NO: 91 or a variant thereof; (1f) a VH comprising CDR-H1 comprising the amino acid sequence of SEQ ID NO: 68 or a variant thereof; CDR-H2 comprising the amino acid sequence of SEQ ID NO: 73 or a variant thereof; and CDR-H3 comprising the amino acid sequence of SEQ ID NO: 80 or a variant thereof; and/or a VL comprising CDR-L1 comprising the amino acid sequence of SEQ ID NO: 83 or a variant thereof; CDR-L2 comprising the amino acid sequence of SEQ ID NO: 87 or a variant thereof; and CDR-L3 comprising the amino acid sequence of SEQ ID NO: 91 or a variant thereof; (1g) a VH comprising CDR-H1 comprising the amino acid sequence of SEQ ID NO: 93 or a variant thereof; CDR-H2 comprising the amino acid sequence of SEQ ID NO: 103 or a variant thereof; and CDR-H3 comprising the amino acid sequence of SEQ ID NO: 108 or a variant thereof; and/or a VL comprising CDR-L1 comprising the amino acid sequence of SEQ ID NO: 114 or a variant thereof; CDR-L2 comprising the amino acid sequence of SEQ ID NO: 117 or a variant thereof; and CDR-L3 comprising the amino acid sequence of SEQ ID NO: 119 or a variant thereof; or (1h) a VH comprising CDR-H1 comprising the amino acid sequence of SEQ ID NO: 93 or a variant thereof; CDR-H2 comprising the amino acid sequence of SEQ ID NO: 98 or a variant thereof; and CDR-H3 comprising the amino acid sequence of SEQ ID NO: 108 or a variant thereof; and/or a VL comprising CDR-L1 comprising the amino acid sequence of SEQ ID NO: 111 or a variant thereof; CDR-L2 comprising the amino acid sequence of SEQ ID NO: 117 or a variant thereof; and CDR-L3 comprising the amino acid sequence of SEQ ID NO: 119 or a variant thereof; or (2) the following VH and/or VL, wherein the CDRs are defined according to the AbM numbering system: (2a) a VH comprising CDR-H1 comprising the amino acid sequence of SEQ ID NO: 18 or a variant thereof; CDR-H2 comprising the amino acid sequence of SEQ ID NO: 28 or a variant thereof; and CDR-H3 comprising the amino acid sequence of SEQ ID NO: 32 or a variant thereof; and/or a VL comprising CDR-L1 comprising the amino acid sequence of SEQ ID NO: 35 or a variant thereof; CDR-L2 comprising the amino acid sequence of SEQ ID NO: 38 or a variant thereof; and CDR-L3 comprising the amino acid sequence of SEQ ID NO: 42 or a variant thereof; (2b) a VH comprising CDR-H1 comprising the amino acid sequence of SEQ ID NO: 18 or a variant thereof; CDR-H2 comprising the amino acid sequence of SEQ ID NO: 23 or a variant thereof; and CDR-H3 comprising the amino acid sequence of SEQ ID NO: 32 or a variant thereof; and/or a VL comprising CDR-L1 comprising the amino acid sequence of SEQ ID NO: 35 or a variant thereof; CDR-L2 comprising the amino acid sequence of SEQ ID NO: 38 or a variant thereof; and CDR-L3 comprising the amino acid sequence of SEQ ID NO: 42 or a variant thereof; (2c) a VH comprising CDR-H1 comprising the amino acid sequence of SEQ ID NO: 45 or a variant thereof; CDR-H2 comprising the amino acid sequence of SEQ ID NO: 50 or a variant thereof; and CDR-H3 comprising the amino acid sequence of SEQ ID NO: 55 or a variant thereof; and/or a VL comprising CDR-L1 comprising the amino acid sequence of SEQ ID NO: 58 or a variant thereof; CDR-L2 comprising the amino acid sequence of SEQ ID NO: 63 or a variant thereof; and CDR-L3 comprising the amino acid sequence of SEQ ID NO: 66 or a variant thereof; (2d) a VH comprising CDR-H1 comprising the amino acid sequence of SEQ ID NO: 45 or a variant thereof; CDR-H2 comprising the amino acid sequence of SEQ ID NO: 50 or a variant thereof; and CDR-H3 comprising the amino acid sequence of SEQ ID NO: 55 or a variant thereof; and/or a VL comprising CDR-L1 comprising the amino acid sequence of SEQ ID NO: 58 or a variant thereof; CDR-L2 comprising the amino acid sequence of SEQ ID NO: 61 or a variant thereof; and CDR-L3 comprising the amino acid sequence of SEQ ID NO: 66 or a variant thereof; (2e) a VH comprising CDR-H1 comprising the amino acid sequence of SEQ ID NO: 69 or a variant thereof; CDR-H2 comprising the amino acid sequence of SEQ ID NO: 74 or a variant thereof; and CDR-H3 comprising the amino acid sequence of SEQ ID NO: 80 or a variant thereof; and/or a VL comprising CDR-L1 comprising the amino acid sequence of SEQ ID NO: 84 or a variant thereof; CDR-L2 comprising the amino acid sequence of SEQ ID NO: 88 or a variant thereof; and CDR-L3 comprising the amino acid sequence of SEQ ID NO: 91 or a variant thereof; (2f) a VH comprising CDR-H1 comprising the amino acid sequence of SEQ ID NO: 69 or a variant thereof; CDR-H2 comprising the amino acid sequence of SEQ ID NO: 74 or a variant thereof; and CDR-H3 comprising the amino acid sequence of SEQ ID NO: 80 or a variant thereof; and/or a VL comprising CDR-L1 comprising the amino acid sequence of SEQ ID NO: 83 or a variant thereof; CDR-L2 comprising the amino acid sequence of SEQ ID NO: 87 or a variant thereof; and CDR-L3 comprising the amino acid sequence of SEQ ID NO: 91 or a variant thereof; (2g) a VH comprising CDR-H1 comprising the amino acid sequence of SEQ ID NO: 94 or a variant thereof; CDR-H2 comprising the amino acid sequence of SEQ ID NO: 104 or a variant thereof; and CDR-H3 comprising the amino acid sequence of SEQ ID NO: 108 or a variant thereof; and/or a VL comprising CDR-L1 comprising the amino acid sequence of SEQ ID NO: 114 or a variant thereof; CDR-L2 comprising the amino acid sequence of SEQ ID NO: 117 or a variant thereof; and CDR-L3 comprising the amino acid sequence of SEQ ID NO: 119 or a variant thereof; or (2h) a VH comprising CDR-H1 comprising the amino acid sequence of SEQ ID NO: 94 or a variant thereof; CDR-H2 comprising the amino acid sequence of SEQ ID NO: 99 or a variant thereof; and CDR-H3 comprising the amino acid sequence of SEQ ID NO: 108 or a variant thereof; and/or a VL comprising CDR-L1 comprising the amino acid sequence of SEQ ID NO: 111 or a variant thereof; CDR-L2 comprising the amino acid sequence of SEQ ID NO: 117 or a variant thereof; and CDR-L3 comprising the amino acid sequence of SEQ ID NO: 119 or a variant thereof; or (3) the following VH and/or VL, wherein the CDRs are defined according to the Kabat numbering system: (3a) a VH comprising CDR-H1 comprising the amino acid sequence of SEQ ID NO: 19 or a variant thereof; CDR-H2 comprising the amino acid sequence of SEQ ID NO: 29 or a variant thereof; and CDR-H3 comprising the amino acid sequence of SEQ ID NO: 32 or a variant thereof; and/or a VL comprising CDR-L1 comprising the amino acid sequence of SEQ ID NO: 35 or a variant thereof; CDR-L2 comprising the amino acid sequence of SEQ ID NO: 38 or a variant thereof; and CDR-L3 comprising the amino acid sequence of SEQ ID NO: 42 or a variant thereof; (3b) a VH comprising CDR-H1 comprising the amino acid sequence of SEQ ID NO: 19 or a variant thereof; CDR-H2 comprising the amino acid sequence of SEQ ID NO: 24 or a variant thereof; and CDR-H3 comprising the amino acid sequence of SEQ ID NO: 32 or a variant thereof; and/or a VL comprising CDR-L1 comprising the amino acid sequence of SEQ ID NO: 35 or a variant thereof; CDR-L2 comprising the amino acid sequence of SEQ ID NO: 38 or a variant thereof; and CDR-L3 comprising the amino acid sequence of SEQ ID NO: 42 or a variant thereof; (3c) a VH comprising CDR-H1 comprising the amino acid sequence of SEQ ID NO: 46 or a variant thereof; CDR-H2 comprising the amino acid sequence of SEQ ID NO: 52 or a variant thereof; and CDR-H3 comprising the amino acid sequence of SEQ ID NO: 55 or a variant thereof; and/or a VL comprising CDR-L1 comprising the amino acid sequence of SEQ ID NO: 58 or a variant thereof; CDR-L2 comprising the amino acid sequence of SEQ ID NO: 63 or a variant thereof; and CDR-L3 comprising the amino acid sequence of SEQ ID NO: 66 or a variant thereof; (3d) a VH comprising CDR-H1 comprising the amino acid sequence of SEQ ID NO: 46 or a variant thereof; CDR-H2 comprising the amino acid sequence of SEQ ID NO: 51 or a variant thereof; and CDR-H3 comprising the amino acid sequence of SEQ ID NO: 55 or a variant thereof; and/or a VL comprising CDR-L1 comprising the amino acid sequence of SEQ ID NO: 58 or a variant thereof; CDR-L2 comprising the amino acid sequence of SEQ ID NO: 61 or a variant thereof; and CDR-L3 comprising the amino acid sequence of SEQ ID NO: 66 or a variant thereof; (3e) a VH comprising CDR-H1 comprising the amino acid sequence of SEQ ID NO: 70 or a variant thereof; CDR-H2 comprising the amino acid sequence of SEQ ID NO: 77 or a variant thereof; and CDR-H3 comprising the amino acid sequence of SEQ ID NO: 80 or a variant thereof; and/or a VL comprising CDR-L1 comprising the amino acid sequence of SEQ ID NO: 84 or a variant thereof; CDR-L2 comprising the amino acid sequence of SEQ ID NO: 88 or a variant thereof; and CDR-L3 comprising the amino acid sequence of SEQ ID NO: 91 or a variant thereof; (3f) a VH comprising CDR-H1 comprising the amino acid sequence of SEQ ID NO: 70 or a variant thereof; CDR-H2 comprising the amino acid sequence of SEQ ID NO: 75 or a variant thereof; and CDR-H3 comprising the amino acid sequence of SEQ ID NO: 80 or a variant thereof; and/or a VL comprising CDR-L1 comprising the amino acid sequence of SEQ ID NO: 83 or a variant thereof; CDR-L2 comprising the amino acid sequence of SEQ ID NO: 87 or a variant thereof; and CDR-L3 comprising the amino acid sequence of SEQ ID NO: 91 or a variant thereof; (3g) a VH comprising CDR-H1 comprising the amino acid sequence of SEQ ID NO: 95 or a variant thereof; CDR-H2 comprising the amino acid sequence of SEQ ID NO: 105 or a variant thereof; and CDR-H3 comprising the amino acid sequence of SEQ ID NO: 108 or a variant thereof; and/or a VL comprising CDR-L1 comprising the amino acid sequence of SEQ ID NO: 114 or a variant thereof; CDR-L2 comprising the amino acid sequence of SEQ ID NO: 117 or a variant thereof; and CDR-L3 comprising the amino acid sequence of SEQ ID NO: 119 or a variant thereof; or (3h) a VH comprising CDR-H1 comprising the amino acid sequence of SEQ ID NO: 95 or a variant thereof; CDR-H2 comprising the amino acid sequence of SEQ ID NO: 100 or a variant thereof; and CDR-H3 comprising the amino acid sequence of SEQ ID NO: 108 or a variant thereof; and/or a VL comprising CDR-L1 comprising the amino acid sequence of SEQ ID NO: 111 or a variant thereof; CDR-L2 comprising the amino acid sequence of SEQ ID NO: 117 or a variant thereof; and CDR-L3 comprising the amino acid sequence of SEQ ID NO: 119 or a variant thereof; or (4) the following VH and/or VL, wherein the CDRs are defined according to the Contact numbering system: (4a) a VH comprising CDR-H1 comprising the amino acid sequence of SEQ ID NO: 20 or a variant thereof; CDR-H2 comprising the amino acid sequence of SEQ ID NO: 30 or a variant thereof; and CDR-H3 comprising the amino acid sequence of SEQ ID NO: 33 or a variant thereof; and/or a VL comprising CDR-L1 comprising the amino acid sequence of SEQ ID NO: 36 or a variant thereof; CDR-L2 comprising the amino acid sequence of SEQ ID NO: 40 or a variant thereof; and CDR-L3 comprising the amino acid sequence of SEQ ID NO: 43 or a variant thereof; (4b) a VH comprising CDR-H1 comprising the amino acid sequence of SEQ ID NO: 20 or a variant thereof; CDR-H2 comprising the amino acid sequence of SEQ ID NO: 25 or a variant thereof; and CDR-H3 comprising the amino acid sequence of SEQ ID NO: 33 or a variant thereof; and/or a VL comprising CDR-L1 comprising the amino acid sequence of SEQ ID NO: 36 or a variant thereof; CDR-L2 comprising the amino acid sequence of SEQ ID NO: 39 or a variant thereof; and CDR-L3 comprising the amino acid sequence of SEQ ID NO: 43 or a variant thereof; (4c) a VH comprising CDR-H1 comprising the amino acid sequence of SEQ ID NO: 47 or a variant thereof; CDR-H2 comprising the amino acid sequence of SEQ ID NO: 53 or a variant thereof; and CDR-H3 comprising the amino acid sequence of SEQ ID NO: 56 or a variant thereof; and/or a VL comprising CDR-L1 comprising the amino acid sequence of SEQ ID NO: 59 or a variant thereof; CDR-L2 comprising the amino acid sequence of SEQ ID NO: 64 or a variant thereof; and CDR-L3 comprising the amino acid sequence of SEQ ID NO: 67 or a variant thereof; (4d) a VH comprising CDR-H1 comprising the amino acid sequence of SEQ ID NO: 47 or a variant thereof; CDR-H2 comprising the amino acid sequence of SEQ ID NO: 53 or a variant thereof; and CDR-H3 comprising the amino acid sequence of SEQ ID NO: 56 or a variant thereof; and/or a VL comprising CDR-L1 comprising the amino acid sequence of SEQ ID NO: 59 or a variant thereof; CDR-L2 comprising the amino acid sequence of SEQ ID NO: 62 or a variant thereof; and CDR-L3 comprising the amino acid sequence of SEQ ID NO: 67 or a variant thereof; (4e) a VH comprising CDR-H1 comprising the amino acid sequence of SEQ ID NO: 71 or a variant thereof; CDR-H2 comprising the amino acid sequence of SEQ ID NO: 78 or a variant thereof; and CDR-H3 comprising the amino acid sequence of SEQ ID NO: 81 or a variant thereof; and/or a VL comprising CDR-L1 comprising the amino acid sequence of SEQ ID NO: 85 or a variant thereof; CDR-L2 comprising the amino acid sequence of SEQ ID NO: 89 or a variant thereof; and CDR-L3 comprising the amino acid sequence of SEQ ID NO: 92 or a variant thereof; (4f) a VH comprising CDR-H1 comprising the amino acid sequence of SEQ ID NO: 71 or a variant thereof; CDR-H2 comprising the amino acid sequence of SEQ ID NO: 76 or a variant thereof; and CDR-H3 comprising the amino acid sequence of SEQ ID NO: 81 or a variant thereof; and/or a VL comprising CDR-L1 comprising the amino acid sequence of SEQ ID NO: 85 or a variant thereof; CDR-L2 comprising the amino acid sequence of SEQ ID NO: 89 or a variant thereof; and CDR-L3 comprising the amino acid sequence of SEQ ID NO: 92 or a variant thereof; (4g) a VH comprising CDR-H1 comprising the amino acid sequence of SEQ ID NO: 96 or a variant thereof; CDR-H2 comprising the amino acid sequence of SEQ ID NO: 106 or a variant thereof; and CDR-H3 comprising the amino acid sequence of SEQ ID NO: 109 or a variant thereof; and/or a VL comprising CDR-L1 comprising the amino acid sequence of SEQ ID NO: 115 or a variant thereof; CDR-L2 comprising the amino acid sequence of SEQ ID NO: 118 or a variant thereof; and CDR-L3 comprising the amino acid sequence of SEQ ID NO: 120 or a variant thereof; or (4h) a VH comprising CDR-H1 comprising the amino acid sequence of SEQ ID NO: 96 or a variant thereof; CDR-H2 comprising the amino acid sequence of SEQ ID NO: 101 or a variant thereof; and CDR-H3 comprising the amino acid sequence of SEQ ID NO: 109 or a variant thereof; and/or a VL comprising CDR-L1 comprising the amino acid sequence of SEQ ID NO: 112 or a variant thereof; CDR-L2 comprising the amino acid sequence of SEQ ID NO: 118 or a variant thereof; and CDR-L3 comprising the amino acid sequence of SEQ ID NO: 120 or a variant thereof; or (5) the following VH and/or VL, wherein the CDRs are defined according to the IMGT numbering system: (5a) a VH comprising CDR-H1 comprising the amino acid sequence of SEQ ID NO: 21 or a variant thereof; CDR-H2 comprising the amino acid sequence of SEQ ID NO: 31 or a variant thereof; and CDR-H3 comprising the amino acid sequence of SEQ ID NO: 34 or a variant thereof; and/or a VL comprising CDR-L1 comprising the amino acid sequence of SEQ ID NO: 37 or a variant thereof; CDR-L2 comprising the amino acid sequence of SEQ ID NO: 41 or a variant thereof; and CDR-L3 comprising the amino acid sequence of SEQ ID NO: 42 or a variant thereof; (5b) a VH comprising CDR-H1 comprising the amino acid sequence of SEQ ID NO: 21 or a variant thereof; CDR-H2 comprising the amino acid sequence of SEQ ID NO: 26 or a variant thereof; and CDR-H3 comprising the amino acid sequence of SEQ ID NO: 34 or a variant thereof; and/or a VL comprising CDR-L1 comprising the amino acid sequence of SEQ ID NO: 37 or a variant thereof; CDR-L2 comprising the amino acid sequence of SEQ ID NO: 41 or a variant thereof; and CDR-L3 comprising the amino acid sequence of SEQ ID NO: 42 or a variant thereof; (5c) a VH comprising CDR-H1 comprising the amino acid sequence of SEQ ID NO: 48 or a variant thereof; CDR-H2 comprising the amino acid sequence of SEQ ID NO: 54 or a variant thereof; and CDR-H3 comprising the amino acid sequence of SEQ ID NO: 57 or a variant thereof; and/or a VL comprising CDR-L1 comprising the amino acid sequence of SEQ ID NO: 60 or a variant thereof; CDR-L2 comprising the amino acid sequence of SEQ ID NO: 65 or a variant thereof; and CDR-L3 comprising the amino acid sequence of SEQ ID NO: 66 or a variant thereof; (5d) a VH comprising CDR-H1 comprising the amino acid sequence of SEQ ID NO: 72 or a variant thereof; CDR-H2 comprising the amino acid sequence of SEQ ID NO: 79 or a variant thereof; and CDR-H3 comprising the amino acid sequence of SEQ ID NO: 82 or a variant thereof; and/or a VL comprising CDR-L1 comprising the amino acid sequence of SEQ ID NO: 86 or a variant thereof; CDR-L2 comprising the amino acid sequence of SEQ ID NO: 90 or a variant thereof; and CDR-L3 comprising the amino acid sequence of SEQ ID NO: 91 or a variant thereof; (5e) a VH comprising CDR-H1 comprising the amino acid sequence of SEQ ID NO: 97 or a variant thereof; CDR-H2 comprising the amino acid sequence of SEQ ID NO: 107 or a variant thereof; and CDR-H3 comprising the amino acid sequence of SEQ ID NO: 110 or a variant thereof; and/or a VL comprising CDR-L1 comprising the amino acid sequence of SEQ ID NO: 116 or a variant thereof; CDR-L2 comprising the amino acid sequence of SEQ ID NO: 41 or a variant thereof; and CDR-L3 comprising the amino acid sequence of SEQ ID NO: 119 or a variant thereof; or (5f) a VH comprising CDR-H1 comprising the amino acid sequence of SEQ ID NO: 97 or a variant thereof; CDR-H2 comprising the amino acid sequence of SEQ ID NO: 102 or a variant thereof; and CDR-H3 comprising the amino acid sequence of SEQ ID NO: 110 or a variant thereof; and/or a VL comprising CDR-L1 comprising the amino acid sequence of SEQ ID NO: 113 or a variant thereof; CDR-L2 comprising the amino acid sequence of SEQ ID NO: 41 or a variant thereof; and CDR-L3 comprising the amino acid sequence of SEQ ID NO: 119 or a variant thereof, wherein the variant in any one of (1a), (1b), (1c), (1d), (1e), (1f), (1g), (1h), (2a), (2b), (2c), (2d), (2e), (2f), (2g), (2h), (3a), (3b), (3c), (3d), (3e), (3f), (3g), (3h), (4a), (4b), (4c), (4d), (4e), (4f), (4g), (4h), (5a), (5b), (5c), (5d), (5e) and (5f) has a substitution, deletion or addition of one, two or several amino acids (for example, a substitution, deletion or addition of 1, 2 or 3 amino acids) compared to the sequence from which it is derived; and in particular embodiments, the substitution is a conservative substitution.

In some embodiments, the antibody or the antigen-binding fragment thereof comprises: (1) the following VH and/or VL, wherein the CDRs are defined according to the Chothia numbering system: (1a) a VH comprising CDR-H1 comprising the amino acid sequence of SEQ ID NO: 17; CDR-H2 comprising the amino acid sequence of SEQ ID NO: 27; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42; (1b) a VH comprising CDR-H1 comprising the amino acid sequence of SEQ ID NO: 17; CDR-H2 comprising the amino acid sequence of SEQ ID NO: 22; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42; (1c) a VH comprising CDR-H1 comprising the amino acid sequence of SEQ ID NO: 44; CDR-H2 comprising the amino acid sequence of SEQ ID NO: 49; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 63; and CDR-L3 comprising the amino acid sequence of SEQ ID NO: 66; (1d) a VH comprising CDR-H1 comprising the amino acid sequence of SEQ ID NO: 44; CDR-H2 comprising the amino acid sequence of SEQ ID NO: 49; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 61; and CDR-L3 comprising the amino acid sequence of SEQ ID NO: 66; (1e) a VH comprising CDR-H1 comprising the amino acid sequence of SEQ ID NO: 68; CDR-H2 comprising the amino acid sequence of SEQ ID NO: 73; and CDR-H3 comprising the amino acid sequence of SEQ ID NO: 80; and/or a VL comprising CDR-L1 comprising the amino acid sequence of SEQ ID NO: 84; CDR-L2 comprising the amino acid sequence of SEQ ID NO: 88; and CDR-L3 comprising the amino acid sequence of SEQ ID NO: 91; (1f) a VH comprising CDR-H1 comprising the amino acid sequence of SEQ ID NO: 68; CDR-H2 comprising the amino acid sequence of SEQ ID NO: 73; and CDR-H3 comprising the amino acid sequence of SEQ ID NO: 80; and/or a VL comprising CDR-L1 comprising the amino acid sequence of SEQ ID NO: 83; CDR-L2 comprising the amino acid sequence of SEQ ID NO: 87; and CDR-L3 comprising the amino acid sequence of SEQ ID NO: 91; (1g) a VH comprising CDR-H1 comprising the amino acid sequence of SEQ ID NO: 93; CDR-H2 comprising the amino acid sequence of SEQ ID NO: 103; and CDR-H3 comprising the amino acid sequence of SEQ ID NO: 108; and/or a VL comprising CDR-L1 comprising the amino acid sequence of SEQ ID NO: 114; CDR-L2 comprising the amino acid sequence of SEQ ID NO: 117; and CDR-L3 comprising the amino acid sequence of SEQ ID NO: 119; or (1h) a VH comprising CDR-H1 comprising the amino acid sequence of SEQ ID NO: 93; CDR-H2 comprising the amino acid sequence of SEQ ID NO: 98; and CDR-H3 comprising the amino acid sequence of SEQ ID NO: 108; and/or a VL comprising CDR-L1 comprising the amino acid sequence of SEQ ID NO: 111; CDR-L2 comprising the amino acid sequence of SEQ ID NO: 117; and CDR-L3 comprising the amino acid sequence of SEQ ID NO: 119; or (2) the following VH and/or VL, wherein the CDRs are defined according to the AbM numbering system: (2a) a VH comprising CDR-H1 comprising the amino acid sequence of SEQ ID NO: 18; CDR-H2 comprising the amino acid sequence of SEQ ID NO: 28; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42; (2b) a VH comprising CDR-H1 comprising the amino acid sequence of SEQ ID NO: 18; CDR-H2 comprising the amino acid sequence of SEQ ID NO: 23; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42; (2c) a VH comprising CDR-H1 comprising the amino acid sequence of SEQ ID NO: 45; CDR-H2 comprising the amino acid sequence of SEQ ID NO: 50; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 63; and CDR-L3 comprising the amino acid sequence of SEQ ID NO: 66; (2d) a VH comprising CDR-H1 comprising the amino acid sequence of SEQ ID NO: 45; CDR-H2 comprising the amino acid sequence of SEQ ID NO: 50; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 61; and CDR-L3 comprising the amino acid sequence of SEQ ID NO: 66; (2e) a VH comprising CDR-H1 comprising the amino acid sequence of SEQ ID NO: 69; CDR-H2 comprising the amino acid sequence of SEQ ID NO: 74; and CDR-H3 comprising the amino acid sequence of SEQ ID NO: 80; and/or a VL comprising CDR-L1 comprising the amino acid sequence of SEQ ID NO: 84; CDR-L2 comprising the amino acid sequence of SEQ ID NO: 88; and CDR-L3 comprising the amino acid sequence of SEQ ID NO: 91; (2f) a VH comprising CDR-H1 comprising the amino acid sequence of SEQ ID NO: 69; CDR-H2 comprising the amino acid sequence of SEQ ID NO: 74; and CDR-H3 comprising the amino acid sequence of SEQ ID NO: 80; and/or a VL comprising CDR-L1 comprising the amino acid sequence of SEQ ID NO: 83; CDR-L2 comprising the amino acid sequence of SEQ ID NO: 87; and CDR-L3 comprising the amino acid sequence of SEQ ID NO: 91; (2g) a VH comprising CDR-H1 comprising the amino acid sequence of SEQ ID NO: 94; CDR-H2 comprising the amino acid sequence of SEQ ID NO: 104; and CDR-H3 comprising the amino acid sequence of SEQ ID NO: 108; and/or a VL comprising CDR-L1 comprising the amino acid sequence of SEQ ID NO: 114; CDR-L2 comprising the amino acid sequence of SEQ ID NO: 117; and CDR-L3 comprising the amino acid sequence of SEQ ID NO: 119; or (2h) a VH comprising CDR-H1 comprising the amino acid sequence of SEQ ID NO: 94; CDR-H2 comprising the amino acid sequence of SEQ ID NO: 99; and CDR-H3 comprising the amino acid sequence of SEQ ID NO: 108; and/or a VL comprising CDR-L1 comprising the amino acid sequence of SEQ ID NO: 111; CDR-L2 comprising the amino acid sequence of SEQ ID NO: 117; and CDR-L3 comprising the amino acid sequence of SEQ ID NO: 119; or (3) the following VH and/or VL, wherein the CDRs are defined according to the Kabat numbering system: (3a) a VH comprising CDR-H1 comprising the amino acid sequence of SEQ ID NO: 19; CDR-H2 comprising the amino acid sequence of SEQ ID NO: 29; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42; (3b) a VH comprising CDR-H1 comprising the amino acid sequence of SEQ ID NO: 19; CDR-H2 comprising the amino acid sequence of SEQ ID NO: 24; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42; (3c) a VH comprising CDR-H1 comprising the amino acid sequence of SEQ ID NO: 46; CDR-H2 comprising the amino acid sequence of SEQ ID NO: 52; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 63; and CDR-L3 comprising the amino acid sequence of SEQ ID NO: 66; (3d) a VH comprising CDR-H1 comprising the amino acid sequence of SEQ ID NO: 46; CDR-H2 comprising the amino acid sequence of SEQ ID NO: 51; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 61; and CDR-L3 comprising the amino acid sequence of SEQ ID NO: 66; (3e) a VH comprising CDR-H1 comprising the amino acid sequence of SEQ ID NO: 70; CDR-H2 comprising the amino acid sequence of SEQ ID NO: 77; and CDR-H3 comprising the amino acid sequence of SEQ ID NO: 80; and/or a VL comprising CDR-L1 comprising the amino acid sequence of SEQ ID NO: 84; CDR-L2 comprising the amino acid sequence of SEQ ID NO: 88; and CDR-L3 comprising the amino acid sequence of SEQ ID NO: 91; (3f) a VH comprising CDR-H1 comprising the amino acid sequence of SEQ ID NO: 70; CDR-H2 comprising the amino acid sequence of SEQ ID NO: 75; and CDR-H3 comprising the amino acid sequence of SEQ ID NO: 80; and/or a VL comprising CDR-L1 comprising the amino acid sequence of SEQ ID NO: 83; CDR-L2 comprising the amino acid sequence of SEQ ID NO: 87; and CDR-L3 comprising the amino acid sequence of SEQ ID NO: 91; (3g) a VH comprising CDR-H1 comprising the amino acid sequence of SEQ ID NO: 95; CDR-H2 comprising the amino acid sequence of SEQ ID NO: 105; and CDR-H3 comprising the amino acid sequence of SEQ ID NO: 108; and/or a VL comprising CDR-L1 comprising the amino acid sequence of SEQ ID NO: 114; CDR-L2 comprising the amino acid sequence of SEQ ID NO: 117; and CDR-L3 comprising the amino acid sequence of SEQ ID NO: 119; or (3h) a VH comprising CDR-H1 comprising the amino acid sequence of SEQ ID NO: 95; CDR-H2 comprising the amino acid sequence of SEQ ID NO: 100; and CDR-H3 comprising the amino acid sequence of SEQ ID NO: 108; and/or a VL comprising CDR-L1 comprising the amino acid sequence of SEQ ID NO: 111; CDR-L2 comprising the amino acid sequence of SEQ ID NO: 117; and CDR-L3 comprising the amino acid sequence of SEQ ID NO: 119; or (4) the following VH and/or VL, wherein the CDRs are defined according to the Contact numbering system: (4a) a VH comprising CDR-H1 comprising the amino acid sequence of SEQ ID NO: 20; CDR-H2 comprising the amino acid sequence of SEQ ID NO: 30; and CDR-H3 comprising the amino acid sequence of SEQ ID NO: 33; and/or a VL comprising CDR-L1 comprising the amino acid sequence of SEQ ID NO: 36; CDR-L2 comprising the amino acid sequence of SEQ ID NO: 40; and CDR-L3 comprising the amino acid sequence of SEQ ID NO: 43; (4b) a VH comprising CDR-H1 comprising the amino acid sequence of SEQ ID NO: 20; CDR-H2 comprising the amino acid sequence of SEQ ID NO: 25; and CDR-H3 comprising the amino acid sequence of SEQ ID NO: 33; and/or a VL comprising CDR-L1 comprising the amino acid sequence of SEQ ID NO: 36; CDR-L2 comprising the amino acid sequence of SEQ ID NO: 39; and CDR-L3 comprising the amino acid sequence of SEQ ID NO: 43; (4c) a VH comprising CDR-H1 comprising the amino acid sequence of SEQ ID NO: 47; CDR-H2 comprising the amino acid sequence of SEQ ID NO: 53; and CDR-H3 comprising the amino acid sequence of SEQ ID NO: 56; and/or a VL comprising CDR-L1 comprising the amino acid sequence of SEQ ID NO: 59; CDR-L2 comprising the amino acid sequence of SEQ ID NO: 64; and CDR-L3 comprising the amino acid sequence of SEQ ID NO: 67; (4d) a VH comprising CDR-H1 comprising the amino acid sequence of SEQ ID NO: 47; CDR-H2 comprising the amino acid sequence of SEQ ID NO: 53; and CDR-H3 comprising the amino acid sequence of SEQ ID NO: 56; and/or a VL comprising CDR-L1 comprising the amino acid sequence of SEQ ID NO: 59; CDR-L2 comprising the amino acid sequence of SEQ ID NO: 62; and CDR-L3 comprising the amino acid sequence of SEQ ID NO: 67; (4e) a VH comprising CDR-H1 comprising the amino acid sequence of SEQ ID NO: 71; CDR-H2 comprising the amino acid sequence of SEQ ID NO: 78; and CDR-H3 comprising the amino acid sequence of SEQ ID NO: 81; and/or a VL comprising CDR-L1 comprising the amino acid sequence of SEQ ID NO: 85; CDR-L2 comprising the amino acid sequence of SEQ ID NO: 89; and CDR-L3 comprising the amino acid sequence of SEQ ID NO: 92; (4f) a VH comprising CDR-H1 comprising the amino acid sequence of SEQ ID NO: 71; CDR-H2 comprising the amino acid sequence of SEQ ID NO: 76; and CDR-H3 comprising the amino acid sequence of SEQ ID NO: 81; and/or a VL comprising CDR-L1 comprising the amino acid sequence of SEQ ID NO: 85; CDR-L2 comprising the amino acid sequence of SEQ ID NO: 89; and CDR-L3 comprising the amino acid sequence of SEQ ID NO: 92; (4g) a VH comprising CDR-H1 comprising the amino acid sequence of SEQ ID NO: 96; CDR-H2 comprising the amino acid sequence of SEQ ID NO: 106; and CDR-H3 comprising the amino acid sequence of SEQ ID NO: 109; and/or a VL comprising CDR-L1 comprising the amino acid sequence of SEQ ID NO: 115; CDR-L2 comprising the amino acid sequence of SEQ ID NO: 118; and CDR-L3 comprising the amino acid sequence of SEQ ID NO: 120; or (4h) a VH comprising CDR-H1 comprising the amino acid sequence of SEQ ID NO: 96; CDR-H2 comprising the amino acid sequence of SEQ ID NO: 101; and CDR-H3 comprising the amino acid sequence of SEQ ID NO: 109; and/or a VL comprising CDR-L1 comprising the amino acid sequence of SEQ ID NO: 112; CDR-L2 comprising the amino acid sequence of SEQ ID NO: 118; and CDR-L3 comprising the amino acid sequence of SEQ ID NO: 120; or (5) the following VH and/or VL, wherein the CDRs are defined according to the IMGT numbering system: (5a) a VH comprising CDR-H1 comprising the amino acid sequence of SEQ ID NO: 21; CDR-H2 comprising the amino acid sequence of SEQ ID NO: 31; and CDR-H3 comprising the amino acid sequence of SEQ ID NO: 34; and/or a VL comprising CDR-L1 comprising the amino acid sequence of SEQ ID NO: 37; CDR-L2 comprising the amino acid sequence of SEQ ID NO: 41; and CDR-L3 comprising the amino acid sequence of SEQ ID NO: 42; (5b) a VH comprising CDR-H1 comprising the amino acid sequence of SEQ ID NO: 21; CDR-H2 comprising the amino acid sequence of SEQ ID NO: 26; and CDR-H3 comprising the amino acid sequence of SEQ ID NO: 34; and/or a VL comprising CDR-L1 comprising the amino acid sequence of SEQ ID NO: 37; CDR-L2 comprising the amino acid sequence of SEQ ID NO: 41; and CDR-L3 comprising the amino acid sequence of SEQ ID NO: 42; (5c) a VH comprising CDR-H1 comprising the amino acid sequence of SEQ ID NO: 48; CDR-H2 comprising the amino acid sequence of SEQ ID NO: 54; and CDR-H3 comprising the amino acid sequence of SEQ ID NO: 57; and/or a VL comprising CDR-L1 comprising the amino acid sequence of SEQ ID NO: 60; CDR-L2 comprising the amino acid sequence of SEQ ID NO: 65; and CDR-L3 comprising the amino acid sequence of SEQ ID NO: 66; (5d) a VH comprising CDR-H1 comprising the amino acid sequence of SEQ ID NO: 72; CDR-H2 comprising the amino acid sequence of SEQ ID NO: 79; and CDR-H3 comprising the amino acid sequence of SEQ ID NO: 82; and/or a VL comprising CDR-L1 comprising the amino acid sequence of SEQ ID NO: 86; CDR-L2 comprising the amino acid sequence of SEQ ID NO: 90; and CDR-L3 comprising the amino acid sequence of SEQ ID NO: 91; (5e) a VH comprising CDR-H1 comprising the amino acid sequence of SEQ ID NO: 97; CDR-H2 comprising the amino acid sequence of SEQ ID NO: 107; and CDR-H3 comprising the amino acid sequence of SEQ ID NO: 110; and/or a VL comprising CDR-L1 comprising the amino acid sequence of SEQ ID NO: 116; CDR-L2 comprising the amino acid sequence of SEQ ID NO: 41; and CDR-L3 comprising the amino acid sequence of SEQ ID NO: 119; or (5f) a VH comprising CDR-H1 comprising the amino acid sequence of SEQ ID NO: 97; CDR-H2 comprising the amino acid sequence of SEQ ID NO: 102; and CDR-H3 comprising the amino acid sequence of SEQ ID NO: 110; and/or a VL comprising CDR-L1 comprising the amino acid sequence of SEQ ID NO: 113; CDR-L2 comprising the amino acid sequence of SEQ ID NO: 41; and CDR-L3 comprising the amino acid sequence of SEQ ID NO: 119.

In some embodiments, the antibody or the antigen-binding fragment thereof comprises: (a) a VH comprising the amino acid sequence set forth in SEQ ID NO: 3 or a variant thereof and/or a VL comprising the amino acid sequence set forth in SEQ ID NO: 4 or a variant thereof; (b) a VH comprising the amino acid sequence set forth in SEQ ID NO: 1 or a variant thereof and/or a VL comprising the amino acid sequence set forth in SEQ ID NO: 2 or a variant thereof; (c) a VH comprising the amino acid sequence set forth in SEQ ID NO: 7 or a variant thereof and/or a VL comprising the amino acid sequence set forth in SEQ ID NO: 8 or a variant thereof; (d) a VH comprising the amino acid sequence set forth in SEQ ID NO: 5 or a variant thereof and/or a VL comprising the amino acid sequence set forth in SEQ ID NO: 6 or a variant thereof; (e) a VH comprising the amino acid sequence set forth in SEQ ID NO: 11 or a variant thereof and/or a VL comprising the amino acid sequence set forth in SEQ ID NO: 12 or a variant thereof; (f) a VH comprising the amino acid sequence set forth in SEQ ID NO: 9 or a variant thereof and/or a VL comprising the amino acid sequence set forth in SEQ ID NO: 10 or a variant thereof; (g) a VH comprising the amino acid sequence set forth in SEQ ID NO: 15 or a variant thereof and/or a VL comprising the amino acid sequence set forth in SEQ ID NO: 16 or a variant thereof; or (h) a VH comprising the amino acid sequence set forth in SEQ ID NO: 13 or a variant thereof and/or a VL comprising the amino acid sequence set forth in SEQ ID NO: 14 or a variant thereof, wherein the variant has at least 80%, at least 85%, at least 90%, at least 91%, at least 92%, at least 93%, at least 94%, at least 95%, at least 96%, at least 97%, at least 98%, at least 99% or 100% sequence identity compared to the sequence from which it is derived, or has a substitution, deletion or addition of one, two or several amino acids (for example, a substitution, deletion or addition of 1, 2, 3, 4, or 5 amino acids) compared to the sequence from which it is derived; and in particular embodiments, the substitution is a conservative substitution.

In some embodiments, the antibody or the antigen-binding fragment thereof comprises (a) a VH comprising the amino acid sequence set forth in SEQ ID NO: 3 and a VL comprising the amino acid sequence set forth in SEQ ID NO: 4; (b) a VH comprising the amino acid sequence set forth in SEQ ID NO: 1 and a VL comprising the amino acid sequence set forth in SEQ ID NO: 2; (c) a VH comprising the amino acid sequence set forth in SEQ ID NO: 7 and a VL comprising the amino acid sequence set forth in SEQ ID NO: 8; (d) a VH comprising the amino acid sequence set forth in SEQ ID NO: 5 and a VL comprising the amino acid sequence set forth in SEQ ID NO: 6; (e) a VH comprising the amino acid sequence set forth in SEQ ID NO: 11 and a VL comprising the amino acid sequence set forth in SEQ ID NO: 12; (f) a VH comprising the amino acid sequence set forth in SEQ ID NO: 9 and a VL comprising the amino acid sequence set forth in SEQ ID NO: 10; (g) a VH comprising the amino acid sequence set forth in SEQ ID NO: 15 and a VL comprising the amino acid sequence set forth in SEQ ID NO: 16; or (h) a VH comprising the amino acid sequence set forth in SEQ ID NO: 13 and a VL comprising the amino acid sequence set forth in SEQ ID NO: 14.

In some embodiments, the antibody or the antigen-binding fragment thereof is a murine antibody, a chimeric antibody, or a humanized antibody.

In some embodiments, the antibody or the antigen-binding fragment thereof further comprises a constant region from or derived from human immunoglobulin. In some embodiments, the heavy chain of the antibody or the antigen-binding fragment thereof comprises a heavy chain constant region from or derived from the human immunoglobulin (for example, IgG1, IgG2, IgG3, or IgG4). In some embodiments, the antibody or the antigen-binding fragment thereof comprises a wild-type Fc region, or a mutated or chemically modified Fc region having an altered effector function compared to the wild-type Fc region. In some embodiments, the antibody or the antigen-binding fragment thereof comprises a variant of a human IgG1 heavy chain constant region, and the variant has the following substitutions compared to the wild-type sequence from which it is derived: Leu234Ala, Leu235Ala, and Gly237Ala (positions according to the EU numbering system).

In some embodiments, the light chain of the antibody or the antigen-binding fragment thereof comprises a light chain constant region (CL) from or derived from the human immunoglobulin (for example, x or X). In some embodiments, the antibody or the antigen-binding fragment thereof comprises a heavy chain constant region (CH) comprising the amino acid sequence set forth in SEQ ID NO: 121 or a variant thereof. In some embodiments, the variant has a conservative substitution of at most 20 amino acids (for example, a conservative substitution of at most 15, at most 10, or at most 5 amino acids; for example, a conservative substitution of 1, 2, 3, 4, or 5 amino acids) compared to SEQ ID NO: 121. In some embodiments, the antibody or the antigen-binding fragment thereof comprises a CL comprising the amino acid sequence set forth in SEQ ID NO: 123 or 144 or a variant thereof. In some embodiments, the variant has a conservative substitution of at most 20 amino acids (for example, a conservative substitution of at most 15, at most 10, or at most 5 amino acids; for example, a conservative substitution of 1, 2, 3, 4, or 5 amino acids) compared to SEQ ID NO: 123 or 144.

In some embodiments, the antibody or the antigen-binding fragment thereof comprises the variant of the human IgG1 heavy chain constant region comprising the amino acid sequence set forth in SEQ ID NO: 122. In some embodiments, the antibody or the antigen-binding fragment thereof comprises a CH comprising the amino acid sequence set forth in SEQ ID NO: 121 or 122 and a CL comprising the amino acid sequence set forth in SEQ ID NO: 123 or 144.

In some embodiments, the CH comprising the amino acid sequence set forth in SEQ ID NO: 121 or 122 or the variant thereof lacks a C-terminal lysine.

In some embodiments, the antibody or the antigen-binding fragment thereof comprises (1) a heavy chain comprising a VH comprising the amino acid sequence set forth in SEQ ID NO: 3 and a CH comprising the amino acid sequence set forth in SEQ ID NO: 122, and a light chain comprising a VL comprising the amino acid sequence set forth in SEQ ID NO: 4 and a CL comprising the amino acid sequence set forth in SEQ ID NO: 123; (2) a heavy chain comprising a VH comprising the amino acid sequence set forth in SEQ ID NO: 3 and a CH comprising the amino acid sequence set forth in SEQ ID NO: 121, and a light chain comprising a VL comprising the amino acid sequence set forth in SEQ ID NO: 4 and a CL comprising the amino acid sequence set forth in SEQ ID NO: 123; (3) a heavy chain comprising a VH comprising the amino acid sequence set forth in SEQ ID NO: 1 and a CH comprising the amino acid sequence set forth in SEQ ID NO: 121, and a light chain comprising a VL comprising the amino acid sequence set forth in SEQ ID NO: 2 and a CL comprising the amino acid sequence set forth in SEQ ID NO: 123; (4) a heavy chain comprising a VH comprising the amino acid sequence set forth in SEQ ID NO: 7 and a CH comprising the amino acid sequence set forth in SEQ ID NO: 121, and a light chain comprising a VL comprising the amino acid sequence set forth in SEQ ID NO: 8 and a CL comprising the amino acid sequence set forth in SEQ ID NO: 144; (5) a heavy chain comprising a VH comprising the amino acid sequence set forth in SEQ ID NO: 5 and a CH comprising the amino acid sequence set forth in SEQ ID NO: 121, and a light chain comprising a VL comprising the amino acid sequence set forth in SEQ ID NO: 6 and a CL comprising the amino acid sequence set forth in SEQ ID NO: 144; (6) a heavy chain comprising a VH comprising the amino acid sequence set forth in SEQ ID NO: 11 and a CH comprising the amino acid sequence set forth in SEQ ID NO: 121, and a light chain comprising a VL comprising the amino acid sequence set forth in SEQ ID NO: 12 and a CL comprising the amino acid sequence set forth in SEQ ID NO: 123; (7) a heavy chain comprising a VH comprising the amino acid sequence set forth in SEQ ID NO: 9 and a CH comprising the amino acid sequence set forth in SEQ ID NO: 121, and a light chain comprising a VL comprising the amino acid sequence set forth in SEQ ID NO: 10 and a CL comprising the amino acid sequence set forth in SEQ ID NO: 123; (8) a heavy chain comprising a VH comprising the amino acid sequence set forth in SEQ ID NO: 15 and a CH comprising the amino acid sequence set forth in SEQ ID NO: 121, and a light chain comprising a VL comprising the amino acid sequence set forth in SEQ ID NO: 16 and a CL comprising the amino acid sequence set forth in SEQ ID NO: 123; or (9) a heavy chain comprising a VH comprising the amino acid sequence set forth in SEQ ID NO: 13 and a CH comprising the amino acid sequence set forth in SEQ ID NO: 121, and a light chain comprising a VL comprising the amino acid sequence set forth in SEQ ID NO: 14 and a CL comprising the amino acid sequence set forth in SEQ ID NO: 123.

In some embodiments, an N-terminal glutamine of the VH comprising a sequence set forth in any one of SEQ ID NOs: 3, 1, 7, 5, 11, 9, 15 and 13 or a variant thereof is subjected to cyclization to form pyroglutamic acid or pyroglutamate.

In some embodiments, the antibody or the antigen-binding fragment thereof comprises (1) a heavy chain comprising the amino acid sequence set forth in SEQ ID NO: 126, and a light chain comprising the amino acid sequence set forth in SEQ ID NO: 125; (2) a heavy chain comprising the amino acid sequence set forth in SEQ ID NO: 124, and a light chain comprising the amino acid sequence set forth in SEQ ID NO: 125; (3) a heavy chain comprising the amino acid sequence set forth in SEQ ID NO: 127, and a light chain comprising the amino acid sequence set forth in SEQ ID NO: 128; (4) a heavy chain comprising the amino acid sequence set forth in SEQ ID NO: 129, and a light chain comprising the amino acid sequence set forth in SEQ ID NO: 130; (5) a heavy chain comprising the amino acid sequence set forth in SEQ ID NO: 131, and a light chain comprising the amino acid sequence set forth in SEQ ID NO: 132; (6) a heavy chain comprising the amino acid sequence set forth in SEQ ID NO: 133, and a light chain comprising the amino acid sequence set forth in SEQ ID NO: 134; (7) a heavy chain comprising the amino acid sequence set forth in SEQ ID NO: 135, and a light chain comprising the amino acid sequence set forth in SEQ ID NO: 136; (8) a heavy chain comprising the amino acid sequence set forth in SEQ ID NO: 137, and a light chain comprising the amino acid sequence set forth in SEQ ID NO: 138; or (9) a heavy chain comprising the amino acid sequence set forth in SEQ ID NO: 139, and a light chain comprising the amino acid sequence set forth in SEQ ID NO: 140.

In come embodiments, the heavy chain comprising the amino acid sequence set forth in any one of SEQ ID NOs: 126, 124, 127, 129, 131, 133, 135, 137, 139 has an N-terminal glutamine subjected to cyclization to form pyroglutamic acid or pyroglutamate; and/or the heavy chain comprising the amino acid sequence set forth in any one of SEQ ID NOs: 126, 124, 127, 129, 131, 133, 135, 137, 139 lacks a C-terminal lysine.

In some embodiments, the antibody comprises a heavy chain comprising the amino acid sequence set forth in SEQ ID NO: 141 and a light chain comprising the amino acid sequence set forth in SEQ ID NO: 125; a heavy chain comprising the amino acid sequence set forth in SEQ ID NO: 142 and a light chain comprising the amino acid sequence set forth in SEQ ID NO: 125; or a heavy chain comprising the amino acid sequence set forth in SEQ ID NO: 143 and a light chain comprising the amino acid sequence set forth in SEQ ID NO: 125.

In some embodiments, M is

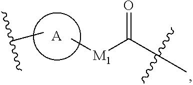

and wherein ring A is a 5-6-membered aliphatic heterocyclic ring or a 5-20-membered aromatic ring system, and the aliphatic heterocyclic ring and the aromatic ring system are optionally substituted with one or more groups selected from oxo (═O), halogen, cyano, amino, carboxyl, sulfhydryl, and C₁₋₆alkyl; M₁ is selected from a single bond, C₁₋₂₀alkylene, C₂₋₂₀alkenylene, and C₂₋₂₀alkynylene;
in particular embodiments, M is

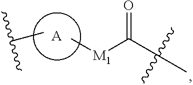

wherein ring A is a 5-membered aliphatic heterocyclic ring, a 6-membered heteroaromatic ring, or a polycyclic ring formed by connecting more than one 6-membered heteroaromatic ring with a phenyl ring via a single bond, and the aliphatic heterocyclic ring is optionally substituted with one or more groups selected from oxo (=O), halogen, and C₁₋₄alkyl; and M₁ is selected from a single bond, C₃₋₁₀alkylene, C₃₋₁₀alkenylene, and C₃₋₁₀alkynylene;
in particular embodiments, M is

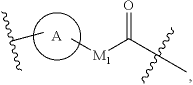

wherein ring A is selected from

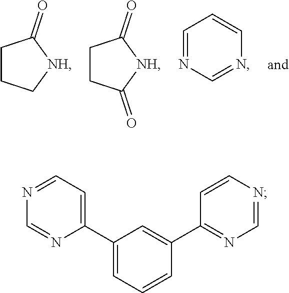

and M₁ is selected from a single bond, C₅₋₈alkylene, C₅₋₈alkenylene, and C₅₋₈alkynylene;
in particular embodiments, M is selected from the following structures:

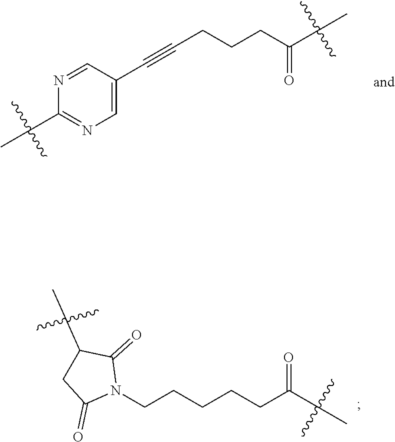 [SMILES: *C(=O)CCCC#Cc1cnc(*)nc1 ; *C(=O)CCCCCN1C(=O)CC(*)C1=O]

and
in particular embodiments, M has the following structure:

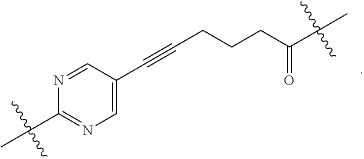 [SMILES: *C(=O)CCCC#Cc1cnc(*)nc1]

In some embodiments, L is selected from the structures comprising one or more of the following: C₁₋₆alkylene, —N(R′)—, carbonyl, —O—, natural amino acids or unnatural amino acids and analogs thereof (such as Ala, Arg, Asn, Asp, Cit, Cys, Gln, Glu, Gly, His, Ile, Leu, Lys, Met, Phe, Pro, Ser, Thr, Trp, Tyr, Val, Lys (COCH₂CH₂(OCH₂CH₂)ᵣOCH₃)), and short peptides consisting of amino acids (such as Ala-Ala, Ala-Lys, Ala-Lys(Ac), Ala-Pro, Gly-Glu, Gly-Gly, Phe-Lys, Phe-Lys(Ac), Val-Ala, Val-Lys, Val-Lys(Ac), Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly),

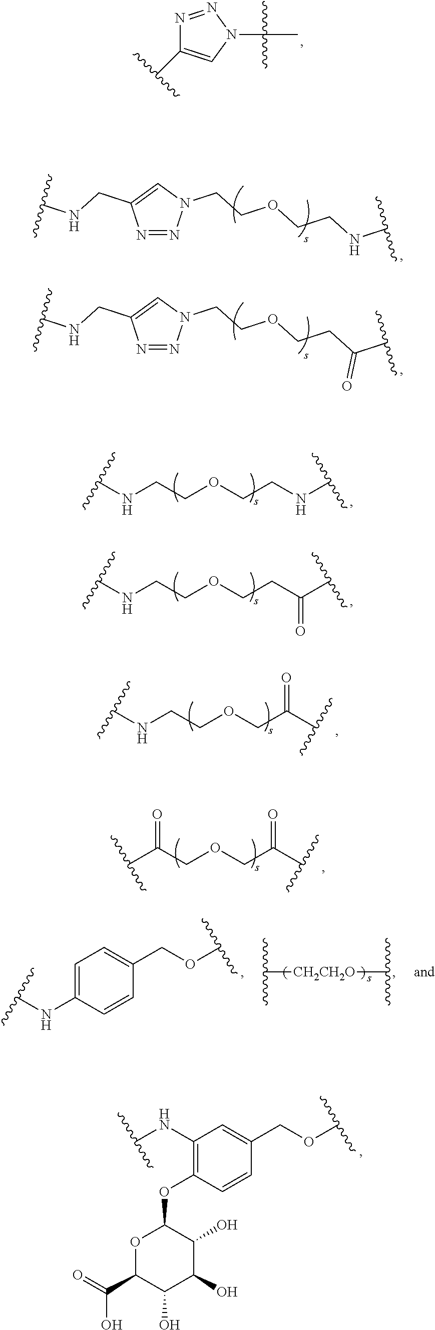

or a combination thereof; wherein R′ represents hydrogen, C₁₋₆alkyl, or a polyethylene glycol fragment containing 1-10 EO units; s is selected from integers of 1-20, for example, integers of 1-15, 1-10, 1-8, 1-6, 1-4, 1-2, and 3-6, for example, s is 1, 2, 3, 4, 5, 6, 7, 8, 9, 10, 11, 12, 13, 14, or 15;

in particular embodiments, L is selected from the structures comprising one or more of the following: C₁₋₆alkylene, carbonyl, —NH—, Ala-Ala, Ala-Lys, Ala-Pro, Gly-Glu, Gly-Gly, Phe-Lys, Val-Ala, Val-Lys, Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly,

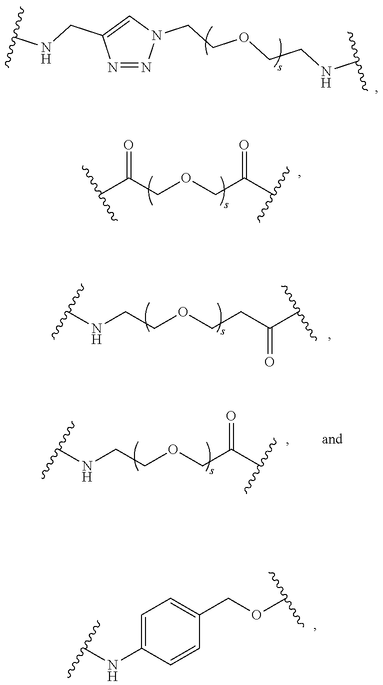

or a combination thereof; wherein s is selected from integers of 1-20, for example, integers of 1-15, 1-10, 1-8, 1-6, 1-4, 1-2, and 3-6, for example, s is 1, 2, 3, 4, 5, 6, 7, 8, 9, 10, 11, 12, 13, 14, or 15;
in particular embodiments, L is selected from the following structures:

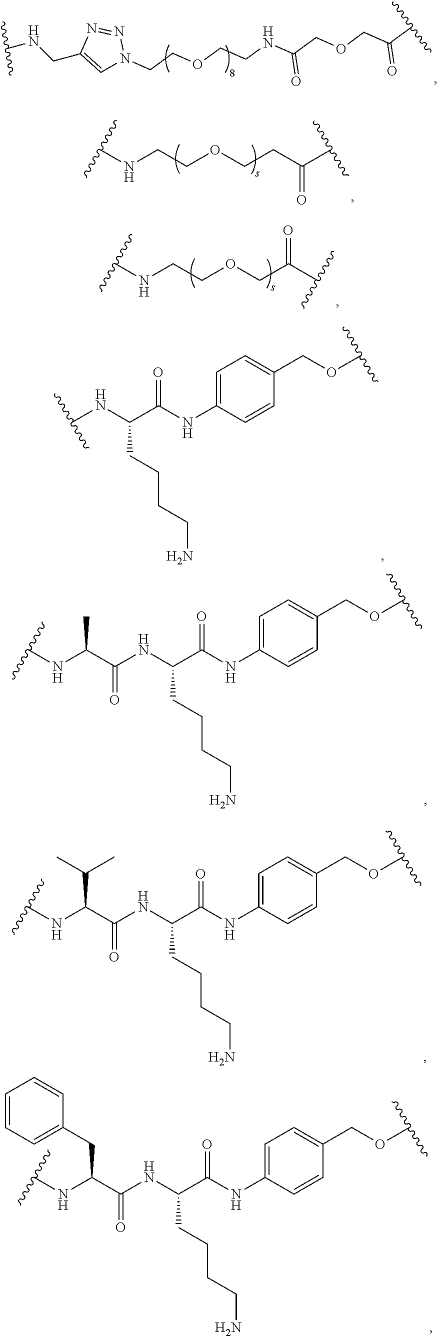

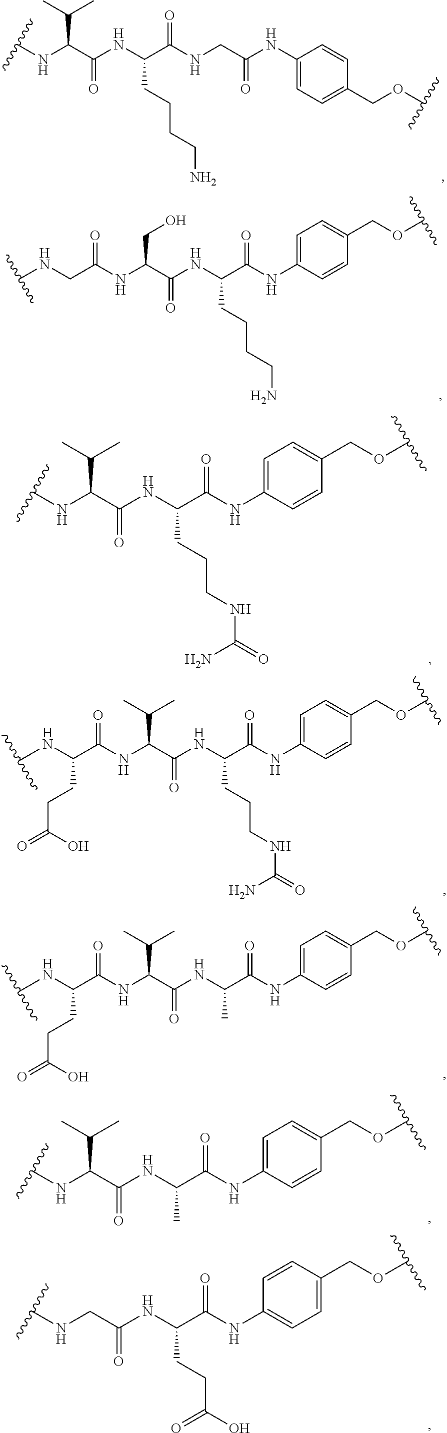

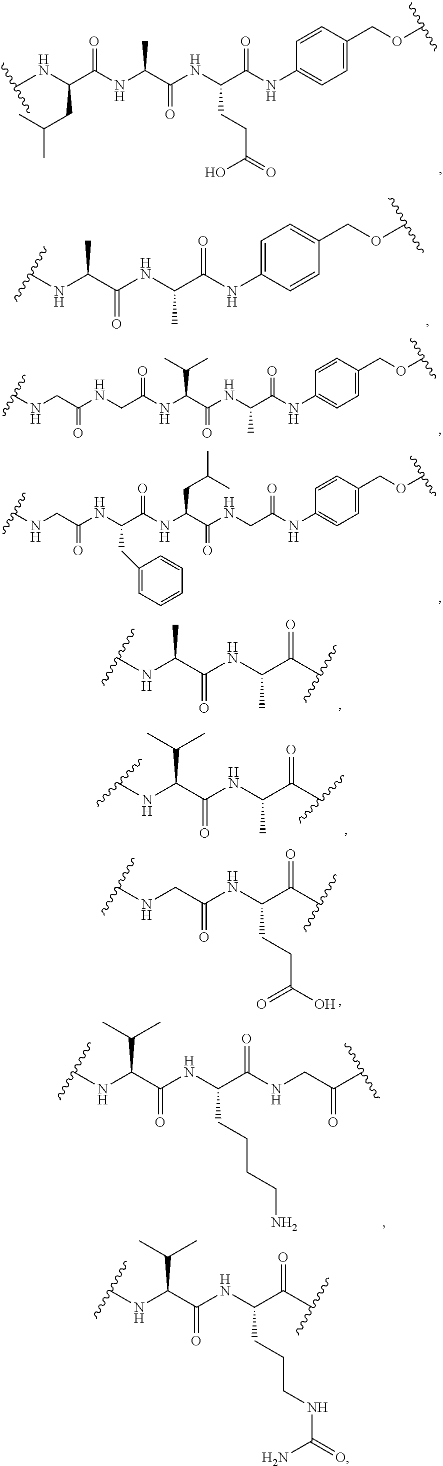

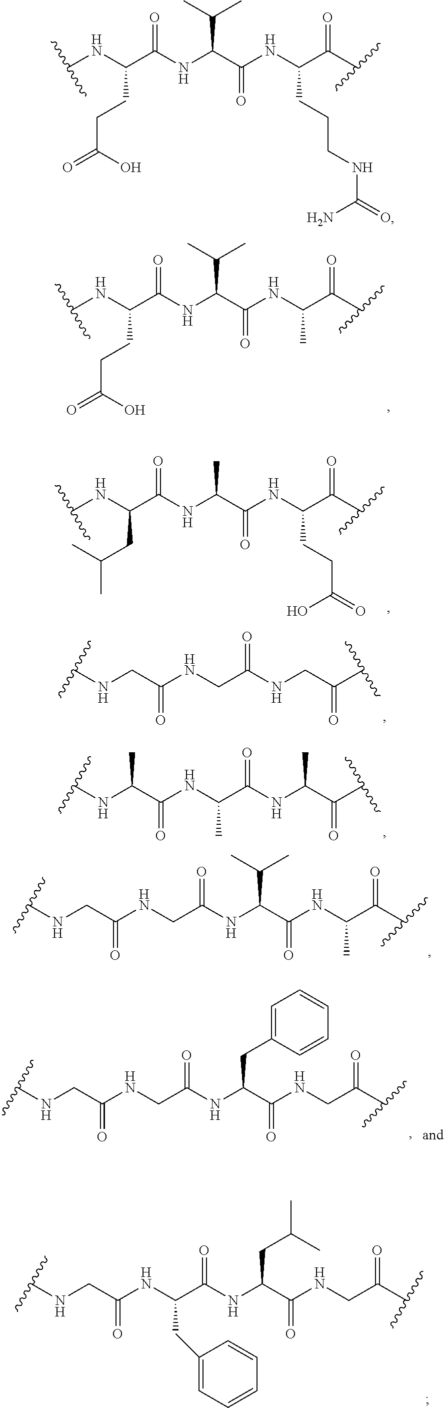

in particular embodiments, L is selected from the following structures:

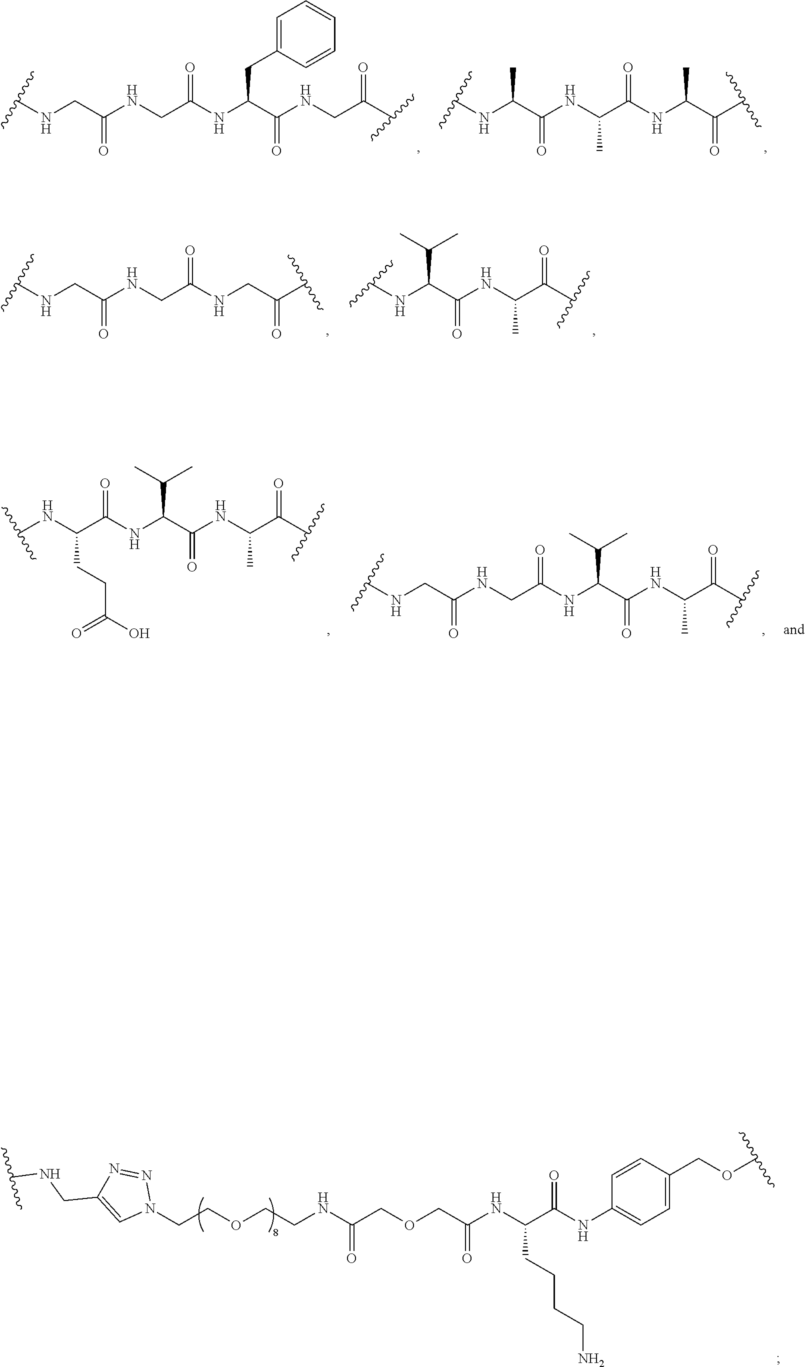

in particular embodiments, L is selected from the following structures:

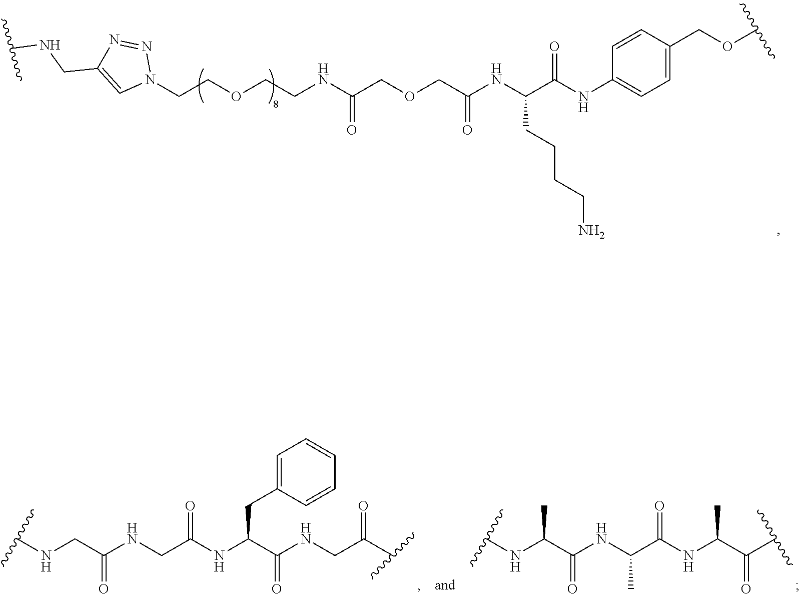

and
in particular embodiments, L is selected from the following structures:

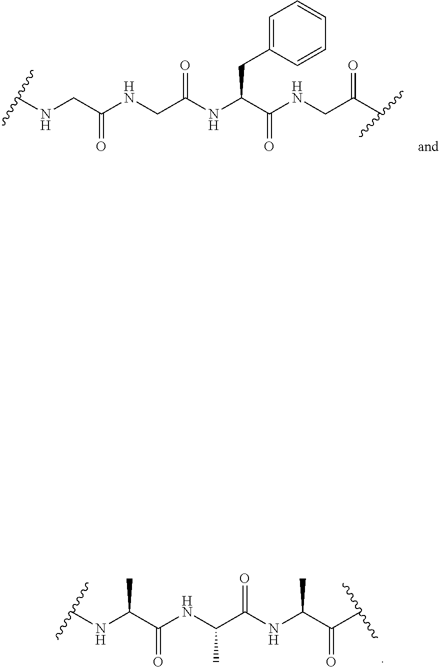 [SMILES: *NCC(=O)NCC(=O)N[C@@H](Cc1ccccc1)C(=O)NCC(*)=O ; *N[C@@H](C)C(=O)N[C@@H](C)C(=O)N[C@@H](C)C(*)=O]

In some embodiments, E is a single bond, —NH—CH₂—, —NH—CH₂—O—CH₂—CO—,

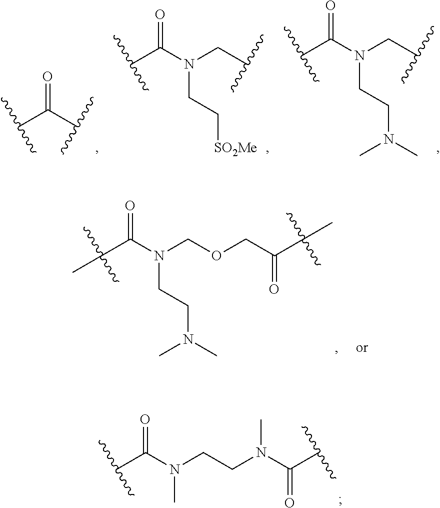

in particular embodiments, E is a single bond, —NH—CH₂—, —NH—CH₂—O—CH₂—CO—,

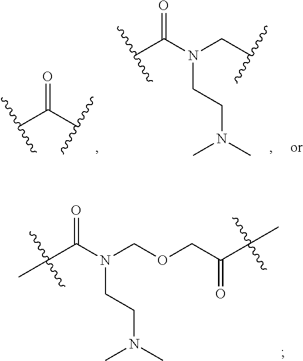

in particular embodiments, E is —NH—CH₂—,

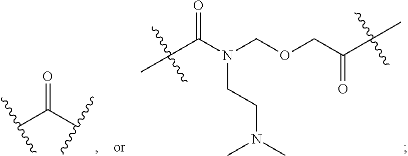

and
in particular embodiments, E is —NH—CH₂— or

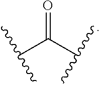 [SMILES: *C(*)=O]

In some embodiments,

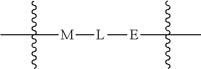

is selected from the following structures:

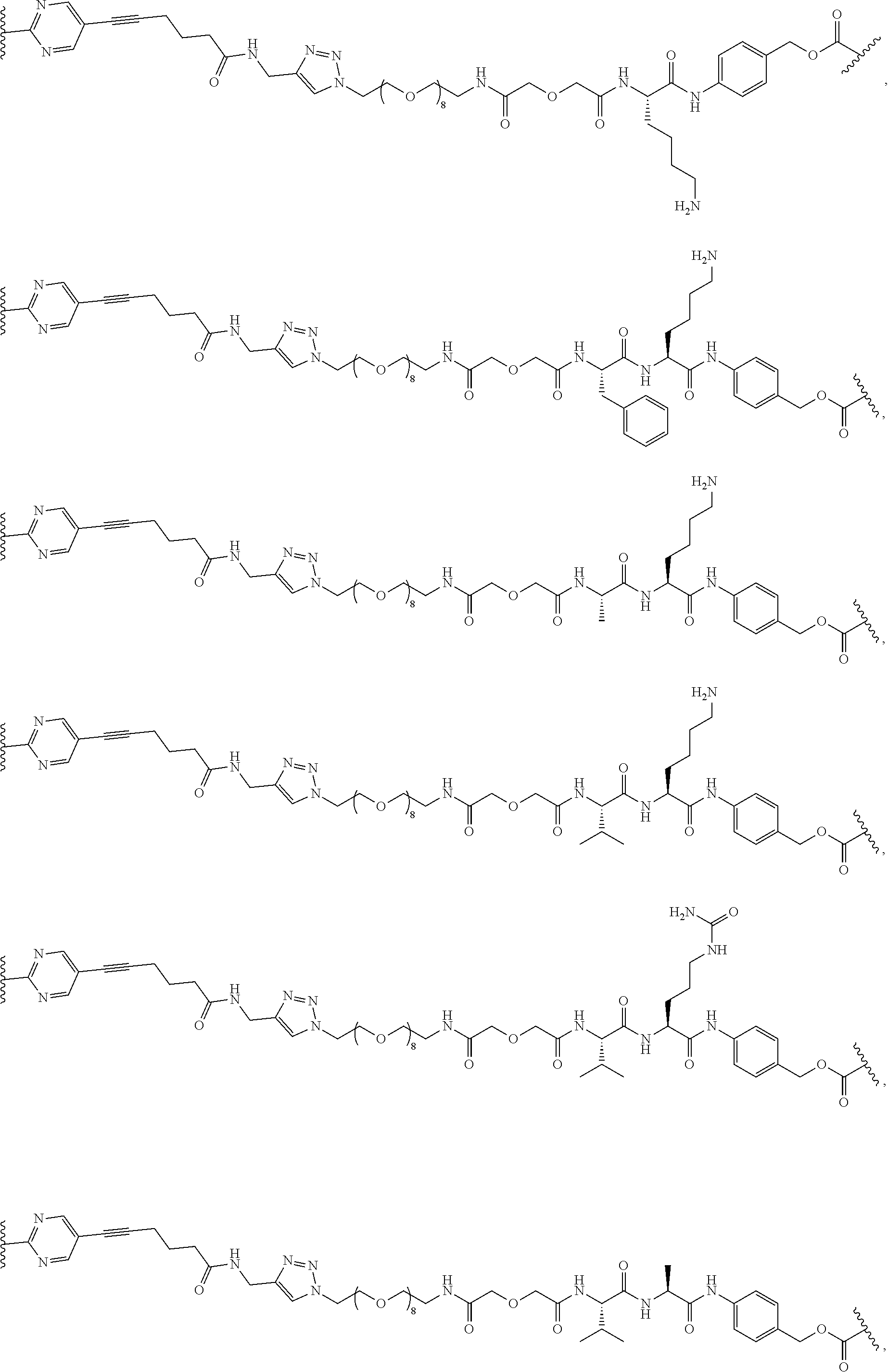

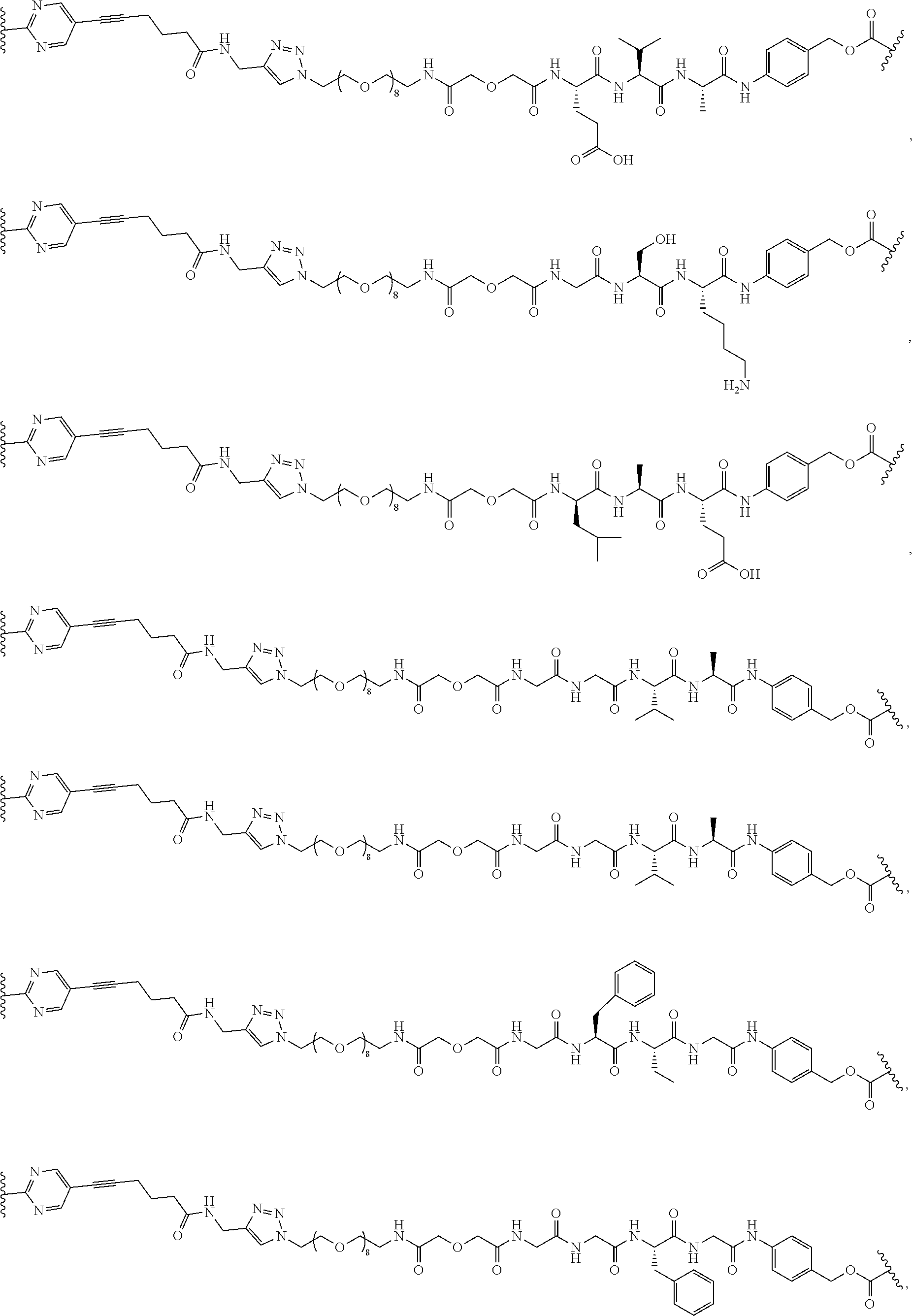

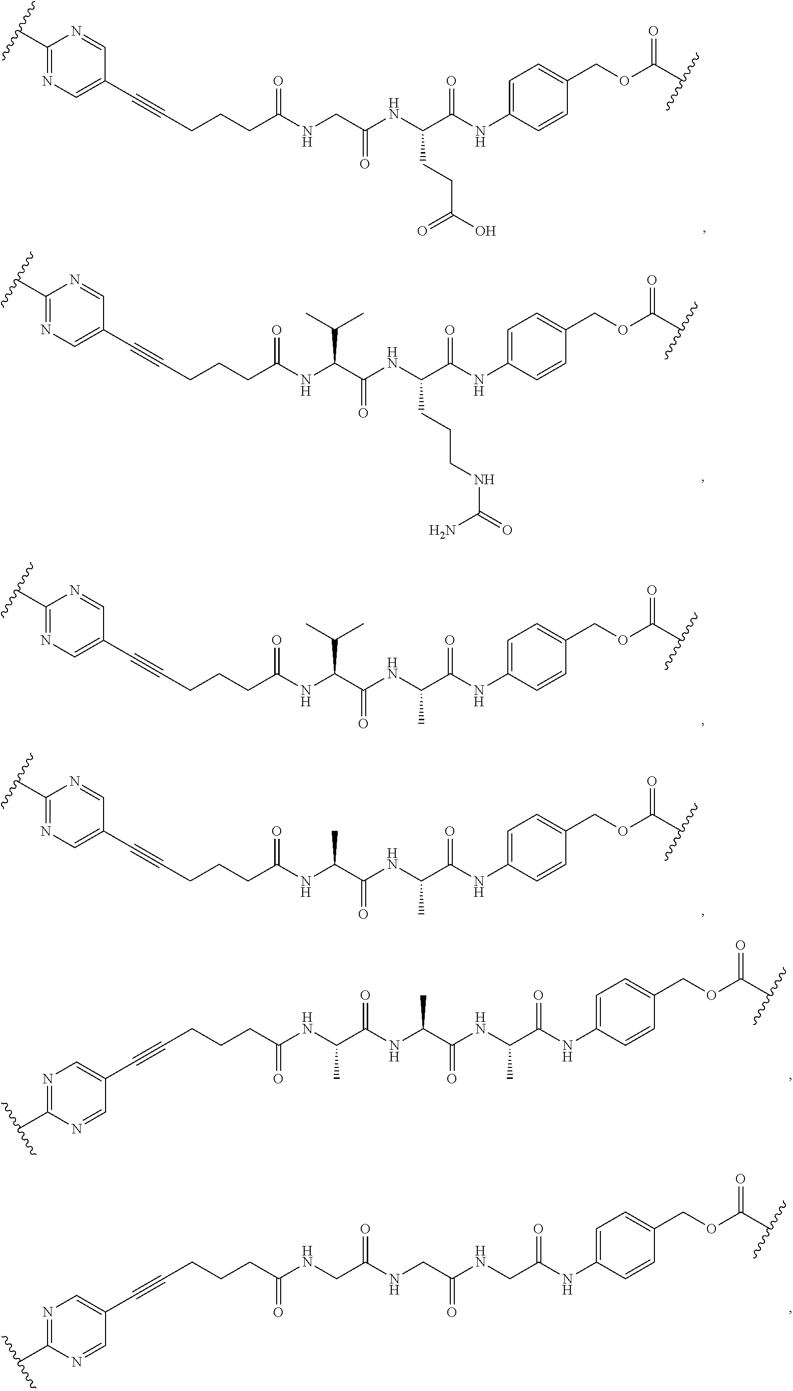 [SMILES: *C(=O)OCc1ccc(NC(=O)[C@H](CCC(=O)O)NC(=O)CNC(=O)CCCC#Cc2cnc(*)nc2)cc1 ; *C(=O)OCc1ccc(NC(=O)[C@H](CCCNC(N)=O)NC(=O)[C@@H](NC(=O)CCCC#Cc2cnc(*)nc2)C(C)C)cc1 ; *C(=O)OCc1ccc(NC(=O)[C@H](C)NC(=O)[C@@H](NC(=O)CCCC#Cc2cnc(*)nc2)C(C)C)cc1 ; *C(=O)OCc1ccc(NC(=O)[C@H](C)NC(=O)[C@H](C)NC(=O)CCCC#Cc2cnc(*)nc2)cc1 ; *C(=O)OCc1ccc(NC(=O)[C@H](C)NC(=O)[C@H](C)NC(=O)[C@H](C)NC(=O)CCCC#Cc2cnc(*)nc2)cc1 ; *C(=O)OCc1ccc(NC(=O)CNC(=O)CNC(=O)CNC(=O)CCCC#Cc2cnc(*)nc2)cc1]

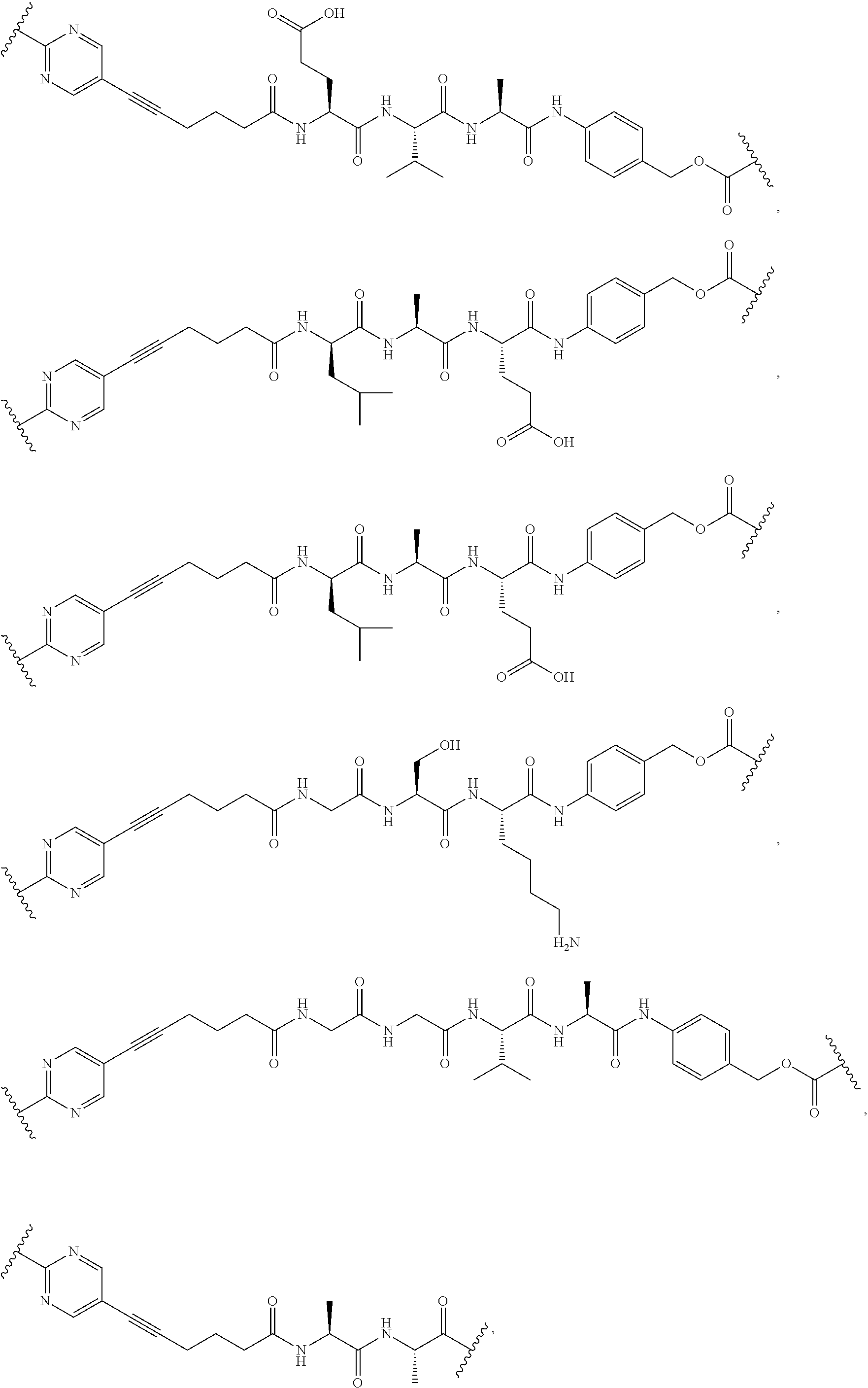

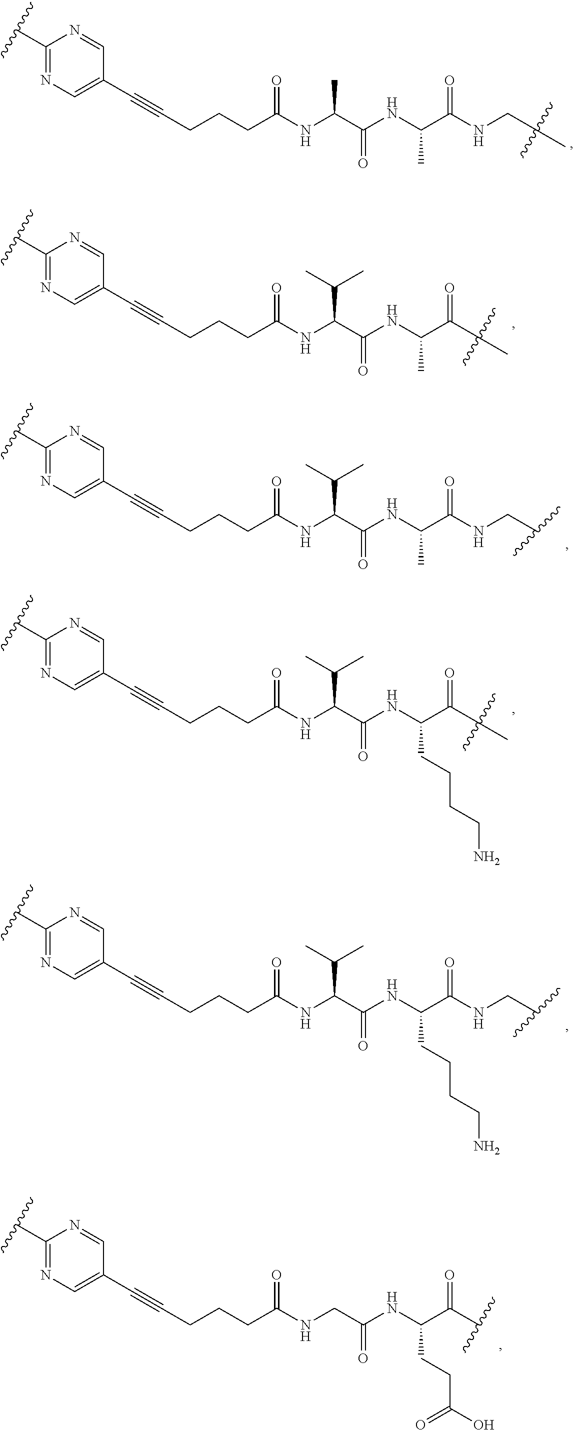 [SMILES: *CNC(=O)[C@H](C)NC(=O)[C@H](C)NC(=O)CCCC#Cc1cnc(*)nc1 ; *C(=O)[C@H](C)NC(=O)[C@@H](NC(=O)CCCC#Cc1cnc(*)nc1)C(C)C ; *CNC(=O)[C@H](C)NC(=O)[C@@H](NC(=O)CCCC#Cc1cnc(*)nc1)C(C)C ; *C(=O)[C@H](CCCCN)NC(=O)[C@@H](NC(=O)CCCC#Cc1cnc(*)nc1)C(C)C ; *CNC(=O)[C@H](CCCCN)NC(=O)[C@@H](NC(=O)CCCC#Cc1cnc(*)nc1)C(C)C ; *C(=O)[C@H](CCC(=O)O)NC(=O)CNC(=O)CCCC#Cc1cnc(*)nc1]

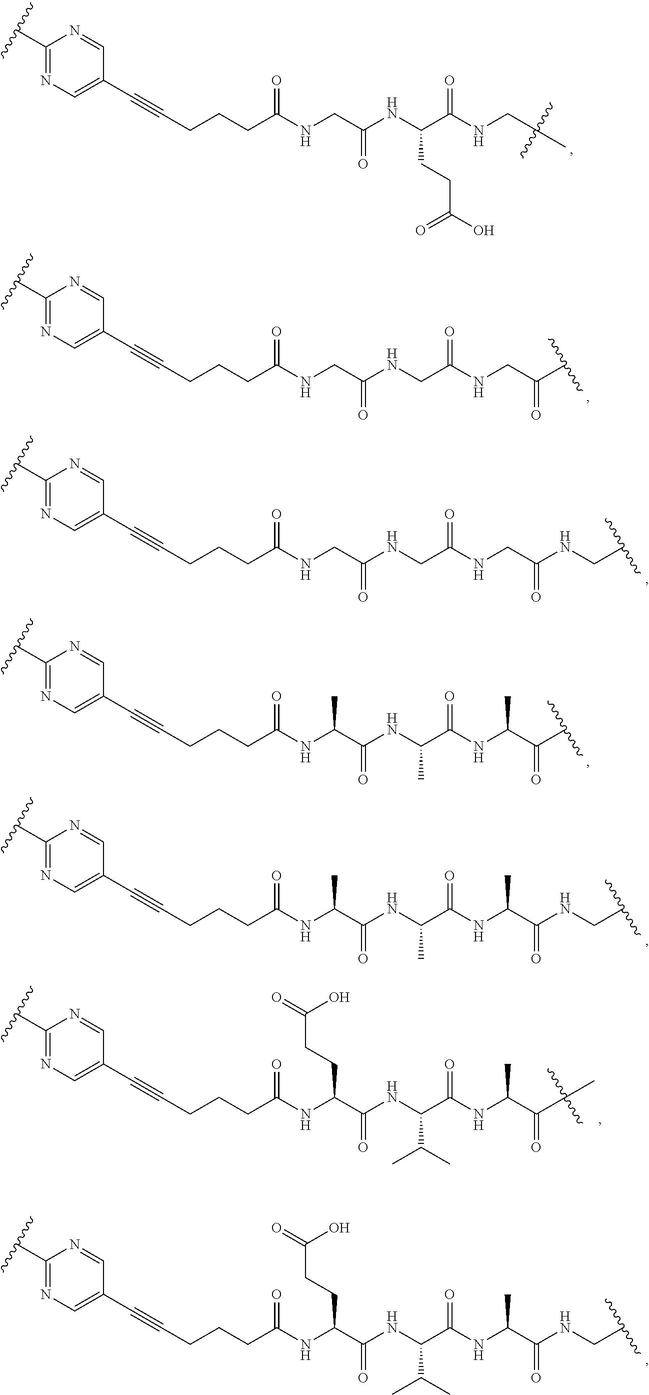

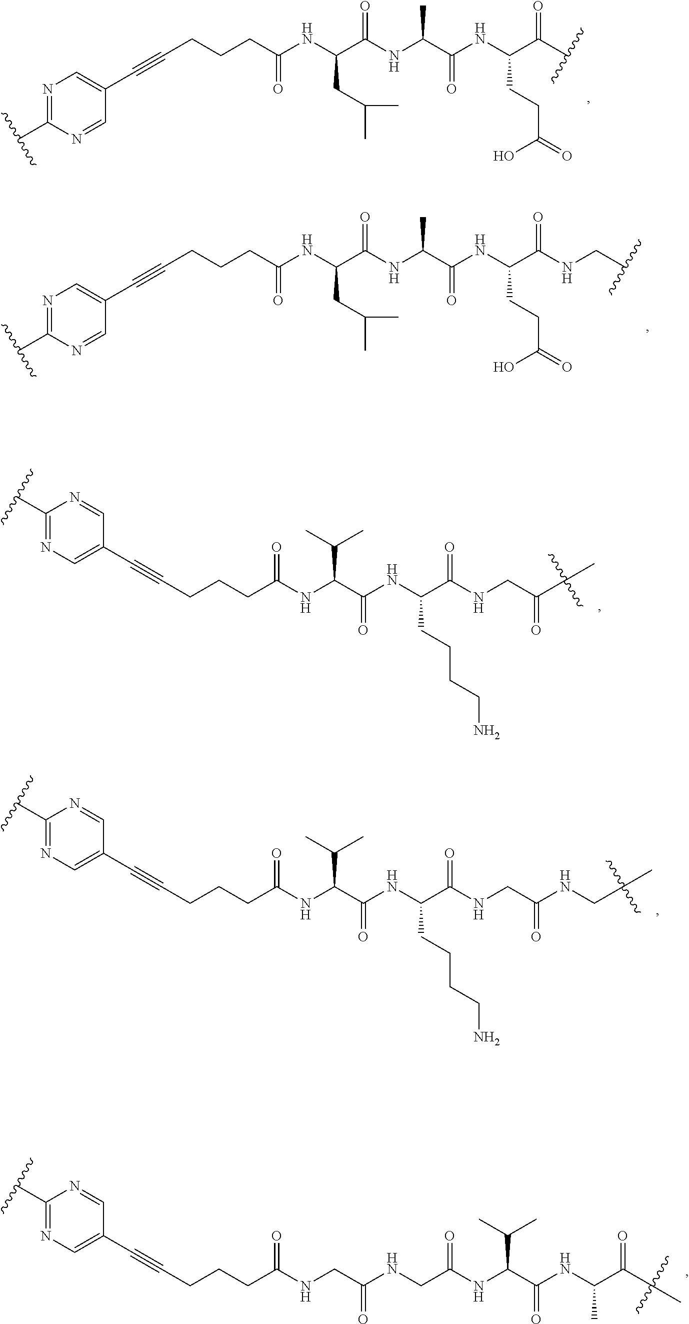 [SMILES: *C(=O)[C@H](CCC(=O)O)NC(=O)[C@H](C)NC(=O)[C@@H](CC(C)C)NC(=O)CCCC#Cc1cnc(*)nc1 ; *CNC(=O)[C@H](CCC(=O)O)NC(=O)[C@H](C)NC(=O)[C@@H](CC(C)C)NC(=O)CCCC#Cc1cnc(*)nc1 ; *C(=O)CNC(=O)[C@H](CCCCN)NC(=O)[C@@H](NC(=O)CCCC#Cc1cnc(*)nc1)C(C)C ; *CNC(=O)CNC(=O)[C@H](CCCCN)NC(=O)[C@@H](NC(=O)CCCC#Cc1cnc(*)nc1)C(C)C ; *C(=O)[C@H](C)NC(=O)[C@@H](NC(=O)CNC(=O)CNC(=O)CCCC#Cc1cnc(*)nc1)C(C)C]

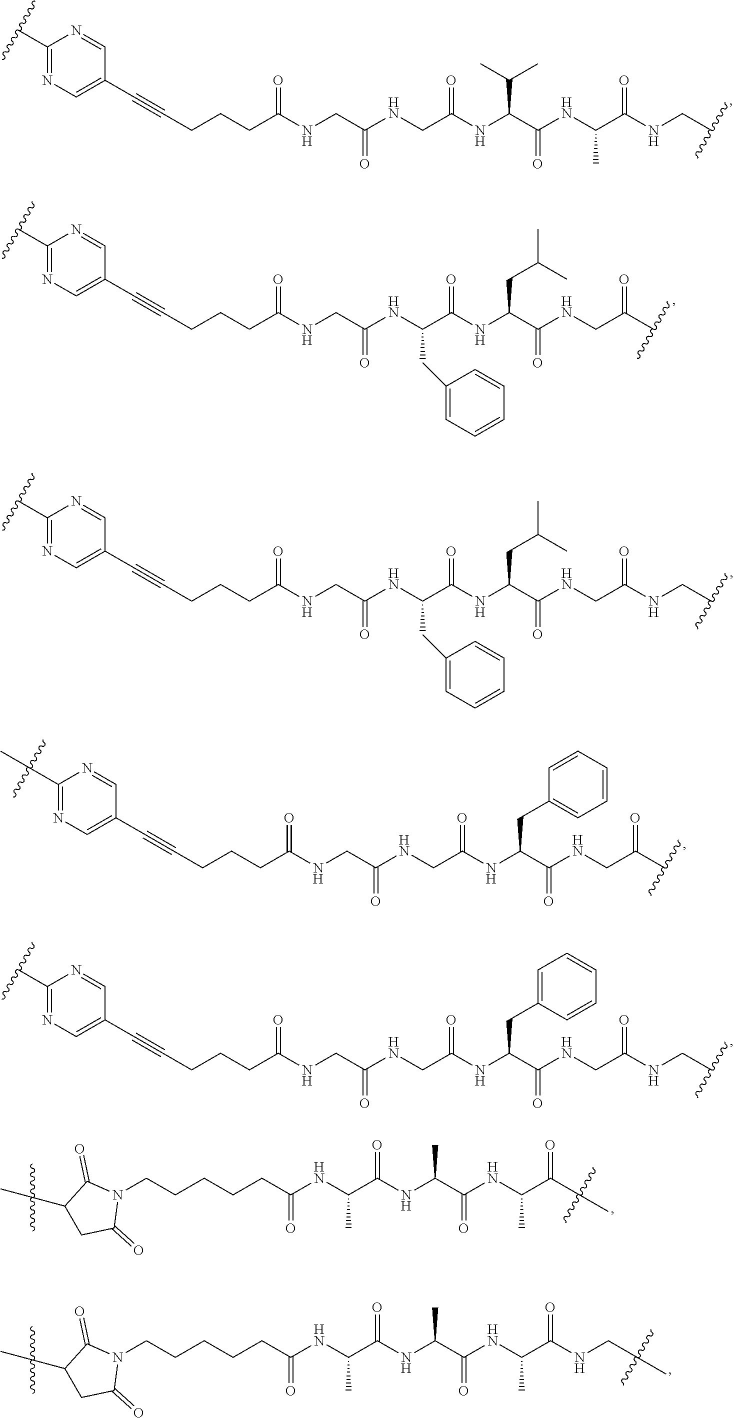

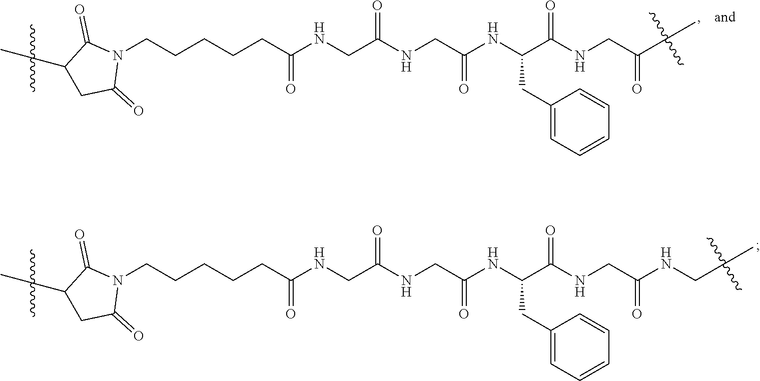 [SMILES: *C(=O)CNC(=O)[C@H](Cc1ccccc1)NC(=O)CNC(=O)CNC(=O)CCCCCN1C(=O)CC(*)C1=O ; *CNC(=O)CNC(=O)[C@H](Cc1ccccc1)NC(=O)CNC(=O)CNC(=O)CCCCCN1C(=O)CC(*)C1=O]

in particular embodiments,

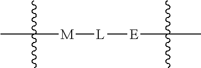

is selected from the following structures:

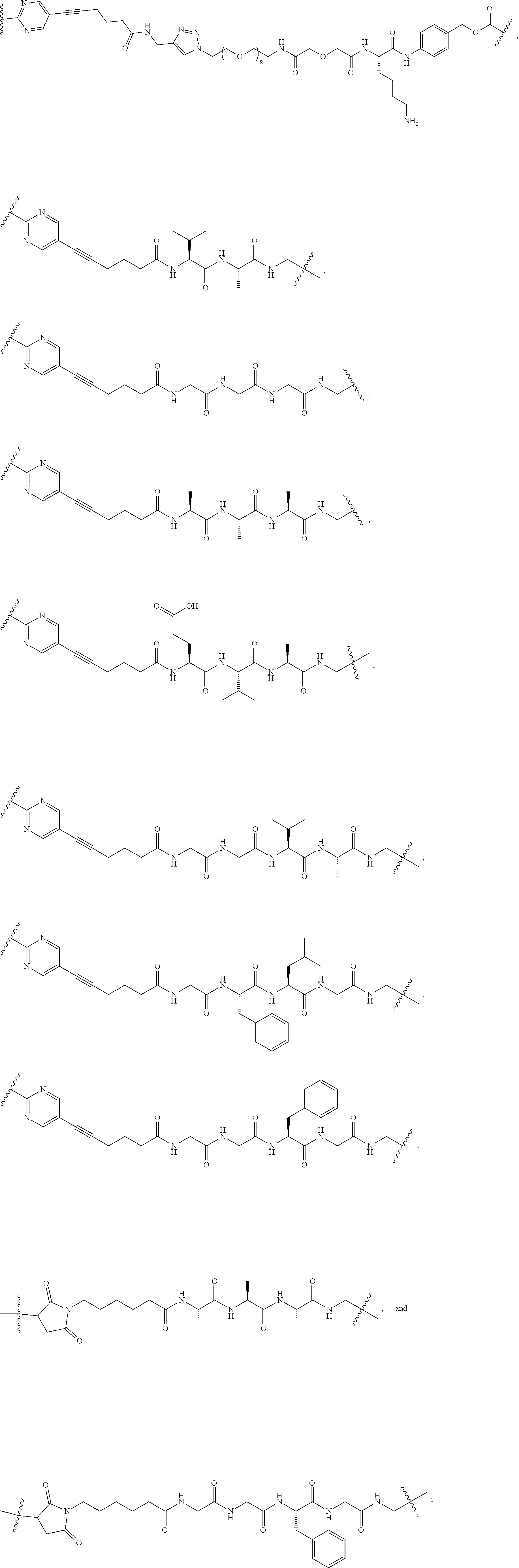

and
in particular embodiments,

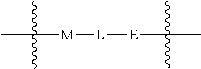

is selected from the following structures:

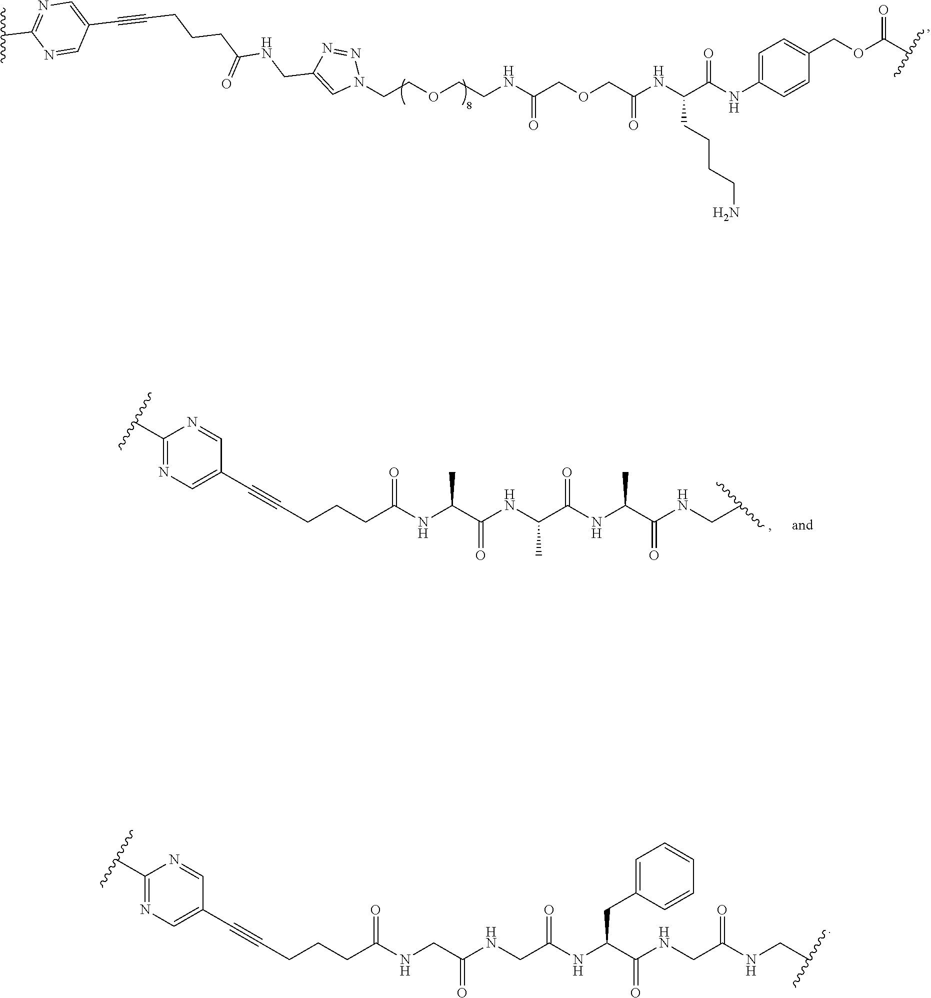

In some embodiments, the cytotoxic drug is selected from a tubulin inhibitor, a DNA intercalator, a DNA topoisomerase inhibitor, and an RNA polymerase inhibitor. In some embodiments, the tubulin inhibitor is an auristatin compound or a maytansine compound. In some embodiments, the DNA intercalator is pyrrolobenzodiazepine (PBD. In some embodiments, the DNA topoisomerase inhibitor is a topoisomerase I inhibitor (for example, camptothecin, hydroxycamptothecin, 9-aminocamptothecin, SN-38, irinotecan, topotecan, belotecan, or rubitecan) or a topoisomerase II inhibitor (for example, doxorubicin, PNU-159682, duocarmycin, daunorubicin, mitoxantrone, podophyllotoxin, or etoposide). In some embodiments, the RNA polymerase inhibitor is α-amanitin, or a pharmaceutically acceptable salt, an ester or an analog thereof. In some embodiments, the cytotoxic drug is selected from the following formula I and formula II:

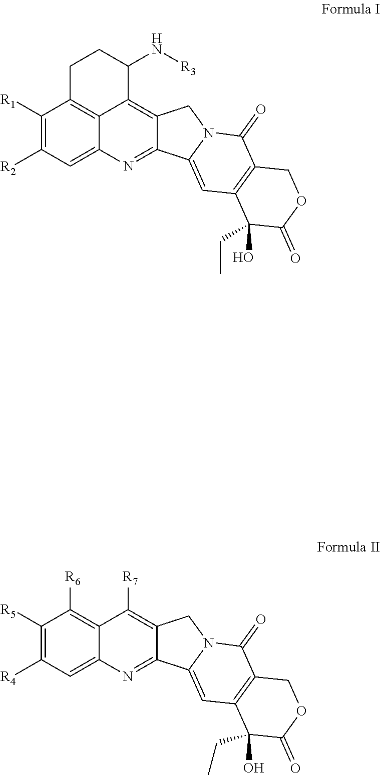

wherein R₁ and R₂ are each independently selected from C₁₋₆alkyl and halogen;
R₃ is selected from H and —CO—CH₂OH;
R₄ and R₅ are each independently selected from H, halogen, and hydroxy; or R₄ and R₅ are taken together with the carbon atom to which they are attached to form a 5-6-membered oxygen-containing heterocyclic ring;
R₆ is selected from hydrogen or —C₁₋₄alkylene-NRₐR_{b}; and
R₇ is selected from hydrogen, C₁₋₆ alkyl, —C₁₋₄ alkylene-NRₐR_{b}, —C₁₋₄ alkylene-SiRₐR_{b}R_{c}, —SiRₐR_{b}R_{c}, —C₁₋₄ alkylene=N—ORₐ; wherein, Rₐ, R_{b} and R_{c} are independently selected from H, C₁₋₆ alkyl, —SO₂—C₁₋₆ alkyl, and —CO—C₁₋₆ alkyl at each occurrence; wherein optionally Rₐ and R_{b} connected to the associated atoms form a 5-6 membered nitrogen containing heterocyclic ring.

In some embodiments, the cytotoxic drug is selected from the following compounds:

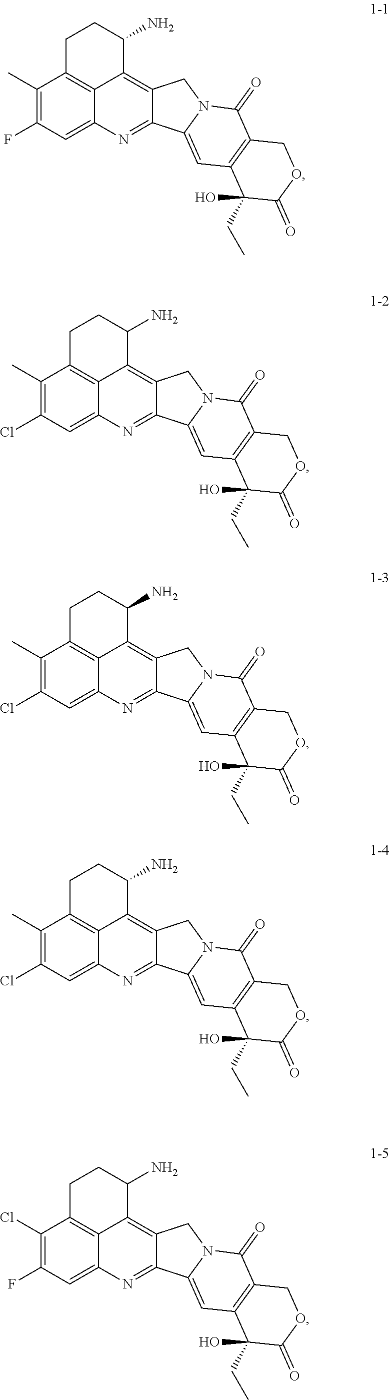 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

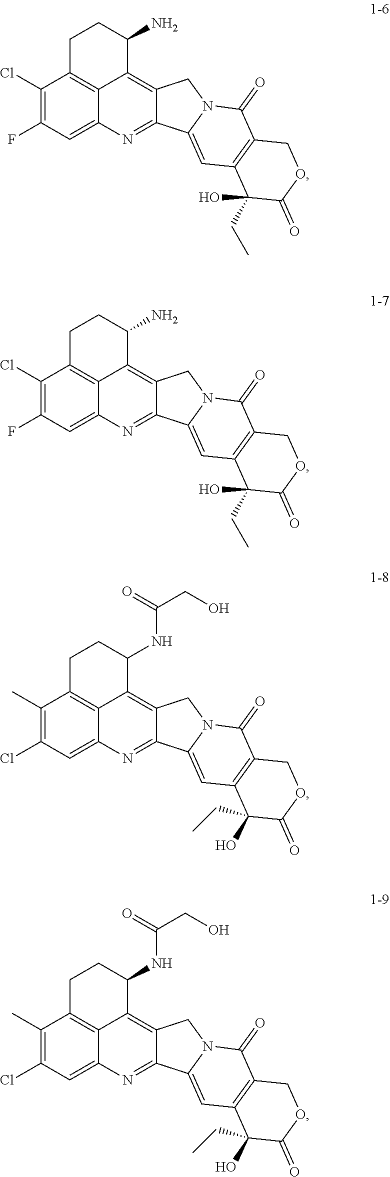 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3]

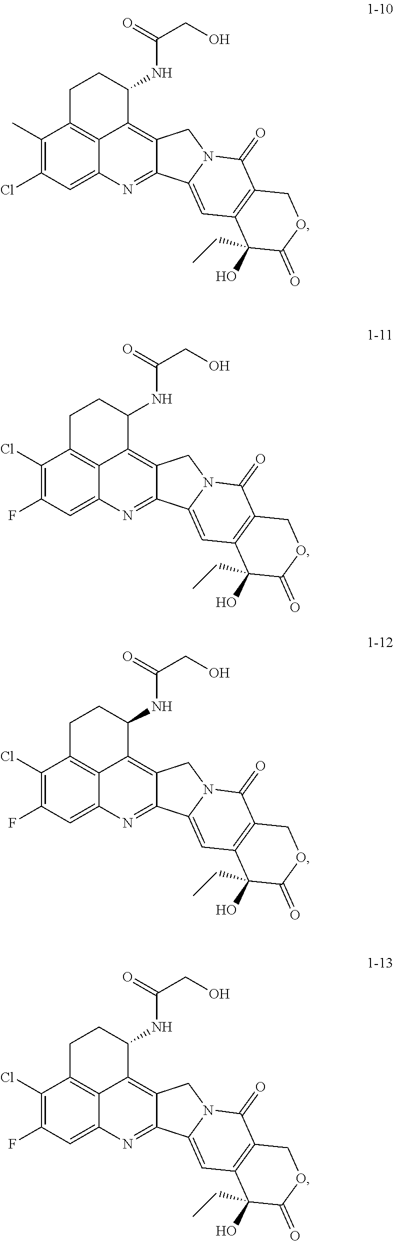 [SMILES: CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3]

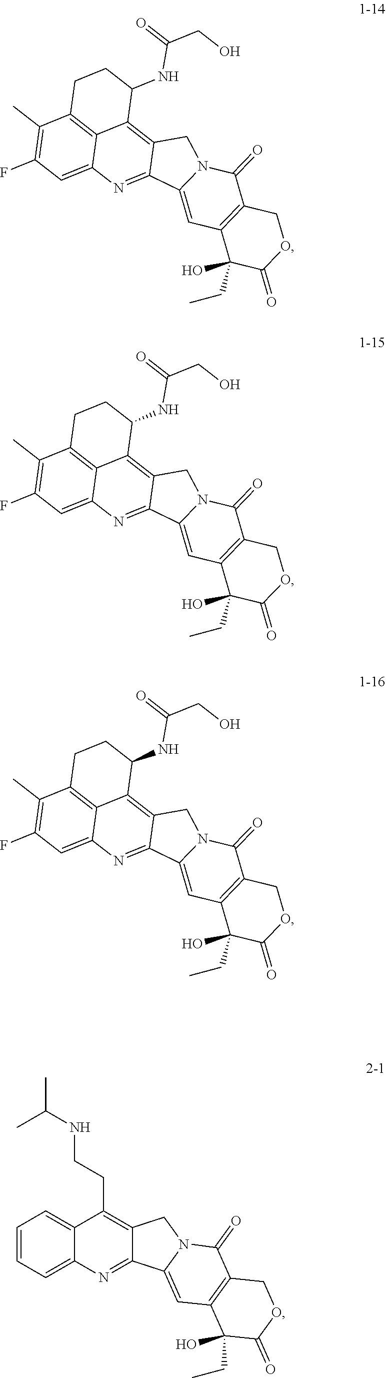 [SMILES: CC[C@@]1(O)C(=O)OCc2c1cc1n(c2=O)Cc2c-1nc1cc(F)c(C)c3c1c2C(NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1cc(F)c(C)c3c1c2[C@H](NC(=O)CO)CC3 ; CC[C@@]1(O)C(=O)OCc2c1cc1n(c2=O)Cc2c-1nc1ccccc1c2CCNC(C)C]

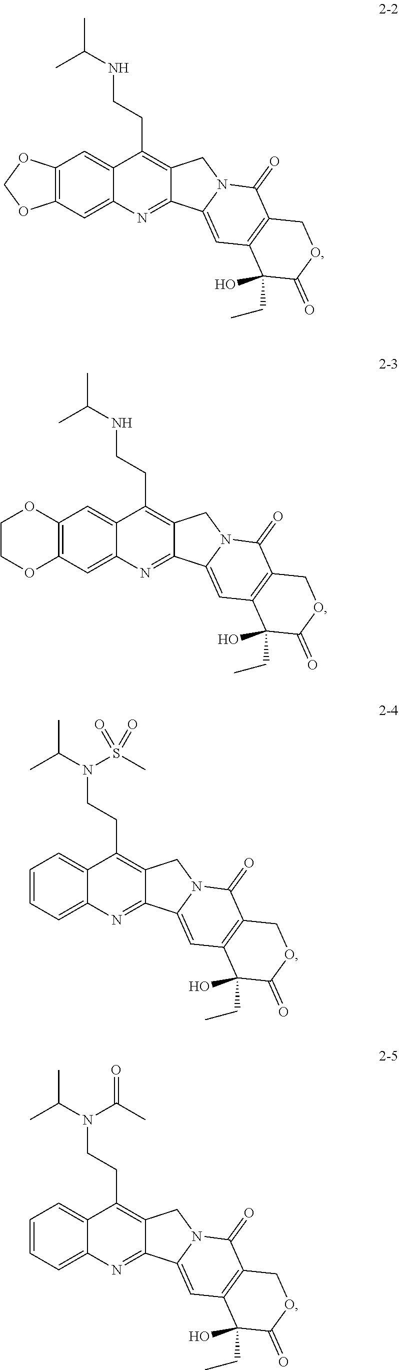 [SMILES: CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C]

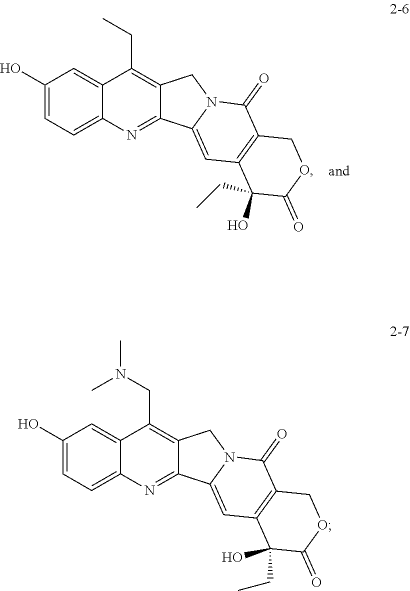 [SMILES: CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2c-1nc1ccc(O)cc1c2CN(C)C]

in particular embodiments, the cytotoxic drug is selected from the following compounds:

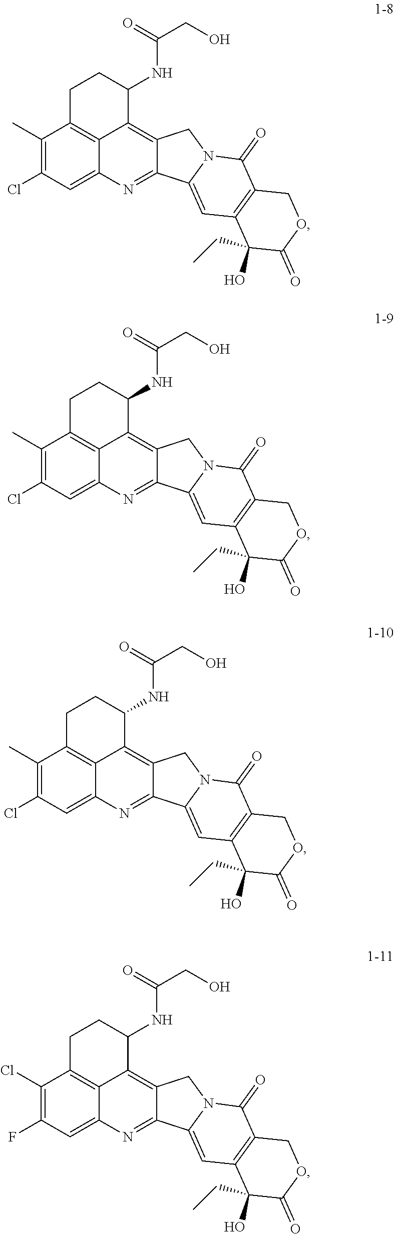 [SMILES: CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3]

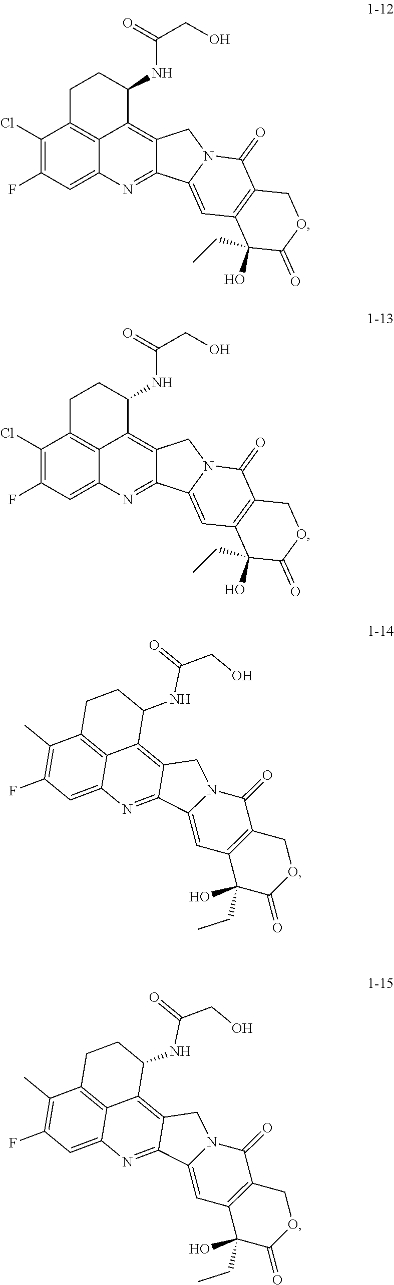 [SMILES: CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2C(NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

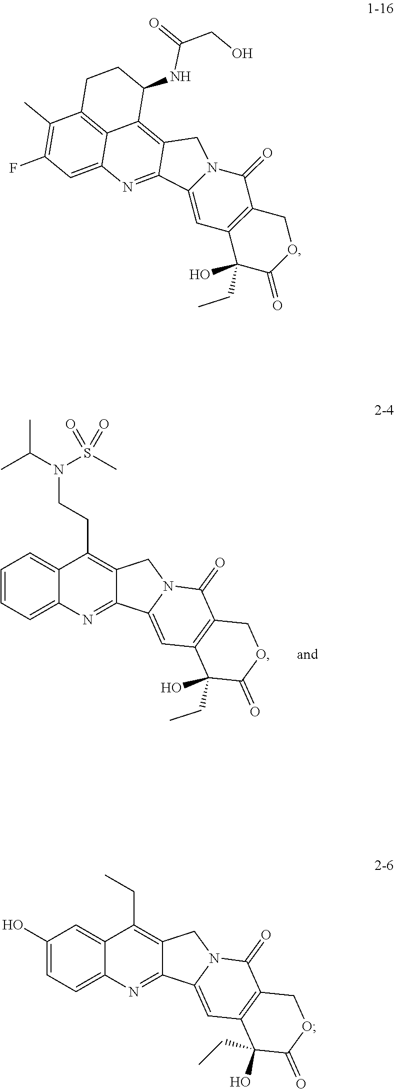 [SMILES: CC[C@@]1(O)C(=O)OCc2c1cc1n(c2=O)Cc2c-1nc1cc(F)c(C)c3c1c2[C@H](NC(=O)CO)CC3 ; CC[C@@]1(O)C(=O)OCc2c1cc1n(c2=O)Cc2c-1nc1ccccc1c2CCN(C(C)C)S(C)(=O)=O ; "and" ; CCc1c2c(nc3ccc(O)cc13)-c1cc3c(c(=O)n1C2)COC(=O)[C@]3(O)CC]

in particular embodiments, the cytotoxic drug is selected from the following compounds:

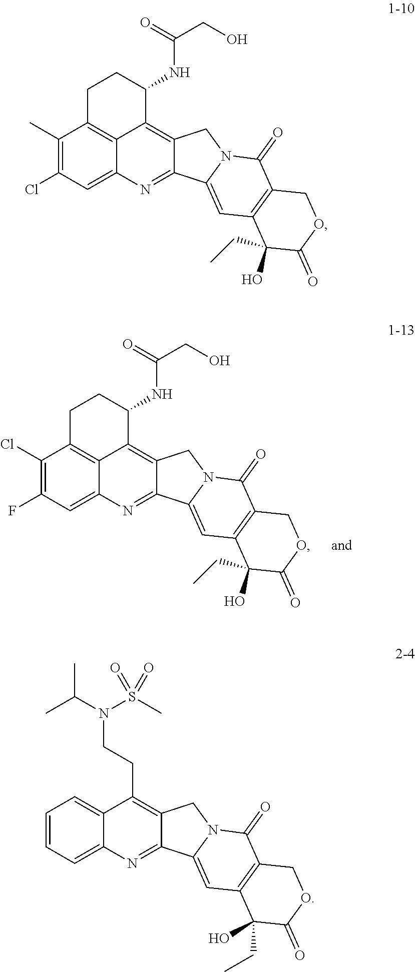 [SMILES: CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCN(C(C)C)S(C)(=O)=O]

In some embodiments, D is selected from the following structures:

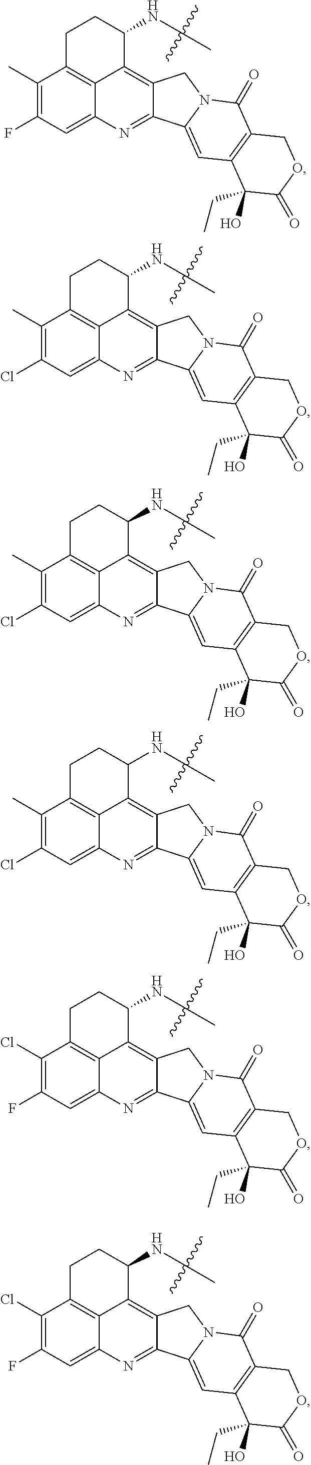 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC ; *N[C@H]1CCc2c(Cl)c(F)cc3nc4c(c1c23)Cn1c-4cc2c(c1=O)COC(=O)[C@]2(O)CC ; *N[C@@H]1CCc2c(Cl)c(F)cc3nc4c(c1c23)Cn1c-4cc2c(c1=O)COC(=O)[C@]2(O)CC]

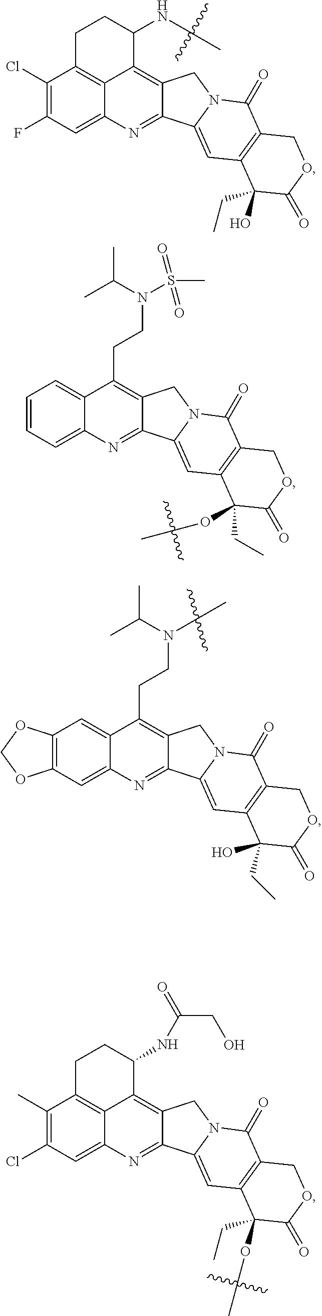 [SMILES: *NC1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O ; *N(CCc1c2c(nc3cc4c(cc13)OCO4)-c1cc3c(c(=O)n1C2)COC(=O)[C@]3(O)CC)C(C)C ; *O[C@]1(CC)C(=O)OCc2c1cc1n(c2=O)Cc2c-1nc1cc(Cl)c(C)c3c1c2[C@@H](NC(=O)CO)CC3]

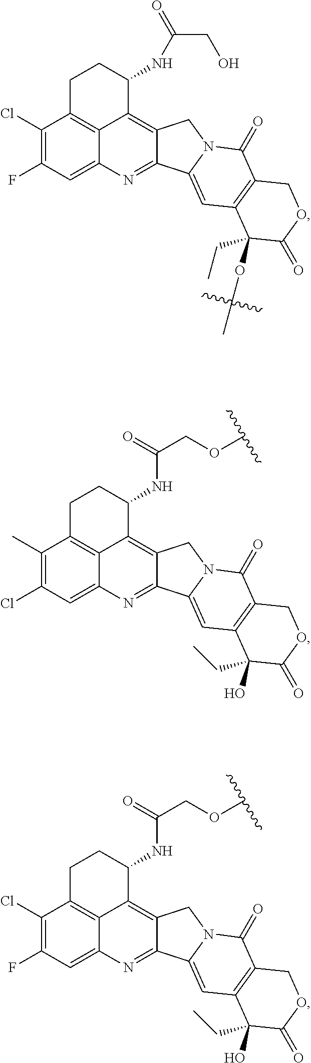 [SMILES: *O[C@]1(CC)C(=O)OCc2c1cc1n(c2=O)Cc2c-1nc1cc(F)c(Cl)c3c1c2[C@@H](NC(=O)CO)CC3 ; *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC]

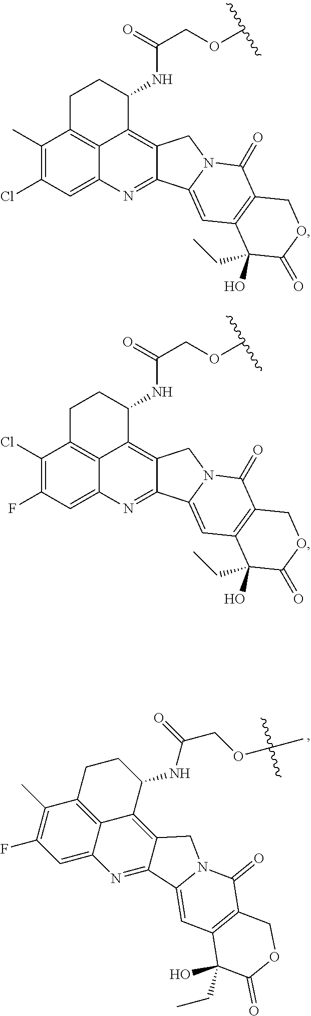 [SMILES: *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC]

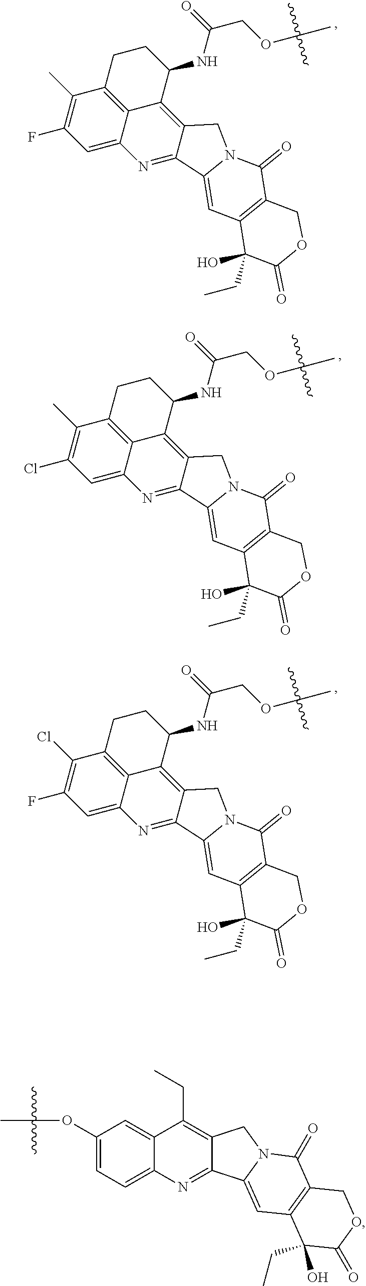

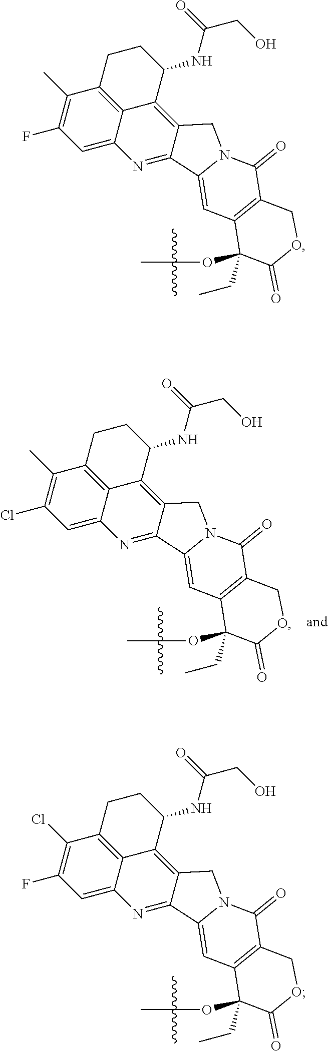 [SMILES: *O[C@]1(CC)C(=O)OCc2c1cc1n(c2=O)Cc2c-1nc1cc(F)c(C)c3c1c2[C@@H](NC(=O)CO)CC3 ; *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3]

in particular embodiments, D is selected from the following structures:

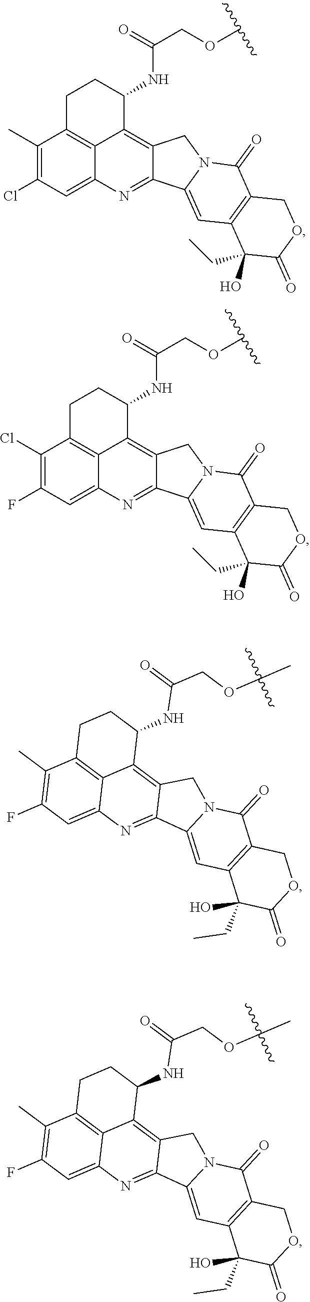 [SMILES: *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *OCC(=O)N[C@@H]1CCc2c(C)c(F)cc3nc4c(c1c23)Cn1c-4cc2c(c1=O)COC(=O)[C@]2(O)CC]

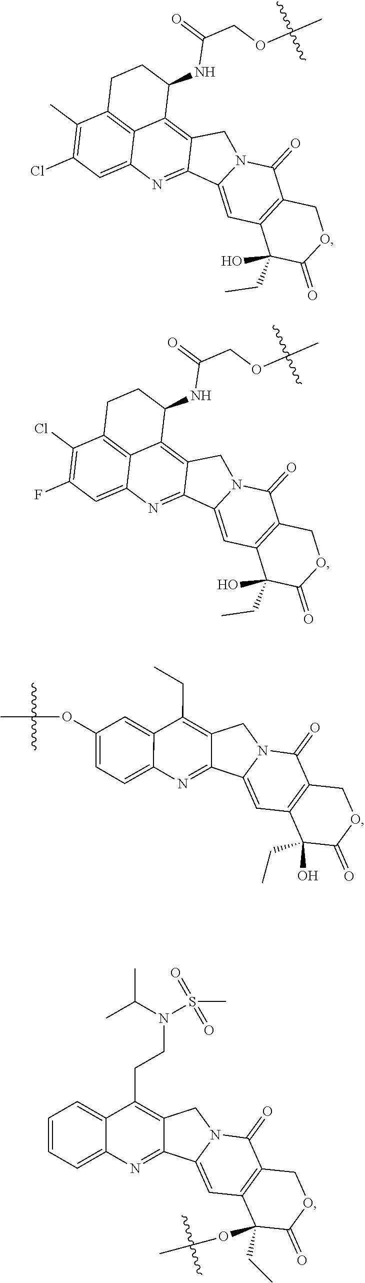 [SMILES: *OCC(=O)N[C@@H]1CCc2c(C)c(Cl)cc3nc4c(c1c23)Cn1c-4cc2c(c1=O)COC(=O)[C@]2(O)CC ; *OCC(=O)N[C@@H]1CCc2c(Cl)c(F)cc3nc4c(c1c23)Cn1c-4cc2c(c1=O)COC(=O)[C@]2(O)CC ; *Oc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O]

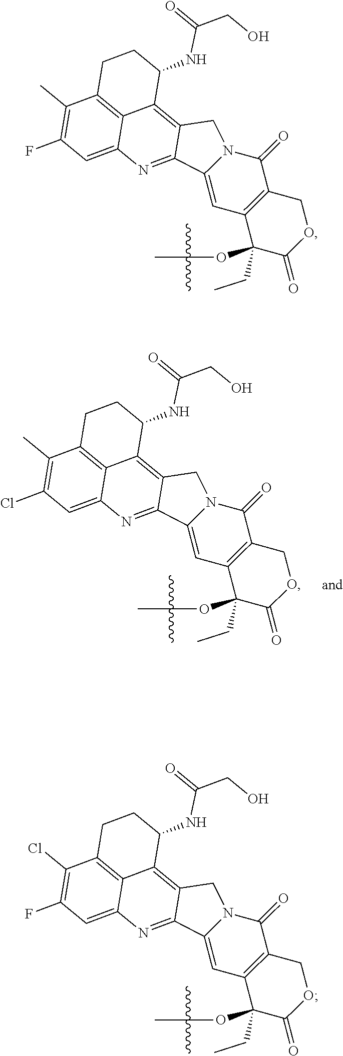 [SMILES: *O[C@]1(CC)C(=O)OCc2c1cc1n(c2=O)Cc2c-1nc1cc(F)c(C)c3c1c2[C@@H](NC(=O)CO)CC3 ; *O[C@]1(CC)C(=O)OCc2c1cc1n(c2=O)Cc2c-1nc1cc(Cl)c(C)c3c1c2[C@@H](NC(=O)CO)CC3 ; "and" ; *O[C@]1(CC)C(=O)OCc2c1cc1n(c2=O)Cc2c-1nc1cc(F)c(Cl)c3c1c2[C@@H](NC(=O)CO)CC3]

in particular embodiments, D is selected from the following structures:

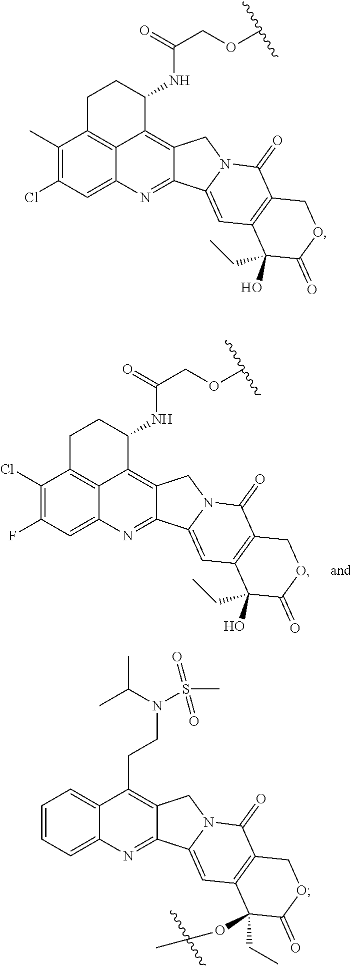 [SMILES: *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O]

and
in particular embodiments, D is selected from the following structures:

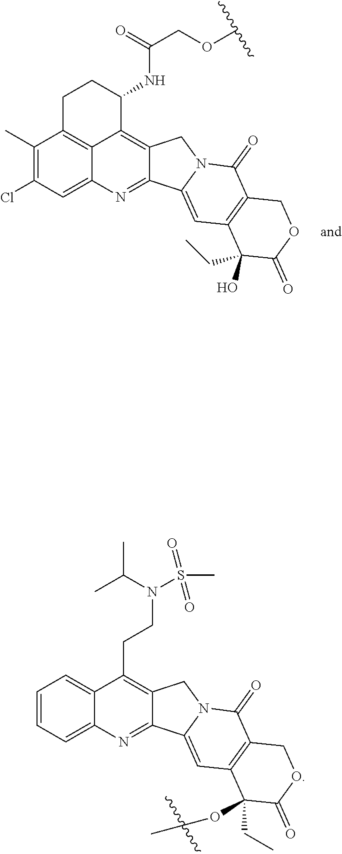 [SMILES: *OCC(=O)N[C@H]1CCc2c(C)c(Cl)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O]

In some embodiments, the antibody-drug conjugate is selected from ADC A-01 to ADC A-34 and ADC B-01 to ADC B-07 as shown below:

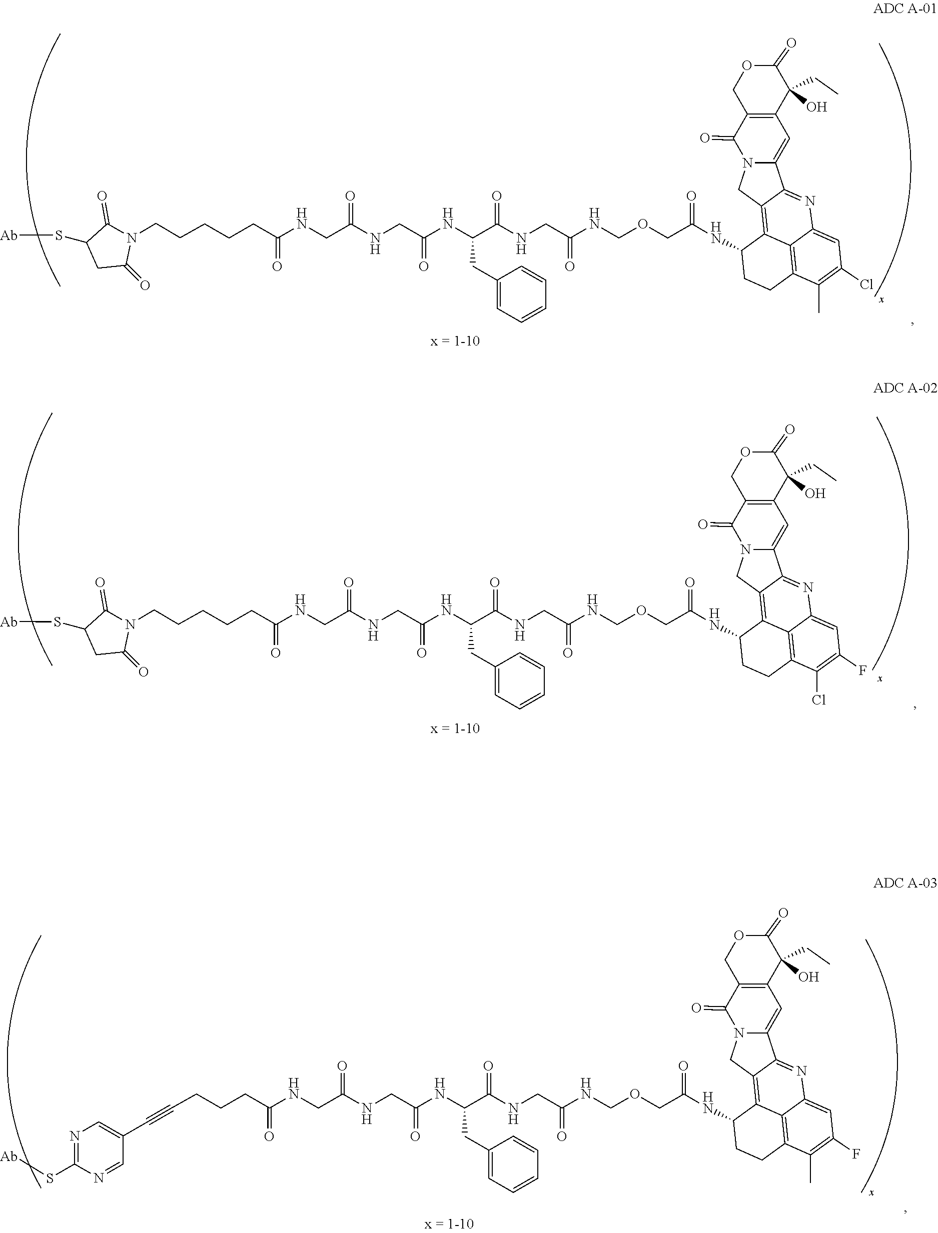

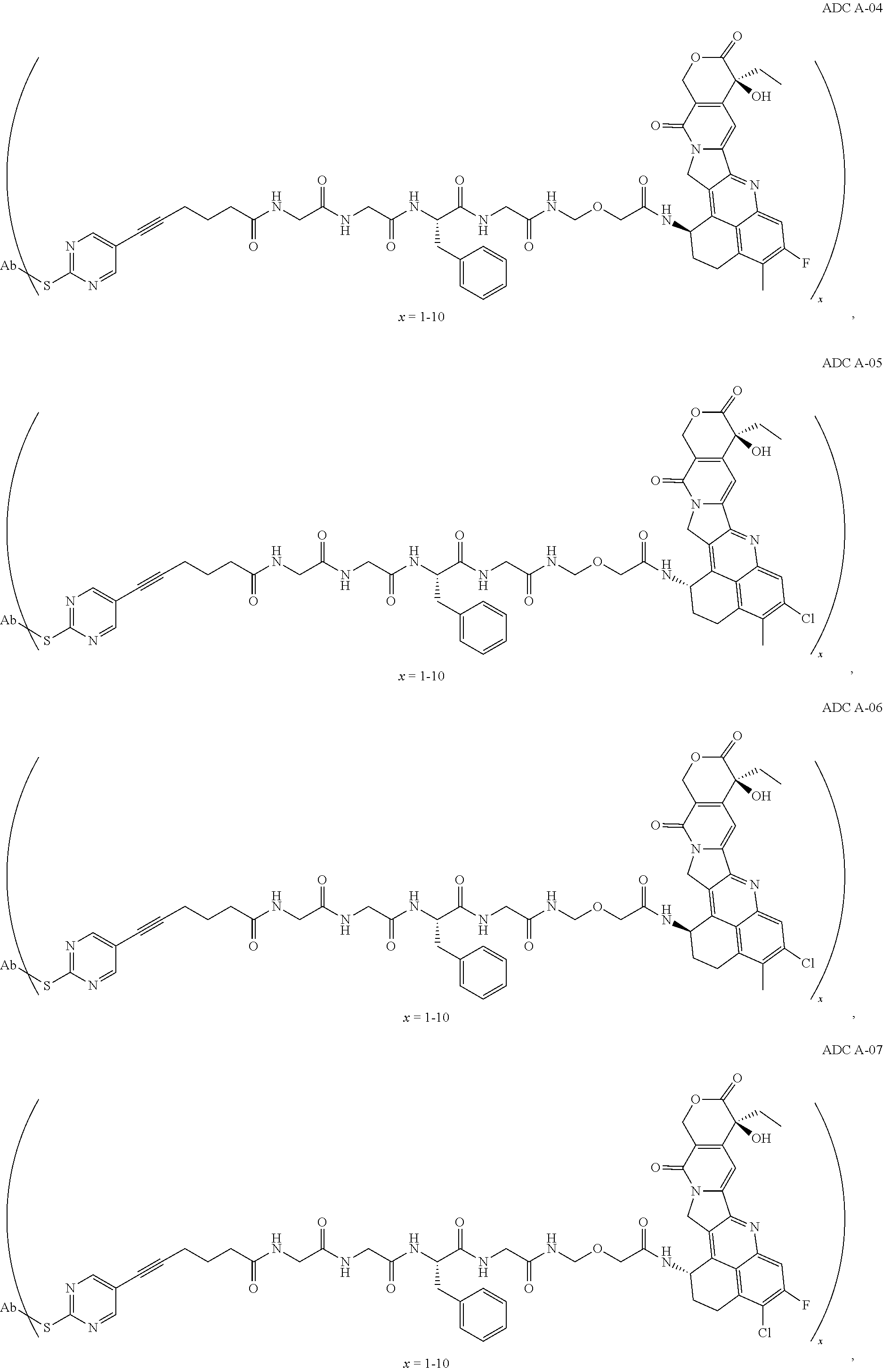

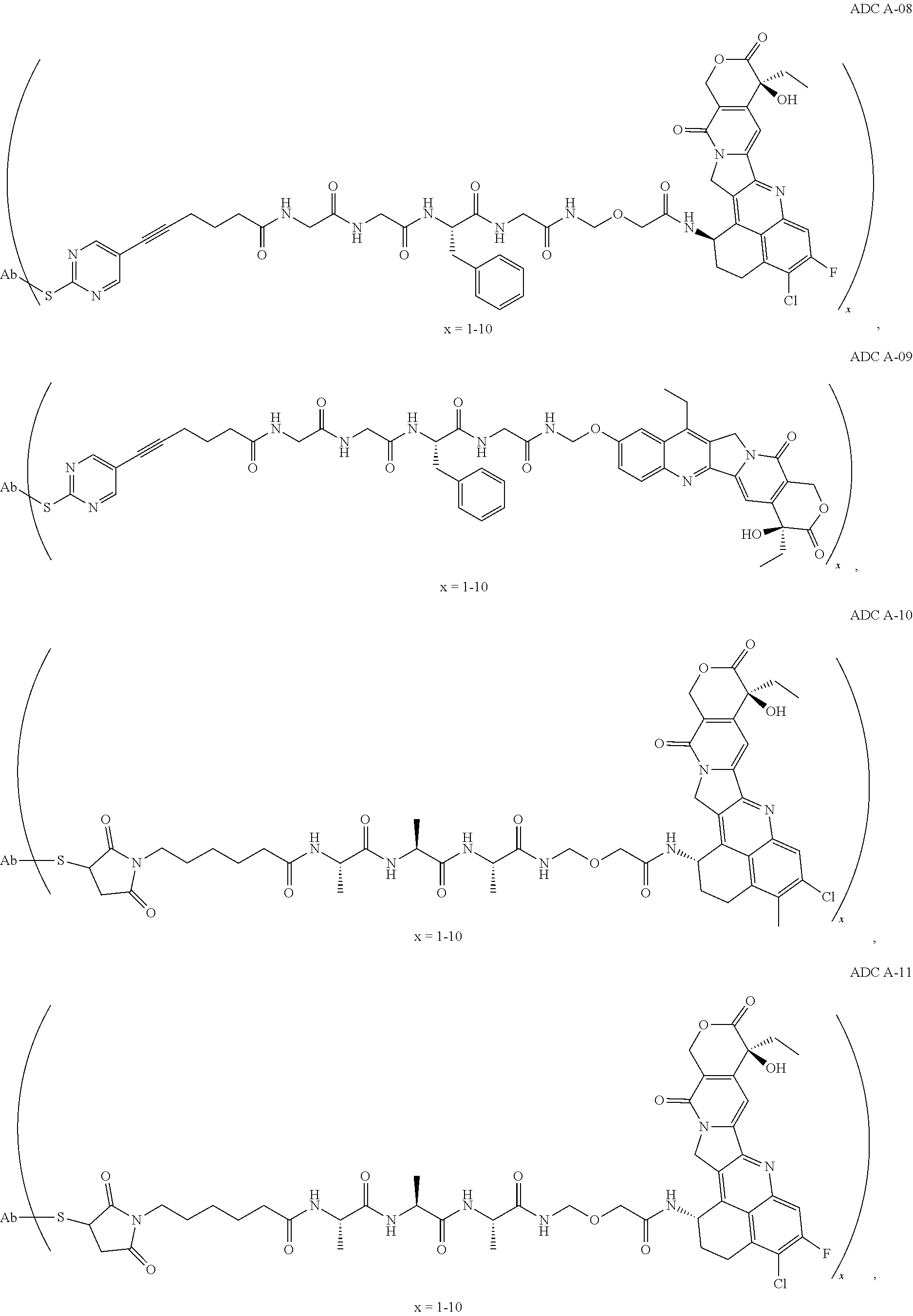

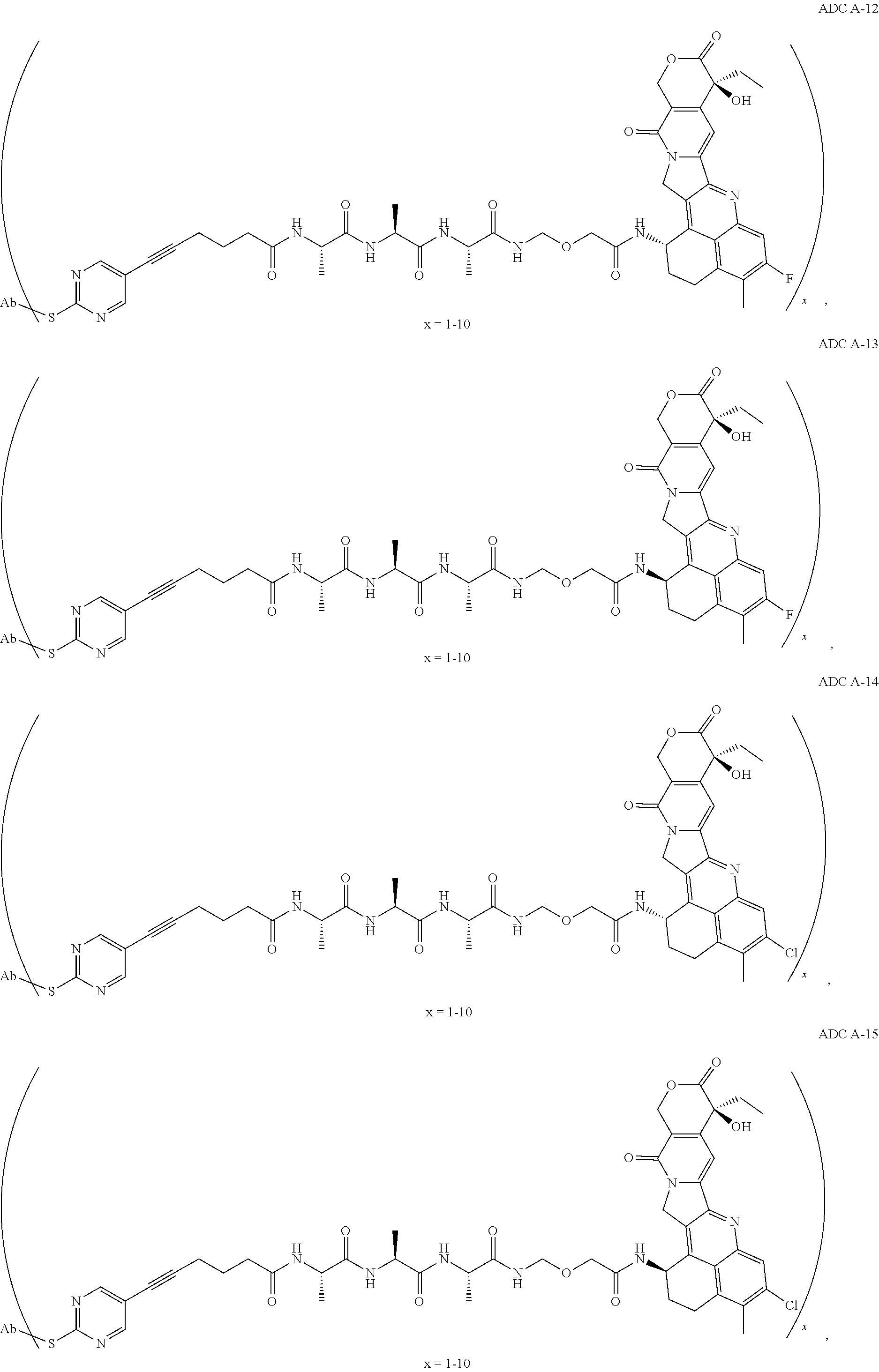

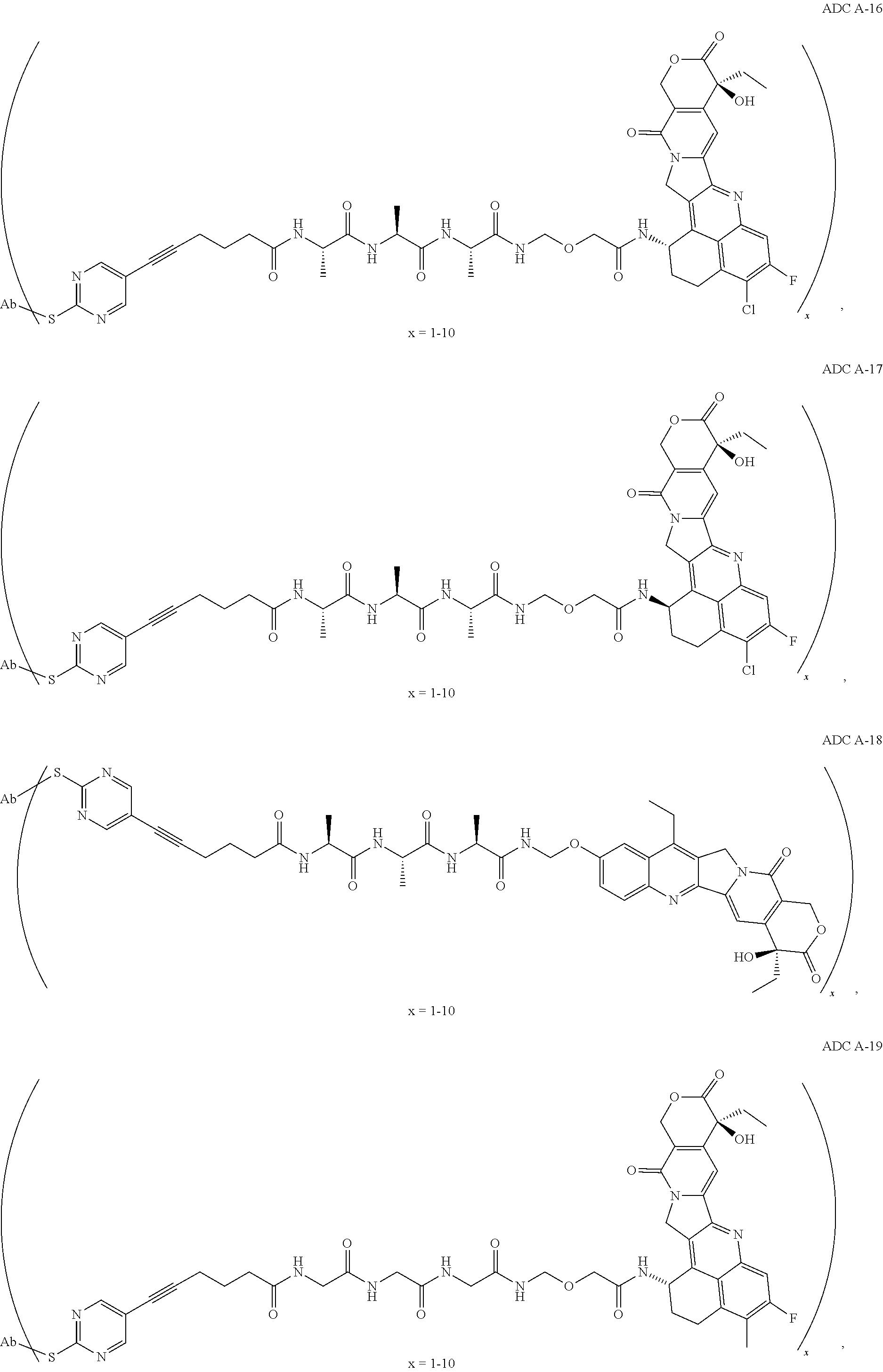

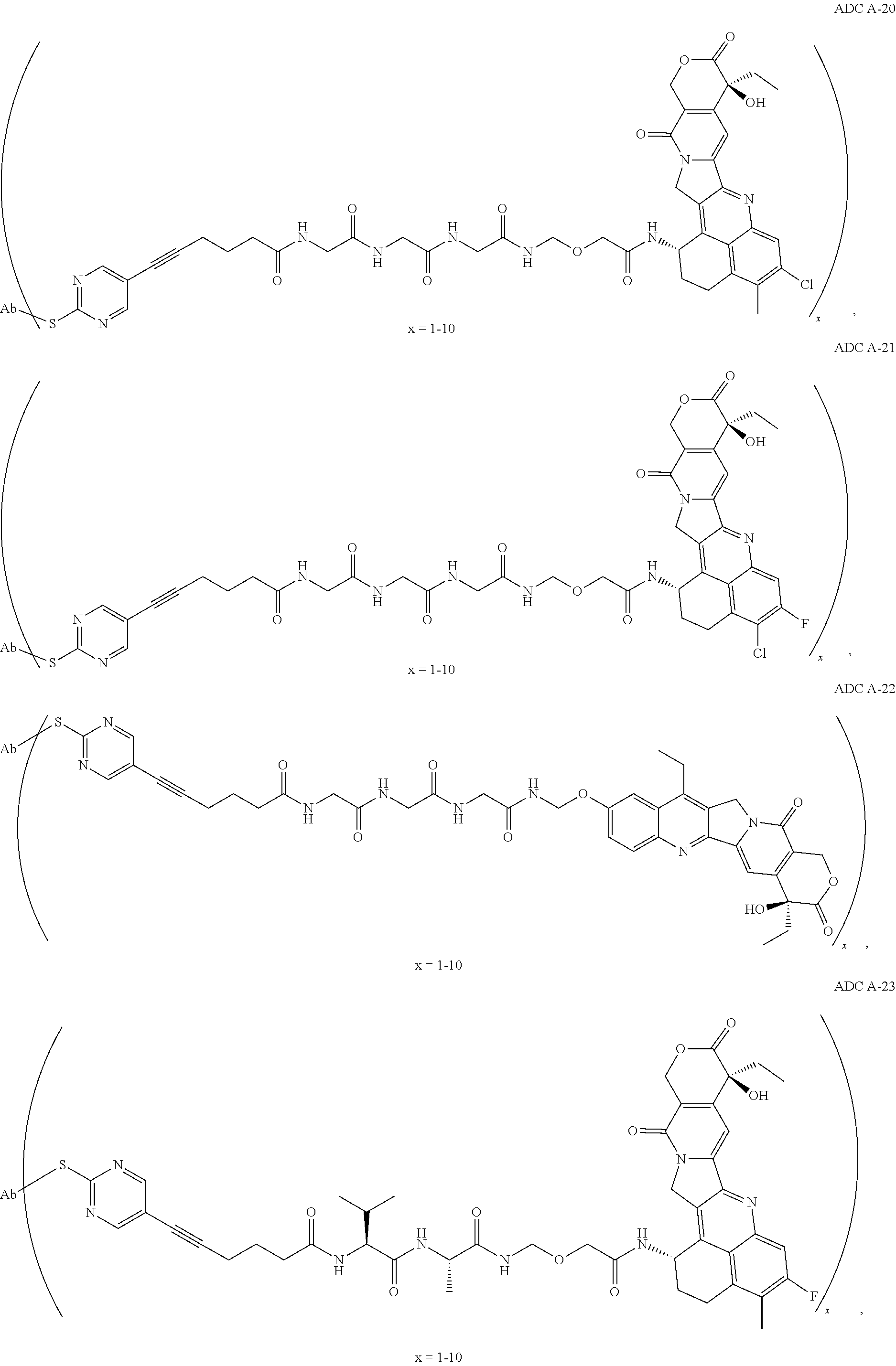

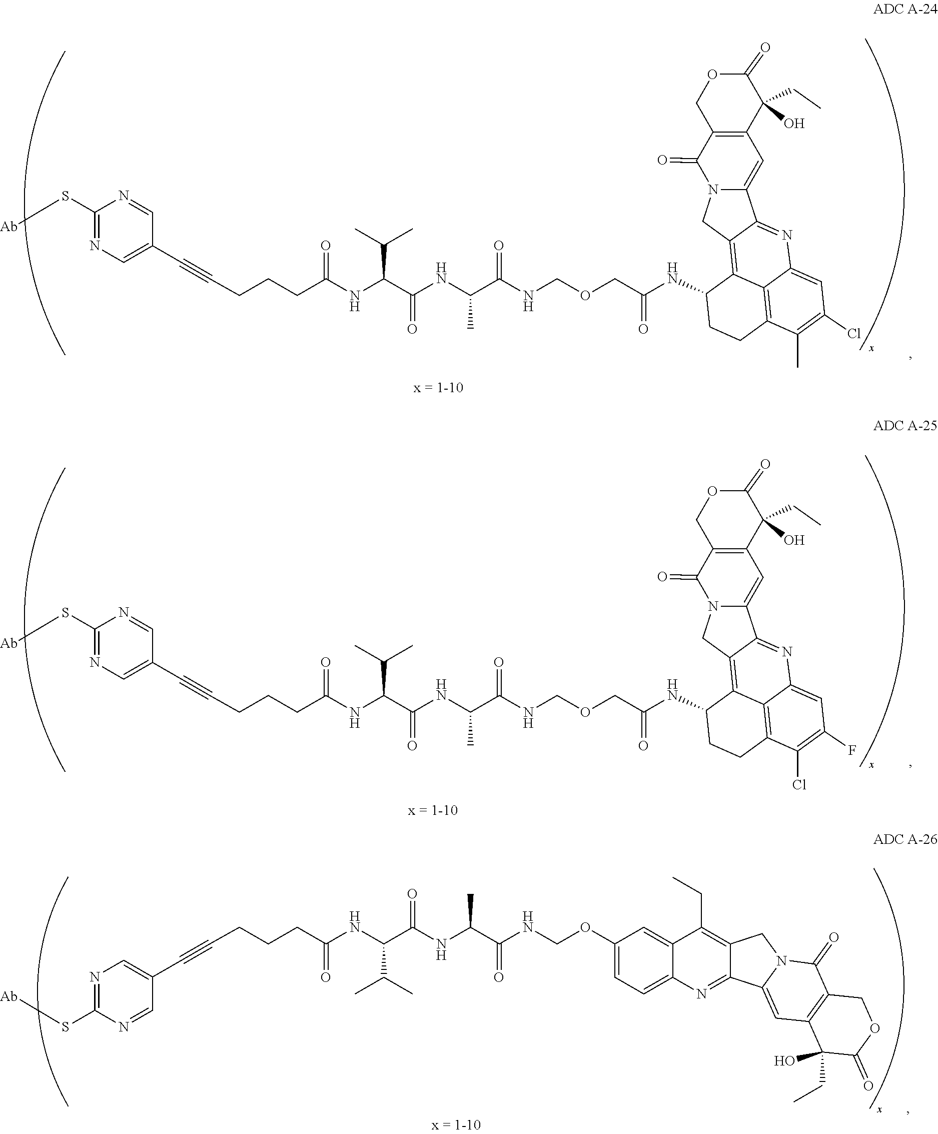

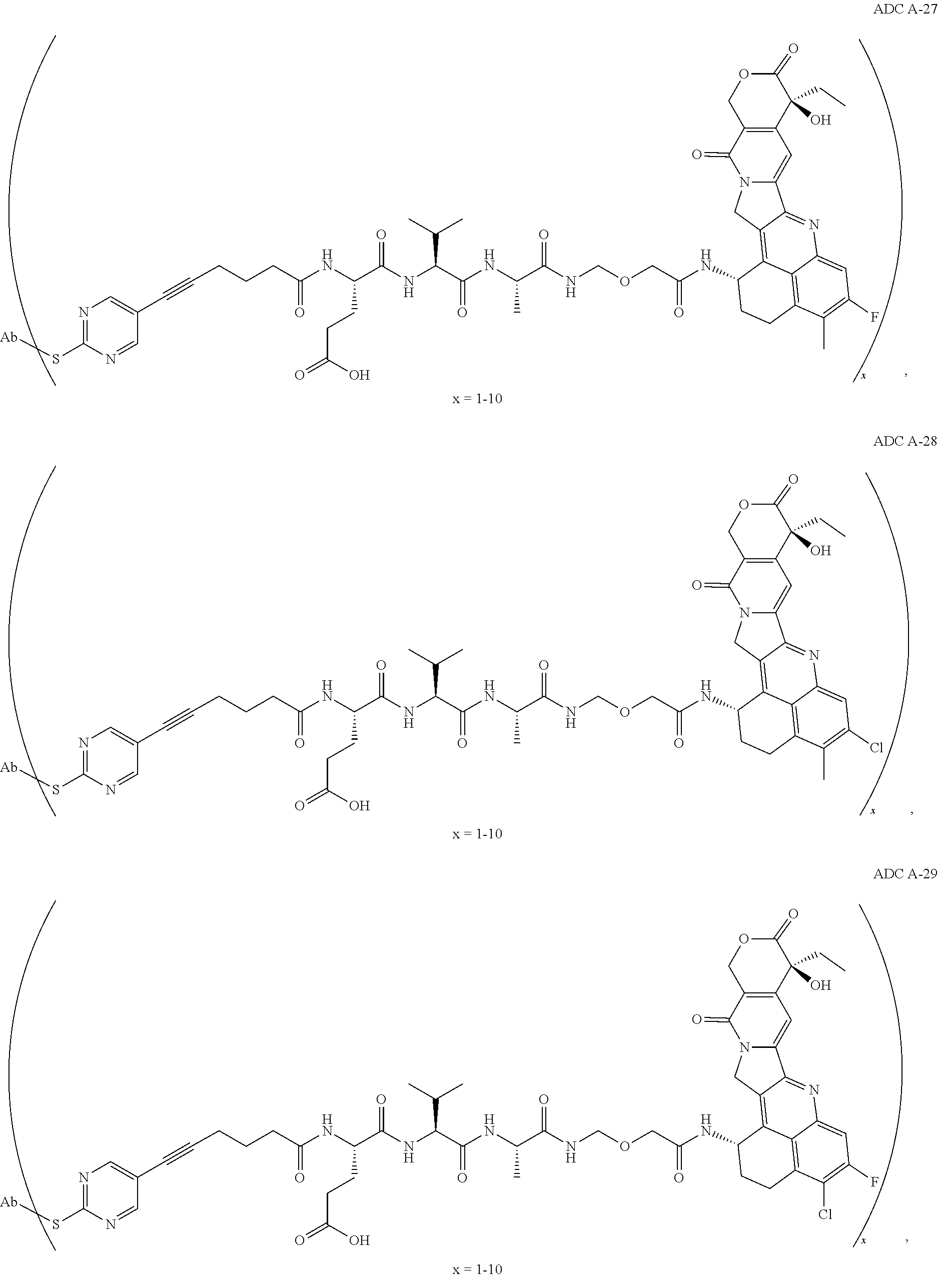

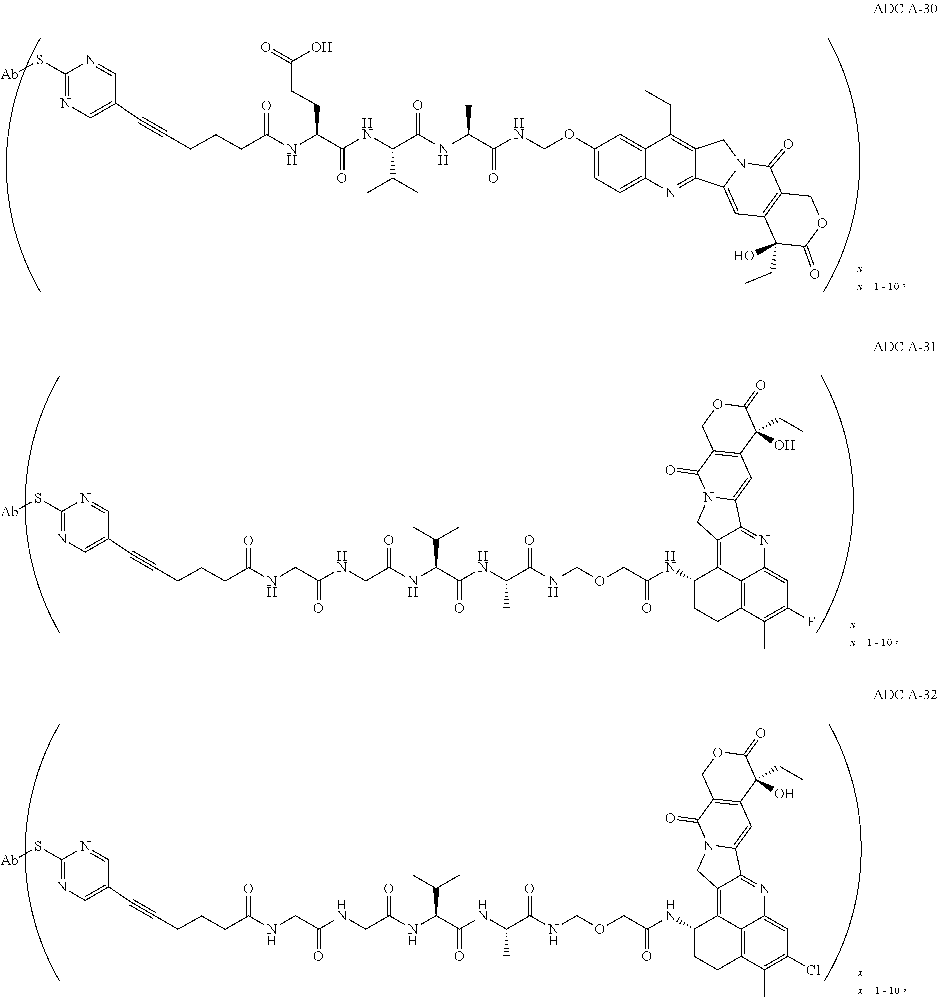

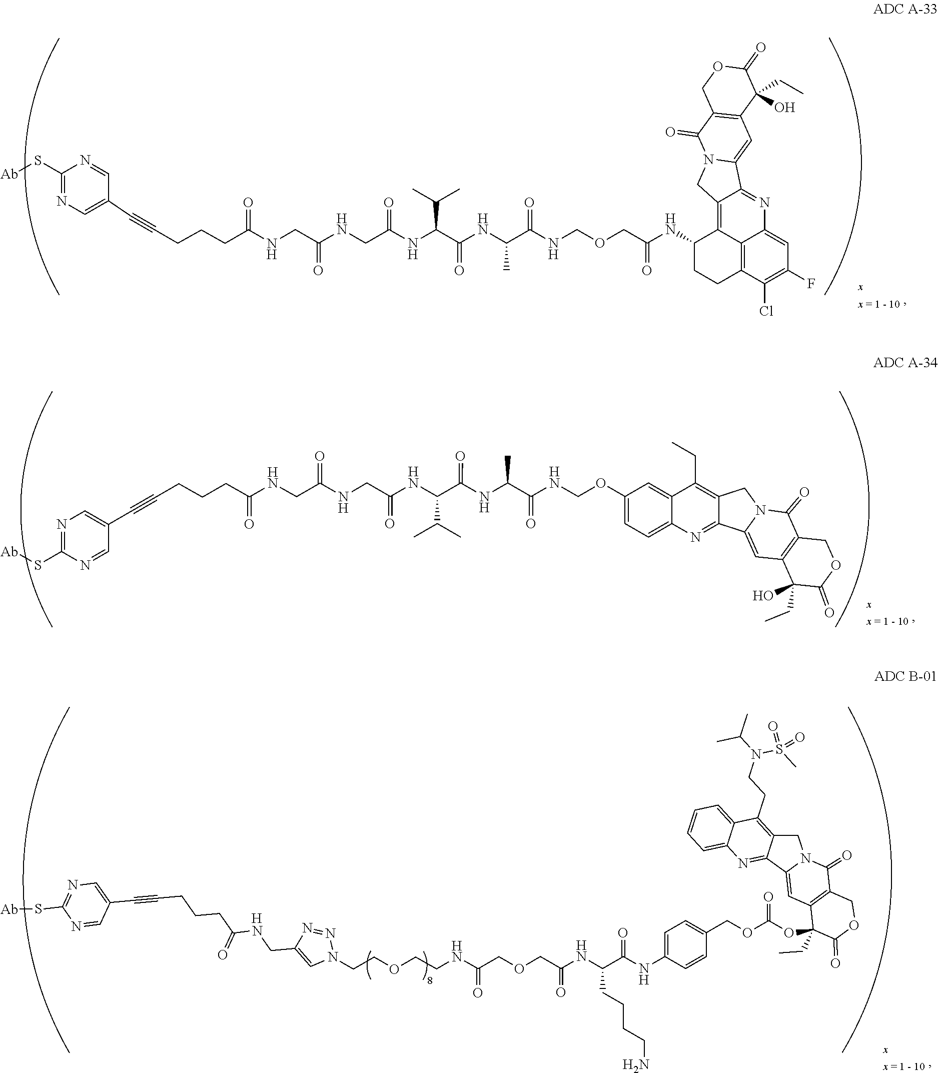

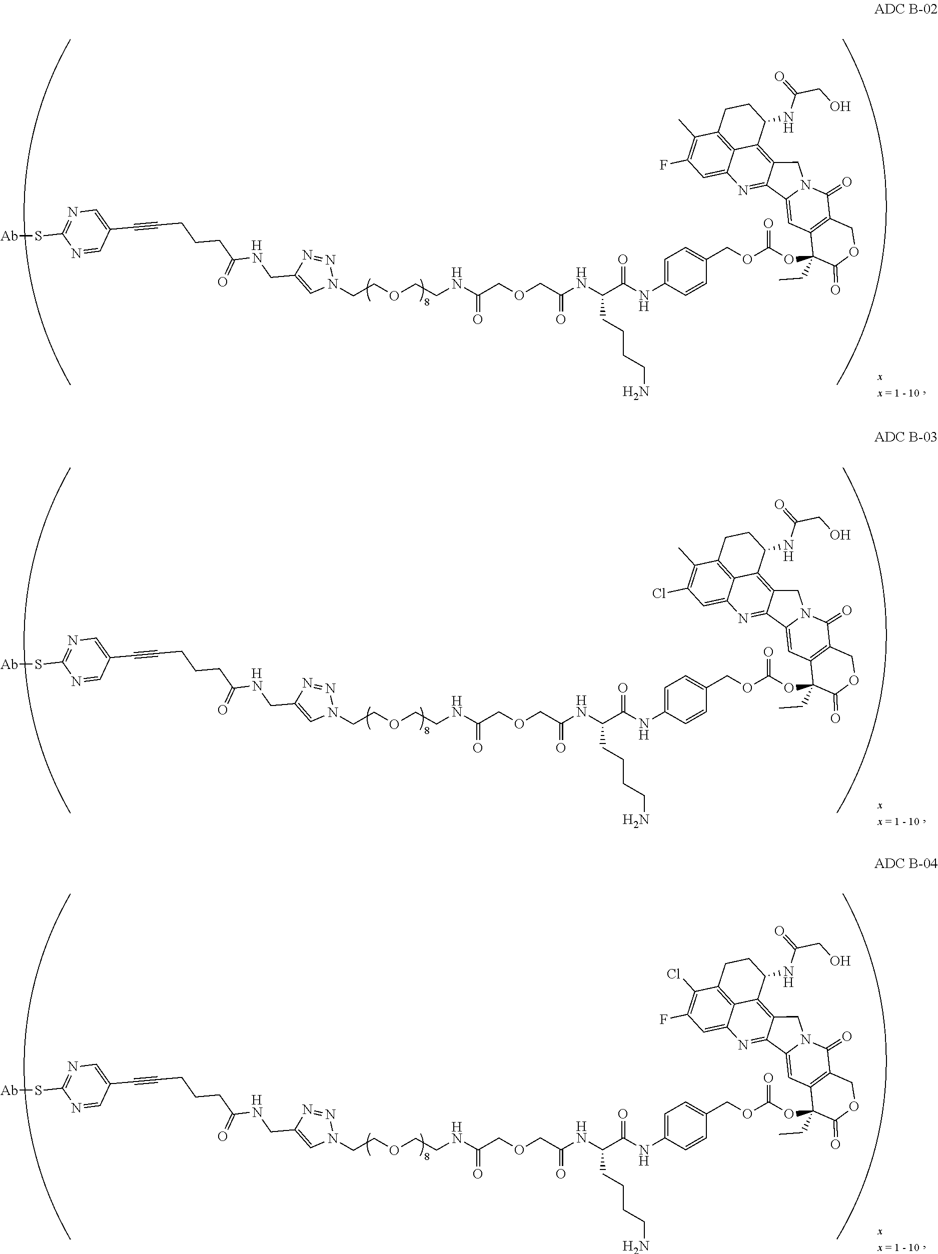

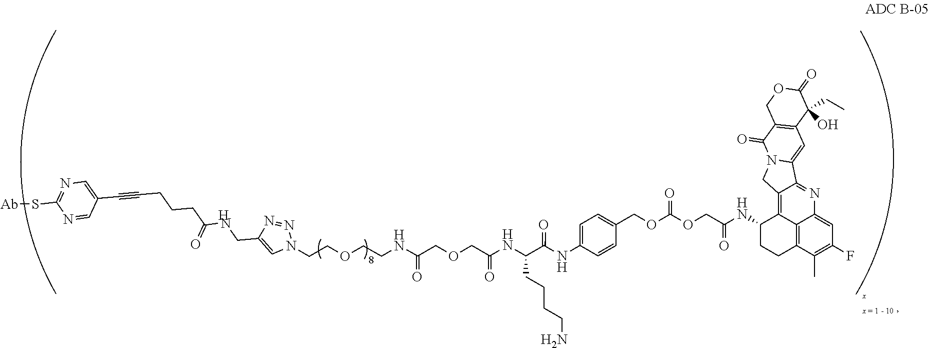

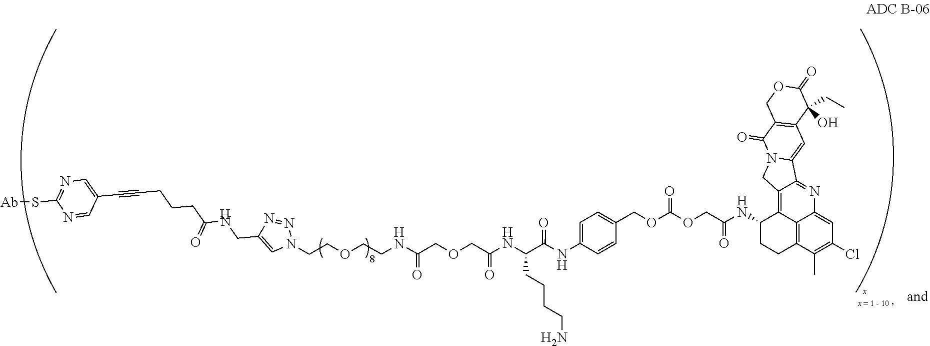

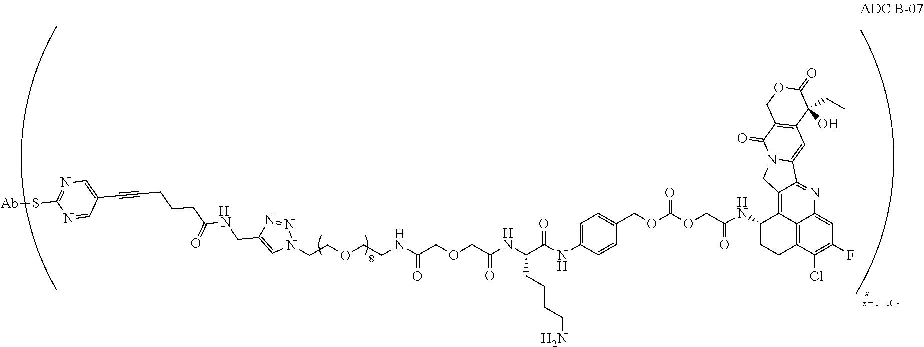

wherein Ab-(S— in each antibody-drug conjugate represents the antibody or the antigen-binding fragment thereof of any one above; and
wherein

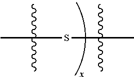

represents a specific mode for connecting the sulfhydryl in the antibody or the antigen-binding fragment thereof with the linker.

Also provided is a composition comprising one or more antibody-drug conjugates of the disclosure, wherein a DAR value of the composition is 1-10, for example: 1, 2, 3, 4, 5, 6, 7, 8, 9, 10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10, in particular embodiments, 3-8, for example, 3.0-3.5, 3.0-4.0, 3.0-4.5, 3.0-5.0, 6.0-6.5, 6.0-7.0, 6.0-7.5, 6.0-8.0, 6.0-8.5, 6.5-7.0, 6.5-7.5, 6.5-8.0, 6.5-8.5, 7.0-7.5, 7.0-8.0, or 7.5-8.0. In some embodiments, the DAR value of the composition is about 6.0 to 10.0, for example, about 6.0, about 6.01, about 6.02, about 6.03, about 6.04, about 6.05, about 6.06, about 6.07, about 6.08, about 6.09, about 6.1, about 6.11, about 6.12, about 6.13, about 6.14, about 6.15, about 6.16, about 6.17, about 6.18, about 6.19, about 6.2, about 6.21, about 6.22, about 6.23, about 6.24, about 6.25, about 6.26, about 6.27, about 6.28, about 6.29, about 6.3, about 6.31, about 6.32, about 6.33, about 6.34, about 6.35, about 6.36, about 6.37, about 6.38, about 6.39, about 6.4, about 6.41, about 6.42, about 6.43, about 6.44, about 6.45, about 6.46, about 6.47, about 6.48, about 6.49, about 6.5, about 6.51, about 6.52, about 6.53, about 6.54, about 6.55, about 6.56, about 6.57, about 6.58, about 6.59, about 6.6, about 6.61, about 6.62, about 6.63, about 6.64, about 6.65, about 6.66, about 6.67, about 6.68, about 6.69, about 6.7, about 6.71, about 6.72, about 6.73, about 6.74, about 6.75, about 6.76, about 6.77, about 6.78, about 6.79, about 6.8, about 6.81, about 6.82, about 6.83, about 6.84, about 6.85, about 6.86, about 6.87, about 6.88, about 6.89, about 6.9, about 6.91, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, about 7.1, about 7.11, about 7.12, about 7.13, about 7.14, about 7.15, about 7.16, about 7.17, about 7.18, about 7.19, about 7.2, about 7.21, about 7.22, about 7.23, about 7.24, about 7.25, about 7.26, about 7.27, about 7.28, about 7.29, about 7.3, about 7.31, about 7.32, about 7.33, about 7.34, about 7.35, about 7.36, about 7.37, about 7.38, about 7.39, about 7.4, about 7.41, about 7.42, about 7.43, about 7.44, about 7.45, about 7.46, about 7.47, about 7.48, about 7.49, about 7.5, about 7.51, about 7.52, about 7.53, about 7.54, about 7.55, about 7.56, about 7.57, about 7.58, about 7.59, about 7.6, about 7.61, about 7.62, about 7.63, about 7.64, about 7.65, about 7.66, about 7.67, about 7.68, about 7.69, about 7.7, about 7.71, about 7.72, about 7.73, about 7.74, about 7.75, about 7.76, about 7.77, about 7.78, about 7.79, about 7.8, about 7.81, about 7.82, about 7.83, about 7.84, about 7.85, about 7.86, about 7.87, about 7.88, about 7.89, about 7.9, about 7.91, about 7.92, about 7.93, about 7.94, about 7.95, about 7.96, about 7.97, about 7.98, about 7.99, about 8.0, about 8.01, about 8.02, about 8.03, about 8.04, about 8.05, about 8.06, about 8.07, about 8.08, about 8.09, about 8.1, about 8.11, about 8.12, about 8.13, about 8.14, about 8.15, about 8.16, about 8.17, about 8.18, about 8.19, about 8.2, about 8.21, about 8.22, about 8.23, about 8.24, about 8.25, about 8.26, about 8.27, about 8.28, about 8.29, about 8.3, about 8.31, about 8.32, about 8.33, about 8.34, about 8.35, about 8.36, about 8.37, about 8.38, about 8.39, about 8.4, about 8.41, about 8.42, about 8.43, about 8.44, about 8.45, about 8.46, about 8.47, about 8.48, about 8.49, about 8.5, about 8.51, about 8.52, about 8.53, about 8.54, about 8.55, about 8.56, about 8.57, about 8.58, about 8.59, about 8.6, about 8.61, about 8.62, about 8.63, about 8.64, about 8.65, about 8.66, about 8.67, about 8.68, about 8.69, about 8.7, about 8.71, about 8.72, about 8.73, about 8.74, about 8.75, about 8.76, about 8.77, about 8.78, about 8.79, about 8.8, about 8.81, about 8.82, about 8.83, about 8.84, about 8.85, about 8.86, about 8.87, about 8.88, about 8.89, about 8.9, about 8.91, about 8.92, about 8.93, about 8.94, about 8.95, about 8.96, about 8.97, about 8.98, about 8.99, about 9.0, about 6.0-8.0, about 6.0-8.5, about 6.0-9.0, about 6.0-9.5, about 6.0-10.0, about 6.5-8.0, about 6.5-8.5, about 6.5-9.0, about 6.5-9.5, about 6.5-10.0, about 7.0-8.0, about 7.0-8.5, about 7.0-9.0, about 7.0-9.5, about 7.0-10.0, about 7.5-8.0, about 7.5-8.5, about 7.5-9.0, about 7.5-9.5, about 7.5-10.0, about 8.0-8.5, about 8.0-9.0, about 8.0-9.5, about 8.0-10.0.

Also provided is a pharmaceutical composition comprising the antibody-drug conjugate of the disclosure or the composition of the disclosure, and one or more pharmaceutical excipients.

Also provided is use of the antibody-drug conjugate of the disclosure, the composition of the disclosure, or the pharmaceutical composition of the disclosure in the preparation of a medicament for preventing or treating a CDH3-mediated disease or condition. In some embodiments, the CDH3-mediated disease or condition is a tumor. In some embodiments, the tumor is a CDH3-positive tumor, for example, colorectal cancer, gastric cancer, lung cancer (e.g., non-small cell lung cancer), cervical cancer, pancreatic cancer, esophageal cancer, ovarian cancer, thyroid cancer, bladder cancer, endometrial cancer, breast cancer, liver cancer, prostate cancer, skin cancer, pharyngeal cancer, or bile duct cancer, or any combination thereof.

Also provided is a method of inhibiting the activity of CDH3 in cells, comprising contacting the cells with the antibody-drug conjugate of the disclosure, the composition of the disclosure, or the pharmaceutical composition of the disclosure, wherein the cells are cells that express CDH3.

Also provided is a method of preventing or treating a CDH3-mediated disease or condition, comprising administering to a subject in need thereof a prophylactically or therapeutically effective amount of the antibody-drug conjugate of the disclosure, the composition of the disclosure, or the pharmaceutical composition of the disclosure. In some embodiments, the CDH3-mediated disease or condition is a tumor. In some embodiments, the tumor is a CDH3-positive tumor, for example, colorectal cancer, gastric cancer, lung cancer (e.g., non-small cell lung cancer), cervical cancer, pancreatic cancer, esophageal cancer, ovarian cancer, thyroid cancer, bladder cancer, endometrial cancer, breast cancer, liver cancer, prostate cancer, skin cancer, pharyngeal cancer, or bile duct cancer, or any combination thereof.

Also provided is the antibody-drug conjugate of the disclosure, the composition of the disclosure, or the pharmaceutical composition of the disclosure, for preventing or treating a CDH3-mediated disease or condition in a subject, wherein in particular embodiments, the CDH3-mediated disease or condition is a tumor; and in particular embodiments, the tumor is a CDH3-positive tumor, for example, colorectal cancer, gastric cancer, lung cancer (e.g., non-small cell lung cancer), cervical cancer, pancreatic cancer, esophageal cancer, ovarian cancer, thyroid cancer, bladder cancer, endometrial cancer, breast cancer, liver cancer, prostate cancer, skin cancer, pharyngeal cancer, or bile duct cancer, or any combination thereof.

### BRIEF DESCRIPTION OF THE DRAWINGS

FIG. 1A: Results of the detection of anti-human CDH3 antibodies and conjugates binding to OVCAR3 cells.

FIG. 1B: Results of the detection of anti-human CDH3 antibodies and conjugates binding to HCC1569 cells.

FIG. 1C: Results of the detection of anti-human CDH3 antibodies and conjugates binding to NCI-H146 cells.

FIG. 2A: Results of the detection of binding specificity of anti-human CDH3 antibodies and conjugates.

FIG. 2B: Results of the detection of endocytosis specificity of anti-human CDH3 antibodies and conjugates.

FIG. 3: Results of the detection of endocytosis of anti-human CDH3 antibodies and conjugates to NCI-H358 cells.

FIG. 4: Results of the detection of endocytosis of anti-human CDH3 antibodies and conjugates to A431 cells.

FIG. 5: Results of the detection of killing of HCC1954 cells by anti-human CDH3 antibodies and the conjugates of the same toxin.

FIG. 6: Results of the detection of killing of HT29-hCDH3 cells by anti-human CDH3 antibodies and the conjugates of the same toxin.

FIG. 7A: Results of the detection of plasma stability of anti-human CDH3 antibody conjugate Ab-BHZm-A-05.

FIG. 7B: Results of the detection of plasma stability of anti-human CDH3 antibody conjugate Ab-BHZm-A-14.

FIG. 8A: Results of the detection of efficacy of anti-human CDH3 antibody conjugates in the NCI-H358 model.

FIG. 8B: Weight changes in the NCI-H358 model.

FIG. 9A: Results of the detection of efficacy of different anti-human CDH3 antibody conjugates in the NCI-H358 model.

FIG. 9B: Weight changes in the NCI-H358 model.

FIG. 10A: Results of the detection of efficacy of different anti-human CDH3 antibody conjugates in the HCC1954 model.

FIG. 10B: Weight changes in the HCC1954 model.

FIG. 11A: Results of the detection of efficacy of different anti-human CDH3 antibody conjugates in the HCC1806 model.

FIG. 11B: Weight changes in the HCC1806 model.

FIG. 12A: Results of the detection of efficacy of different anti-human CDH3 antibody conjugates in the FADU model.

FIG. 12B: Weight changes in the FADU model.

### DETAILED DESCRIPTION

The present disclosure relates to an antibody-drug conjugate for treating CDH3-positive tumors, and provides an antibody-drug conjugate having a structure shown in a general formula Ab′-(M-L-E-D)ₓ-, with antibodies Ab-BHZ, Ab-AHZ or Ab-BHZm and the like as targeting moieties. The results show that the conjugate has an advantageous drug-antibody conjugation ratio, specific binding to CDH3, and a targeted killing effect on CDH3-positive tumors. Therefore, the present disclosure provides an antibody-drug conjugate for treating CDH3-positive tumors, a pharmaceutical composition comprising the antibody-drug conjugate, and an application thereof in the treatment of CDH3-positive tumors.

### Antibody-Drug Conjugates (ADCs)

In one aspect, the present disclosure provides an antibody-drug conjugate (ADC) represented by formula (I):

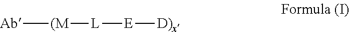

wherein,
Ab′ is an antibody or an antigen-binding fragment thereof;
M is a linker part connecting the antibody or the antigen-binding fragment thereof;
L is a moiety connecting the linker M and E;
E is a moiety connecting L and D;
D is a cytotoxic drug moiety; and
x′ is any integer selected from 1 to 20.

In some embodiments, x′ is any integer selected from 1-16, 1-10, 1-8, 4-8, 4, or 8.

### Antibodies of the ADCs

In some embodiments, the antibody or the antigen-binding fragment thereof is the antibody or the antigen-binding fragment thereof specifically binding to CDH3.

In some embodiments, the antibody or the antigen-binding fragment thereof comprises complementarity determining regions (CDRs) as follows:
(a) CDR-H1, CDR-H2 and CDR-H3 amino acid sequences of a heavy chain variable region (VH) amino acid sequence set forth in SEQ ID NO: 3 or 1; and/or CDR-L1, CDR-L2 and CDR-L3 amino acid sequences of a light chain variable region (VL) amino acid sequence set forth in SEQ ID NO: 4 or 2;
(b) CDR-H1, CDR-H2 and CDR-H3 amino acid sequences of a VH amino acid sequence set forth in SEQ ID NO: 7 or 5; and/or CDR-L1, CDR-L2 and CDR-L3 amino acid sequences of a VL amino acid sequence set forth in SEQ ID NO: 8 or 6;
(c) CDR-H1, CDR-H2 and CDR-H3 amino acid sequences of a VH amino acid sequence set forth in SEQ ID NO: 11 or 9; and/or CDR-L1, CDR-L2 and CDR-L3 amino acid sequences of a VL amino acid sequence set forth in SEQ ID NO: 12 or 10;
(d) CDR-H1, CDR-H2 and CDR-H3 amino acid sequences of a VH amino acid sequence set forth in SEQ ID NO: 15 or 13; and/or CDR-L1, CDR-L2 and CDR-L3 amino acid sequences of a VL amino acid sequence set forth in SEQ ID NO: 14 or 12; or
(e) CDR-H1, CDR-H2 and CDR-H3 amino acid sequences of the following VH, and/or CDR-L1, CDR-L2 and CDR-L3 amino acid sequences of the following VL, wherein at least one CDR of the VH and/or VL comprises a mutation compared to the VH and/or the VL of any one of (a) to (d), and the mutation is a substitution, deletion or addition of one, two or several amino acids (for example, a substitution, deletion or addition of 1, 2, or 3 amino acids); in particular embodiments, the substitution is a conservative substitution.

In some embodiments, the antibody or the antigen-binding fragment thereof comprises complementarity determining regions (CDRs) as follows:
(i) CDR-H1, CDR-H2 and CDR-H3 amino acid sequences of a VH amino acid sequence set forth in SEQ ID NO: 3; and CDR-L1, CDR-L2 and CDR-L3 amino acid sequences of a VL amino acid sequence set forth in SEQ ID NO: 4;
(ii) CDR-H1, CDR-H2 and CDR-H3 amino acid sequences of a VH amino acid sequence set forth in SEQ ID NO: 1; and CDR-L1, CDR-L2 and CDR-L3 amino acid sequences of a VL amino acid sequence set forth in SEQ ID NO: 2;
(iii) CDR-H1, CDR-H2 and CDR-H3 amino acid sequences of a VH amino acid sequence set forth in SEQ ID NO: 7; and CDR-L1, CDR-L2 and CDR-L3 amino acid sequences of a VL amino acid sequence set forth in SEQ ID NO: 8;
(iv) CDR-H1, CDR-H2 and CDR-H3 amino acid sequences of a VH amino acid sequence set forth in SEQ ID NO: 5; and CDR-L1, CDR-L2 and CDR-L3 amino acid sequences of a VL amino acid sequence set forth in SEQ ID NO: 6;
(v) CDR-H1, CDR-H2 and CDR-H3 amino acid sequences of a VH amino acid sequence set forth in SEQ ID NO: 11; and CDR-L1, CDR-L2 and CDR-L3 amino acid sequences of a VL amino acid sequence set forth in SEQ ID NO: 12;
(vi) CDR-H1, CDR-H2 and CDR-H3 amino acid sequences of a VH amino acid sequence set forth in SEQ ID NO: 9; and CDR-L1, CDR-L2 and CDR-L3 amino acid sequences of a VL amino acid sequence set forth in SEQ ID NO: 10;
(vii) CDR-H1, CDR-H2 and CDR-H3 amino acid sequences of a VH amino acid sequence set forth in SEQ ID NO: 15; and CDR-L1, CDR-L2 and CDR-L3 amino acid sequences of a VL amino acid sequence set forth in SEQ ID NO: 16;
(viii) CDR-H1, CDR-H2 and CDR-H3 amino acid sequences of a VH amino acid sequence set forth in SEQ ID NO: 13; and CDR-L1, CDR-L2 and CDR-L3 amino acid sequences of a VL amino acid sequence set forth in SEQ ID NO: 14; or
(ix) CDR-H1, CDR-H2 and CDR-H3 amino acid sequences of the following VH, and/or CDR-L1, CDR-L2 and CDR-L3 amino acid sequences of the following VL, wherein at least one CDR of the VH and/or the VL comprises a mutation compared to the VH and/or the VL of any one of (i) to (viii), and the mutation is a substitution, deletion or addition of one, two or several amino acids (for example, a substitution, deletion or addition of 1, 2, or 3 amino acids); and in particular embodiments, the substitution is a conservative substitution.

In some embodiments, the substitution is a conservative substitution.

In some embodiments, the CDRs are defined according to the IMGT, Kabat, Chothia, Contact or AbM numbering system.

In some embodiments, the antibody or the antigen-binding fragment thereof specifically binding to CDH3 comprises complementarity determining regions (CDRs) as follows:
(a) CDR-H1, CDR-H2 and CDR-H3 amino acid sequences of a VH amino acid sequence set forth in SEQ ID NO: 3; and CDR-L1, CDR-L2 and CDR-L3 amino acid sequences of a VL amino acid sequence set forth in SEQ ID NO: 4;
(b) CDR-H1, CDR-H2 and CDR-H3 amino acid sequences of a VH amino acid sequence set forth in SEQ ID NO: 1; and CDR-L1, CDR-L2 and CDR-L3 amino acid sequences of a VL amino acid sequence set forth in SEQ ID NO: 2;
(c) CDR-H1, CDR-H2 and CDR-H3 amino acid sequences of a VH amino acid sequence set forth in SEQ ID NO: 7; and CDR-L1, CDR-L2 and CDR-L3 amino acid sequences of a VL amino acid sequence set forth in SEQ ID NO: 8;
(d) CDR-H1, CDR-H2 and CDR-H3 amino acid sequences of a VH amino acid sequence set forth in SEQ ID NO: 5; and CDR-L1, CDR-L2 and CDR-L3 amino acid sequences of a VL amino acid sequence set forth in SEQ ID NO: 6;
(e) CDR-H1, CDR-H2 and CDR-H3 amino acid sequences of a VH amino acid sequence set forth in SEQ ID NO: 11; and CDR-L1, CDR-L2 and CDR-L3 amino acid sequences of a VL amino acid sequence set forth in SEQ ID NO: 12;
(f) CDR-H1, CDR-H2 and CDR-H3 amino acid sequences of a VH amino acid sequence set forth in SEQ ID NO: 9; and CDR-L1, CDR-L2 and CDR-L3 amino acid sequences of a VL amino acid sequence set forth in SEQ ID NO: 10;
(g) CDR-H1, CDR-H2 and CDR-H3 amino acid sequences of a VH amino acid sequence set forth in SEQ ID NO: 15; and CDR-L1, CDR-L2 and CDR-L3 amino acid sequences of a VL amino acid sequence set forth in SEQ ID NO: 16; or
(h) CDR-H1, CDR-H2 and CDR-H3 amino acid sequences of a VH amino acid sequence set forth in SEQ ID NO: 13; and CDR-L1, CDR-L2 and CDR-L3 amino acid sequences of a VL amino acid sequence set forth in SEQ ID NO: 14.

In some embodiments, the CDH3 comprises human CDH3 and/or monkey CDH3. In some embodiments, the monkey is Macaca fascicularis.

In some embodiments, the antibody or the antigen-binding fragment thereof comprises a VH and/or a VL, wherein the CDRs are defined according to the Chothia numbering system:
(1a) a VH comprising a CDR-H1 comprising the amino acid sequence of SEQ ID NO: 17 or a variant thereof; a CDR-H2 comprising the amino acid sequence of SEQ ID NO: 27 or a variant thereof; and a CDR-H3 comprising the amino acid sequence of SEQ ID NO: 32 or a variant thereof; and/or a VL comprising a CDR-L1 comprising the amino acid sequence of SEQ ID NO: 35 or a variant thereof; a CDR-L2 comprising the amino acid sequence of SEQ ID NO: 38 or a variant thereof; and a CDR-L3 comprising the amino acid sequence of SEQ ID NO: 42 or a variant thereof;
(1b) a VH comprising a CDR-H1 comprising the amino acid sequence of SEQ ID NO: 17 or a variant thereof; a CDR-H2 comprising the amino acid sequence of SEQ ID NO: 22 or a variant thereof; a CDR-H3 comprising the amino acid sequence of SEQ ID NO: 32 or a variant thereof; and/or a VL comprising a CDR-L1 comprising the amino acid sequence of SEQ ID NO: 35 or a variant thereof; a CDR-L2 comprising the amino acid sequence of SEQ ID NO: 38 or a variant thereof; and a CDR-L3 comprising the amino acid sequence of SEQ ID NO: 42 or a variant thereof;
(1c) a VH comprising a CDR-H1 comprising the amino acid sequence of SEQ ID NO: 44 or a variant thereof; a CDR-H2 comprising the amino acid sequence of SEQ ID NO: 49 or a variant thereof; and a CDR-H3 comprising the amino acid sequence of SEQ ID NO: 55 or a variant thereof; and/or a VL comprising a CDR-L1 comprising the amino acid sequence of SEQ ID NO: 58 or a variant thereof; a CDR-L2 comprising the amino acid sequence of SEQ ID NO: 63 or a variant thereof; and a CDR-L3 comprising the amino acid sequence of SEQ ID NO: 66 or a variant thereof;
(1d) a VH comprising a CDR-H1 comprising the amino acid sequence of SEQ ID NO: 44 or a variant thereof; a CDR-H2 comprising the amino acid sequence of SEQ ID NO: 49 or a variant thereof; and a CDR-H3 comprising the amino acid sequence of SEQ ID NO: 55 or a variant thereof; and/or a VL comprising a CDR-L1 comprising the amino acid sequence of SEQ ID NO: 58 or a variant thereof; a CDR-L2 comprising the amino acid sequence of SEQ ID NO: 61 or a variant thereof; and a CDR-L3 comprising the amino acid sequence of SEQ ID NO: 66 or a variant thereof;
(1e) a VH comprising a CDR-H1 comprising the amino acid sequence of SEQ ID NO: 68 or a variant thereof; a CDR-H2 comprising the amino acid sequence of SEQ ID NO: 73 or a variant thereof; and a CDR-H3 comprising the amino acid sequence of SEQ ID NO: 80 or a variant thereof; and/or a VL comprising a CDR-L1 comprising the amino acid sequence of SEQ ID NO: 84 or a variant thereof; a CDR-L2 comprising the amino acid sequence of SEQ ID NO: 88 or a variant thereof; and a CDR-L3 comprising the amino acid sequence of SEQ ID NO: 91 or a variant thereof;
(1f) a VH comprising a CDR-H1 comprising the amino acid sequence of SEQ ID NO: 68 or a variant thereof; a CDR-H2 comprising the amino acid sequence of SEQ ID NO: 73 or a variant thereof; and a CDR-H3 comprising the amino acid sequence of SEQ ID NO: 80 or a variant thereof; and/or a VL comprising a CDR-L1 comprising the amino acid sequence of SEQ ID NO: 83 or a variant thereof; a CDR-L2 comprising the amino acid sequence of SEQ ID NO: 87 or a variant thereof; and a CDR-L3 comprising the amino acid sequence of SEQ ID NO: 91 or a variant thereof;
(1g) a VH comprising a CDR-H1 comprising the amino acid sequence of SEQ ID NO: 93 or a variant thereof; a CDR-H2 comprising the amino acid sequence of SEQ ID NO: 103 or a variant thereof; and a CDR-H3 comprising the amino acid sequence of SEQ ID NO: 108 or a variant thereof; and/or a VL comprising a CDR-L1 comprising the amino acid sequence of SEQ ID NO: 114 or a variant thereof; a CDR-L2 comprising the amino acid sequence of SEQ ID NO: 117 or a variant thereof; and a CDR-L3 comprising the amino acid sequence of SEQ ID NO: 119 or a variant thereof; or
(1h) a VH comprising a CDR-H1 comprising the amino acid sequence of SEQ ID NO: 93 or a variant thereof; a CDR-H2 comprising the amino acid sequence of SEQ ID NO: 98 or a variant thereof; and a CDR-H3 comprising the amino acid sequence of SEQ ID NO: 108 or a variant thereof; and/or a VL comprising a CDR-L1 comprising the amino acid sequence of SEQ ID NO: 111 or a variant thereof; a CDR-L2 comprising the amino acid sequence of SEQ ID NO: 117 or a variant thereof; and a CDR-L3 comprising the amino acid sequence of SEQ ID NO: 119 or a variant thereof;
wherein the variant in any one of (1a), (1b), (1c), (1d), (1e), (1f), (1g), and (1h) has a substitution, deletion or addition of one, two or several amino acids (for example, a substitution, deletion or addition of 1, 2, or 3 amino acids) compared to the sequence from which it is derived; in particular embodiments, the substitution is a conservative substitution.

In some embodiments, the antibody or the antigen-binding fragment thereof comprises a VH and/or a VL, wherein the CDRs are defined according to the AbM numbering system:
(2a) a VH comprising a CDR-H1 comprising the amino acid sequence of SEQ ID NO: 18 or a variant thereof; a CDR-H2 comprising the amino acid sequence of SEQ ID NO: 28 or a variant thereof; and a CDR-H3 comprising the amino acid sequence of SEQ ID NO: 32 or a variant thereof; and/or a VL comprising a CDR-L1 comprising the amino acid sequence of SEQ ID NO: 35 or a variant thereof; a CDR-L2 comprising the amino acid sequence of SEQ ID NO: 38 or a variant thereof; and a CDR-L3 comprising the amino acid sequence of SEQ ID NO: 42 or a variant thereof;
(2b) a VH comprising a CDR-H1 comprising the amino acid sequence of SEQ ID NO: 18 or a variant thereof; a CDR-H2 comprising the amino acid sequence of SEQ ID NO: 23 or a variant thereof; and a CDR-H3 comprising the amino acid sequence of SEQ ID NO: 32 or a variant thereof; and/or a VL comprising a CDR-L1 comprising the amino acid sequence of SEQ ID NO: 35 or a variant thereof; a CDR-L2 comprising the amino acid sequence of SEQ ID NO: 38 or a variant thereof; and a CDR-L3 comprising the amino acid sequence of SEQ ID NO: 42 or a variant thereof;
(2c) a VH comprising a CDR-H1 comprising the amino acid sequence of SEQ ID NO: 45 or a variant thereof; a CDR-H2 comprising the amino acid sequence of SEQ ID NO: 50 or a variant thereof; and a CDR-H3 comprising the amino acid sequence of SEQ ID NO: 55 or a variant thereof; and/or a VL comprising a CDR-L1 comprising the amino acid sequence of SEQ ID NO: 58 or a variant thereof; a CDR-L2 comprising the amino acid sequence of SEQ ID NO: 63 or a variant thereof; and a CDR-L3 comprising the amino acid sequence of SEQ ID NO: 66 or a variant thereof;
(2d) a VH comprising a CDR-H1 comprising the amino acid sequence of SEQ ID NO: 45 or a variant thereof; a CDR-H2 comprising the amino acid sequence of SEQ ID NO: 50 or a variant thereof; and a CDR-H3 comprising the amino acid sequence of SEQ ID NO: 55 or a variant thereof; and/or a VL comprising a CDR-L1 comprising the amino acid sequence of SEQ ID NO: 58 or a variant thereof; a CDR-L2 comprising the amino acid sequence of SEQ ID NO: 61 or a variant thereof; and a CDR-L3 comprising the amino acid sequence of SEQ ID NO: 66 or a variant thereof;
(2e) a VH comprising a CDR-H1 comprising the amino acid sequence of SEQ ID NO: 69 or a variant thereof; a CDR-H2 comprising the amino acid sequence of SEQ ID NO: 74 or a variant thereof; and a CDR-H3 comprising the amino acid sequence of SEQ ID NO: 80 or a variant thereof; and/or a VL comprising a CDR-L1 comprising the amino acid sequence of SEQ ID NO: 84 or a variant thereof; a CDR-L2 comprising the amino acid sequence of SEQ ID NO: 88 or a variant thereof; and a CDR-L3 comprising the amino acid sequence of SEQ ID NO: 91 or a variant thereof;
(2f) a VH comprising a CDR-H1 comprising the amino acid sequence of SEQ ID NO: 69 or a variant thereof; a CDR-H2 comprising the amino acid sequence of SEQ ID NO: 74 or a variant thereof; and a CDR-H3 comprising the amino acid sequence of SEQ ID NO: 80 or a variant thereof; and/or a VL comprising a CDR-L1 comprising the amino acid sequence of SEQ ID NO: 83 or a variant thereof; a CDR-L2 comprising the amino acid sequence of SEQ ID NO: 87 or a variant thereof; and a CDR-L3 comprising the amino acid sequence of SEQ ID NO: 91 or a variant thereof;
(2g) a VH comprising a CDR-H1 comprising the amino acid sequence of SEQ ID NO: 94 or a variant thereof; a CDR-H2 comprising the amino acid sequence of SEQ ID NO: 104 or a variant thereof; and a CDR-H3 comprising the amino acid sequence of SEQ ID NO: 108 or a variant thereof; and/or a VL comprising a CDR-L1 comprising the amino acid sequence of SEQ ID NO: 114 or a variant thereof; a CDR-L2 comprising the amino acid sequence of SEQ ID NO: 117 or a variant thereof; and a CDR-L3 comprising the amino acid sequence of SEQ ID NO: 119 or a variant thereof; or
(2h) a VH comprising a CDR-H1 comprising the amino acid sequence of SEQ ID NO: 94 or a variant thereof; a CDR-H2 comprising the amino acid sequence of SEQ ID NO: 99 or a variant thereof; and a CDR-H3 comprising the amino acid sequence of SEQ ID NO: 108 or a variant thereof; and/or a VL comprising a CDR-L1 comprising the amino acid sequence of SEQ ID NO: 111 or a variant thereof; a CDR-L2 comprising the amino acid sequence of SEQ ID NO: 117 or a variant thereof; and a CDR-L3 comprising the amino acid sequence of SEQ ID NO: 119 or a variant thereof;
wherein the variant in any one of (2a), (2b), (2c), (2d), (2e), (2f), (2g), and (2h) has a substitution, deletion or addition of one, two or several amino acids (for example, a substitution, deletion or addition of 1, 2, or 3 amino acids) compared to the sequence from which it is derived; in particular embodiments, the substitution is a conservative substitution.

In some embodiments, the antibody or the antigen-binding fragment thereof comprises a VH and/or a VL, wherein the CDRs are defined according to the Kabat numbering system:
(3a) a VH comprising a CDR-H1 comprising the amino acid sequence of SEQ ID NO: 19 or a variant thereof; a CDR-H2 comprising the amino acid sequence of SEQ ID NO: 29 or a variant thereof; and a CDR-H3 comprising the amino acid sequence of SEQ ID NO: 32 or a variant thereof; and/or a VL comprising a CDR-L1 comprising the amino acid sequence of SEQ ID NO: 35 or a variant thereof; a CDR-L2 comprising the amino acid sequence of SEQ ID NO: 38 or a variant thereof; and a CDR-L3 comprising the amino acid sequence of SEQ ID NO: 42 or a variant thereof;
(3b) a VH comprising a CDR-H1 comprising the amino acid sequence of SEQ ID NO: 19 or a variant thereof; a CDR-H2 comprising the amino acid sequence of SEQ ID NO: 24 or a variant thereof; and a CDR-H3 comprising the amino acid sequence of SEQ ID NO: 32 or a variant thereof; and/or a VL comprising a CDR-L1 comprising the amino acid sequence of SEQ ID NO: 35 or a variant thereof; a CDR-L2 comprising the amino acid sequence of SEQ ID NO: 38 or a variant thereof; and a CDR-L3 comprising the amino acid sequence of SEQ ID NO: 42 or a variant thereof;
(3c) a VH comprising a CDR-H1 comprising the amino acid sequence of SEQ ID NO: 46 or a variant thereof; a CDR-H2 comprising the amino acid sequence of SEQ ID NO: 52 or a variant thereof; and a CDR-H3 comprising the amino acid sequence of SEQ ID NO: 55 or a variant thereof; and/or a VL comprising a CDR-L1 comprising the amino acid sequence of SEQ ID NO: 58 or a variant thereof; a CDR-L2 comprising the amino acid sequence of SEQ ID NO: 63 or a variant thereof; and a CDR-L3 comprising the amino acid sequence of SEQ ID NO: 66 or a variant thereof;
(3d) a VH comprising a CDR-H1 comprising the amino acid sequence of SEQ ID NO: 46 or a variant thereof; a CDR-H2 comprising the amino acid sequence of SEQ ID NO: 51 or a variant thereof; and a CDR-H3 comprising the amino acid sequence of SEQ ID NO: 55 or a variant thereof; and/or a VL comprising a CDR-L1 comprising the amino acid sequence of SEQ ID NO: 58 or a variant thereof; a CDR-L2 comprising the amino acid sequence of SEQ ID NO: 61 or a variant thereof; and a CDR-L3 comprising the amino acid sequence of SEQ ID NO: 66 or a variant thereof;
(3e) a VH comprising a CDR-H1 comprising the amino acid sequence of SEQ ID NO: 70 or a variant thereof; a CDR-H2 comprising the amino acid sequence of SEQ ID NO: 77 or a variant thereof; and a CDR-H3 comprising the amino acid sequence of SEQ ID NO: 80 or a variant thereof; and/or a VL comprising a CDR-L1 comprising the amino acid sequence of SEQ ID NO: 84 or a variant thereof; a CDR-L2 comprising the amino acid sequence of SEQ ID NO: 88 or a variant thereof; and a CDR-L3 comprising the amino acid sequence of SEQ ID NO: 91 or a variant thereof;
(3f) a VH comprising a CDR-H1 comprising the amino acid sequence of SEQ ID NO: 70 or a variant thereof; a CDR-H2 comprising the amino acid sequence of SEQ ID NO: 75 or a variant thereof; and a CDR-H3 comprising the amino acid sequence of SEQ ID NO: 80 or a variant thereof; and/or a VL comprising a CDR-L1 comprising the amino acid sequence of SEQ ID NO: 83 or a variant thereof; a CDR-L2 comprising the amino acid sequence of SEQ ID NO: 87 or a variant thereof; and a CDR-L3 comprising the amino acid sequence of SEQ ID NO: 91 or a variant thereof;
(3g) a VH comprising a CDR-H1 comprising the amino acid sequence of SEQ ID NO: 95 or a variant thereof; a CDR-H2 comprising the amino acid sequence of SEQ ID NO: 105 or a variant thereof; and a CDR-H3 comprising the amino acid sequence of SEQ ID NO: 108 or a variant thereof; and/or a VL comprising a CDR-L1 comprising the amino acid sequence of SEQ ID NO: 114 or a variant thereof; a CDR-L2 comprising the amino acid sequence of SEQ ID NO: 117 or a variant thereof; and a CDR-L3 comprising the amino acid sequence of SEQ ID NO: 119 or a variant thereof; or
(3h) a VH comprising a CDR-H1 comprising the amino acid sequence of SEQ ID NO: 95 or a variant thereof; a CDR-H2 comprising the amino acid sequence of SEQ ID NO: 100 or a variant thereof; and a CDR-H3 comprising the amino acid sequence of SEQ ID NO: 108 or a variant thereof; and/or a VL comprising a CDR-L1 comprising the amino acid sequence of SEQ ID NO: 111 or a variant thereof; a CDR-L2 comprising the amino acid sequence of SEQ ID NO: 117 or a variant thereof; and a CDR-L3 comprising the amino acid sequence of SEQ ID NO: 119 or a variant thereof;
wherein the variant in any one of (3a), (3b), (3c), (3d), (3e), (3f), (3g), and (3h) has a substitution, deletion or addition of one, two or several amino acids (for example, a substitution, deletion or addition of 1, 2, or 3 amino acids) compared to the sequence from which it is derived; in particular embodiments, the substitution is a conservative substitution.

In some embodiments, the antibody or the antigen-binding fragment thereof comprises a VH and/or a VL, wherein the CDRs are defined according to the Contact numbering system:
(4a) a VH comprising a CDR-H1 comprising the amino acid sequence of SEQ ID NO: 20 or a variant thereof; a CDR-H2 comprising the amino acid sequence of SEQ ID NO: 30 or a variant thereof; and a CDR-H3 comprising the amino acid sequence of SEQ ID NO: 33 or a variant thereof; and/or a VL comprising a CDR-L1 comprising the amino acid sequence of SEQ ID NO: 36 or a variant thereof; a CDR-L2 comprising the amino acid sequence of SEQ ID NO: 40 or a variant thereof; and a CDR-L3 comprising the amino acid sequence of SEQ ID NO: 43 or a variant thereof;
(4b) a VH comprising a CDR-H1 comprising the amino acid sequence of SEQ ID NO: 20 or a variant thereof; a CDR-H2 comprising the amino acid sequence of SEQ ID NO: 25 or a variant thereof; and a CDR-H3 comprising the amino acid sequence of SEQ ID NO: 33 or a variant thereof; and/or a VL comprising a CDR-L1 comprising the amino acid sequence of SEQ ID NO: 36 or a variant thereof; a CDR-L2 comprising the amino acid sequence of SEQ ID NO: 39 or a variant thereof; and a CDR-L3 comprising the amino acid sequence of SEQ ID NO: 43 or a variant thereof;
(4c) a VH comprising a CDR-H1 comprising the amino acid sequence of SEQ ID NO: 47 or a variant thereof; a CDR-H2 comprising the amino acid sequence of SEQ ID NO: 53 or a variant thereof; and a CDR-H3 comprising the amino acid sequence of SEQ ID NO: 56 or a variant thereof; and/or a VL comprising a CDR-L1 comprising the amino acid sequence of SEQ ID NO: 59 or a variant thereof; a CDR-L2 comprising the amino acid sequence of SEQ ID NO: 64 or a variant thereof; and a CDR-L3 comprising the amino acid sequence of SEQ ID NO: 67 or a variant thereof;
(4d) a VH comprising a CDR-H1 comprising the amino acid sequence of SEQ ID NO: 47 or a variant thereof; a CDR-H2 comprising the amino acid sequence of SEQ ID NO: 53 or a variant thereof; and a CDR-H3 comprising the amino acid sequence of SEQ ID NO: 56 or a variant thereof; and/or a VL comprising a CDR-L1 comprising the amino acid sequence of SEQ ID NO: 59 or a variant thereof; a CDR-L2 comprising the amino acid sequence of SEQ ID NO: 62 or a variant thereof; and a CDR-L3 comprising the amino acid sequence of SEQ ID NO: 67 or a variant thereof;
(4e) a VH comprising a CDR-H1 comprising the amino acid sequence of SEQ ID NO: 71 or a variant thereof; a CDR-H2 comprising the amino acid sequence of SEQ ID NO: 78 or a variant thereof; and a CDR-H3 comprising the amino acid sequence of SEQ ID NO: 81 or a variant thereof; and/or a VL comprising a CDR-L1 comprising the amino acid sequence of SEQ ID NO: 85 or a variant thereof; a CDR-L2 comprising the amino acid sequence of SEQ ID NO: 89 or a variant thereof; and a CDR-L3 comprising the amino acid sequence of SEQ ID NO: 92 or a variant thereof;
(4f) a VH comprising a CDR-H1 comprising the amino acid sequence of SEQ ID NO: 71 or a variant thereof; a CDR-H2 comprising the amino acid sequence of SEQ ID NO: 76 or a variant thereof; and a CDR-H3 comprising the amino acid sequence of SEQ ID NO: 81 or a variant thereof; and/or a VL comprising a CDR-L1 comprising the amino acid sequence of SEQ ID NO: 85 or a variant thereof; a CDR-L2 comprising the amino acid sequence of SEQ ID NO: 89 or a variant thereof; and a CDR-L3 comprising the amino acid sequence of SEQ ID NO: 92 or a variant thereof;
(4g) a VH comprising a CDR-H1 comprising the amino acid sequence of SEQ ID NO: 96 or a variant thereof; a CDR-H2 comprising the amino acid sequence of SEQ ID NO: 106 or a variant thereof; and a CDR-H3 comprising the amino acid sequence of SEQ ID NO: 109 or a variant thereof; and/or a VL comprising a CDR-L1 comprising the amino acid sequence of SEQ ID NO: 115 or a variant thereof; a CDR-L2 comprising the amino acid sequence of SEQ ID NO: 118 or a variant thereof; and a CDR-L3 comprising the amino acid sequence of SEQ ID NO: 120 or a variant thereof; or
(4h) a VH comprising a CDR-H1 comprising the amino acid sequence of SEQ ID NO: 96 or a variant thereof; a CDR-H2 comprising the amino acid sequence of SEQ ID NO: 101 or a variant thereof; and a CDR-H3 comprising the amino acid sequence of SEQ ID NO: 109 or a variant thereof; and/or a VL comprising a CDR-L1 comprising the amino acid sequence of SEQ ID NO: 112 or a variant thereof; a CDR-L2 comprising the amino acid sequence of SEQ ID NO: 118 or a variant thereof; and a CDR-L3 comprising the amino acid sequence of SEQ ID NO: 120 or a variant thereof;
wherein the variant in any one of (4a), (4b), (4c), (4d), (4e), (4f), (4g), and (4h) has a substitution, deletion or addition of one, two or several amino acids (for example, a substitution, deletion or addition of 1, 2, or 3 amino acids) compared to the sequence from which it is derived; in particular embodiments, the substitution is a conservative substitution.

In some embodiments, the antibody or the antigen-binding fragment thereof comprises a VH and/or a VL, wherein the CDRs are defined according to the IMGT numbering system:
(5a) a VH comprising a CDR-H1 comprising the amino acid sequence of SEQ ID NO: 21 or a variant thereof; a CDR-H2 comprising the amino acid sequence of SEQ ID NO: 31 or a variant thereof; and a CDR-H3 comprising the amino acid sequence of SEQ ID NO: 34 or a variant thereof; and/or a VL comprising a CDR-L1 comprising the amino acid sequence of SEQ ID NO: 37 or a variant thereof; a CDR-L2 comprising the amino acid sequence of SEQ ID NO: 41 or a variant thereof; and a CDR-L3 comprising the amino acid sequence of SEQ ID NO: 42 or a variant thereof;
(5b) a VH comprising a CDR-H1 comprising the amino acid sequence of SEQ ID NO: 21 or a variant thereof; a CDR-H2 comprising the amino acid sequence of SEQ ID NO: 26 or a variant thereof; and a CDR-H3 comprising the amino acid sequence of SEQ ID NO: 34 or a variant thereof; and/or a VL comprising a CDR-L1 comprising the amino acid sequence of SEQ ID NO: 37 or a variant thereof; a CDR-L2 comprising the amino acid sequence of SEQ ID NO: 41 or a variant thereof; and a CDR-L3 comprising the amino acid sequence of SEQ ID NO: 42 or a variant thereof;
(5c) a VH comprising a CDR-H1 comprising the amino acid sequence of SEQ ID NO: 48 or a variant thereof; a CDR-H2 comprising the amino acid sequence of SEQ ID NO: 54 or a variant thereof; and a CDR-H3 comprising the amino acid sequence of SEQ ID NO: 57 or a variant thereof; and/or a VL comprising a CDR-L1 comprising the amino acid sequence of SEQ ID NO: 60 or a variant thereof; a CDR-L2 comprising the amino acid sequence of SEQ ID NO: 65 or a variant thereof; and a CDR-L3 comprising the amino acid sequence of SEQ ID NO: 66 or a variant thereof;
(5d) a VH comprising a CDR-H1 comprising the amino acid sequence of SEQ ID NO: 72 or a variant thereof; a CDR-H2 comprising the amino acid sequence of SEQ ID NO: 79 or a variant thereof; and a CDR-H3 comprising the amino acid sequence of SEQ ID NO: 82 or a variant thereof; and/or a VL comprising a CDR-L1 comprising the amino acid sequence of SEQ ID NO: 86 or a variant thereof; a CDR-L2 comprising the amino acid sequence of SEQ ID NO: 90 or a variant thereof; and a CDR-L3 comprising the amino acid sequence of SEQ ID NO: 91 or a variant thereof;
(5e) a VH comprising a CDR-H1 comprising the amino acid sequence of SEQ ID NO: 97 or a variant thereof; a CDR-H2 comprising the amino acid sequence of SEQ ID NO: 107 or a variant thereof; and a CDR-H3 comprising the amino acid sequence of SEQ ID NO: 110 or a variant thereof; and/or a VL comprising a CDR-L1 comprising the amino acid sequence of SEQ ID NO: 116 or a variant thereof; a CDR-L2 comprising the amino acid sequence of SEQ ID NO: 41 or a variant thereof; and a CDR-L3 comprising the amino acid sequence of SEQ ID NO: 119 or a variant thereof; or
(5f) a VH comprising a CDR-H1 comprising the amino acid sequence of SEQ ID NO: 97 or a variant thereof; a CDR-H2 comprising the amino acid sequence of SEQ ID NO: 102 or a variant thereof; and a CDR-H3 comprising the amino acid sequence of SEQ ID NO: 110 or a variant thereof; and/or a VL comprising a CDR-L1 comprising the amino acid sequence of SEQ ID NO: 113 or a variant thereof; a CDR-L2 comprising the amino acid sequence of SEQ ID NO: 41 or a variant thereof; and a CDR-L3 comprising the amino acid sequence of SEQ ID NO: 119 or a variant thereof;
wherein the variant in any one of (5a), (5b), (5c), (5d), (5e), and (5f) has a substitution, deletion or addition of one, two or several amino acids (for example, a substitution, deletion or addition of 1, 2, or 3 amino acids) compared to the sequence from which it is derived; in particular embodiments, the substitution is a conservative substitution.

In some embodiments, the antibody or the antigen-binding fragment thereof comprises the following VH and/or VL, wherein the CDRs are defined according to the Chothia numbering system:
(1a) a VH comprising a CDR-H1 comprising the amino acid sequence of SEQ ID NO: 17; a CDR-H2 comprising the amino acid sequence of SEQ ID NO: 27; a CDR-H3 comprising the amino acid sequence of SEQ ID NO: 32; and/or a VL comprising a CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42;
(1b) a VH comprising CDR-H1 comprising the amino acid sequence of SEQ ID NO: 17; CDR-H2 comprising the amino acid sequence of SEQ ID NO: 22; CDR-H3 comprising the amino acid sequence of SEQ ID NO: 32; and/or a VL comprising CDR-L1 comprising the amino acid sequence of SEQ ID NO: 35; a CDR-L2 comprising the amino acid sequence of SEQ ID NO: 38; and a CDR-L3 comprising the amino acid sequence of SEQ ID NO: 42;
(1c) a VH comprising a CDR-H1 comprising the amino acid sequence of SEQ ID NO: 44; a CDR-H2 comprising the amino acid sequence of SEQ ID NO: 49; a CDR-H3 comprising the amino acid sequence of SEQ ID NO: 55; and/or a VL comprising a CDR-L1 comprising the amino acid sequence of SEQ ID NO: 58; a CDR-L2 comprising the amino acid sequence of SEQ ID NO: 63; and a CDR-L3 comprising the amino acid sequence of SEQ ID NO: 66;
(1d) a VH comprising a CDR-H1 comprising the amino acid sequence of SEQ ID NO: 44; a CDR-H2 comprising the amino acid sequence of SEQ ID NO: 49; a CDR-H3 comprising the amino acid sequence of SEQ ID NO: 55; and/or a VL comprising a CDR-L1 comprising the amino acid sequence of SEQ ID NO: 58; a CDR-L2 comprising the amino acid sequence of SEQ ID NO: 61; and a CDR-L3 comprising the amino acid sequence of SEQ ID NO: 66;
(1e) a VH comprising a CDR-H1 comprising the amino acid sequence of SEQ ID NO: 68; a CDR-H2 comprising the amino acid sequence of SEQ ID NO: 73; a CDR-H3 comprising the amino acid sequence of SEQ ID NO: 80; and/or a VL comprising a CDR-L1 comprising the amino acid sequence of SEQ ID NO: 84; a CDR-L2 comprising the amino acid sequence of SEQ ID NO: 88; and a CDR-L3 comprising the amino acid sequence of SEQ ID NO: 91;
(1f) a VH comprising a CDR-H1 comprising the amino acid sequence of SEQ ID NO: 68; a CDR-H2 comprising the amino acid sequence of SEQ ID NO: 73; a CDR-H3 comprising the amino acid sequence of SEQ ID NO: 80; and/or a VL comprising a CDR-L1 comprising the amino acid sequence of SEQ ID NO: 83; a CDR-L2 comprising the amino acid sequence of SEQ ID NO: 87; and a CDR-L3 comprising the amino acid sequence of SEQ ID NO: 91;
(1g) a VH comprising a CDR-H1 comprising the amino acid sequence of SEQ ID NO: 93; a CDR-H2 comprising the amino acid sequence of SEQ ID NO: 103; a CDR-H3 comprising the amino acid sequence of SEQ ID NO: 108; and/or a VL comprising a CDR-L1 comprising the amino acid sequence of SEQ ID NO: 114; a CDR-L2 comprising the amino acid sequence of SEQ ID NO: 117; and a CDR-L3 comprising the amino acid sequence of SEQ ID NO: 119; or
(1h) a VH comprising a CDR-H1 comprising the amino acid sequence of SEQ ID NO: 93; a CDR-H2 comprising the amino acid sequence of SEQ ID NO: 98; a CDR-H3 comprising the amino acid sequence of SEQ ID NO: 108; and/or a VL comprising a CDR-L1 comprising the amino acid sequence of SEQ ID NO: 111; a CDR-L2 comprising the amino acid sequence of SEQ ID NO: 117; and a CDR-L3 comprising the amino acid sequence of SEQ ID NO: 119.

In some embodiments, the antibody or the antigen-binding fragment thereof comprises the following VH and/or VL, wherein the CDRs are defined according to the AbM numbering system:
(2a) a VH comprising a CDR-H1 comprising the amino acid sequence of SEQ ID NO: 18; a CDR-H2 comprising the amino acid sequence of SEQ ID NO: 28; and a CDR-H3 comprising the amino acid sequence of SEQ ID NO: 32; and/or a VL comprising a CDR-L1 comprising the amino acid sequence of SEQ ID NO: 35; a CDR-L2 comprising the amino acid sequence of SEQ ID NO: 38; and a CDR-L3 comprising the amino acid sequence of SEQ ID NO: 42;
(2b) a VH comprising a CDR-H1 comprising the amino acid sequence of SEQ ID NO: 18; a CDR-H2 comprising the amino acid sequence of SEQ ID NO: 23; and a CDR-H3 comprising the amino acid sequence of SEQ ID NO: 32; and/or a VL comprising a CDR-L1 comprising the amino acid sequence of SEQ ID NO: 35; a CDR-L2 comprising the amino acid sequence of SEQ ID NO: 38; and a CDR-L3 comprising the amino acid sequence of SEQ ID NO: 42;
(2c) a VH comprising a CDR-H1 comprising the amino acid sequence of SEQ ID NO: 45; a CDR-H2 comprising the amino acid sequence of SEQ ID NO: 50; and a CDR-H3 comprising the amino acid sequence of SEQ ID NO: 55; and/or a VL comprising a CDR-L1 comprising the amino acid sequence of SEQ ID NO: 58; a CDR-L2 comprising the amino acid sequence of SEQ ID NO: 63; and a CDR-L3 comprising the amino acid sequence of SEQ ID NO: 66;
(2d) a VH comprising a CDR-H1 comprising the amino acid sequence of SEQ ID NO: 45; a CDR-H2 comprising the amino acid sequence of SEQ ID NO: 50; and a CDR-H3 comprising the amino acid sequence of SEQ ID NO: 55; and/or a VL comprising a CDR-L1 comprising the amino acid sequence of SEQ ID NO: 58; a CDR-L2 comprising the amino acid sequence of SEQ ID NO: 61; and a CDR-L3 comprising the amino acid sequence of SEQ ID NO: 66;
(2e) a VH comprising a CDR-H1 comprising the amino acid sequence of SEQ ID NO: 69; a CDR-H2 comprising the amino acid sequence of SEQ ID NO: 74; and a CDR-H3 comprising the amino acid sequence of SEQ ID NO: 80; and/or a VL comprising a CDR-L1 comprising the amino acid sequence of SEQ ID NO: 84; a CDR-L2 comprising the amino acid sequence of SEQ ID NO: 88; and a CDR-L3 comprising the amino acid sequence of SEQ ID NO: 91;
(2f) a VH comprising a CDR-H1 comprising the amino acid sequence of SEQ ID NO: 69; a CDR-H2 comprising the amino acid sequence of SEQ ID NO: 74; and a CDR-H3 comprising the amino acid sequence of SEQ ID NO: 80; and/or a VL comprising a CDR-L1 comprising the amino acid sequence of SEQ ID NO: 83; a CDR-L2 comprising the amino acid sequence of SEQ ID NO: 87; and a CDR-L3 comprising the amino acid sequence of SEQ ID NO: 91;
(2g) a VH comprising a CDR-H1 comprising the amino acid sequence of SEQ ID NO: 94; a CDR-H2 comprising the amino acid sequence of SEQ ID NO: 104; and a CDR-H3 comprising the amino acid sequence of SEQ ID NO: 108; and/or a VL comprising a CDR-L1 comprising the amino acid sequence of SEQ ID NO: 114; a CDR-L2 comprising the amino acid sequence of SEQ ID NO: 117; and a CDR-L3 comprising the amino acid sequence of SEQ ID NO: 119; or
(2h) a VH comprising a CDR-H1 comprising the amino acid sequence of SEQ ID NO: 94; a CDR-H2 comprising the amino acid sequence of SEQ ID NO: 99; and a CDR-H3 comprising the amino acid sequence of SEQ ID NO: 108; and/or a VL comprising a CDR-L1 comprising the amino acid sequence of SEQ ID NO: 111; CDR-L2 comprising the amino acid sequence of SEQ ID NO: 117; and a CDR-L3 comprising the amino acid sequence of SEQ ID NO: 119.

In some embodiments, the antibody or the antigen-binding fragment thereof comprises the following VH and/or VL, wherein the CDRs are defined according to the Kabat numbering system:
(3a) a VH comprising a CDR-H1 comprising the amino acid sequence of SEQ ID NO: 19; a CDR-H2 comprising the amino acid sequence of SEQ ID NO: 29; and a CDR-H3 comprising the amino acid sequence of SEQ ID NO: 32; and/or a VL comprising a CDR-L1 comprising the amino acid sequence of SEQ ID NO: 35; a CDR-L2 comprising the amino acid sequence of SEQ ID NO: 38; and a CDR-L3 comprising the amino acid sequence of SEQ ID NO: 42;
(3b) a VH comprising a CDR-H1 comprising the amino acid sequence of SEQ ID NO: 19; a CDR-H2 comprising the amino acid sequence of SEQ ID NO: 24; and a CDR-H3 comprising the amino acid sequence of SEQ ID NO: 32; and/or a VL comprising a CDR-L1 comprising the amino acid sequence of SEQ ID NO: 35; a CDR-L2 comprising the amino acid sequence of SEQ ID NO: 38; and a CDR-L3 comprising the amino acid sequence of SEQ ID NO: 42;
(3c) a VH comprising a CDR-H1 comprising the amino acid sequence of SEQ ID NO: 46; a CDR-H2 comprising the amino acid sequence of SEQ ID NO: 52; and a CDR-H3 comprising the amino acid sequence of SEQ ID NO: 55; and/or a VL comprising a CDR-L1 comprising the amino acid sequence of SEQ ID NO: 58; a CDR-L2 comprising the amino acid sequence of SEQ ID NO: 63; and a CDR-L3 comprising the amino acid sequence of SEQ ID NO: 66;
(3d) a VH comprising a CDR-H1 comprising the amino acid sequence of SEQ ID NO: 46; a CDR-H2 comprising the amino acid sequence of SEQ ID NO: 51; and a CDR-H3 comprising the amino acid sequence of SEQ ID NO: 55; and/or a VL comprising a CDR-L1 comprising the amino acid sequence of SEQ ID NO: 58; a CDR-L2 comprising the amino acid sequence of SEQ ID NO: 61; and a CDR-L3 comprising the amino acid sequence of SEQ ID NO: 66;
(3e) a VH comprising a CDR-H1 comprising the amino acid sequence of SEQ ID NO: 70; a CDR-H2 comprising the amino acid sequence of SEQ ID NO: 77; and a CDR-H3 comprising the amino acid sequence of SEQ ID NO: 80; and/or a VL comprising a CDR-L1 comprising the amino acid sequence of SEQ ID NO: 84; a CDR-L2 comprising the amino acid sequence of SEQ ID NO: 88; and a CDR-L3 comprising the amino acid sequence of SEQ ID NO: 91;
(3f) a VH comprising a CDR-H1 comprising the amino acid sequence of SEQ ID NO: 70; a CDR-H2 comprising the amino acid sequence of SEQ ID NO: 75; and a CDR-H3 comprising the amino acid sequence of SEQ ID NO: 80; and/or a VL comprising a CDR-L1 comprising the amino acid sequence of SEQ ID NO: 83; a CDR-L2 comprising the amino acid sequence of SEQ ID NO: 87; and a CDR-L3 comprising the amino acid sequence of SEQ ID NO: 91;
(3g) a VH comprising a CDR-H1 comprising the amino acid sequence of SEQ ID NO: 95; a CDR-H2 comprising the amino acid sequence of SEQ ID NO: 105; and a CDR-H3 comprising the amino acid sequence of SEQ ID NO: 108; and/or a VL comprising a CDR-L1 comprising the amino acid sequence of SEQ ID NO: 114; a CDR-L2 comprising the amino acid sequence of SEQ ID NO: 117; and a CDR-L3 comprising the amino acid sequence of SEQ ID NO: 119; or
(3h) a VH comprising a CDR-H1 comprising the amino acid sequence of SEQ ID NO: 95; a CDR-H2 comprising the amino acid sequence of SEQ ID NO: 100; and a CDR-H3 comprising the amino acid sequence of SEQ ID NO: 108; and/or a VL comprising a CDR-L1 comprising the amino acid sequence of SEQ ID NO: 111; a CDR-L2 comprising the amino acid sequence of SEQ ID NO: 117; and a CDR-L3 comprising the amino acid sequence of SEQ ID NO: 119.

In some embodiments, the antibody or the antigen-binding fragment thereof comprises the following VH and/or VL, wherein the CDRs are defined according to the Contact numbering system:
(4a) a VH comprising a CDR-H1 comprising the amino acid sequence of SEQ ID NO: 20; a CDR-H2 comprising the amino acid sequence of SEQ ID NO: 30; and a CDR-H3 comprising the amino acid sequence of SEQ ID NO: 33; and/or a VL comprising a CDR-L1 comprising the amino acid sequence of SEQ ID NO: 36; a CDR-L2 comprising the amino acid sequence of SEQ ID NO: 40; and a CDR-L3 comprising the amino acid sequence of SEQ ID NO: 43;
(4b) a VH comprising a CDR-H1 comprising the amino acid sequence of SEQ ID NO: 20; a CDR-H2 comprising the amino acid sequence of SEQ ID NO: 25; and a CDR-H3 comprising the amino acid sequence of SEQ ID NO: 33; and/or a VL comprising a CDR-L1 comprising the amino acid sequence of SEQ ID NO: 36; a CDR-L2 comprising the amino acid sequence of SEQ ID NO: 39; and a CDR-L3 comprising the amino acid sequence of SEQ ID NO: 43;
(4c) a VH comprising a CDR-H1 comprising the amino acid sequence of SEQ ID NO: 47; a CDR-H2 comprising the amino acid sequence of SEQ ID NO: 53; and a CDR-H3 comprising the amino acid sequence of SEQ ID NO: 56; and/or a VL comprising a CDR-L1 comprising the amino acid sequence of SEQ ID NO: 59; a CDR-L2 comprising the amino acid sequence of SEQ ID NO: 64; and a CDR-L3 comprising the amino acid sequence of SEQ ID NO: 67;
(4d) a VH comprising a CDR-H1 comprising the amino acid sequence of SEQ ID NO: 47; a CDR-H2 comprising the amino acid sequence of SEQ ID NO: 53; and a CDR-H3 comprising the amino acid sequence of SEQ ID NO: 56; and/or a VL comprising a CDR-L1 comprising the amino acid sequence of SEQ ID NO: 59; a CDR-L2 comprising the amino acid sequence of SEQ ID NO: 62; and a CDR-L3 comprising the amino acid sequence of SEQ ID NO: 67;
(4e) a VH comprising a CDR-H1 comprising the amino acid sequence of SEQ ID NO: 71; a CDR-H2 comprising the amino acid sequence of SEQ ID NO: 78; and a CDR-H3 comprising the amino acid sequence of SEQ ID NO: 81; and/or a VL comprising a CDR-L1 comprising the amino acid sequence of SEQ ID NO: 85; a CDR-L2 comprising the amino acid sequence of SEQ ID NO: 89; and a CDR-L3 comprising the amino acid sequence of SEQ ID NO: 92;
(4f) a VH comprising a CDR-H1 comprising the amino acid sequence of SEQ ID NO: 71; a CDR-H2 comprising the amino acid sequence of SEQ ID NO: 76; and a CDR-H3 comprising the amino acid sequence of SEQ ID NO: 81; and/or a VL comprising a CDR-L1 comprising the amino acid sequence of SEQ ID NO: 85; a CDR-L2 comprising the amino acid sequence of SEQ ID NO: 89; and a CDR-L3 comprising the amino acid sequence of SEQ ID NO: 92;
(4g) a VH comprising a CDR-H1 comprising the amino acid sequence of SEQ ID NO: 96; a CDR-H2 comprising the amino acid sequence of SEQ ID NO: 106; and a CDR-H3 comprising the amino acid sequence of SEQ ID NO: 109; and/or a VL comprising a CDR-L1 comprising the amino acid sequence of SEQ ID NO: 115; a CDR-L2 comprising the amino acid sequence of SEQ ID NO: 118; and a CDR-L3 comprising the amino acid sequence of SEQ ID NO: 120; or
(4h) a VH comprising a CDR-H1 comprising the amino acid sequence of SEQ ID NO: 96; a CDR-H2 comprising the amino acid sequence of SEQ ID NO: 101; and a CDR-H3 comprising the amino acid sequence of SEQ ID NO: 109; and/or a VL comprising a CDR-L1 comprising the amino acid sequence of SEQ ID NO: 112; a CDR-L2 comprising the amino acid sequence of SEQ ID NO: 118; and a CDR-L3 comprising the amino acid sequence of SEQ ID NO: 120.

In some embodiments, the antibody or the antigen-binding fragment thereof comprises the following VH and/or VL, wherein the CDRs are defined according to the IMGT numbering system:
(5a) a VH comprising a CDR-H1 comprising the amino acid sequence of SEQ ID NO: 21; a CDR-H2 comprising the amino acid sequence of SEQ ID NO: 31; and a CDR-H3 comprising the amino acid sequence of SEQ ID NO: 34; and/or a VL comprising a CDR-L1 comprising the amino acid sequence of SEQ ID NO: 37; a CDR-L2 comprising the amino acid sequence of SEQ ID NO: 41; and a CDR-L3 comprising the amino acid sequence of SEQ ID NO: 42;
(5b) a VH comprising a CDR-H1 comprising the amino acid sequence of SEQ ID NO: 21; a CDR-H2 comprising the amino acid sequence of SEQ ID NO: 26; and a CDR-H3 comprising the amino acid sequence of SEQ ID NO: 34; and/or a VL comprising a CDR-L1 comprising the amino acid sequence of SEQ ID NO: 37; a CDR-L2 comprising the amino acid sequence of SEQ ID NO: 41; and a CDR-L3 comprising the amino acid sequence of SEQ ID NO: 42;
(5c) a VH comprising a CDR-H1 comprising the amino acid sequence of SEQ ID NO: 48; a CDR-H2 comprising the amino acid sequence of SEQ ID NO: 54; and a CDR-H3 comprising the amino acid sequence of SEQ ID NO: 57; and/or a VL comprising a CDR-L1 comprising the amino acid sequence of SEQ ID NO: 60; a CDR-L2 comprising the amino acid sequence of SEQ ID NO: 65; and a CDR-L3 comprising the amino acid sequence of SEQ ID NO: 66;
(5d) a VH comprising a CDR-H1 comprising the amino acid sequence of SEQ ID NO: 72; a CDR-H2 comprising the amino acid sequence of SEQ ID NO: 79; and a CDR-H3 comprising the amino acid sequence of SEQ ID NO: 82; and/or a VL comprising a CDR-L1 comprising the amino acid sequence of SEQ ID NO: 86; a CDR-L2 comprising the amino acid sequence of SEQ ID NO: 90; and a CDR-L3 comprising the amino acid sequence of SEQ ID NO: 91;
(5e) a VH comprising a CDR-H1 comprising the amino acid sequence of SEQ ID NO: 97; a CDR-H2 comprising the amino acid sequence of SEQ ID NO: 107; and a CDR-H3 comprising the amino acid sequence of SEQ ID NO: 110; and/or a VL comprising a CDR-L1 comprising the amino acid sequence of SEQ ID NO: 116; a CDR-L2 comprising the amino acid sequence of SEQ ID NO: 41; and a CDR-L3 comprising the amino acid sequence of SEQ ID NO: 119; or
(5f) a VH comprising a CDR-H1 comprising the amino acid sequence of SEQ ID NO: 97; a CDR-H2 comprising the amino acid sequence of SEQ ID NO: 102; and a CDR-H3 comprising the amino acid sequence of SEQ ID NO: 110; and/or a VL comprising a CDR-L1 comprising the amino acid sequence of SEQ ID NO: 113; a CDR-L2 comprising the amino acid sequence of SEQ ID NO: 41; and a CDR-L3 comprising the amino acid sequence of SEQ ID NO: 119.

In some embodiments, the antibody or the antigen binding fragment thereof comprises a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 1, 3, 5, 7, 9, 11, 13 or 15. In some embodiments, the antibody or the antigen binding fragment thereof comprises a VH comprising an amino acid sequence set forth in SEQ ID NO: 1, 3, 5, 7, 9, 11, 13 or 15. In some embodiments, the antibody or the antigen binding fragment thereof comprises a VH consisting of an amino acid sequence set forth in SEQ ID NO: 1, 3, 5, 7, 9, 11, 13 or 15.

In some embodiments, the antibody or the antigen binding fragment thereof comprises a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 2, 4, 6, 8, 10, 12, 14 or 16. In some embodiments, the antibody or the antigen binding fragment thereof comprises a VL comprising an amino acid sequence set forth in SEQ ID NO: 2, 4, 6, 8, 10, 12, 14 or 16. In some embodiments, the antibody or the antigen binding fragment thereof comprises a VL consisting of an amino acid sequence set forth in SEQ ID NO: 2, 4, 6, 8, 10, 12, 14 or 16.

In some embodiments, the antibody or the antigen-binding fragment thereof comprises:
(a) a VH comprising the amino acid sequence set forth in SEQ ID NO: 3 or a variant thereof and/or a VL comprising the amino acid sequence set forth in SEQ ID NO: 4 or a variant thereof;
(b) a VH comprising the amino acid sequence set forth in SEQ ID NO: 1 or a variant thereof and/or a VL comprising the amino acid sequence set forth in SEQ ID NO: 2 or a variant thereof;
(c) a VH comprising the amino acid sequence set forth in SEQ ID NO: 7 or a variant thereof and/or a VL comprising the amino acid sequence set forth in SEQ ID NO: 8 or a variant thereof;
(d) a VH comprising the amino acid sequence set forth in SEQ ID NO: 5 or a variant thereof and/or a VL comprising the amino acid sequence set forth in SEQ ID NO: 6 or a variant thereof;
(e) a VH comprising the amino acid sequence set forth in SEQ ID NO: 11 or a variant thereof and/or a VL comprising the amino acid sequence set forth in SEQ ID NO: 12 or a variant thereof;
(f) a VH comprising the amino acid sequence set forth in SEQ ID NO: 9 or a variant thereof and/or a VL comprising the amino acid sequence set forth in SEQ ID NO: 10 or a variant thereof;
(g) a VH comprising the amino acid sequence set forth in SEQ ID NO: 15 or a variant thereof and/or a VL comprising the amino acid sequence set forth in SEQ ID NO: 16 or a variant thereof; or
(h) a VH comprising the amino acid sequence set forth in SEQ ID NO: 13 or a variant thereof and/or a VL comprising the amino acid sequence set forth in SEQ ID NO: 14 or a variant thereof;
wherein the variant has at least 80%, at least 85%, at least 90%, at least 91%, at least 92%, at least 93%, at least 94%, at least 95%, at least 96%, at least 97%, at least 98%, at least 99% or 100% sequence identity compared to the sequence from which it is derived, or has a substitution, deletion or addition of one, two or several amino acids (for example, a substitution, deletion or addition of 1, 2, 3, 4, or 5 amino acids) compared to the sequence from which it is derived. In particular embodiments, the substitution is a conservative substitution.

In some embodiments, the antibody or the antigen-binding fragment thereof comprises:
(a) a VH comprising the amino acid sequence set forth in SEQ ID NO: 3 and a VL comprising the amino acid sequence set forth in SEQ ID NO: 4;
(b) a VH comprising the amino acid sequence set forth in SEQ ID NO: 1 and a VL comprising the amino acid sequence set forth in SEQ ID NO: 2;
(c) a VH comprising the amino acid sequence set forth in SEQ ID NO: 7 and a VL comprising the amino acid sequence set forth in SEQ ID NO: 8;
(d) a VH comprising the amino acid sequence set forth in SEQ ID NO: 5 and a VL comprising the amino acid sequence set forth in SEQ ID NO: 6;
(e) a VH comprising the amino acid sequence set forth in SEQ ID NO: 11 and a VL comprising the amino acid sequence set forth in SEQ ID NO: 12;
(f) a VH comprising the amino acid sequence set forth in SEQ ID NO: 9 and a VL comprising the amino acid sequence set forth in SEQ ID NO: 10;
(g) a VH comprising the amino acid sequence set forth in SEQ ID NO: 15 and a VL comprising the amino acid sequence set forth in SEQ ID NO: 16; or
(h) a VH comprising the amino acid sequence set forth in SEQ ID NO: 13 and a VL comprising the amino acid sequence set forth in SEQ ID NO: 14.

In some embodiments, the antibody or the antigen-binding fragment disclosed herein comprises an antibody or an antigen-binding fragment specifically binding to an antigen, and may comprise a post-translational modification thereof (for example, cleavage of C-terminal lysine in a heavy chain, and conversion of N-terminal glutamine or glutamic acid in a heavy chain or a light chain into pyroglutamic acid or pyroglutamate), which may occur at the time of recombinant expression in a host cell (for example, a CHO cell) or during purification/storage.

In some embodiments of the antibody or the antigen-binding fragment disclosed herein, a heavy chain constant domain may comprise C-terminal lysine or lack C-terminal lysine or C-terminal glycine-lysine dipeptide. In some embodiments of an antibody or an antigen-binding fragment thereof, an N-terminal amino acid of the antibody or the antigen-binding fragment thereof may be cyclized to pyroglutamic acid. In some embodiments of an antibody or an antigen-binding fragment thereof, an N-terminal amino acid of the antibody or the antigen-binding fragment thereof may be cyclized to pyroglutamate.

As known to those skilled in the art, the pyroglutamic acid is a conjugate acid of pyroglutamate and is in mutual equilibrium with pyroglutamate in a solution.

In some embodiments, provided herein is a composition comprising the antibody or the antigen-binding fragment disclosed herein, wherein various antibodies or antigen-binding fragments can independently comprise C-terminal lysine, lack C-terminal lysine, lack C-terminal glycine-lysine, and/or comprise N-terminal glutamine or glutamic acid, cyclization of an N-terminal amino acid to pyroglutamic acid or cyclization of the N-terminal amino acid to pyroglutamate.

In some embodiments, an N-terminal glutamine of the VH comprising the amino acid sequence set forth in any one of SEQ ID NO: 3, 1, 7, 5, 11, 9, 15, and 13 or a variant thereof is subjected to cyclization to form pyroglutamic acid or pyroglutamate.

In some embodiments, the antibody or the antigen-binding fragment thereof of any embodiment above further has a feature selected from the following:
(1) specifically binding to human or monkey CDH3, for example, determined by ELISA or flow cytometry;
(2) not binding or substantially not binding to CDH1 and CDH2, for example, determined by flow cytometry;
(3) having a reduced or eliminated ADCC activity and/or ADCP activity and/or CDC activity;
(4) having a reduced CDC activity;
(5) inducing CDH3 internalization, for example, determined by flow cytometry;
(6) inhibiting cell (such as, tumor cell) proliferation; and/or
(7) inhibiting tumor growth.

In some embodiments, the antibody or the antigen-binding fragment thereof of any embodiment above may comprise a constant region from or derived from human immunoglobulin.

In some embodiments, a heavy chain of the antibody or the antigen-binding fragment thereof comprises a heavy chain constant region from or derived from human immunoglobulin (for example, IgG1, IgG2, IgG3, or IgG4). In some embodiments, a heavy chain of the antibody or the antigen-binding fragment thereof comprises a wild-type Fc region, or comprises a mutated or chemically modified Fc region, which has an altered effector function (for example, a reduced ADCC activity, ADCP activity, and/or CDC activity) compared to the wild-type Fc region. In certain exemplary embodiments, the antibody or the antigen-binding fragment thereof comprises a variant of a human IgG1 heavy chain constant region, and the variant has the following substitutions compared to the wild-type sequence from which it is derived: Leu234Ala, Leu235Ala, and Gly237Ala (positions according to the EU numbering system). In such embodiments, the antibody or the antigen-binding fragment thereof has a reduced ADCC activity, ADCP activity, and/or CDC activity. In some embodiments, the antibody or the antigen-binding fragment thereof comprises a variant of a human IgG1 heavy chain constant region comprising the amino acid sequence set forth in SEQ ID NO: 122. In some embodiments, the heavy chain constant region (CH) comprising the amino acid sequence set forth in SEQ ID NO: 122 or a variant thereof lacks C-terminal lysine. In some embodiments, a heavy chain of the antibody or the antigen-binding fragment thereof comprises the amino acid sequence set forth in SEQ ID NO: 121 or a variant thereof, and the variant has a conservative substitution of at most 20 amino acids (for example, a conservative substitution of at most 15, at most 10, or at most 5 amino acids; for example, a conservative substitution of 1, 2, 3, 4, or 5 amino acids) compared thereto. In some embodiments, the CH comprising the amino acid sequence set forth in SEQ ID NO: 121 or a variant thereof lacks a C-terminal lysine.

In some embodiments, a light chain of the antibody or the antigen-binding fragment thereof comprises a light chain constant region (CL) from or derived from human immunoglobulin (for example, κ or λ). In some embodiments, a light chain of the antibody or the antigen-binding fragment thereof comprises the amino acid sequence set forth in SEQ ID NO: 123 or a variant thereof, and the variant has a conservative substitution of at most 20 amino acids (for example, a conservative substitution of at most 15, at most 10, or at most 5 amino acids; for example, a conservative substitution of 1, 2, 3, 4, or 5 amino acids) compared thereto. In some embodiments, a light chain of the antibody or the antigen-binding fragment thereof comprises the amino acid sequence set forth in SEQ ID NO: 144 or a variant thereof, and the variant has a conservative substitution of at most 20 amino acids (for example, a conservative substitution of at most 15, at most 10, or at most 5 amino acids; for example, a conservative substitution of 1, 2, 3, 4, or 5 amino acids) compared thereto.

In some embodiments, the antibody or the antigen-binding fragment thereof comprises:
(1) a heavy chain comprising a VH amino acid sequence set forth in SEQ ID NO: 3 and a CH amino acid sequence set forth in SEQ ID NO: 122, and a light chain comprising a VL amino acid sequence set forth in SEQ ID NO: 4 and a CL amino acid sequence set forth in SEQ ID NO: 123;
(2) a heavy chain comprising a VH amino acid sequence set forth in SEQ ID NO: 3 and a CH amino acid sequence set forth in SEQ ID NO: 121, and a light chain comprising a VL amino acid sequence set forth in SEQ ID NO: 4 and a CL amino acid sequence set forth in SEQ ID NO: 123;
(3) a heavy chain comprising a VH amino acid sequence set forth in SEQ ID NO: 1 and a CH amino acid sequence set forth in SEQ ID NO: 121, and a light chain comprising a VL amino acid sequence set forth in SEQ ID NO: 2 and a CL amino acid sequence set forth in SEQ ID NO: 123;
(4) a heavy chain comprising a VH amino acid sequence set forth in SEQ ID NO: 7 and a CH amino acid sequence set forth in SEQ ID NO: 121, and a light chain comprising a VL amino acid sequence set forth in SEQ ID NO: 8 and a CL amino acid sequence set forth in SEQ ID NO: 144;
(5) a heavy chain comprising a VH amino acid sequence set forth in SEQ ID NO: 5 and a CH amino acid sequence set forth in SEQ ID NO: 121, and a light chain comprising a VL amino acid sequence set forth in SEQ ID NO: 6 and a CL amino acid sequence set forth in SEQ ID NO: 144;
(6) a heavy chain comprising a VH amino acid sequence set forth in SEQ ID NO: 11 and a CH amino acid sequence set forth in SEQ ID NO: 121, and a light chain comprising a VL amino acid sequence set forth in SEQ ID NO: 12 and a CL amino acid sequence set forth in SEQ ID NO: 123;
(7) a heavy chain comprising a VH amino acid sequence set forth in SEQ ID NO: 9 and a CH amino acid sequence set forth in SEQ ID NO: 121, and a light chain comprising a VL amino acid sequence set forth in SEQ ID NO: 10 and a CL amino acid sequence set forth in SEQ ID NO: 123;
(8) a heavy chain comprising a VH amino acid sequence set forth in SEQ ID NO: 15 and a CH amino acid sequence set forth in SEQ ID NO: 121, and a light chain comprising a VL amino acid sequence set forth in SEQ ID NO: 16 and a CL amino acid sequence set forth in SEQ ID NO:123; or
(9) a heavy chain comprising a VH amino acid sequence set forth in SEQ ID NO: 13 and a CH amino acid sequence set forth in SEQ ID NO: 121, and a light chain comprising a VL amino acid sequence set forth in SEQ ID NO: 14 and a CL amino acid sequence set forth in SEQ ID NO: 123.

In some embodiments, the antibody or the antigen-binding fragment thereof comprises:
(1) a heavy chain comprising the amino acid sequence set forth in SEQ ID NO: 126, and a light chain comprising the amino acid sequence set forth in SEQ ID NO: 125;
(2) a heavy chain comprising the amino acid sequence set forth in SEQ ID NO: 124, and a light chain comprising the amino acid sequence set forth in SEQ ID NO: 125;
(3) a heavy chain comprising the amino acid sequence set forth in SEQ ID NO: 127, and a light chain comprising the amino acid sequence set forth in SEQ ID NO: 128;
(4) a heavy chain comprising the amino acid sequence set forth in SEQ ID NO: 129, and a light chain comprising the amino acid sequence set forth in SEQ ID NO: 130;
(5) a heavy chain comprising the amino acid sequence set forth in SEQ ID NO: 131, and a light chain comprising the amino acid sequence set forth in SEQ ID NO: 132;
(6) a heavy chain comprising the amino acid sequence set forth in SEQ ID NO: 133, and a light chain comprising the amino acid sequence set forth in SEQ ID NO: 134;
(7) a heavy chain comprising the amino acid sequence set forth in SEQ ID NO: 135, and a light chain comprising the amino acid sequence set forth in SEQ ID NO: 136;
(8) a heavy chain comprising the amino acid sequence set forth in SEQ ID NO: 137, and a light chain comprising the amino acid sequence set forth in SEQ ID NO: 138; or
(9) a heavy chain comprising the amino acid sequence set forth in SEQ ID NO: 139, and a light chain comprising the amino acid sequence set forth in SEQ ID NO: 140.

In some embodiments, N-terminal glutamine of the heavy chain comprising the amino acid sequence set forth in any one of SEQ ID NOs: 126, 124, 127, 129, 131, 133, 135, 137, and 139 is subjected to cyclization to form pyroglutamic acid or pyroglutamate.

In some embodiments, the heavy chain comprising the amino acid sequence set forth in any one of SEQ ID NOs: 126, 124, 127, 129, 131, 133, 135, 137, and 139 lacks C-terminal lysine.

In some embodiments, N-terminal glutamine of the heavy chain comprising the amino acid sequence set forth in any one of SEQ ID NOs: 126, 124, 127, 129, 131, 133, 135, 137, and 139 is subjected to cyclization to form pyroglutamic acid or pyroglutamate, and lacks C-terminal lysine.

In some embodiments, the antibody of the present disclosure comprises: a heavy chain comprising the amino acid sequence set forth in SEQ ID NO: 126, and a light chain comprising the amino acid sequence set forth in SEQ ID NO: 125.

In some embodiments, the antibody of the present disclosure comprises: a heavy chain comprising the amino acid sequence set forth in SEQ ID NO: 141 and a light chain comprising the amino acid sequence set forth in SEQ ID NO: 125.

In some embodiments, the antibody of the present disclosure comprises: a heavy chain comprising the amino acid sequence set forth in SEQ ID NO: 142 and a light chain comprising the amino acid sequence set forth in SEQ ID NO: 125.

In some embodiments, the antibody of the present disclosure comprises: a heavy chain comprising the amino acid sequence set forth in SEQ ID NO: 143 and a light chain comprising the amino acid sequence set forth in SEQ ID NO: 125.

In some embodiments, the antibody or the antigen-binding fragment thereof of any embodiment above is a murine antibody, a chimeric antibody, or a humanized antibody.

In some embodiments, a variable region of the antibody or the antigen-binding fragment thereof of any embodiment above is human-derived.

In some embodiments, the antibody or the antigen-binding fragment thereof of any embodiment above is selected from ScFv, Fab, Fab′, F(ab′)₂, Fab′-SH, Fv fragments, disulfide-linked Fv (dsFv), a diabody, a bispecific antibody and a multispecific antibody.

An antibody of the present disclosure can be prepared by various methods known in the art, for example, obtained by a genetic engineering recombinant technology. For example, a DNA molecule encoding the heavy chain and light chain genes of the antibody of the present disclosure is obtained by chemical synthesis or PCR amplification. The resulting DNA molecule is inserted into an expression vector and then transfected into a host cell. Then, the transfected host cell is cultured under specific conditions and the antibody of the present disclosure is expressed.

The antigen-binding fragment of the present disclosure can be obtained by hydrolysis of an intact antibody molecule (see Morimoto et al., J. Biochem. Biophys. Methods 24:107-117 (1992) and Brennan et al., Science 229:81 (1985)). In addition, these antigen-binding fragments can also be directly produced by a recombinant host cell (reviewed in Hudson, Curr. Opin. Immunol. 11: 548-557 (1999); Little et al., Immunol. Today, 21: 364-370 (2000)). For example, a Fab′ fragment can be directly obtained from a host cell; Fab′ fragments can be chemically conjugated to form an F(ab′)₂ segment (Carter et al., Bio/Technology 10: 163-167 (1992)). In addition, Fv, Fab or F(ab′)₂ fragments can also be obtained by directly isolating from a recombinant host cell culture medium. Other technologies for preparing these antigen-binding fragments are fully known to those skilled in the art.

### Drug-Linkers of the ADCs

The ADCs of the disclosure provided herein comprise an antibody as described herein and further comprise a cytotoxic drug moiety D, which is linked to the antibody via a linker -M-L-E-.

In some embodiments, formula (I) is further Ab-(Y-M-L-E-D)ₓ, wherein Ab-(Y-represents an antibody or an antigen-binding fragment thereof as described above, and —(Y-represents an attachment moiety between an amino acid residue in the antibody or the antigen-binding fragment thereof and M.

In some embodiments, the amino acid residue is a cysteine, lysine, serine or threonine residue.

In some embodiments, Y is S, NH or 0.

In the antibody-drug conjugate, the cytotoxic drug can be attached to the antibody or the antigen-binding fragment thereof by a linker (such as the “M-L-E” fragment shown in the present disclosure).

In some embodiments, M is

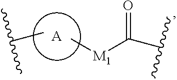

wherein ring A is a 5-6-membered aliphatic heterocyclic ring, or a 5-20-membered aromatic ring system, and the aliphatic heterocyclic ring and the aromatic ring system are optionally substituted with one or more groups selected from oxo (=O), halogen, cyano, amino, carboxyl, sulfhydryl, and C₁₋₆alkyl; and M₁ is selected from a single bond, C₁₋₂₀alkylene, C₂₋₂₀alkenylene, and C₂₋₂₀alkynylene.

In some embodiments, M is

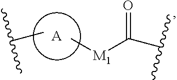

wherein ring A is a 5-membered aliphatic heterocylic ring, a 6-membered heteroaromatic ring, or a polycyclic ring formed by connecting more than one 6-membered heteroaromatic ring with a phenyl ring via a single bond, and the aliphatic heterocyclic ring is optionally substituted with one or more groups selected from oxo (=O), halogen, and C₁₋₄alkyl; and M₁ is selected from a single bond, C₃₋₁₀alkylene, C₃₋₁₀alkenylene, and C₃₋₁₀alkynylene.

In some embodiments, M is

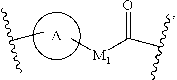

wherein ring A is selected from

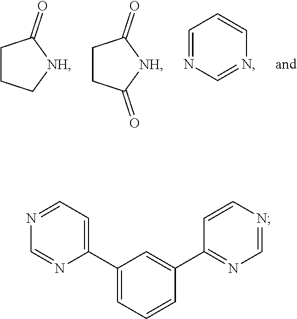

M₁ is selected from a single bond, C₅₋₈alkylene, C₅₋₈alkenylene, and C₅₋₈alkynylene.

In some embodiments, M is selected from the following structures:

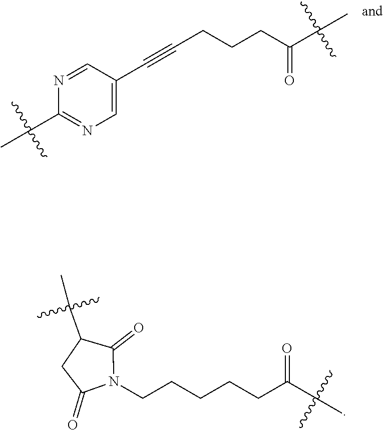

In some embodiments, M is selected from the following structures:

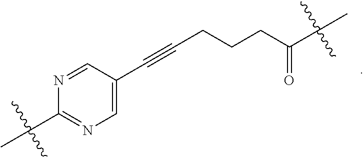 [SMILES: *C(=O)CCCC#Cc1cnc(*)nc1]

In some embodiments, L is selected from a structure consisting of one or more of the following: C₁₋₆alkylene, —N(R′)—, carbonyl, —O—, natural amino acids or unnatural amino acids and their analogs (for example, Ala, Arg, Asn, Asp, Cit, Cys, Gln, Glu, Gly, His, Ile, Leu, Lys, Met, Phe, Pro, Ser, Thr, Trp, Tyr, Val, Lys(COCH₂CH₂(OCH₂CH₂)ᵣOCH₃)), and short peptides consisting of amino acids (for example, Ala-Ala, Ala-Lys, Ala-Lys(Ac), Ala-Pro, Gly-Glu, Gly-Gly, Phe-Lys, Phe-Lys(Ac), Val-Ala, Val-Lys, Val-Lys(Ac), Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly (GGFG, SEQ ID NO: 145), Gly-Gly-Val-Ala (GGVA, SEQ ID NO: 146), Gly-Phe-Leu-Gly (GFLG, SEQ ID NO: 147), Glu-Ala-Ala-Ala (EAAA, SEQ ID NO: 148), and Gly-Gly-Gly-Gly-Gly (GGGGG, SEQ ID NO: 149)),

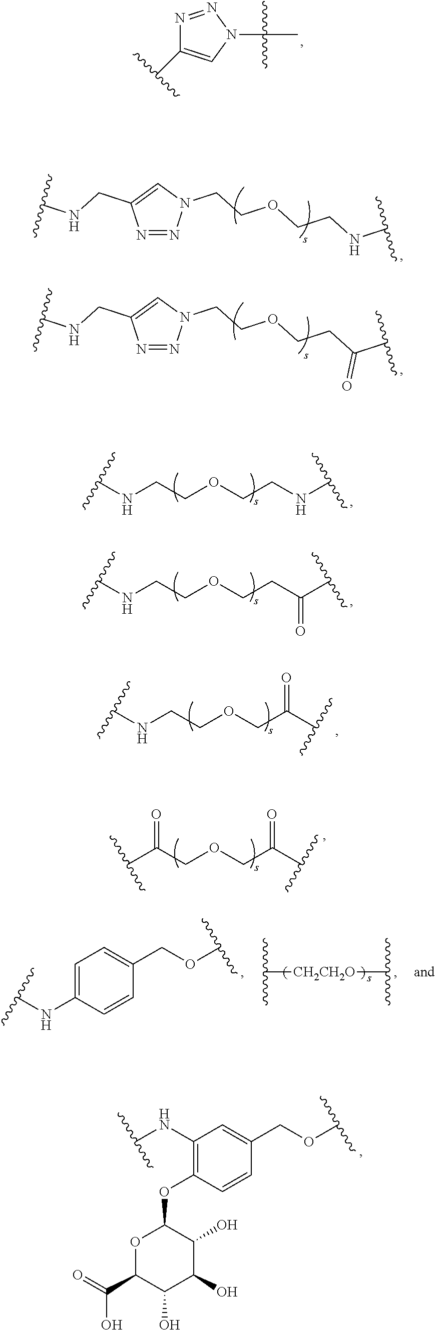

or a combination thereof; wherein R′ represents hydrogen, C₁₋₆alkyl, or a polyethylene glycol fragment comprising 1-10 ethylene oxide units; s is selected from integers of 1-20, for example, integers of 1-15, 1-10, 1-8, 1-6, 1-4, 1-2 and 3-6, for example, s is 1, 2, 3, 4, 5, 6, 7, 8, 9, 10, 11, 12, 13, 14, or 15.

In some embodiments, L is selected from the structures consisting of one or more of the following: C₁₋₆alkylene, carbonyl, —NH—, Ala-Ala, Ala-Lys, Ala-Pro, Gly-Glu, Gly-Gly, Phe-Lys, Val-Ala, Val-Lys, Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly,

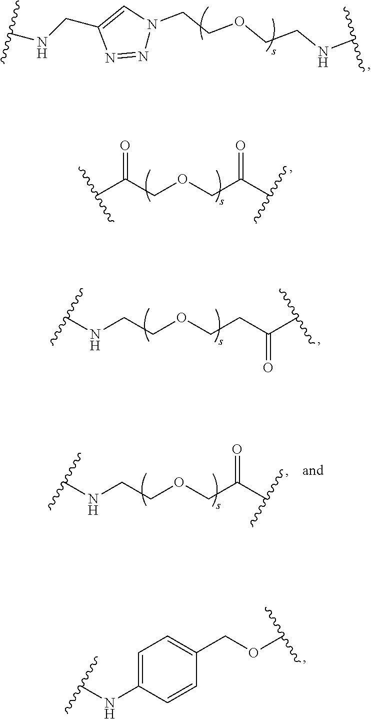

or a combination thereof; wherein s is selected from integers of 1-20, for example, integers of 1-15, 1-10, 1-8, 1-6, 1-4, 1-2 and 3-6, for example, s is 1, 2, 3, 4, 5, 6, 7, 8, 9, 10, 11, 12, 13, 14, or 15.

In some embodiments, L is selected from the following structures:

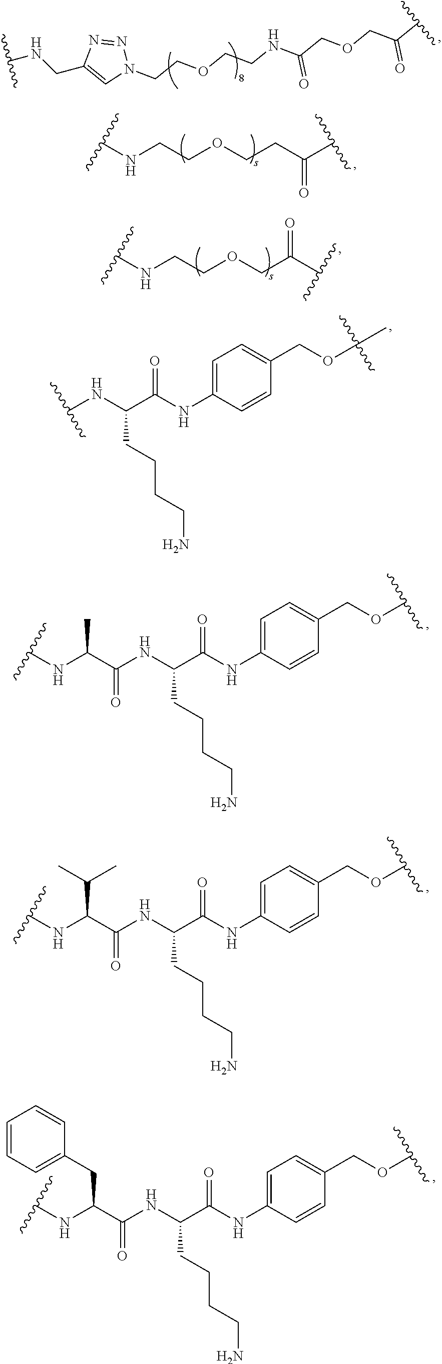

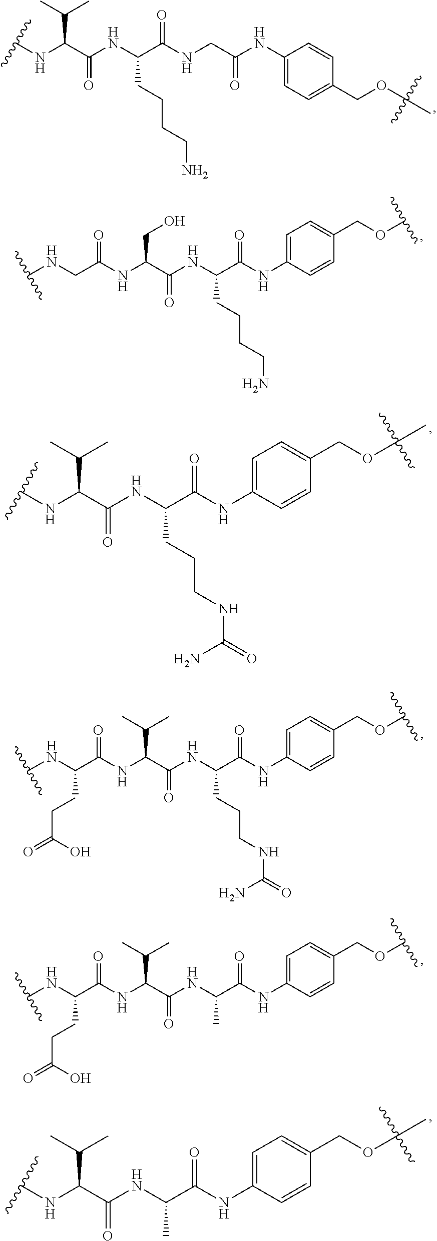 [SMILES: *N[C@H](C(=O)N[C@@H](CCCCN)C(=O)NCC(=O)Nc1ccc(CO*)cc1)C(C)C ; *NCC(=O)N[C@@H](CO)C(=O)N[C@@H](CCCCN)C(=O)Nc1ccc(CO*)cc1 ; *N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](C)C(=O)Nc1ccc(CO*)cc1)C(C)C ; *N[C@H](C(=O)N[C@@H](C)C(=O)Nc1ccc(CO*)cc1)C(C)C]

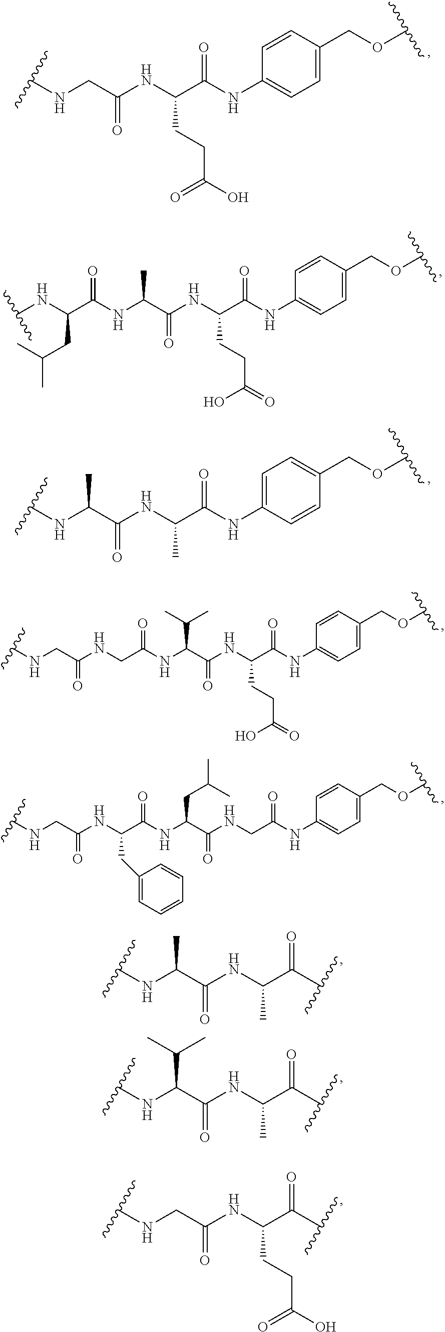

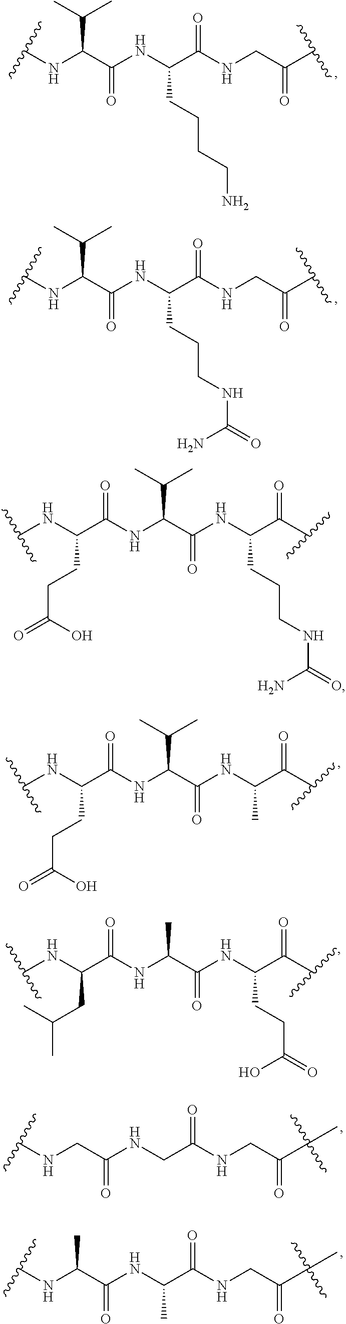

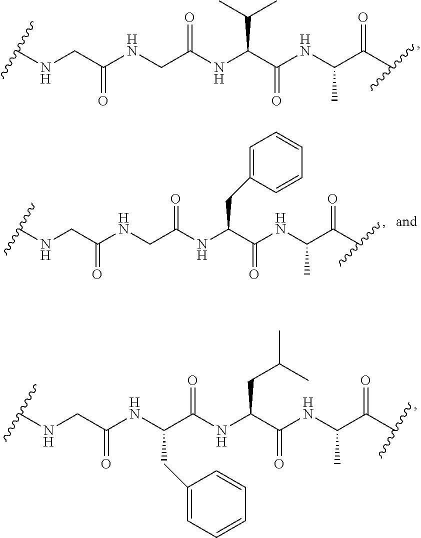

In some embodiments, L is selected from the following structures:

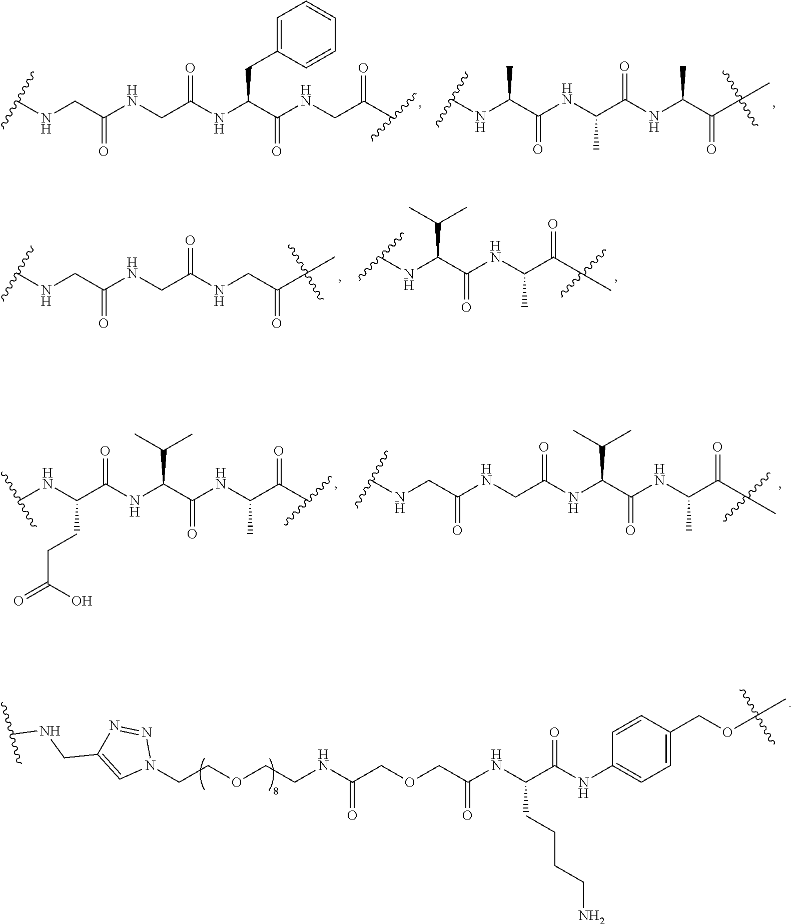

In some embodiments, L is selected from the following structures:

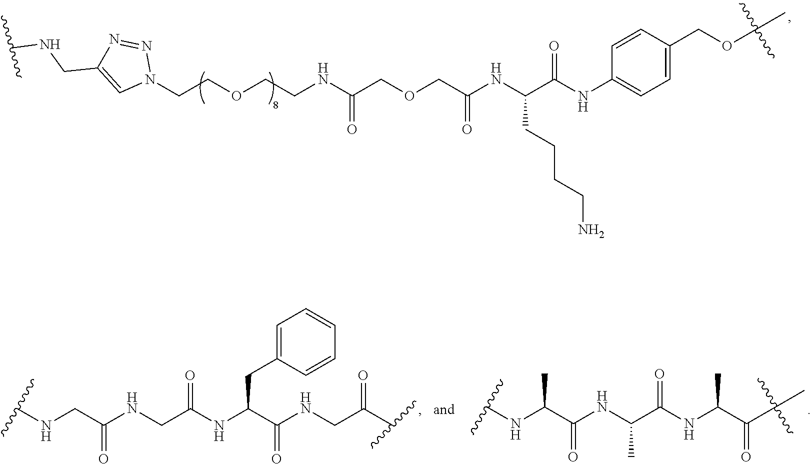

In some embodiments, L is selected from the following structures:

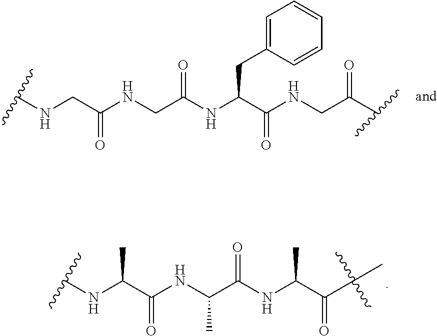 [SMILES: *NCC(=O)NCC(=O)N[C@@H](Cc1ccccc1)C(=O)NCC(*)=O ; *N[C@@H](C)C(=O)N[C@@H](C)C(=O)N[C@@H](C)C(*)=O]

In some embodiments, L is

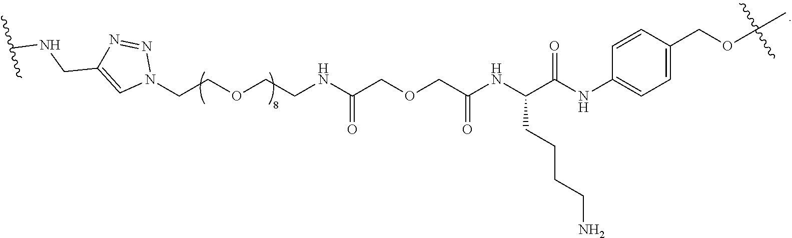

In some embodiments, L is

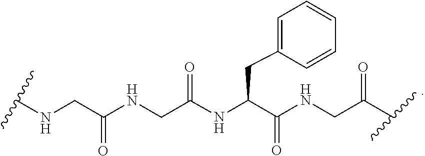 [SMILES: *NCC(=O)NCC(=O)N[C@@H](Cc1ccccc1)C(=O)NCC(*)=O]

In some embodiments, L is

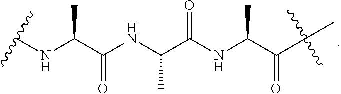 [SMILES: *N[C@@H](C)C(=O)N[C@@H](C)C(=O)N[C@@H](C)C(*)=O]

In some embodiments, E is a single bond, —NH—CH₂—, —NH—CH₂—O—CH₂—CO—,

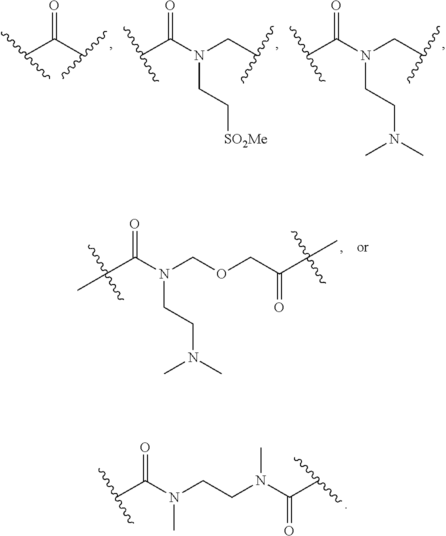

In some embodiments, E is a single bond, —NH—CH₂—, —NH—CH₂—O—CH₂—CO—,

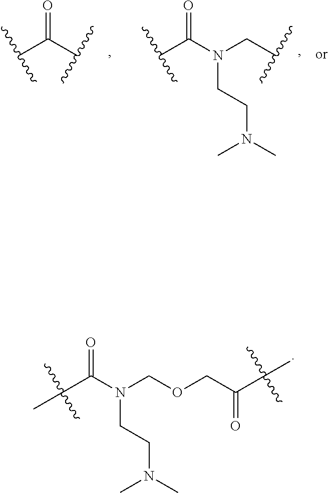

In some embodiments, E is —NH—CH₂—,

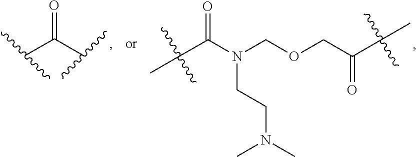

In some embodiments, E is —NH—CH₂—, or

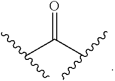 [SMILES: *C(*)=O]

In some embodiments, M is selected from the following structures:

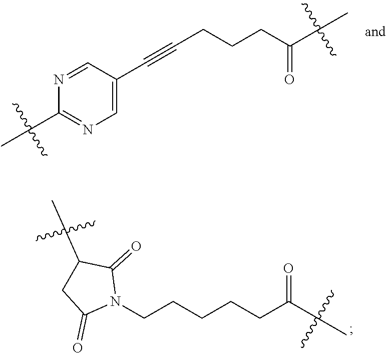 [SMILES: *C(=O)CCCC#Cc1cnc(*)nc1 ; *C(=O)CCCCCN1C(=O)CC(*)C1=O]

and
L is selected from the following structures:

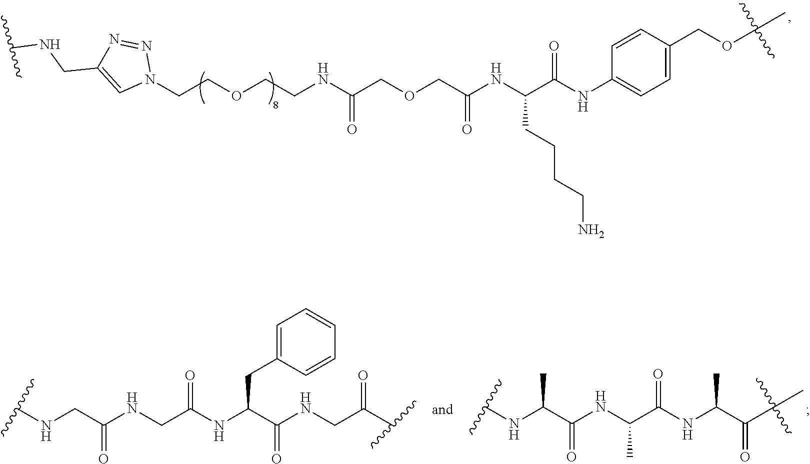

and
E is —NH—CH₂,

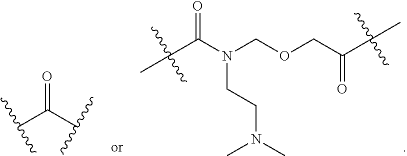

In some embodiments,

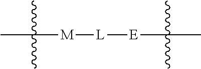

is selected from the following structures:

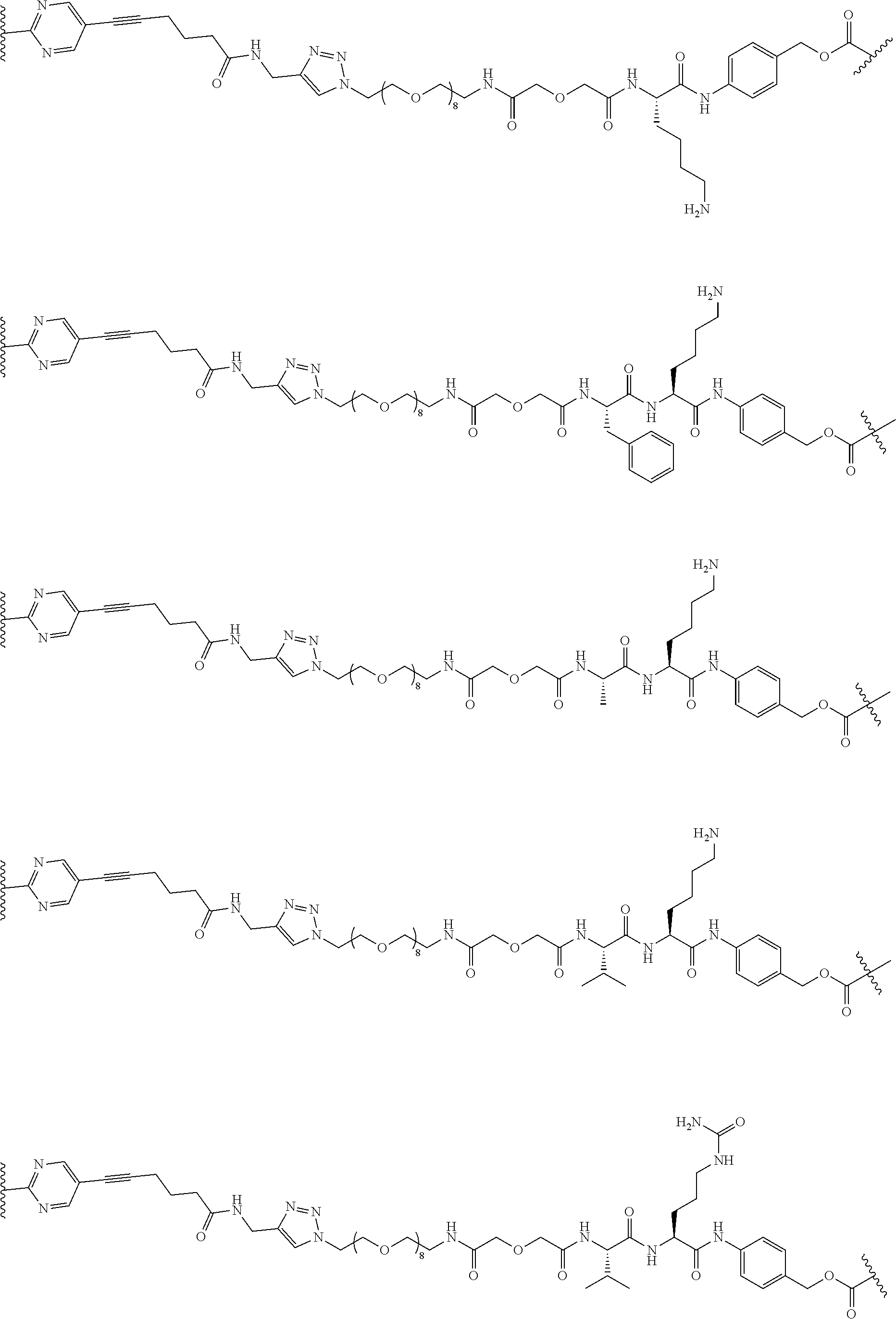

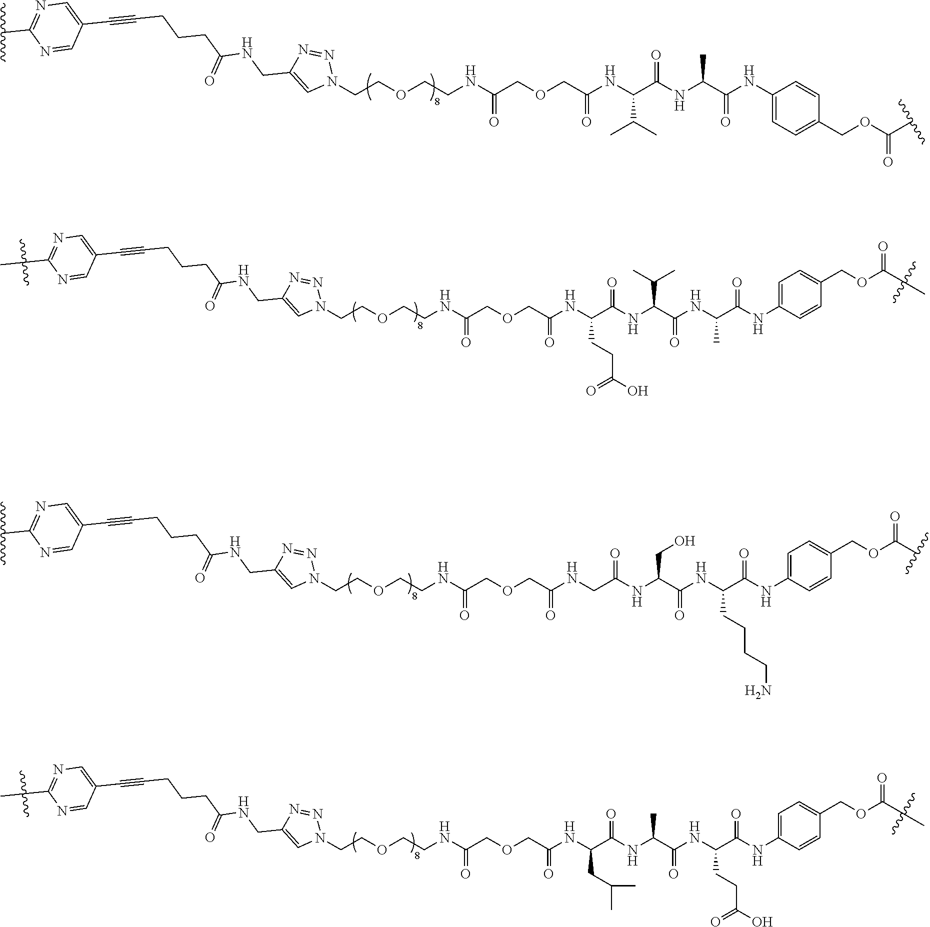

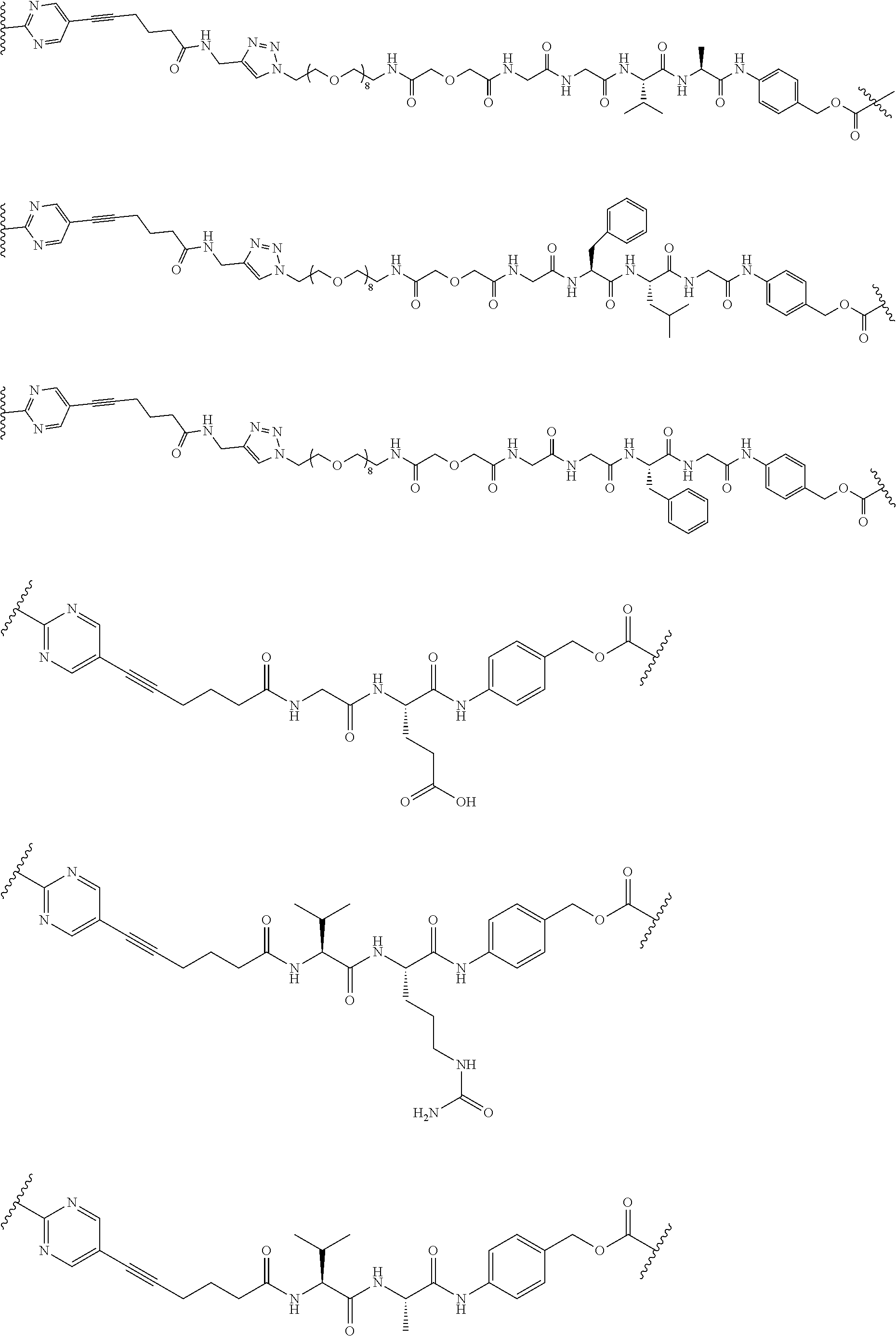

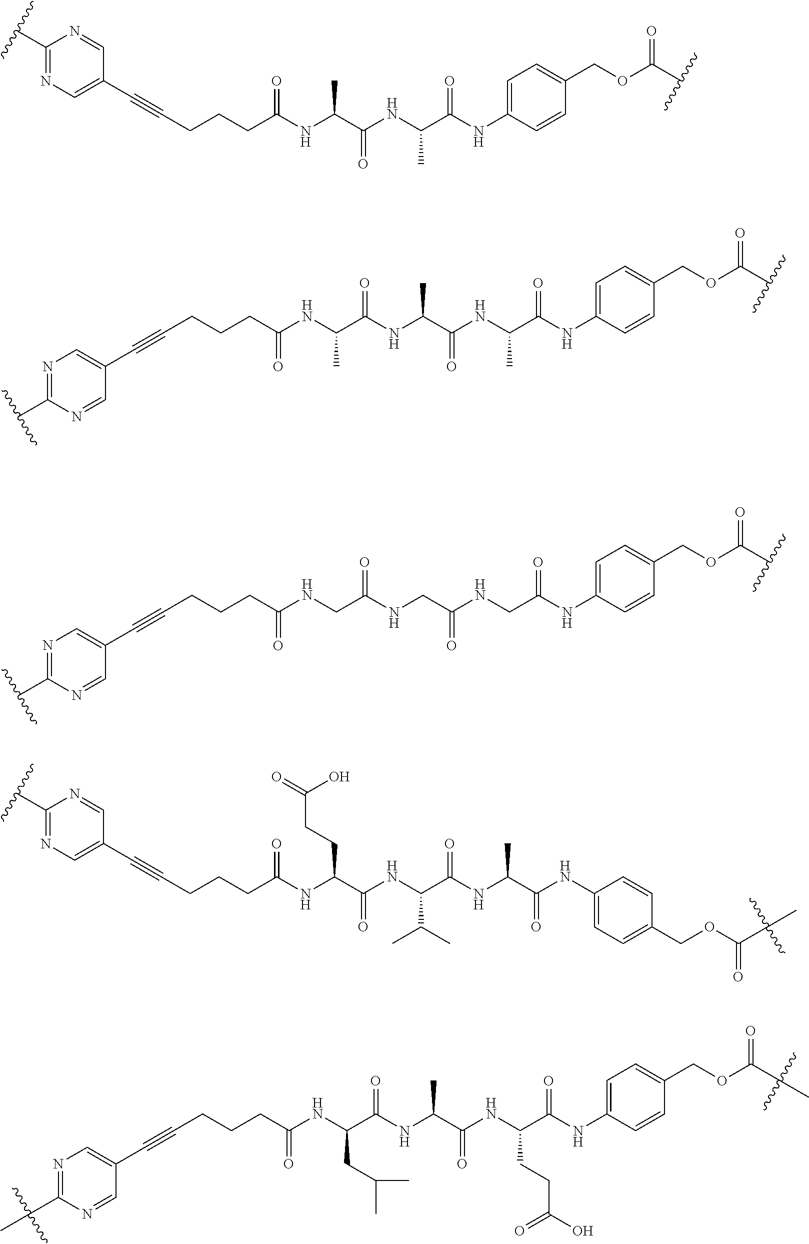 [SMILES: *C(=O)OCc1ccc(NC(=O)[C@H](C)NC(=O)[C@H](C)NC(=O)CCCC#Cc2cnc(*)nc2)cc1 ; *C(=O)OCc1ccc(NC(=O)[C@H](C)NC(=O)[C@H](C)NC(=O)[C@H](C)NC(=O)CCCC#Cc2cnc(*)nc2)cc1 ; *C(=O)OCc1ccc(NC(=O)CNC(=O)CNC(=O)CNC(=O)CCCC#Cc2cnc(*)nc2)cc1 ; *C(=O)OCc1ccc(NC(=O)[C@H](C)NC(=O)[C@@H](NC(=O)[C@H](CCC(=O)O)NC(=O)CCCC#Cc2cnc(*)nc2)C(C)C)cc1 ; *C(=O)OCc1ccc(NC(=O)[C@H](CCC(=O)O)NC(=O)[C@H](C)NC(=O)[C@@H](CC(C)C)NC(=O)CCCC#Cc2cnc(*)nc2)cc1]

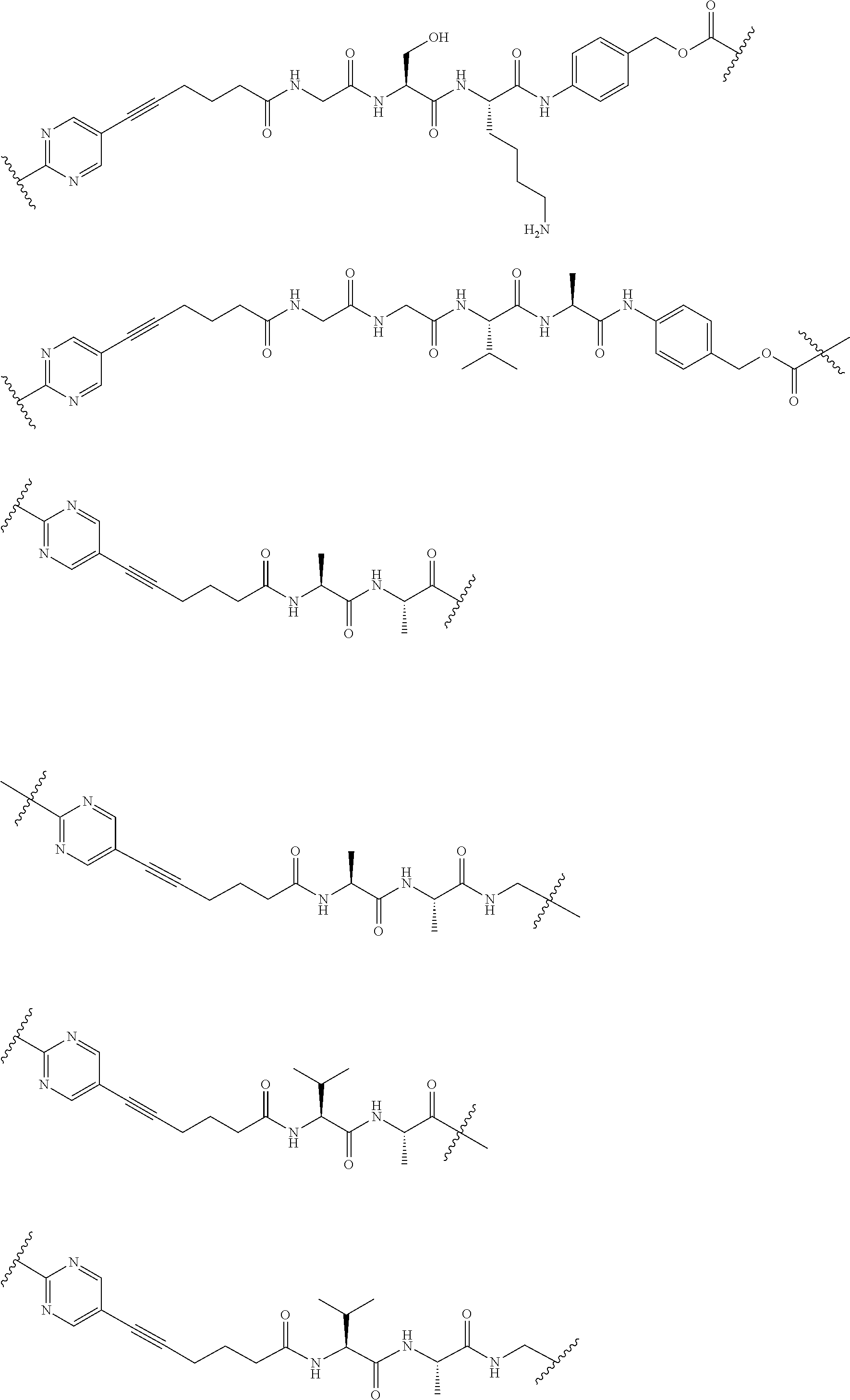 [SMILES: *C(=O)OCc1ccc(NC(=O)[C@H](CCCCN)NC(=O)[C@H](CO)NC(=O)CNC(=O)CCCC#Cc2cnc(*)nc2)cc1 ; *C(=O)OCc1ccc(NC(=O)[C@H](C)NC(=O)[C@@H](NC(=O)CNC(=O)CNC(=O)CCCC#Cc2cnc(*)nc2)C(C)C)cc1 ; *C(=O)[C@H](C)NC(=O)[C@H](C)NC(=O)CCCC#Cc1cnc(*)nc1 ; *CNC(=O)[C@H](C)NC(=O)[C@H](C)NC(=O)CCCC#Cc1cnc(*)nc1 ; *C(=O)[C@H](C)NC(=O)[C@@H](NC(=O)CCCC#Cc1cnc(*)nc1)C(C)C ; *CNC(=O)[C@H](C)NC(=O)[C@@H](NC(=O)CCCC#Cc1cnc(*)nc1)C(C)C]

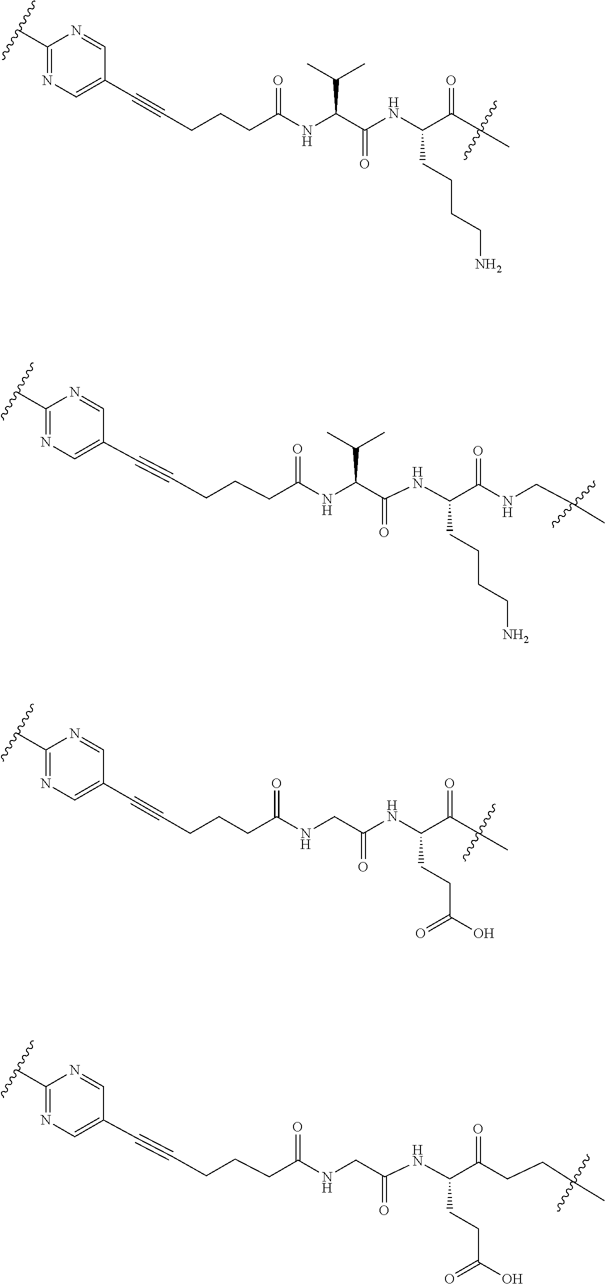 [SMILES: *C(=O)[C@H](CCCCN)NC(=O)[C@@H](NC(=O)CCCC#Cc1cnc(*)nc1)C(C)C ; *CNC(=O)[C@H](CCCCN)NC(=O)[C@@H](NC(=O)CCCC#Cc1cnc(*)nc1)C(C)C ; *C(=O)[C@H](CCC(=O)O)NC(=O)CNC(=O)CCCC#Cc1cnc(*)nc1 ; *CCC(=O)[C@H](CCC(=O)O)NC(=O)CNC(=O)CCCC#Cc1cnc(*)nc1]

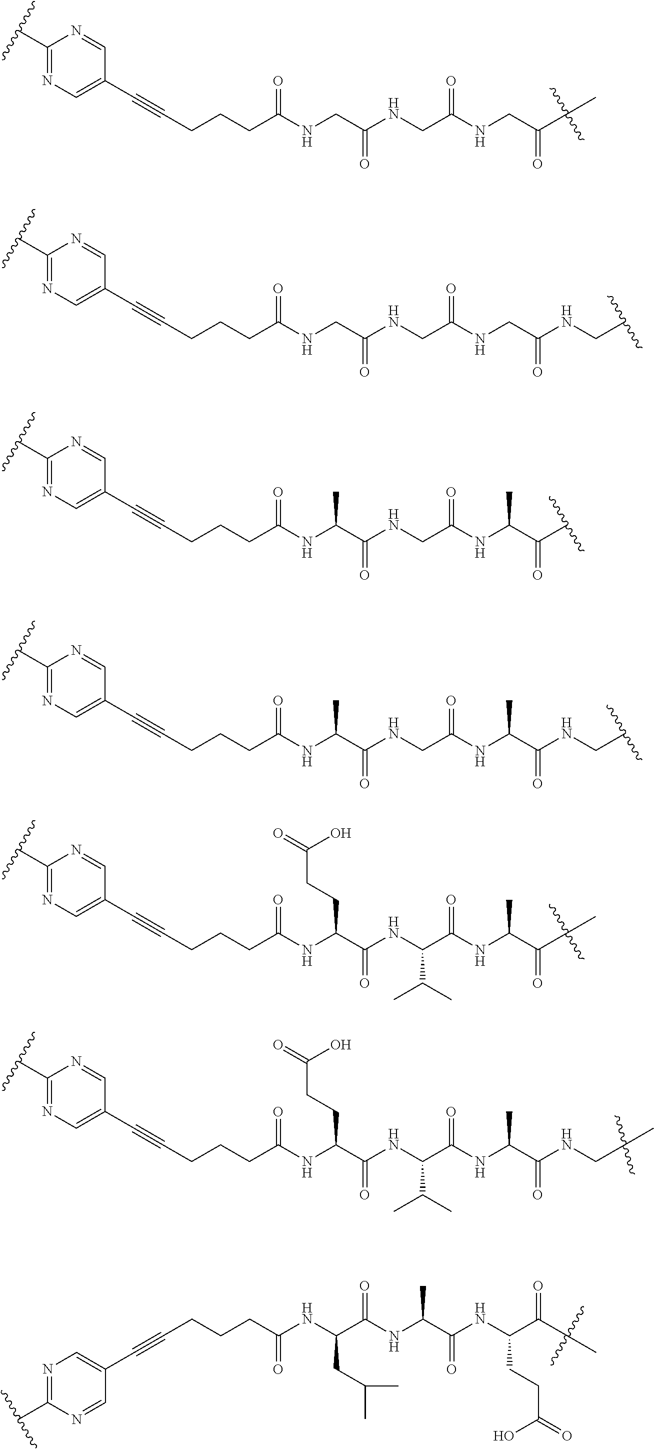

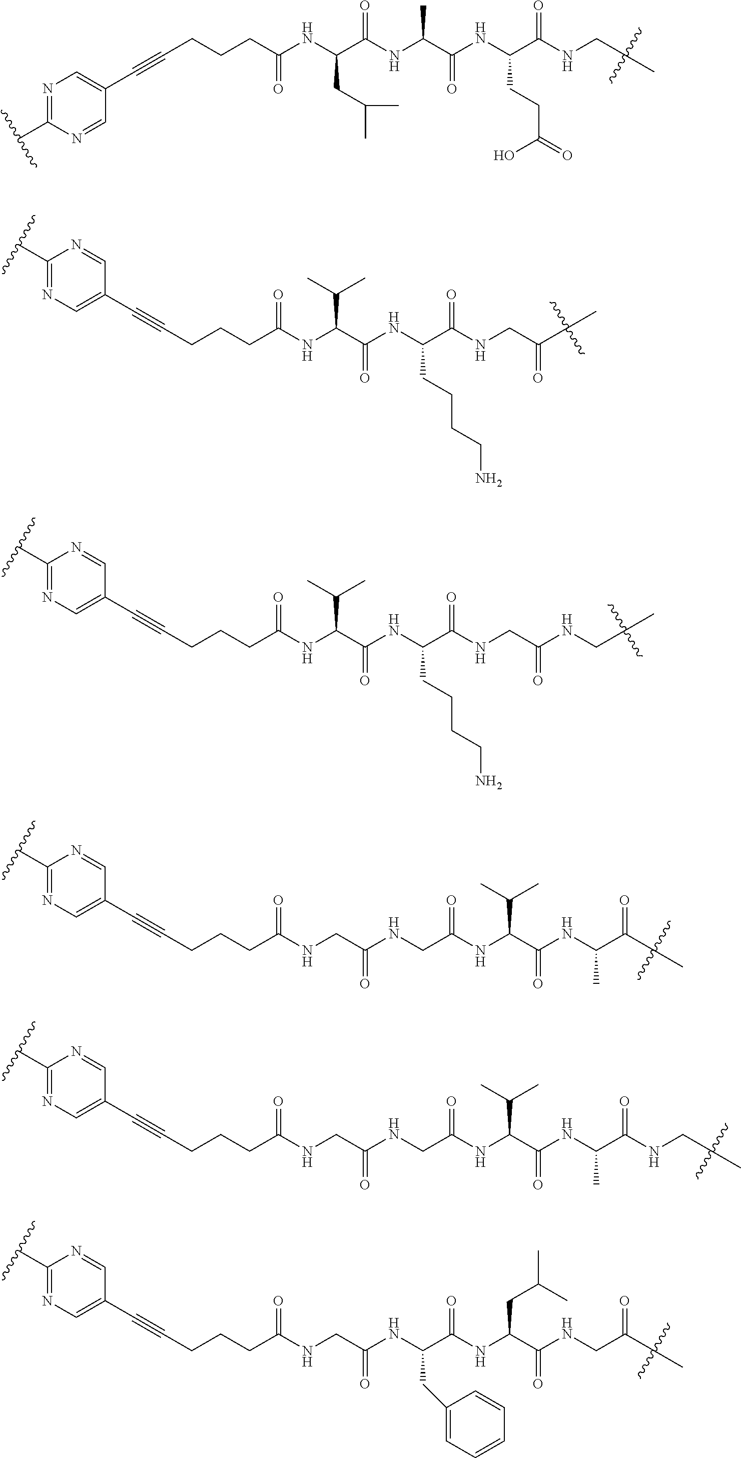 [SMILES: *CNC(=O)[C@H](CCC(=O)O)NC(=O)[C@H](C)NC(=O)[C@@H](CC(C)C)NC(=O)CCCC#Cc1cnc(*)nc1 ; *C(=O)CNC(=O)[C@H](CCCCN)NC(=O)[C@@H](NC(=O)CCCC#Cc1cnc(*)nc1)C(C)C ; *CNC(=O)CNC(=O)[C@H](CCCCN)NC(=O)[C@@H](NC(=O)CCCC#Cc1cnc(*)nc1)C(C)C ; *C(=O)[C@H](C)NC(=O)[C@@H](NC(=O)CNC(=O)CNC(=O)CCCC#Cc1cnc(*)nc1)C(C)C ; *CNC(=O)[C@H](C)NC(=O)[C@@H](NC(=O)CNC(=O)CNC(=O)CCCC#Cc1cnc(*)nc1)C(C)C ; *C(=O)CNC(=O)[C@H](CC(C)C)NC(=O)[C@H](Cc1ccccc1)NC(=O)CNC(=O)CCCC#Cc1cnc(*)nc1]

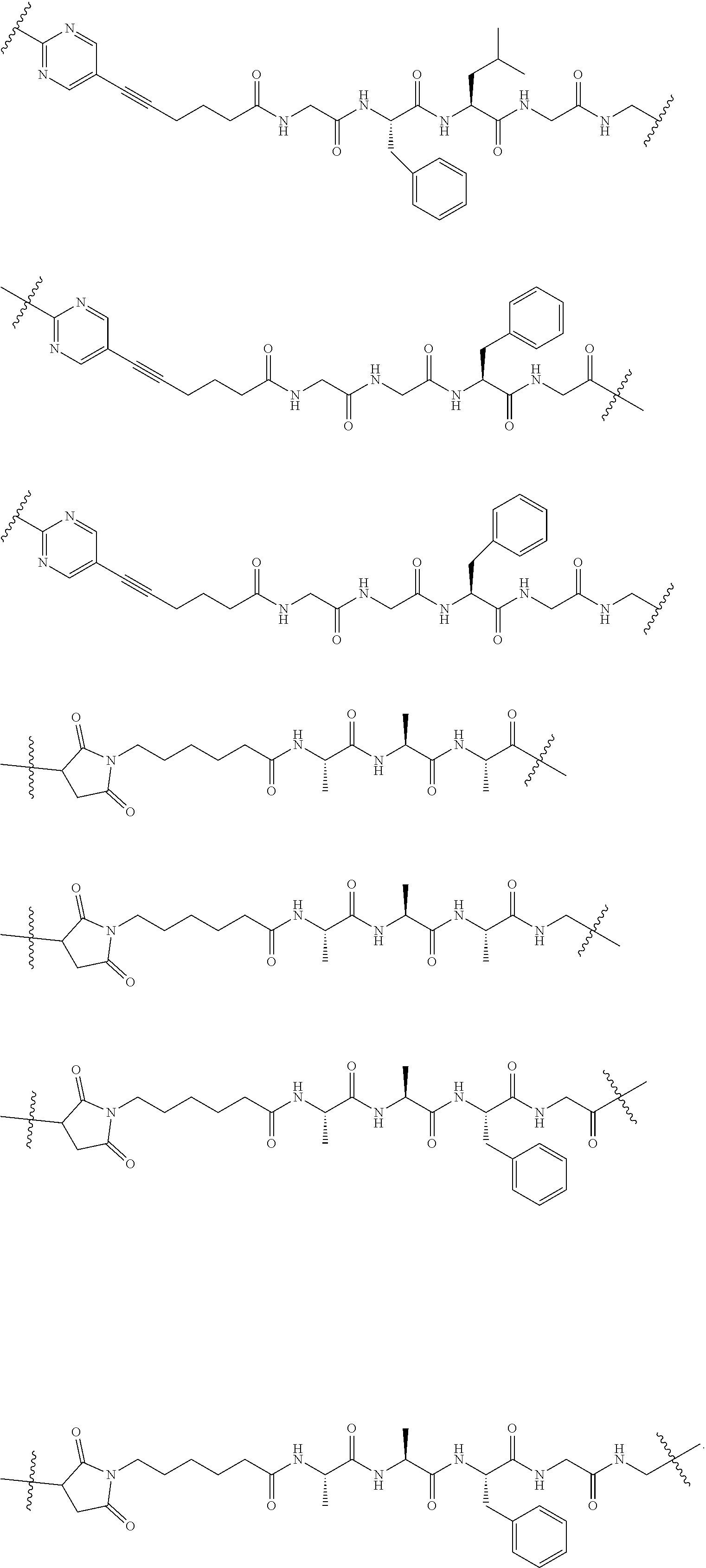

In some embodiments,

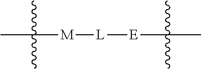

is selected from the following structures:

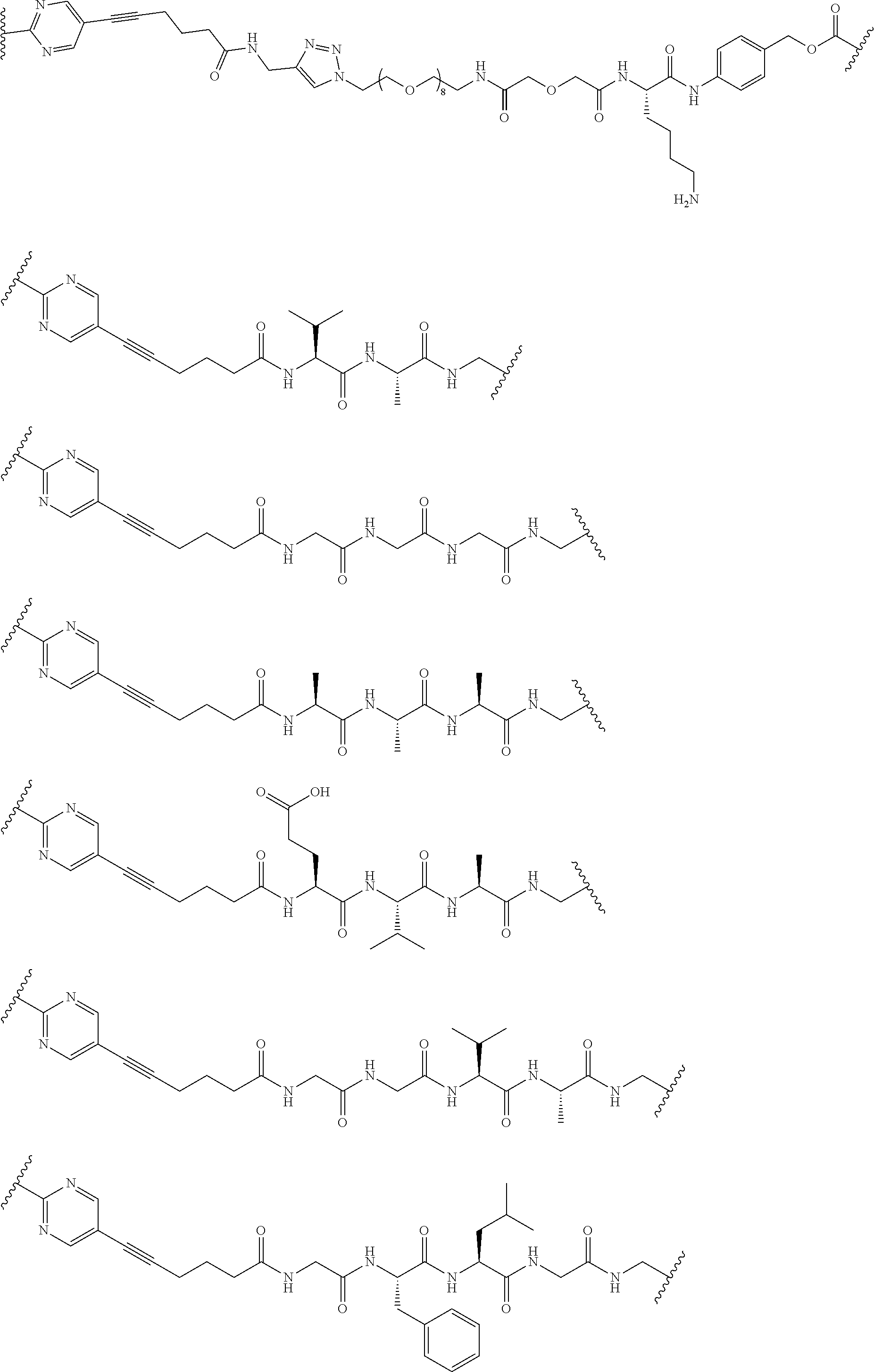

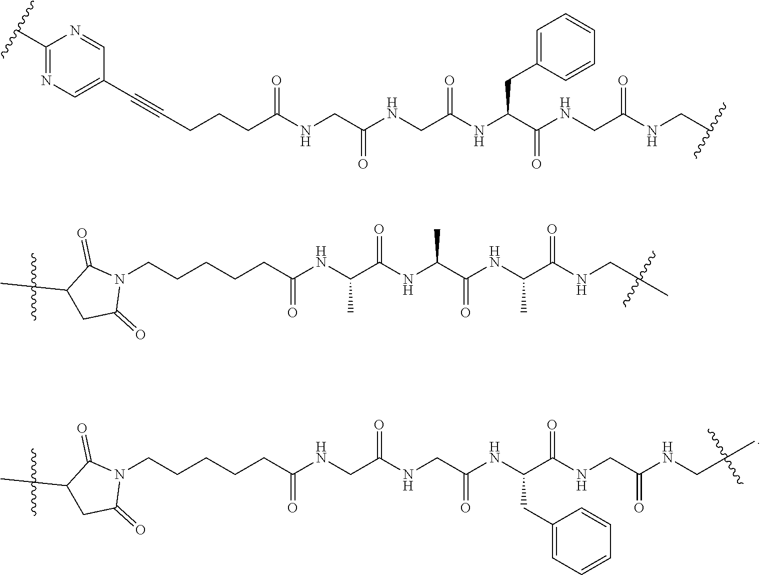 [SMILES: *CNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCCCN1C(=O)CC(*)C1=O ; *CNC(=O)CNC(=O)[C@H](Cc1ccccc1)NC(=O)CNC(=O)CNC(=O)CCCCCN1C(=O)CC(*)C1=O]

In some embodiments,

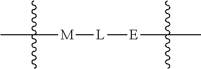

is selected from the following structures:

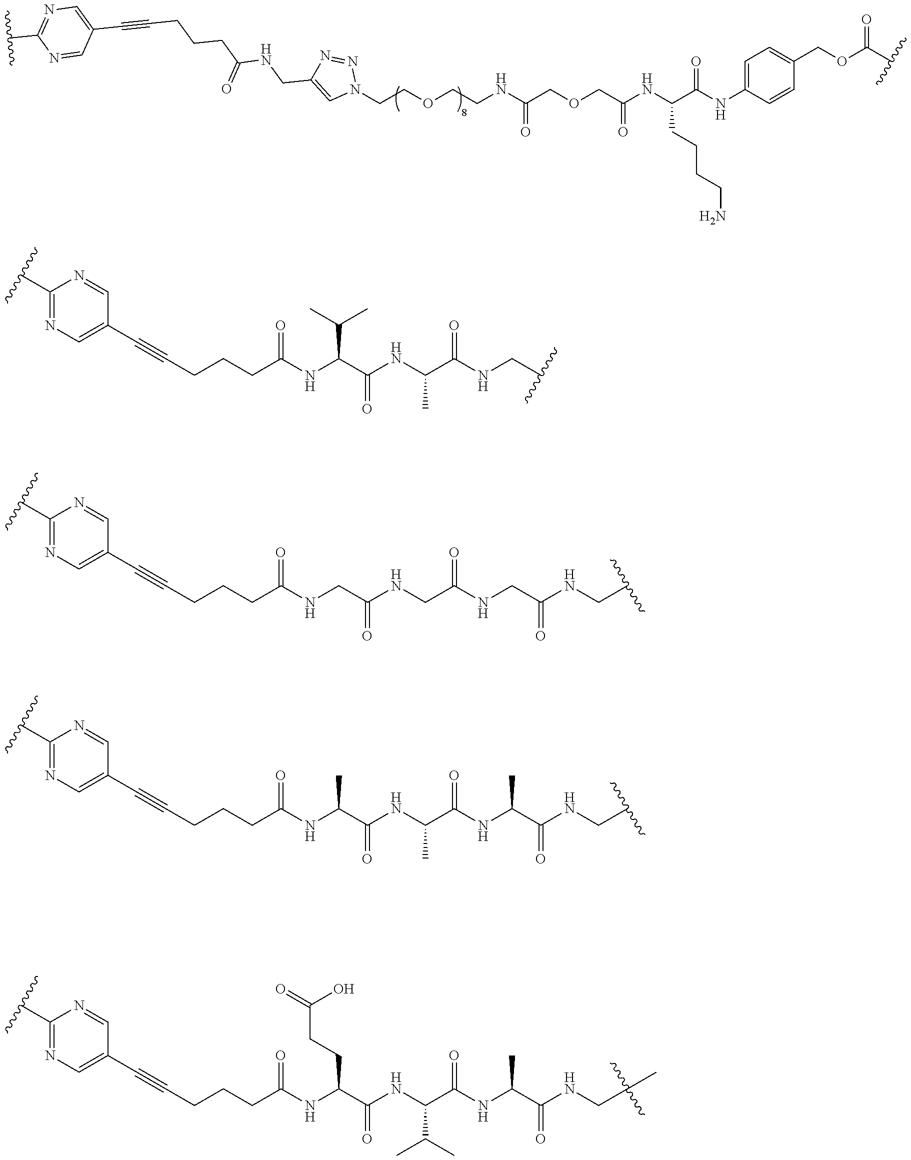

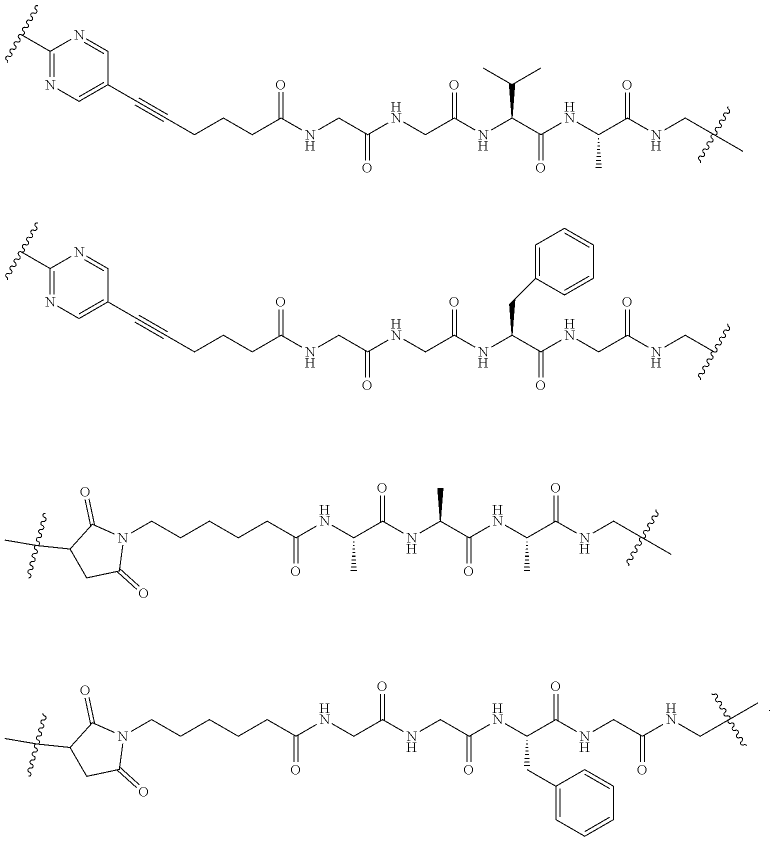 [SMILES: *CNC(=O)[C@H](C)NC(=O)[C@@H](NC(=O)CNC(=O)CNC(=O)CCCC#Cc1cnc(*)nc1)C(C)C ; *CNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCCCN1C(=O)CC(*)C1=O ; *CNC(=O)CNC(=O)[C@H](Cc1ccccc1)NC(=O)CNC(=O)CNC(=O)CCCCCN1C(=O)CC(*)C1=O]

In some embodiments,

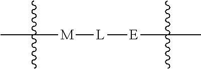

is selected from the following structures:

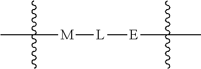

In some embodiments, the cytotoxic drug of an ADC provided herein is selected from a tubulin inhibitor, a DNA intercalator, a DNA topoisomerase inhibitor and an RNA polymerase inhibitor. In some embodiments, the tubulin inhibitor is an auristatin compound or a maytansine compound. In some embodiments, the DNA intercalator is pyrrolobenzodiazepine (PBD). In some embodiments, the DNA topoisomerase inhibitor is a topoisomerase I inhibitor (for example, camptothecin, hydroxycamptothecin, 9-aminocamptothecin, SN-38, irinotecan, topotecan, belotecan, or rubitecan) or a topoisomerase II inhibitor (for example, doxorubicin, PNU-159682, duocarmycin, daunorubicin, mitoxantrone, podophyllotoxin, or etoposide). In some embodiments, the RNA polymerase inhibitor is α-amanitin or a pharmaceutically acceptable salt, an ester or an analog thereof.

Cytotoxic drugs of the ADCs provided herein can contain a plurality of functional groups, for example, hydroxy (—OH), carboxyl (—COOH), sulfhydryl (—SH), primary amino (—NH₂), secondary amino (—NR_{A}H) or tertiary amino (—NR_{B}R_{C}), wherein R_{A}, R_{B} and R_{C} represent non-hydrogen substituents on N, and the cytotoxic drug can be connected with a linker through these functional groups.

In some embodiments, the cytotoxic drug is connected to E in the antibody-drug conjugate by —OH, —SH, primary amino, secondary amino or tertiary amino thereon.

In some embodiments, the cytotoxic drug is selected from the following formula I and formula II:

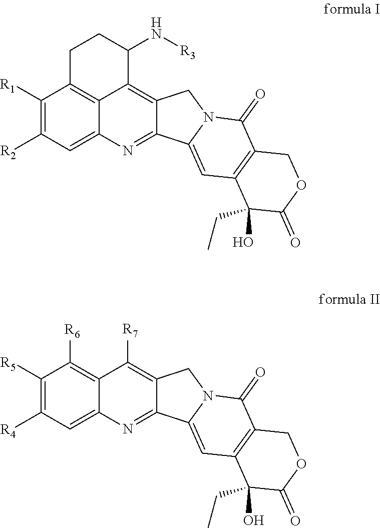

wherein R₁ and R₂ are each independently selected from C₁₋₆alkyl and halogen;
R₃ is selected from H and —CO—CH₂OH;
R₄ and R₅ are each independently selected from H, halogen, and hydroxy; or R₄ and R₅ are taken together with the carbon atom to which they are attached to form a 5-6-membered oxygen-containing heterocyclic ring;
R₆ is selected from hydrogen or —C₁₋₄alkylene-NRₐR_{b}; and
R₇ is selected from hydrogen, C₁₋₆ alkyl, —C₁₋₄ alkylene-NRₐR_{b}, —C₁₋₄ alkylene-SiRₐR_{b}R_{c}, —SiRₐR_{b}R_{c}, —C₁₋₄ alkylene=N—ORₐ; wherein, Rₐ, R_{b} and R_{c} are independently selected from H, C₁₋₆ alkyl, —SO₂—C₁₋₆ alkyl, and —CO—C₁₋₆ alkyl at each occurrence; wherein optionally Rₐ and R_{b} connected to the associated atoms form a 5-6 membered nitrogen containing heterocyclic ring.

In some embodiments, R₇ is selected from hydrogen, C₁₋₆-alkyl, and —C₁₋₄alkylene-NRₐR_{b}; wherein Rₐ and R_{b} are, at each occurrence, each independently selected from H, C₁₋₆alkyl, —SO₂—C₁₋₆alkyl, and —CO—C₁₋₆alkyl. In some embodiments, R₇ is selected from C₁₋₆alkyl and —C₁₋₄alkylene-NRₐR_{b}.

In some embodiments, the cytotoxic drug is selected from the following compounds:

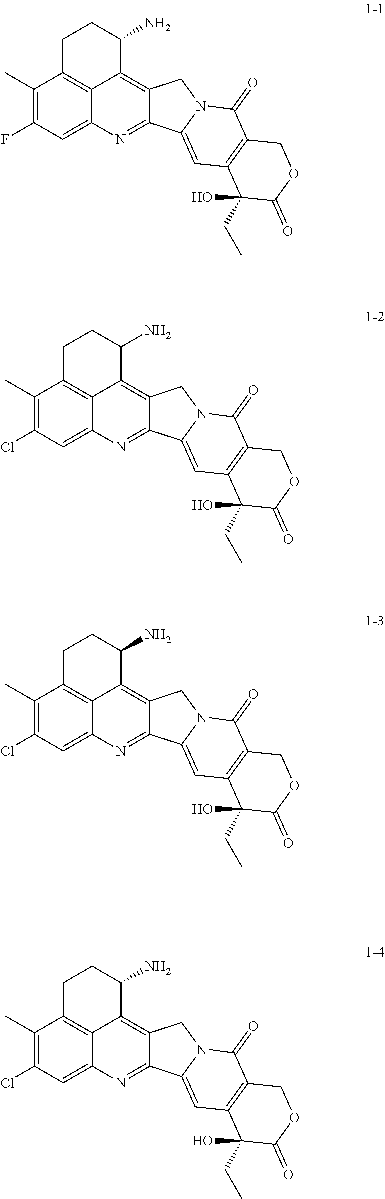 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3]

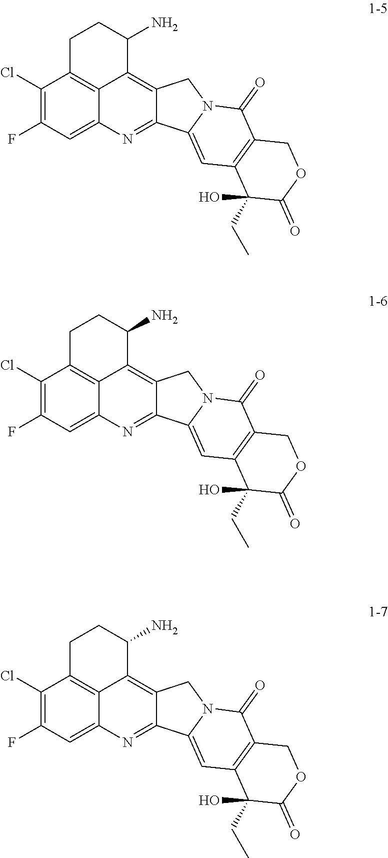 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3]

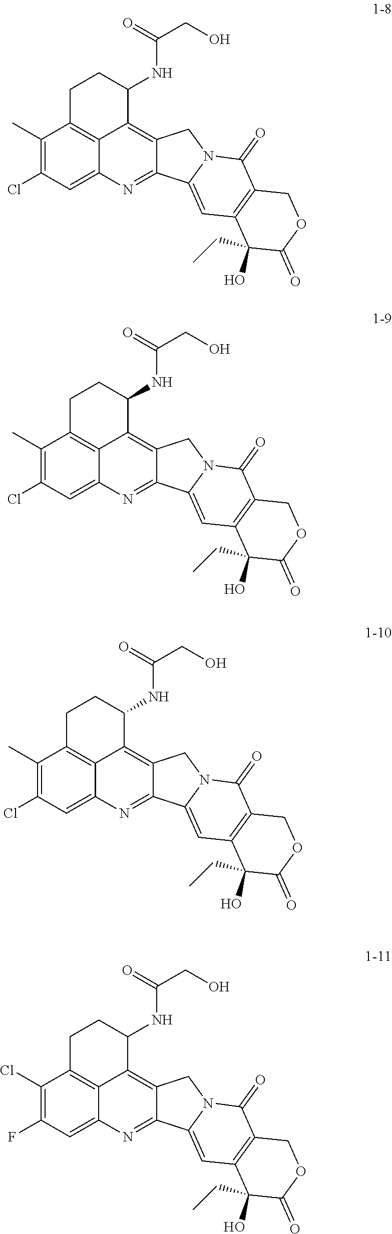 [SMILES: CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3]

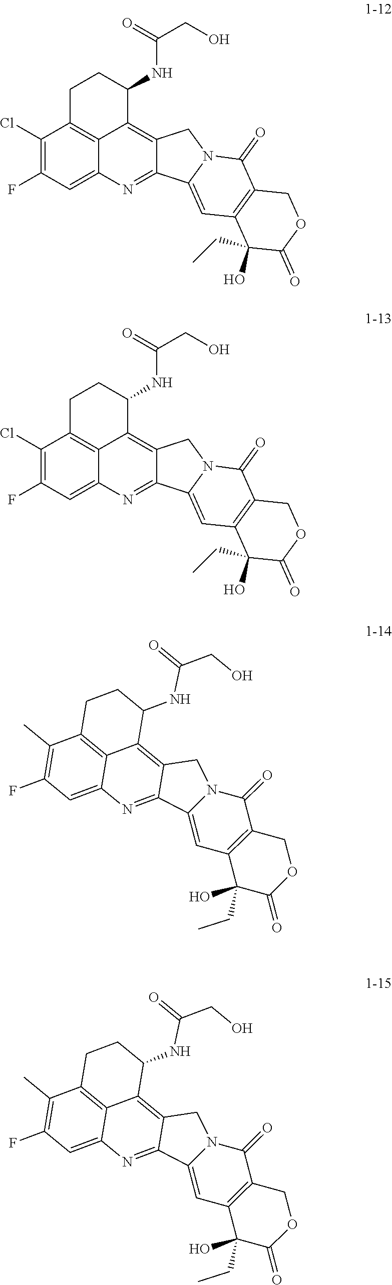 [SMILES: CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2C(NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

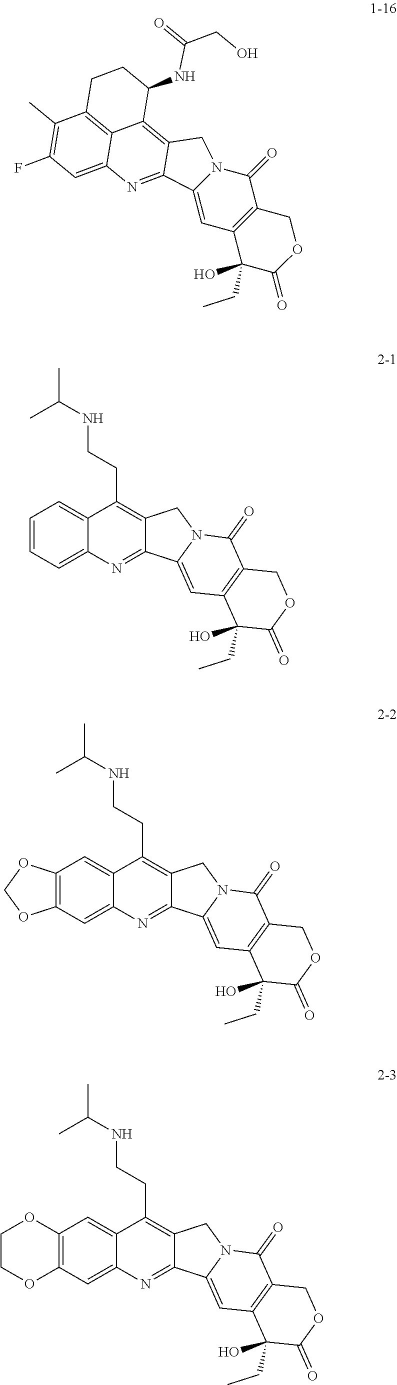 [SMILES: CC[C@@]1(O)C(=O)OCc2c1cc1n(c2=O)Cc2c-1nc1cc(F)c(C)c3c1c2[C@H](NC(=O)CO)CC3 ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3]

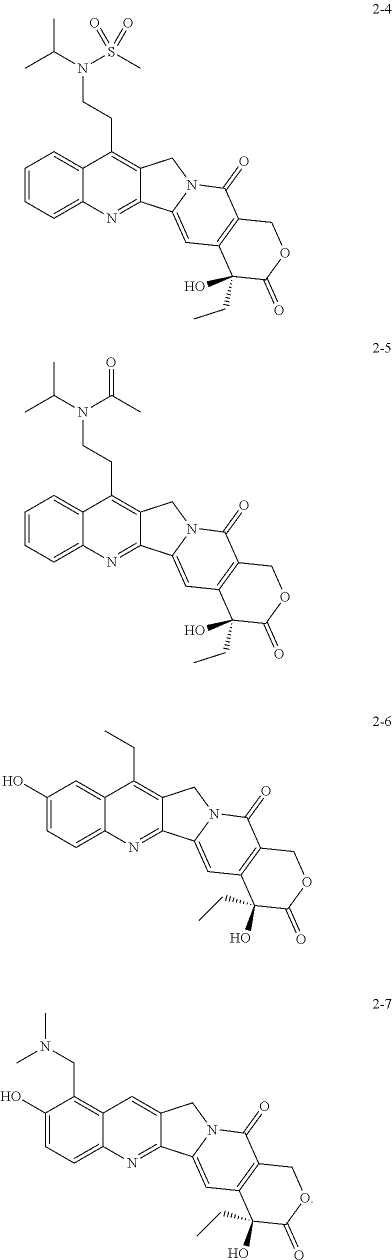 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2cc3c(CN(C)C)c(O)ccc3nc2-1]

In some embodiments, the cytotoxic drug is selected from the following compounds:

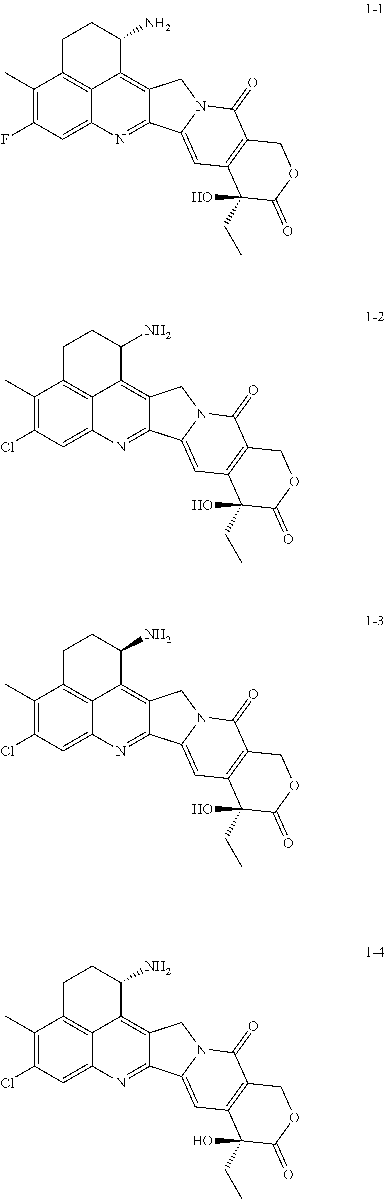 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3]

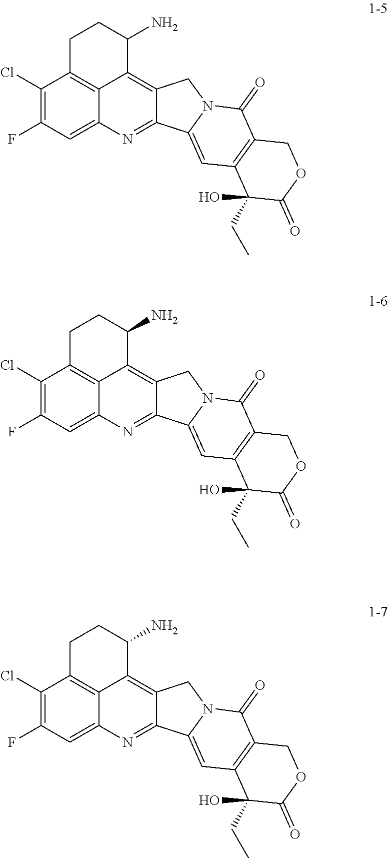 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3]

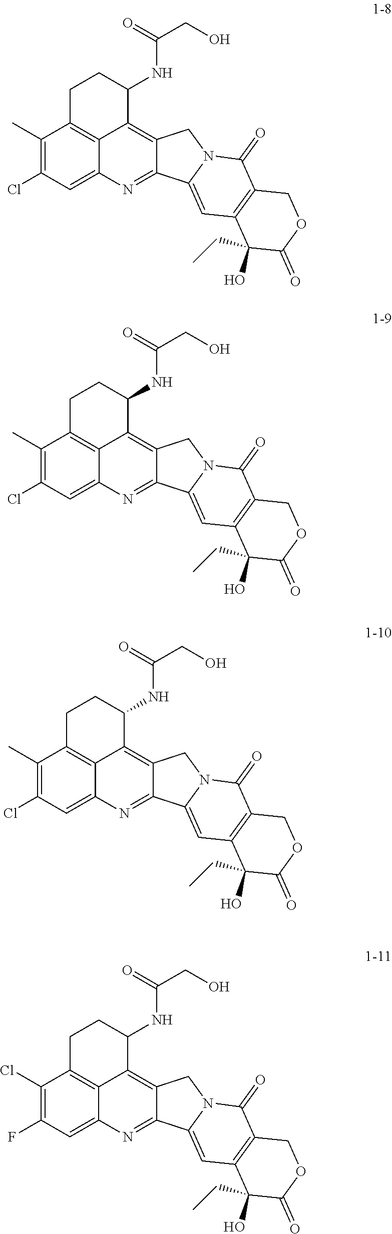 [SMILES: CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3]

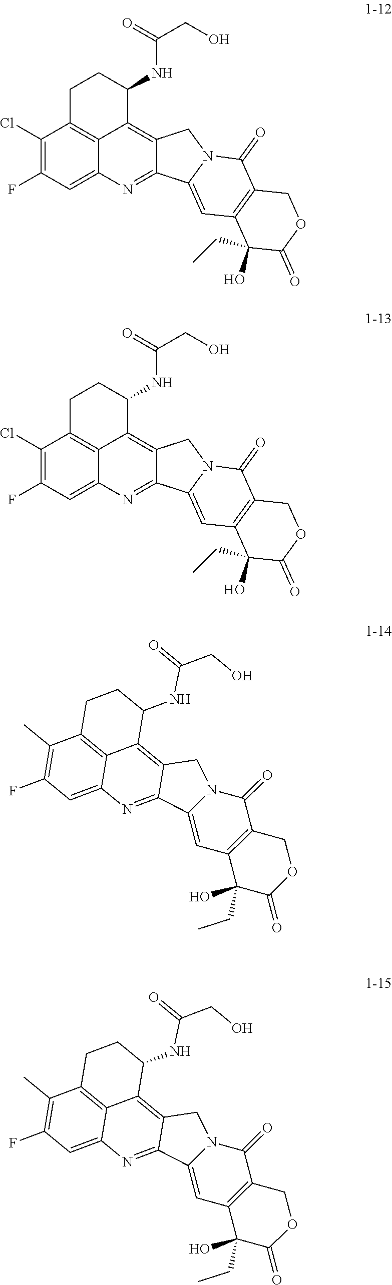 [SMILES: CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2C(NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

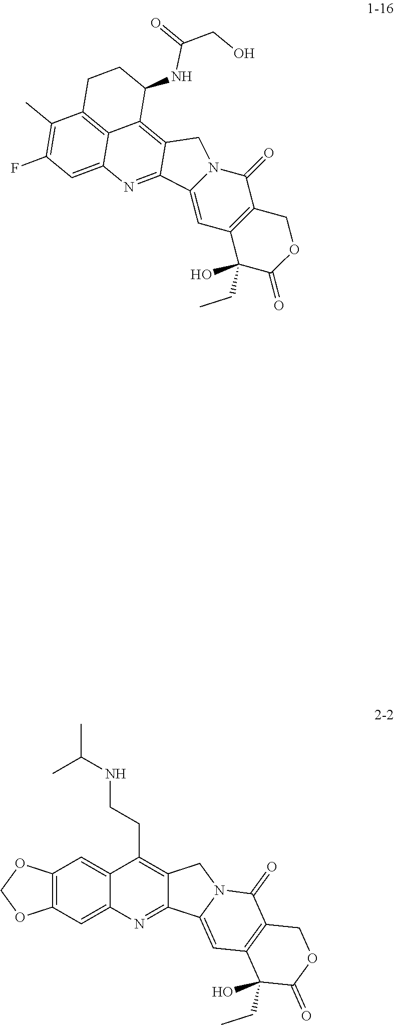 [SMILES: CC[C@@]1(O)C(=O)OCc2c1cc1n(c2=O)Cc2c-1nc1cc(F)c(C)c3c1c2[C@H](NC(=O)CO)CC3 ; CC[C@@]1(O)C(=O)OCc2c1cc1n(c2=O)Cc2c-1nc1cc3c(cc1c2CCNC(C)C)OCO3]

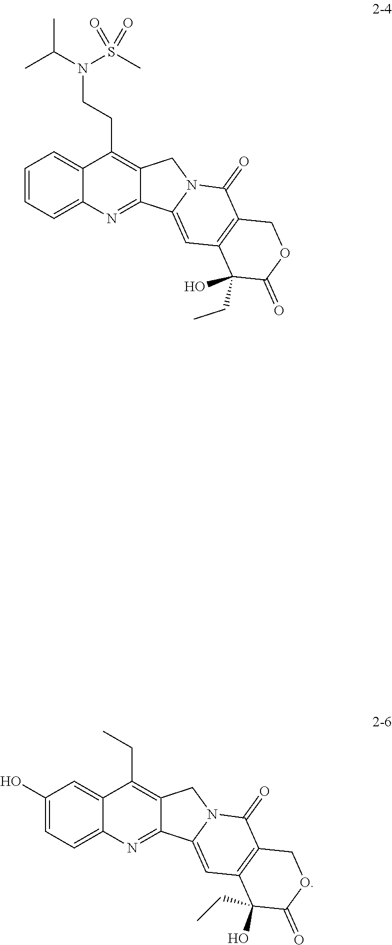 [SMILES: CC[C@@]1(O)C(=O)OCc2c1cc1n(c2=O)Cc2c-1nc1ccccc1c2CCN(C(C)C)S(C)(=O)=O ; CCc1c2c(nc3ccc(O)cc13)-c1cc3c(c(=O)n1C2)COC(=O)[C@]3(O)CC]

In some embodiments, the cytotoxic drug is selected from the following compounds:

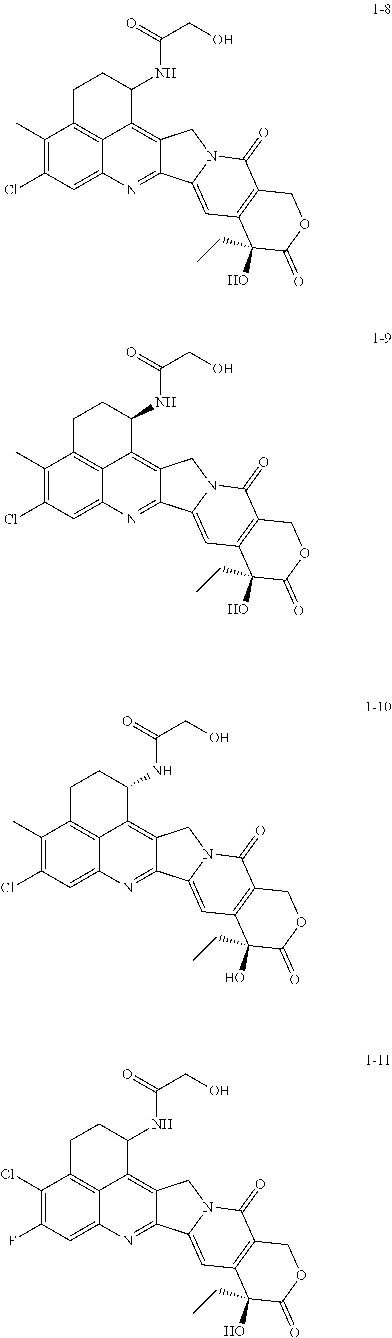 [SMILES: CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3]

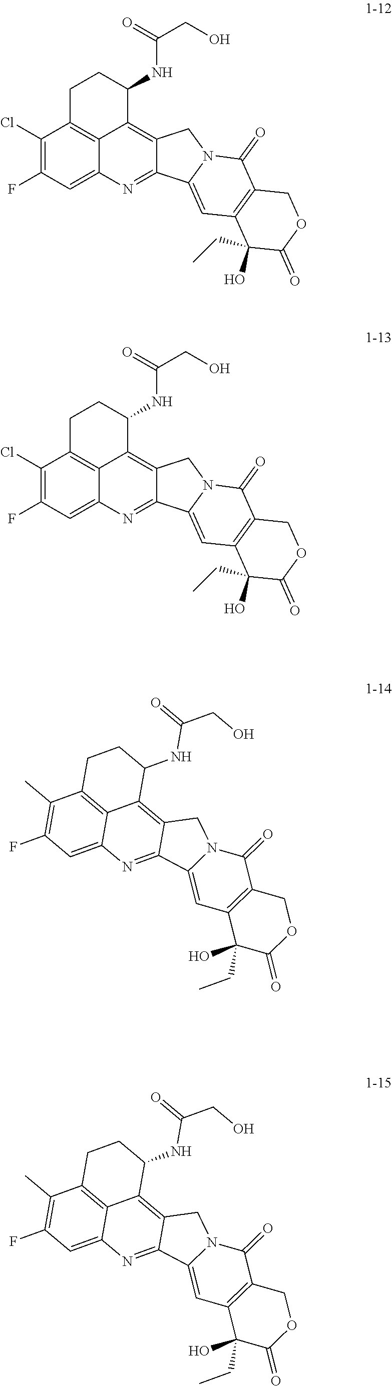 [SMILES: CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2C(NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

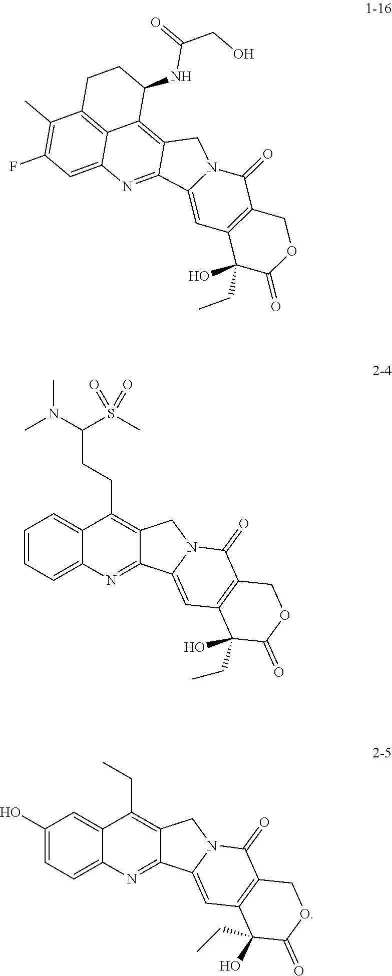 [SMILES: CC[C@@]1(O)C(=O)OCc2c1cc1n(c2=O)Cc2c-1nc1cc(F)c(C)c3c1c2[C@H](NC(=O)CO)CC3 ; CC[C@@]1(O)C(=O)OCc2c1cc1n(c2=O)Cc2c-1nc1ccccc1c2CCC(N(C)C)S(C)(=O)=O ; CCc1c2c(nc3ccc(O)cc13)-c1cc3c(c(=O)n1C2)COC(=O)[C@]3(O)CC]

In some embodiments, the cytotoxic drug is selected from the following compounds:

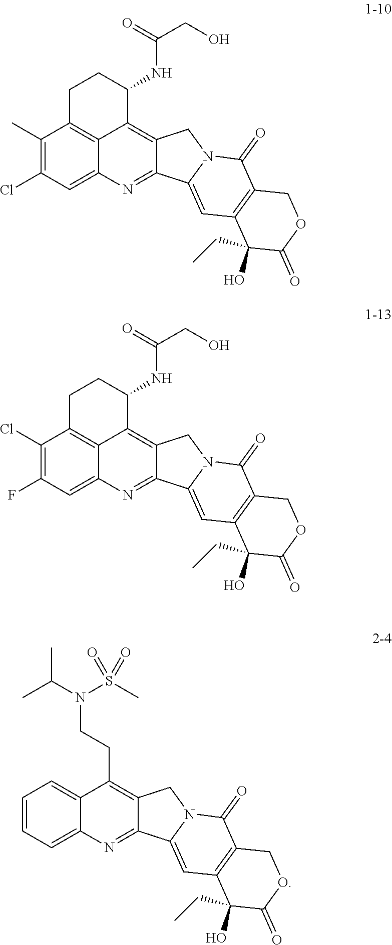 [SMILES: CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCN(C(C)C)S(C)(=O)=O]

In some embodiments, the cytotoxic drug is selected from the following compounds:

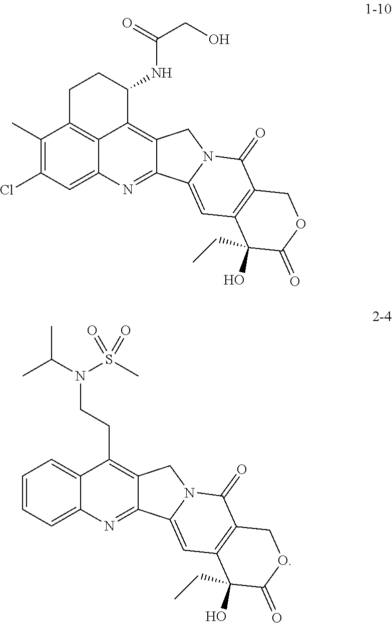 [SMILES: CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1ccccc1c2CCN(C(C)C)S(C)(=O)=O]

A cytotoxic drug moiety obtained after the cytotoxic drug is attached to a linker is D in the formula Ab′-(M-L-E-D)ₓ. of the present disclosure. In some embodiments, D is a cytotoxic drug moiety obtained by losing one H from —OH, —NH₂ or secondary amino on the cytotoxic drug.

In some embodiments, D is selected from the following structures:

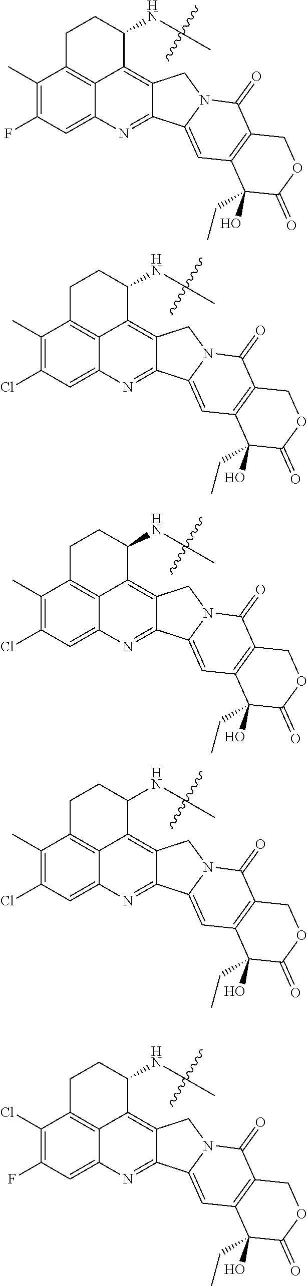 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC ; *N[C@H]1CCc2c(Cl)c(F)cc3nc4c(c1c23)Cn1c-4cc2c(c1=O)COC(=O)[C@]2(O)CC]

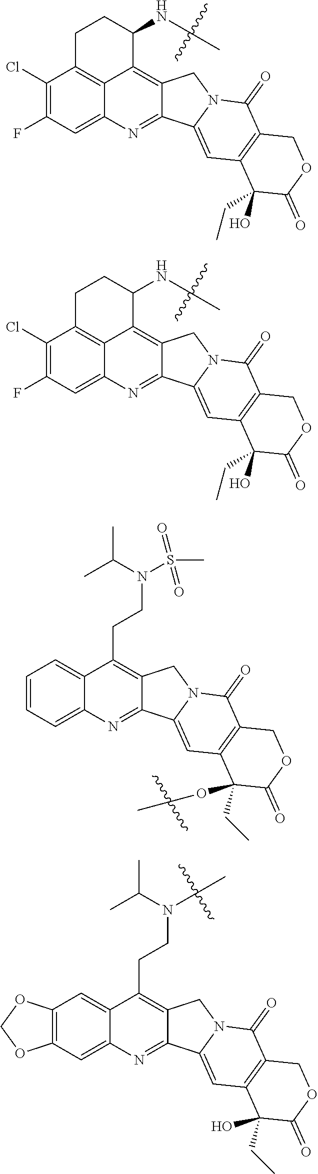 [SMILES: *N[C@@H]1CCc2c(Cl)c(F)cc3nc4c(c1c23)Cn1c-4cc2c(c1=O)COC(=O)[C@]2(O)CC ; *NC1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O ; *N(CCc1c2c(nc3cc4c(cc13)OCO4)-c1cc3c(c(=O)n1C2)COC(=O)[C@]3(O)CC)C(C)C]

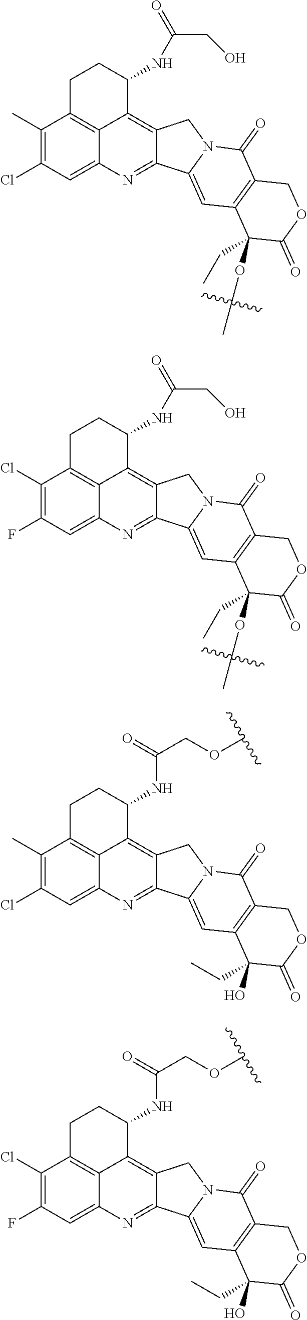 [SMILES: *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3 ; *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC]

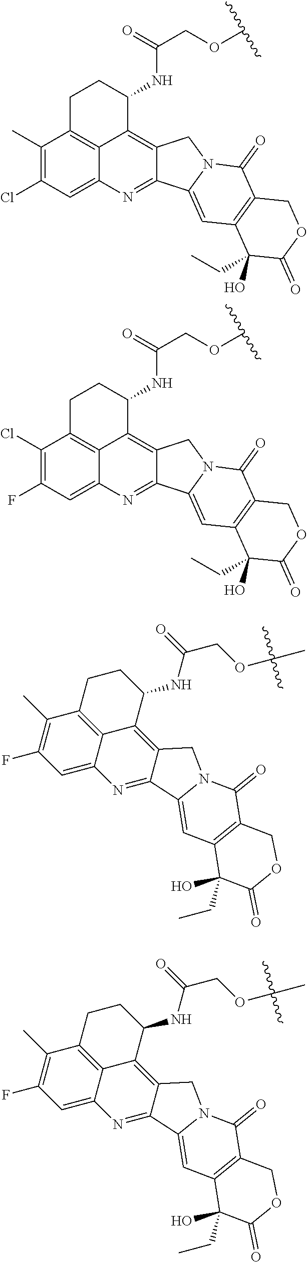

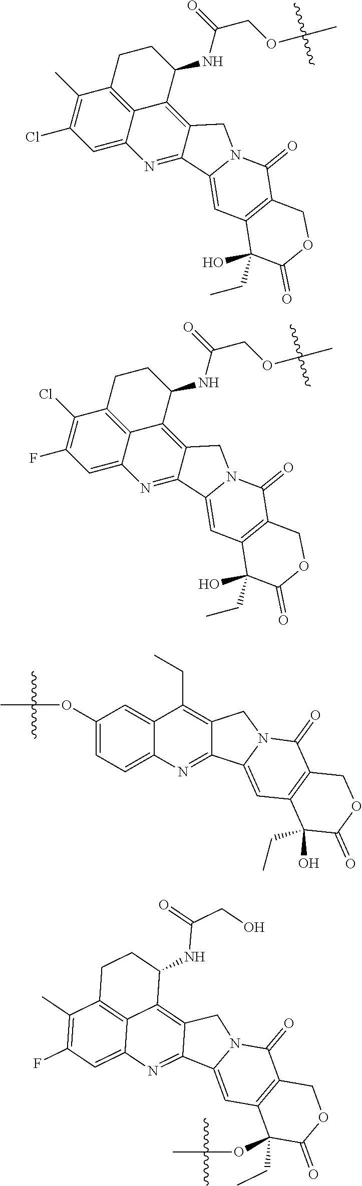

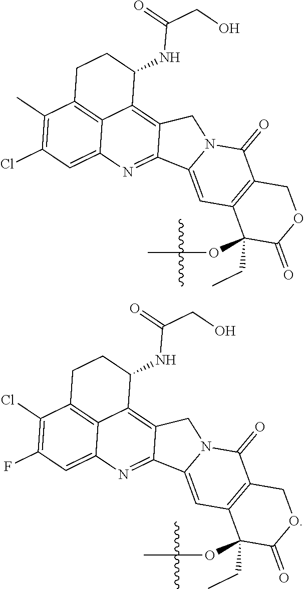

In some embodiments, D is selected from the following structures:

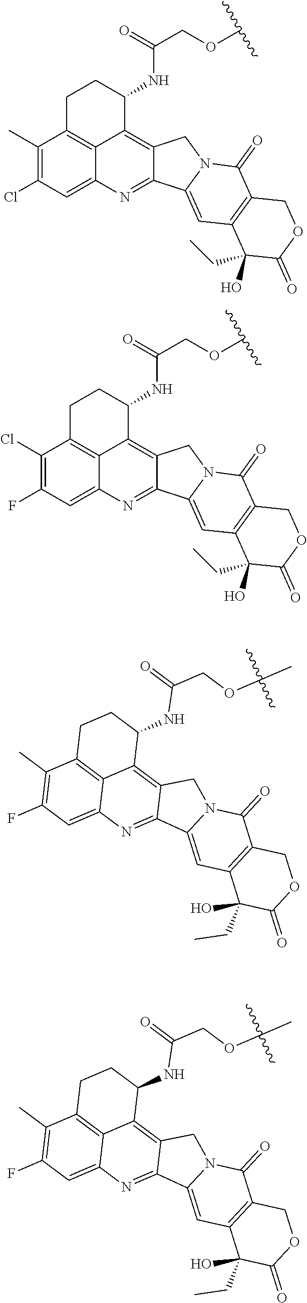 [SMILES: *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *OCC(=O)N[C@@H]1CCc2c(C)c(F)cc3nc4c(c1c23)Cn1c-4cc2c(c1=O)COC(=O)[C@]2(O)CC]

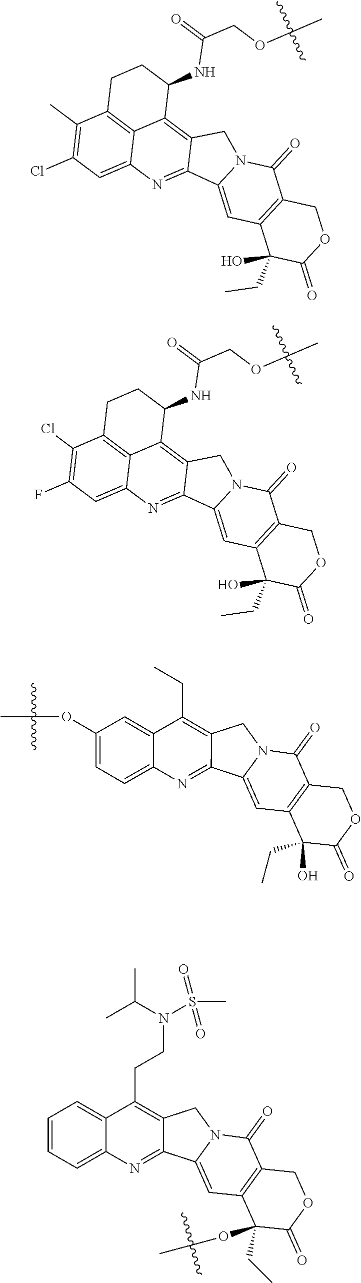 [SMILES: *OCC(=O)N[C@@H]1CCc2c(C)c(Cl)cc3nc4c(c1c23)Cn1c-4cc2c(c1=O)COC(=O)[C@]2(O)CC ; *OCC(=O)N[C@@H]1CCc2c(Cl)c(F)cc3nc4c(c1c23)Cn1c-4cc2c(c1=O)COC(=O)[C@]2(O)CC ; *Oc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O]

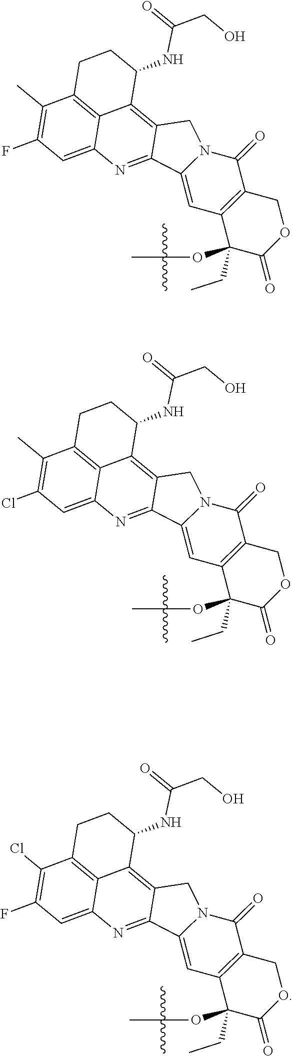 [SMILES: *O[C@]1(CC)C(=O)OCc2c1cc1n(c2=O)Cc2c-1nc1cc(F)c(C)c3c1c2[C@@H](NC(=O)CO)CC3 ; *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3]

In some embodiments, D is selected from the following structures:

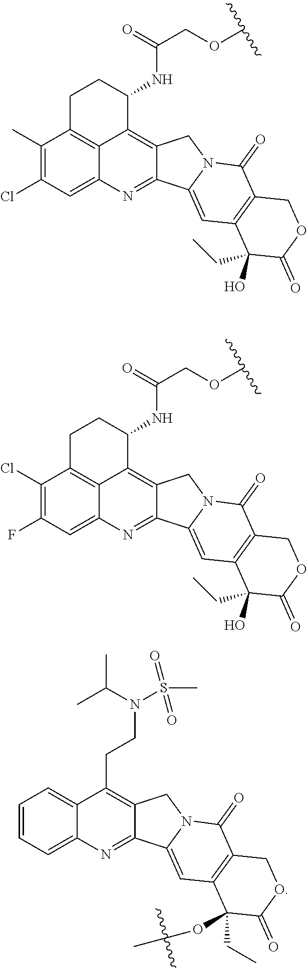 [SMILES: *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O]

In some embodiments, D is selected from the following structures:

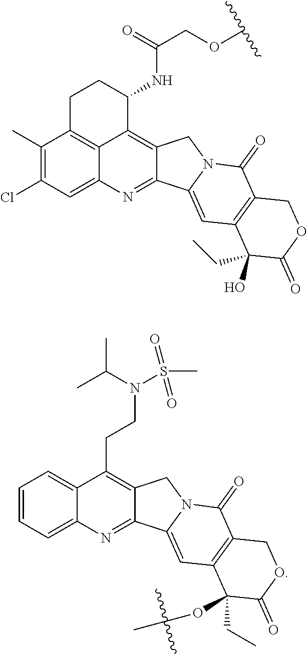 [SMILES: *OCC(=O)N[C@H]1CCc2c(C)c(Cl)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O]

### Exemplary ADCs

In some embodiments, an antibody-drug conjugate provided herein is selected from ADC A-01 to ADC A-34 and ADC B-01 to ADC B-07 as shown below:

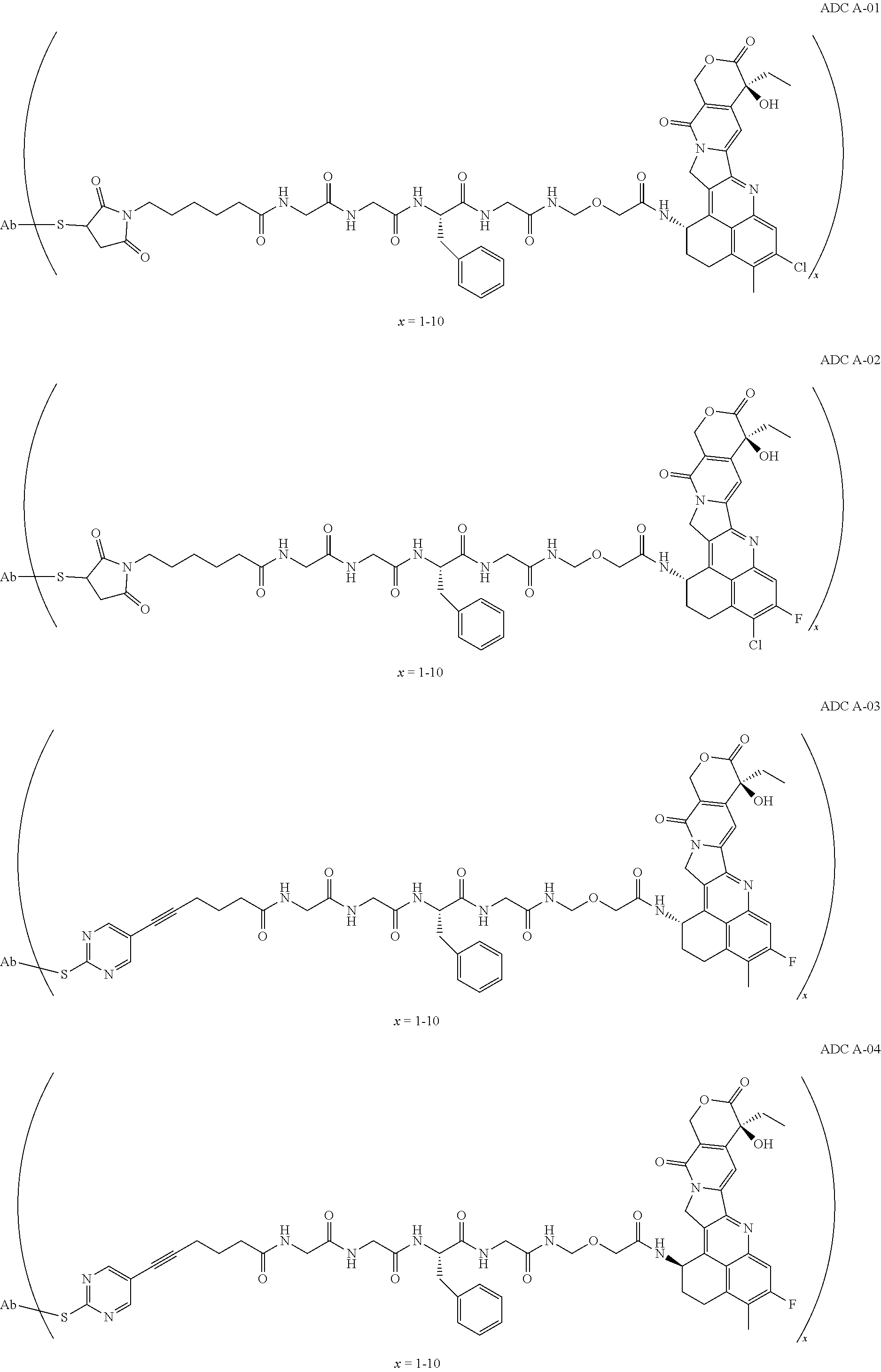

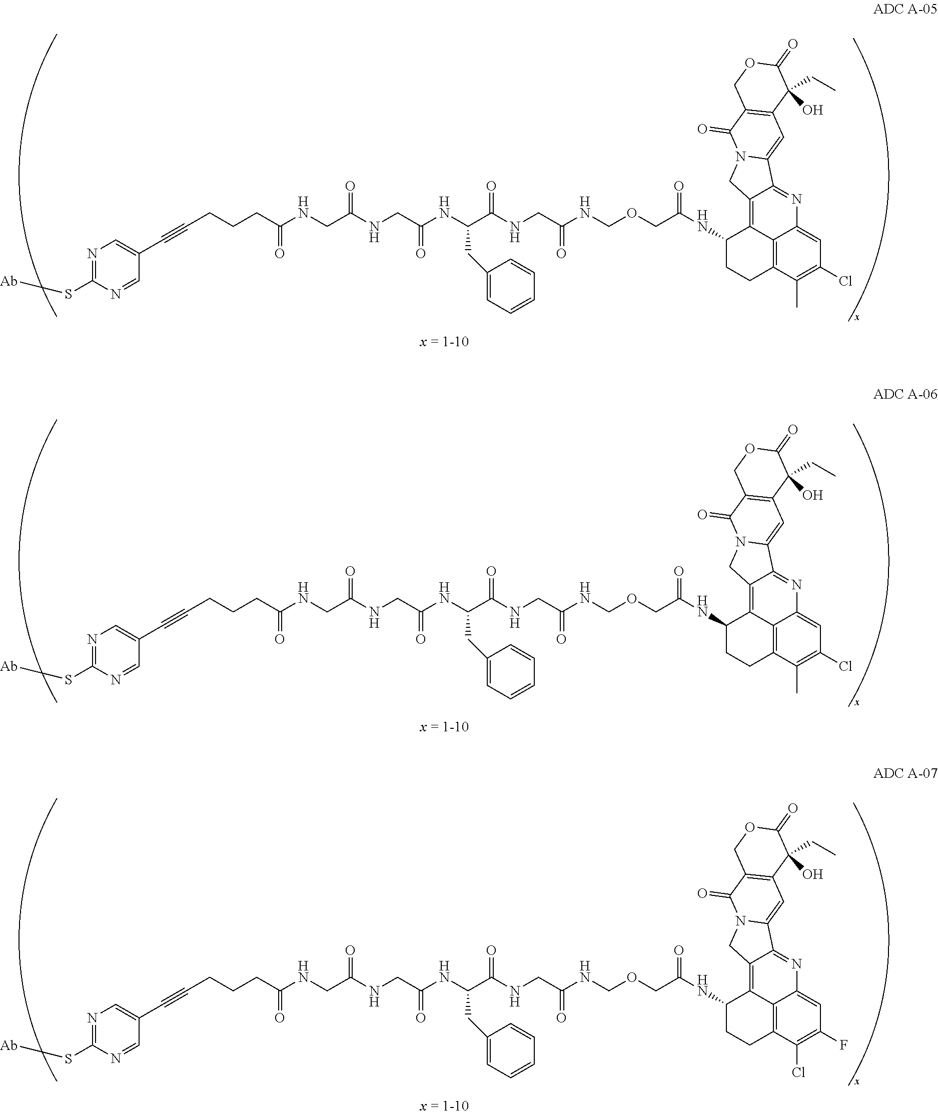

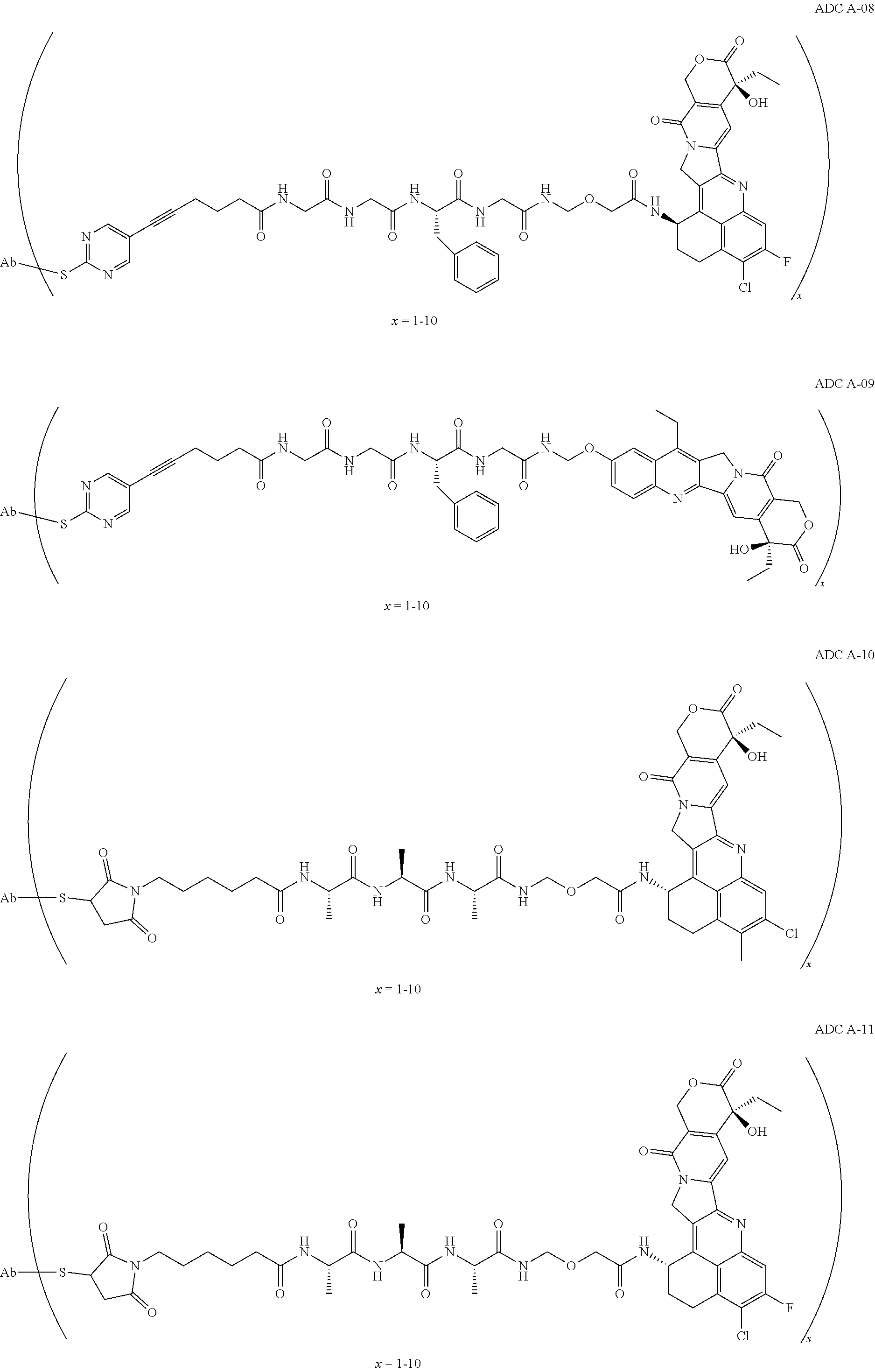

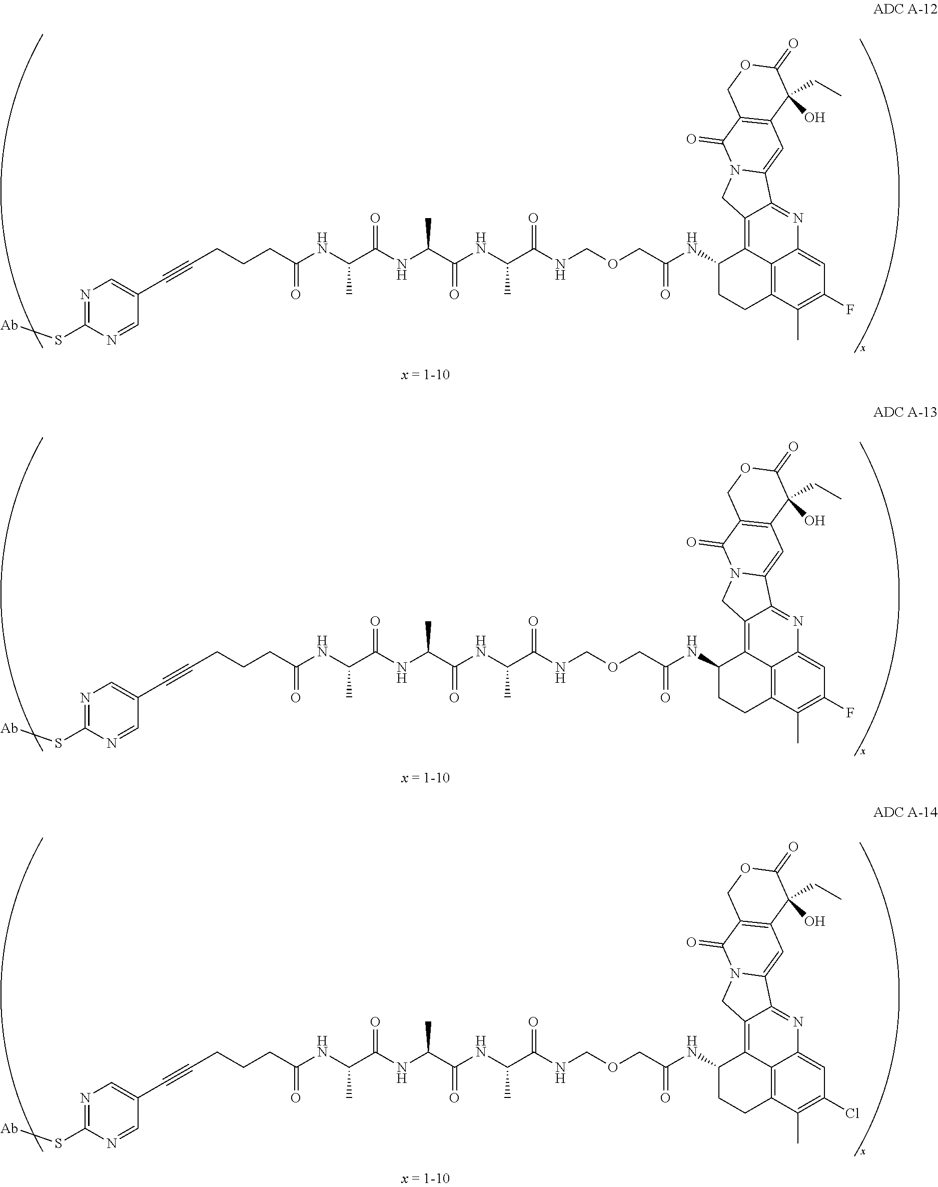

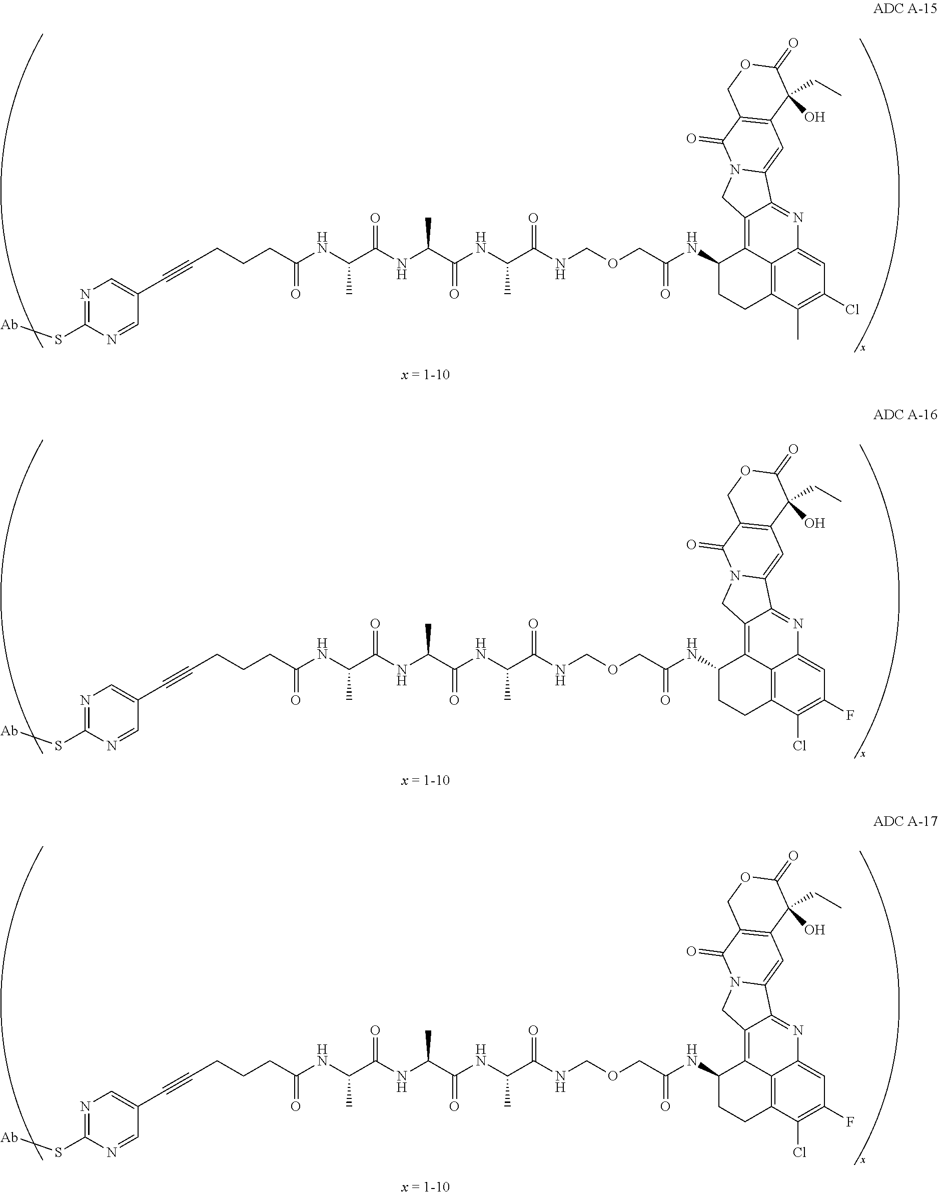

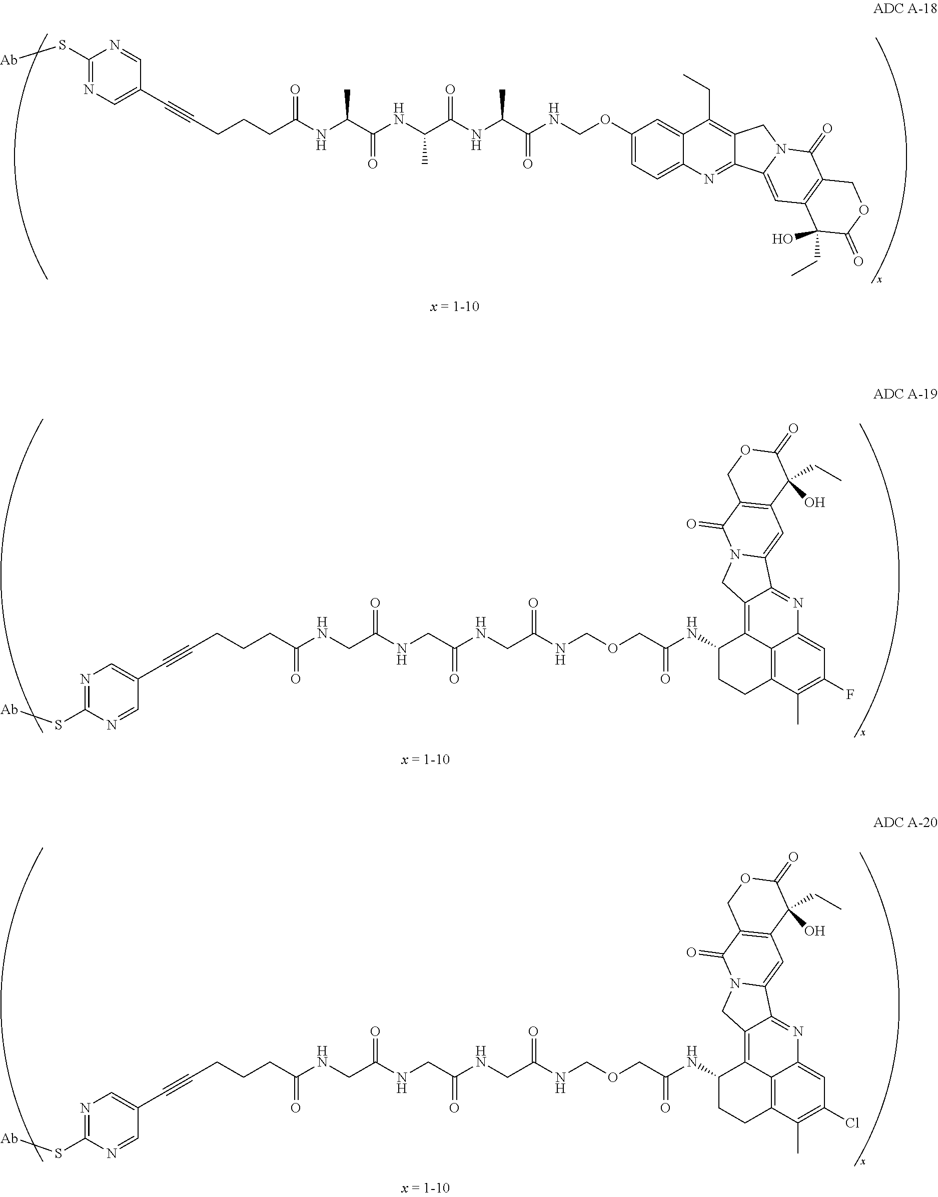

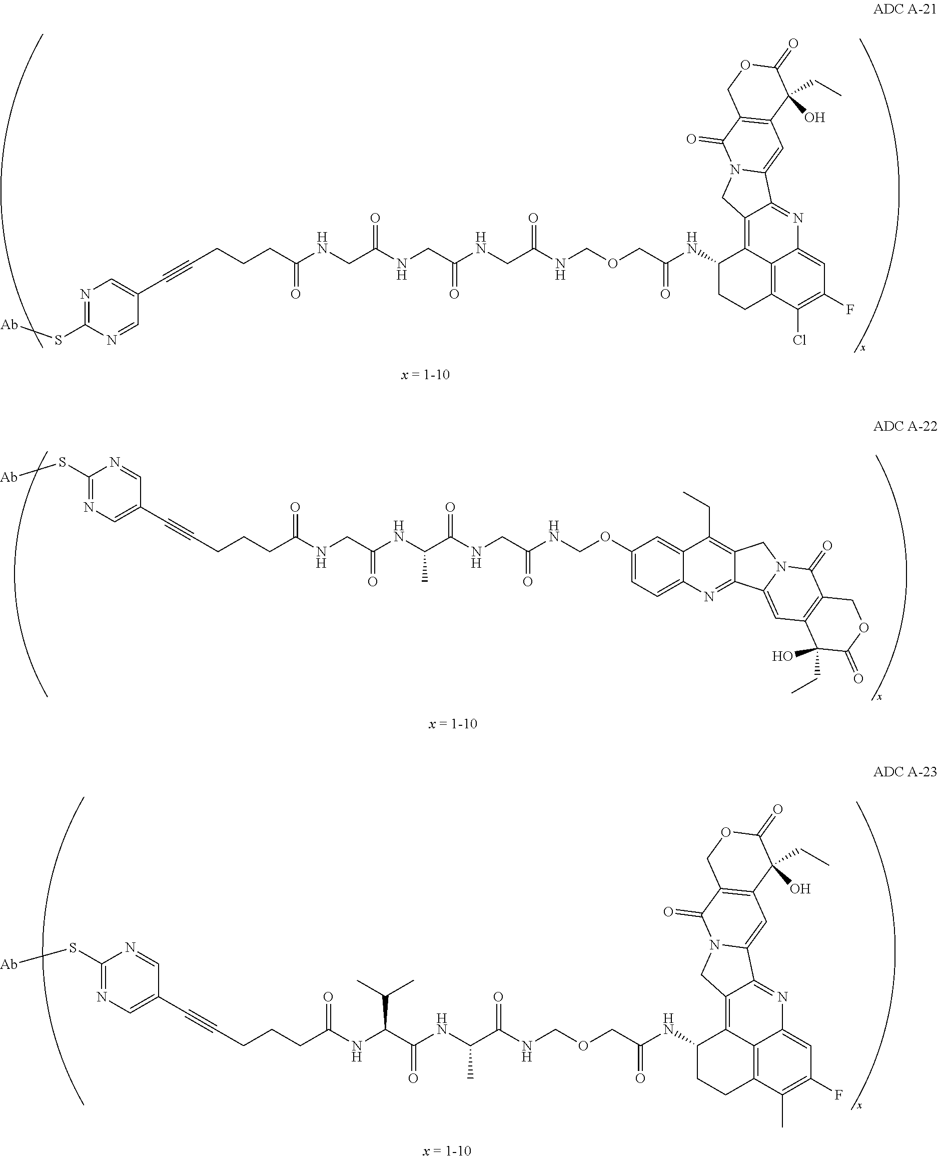

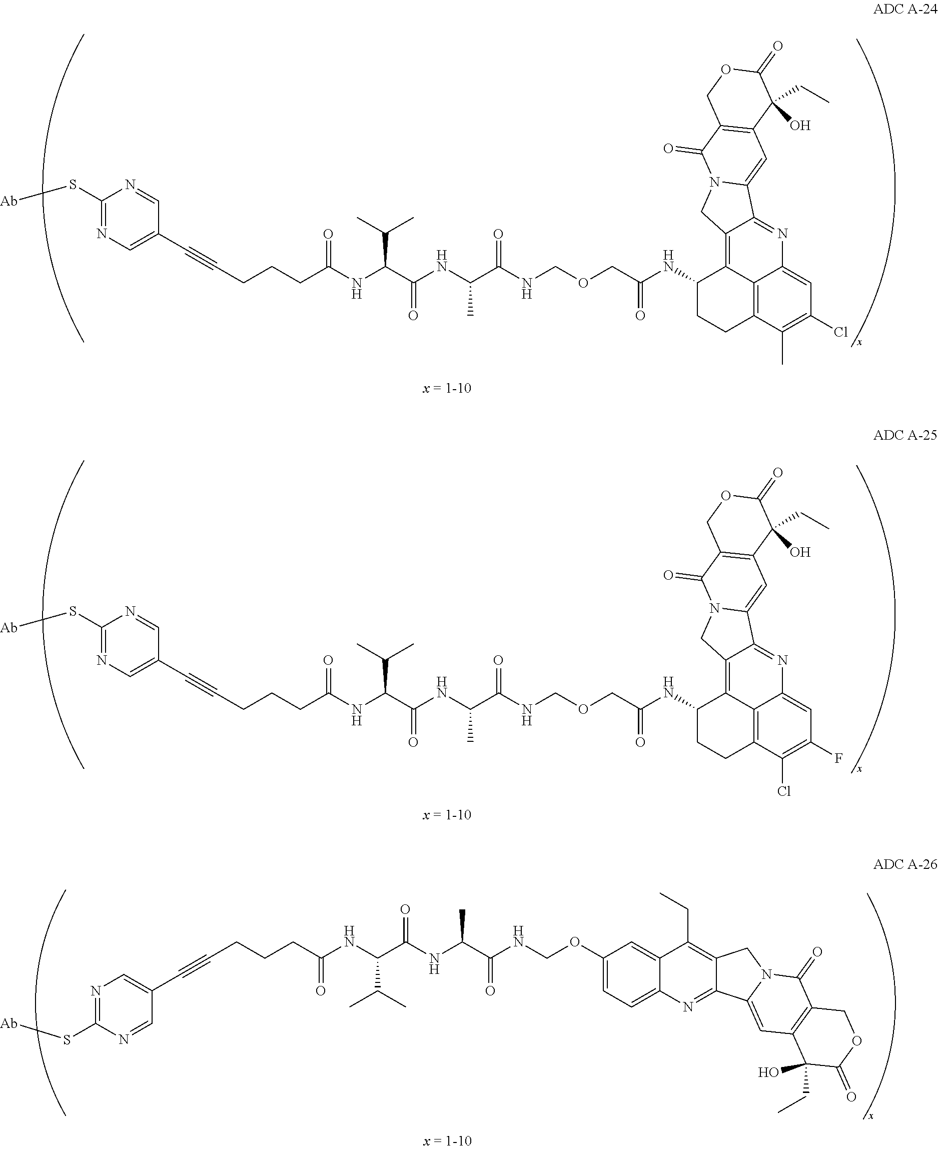

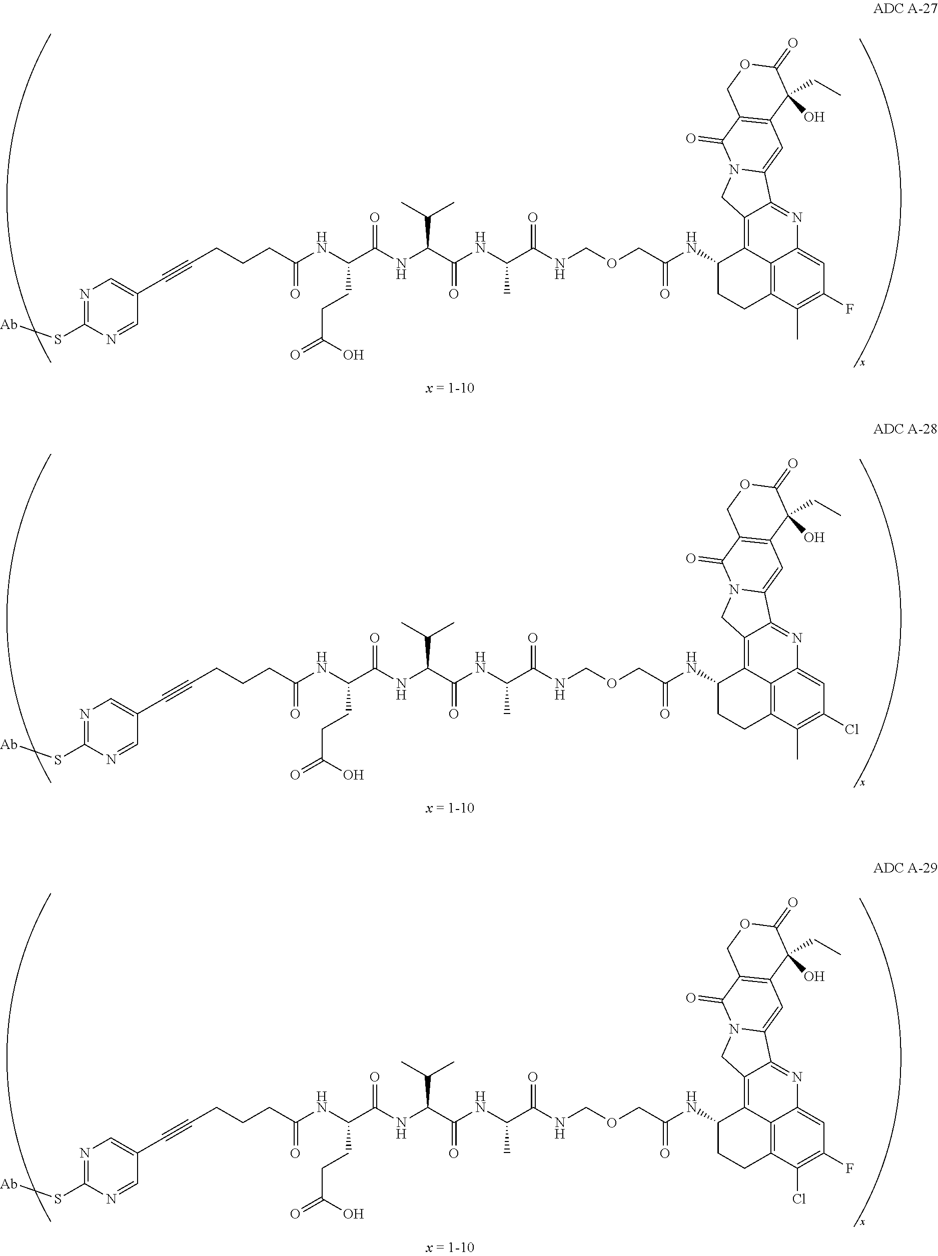

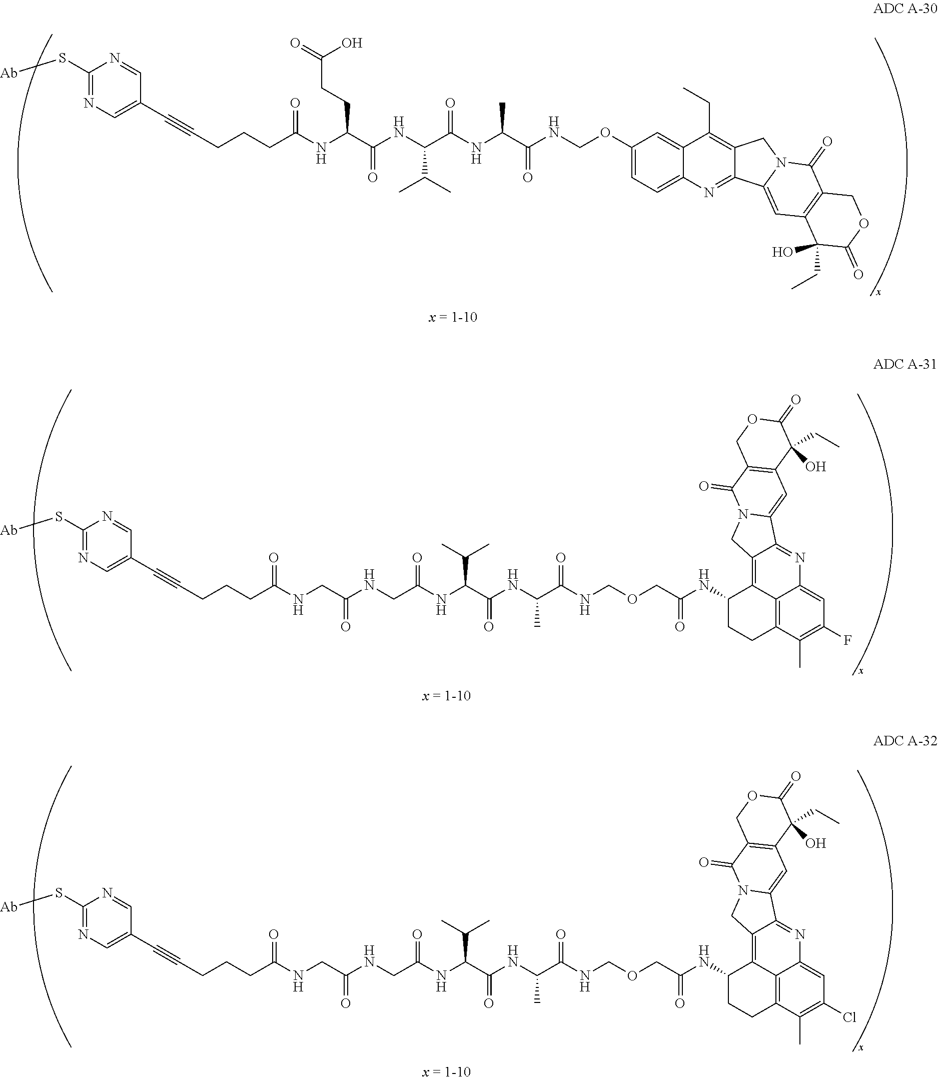

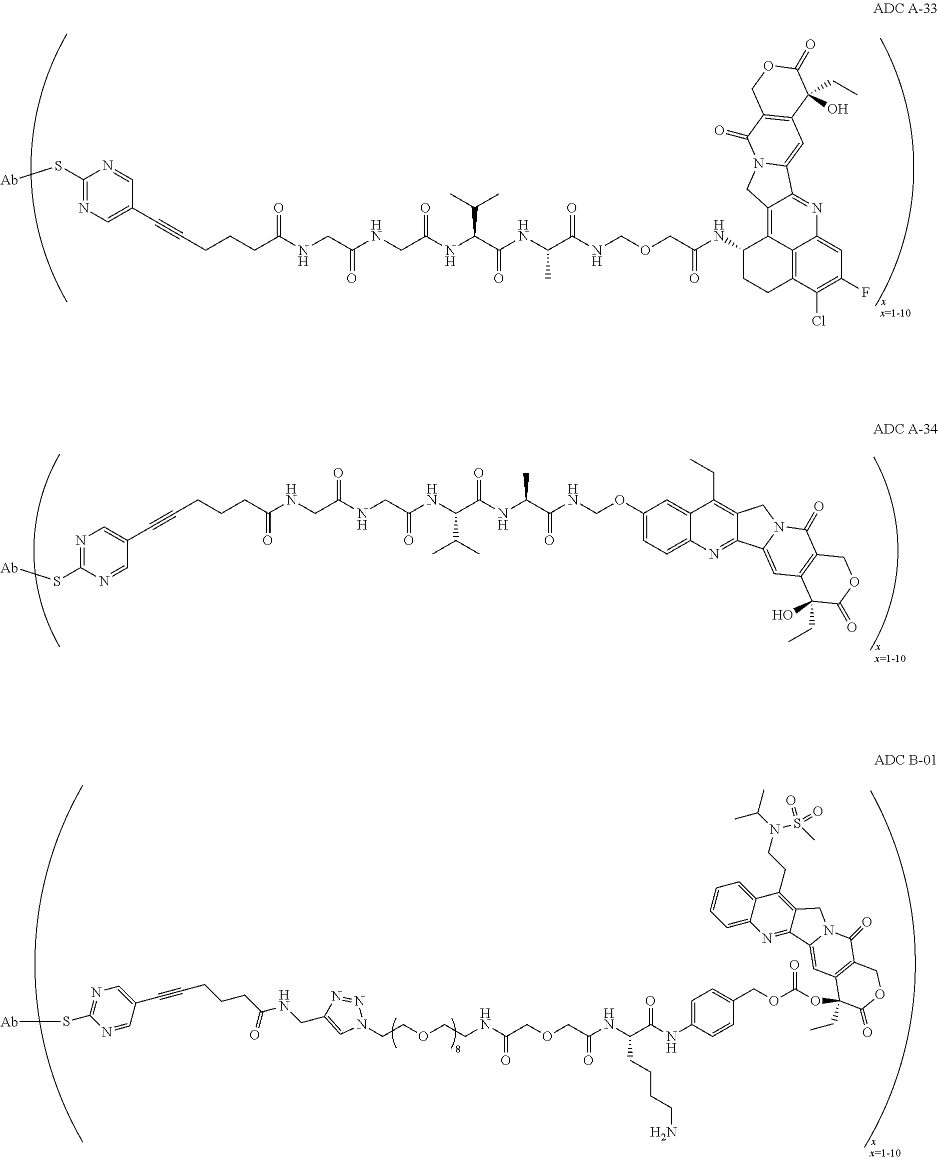

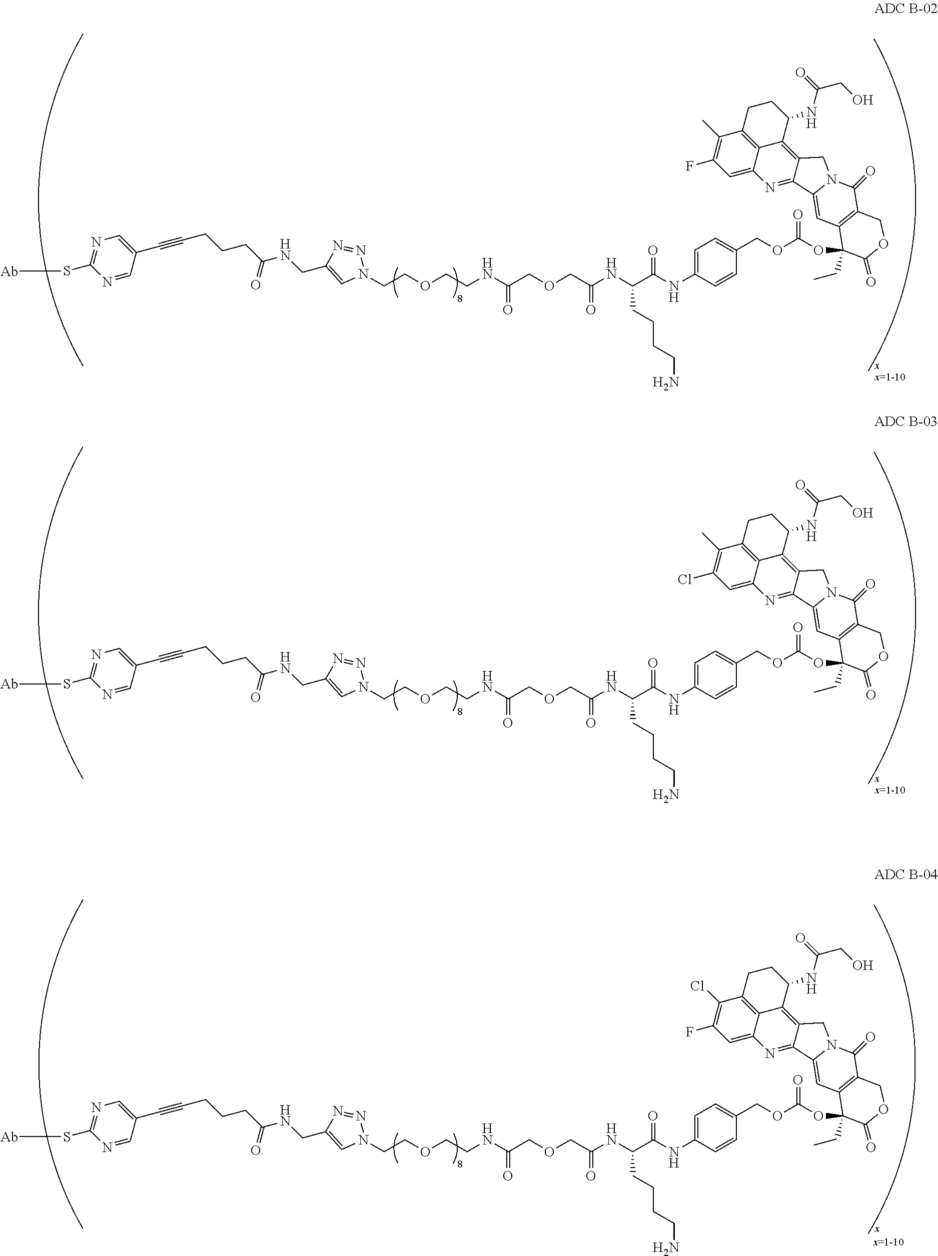

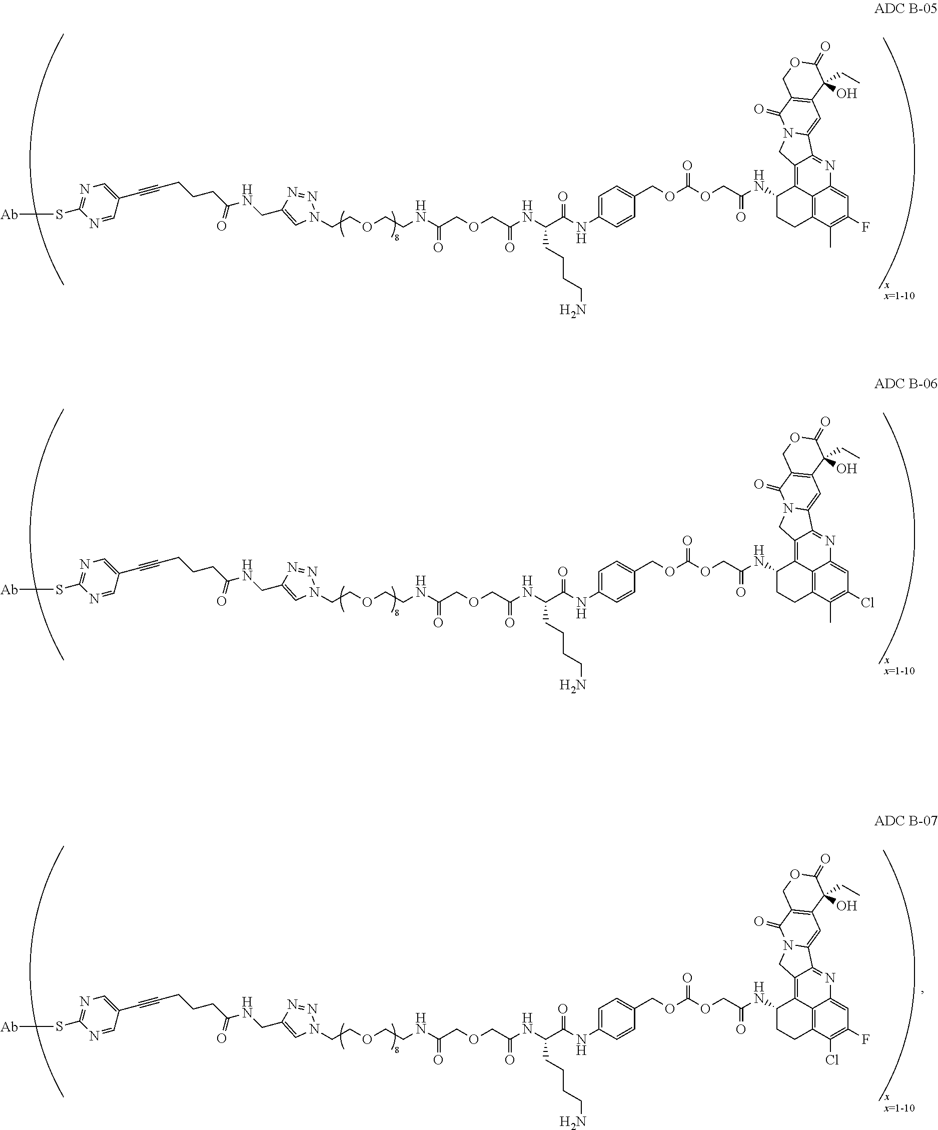

wherein Ab-(S— in each antibody-drug conjugate represents the antibody or the antigen-binding fragment thereof of any one above;
wherein

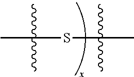 [SMILES: *S*]

represents a specific mode for connecting the sulfhydryl in the antibody or the antigen-binding fragment thereof with the linker.

In some embodiments, x′ in a conjugate shown in Ab′-(M-L-E-D)ₓ′ is any integer selected from 1-20, for example: 1, 2, 3, 4, 5, 6, 7, 8, 9, 10, 11, 12, 13, 14, 15, 16, 17, 18, 19, 20, 1-2, 1-3, 1-4, 1-5, 1-6, 1-7, 1-8, 1-9, 1-10, 1-16, 2-3, 2-4, 2-5, 2-6, 2-7, 2-8, 2-9, 2-10, 3-4, 3-5, 3- 6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10, in particular embodiments, 6-8.

In some embodiments, x′ is an integer.

In some embodiments, x′ in a conjugate shown in Ab′-(M-L-E-D)ₓ′ is about 8.

In some embodiments, the antibody-drug conjugate is selected from ADC A-05a, ADC A-14a, and ADC B-01a:

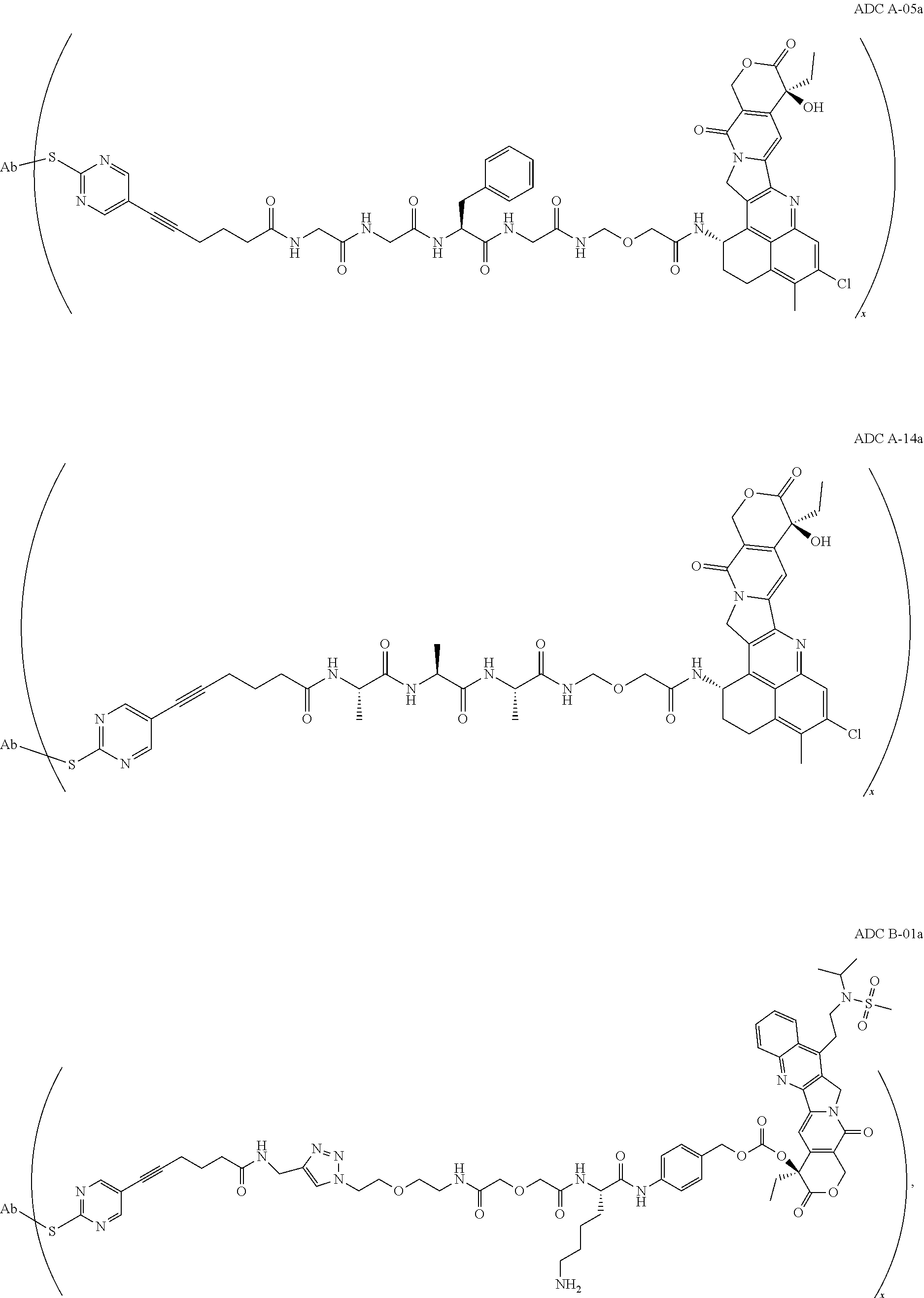

wherein Ab-(S— is an antibody or an antibody-binding fragment thereof of any one above, and —(S— represents a specific mode for connecting the sulfhydryl in the antibody or the antigen-binding fragment thereof with pyrimidyl in ADC A-05a, ADC A-14a, and ADC B-01a, and x is 1-16, 1-10, 1-8, 3-5, 7-9, 4-8, 4, or 8.

In some embodiments, an antibody-drug conjugate is selected from ADC A-05a, ADC A-14a, and ADC B-01a, and Ab-(S— comprises a VH comprising the amino acid sequence set forth in SEQ ID NO: 3 and a VL comprising the amino acid sequence set forth in SEQ ID NO: 4, or a VH comprising the amino acid sequence set forth in SEQ ID NO: 15 and a VL comprising the amino acid sequence set forth in SEQ ID NO: 16, wherein —(S— represents a specific mode for connecting the sulfhydryl in the antibody or the antigen-binding fragment thereof with pyrimidyl in ADC A-05a, ADC A-14a, and ADC B-01a, and x is 1-16, 1-10, 1-8, 3-5, 7-9, 4-8, 4, or 8. In some embodiments, —(S— represents a specific mode for connecting the sulfhydryl of a cysteine residue in the antibody or the antigen-binding fragment thereof with pyrimidyl in ADC A-05a, ADC A-14a, and ADC B-01a.

In some embodiments, an antibody-drug conjugate is selected from ADC A-05a, ADC A-14a, and ADC B-01a, and Ab-(S— comprises: a heavy chain comprising a VH amino acid sequence set forth in SEQ ID NO: 3 and a CH amino acid sequence set forth in SEQ ID NO: 121 and a light chain comprising a VL amino acid sequence set forth in SEQ ID NO: 4 and a CL amino acid sequence set forth in SEQ ID NO: 123, or a heavy chain comprising a VH amino acid sequence set forth in SEQ ID NO: 3 and a CH amino acid sequence set forth in SEQ ID NO: 122 and a light chain comprising a VL amino acid sequence set forth in SEQ ID NO: 4 and a CL amino acid sequence set forth in SEQ ID NO: 123, or a heavy chain comprising a VH amino acid sequence set forth in SEQ ID NO: 15 and the CH amino acid sequence set forth in SEQ ID NO:121 and a light chain including a VL amino acid sequence set forth in SEQ ID NO: 16 and a CL amino acid sequence set forth in SEQ ID NO: 123; wherein —(S— represents a specific mode for connecting the sulfhydryl in the antibody or the antigen-binding fragment thereof with pyrimidyl in ADC A-05a, ADC A-14a, and ADC B-01a, and x is 1-16, 1-10, 1-8, 3-5, 7-9, 4-8, 4, or 8.

In some embodiments, an antibody-drug conjugate is selected from ADC A-05b, ADC A-14b, and ADC B-01b:

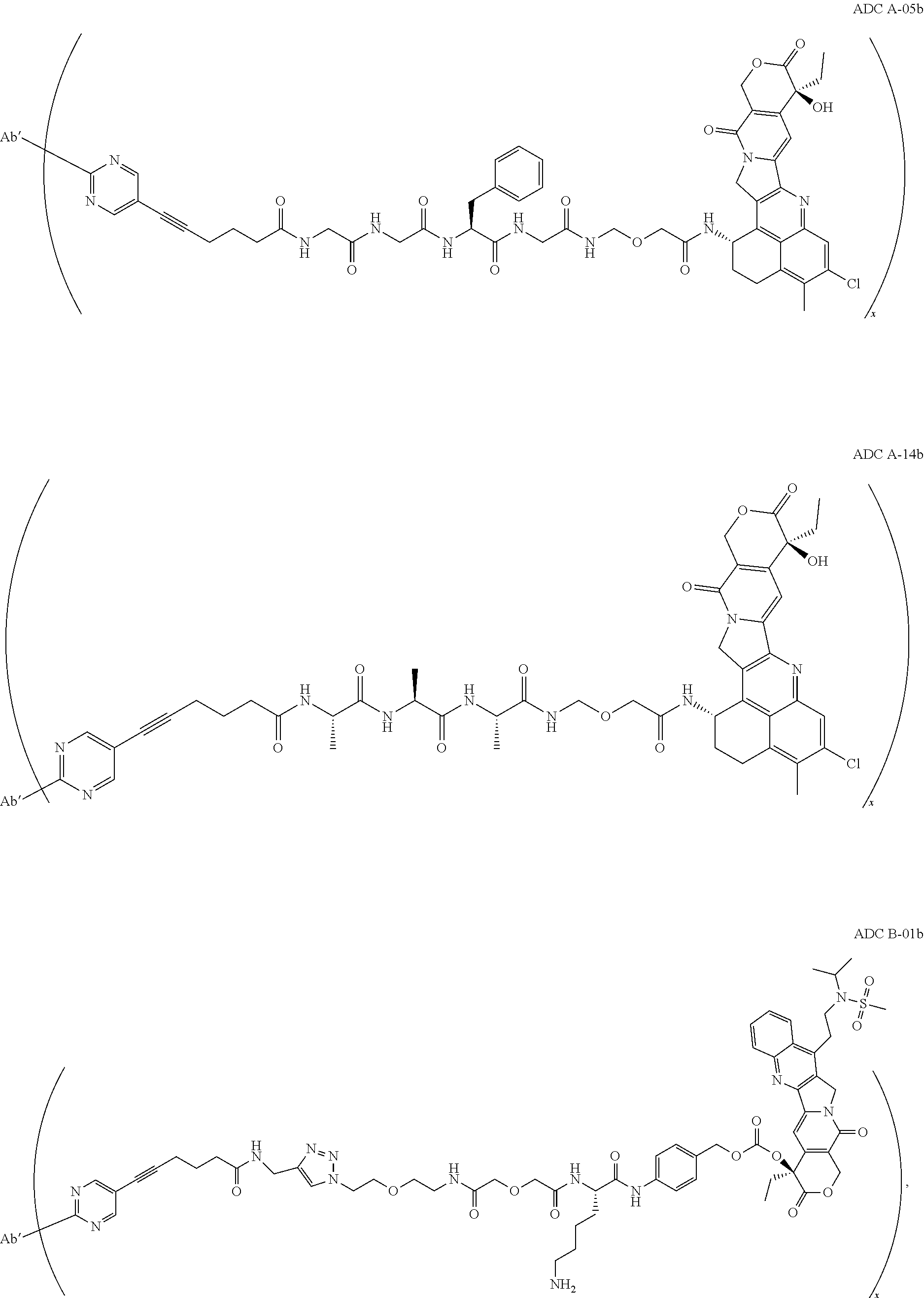

wherein Ab′ is an antibody or an antibody binding fragment thereof of any item above, and x is 1-16, 1-10, 1-8, 3-5,7-9,4-8,4, or 8.

In some embodiments, the antibody-drug conjugate is selected from ADC A-05b, ADC A-14b, and ADC B-01b, and Ab′ comprises a VH comprising the amino acid sequence set forth in SEQ ID NO: 3 and a VL comprising the amino acid sequence set forth in SEQ ID NO: 4, or a VH comprising the amino acid sequence set forth in SEQ ID NO: 15 and a VL comprising the amino acid sequence set forth in SEQ ID NO: 16, and x is 1-16, 1-10, 1-8, 3-5,7-9, 4-8, 4, or 8.

In some embodiments, an antibody-drug conjugate is selected from ADC A-05b, ADC A-14b, and ADC B-01b, and Ab′ comprises: a heavy chain comprising a VH amino acid sequence set forth in SEQ ID NO: 3 and a CH amino acid sequence set forth in SEQ ID NO: 121 and a light chain comprising a VL amino acid sequence set forth in SEQ ID NO: 4 and a CL amino acid sequence set forth in SEQ ID NO: 123, or a heavy chain comprising a VH amino acid sequence set forth in SEQ ID NO: 3 and a CH amino acid sequence set forth in SEQ ID NO: 122 and a light chain comprising a VL amino acid sequence set forth in SEQ ID NO: 4 and a CL amino acid sequence set forth in SEQ ID NO: 123, or a heavy chain comprising a VH amino acid sequence set forth in SEQ ID NO: 15 and the CH amino acid sequence set forth in SEQ ID NO: 121 and a light chain including a VL amino acid sequence set forth in SEQ ID NO: 16 and a CL amino acid sequence set forth in SEQ ID NO: 123; x is 1-16, 1-10, 1-8, 3-5, 7-9, 4-8, 4, or 8.

In some embodiments, Ab′ is attached by one or more sulfhydryl of a cysteine residue, one or more amino of a lysine residue, one or more hydroxy of a threonine residue or one or more hydroxy of a serine residue in an antibody, to form a conjugate.

In some embodiments, Ab′ is attached by one or more sulfhydryl of a cysteine residue in an antibody, to form a conjugate.

In some embodiments, Ab′ is attached by one or more amino of a lysine residue in an antibody, to form a conjugate.

In some embodiments, Ab′ is attached by one or more hydroxy of a threonine residue in an antibody, to form a conjugate.

In some embodiments, Ab′ is attached by one or more hydroxy of a serine residue in an antibody, to form a conjugate.

In some embodiments, Ab′ is attached by x sulfhydryl of a cysteine residue, x amino of a lysine residue, x hydroxy of a threonine residue or x hydroxy of a serine residue in an antibody, to form a conjugate.

In some embodiments, Ab′ is attached by x sulfhydryl of a cysteine residue in an antibody.

In some embodiments, Ab′ is attached by x amino of a lysine residue in an antibody.

In some embodiments, Ab′ is attached by x hydroxy of a threonine residue in an antibody.

In some embodiments, Ab′ is attached by x hydroxy of a serine residue in an antibody.

In some embodiments, an antibody in the antibody-drug conjugate is conjugated with 1-8 (for example, 1, 2, 3, 4, 5, 6, 7, 8; in particular embodiments, 5-8) following structures:

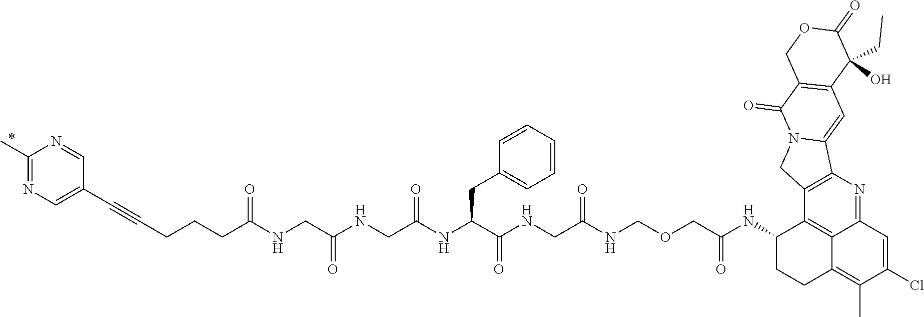

wherein * is an attachment point for conjugating sulfhydryl of cysteine of an antibody.

In some embodiments, an antibody in the antibody-drug conjugate is conjugated with 1-8 (for example, 1, 2, 3, 4, 5, 6, 7, 8; in particular embodiments, 5-8) following structures:

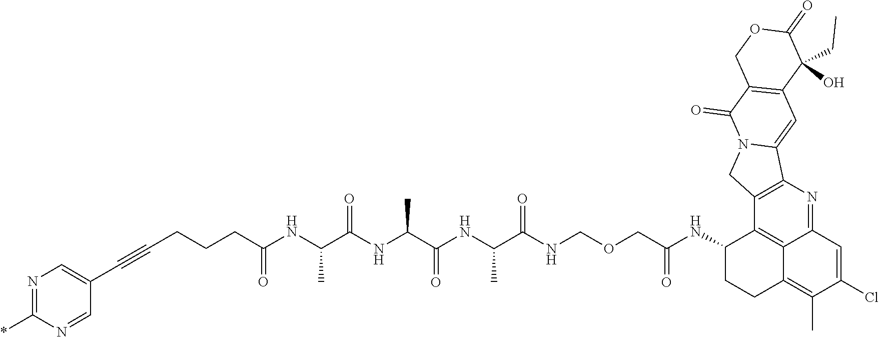 [SMILES: *c1ncc(C#CCCCC(=O)N[C@@H](C)C(=O)N[C@@H](C)C(=O)N[C@@H](C)C(=O)NCOCC(=O)N[C@H]2CCc3c(C)c(Cl)cc4nc5c(c2c34)Cn2c-5cc3c(c2=O)COC(=O)[C@]3(O)CC)cn1]

wherein * is an attachment point for conjugating sulfhydryl of cysteine of an antibody.

In some embodiments, an antibody in the antibody-drug conjugate is conjugated with 1-8 (for example, 1, 2, 3, 4, 5, 6, 7, 8; in particular embodiments, 5-8) following structures:

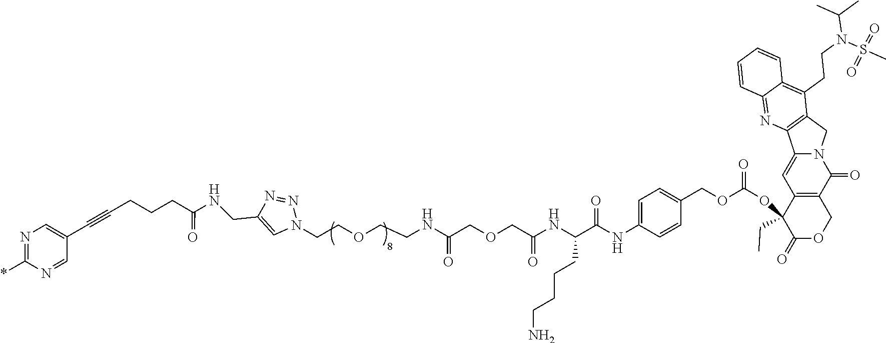

wherein * is an attachment point for conjugating sulfhydryl of cysteine of an antibody.

In some embodiments, an antibody-drug conjugate described in the present disclosure is optionally substituted with one or more suitable substituents.

In the above formula I of the present disclosure, the groups of all embodiments can be appropriately selected for any combination to obtain different general formula ranges or specific schemes. These ranges and schemes belong to the present disclosure. the present disclosure encompasses antibody-drug conjugates obtained by any combination of various embodiments.

### Composition

In another aspect, the present disclosure provides a composition of the antibody-drug conjugate (ADC) described herein. Such a composition may comprise a plurality of ADCs described herein, wherein each ADC comprises the drug-linker described herein, wherein x is independently 1, 2, 3, 4, 5, 6, 7, 8, 9, or 10. In other words, each antibody molecule in the composition can be conjugated with 1, 2, 3, 4, 5, 6, 7, 8, 9, or 10 drug-linkers. Therefore, the composition is characterized in that a “drug-antibody” ratio (DAR) is in the range of about 1 to about 10. Methods for determining DAR are well known to technicians, including a method using reverse phase chromatography or HPLC-MS.

In some embodiments, a DAR (drug-antibody conjugation ratio) value of the ADC composition is 1-10, for example, 1, 2, 3, 4, 5, 6, 7, 8, 9, 10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4- 9, 4-10, 5-6, 5-7, 5-8, 5-9, 5-10, 6-7, 6-8, 6-9, 6-10, 7-8, 7-9, 7-10, 8-9, 8-10 or 9-10, in particular embodiments, 1-8, in particular embodiments, 3-9, for example, 3.0-3.5, 3.0-4.0, 3.0-4.5, 3.0-5.0, 3.0-5.5, 3.0-6.0, 3.5-4.0, 3.5-4.5, 3.5-5.0, 3.5-5.5, 3.5-6.0, 3.5-6.5, 3.5-7.0, 3.5-7.5, 3.5-8.0, 4.0-4.5, 4.0-5.0, 4.0-5.5, 4.0-6.0, 4.0-6.5, 4.0-7.0, 4.0-7.5, 4.0-8.0, 4.5-5.0, 4.5-5.5, 4.5-6.0, 4.5-6.5, 4.5-7.0, 4.5-7.5, 4.5-8.0, 5.0-5.5, 5.0-6.0, 5.0-6.5, 5.0-7.0, 5.0-7.5, 5.0-8.0, 5.5-6.0, 5.5-6.5, 5.5-7.0, 5.5-7.5, 5.5-8.0, 6.0-6.5, 6.0-7.0, 6.0-7.5, 6.0-8.5, 6.5-7.0, 6.5-7.5, 6.5-8.5, 7.0-7.5, 7.0-9.0, or 7.5-9.0.

In some embodiments, a DAR of the ADC composition described herein is about 6.0 to 10.0, for example, about 6.0, about 6.01, about 6.02, about 6.03, about 6.04, about 6.05, about 6.06, about 6.07, about 6.08, about 6.09, about 6.1, about 6.11, about 6.12, about 6.13, about 6.14, about 6.15, about 6.16, about 6.17, about 6.18, about 6.19, about 6.2, about 6.21, about 6.22, about 6.23, about 6.24, about 6.25, about 6.26, about 6.27, about 6.28, about 6.29, about 6.3, about 6.31, about 6.32, about 6.33, about 6.34, about 6.35, about 6.36, about 6.37, about 6.38, about 6.39, about 6.4, about 6.41, about 6.42, about 6.43, about 6.44, about 6.45, about 6.46, about 6.47, about 6.48, about 6.49, about 6.5, about 6.51, about 6.52, about 6.53, about 6.54, about 6.55, about 6.56, about 6.57, about 6.58, about 6.59, about 6.6, about 6.61, about 6.62, about 6.63, about 6.64, about 6.65, about 6.66, about 6.67, about 6.68, about 6.69, about 6.7, about 6.71, about 6.72, about 6.73, about 6.74, about 6.75, about 6.76, about 6.77, about 6.78, about 6.79, about 6.8, about 6.81, about 6.82, about 6.83, about 6.84, about 6.85, about 6.86, about 6.87, about 6.88, about 6.89, about 6.9, about 6.91, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, about 7.1, about 7.11, about 7.12, about 7.13, about 7.14, about 7.15, about 7.16, about 7.17, about 7.18, about 7.19, about 7.2, about 7.21, about 7.22, about 7.23, about 7.24, about 7.25, about 7.26, about 7.27, about 7.28, about 7.29, about 7.3, about 7.31, about 7.32, about 7.33, about 7.34, about 7.35, about 7.36, about 7.37, about 7.38, about 7.39, about 7.4, about 7.41, about 7.42, about 7.43, about 7.44, about 7.45, about 7.46, about 7.47, about 7.48, about 7.49, about 7.5, about 7.51, about 7.52, about 7.53, about 7.54, about 7.55, about 7.56, about 7.57, about 7.58, about 7.59, about 7.6, about 7.61, about 7.62, about 7.63, about 7.64, about 7.65, about 7.66, about 7.67, about 7.68, about 7.69, about 7.7, about 7.71, about 7.72, about 7.73, about 7.74, about 7.75, about 7.76, about 7.77, about 7.78, about 7.79, about 7.8, about 7.81, about 7.82, about 7.83, about 7.84, about 7.85, about 7.86, about 7.87, about 7.88, about 7.89, about 7.9, about 7.91, about 7.92, about 7.93, about 7.94, about 7.95, about 7.96, about 7.97, about 7.98, about 7.99, about 8.0, about 8.01, about 8.02, about 8.03, about 8.04, about 8.05, about 8.06, about 8.07, about 8.08, about 8.09, about 8.1, about 8.11, about 8.12, about 8.13, about 8.14, about 8.15, about 8.16, about 8.17, about 8.18, about 8.19, about 8.2, about 8.21, about 8.22, about 8.23, about 8.24, about 8.25, about 8.26, about 8.27, about 8.28, about 8.29, about 8.3, about 8.31, about 8.32, about 8.33, about 8.34, about 8.35, about 8.36, about 8.37, about 8.38, about 8.39, about 8.4, about 8.41, about 8.42, about 8.43, about 8.44, about 8.45, about 8.46, about 8.47, about 8.48, about 8.49, about 8.5, about 8.51, about 8.52, about 8.53, about 8.54, about 8.55, about 8.56, about 8.57, about 8.58, about 8.59, about 8.6, about 8.61, about 8.62, about 8.63, about 8.64, about 8.65, about 8.66, about 8.67, about 8.68, about 8.69, about 8.7, about 8.71, about 8.72, about 8.73, about 8.74, about 8.75, about 8.76, about 8.77, about 8.78, about 8.79, about 8.8, about 8.81, about 8.82, about 8.83, about 8.84, about 8.85, about 8.86, about 8.87, about 8.88, about 8.89, about 8.9, about 8.91, about 8.92, about 8.93, about 8.94, about 8.95, about 8.96, about 8.97, about 8.98, about 8.99, about 9.0, about 6.0-8.0, about 6.0-8.5, about 6.0-9.0, about 6.0-9.5, about 6.0-10.0, about 6.5-8.0, about 6.5-8.5, about 6.5-9.0, about 6.5-9.5, about 6.5-10.0, about 7.0-8.0, about 7.0-8.5, about 7.0-9.0, about 7.0-9.5, about 7.0-10.0, about 7.5-8.0, about 7.5-8.5, about 7.5-9.0, about 7.5-9.5, about 7.5-10.0, about 8.0-8.5, about 8.0-9.0, about 8.0-9.5, about 8.0-10.0.

### Methods of Preparation

Those skilled in the art will understand that the antibody-drug conjugate described in the present disclosure can be prepared modularly. For example, a “drug-linker” in a pre-conjugation form (which can be understood as M′-L-E-D, wherein M′ is a structural form before M is covalently linked to an antibody or an antigen-binding fragment thereof) is first obtained, and then it is covalently linked to the antibody or the antigen-binding fragment thereof to obtain the antibody-drug conjugate described in the present disclosure. The “drug-linker” in the pre-conjugation form can be a “drug-linker” in a free form. Accordingly, M′ in the “drug-linker” in the pre-conjugation form is attached to one or more sulfhydryl (—SH), amino (—NH₂) or carboxyl (—COOH) on the antibody or the antigen-binding fragment thereof by a substitution reaction (for example, removing structures such as —SO₂Me or —Br thereon) or by an addition reaction, etc.

In another aspect, the present disclosure provides a drug-linker having a structure shown in the formula M′-L-E-D, wherein:
M′ is

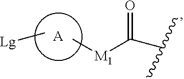

Lg is a leaving group (for example, halogen (for example, F, Cl, Br, I), halogenated C₁₋₆alkyl, C₁₋₆alkylsulfonyl, halogenated C₁₋₆alkylsulfonyl, halogenated sulfonyl, C₁₋₆alkylsulfonate, halogenated C₁₋₆alkylsulfonate, C₁₋₆alkylsulfinate, C₁₋₆alkylsulfoxide, halogenated phenoxy, hydroxy, sulfhydryl, amino, nitro, azido, cyano, alkenyl, alkynyl, alkynyl-containing structural fragments or

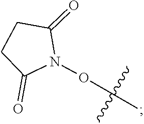

the halogenated C₁₋₆alkyl, C₁₋₆alkylsulfonyl, halogenated C₁₋₆alkylsulfonyl, halogenated sulfonyl, C₁₋₆alkylsulfonate, halogenated C₁₋₆alkylsulfonate, C₁₋₆alkylsulfinate, C₁₋₆alkylsulfoxide, halogenated phenoxy, alkenyl, alkynyl and alkynyl-containing structural fragments are optionally substituted with one or more suitable substituents) for a nucleophilic substitution reaction, or hydroxy (—OH), sulfhydryl (—SH) or amino (—NH₂); or, Lg forms an unsaturated double bond with an adjacent atom on ring A; ring A is a 5-6-membered aliphatic heterocyclic ring or a 5-20-membered aromatic ring system, wherein the aliphatic heterocyclic ring and the aromatic ring system are optionally substituted with one or more groups selected from oxo (=O), halogen, cyano, amino, carboxyl, sulfhydryl, and C₁₋₆ alkyl; M₁ is selected from a single bond and C₁₋₂₀alkylene, C₂₋₂₀alkenylene, or C₂₋₂₀alkynylene; and
L, E, and D structures are defined in any one of the above antibody-drug conjugates.

In some embodiments, M′ is

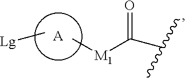

Lg is methylsulfonyl, or Lg forms a carbon-carbon double bond with an adjacent atom on ring A; ring A is a 5-membered aliphatic heterocylic ring, a 6-membered heteroaromatic ring, or a polycyclic ring formed by connecting more than one 6-membered heteroaromatic ring with a phenyl ring via a single bond, and the aliphatic heterocyclic ring is optionally substituted with one or more groups selected from oxo (═O), halogen, and C₁₋₄alkyl; and M₁ is selected from a single bond and C₃₋₁₀alkylene, C₃₋₁₀alkenylene, or C₃₋₁₀alkynylene.

In some embodiments, M′ is

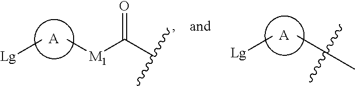

is selected from

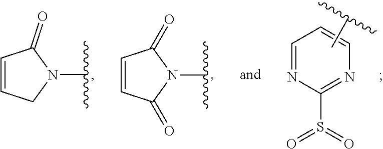

and M₁ is selected from a single bond and C₅₋₈alkylene, C₅₋₈ alkenylene, and C₅₋₈alkynylene.

In some embodiments, M′ is selected from

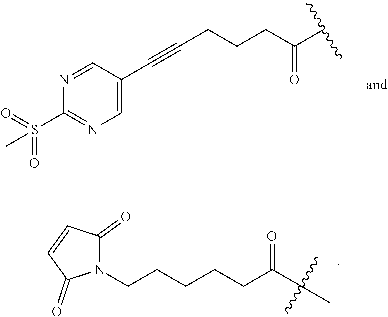 [SMILES: *C(=O)CCCC#Cc1cnc(S(C)(=O)=O)nc1 ; "and" ; *C(=O)CCCCCN1C(=O)C=CC1=O ; "."]

In some embodiments, the “drug-linker” in the free form is selected from A-01 to A-34 and B-01 to B-07 as shown below:

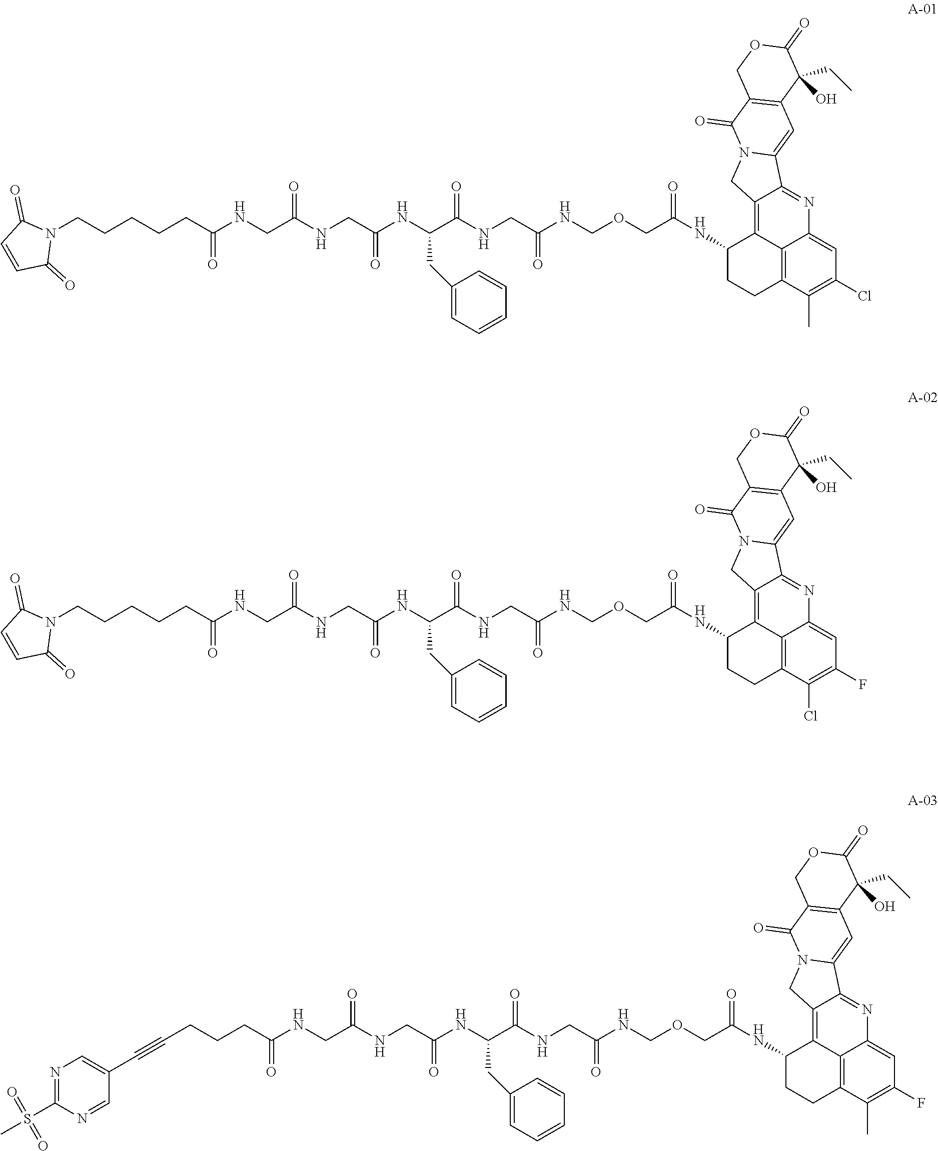 [SMILES: CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(Cl)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3]

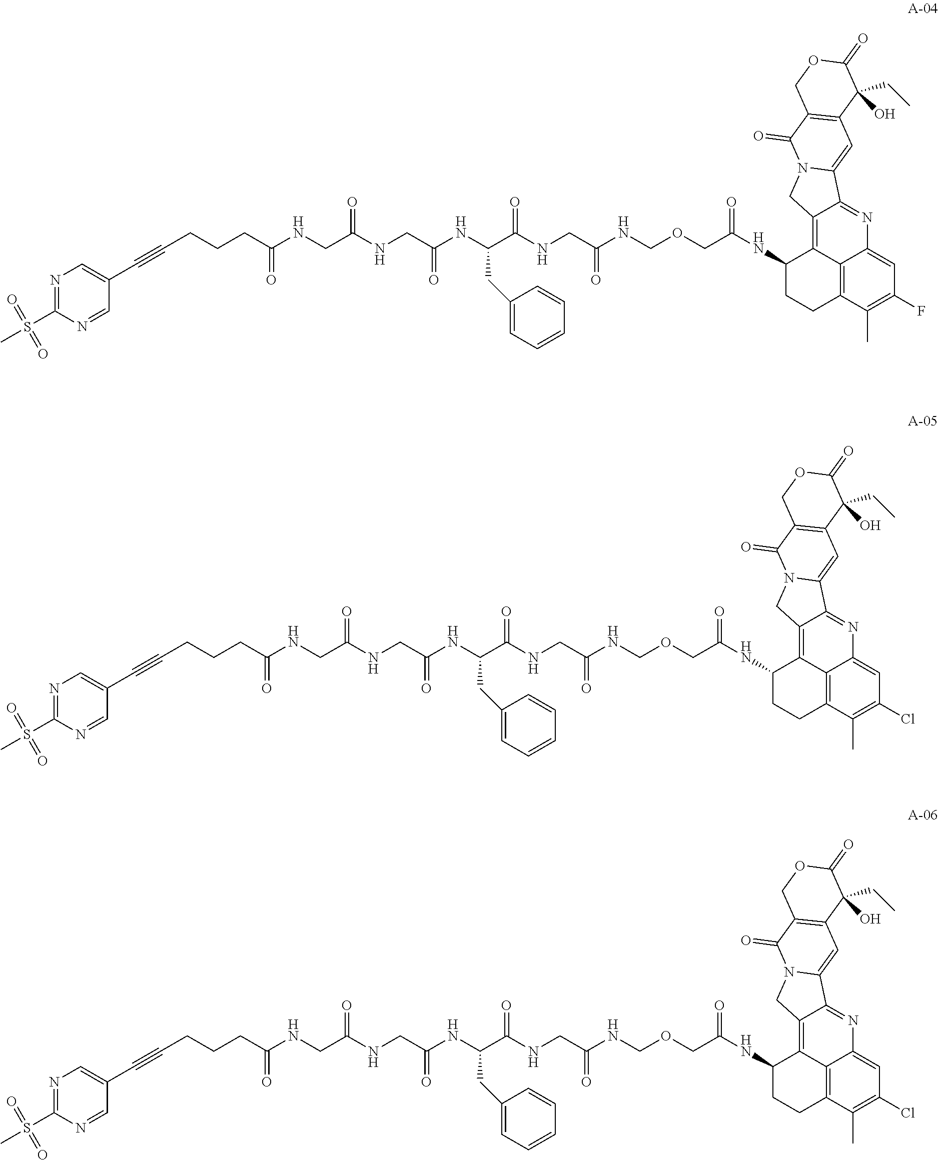 [SMILES: CC[C@@]1(O)C(=O)OCc2c1cc1n(c2=O)Cc2c-1nc1cc(F)c(C)c3c1c2[C@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; CC[C@@]1(O)C(=O)OCc2c1cc1n(c2=O)Cc2c-1nc1cc(Cl)c(C)c3c1c2[C@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3]

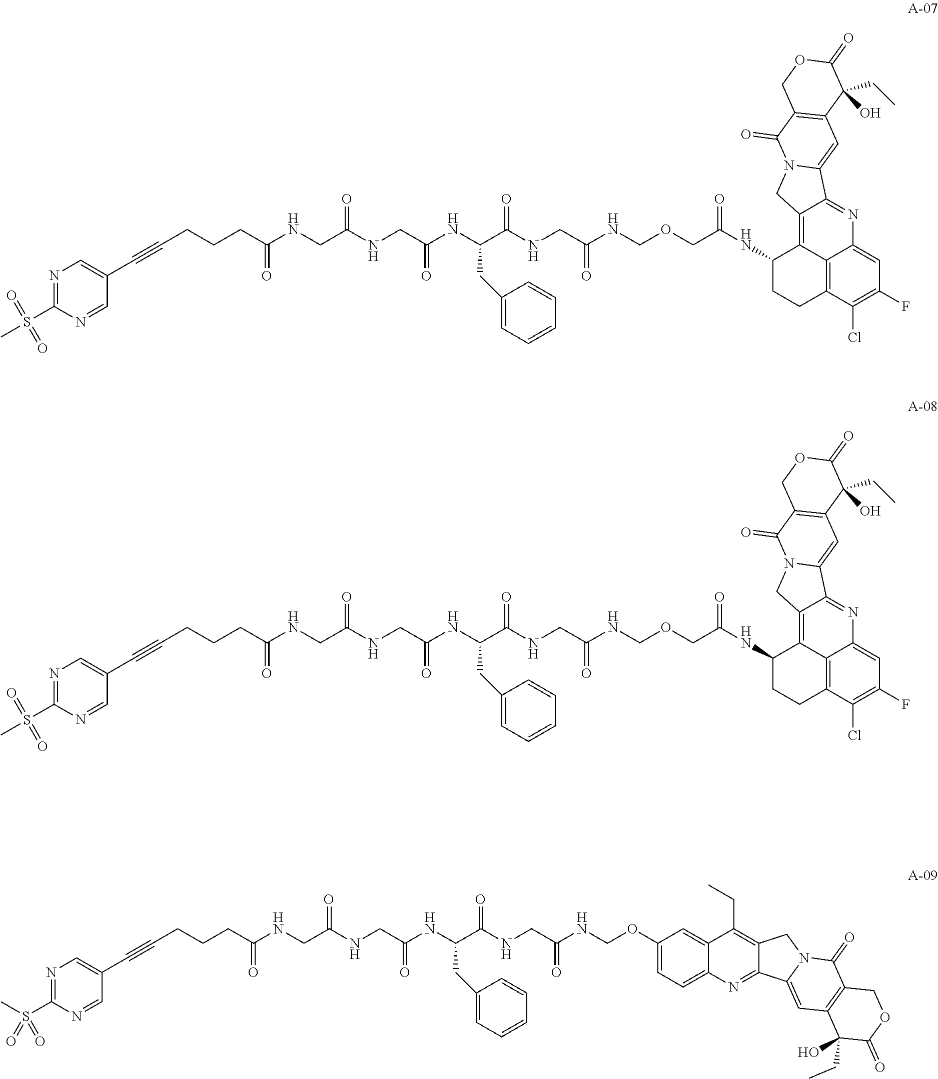 [SMILES: CC[C@@]1(O)C(=O)OCc2c1cc1n(c2=O)Cc2c-1nc1cc(F)c(Cl)c3c1c2[C@@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; CC[C@@]1(O)C(=O)OCc2c1cc1n(c2=O)Cc2c-1nc1cc(F)c(Cl)c3c1c2[C@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; CCc1c2c(nc3ccc(OCNC(=O)CNC(=O)[C@H](Cc4ccccc4)NC(=O)CNC(=O)CNC(=O)CCCC#Cc4cnc(S(C)(=O)=O)nc4)cc13)-c1cc3c(c(=O)n1C2)COC(=O)[C@]3(O)CC]

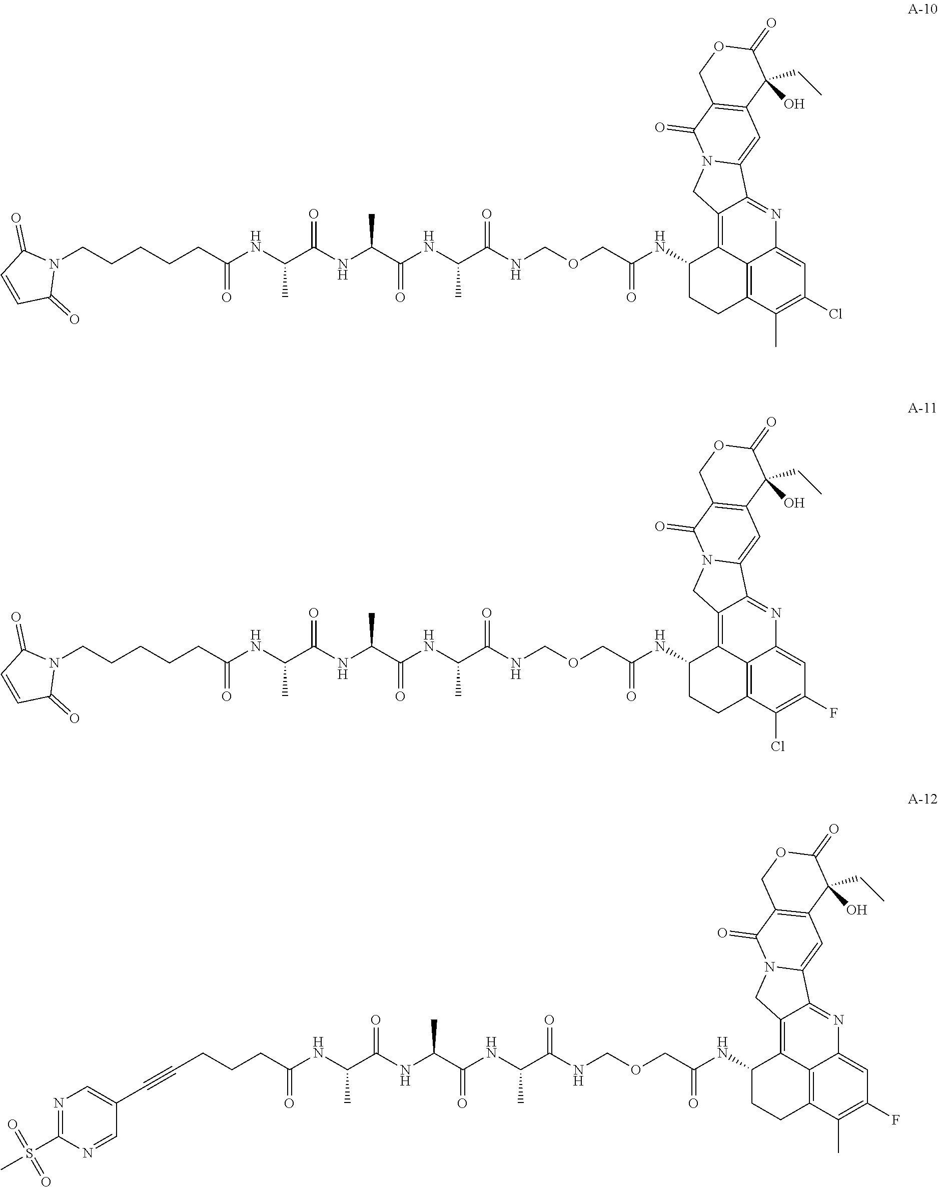 [SMILES: CC[C@@]1(O)C(=O)OCc2c1cc1n(c2=O)Cc2c-1nc1cc(Cl)c(C)c3c1c2[C@@H](NC(=O)COCNC(=O)[C@H](C)NC(=O)[C@H](C)NC(=O)[C@H](C)NC(=O)CCCCCN1C(=O)C=CC1=O)CC3 ; CC[C@@]1(O)C(=O)OCc2c1cc1n(c2=O)Cc2c-1nc1cc(F)c(Cl)c3c1c2[C@@H](NC(=O)COCNC(=O)[C@H](C)NC(=O)[C@H](C)NC(=O)[C@H](C)NC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@H](C)NC(=O)[C@H](C)NC(=O)CCCC#Cc1cnc(S(C)(=O)=O)nc1)CC3]

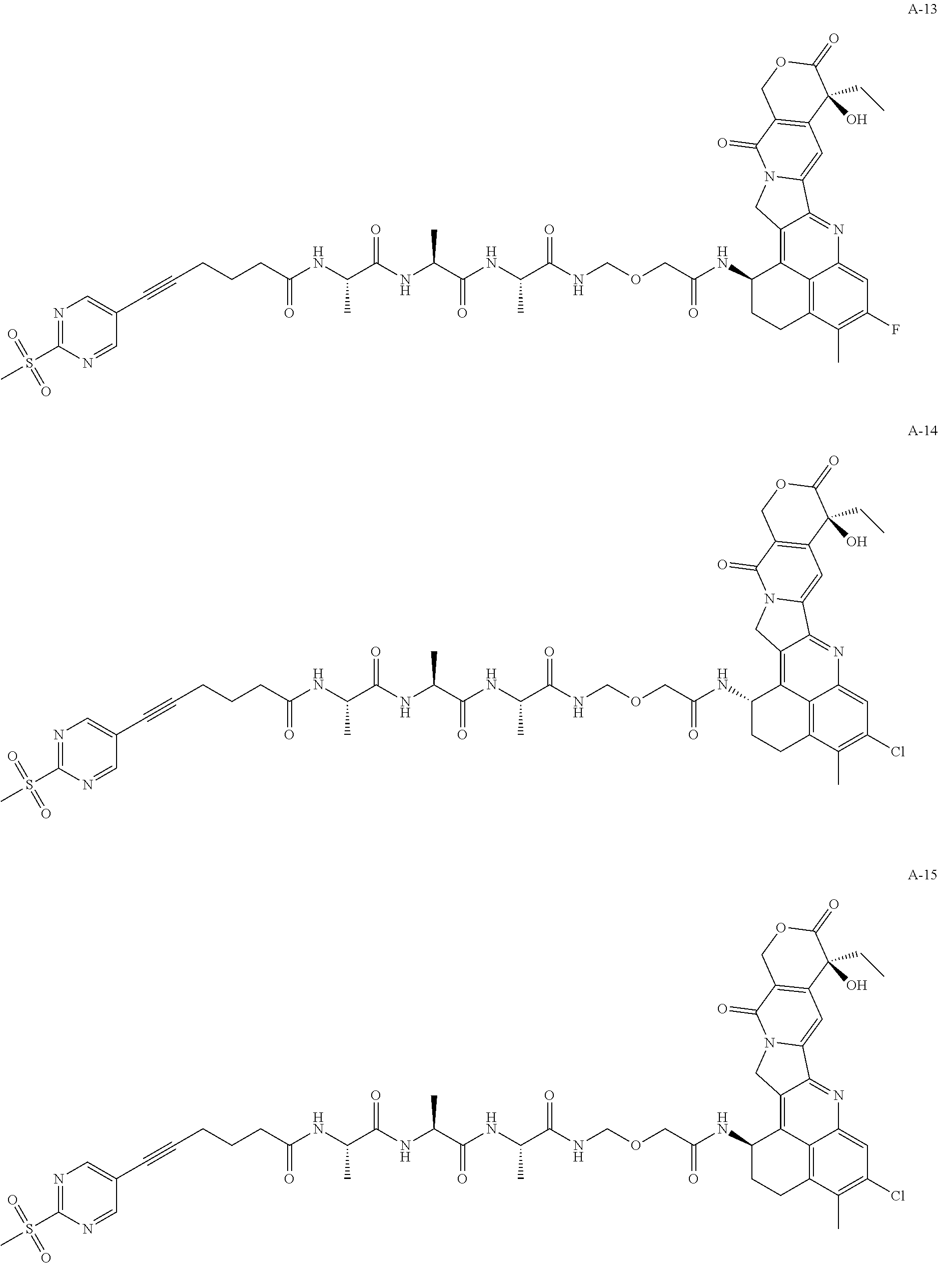 [SMILES: CC[C@@]1(O)C(=O)OCc2c1cc1n(c2=O)Cc2c-1nc1cc(F)c(C)c3c1c2[C@H](NC(=O)COCNC(=O)[C@H](C)NC(=O)[C@H](C)NC(=O)[C@H](C)NC(=O)CCCC#Cc1cnc(S(C)(=O)=O)nc1)CC3 ; CC[C@@]1(O)C(=O)OCc2c1cc1n(c2=O)Cc2c-1nc1cc(Cl)c(C)c3c1c2[C@@H](NC(=O)COCNC(=O)[C@H](C)NC(=O)[C@H](C)NC(=O)[C@H](C)NC(=O)CCCC#Cc1cnc(S(C)(=O)=O)nc1)CC3 ; CC[C@@]1(O)C(=O)OCc2c1cc1n(c2=O)Cc2c-1nc1cc(Cl)c(C)c3c1c2[C@H](NC(=O)COCNC(=O)[C@H](C)NC(=O)[C@H](C)NC(=O)[C@H](C)NC(=O)CCCC#Cc1cnc(S(C)(=O)=O)nc1)CC3]

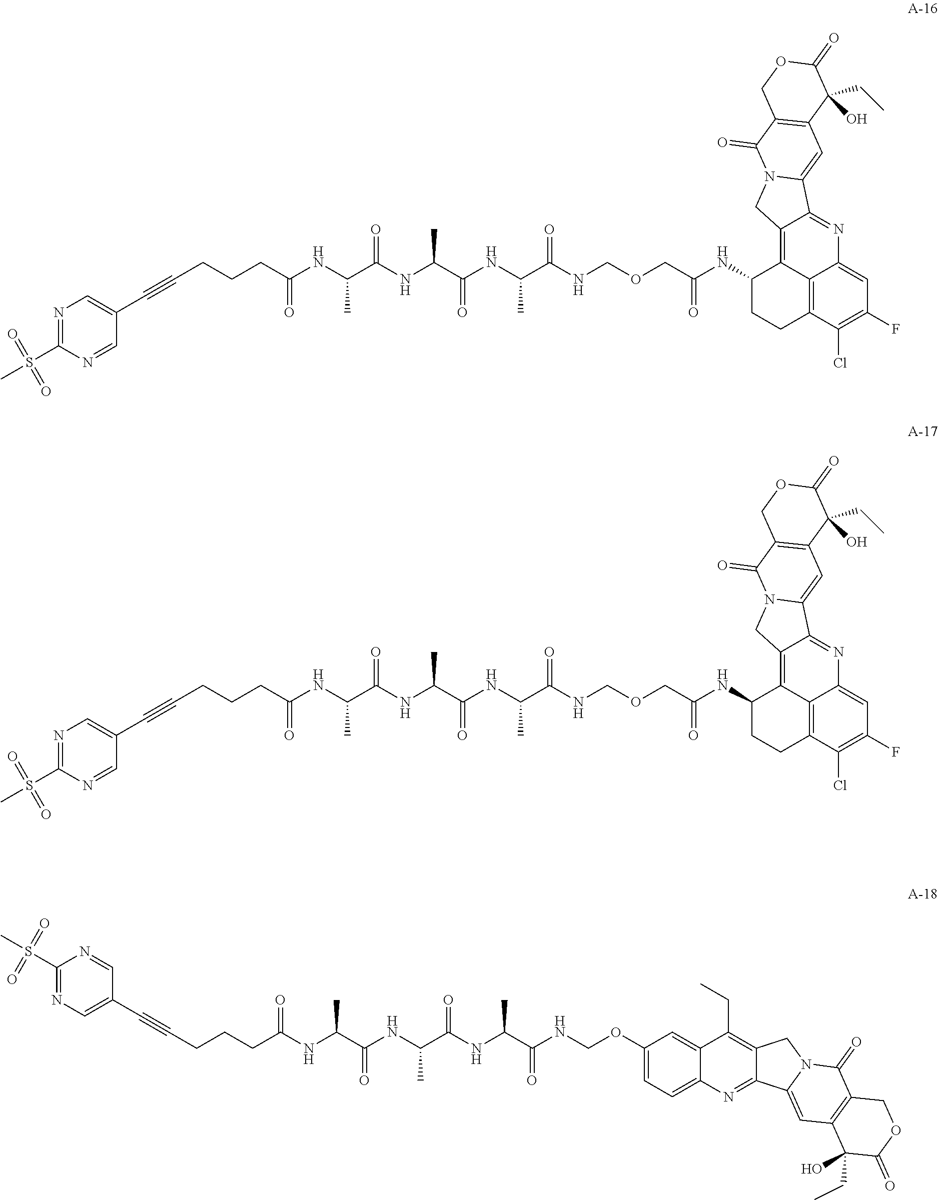 [SMILES: CC[C@@]1(O)C(=O)OCc2c1cc1n(c2=O)Cc2c-1nc1cc(F)c(Cl)c3c1c2[C@@H](NC(=O)COCNC(=O)[C@H](C)NC(=O)[C@H](C)NC(=O)[C@H](C)NC(=O)CCCC#Cc1cnc(S(C)(=O)=O)nc1)CC3 ; CC[C@@]1(O)C(=O)OCc2c1cc1n(c2=O)Cc2c-1nc1cc(F)c(Cl)c3c1c2[C@H](NC(=O)COCNC(=O)[C@H](C)NC(=O)[C@H](C)NC(=O)[C@H](C)NC(=O)CCCC#Cc1cnc(S(C)(=O)=O)nc1)CC3 ; CCc1c2c(nc3ccc(OCNC(=O)[C@H](C)NC(=O)[C@H](C)NC(=O)[C@H](C)NC(=O)CCCC#Cc4cnc(S(C)(=O)=O)nc4)cc13)-c1cc3c(c(=O)n1C2)COC(=O)[C@]3(O)CC]

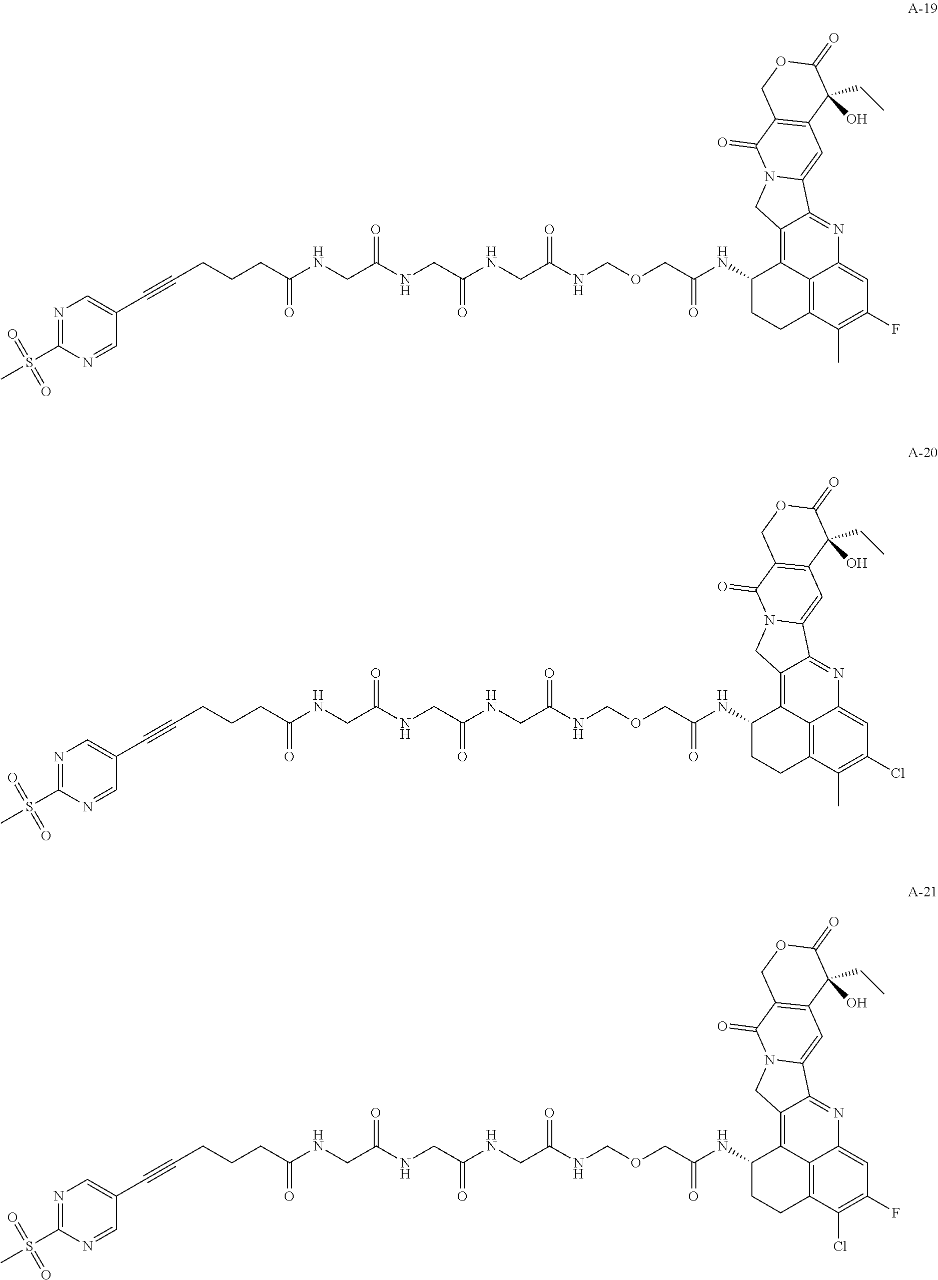 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)CNC(=O)CNC(=O)CCCC#Cc1cnc(S(C)(=O)=O)nc1)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)CNC(=O)CNC(=O)CCCC#Cc1cnc(S(C)(=O)=O)nc1)CC3 ; CC[C@@]1(O)C(=O)OCc2c1cc1n(c2=O)Cc2c-1nc1cc(F)c(Cl)c3c1c2[C@@H](NC(=O)COCNC(=O)CNC(=O)CNC(=O)CNC(=O)CCCC#Cc1cnc(S(C)(=O)=O)nc1)CC3]

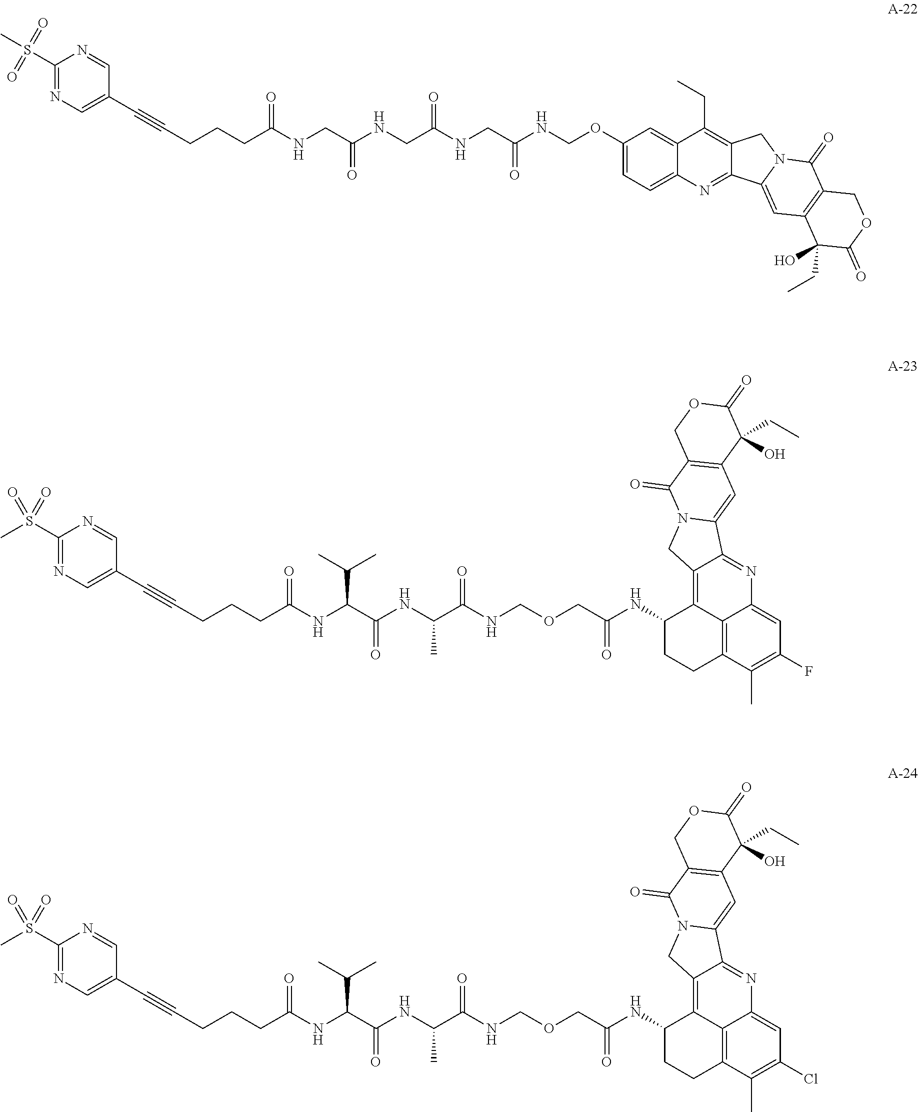 [SMILES: CCc1c2c(nc3ccc(OCNC(=O)CNC(=O)CNC(=O)CNC(=O)CCCC#Cc4cnc(S(C)(=O)=O)nc4)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@@H](NC(=O)CCCC#Cc1cnc(S(C)(=O)=O)nc1)C(C)C)CC3 ; CC[C@@]1(O)C(=O)OCc2c1cc1n(c2=O)Cc2c-1nc1cc(Cl)c(C)c3c1c2[C@@H](NC(=O)COCNC(=O)[C@H](C)NC(=O)[C@@H](NC(=O)CCCC#Cc1cnc(S(C)(=O)=O)nc1)C(C)C)CC3]

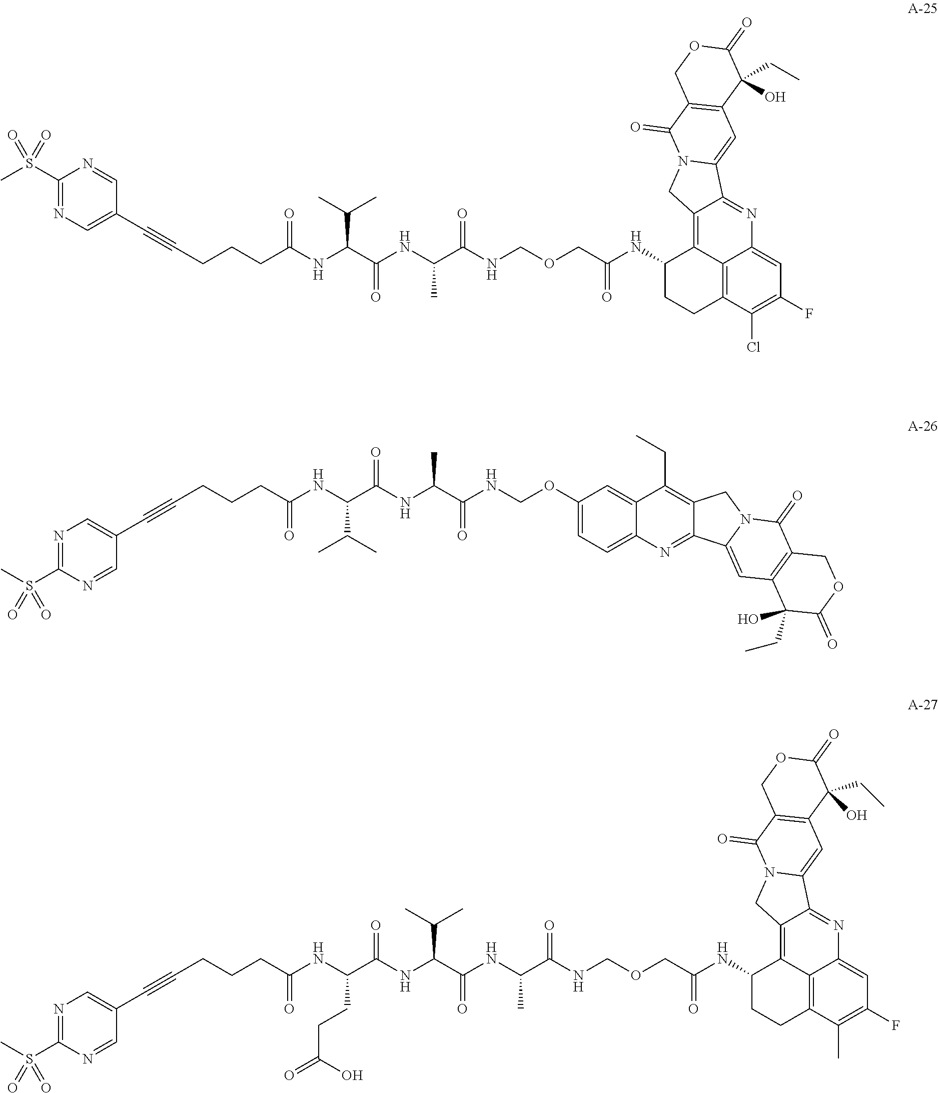 [SMILES: CC[C@@]1(O)C(=O)OCc2c1cc1n(c2=O)Cc2c-1nc1cc(F)c(Cl)c3c1c2[C@@H](NC(=O)COCNC(=O)[C@H](C)NC(=O)[C@@H](NC(=O)CCCC#Cc1cnc(S(C)(=O)=O)nc1)C(C)C)CC3 ; CCc1c2c(nc3ccc(OCNC(=O)[C@H](C)NC(=O)[C@@H](NC(=O)CCCC#Cc4cnc(S(C)(=O)=O)nc4)C(C)C)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@@H](NC(=O)[C@H](CCC(=O)O)NC(=O)CCCC#Cc1cnc(S(C)(=O)=O)nc1)C(C)C)CC3]

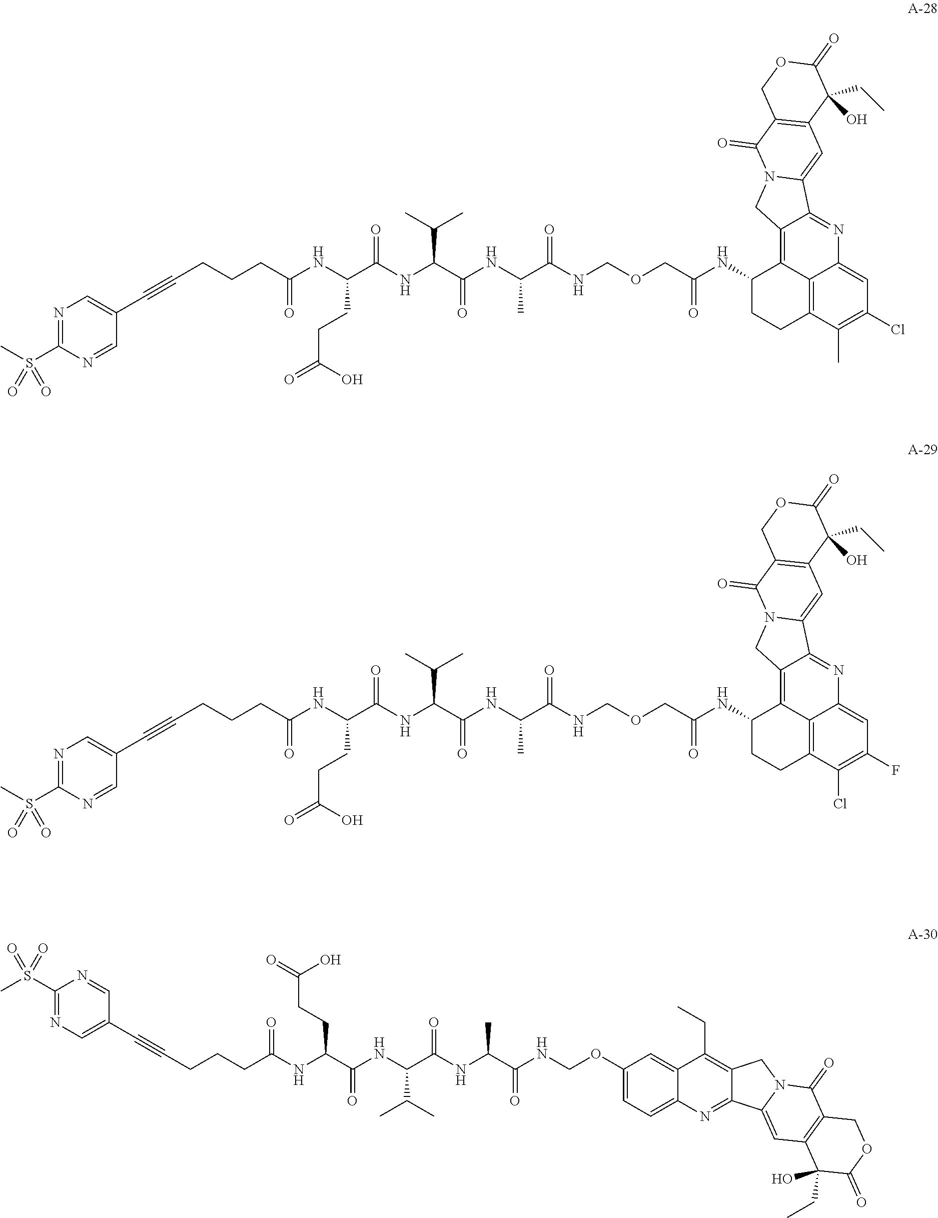 [SMILES: CC[C@@]1(O)C(=O)OCc2c1cc1n(c2=O)Cc2c-1nc1cc(Cl)c(C)c3c1c2[C@@H](NC(=O)COCNC(=O)[C@H](C)NC(=O)[C@@H](NC(=O)[C@H](CCC(=O)O)NC(=O)CCCC#Cc1cnc(S(C)(=O)=O)nc1)C(C)C)CC3 ; CC[C@@]1(O)C(=O)OCc2c1cc1n(c2=O)Cc2c-1nc1cc(F)c(Cl)c3c1c2[C@@H](NC(=O)COCNC(=O)[C@H](C)NC(=O)[C@@H](NC(=O)[C@H](CCC(=O)O)NC(=O)CCCC#Cc1cnc(S(C)(=O)=O)nc1)C(C)C)CC3 ; CCc1c2c(nc3ccc(OCNC(=O)[C@H](C)NC(=O)[C@@H](NC(=O)[C@H](CCC(=O)O)NC(=O)CCCC#Cc4cnc(S(C)(=O)=O)nc4)C(C)C)cc13)-c1cc3c(c(=O)n1C2)COC(=O)[C@]3(O)CC]

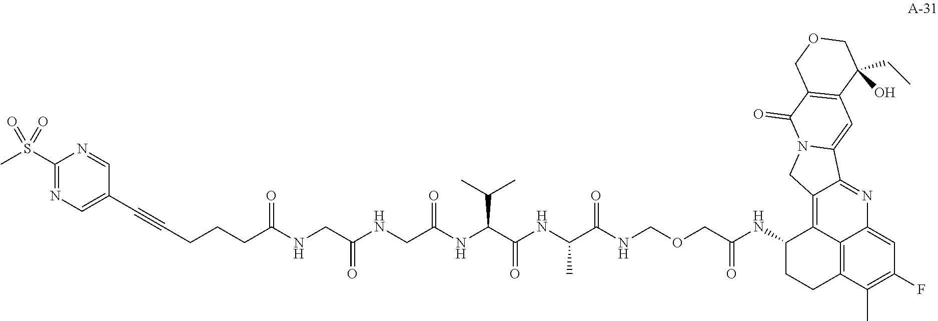 [SMILES: CC[C@@]1(O)COCc2c1cc1n(c2=O)Cc2c-1nc1cc(F)c(C)c3c1c2[C@@H](NC(=O)COCNC(=O)[C@H](C)NC(=O)[C@@H](NC(=O)CNC(=O)CNC(=O)CCCC#Cc1cnc(S(C)(=O)=O)nc1)C(C)C)CC3]

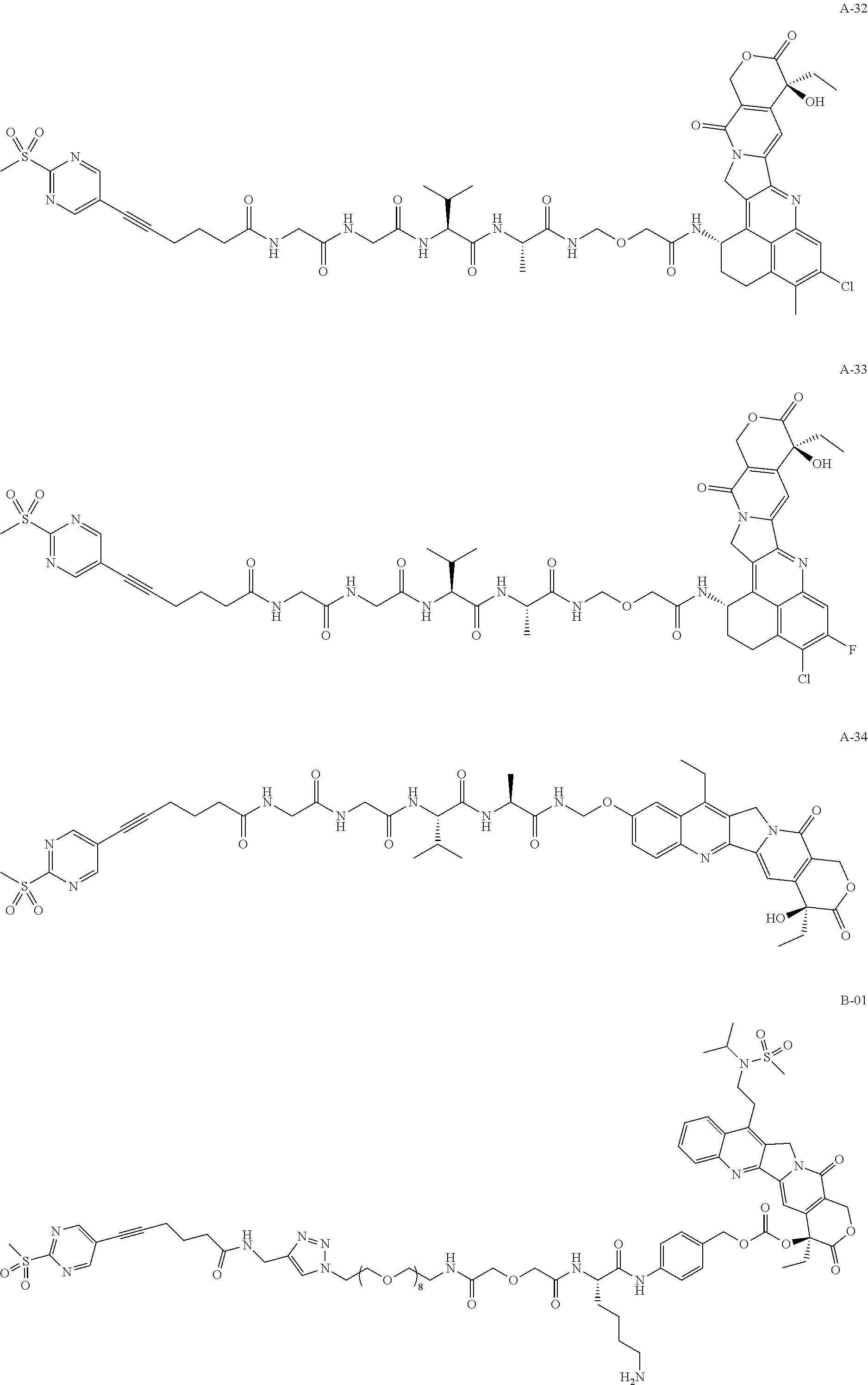

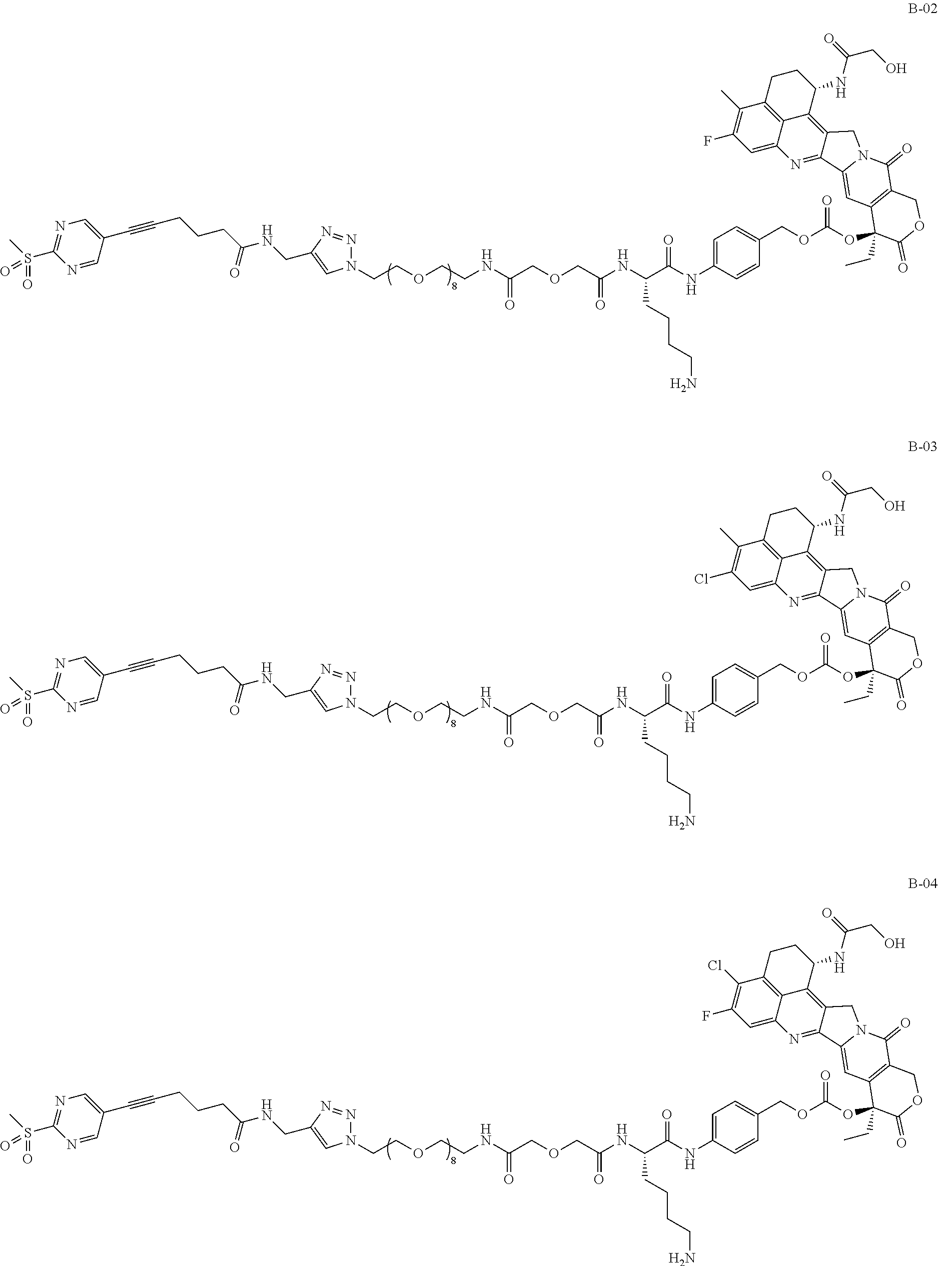

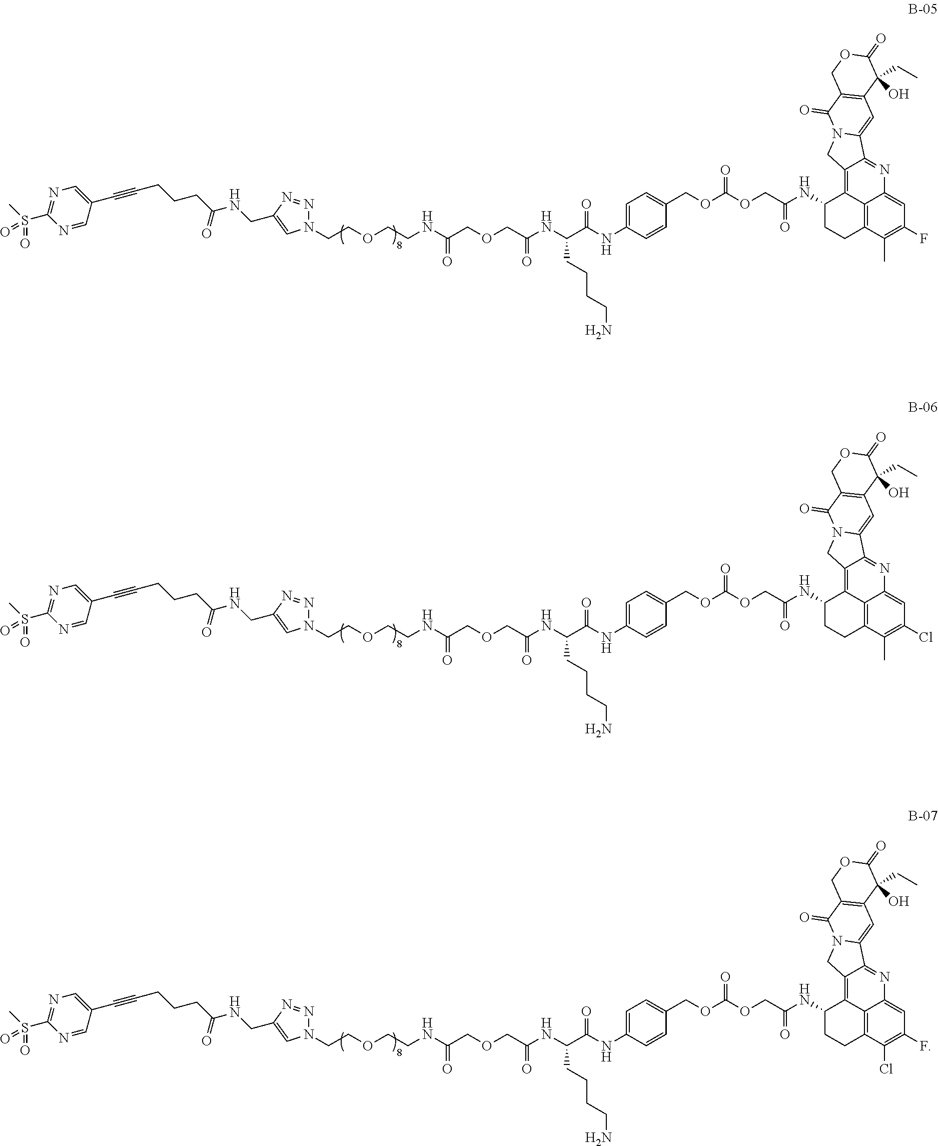

In the drug-linker of the present disclosure, the groups of all embodiments can be appropriately selected for any combination to obtain different general formula ranges or specific schemes. These ranges and schemes belong to the present disclosure. the present disclosure encompasses the drug-linkers obtained by any combination of various embodiments.

### Pharmaceutical Composition

In another aspect, the present disclosure provides a pharmaceutical composition comprising the antibody-drug conjugate as described herein or a composition as described herein, and one or more pharmaceutical excipients.

In some embodiments, the ADCs provided herein are formulated in a unit injectable form together with a pharmaceutically acceptable parenteral vehicle for parenteral use, for example, bolus injection, intravenous injection, intratumoral injection, etc. Optionally, an antibody-drug conjugate having a desired purity is mixed, in the form of a lyophilized agent or a solution, with a pharmaceutically acceptable diluent, carrier, excipient or stabilizer (Remington's Pharmaceutical Sciences (1980) 16^{th} edition, Osol, A. Ed.). The antibody-drug conjugate described herein or the pharmaceutical composition comprising the antibody-drug conjugate can be administered by any route appropriate for the subject to be treated.

### Therapeutic Uses of the ADCs

In some embodiments, the antibody-drug conjugate or the pharmaceutical composition described herein meets one or more of the following:
(1) having an excellent binding activity to CDH3;
(2) having an ability to efficiently deliver a drug to CDH3;
(3) having a better plasma stability;
(4) inhibiting cell (such as, tumor cell) proliferation;
(5) inhibiting tumor growth;
(6) inducing and/or increasing antibody-dependent cell cytotoxicity; and
(7) inhibiting CHD3-mediated signaling.

In another aspect, provided herein is the use of the antibody-drug conjugates or the pharmaceutical compositions described herein to prevent or treat a variety of diseases or conditions, for example, CDH3-mediated diseases or conditions. In some embodiments, the CDH3-mediated disease or condition is tumor, for example, CDH3-positive tumor. In some embodiments, the tumor is selected from colorectal cancer, gastric cancer, lung cancer (e.g., non-small cell lung cancer), cervical cancer, pancreatic cancer, esophageal cancer, ovarian cancer, thyroid cancer, bladder cancer, endometrial cancer, breast cancer, liver cancer, prostate cancer, skin cancer, pharyngeal cancer, and bile duct cancer, or any combination thereof. In some embodiments, the tumor is selected from lung cancer (e.g., non-small cell lung cancer), breast cancer, pancreatic cancer, and skin cancer.

The present disclosure also provides use of the antibody-drug conjugate, the composition or the pharmaceutical composition disclosed herein in the preparation of a medicament for preventing or treating a CDH3-mediated disease or condition.

Also provided is a method for preventing or treating a CDH3-mediated disease or condition, comprising administering to a subject in need thereof an effective amount of the antibody-drug conjugate, the composition or the pharmaceutical composition disclosed herein.

The ADC of the disclosure and the pharmaceutical composition of the disclosure can be formulated into any dosage form known in the medical field, for example, tablet, pill, suspension, emulsion, solution, gel, capsule, powder, granule, elixir, lozenge, suppository, injection (including solution for injection, sterile powder for injection and concentrated solution for injection), inhalant, spray, etc. The preferred dosage form depends on the intended mode of administration and therapeutic use. Pharmaceutical compositions of the disclosure should be sterile and stable under the conditions of manufacture and storage. A preferred dosage form is injection. Such injection can comprise a sterile solution for injection. For example, the sterile solution for injection can be prepared by incorporating in an appropriate solvent a necessary dose of the ADC or the pharmaceutical composition of the disclosure, and optionally, simultaneously incorporating other desired ingredients (including but not limited to, pH adjusting agent, surfactant, adjuvant, ionic strength enhancer, isotonic agent, preservative, diluent, or any combination thereof), followed by filter sterilization. In addition, a sterile solution for injection can be prepared as sterile lyophilized powder (e.g., by vacuum drying or freeze-drying) for ease of storage and use. Such sterile lyophilized powder can be dispersed in a suitable carrier before use, such as sterile pyrogen-free water.

Furthermore, the ADCs of the disclosure may be presented in pharmaceutical compositions in unit dosage form for ease of administration.

The ADC and the pharmaceutical composition of the disclosure can be administered by any suitable method known in the art, including but not limited to, oral, buccal, sublingual, ocular, topical, parenteral, rectal, intrathecal, intracytoplasmic, inguinal, intravesical, topical (e.g., powder, ointment, or drops), or nasal route. However, for many therapeutic uses, the preferred route/mode of administration is parenteral (e.g., intravenous injection, subcutaneous injection, intraperitoneal injection, intramuscular injection). The skilled artisan will understand that the route and/or mode of administration will vary depending on the intended purpose. In some preferred embodiments, the ADC and the pharmaceutical composition of the disclosure are administered by intravenous injection or injection.

The pharmaceutical composition of the disclosure may comprise a “therapeutically effective amount” or “prophylactically effective amount” of the ADC of the disclosure. The “prophylactically effective amount” means an amount sufficient to prevent, arrest, or delay the occurrence of a disease. The “therapeutically effective amount” refers to an amount sufficient to cure, or at least partially prevent the disease and its complications in a patient already suffering from the disease. The therapeutically effective amount of the ADC of the disclosure may vary according to the following factors: The severity of the disease to be treated, the general state of the patient's immune system, the general condition of the patient such as age, weight and sex, the mode of administration of the drug, and other treatments administered at the same time, etc.

In the disclosure, dosing regimens can be adjusted to obtain the optimum desired response (e.g., a therapeutic or prophylactic response). For example, a single dose can be administered, multiple doses can be administered over time or the dose can be proportionally reduced or increased as indicated by the exigencies of the therapeutic situation.

In the disclosure, the subject may be a mammal, such as a human.

### Kits

Also provided are kits comprising one or more ADCs described herein, or pharmaceutical compositions thereof. In a specific embodiment, provided herein is a pharmaceutical pack or kit comprising one or more containers filled with one or more of the ingredients of the pharmaceutical compositions described herein, such as one or more ADCs provided herein. In certain embodiments, the kits comprise a pharmaceutical composition described herein and any prophylactic or therapeutic agent, such as those described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided are kits that can be used in the methods described herein. In certain embodiments, a kit comprises an ADC described herein, in particular embodiments, a purified ADC, in one or more containers. In a specific embodiment, kits described herein contain a substantially isolated CDH3 antigen as a control. In another specific embodiment, the kits described herein further comprise a control antibody which does not react with a CDH3 antigen. In another specific embodiment, kits described herein contain one or more elements for detecting the binding of an antibody to a CDH3 antigen (e.g., the antibody can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized CDH3 antigen. The antigen provided in the kit can also be attached to a solid support. In a more specific embodiment, the detecting means of the above-described kit includes a solid support to which a CDH3 antigen is attached. Such a kit can also include a non-attached reporter-labeled anti-human antibody or anti-mouse/rat antibody. In this embodiment, binding of the antibody to the antigen can be detected by binding of the said reporter-labeled antibody.

Abbreviations

| | |
|---|---|
| CDR | Complementarity determining region in immunoglobulin |
| | variable region |
| FR | Antibody framework region: amino acid residue other than |
| | CDR residue in antibody variable region |
| VH | Antibody heavy chain variable region |
| VL | Antibody light chain variable region |
| IgG | Immunoglobulin G |
| IMGT | Numbering system based on the international |
| | ImMunoGeneTics information system ® (IMGT) |
| | initiated by Lefranc et al., see Lefranc et al., |
| | Dev. Comparat. Immunol. 27: 55-77, 2003. |
| Kabat | Immunoglobulin aligning and numbering system proposed |
| | by Elvin A. Kabat (see, for example, Kabat et al., |
| | Sequences of Proteins of Immunological Interest, 5th |
| | Ed. Public Health Service, National Institutes of |
| | Health, Bethesda, Md., 1991). |
| Chothia | Immunoglobulin numbering system proposed by Chothia et |
| | al., which is a classical rule for identifying a CDR |
| | region boundary based on a position of a structural loop |
| | region (see, for example, Chothia & Lesk (1987) J. Mol. |
| | Biol. 196: 901-917; Chothia et al. (1989) Nature 342: |
| | 878-883). |
| AbM | AbM CDR definition mode derived from relevant research |
| | of Martin (Martin A C R, Cheetham J C, Rees A R (1989) |
| | Modelling antibody hypervariable loops: A combined |
| | algorithm. Proc Natl Acad Sci USA 86: 9268-9272). |
| Contact | Contact CDR definition mode derived from relevant research |
| | of Robert M. MacCallum et al. (R M MacCallum, A C |
| | Martin, J M Thornton (1996). Antibody-antigen |
| | interactions: contact analysis and binding site |
| | topography. Journal of Molecular Biology, 262(5), |
| | 732-745.). |
| mAb | Monoclonal antibody |
| EC₅₀ | Concentration of producing 50% efficacy or binding |
| IC₅₀ | Concentration of producing 50% inhibition |
| ELISA | Enzyme-linked immunosorbent assay |
| PCR | Polymerase chain reaction |
| HRP | Horse radish peroxidase |
| K_{D} | Equilibrium dissociation constant |
| Ka | Association rate constant |
| Kd | dissociation rate constant |
| ADCC | Antibody-dependent cell cytotoxicity |
| FACS | Flow cytometer technology |
| CDR-H1 | Complementarity determining region 1 in immunoglobulin |
| | heavy chain variable region |
| CDR-H2 | Complementarity determining region 2 in immunoglobulin |
| | heavy chain variable region |
| CDR-H3 | Complementarity determining region 3 in immunoglobulin |
| | heavy chain variable region |
| CDR-L1 | Complementarity determining region 1 in immunoglobulin |
| | light chain variable region |
| CDR-L2 | Complementarity determining region 2 in immunoglobulin |
| | light chain variable region |
| CDR-L3 | Complementarity determining region 3 in immunoglobulin |
| | light chain variable region |

### Definitions

Unless otherwise defined below, all technical terms and scientific terms used herein are intended to have the same meaning as commonly understood by those skilled in the art. The reference to the technology used herein is intended to refer to the technology generally understood in the art, including those changes in the technology or replacements of the equivalent technology, which are obvious to those skilled in the art. Furthermore, the laboratory operation steps of genomics, nucleic acid chemistry, molecular biology and the like used herein are all conventional steps widely used in the corresponding fields. Although the following terms are believed to be well understood by those skilled in the art, the following definitions are set forth to better explain the present disclosure.

The term “antibody” refers to an immunoglobulin molecule generally consisting of two pairs of polypeptide chains (each pair having one light chain (LC) and one heavy chain (HC)). An antibody light chain can be classified as kappa (κ) and lambda (λ) light chains. A heavy chain can be classified as mu (μ), delta (δ), gamma (γ), alpha (α), or epsilon (ε), and the isotypes of an antibody are defined as IgM, IgD, IgG, IgA, and IgE, respectively. Within the light chain and the heavy chain, a variable region and a constant region are attached by a “J” region of about 12 or more amino acids, and the heavy chain further comprises a “D” region of about 3 or more amino acids. Each heavy chain consists of a heavy chain variable region (VH) and a heavy chain constant region (CH). The heavy chain constant region consists of 3 domains (CH1, CH2, and CH3). Each light chain consists of a light chain variable region (VL) and a light chain constant region (CL). The light chain constant region consists of one domain CL. The constant domains are not directly involved in the binding of the antibody to an antigen, but exhibit a plurality of effector functions, such as mediating the binding of immunoglobulin to a host tissue or factor, comprising various cells of the immune system (e.g., effector cells) and the first component of the classical complement system (C1q). The VH and VL regions can be further subdivided into regions having hypervariability (referred to as complementarity determining regions (CDRs)), interspersed with regions that are more conservative and referred to as framework regions (FRs). Each VH and VL is composed of 3 CDRs and 4 FRs arranged from the amino terminus to the carboxyl terminus in the following order: FR1, CDR1, FR2, CDR2, FR3, CDR3, and FR4. The variable regions (VH and VL) of each heavy chain/light chain pair respectively form antigen-binding sites. The assignment of amino acids to each region or domain can follow various numbering systems known in the art. The term “antibody” further comprises the embodiments in which the heavy chain constant region comprises a C-terminal lysine or lacks a C-terminal lysine or a C-terminal glycine-lysine dipeptide. The term further includes the embodiments in which an N-terminal amino acid of the antibody variable region has been cyclized to pyroglutamate. Thus, in a composition comprising the antibody disclosed herein, each antibody therein may independently comprise C-terminal lysine, lack C-terminal lysine, lack C-terminal glycine-lysine and/or comprise N-terminal glutamine or glutamic acid, or cyclization of the N-terminal amino acid to a pyroglutamic acid.

The term “complementarity determining region” or “CDR” refers to the amino acid residues responsible for antigen binding in the antibody variable region. The variable regions of the heavy chain and the light chain each comprise three CDRs, named CDR1, CDR2, and CDR3. The precise boundaries of these CDRs can be defined according to various numbering systems known in the art, and for example, can follow the definitions in the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al. (1989) Nature 342:878-883), the IMGT numbering system (Lefranc et al., Dev. Comparat. Immunol. 27:55-77, 2003), the AbM numbering system (Martin A CR, Cheetham J C, Rees A R (1989) Modelling antibody hypervariable loops: A combined algorithm. Proc Natl Acad Sci USA 86:9268-9272) or the Contact numbering system (R M MacCallum, A C Martin, J M Thornton (1996). Antibody-antigen interactions: contact analysis and binding site topography. Journal of Molecular Biology, 262(5), 732-745). For a given antibody, those skilled in the art will readily identify the CDRs defined by each numbering system. Furthermore, the correspondence between different numbering systems is well known to those skilled in the art (see, for example, Lefranc et al., Dev. Comparat. Immunol. 27:55-77, 2003).

In the present disclosure, the CDRs comprised in an antibody or an antigen-binding fragment thereof can be determined according to various numbering systems known in the art, for example, by the Kabat, Chothia, IMGT, Contact or AbM numbering systems.

The following general rules (disclosed at bioinf.org.uk: Professor Andrew C. R. Martin's research group) can be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

| Loops | Kabat | AbM | Chothia ¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat numbering | | | | |
| | system) 3 | | | | |
| H1 | H31--H35 | H26--H35 | H26--H32 | H30--H35 | H26--H33 |
| | (Chothia numbering | | | | |
| | system) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹ Therein, some numbering systems (especially the Chothia numbering system) vary among the publications examined.
²Except for the Contact numbering system using the Chothia or Martin (Extended Chothia) definitions, any other numbering system can be used for the CDR definitions described.
3 When the Kabat numbering system is used for numbering, the terminal of the Chothia CDR-H1 loop varies between H32 and H34, specifically depending on the loop length. (This is because the Kabat numbering system places the insertion point at H35A and H35B).
If both H35A and H35B are absent, the terminal of the loop is at H32;
if only H35A is present, the terminal of the loop is at H33; and
if both H35A and H35B are present, the terminal of the loop is at H34.

The entire amino acid sequence of VH is generally numbered according to Kabat, while the three CDRs within the variable region can be defined according to any one of the above numbering systems. In some embodiments, the amino acid sites in VH may be numbered sequentially from amino acid site 1 until the terminal of the sequence, and may also be numbered according to Kabat. Unless otherwise specified, the amino acid sites in VH and VL described herein are defined according to sequential numbering.

The numbering of amino acid sites in the heavy chain constant region can be numbered sequentially from amino acid site 1 until the terminal of the sequence, or can be numbered according to Eu. The amino acid sequence of the IgG1 heavy chain constant region has 330 amino acids, numbered sequentially from 1 to 330. The corresponding sequence numbered according to Eu starts from site 118 and terminates at site 447. Unless otherwise specified, the amino acid sites of the heavy chain and the light chain described herein are defined according to sequential numbering.

The term “framework region” or “FR” residues refer to those amino acid residues other than the CDR residues as defined above in the antibody variable region.

The term “antibody” is not limited to any particular method of producing an antibody. For example, the antibody comprises a recombinant antibody, a monoclonal antibody and a polyclonal antibody. The antibody can be an antibody of a different isotype, for example, an IgG (for example, an IgG1, IgG2, IgG3, or IgG4 subtype), IgA1, IgA2, IgD, IgE, or IgM antibody.

The term “antigen-binding fragment” of an antibody refers to a polypeptide of a fragment of an antibody, for example, a polypeptide of a fragment of a full-length antibody, which retains the ability to specifically bind to the same antigen to which the full-length antibody binds and/or competes with the full-length antibody for specifically binding to the antigen, and it is also referred to as an “antigen-binding moiety”. See generally, Fundamental Immunology, Ch. 7 (Paul, W., ed., version 2, Raven Press, N.Y. (1989), which is incorporated by reference herein in its entirety for all purposes. The antigen-binding fragment of the antibody can be produced by a recombinant DNA technology or by enzymatic or chemical cleavage of an intact antibody. Non-limiting examples of the antigen-binding fragments include Fab fragments, Fab′ fragments, F(ab)′₂ fragments, F(ab)′₃ fragments, Fd, Fv, scFv, di-scFv, (scFv)₂, disulfide-stabilized Fv proteins (“dsFv”), single domain antibodies (sdAb, nanobodies), and polypeptides that comprises at least a part of an antibody sufficient to confer specific antigen-binding ability to the polypeptide. Engineering-modified antibody variants are reviewed in Holliger et al., 2005; Nat Biotechnol, 23: 1126-1136.

The term “Fd” means an antibody fragment consisting of VH and CH1 domains; the term “dAb fragment” means an antibody fragment consisting of a VH domain (Ward et al., Nature 341:544 546 (1989)); the term “Fab fragment” means an antibody fragment consisting of VL, VH, CL, and CH1 domains; and the term “F(ab′)₂ fragment” means an antibody fragment comprising two Fab fragments attached by a disulfide bridge on a hinge region. The term “Fab′ fragment” means the fragment obtained after reducing the disulfide bonds connecting two heavy chain fragments in the F(ab′)₂ fragment, which consists of an intact light chain and the Fd fragment (consisting of the VH and CH1 domains) of the heavy chain.

The term “Fv” means an antibody fragment consisting of the VL and VH domains of a single arm of an antibody. An Fv fragment is generally considered to be the minimum antibody fragment which can form an intact antigen-binding site. It is generally believed that six CDRs confer antigen-binding specificity to an antibody. However, even a single variable region (for example, an Fd fragment, which comprises only three CDRs specific for an antigen) can also recognize and bind to an antigen, although probably at a lower affinity than an intact binding site.

The term “Fc” means an antibody fragment formed by binding second and third constant regions of a first heavy chain of an antibody to second and third constant regions of a second heavy chain via disulfide bonds. The Fc fragment of an antibody has a plurality of different functions but is not involved in antigen binding.

The term “scFv” refers to a single polypeptide chain comprising VL and VH domains, wherein the VL and VH are attached by a linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, vol. 113, Roseburg and Moore eds., Springer-Verlag, New York, pp. 269-315 (1994)). Such scFv molecules may have the general structure: NH₂-VL-Linker-VH-COOH or NH₂—VH-Linker-VL-COOH. Suitable linkers in the prior art consist of repeated GGGGS (SEQ ID NO: 150) amino acid sequences or variants thereof. For example, a linker having an amino acid sequence (GGGGS)₄ (SEQ ID NO: 151) may be used, but variants thereof may also be used (Holliger et al. (1993), Proc. Natl. Acad. Sci. USA 90: 6444-6448). Other linkers that may be used in the present disclosure are described by Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol. In some cases, there may further be a disulfide bond between the VH and VL of the scFv. In some embodiments, the VH and VL domains may be positioned relative to each other in any suitable arrangement, for example, the scFv comprising NH₂-VH-VH-COOH and NH₂-VL-VL-COOH.

The term “single-domain antibody (sdAb)” has the meaning generally understood by those skilled in the art, and refers to an antibody fragment consisting of a single monomer variable antibody domain (for example, a single heavy chain variable region) that retains the ability to specifically bind to the same antigen to which a full-length antibody binds (Holt, L. et al., Trends in Biotechnology, 21(11):484-490, 2003). The single-domain antibody is also referred to as a nanobody.

Each of the above antibody fragments retains the ability to specifically bind to the same antigen to which a full-length antibody binds, and/or competes with the full-length antibody for specifically binding to the antigen.

Herein, unless otherwise indicated clearly in the context, when referring to the term “antibody”, it comprises not only an intact antibody, but also an antigen-binding fragment of the antibody.

As used herein, the term “specifically binding” refers to a non-random binding reaction between two molecules, such as a reaction between an antibody and an antigen to which it is directed. The strength or affinity of a specific binding interaction can be expressed as an equilibrium dissociation constant (KD) or a half-maximal effect concentration (EC50) of the interaction.

An antigen-binding fragment of an antibody (for example, the above-mentioned antibody fragment) can be obtained from a given antibody (for example, the antibody provided by the present disclosure) using a conventional technology (for example, a recombinant DNA technology or an enzymatic or chemical cleavage method) known to those skilled in the art, and the antigen-binding fragment of the antibody can be screened for specificity in the same manner as for an intact antibody.

The term “murine antibody” refers to an antibody obtained by the following method: fusing B cells of an immunized mouse with myeloma cells, screening for mouse hybrid fusion cells that can both proliferate indefinitely and secrete antibodies, and then performing screening, antibody preparation and antibody purification; or it refers to an antibody secreted and produced by plasma cells formed by differentiation and proliferation of B cells after antigen invasion into the mouse.

The term “humanized antibody” refers to a genetic engineering modified non-human antibody with the amino acid sequence modified to improve the homology with the sequence of a human antibody. Generally speaking, all or part of a CDR region of a humanized antibody is from a non-human antibody (a donor antibody), and all or part of a non-CDR region (for example, a variable region FR and/or a constant region) is from human immunoglobulin (a recipient antibody). The humanized antibody generally retains the expected properties of the donor antibody, including but not limited to the antigen specificity, affinity, reactivity, ability to improve an immune cell activity, ability to enhance an immune response, etc. The donor antibody may be a mouse, rat, rabbit or non-human primate (for example, Macaca fascicularis) antibody with the expected properties (for example, the antigen specificity, affinity, reactivity, ability to improve an immune cell activity and/or ability to enhance an immune response).

As used herein, the term “variant”, in the context of a polypeptide (comprising a polypeptide), also refers to a polypeptide or a peptide comprising an amino acid sequence that has been altered by the introduction of an amino acid residue substitution, deletion, or addition. In certain cases, the term “variant” further refers to a polypeptide or a peptide that has been modified (i.e., by covalently linking any type of molecules to the polypeptide or the peptide). For example, but without limitation, the polypeptide can be modified, for example, by glycosylation, acetylation, pegylation, phosphorylation, amidation, derivatization by known protecting/blocking groups, proteolytic cleavage, attachment to cellular ligands or other proteins, etc. The technology known to those skilled in the art, including but not limited to specific chemical cleavage, acetylation, formylation, metabolic synthesis of tunicamycin, etc. can be used to produce the derived polypeptides or peptides by chemical modification. In addition, the variant has similar, same or improved functions of the polypeptide or peptide from which it is derived. In some embodiments, the variant of the present disclosure has at least 80%, at least 85%, at least 90%, at least 91%, at least 92%, at least 93%, at least 94%, at least 95%, at least 96%, at least 97%, at least 98%, at least 99% or 100% sequence identity compared to the sequence from which it is derived, or has a substitution, deletion, or addition of one, two, or more amino acids (for example, a substitution, deletion, or addition of 1, 2, 3, 4, or 5 amino acids) compared to the sequence from which it is derived; and in particular embodiments, the substitution is a conservative substitution.

The term “identity” is used to refer to the matching of sequences between two polypeptides or between two nucleic acids. When a certain position in two sequences being compared is occupied by the same base or amino acid monomer subunit (for example, a certain position in each of two DNA molecules is occupied by adenine, or a certain position in each of two polypeptides is occupied by lysine), then the molecules are identical at that position. The “percent identity” between two sequences is a function of the number of matching positions shared by two sequences divided by the number of positions being compared ×100. For example, if 6 positions among 10 positions in two sequences match, the two sequences have 60% identity. For example, the DNA sequences CTGACT and CAGGTT share 50% identity (3 positions among 6 positions match in total). Typically, two sequences are compared when they are aligned to produce the maximum identity. Such an alignment can be implemented using, for example, the method of Needleman et al. (1970) J. Mol. Biol. 48:443-453, which can be conveniently performed by a computer program, for example, the Align program (DNAstar, Inc.). The percent identity between two amino acid sequences can further be determined using the algorithm of E. Meyers and W. Miller (Comput. Appl Biosci., 4:11-17 (1988)) which has been incorporated into the ALIGN program (version 2.0) and using a PAM120 weight residue table, a gap length penalty of 12 and a gap penalty of 4. In addition, the percent identity between two amino acid sequences can be determined using the Needleman and Wunsch (J Mol Biol. 48:444-453 (1970)) algorithm which has been incorporated into the GAP program in the GCG software package (available at www.gcg.com) and using a Blossum 62 matrix or a PAM250 matrix as well as a gap weight of 16, 14, 12, 10, 8, 6, or 4 and a length weight of 1, 2, 3, 4, 5, or 6.

The term “conservative substitution” means an amino acid substitution that does not adversely affect or alter the desired properties of the protein/polypeptide comprising the amino acid sequence. For example, the conservative substitution can be introduced by standard technologies known in the art, for example, site-directed mutagenesis and PCR-mediated mutagenesis. The conservative amino acid substitution comprises a substitution of an amino acid residue with an amino acid residue having a similar side chain, for example, a substitution with a residue that is physically or functionally similar to the corresponding amino acid residue (for example, having similar sizes, shapes, charges, chemical properties, including an ability to form a covalent bond or a hydrogen bond, etc.). Families of amino acid residues having similar side chains have been defined in the art. These families comprise amino acids with basic side chains (for example, lysine, arginine, and histidine), acidic side chains (for example, aspartic acid and glutamic acid), uncharged polar side chains (for example, glycine, asparagine, glutamine, serine, threonine, tyrosine, cysteine, and tryptophan), nonpolar side chains (for example, alanine, valine, leucine, isoleucine, proline, phenylalanine, and methionine), R branched side chains (for example, threonine, valine, and isoleucine) and aromatic side chains (for example, tyrosine, phenylalanine, tryptophan, and histidine). Thus, in particular embodiments, the corresponding amino acid residue is replaced with another amino acid residue from the same side chain family. Methods for identifying conservative amino acid substitutions are well known in the art (see, for example, Brummell et al., Biochem. 32:1180-1187 (1993); Kobayashi et al. Protein Eng. 12(10):879-884 (1999); and Burks et al. Proc. Natl Acad. Set USA 94:412-417 (1997), which are incorporated herein by reference).

The writing of twenty conventional amino acids involved herein follows conventional usage. See, for example, Immunology-A Synthesis (2nd Edition, E. S. Golub and D. R. Gren, Eds., Sinauer Associates, Sunderland, Mass. (1991)), which is incorporated herein by reference. In the present disclosure, amino acids are generally represented by single-letter and three-letter abbreviations known in the art. For example, alanine can be represented by A or Ala.

The terms “include”, “comprise”, “has”, “contain”, or “involve” and other variant forms thereof herein are inclusive or open-ended and do not exclude other unlisted elements or method steps.

The term “alkyl” represents a group obtained by removing 1 hydrogen atom from linear or branched hydrocarbyl, for example, “C₁₋₂₀alkyl”, “C₁₋₁₀ alkyl”, “C₁₋₆alkyl”, “C₁₋₄alkyl”, “C₁₋₃alkyl”, etc. Specific examples include, but are not limited to, methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc.

The term “alkylene” represents a group obtained by removing 2 hydrogen atoms from linear or branched hydrocarbyl, for example, “C₁₋₂₀alkylene”, “C₁₋₁₀alkylene”, “C₃₋₁₀alkylene”, “C₅₋₈alkylene”, “C₁₋₆alkylene”, “C₁₋₄alkylene”, “C₁₋₃alkylene”, etc. Specific examples include, but are not limited to: methylene, ethylene, 1,3-propylene, 1,4-butylene, 1,5-pentylene, or 1,6-hexylene, etc.

The term “alkenylene” refers to a divalent group obtained by losing two hydrogen atoms from linear or branched hydrocarbyl containing at least one carbon-carbon double bond, including, for example, “C₂₋₂₀alkenylene”, “C₃₋₁₀alkenylene”, “C₅₋₈-alkenylene”, etc. The examples thereof include, but are not limited to: vinylene, 1-propenylene, 2-propenylene, 1-butenylene, 2-butenylene, 1,3-butadienylene, 1-pentenylene, 2-pentenylene, 3-pentenylene, 1,3-pentadienylene, 1,4-pentadienylene, 1-hexenylene, 2-hexenylene, 3-hexenylene, 1,4-hexadienylene, etc.

The term “alkynylene” refers to a divalent group obtained after losing two hydrogen atoms from linear or branched hydrocarbyl containing at least one carbon-carbon triple bond, and includes, for example, “C₂₋₂₀alkynylene”, “C₃₋₁₀alkynylene”, “C₅₋₈-alkynylene”, etc. The examples thereof include, but are not limited to: ethynylene, 1-propynylene, 2-propynylene, 1-butynylene, 2-butynylene, 1,3-butadiynylene, 1-pentynylene, 2-pentynylene, 3-pentynylene, 1,3-pentadiynylene, 1,4-pentadiynylene, 1-hexynylene, 2-hexynylene, 3-hexynylene, 1,4-hexadiynylene, etc.

The term “aliphatic heterocyclic ring” refers to a saturated or partially saturated cyclic structure containing at least one ring member selected from N, O, and S. Specific examples include, but are not limited to, 5-6-membered heterocyclic aliphatic rings, 5-6-membered nitrogen-containing aliphatic heterocyclic rings, 5-6-membered oxygen-containing aliphatic heterocyclic rings, etc., for example, tetrahydrofuran, pyrrolidine, piperidine, tetrahydropyran, etc.

The term “heteroaromatic ring” refers to an aromatic ring structure containing at least one ring member selected from N, O, and S. Specific examples include, but are not limited to, 5-6-membered heteroaromatic rings, 5-6-membered nitrogen-containing heteroaromatic rings, 5-6-membered oxygen-containing heteroaromatic rings, for example, furan, thiophene, pyrrole, thiazole, isothiazole, thiadiazole, oxazole, isoxazole, oxadiazole, imidazole, pyrazole, 1,2,3-triazole, 1,2,4-triazole, 1,2,3-oxadiazole, 1,2,4-oxadiazole, 1,2,5-oxadiazole, 1,3,4-oxadiazole, pyridine, pyrimidine, pyridazine, pyrazine, 1,2,3-triazine, 1,3,5-triazine, 1,2,4,5-tetrazine, etc.

The term “aromatic ring system” refers to a monocyclic or polycyclic system comprising at least one aromatic ring (for example, a phenyl ring, etc.) or heteroaromatic ring (for example, a pyrimidine ring, etc.), two or more aromatic rings and/or heteroaromatic rings may form a fused ring or be attached by a single bond (for example, dipyrimidinylphenyl, etc.), and the aromatic ring system may in a bivalence or higher valence state (for example, trivalence or tetravalence), for example, a 5-20-membered aromatic ring system.

As used herein, the term “suitable substituent” refers to modifications that can be made to a compound by those skilled in the art according to the needs of the compound substituent. “Suitable substituents” include oxo (═O), halogen, cyano, NR⁸R⁹, carboxyl, sulfhydryl, hydroxy, ester groups (for example, —C₁₋₆alkyl-C(═O)—OC₁₋₆alkyl), C₁₋₆alkyl, C₂₋₆alkenyl, C₂₋₆alkynyl, C₁₋₆alkyl-O—C₁₋₆alkyl, C₁₋₆ haloalkyl, C₁₋₆alkoxy, C₃₋₆cycloalkyl, 3-10-membered heterocyclyl, 5-10-membered heteroaryl, C₆₋₁₀aryl, benzyl, benzyl substituted with hydroxy, indolylmethylene and C₁₋₆haloalkoxy, wherein R⁸ and R⁹ are each independently selected from H, C₁₋₆alkyl, C₃₋₆ cycloalkyl, 3-10-membered heterocyclyl, 5-10-membered heteroaryl, C₆₋₁₀aryl, C₁₋₆alkoxy, C₁₋₆haloalkyl, C₁₋₆haloalkoxy, halogen, hydroxy, carboxyl and ester groups (for example, —C₁₋₆alkyl-C(═O)—OC₁₋₆alkyl).

The term “substituted” refers to the replacement of one or more (for example, 1, 2, 3, 4, or 5) hydrogen atoms on a specified compound or structural fragment with a substituent, provided that the normal valence of the specified atom in the current situation is not exceeded and the substitution forms a stable compound. A combination of substituents and/or variables is permitted only if such combination forms a stable compound. For example, the substituents each independently consist of one or more of the following structures: NR⁸R⁹, —O—, —S—, —NR′—, halogen, —CN, —OH, —SH, —NH₂, —NO₂, —C(O)—, —CN, =O, C₁-C₆alkyl (alkylene), C₁-C₆ haloalkyl (haloalkylene), C₁-C₆alkoxy, C₂-C₆alkenyl (alkenylene), C₂-C₆alkynyl (alkynylene), C₃-C₈cycloalkyl (cycloalkylene), 3-10-member heterocyclyl (heterocyclylene), C₆-C₁₀aryl (arylene), 5-10-member heteroaryl (heteroarylene), etc., wherein R⁸, R⁹, and R′ are as defined above. For example, the substituents may be suitable substituents as described above.

Whether or not explicitly stated, the numerical values of the present disclosure are modified by the term “about”. The term “about” refers to a range within ±20%, ±10%, ±5%, or ±2% of the stated value.

The information on the sequences involved in the present disclosure is described in the following table:

Description of the Sequences

| SEQ ID NO: | Description | Sequence |
|---|---|---|
| 1 | VH of Ab-B | QVQLQQPGTELVKPGASVKLSCQASGFTFTSYWM |
| | | HWVKQRPGQGLAWIGNINLSYGNTNYNEKFKNKA |
| | | TLTVDKSSSTAYMQLSGLTSEDSAVYYCARGFSYF |
| | | DYWGQGTTLTVSS |
| 2 | VL of Ab-B | DIVMTQSHKFMSTSLGDRVNITCKASQNVDTDVS |
| | | WYQQKPGKSPKPLIYWASNRFTGVPDRFTGSGSGT |
| | | DFTLTINNVQSEDLADYFCEQYSSYPFTFGTGTKLEI |
| | | K |
| 3 | VH of Ab-BHZ\Ab- | QVQLVQSGAEVKKPGASVKVSCKASGFTFTSYWM |
| | BHZm | HWVRQAPGQGLEWIGNIQLSYGNTNYAQKFQGRV |
| | | TLTVDKSSSTAYMELSSLRSEDTAVYYCARGFSYF |
| | | DYWGQGTLVTVSS |
| 4 | VL of Ab-BHZ\Ab- | DIQMTQSPSSLSASVGDRVTITCKASQNVDTDVSW |
| | BHZm | YQQKPGKAPKSLIYWASNRFTGVPSRFSGSGSGTD |
| | | FTLTISSLQPEDFATYFCEQYSSYPFTFGQGTKVEIK |
| 5 | VH of Ab-C | DVQLVESGGGLVQPGGSRKLSCAASGFTFTSFGMH |
| | | WVRQTPEKGLEWVAYISSVSNIIYYADTVKGRFTIS |
| | | RDNPKNTLFLQMTSLRSEDTAMYYCARDGKYYYY |
| | | TMDYWGQGTSVTVSS |
| 6 | VL of Ab-C | QAVVTQESALTTSPGETVTLTCRSSTGAVTTTNYA |
| | | NWVQEKPDHLFTGLIGGTNNRTPGVPARFSGSLIG |
| | | DKAALTITGAQTEDEAVYFCALWYSNHWVFGGGT |
| | | KLTVL |
| 7 | VH of Ab-CHZ | EVQLVESGGGLVQPGGSLRLSCAASGFTFTSFGMH |
| | | WVRQAPGKGLEWVAYISSVSNIIYYADSVKGRFTIS |
| | | RDNAKNSLYLQMNSLRAEDTAVYYCARDGKYYY |
| | | YTMDYWGQGTLVTVSS |
| 8 | VL of Ab-CHZ | QTVVTQEPSLTVSPGGTVTLTCRSSTGAVTTTNYA |
| | | NWVQQKPGQAFRALIGGTNQRTPWTPARFSGSLLG |
| | | GKAALTLSGVQPEDEAEYYCALWYSNHWVFGGG |
| | | TKLTVL |
| 9 | VH of Ab-D | QVQLQQSGAELMKPGASVKISCKATGYTFSSYWIE |
| | | WVKQRPGHGLERIGEILPGSGNTNYNEKFKGKATF |
| | | TADTSSNTAYMQLSSLTAEDSAVYYCARREYFGSR |
| | | DYWGQGTTLTVSS |
| 10 | VL of Ab-D | DIQMTQTTSSLSASLGDRVTISCRASQDISNYLNWY |
| | | QQKPDGTVKLLIYYTSRLHSGVPSRFSGSGSGTDYS |
| | | LTISNLEQEDIATYFCQQGNTLPYAFGGGTKLEIK |
| 11 | VH of Ab-DHZ | QVQLVQSGAEVKKPGSSVKVSCKASGYTESSYWIE |
| | | WVRQAPGQGLERMGEILPGSGNTNYAQKFQGRVT |
| | | ITADKSTNTAYMELSSLRSEDTAVYYCARREYFGS |
| | | RDYWGQGTLVTVSS |
| 12 | VL of Ab-DHZ | DIQMTQSPSSLSASVGDRVTITCQASQDISNYLNWY |
| | | QQKPEGAVKLLIYYTSRLHTGVPSRFSGSGSGTDYT |
| | | FTISSLQPEDIATYYCQQGNTLPYAFGGGTKVEIK |
| 13 | VH of Ab-A | QVQLQQSDAELVKPGVSVKISCKVSGSTFTDHTIH |
| | | WMKQRPEQGLEWIGYIYPTDGSTQYNEKFRGKAT |
| | | LTADRSSNTAYMQLNSLTYEDSAVYFSSLRPLFNVI |
| | | KHWGQGTSVTVSS |
| 14 | VL of Ab-A | DIVMTQSPSSLTVTAGEKVTMSCKSSQSLLDSGNH |
| | | KNFLTWYQQKPGQPPKLLIFWASTRETGVPDRFTG |
| | | SGSGIDFTLTITSVQAEDLAVYYCQNDYRYPLTFGA |
| | | GTKLELK |
| 15 | VH of Ab-AHZ | QVQLVQSGAEVKKPGASVKVSCKVSGSTFTDHTIH |
| | | WMRQAPGQGLEWMGYIYPTSGSTQYAQKFQGRV |
| | | TLTADRSTNTAYMELSSLRSEDTAVYFCSLRPLFNV |
| | | IKHWGQGTLVTVSS |
| 16 | VL of Ab-AHZ | DIVMTQSPDSLAVSLGERATINCKSSQSLLDRGNHK |
| | | NFLTWYQQKPGQPPKLLIFWASTRETGVPDRFSGS |
| | | GSGIDFTLTISSLQAEDVAVYYCQNDYRYPLTFGQG |
| | | TKLEIK |
| 17 | Ab-B/Ab-BHZ Chothia | GFTFTSY |
| | CDR-H1 | |
| 18 | Ab-B/Ab-BHZ AbM | GFTFTSYWMH |
| | CDR-H1 | |
| 19 | Ab-B/Ab-BHZ Kabat | SYWMH |
| | CDR-H1 | |
| 20 | Ab-B/Ab-BHZ Contact | TSYWMH |
| | CDR-H1 | |
| 21 | Ab-B/Ab-BHZ IMGT | GFTFTSYW |
| | CDR-H1 | |
| 22 | Ab-B Chothia CDR-H2 | NLSYGN |
| 23 | Ab-B AbM CDR-H2 | NINLSYGNTN |
| 24 | Ab-B Kabat CDR-H2 | NINLSYGNTNYNEKFKN |
| 25 | Ab-B Contact CDR-H2 | WIGNINLSYGNTN |
| 26 | Ab-B IMGT CDR-H2 | INLSYGNT |
| 27 | Ab-BHZ Chothia CDR-H2 | QLSYGN |
| 28 | Ab-BHZ AbM CDR-H2 | NIQLSYGNTN |
| 29 | Ab-BHZ Kabat CDR-H2 | NIQLSYGNTNYAQKFQG |
| 30 | Ab-BHZ Contact CDR-H2 | WIGNIQLSYGNTN |
| 31 | Ab-BHZ IMGT CDR-H2 | IQLSYGNT |
| 32 | Ab-B/Ab-BHZ | GFSYFDY |
| | Chothia/AbM/Kabat | |
| | CDR-H3 | |
| 33 | Ab-B/Ab-BHZ Contact | ARGFSYFD |
| | CDR-H3 | |
| 34 | Ab-B/Ab-BHZ IMGT | ARGFSYFDY |
| | CDR-H3 | |
| 35 | Ab-B/Ab-BHZ | KASQNVDTDVS |
| | Chothia/AbM/Kabat | |
| | CDR-L1 | |
| 36 | Ab-B/Ab-BHZ Contact | DTDVSWY |
| | CDR-L1 | |
| 37 | Ab-B/Ab-BHZ IMGT | QNVDTD |
| | CDR-L1 | |
| 38 | Ab-B/Ab-BHZ | WASNRFT |
| | Chothia/AbM/Kabat | |
| | CDR-L2 | |
| 39 | Ab-B Contact CDR-L2 | PLIYWASNRF |
| 40 | Ab-BHZ Contact CDR-L2 | SLIYWASNRF |
| 41 | Ab-B/Ab-BHZ/Ab- | WAS |
| | A/Ab-AHZ IMGT CDR-L2 | |
| 42 | Ab-B/Ab-BHZ | EQYSSYPFT |
| | Chothia/AbM/Kabat/IM | |
| 43 | Ab-B/Ab-BHZ Contact | EQYSSYPF |
| | CDR-L3 | |
| 44 | Ab-C/Ab-CHZ Chothia | GFTFTSF |
| | CDR-H1 | |
| 45 | Ab-C/Ab-CHZ AbM | GFTFTSFGMH |
| | CDR-H1 | |
| 46 | Ab-C/Ab-CHZ Kabat | SFGMH |
| | CDR-H1 | |
| 47 | Ab-C/Ab-CHZ Contact | TSFGMH |
| | CDR-H1 | |
| 48 | Ab-C/Ab-CHZ IMGT | GFTFTSFG |
| | CDR-H1 | |
| 49 | Ab-C/Ab-CHZ Chothia | SSVSNI |
| | CDR-H2 | |
| 50 | Ab-C/Ab-CHZ AbM | YISSVSNIIY |
| | CDR-H2 | |
| 51 | Ab-C Kabat CDR-H2 | YISSVSNIIYYADTVKG |
| 52 | Ab-CHZ Kabat CDR-H2 | YISSVSNIIYYADSVKG |
| 53 | Ab-C/Ab-CHZ Contact | WVAYISSVSNIIY |
| | CDR-H2 | |
| 54 | Ab-C/Ab-CHZ IMGT | ISSVSNII |
| | CDR-H2 | |
| 55 | Ab-C/Ab-CHZ | DGKYYYYTMDY |
| | Chothia/AbM/Kabat | |
| | CDR-H3 | |
| 56 | Ab-C/Ab-CHZ Contact | ARDGKYYYYTMD |
| | CDR-H3 | |
| 57 | Ab-C/Ab-CHZ IMGT | ARDGKYYYYTMDY |
| | CDR-H3 | |
| 58 | Ab-C/Ab-CHZ | RSSTGAVTTTNYAN |
| | Chothia/AbM/Kabat | |
| | CDR-L1 | |
| 59 | Ab-C/Ab-CHZ Contact | VTTTNYANWV |
| | CDR-L1 | |
| 60 | Ab-C/Ab-CHZ IMGT | TGAVTTTNY |
| | CDR-L1 | |
| 61 | Ab-C | GTNNRTP |
| | Chothia/AbM/Kabat | |
| | CDR-L2 | |
| 62 | Ab-C Contact CDR-L2 | GLIGGTNNRT |
| 63 | Ab-CHZ | GTNQRTP |
| | Chothia/AbM/Kabat | |
| | CDR-L2 | |
| 64 | Ab-CHZ Contact CDR-L2 | ALIGGTNQRT |
| 65 | Ab-C/Ab-CHZ IMGT | GTN |
| | CDR-L2 | |
| 66 | Ab-C/Ab-CHZ | ALWYSNHWV |
| | Chothia/AbM/Kabat/IMGT | |
| | CDR-L3 | |
| 67 | Ab-C/Ab-CHZ Contact | ALWYSNHW |
| | CDR-L3 | |
| 68 | Ab-D/Ab-DHZ Chothia | GYTFSSY |
| | CDR-H1 | |
| 69 | Ab-D/Ab-DHZ AbM | GYTFSSYWIE |
| | CDR-H1 | |
| 70 | Ab-D/Ab-DHZ Kabat | SYWIE |
| | CDR-H1 | |
| 71 | Ab-D/Ab-DHZ Contact | SSYWIE |
| | CDR-H1 | |
| 72 | Ab-D/Ab-DHZ IMGT | GYTFSSYW |
| | CDR-H1 | |
| 73 | Ab-D/Ab-DHZ Chothia | LPGSGN |
| | CDR-H2 | |
| 74 | Ab-D/Ab-DHZ AbM | EILPGSGNTN |
| 75 | Ab-D Kabat CDR-H2 | EILPGSGNTNYNEKFKG |
| 76 | Ab-D Contact CDR-H2 | RIGEILPGSGNTN |
| 77 | Ab-DHZ Kabat CDR-H2 | EILPGSGNTNYAQKFQG |
| 78 | Ab-DHZ Contact CDR-H2 | RMGEILPGSGNTN |
| 79 | Ab-D/Ab-DHZ IMGT | ILPGSGNT |
| | CDR-H2 | |
| 80 | Ab-D/Ab-DHZ | REYFGSRDY |
| | Chothia/AbM/Kabat | |
| | CDR-H3 | |
| 81 | Ab-D/Ab-DHZ Contact | ARREYFGSRD |
| | CDR-H3 | |
| 82 | Ab-D/Ab-DHZ IMGT | ARREYFGSRDY |
| | CDR-H3 | |
| 83 | Ab-D | RASQDISNYLN |
| | Chothia/AbM/Kabat | |
| | CDR-L1 | |
| 84 | Ab-DHZ | QASQDISNYLN |
| | Chothia/AbM/Kabat | |
| | CDR-L1 | |
| 85 | Ab-D/Ab-DHZ Contact | SNYLNWY |
| | CDR-L1 | |
| 86 | Ab-D/Ab-DHZ IMGT | QDISNY |
| | CDR-L1 | |
| 87 | Ab-D | YTSRLHS |
| | Chothia/AbM/Kabat | |
| | CDR-L2 | |
| 88 | Ab-DHZ | YTSRLHT |
| | Chothia/AbM/Kabat | |
| | CDR-L2 | |
| 89 | Ab-D/Ab-DHZ Contact | LLIYYTSRLH |
| | CDR-L2 | |
| 90 | Ab-D/Ab-DHZ IMGT | YTS |
| | CDR-L2 | |
| 91 | Ab-D/Ab-DHZ | QQGNTLPYA |
| | Chothia/AbM/Kabat/IM | |
| | GT CDR-L3 | |
| 92 | Ab-D/Ab-DHZ Contact | QQGNTLPY |
| | CDR-L3 | |
| 93 | Ab-A/Ab-AHZ Chothia | GSTFTDH |
| | CDR-H1 | |
| 94 | Ab-A/Ab-AHZ AbM | GSTFTDHTIH |
| | CDR-H1 | |
| 95 | Ab-A/Ab-AHZ Kabat | DHTIH |
| | CDR-H1 | |
| 96 | Ab-A/Ab-AHZ Contact | TDHTIH |
| | CDR-H1 | |
| 97 | Ab-A/Ab-AHZ IMGT | GSTFTDHT |
| | CDR-H1 | |
| 98 | Ab-A Chothia CDR-H2 | YPTDGS |
| 99 | Ab-A AbM CDR-H2 | YIYPTDGSTQ |
| 100 | Ab-A Kabat CDR-H2 | YIYPTDGSTQYNEKFRG |
| 101 | Ab-A Contact CDR-H2 | WIGYIYPTDGSTQ |
| 102 | Ab-A IMGT CDR-H2 | IYPTDGST |
| 103 | Ab-AHZ Chothia CDR-H2 | YPTSGS |
| 104 | Ab-AHZ AbM CDR-H2 | YIYPTSGSTQ |
| 105 | Ab-AHZ Kabat CDR-H2 | YIYPTSGSTQYAQKFQG |
| 106 | Ab-AHZ Contact CDR-H2 | WMGYIYPTSGSTQ |
| 107 | Ab-AHZ IMGT CDR-H2 | IYPTSGST |
| 108 | Ab-A/Ab-AHZ | RPLFNVIKH |
| | Chothia/AbM/Kabat | |
| | CDR-H3 | |
| 109 | Ab-A/Ab-AHZ Contact | SLRPLENVIK |
| | CDR-H3 | |
| 110 | Ab-A/Ab-AHZ IMGT | SLRPLFNVIKH |
| | CDR-H3 | |
| 111 | Ab-A | KSSQSLLDSGNHKNFLT |
| | Chothia/AbM/Kabat | |
| | CDR-L1 | |
| 112 | Ab-A Contact CDR-L1 | LDSGNHKNFLTWY |
| 113 | Ab-A IMGT CDR-L1 | QSLLDSGNHKNF |
| 114 | Ab-AHZ | KSSQSLLDRGNHKNFLT |
| | Chothia/AbM/Kabat | |
| | CDR-L1 | |
| 115 | Ab-AHZ Contact CDR-L1 | LDRGNHKNFLTWY |
| 116 | Ab-AHZ IMGT CDR-L1 | QSLLDRGNHKNF |
| 117 | Ab-A/Ab-AHZ | WASTRET |
| | Chothia/AbM/Kabat | |
| | CDR-L2 | |
| 118 | Ab-A/Ab-AHZ Contact | LLIFWASTRE |
| | CDR-L2 | |
| 119 | Ab-A/Ab-AHZ | QNDYRYPLT |
| | Chothia/AbM/Kabat/IM | |
| | GT CDR-L3 | |
| 120 | Ab-A/Ab-AHZ Contact | QNDYRYPL |
| | CDR-L3 | |
| 121 | CH of Ab-A/Ab- | ASTKGPSVFPLAPSSKSTSGGTAALGCLVKDYFPEP |
| | AHZ/Ab-B/Ab-BHZ/Ab- | VTVSWNSGALTSGVHTFPAVLQSSGLYSLSSVVTV |
| | C/Ab-CHZ/Ab-D/Ab- | PSSSLGTQTYICNVNHKPSNTKVDKKVEPKSCDKT |
| | DHZ | HTCPPCPAPELLGGPSVFLFPPKPKDTLMISRTPEVT |
| | | CVVVDVSHEDPEVKFNWYVDGVEVHNAKTKPREE |
| | | QYNSTYRVVSVLTVLHQDWLNGKEYKCKVSNKA |
| | | LPAPIEKTISKAKGQPREPQVYTLPPSRDELTKNQVS |
| | | LTCLVKGFYPSDIAVEWESNGQPENNYKTTPPVLD |
| | | SDGSFFLYSKLTVDKSRWQQGNVFSCSVMHEALH |
| | | NHYTQKSLSLSPGK |
| 122 | CH of Ab-BHZm | ASTKGPSVFPLAPSSKSTSGGTAALGCLVKDYFPEP |
| | | VTVSWNSGALTSGVHTFPAVLQSSGLYSLSSVVTV |
| | | PSSSLGTQTYICNVNHKPSNTKVDKKVEPKSCDKT |
| | | HTCPPCPAPEAAGAPSVFLFPPKPKDTLMISRTPEVT |
| | | CVVVDVSHEDPEVKFNWYVDGVEVHNAKTKPREE |
| | | QYNSTYRVVSVLTVLHQDWLNGKEYKCKVSNKA |
| | | LPAPIEKTISKAKGQPREPQVYTLPPSRDELTKNQVS |
| | | LTCLVKGFYPSDIAVEWESNGQPENNYKTTPPVLD |
| | | SDGSFFLYSKLTVDKSRWQQGNVFSCSVMHEALH |
| | | NHYTQKSLSLSPGK |
| 123 | Kappa CL | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 124 | HC of Ab-BHZ | QVQLVQSGAEVKKPGASVKVSCKASGFTFTSYWM |
| | | HWVRQAPGQGLEWIGNIQLSYGNTNYAQKFQGRV |
| | | TLTVDKSSSTAYMELSSLRSEDTAVYYCARGFSYF |
| | | DYWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKKVEPKSCDKTHTCPPCPAPELLGGPSVFLFPPK |
| | | PKDTLMISRTPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 125 | LC of Ab-BHZ\Ab- | DIQMTQSPSSLSASVGDRVTITCKASQNVDTDVSW |
| | BHZm | YQQKPGKAPKSLIYWASNRFTGVPSRFSGSGSGTD |
| | | FTLTISSLQPEDFATYFCEQYSSYPFTFGQGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 126 | HC of Ab-BHZm | QVQLVQSGAEVKKPGASVKVSCKASGFTFTSYWM |
| | | HWVRQAPGQGLEWIGNIQLSYGNTNYAQKFQGRV |
| | | TLTVDKSSSTAYMELSSLRSEDTAVYYCARGFSYF |
| | | DYWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKKVEPKSCDKTHTCPPCPAPEAAGAPSVFLFPPK |
| | | PKDTLMISRTPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 127 | HC of Ab-B | QVQLQQPGTELVKPGASVKLSCQASGFTFTSYWM |
| | | HWVKQRPGQGLAWIGNINLSYGNTNYNEKFKNKA |
| | | TLTVDKSSSTAYMQLSGLTSEDSAVYYCARGFSYF |
| | | DYWGQGTTLTVSSASTKGPSVFPLAPSSKSTSGGTA |
| | | ALGCLVKDYFPEPVTVSWNSGALTSGVHTFPAVLQ |
| | | SSGLYSLSSVVTVPSSSLGTQTYICNVNHKPSNTKV |
| | | DKKVEPKSCDKTHTCPPCPAPELLGGPSVFLFPPKP |
| | | KDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYTL |
| | | PPSRDELTKNQVSLTCLVKGFYPSDIAVEWESNGQP |
| | | ENNYKTTPPVLDSDGSFFLYSKLTVDKSRWQQGNV |
| | | FSCSVMHEALHNHYTQKSLSLSPGK |
| 128 | LC of Ab-B | DIVMTQSHKFMSTSLGDRVNITCKASQNVDTDVS |
| | | WYQQKPGKSPKPLIYWASNRFTGVPDRFTGSGSGT |
| | | DFTLTINNVQSEDLADYFCEQYSSYPFTFGTGTKLEI |
| | | KRTVAAPSVFIFPPSDEQLKSGTASVVCLLNNFYPR |
| | | EAKVQWKVDNALQSGNSQESVTEQDSKDSTYSLS |
| | | STLTLSKADYEKHKVYACEVTHQGLSSPVTKSFNR |
| | | GEC |
| 129 | HC of Ab-CHZ | EVQLVESGGGLVQPGGSLRLSCAASGFTFTSFGMH |
| | | WVRQAPGKGLEWVAYISSVSNIIYYADSVKGRFTIS |
| | | RDNAKNSLYLQMNSLRAEDTAVYYCARDGKYYY |
| | | YTMDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPS |
| | | NTKVDKKVEPKSCDKTHTCPPCPAPELLGGPSVFLF |
| | | PPKPKDTLMISRTPEVTCVVVDVSHEDPEVKENWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSRDELTKNQVSLTCLVKGFYPSDIAVEWES |
| | | NGQPENNYKTTPPVLDSDGSFFLYSKLTVDKSRWQ |
| | | QGNVFSCSVMHEALHNHYTQKSLSLSPGK |
| 130 | LC of Ab-CHZ | QTVVTQEPSLTVSPGGTVTLTCRSSTGAVTTTNYA |
| | | NWVQQKPGQAFRALIGGTNQRTPWTPARFSGSLLG |
| | | GKAALTLSGVQPEDEAEYYCALWYSNHWVFGGG |
| | | TKLTVLGQPKAAPSVTLFPPSSEELQANKATLVCLI |
| | | SDFYPGAVTVAWKADSSPVKAGVETTTPSKQSNN |
| | | KYAASSYLSLTPEQWKSHRSYSCQVTHEGSTVEKT |
| | | VAPTECS |
| 131 | HC of Ab-C | DVQLVESGGGLVQPGGSRKLSCAASGFTFTSFGMH |
| | | WVRQTPEKGLEWVAYISSVSNIIYYADTVKGRFTIS |
| | | RDNPKNTLFLQMTSLRSEDTAMYYCARDGKYYYY |
| | | TMDYWGQGTSVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPELLGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQV |
| | | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| 132 | LC of Ab-C | QAVVTQESALTTSPGETVTLTCRSSTGAVTTTNYA |
| | | NWVQEKPDHLFTGLIGGTNNRTPGVPARFSGSLIG |
| | | DKAALTITGAQTEDEAVYFCALWYSNHWVFGGGT |
| | | KLTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKTV |
| | | APTECS |
| 133 | HC of Ab-DHZ | QVQLVQSGAEVKKPGSSVKVSCKASGYTESSYWIE |
| | | WVRQAPGQGLERMGEILPGSGNTNYAQKFQGRVT |
| | | ITADKSTNTAYMELSSLRSEDTAVYYCARREYFGS |
| | | RDYWGQGTLVTVSSASTKGPSVFPLAPSSKSTSGG |
| | | TAALGCLVKDYFPEPVTVSWNSGALTSGVHTFPAV |
| | | LQSSGLYSLSSVVTVPSSSLGTQTYICNVNHKPSNT |
| | | KVDKKVEPKSCDKTHTCPPCPAPELLGGPSVFLFPP |
| | | KPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQV |
| | | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| 134 | LC of Ab-DHZ | DIQMTQSPSSLSASVGDRVTITCQASQDISNYLNWY |
| | | QQKPEGAVKLLIYYTSRLHTGVPSRFSGSGSGTDYT |
| | | FTISSLQPEDIATYYCQQGNTLPYAFGGGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 135 | HC of Ab-D | QVQLQQSGAELMKPGASVKISCKATGYTFSSYWIE |
| | | WVKQRPGHGLERIGEILPGSGNTNYNEKFKGKATF |
| | | TADTSSNTAYMQLSSLTAEDSAVYYCARREYFGSR |
| | | DYWGQGTTLTVSSASTKGPSVFPLAPSSKSTSGGTA |
| | | ALGCLVKDYFPEPVTVSWNSGALTSGVHTFPAVLQ |
| | | SSGLYSLSSVVTVPSSSLGTQTYICNVNHKPSNTKV |
| | | DKKVEPKSCDKTHTCPPCPAPELLGGPSVFLFPPKP |
| | | KDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYTL |
| | | PPSRDELTKNQVSLTCLVKGFYPSDIAVEWESNGQP |
| | | ENNYKTTPPVLDSDGSFFLYSKLTVDKSRWQQGNV |
| | | FSCSVMHEALHNHYTQKSLSLSPGK |
| 136 | LC of Ab-D | DIQMTQTTSSLSASLGDRVTISCRASQDISNYLNWY |
| | | QQKPDGTVKLLIYYTSRLHSGVPSRFSGSGSGTDYS |
| | | LTISNLEQEDIATYFCQQGNTLPYAFGGGTKLEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 137 | HC of Ab-AHZ | QVQLVQSGAEVKKPGASVKVSCKVSGSTFTDHTIH |
| | | WMRQAPGQGLEWMGYIYPTSGSTQYAQKFQGRV |
| | | TLTADRSTNTAYMELSSLRSEDTAVYFCSLRPLFNV |
| | | IKHWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKKVEPKSCDKTHTCPPCPAPELLGGPSVFLFPPK |
| | | PKDTLMISRTPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 138 | LC of Ab-AHZ | DIVMTQSPDSLAVSLGERATINCKSSQSLLDRGNHK |
| | | NFLTWYQQKPGQPPKLLIFWASTRETGVPDRFSGS |
| | | GSGIDFTLTISSLQAEDVAVYYCQNDYRYPLTFGQG |
| | | TKLEIKRTVAAPSVFIFPPSDEQLKSGTASVVCLLNN |
| | | FYPREAKVQWKVDNALQSGNSQESVTEQDSKDST |
| | | YSLSSTLTLSKADYEKHKVYACEVTHQGLSSPVTK |
| | | SFNRGEC |
| 139 | HC of Ab-A | QVQLQQSDAELVKPGVSVKISCKVSGSTFTDHTIH |
| | | WMKQRPEQGLEWIGYIYPTDGSTQYNEKFRGKAT |
| | | LTADRSSNTAYMQLNSLTYEDSAVYFSSLRPLFNVI |
| | | KHWGQGTSVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKKVEPKSCDKTHTCPPCPAPELLGGPSVFLFPPK |
| | | PKDTLMISRTPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 140 | LC of Ab-A | DIVMTQSPSSLTVTAGEKVTMSCKSSQSLLDSGNH |
| | | KNFLTWYQQKPGQPPKLLIFWASTRETGVPDRFTG |
| | | SGSGIDFTLTITSVQAEDLAVYYCQNDYRYPLTFGA |
| | | GTKLELKRTVAAPSVFIFPPSDEQLKSGTASVVCLL |
| | | NNFYPREAKVQWKVDNALQSGNSQESVTEQDSKD |
| | | STYSLSSTLTLSKADYEKHKVYACEVTHQGLSSPV |
| | | TKSFNRGEC |
| 141 | Ab-BHZm heavy chain | XVQLVQSGAEVKKPGASVKVSCKASGFTFTSYWM |
| | amino acid sequence (N- | HWVRQAPGQGLEWIGNIQLSYGNTNYAQKFQGRV |
| | terminal | TLTVDKSSSTAYMELSSLRSEDTAVYYCARGFSYF |
| | pyroglutamylation) | DYWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKKVEPKSCDKTHTCPPCPAPEAAGAPSVFLFPPK |
| | | PKDTLMISRTPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| | | X is pyroglutamic acid or pyroglutamate |
| 142 | Ab-BHZm heavy chain | XVQLVQSGAEVKKPGASVKVSCKASGFTFTSYWM |
| | amino acid sequence (N- | HWVRQAPGQGLEWIGNIQLSYGNTNYAQKFQGRV |
| | terminal | TLTVDKSSSTAYMELSSLRSEDTAVYYCARGFSYF |
| | pyroglutamylation; | DYWGQGTLVTVSSASTKGPSVFPLAPSSKSTSGGT |
| | deletion of C-terminal | AALGCLVKDYFPEPVTVSWNSGALTSGVHTFPAVL |
| | lysine) | QSSGLYSLSSVVTVPSSSLGTQTYICNVNHKPSNTK |
| | | VDKKVEPKSCDKTHTCPPCPAPEAAGAPSVFLFPPK |
| | | PKDTLMISRTPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPG |
| | | X is pyroglutamic acid or pyroglutamate |
| 143 | Ab-BHZm heavy chain | QVQLVQSGAEVKKPGASVKVSCKASGFTFTSYWM |
| | amino acid sequence | HWVRQAPGQGLEWIGNIQLSYGNTNYAQKFQGRV |
| | (deletion of C-terminal | TLTVDKSSSTAYMELSSLRSEDTAVYYCARGFSYF |
| | lysine) | DYWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKKVEPKSCDKTHTCPPCPAPEAAGAPSVFLFPPK |
| | | PKDTLMISRTPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPG |
| 144 | Lambda CL | GQPKAAPSVTLFPPSSEELQANKATLVCLISDFYPG |
| | | AVTVAWKADSSPVKAGVETTTPSKQSNNKYAASS |
| | | YLSLTPEQWKSHRSYSCQVTHEGSTVEKTVAPTEC |
| | | S |
| 145 | Short chain polypeptide | GGFG |
| 146 | Short chain polypeptide | GGVA |
| 147 | Short chain polypeptide | GFLG |
| 148 | Short chain polypeptide | EAAA |
| 149 | Short chain polypeptide | GGGGG |
| 150 | Linker subunit | GGGGS |
| 151 | (GGGGS)₄ | GGGGSGGGGSGGGGSGGGGS |

The abbreviations used herein have the following meanings:

| Abbrevi- | | Abbrevi- | |
|---|---|---|---|
| ation | Meaning | ation | Meaning |
| HATU | N,N,N′,N′-tetramethyl-O-(7- | HOBt | 1-hydroxybenzotriazole |
| | azabenzotriazol-1-yl)uronium | | |
| | hexafluorophosphate | | |
| DIPEA | diisopropylethyl amine | EDCI | 1-(3-dimethylaminopropyl)- |
| | | | 3-ethylcarbodiimide |
| | | | hydrochloride |
| TFA | Trifluoroacetic Acid | DMF | N,N-dimethylformamide |
| Pd/C | Palladium on carbon | DMTMM | 4-(4,6-dimethoxytriazin-2- |
| | | | yl)-4-methylmorpholinium |
| | | | chloride |
| NBS | N-bromosuccinimide | IPA | Isopropanol |
| NaIO₄ | Sodium periodate | RuCl₃•H₂O | Ruthenium trichloride |
| | | | hydrate |
| XPhos Pd | Methanesulfonato(2- | LiOH•H₂O | Lithium hydroxide hydrate |
| G3 | dicyclohexylphosphino-2′,4′,6′- | | |
| | tri-isopropyl-1,1′-biphenyl)(2′- | | |
| | amino-1,1′-biphenyl-2- | | |
| | yl)palladium(II) | | |
| K₃PO₄ | Potassium phosphate | m-CPBA | m-chloroperoxybenzoic acid |
| THF | Tetrahydrofuran | DMSO | Dimethylsulfoxide |
| DCM | Dichloromethane | MeOH | Methanol |
| HPLC | High performance liquid | LC-MS | Liquid chromatography-mass |
| | chromatography | | spectrometry |
| TCEP | Tris(2-carboxyethyl)phosphine | Na₂HPO₄ | Disodium hydrogen |
| | | | phosphate |
| EDTA | Ethylenediaminetetraacetic acid | PB | Phosphate buffer |
| DAR | Drug loading ratio | | |

### EXAMPLES

The disclosure will be further described below through the description of specific embodiments, but this is not intended to limit the disclosure. Those skilled in the art can make various modifications or improvements based on the teachings of the disclosure without departing from the basic idea and scope of the disclosure.

The structures of the compounds described in the following examples were determined by nuclear magnetic resonance (¹H NMR) or mass spectrometry (MS).

Nuclear magnetic resonance (¹H NMR) measurements were performed using a Bruker 400 MHz NMR instrument; the deuterated reagent was hexadeuterated dimethyl sulfoxide (DMSO-d6); and the internal standard substance was tetramethylsilane (TMS).

The abbreviations in the nuclear magnetic resonance (NMR) spectra used in the examples are shown below.
s: Single peak (singlet), d: doublet, t: triplet, q: quartet, m: multiplet, br: broad, J: conjugation constant, Hz: Hertz, and DMSO-d6: deuterated dimethyl sulfoxide. Delta (δ) values are expressed in ppm.

The determination of mass spectrometry (MS) was determined using an Agilent (ESI) mass spectrometer with the model of Agilent 6120B (Agilent Technologies, Santa Clara, CA).

SMCC-DM1, which is commercially available or can be prepared with reference to US20100129314 A1, was used in the following examples and has the following structure:

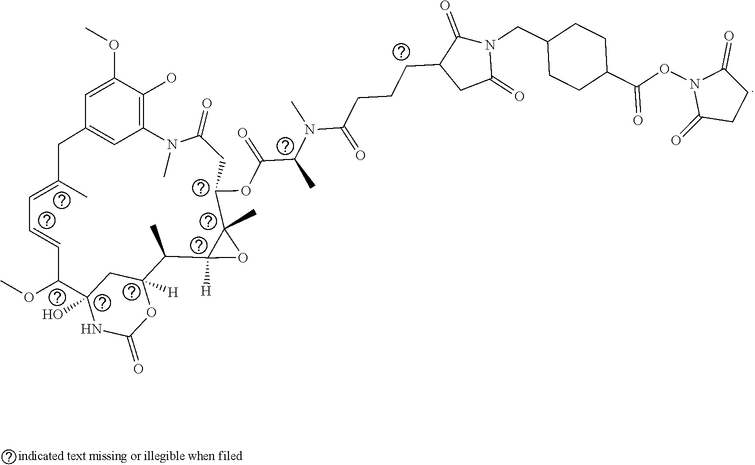 [SMILES: COc1cc2cc(c1O)N(C)C(=O)C[C@H](OC(=O)[C@H](C)N(C)C(=O)CCCC1CC(=O)N(CC3CCC(C(=O)ON4C(=O)CCC4=O)CC3)C1=O)[C@]1(C)O[C@H]1[C@H](C)[C@@H]1C[C@@](O)(NC(=O)O1)C(OC)/C=C/C=C(\C)C2]

### Example 1. Preparation of Compounds

### 1.1 Preparation of N—((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizine[1,2-b]quinolin-1-yl)amino)-1,6,9,12,15-pentaoxo-3-oxy-5,8,11,14-tetraazahexadecan-16-yl)-6-(2-(methylsulfonyl)pyrimidin-5-yl)hexan-5-amide (A-05)

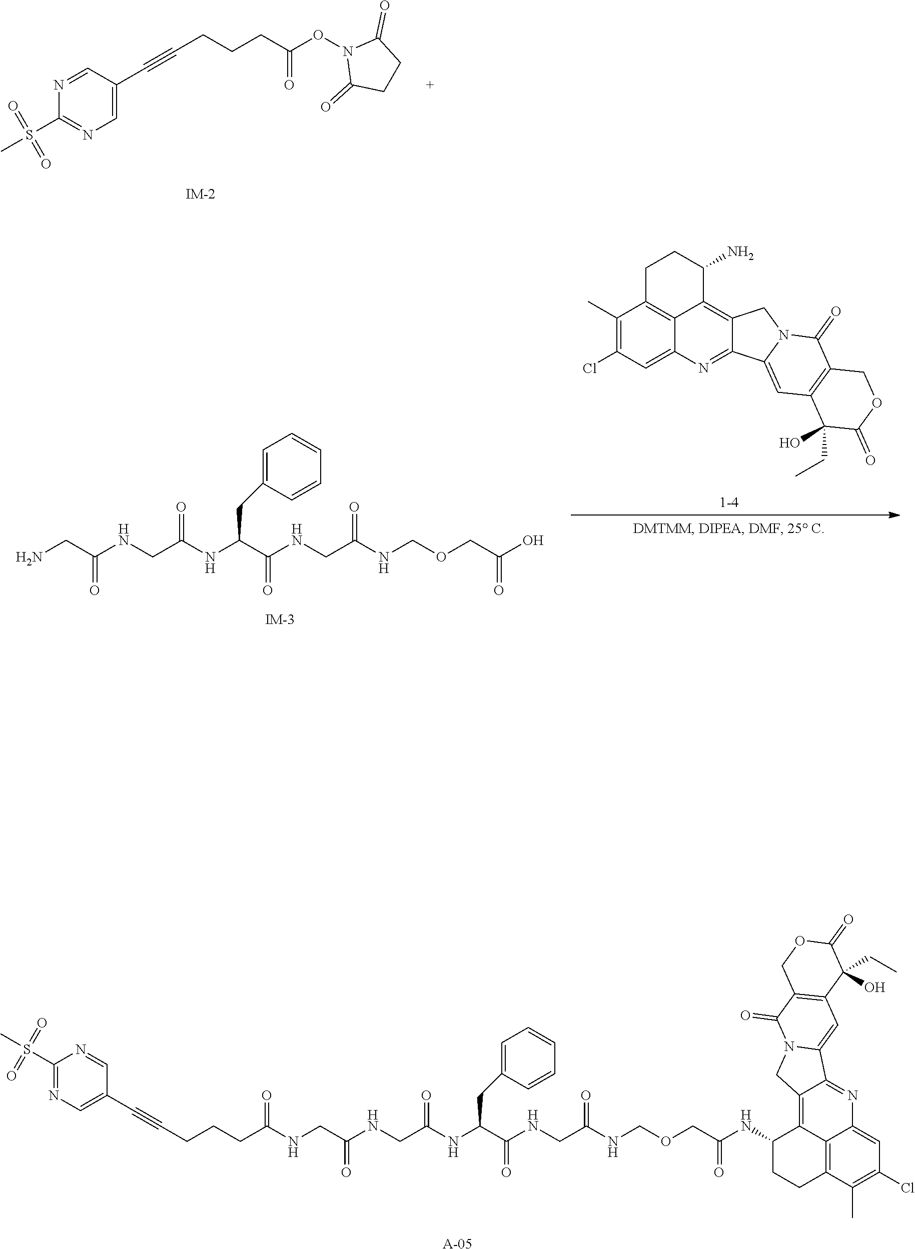

Under nitrogen protection, 2,5-dioxopyrrolidin-1-yl-6-(2-(methylsulfonyl)pyrimidin-5-yl)hexyl-5-ynoate (IM-2, 0.66 g, 1.80 mmol) and (R)-16-amino-10-benzyl-6,9,12,15-tetraoxo-3-oxo-5,8,11,14-tetraazahexadecanoic acid (IM-3, 0.75 g, 1.77 mmol) were added to DMF (19 mL); after the temperature was raised to 35° C. for a reaction for 16 hours, to the system was added (1S,9S)-1-amino-5-chloro-9-ethyl-9-hydroxy-4-methyl-1,2,3,9,12,15-hexahydro-10H,13H-benzo[de]pyrano[3′,4′:6,7]indolizine[1,2-b]quinoline-10,13-dione (1-4, 1.00 g, 1.77 mmol), and the system was cooled with ice water to 5-15° C., followed by an addition of DMTMM (0.98 g, 3.53 mmol) and then a dropwise addition of DIPEA (1.14 g, 8.84 mmol); and a reaction was performed at 25° C. for 16 hours. A reaction solution was poured into a mixture solution of DCM (600 mL), IPA (60 mL) and water (100 mL) and stirred for 10 minutes, and a DCM phase was separated, washed with brine (100 ml) and concentrated to afford a crude product, which was purified by preparative HPLC and then freeze-dried to afford 0.98 g of Compound A-05.

The separation and purification method of A-05 was as follows:

Chromatography column: Waters SunFire Prep C18 OBD (5 μm*19 mm*150 mm)

Mobile phase A: acetonitrile; Mobile phase B: Water (0.05% formic acid)

| Time | Mobile phase A | Mobile phase B | Flow rate |
|---|---|---|---|
| [min] | [%] | [%] | [mL/min] |
| 0.00 | 30 | 70 | 24 |
| 2.00 | 30 | 70 | 24 |
| 18.00 | 80 | 20 | 24 |

The structural characterization data of A-05 was as follows:

MS m/z (ESI): 1107.3 [M+H]+

¹H NMR (400 MHz, DMSO) δ 9.10 (s, 2H), 8.66-8.63 (m, 1H), 8.51 (d, J=8.8 Hz, 1H), 8.34-8.31 (m, 1H), 8.21-8.19 (m, 1H), 8.17-8.09 (m, 2H), 8.08-8.04 (m, 1H), 7.30 (s, 1H), 7.26-7.15 (m, 5H), 6.55 (s, 1H), 5.56-5.55 (m, 1H), 5.48-5.35 (m, 2H), 5.25-5.10 (m, 2H), 4.64 (d, J=6.4 Hz, 2H), 4.45-4.44 (m, 1H), 4.06-3.98 (m, 2H), 3.77-3.52 (m, 6H), 3.41 (s, 3H), 3.25-3.12 (m, 2H), 3.03-3.00 (m, 1H), 2.83-2.72 (m, 1H), 2.58-2.56 (m, 2H), 2.48 (s, 3H), 2.33-2.30 (m, 2H), 2.21-2.13 (m, 2H), 1.91-1.76 (m, 4H), 0.87 (t, J=7.2 Hz, 3H).

### 1.2 Preparation of N—((S)-10-benzyl-1-(((1S,9S)-5-fluoro-9-ethyl-9-hydroxy-4-chloro-10,13-dioxo-2,3,9,10,13,15-hexahydro-1H,12H-benzo[de]pyrano[3′,4′:6,7]indolizine[1,2-b]quinolin-1-yl)amino)-1,6,9,12,15-pentaoxo-3-oxy-5,8,11,14-tetraazahexadecan-16-yl)-6-(2-(methylsulfonyl)pyrimidin-5-yl)hexan-5-amide (A-07)

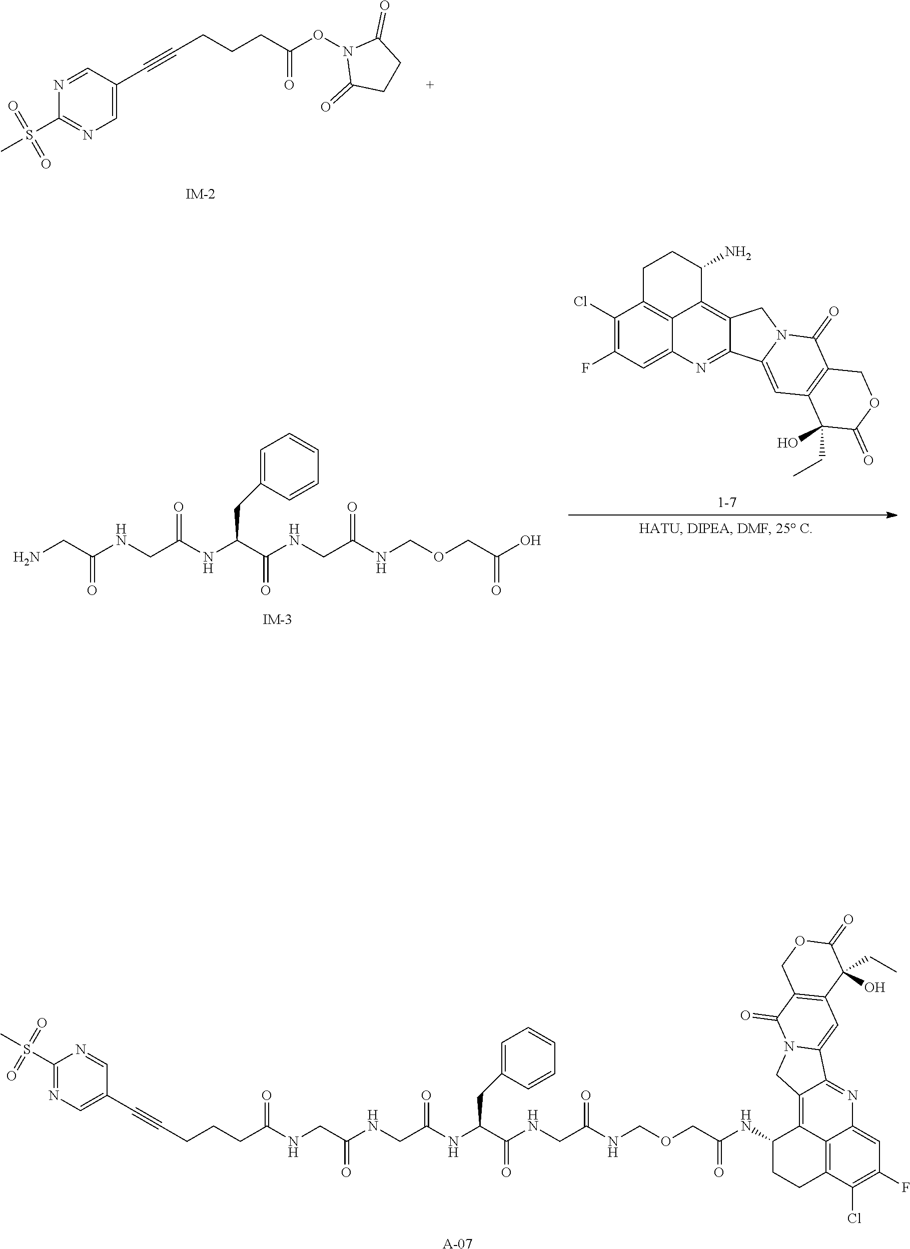

Under nitrogen protection, 2,5-dioxopyrrolidin-1-yl-6-(2-(methylsulfonyl)pyrimidin-5-yl)hexyl-5-ynoate (IM-2, 21.6 mg, 0.059 mmol) and (R)-16-amino-10-benzyl-6,9,12,15-tetraoxo-3-oxo-5,8,11,14-tetraazahexadecanoic acid (IM-3, 24.5 mg, 0.058 mmol) were added to DMF (1 mL); and after the temperature was raised to 35° C. for a reaction for 16 hours, to the system was added (1S,9S)-1-amino-5-fluoro-9-ethyl-9-hydroxy-4-chloro-1,2,3,9,12,15-hexahydro-10H,13H-benzo[de]pyrano[3′,4′:6,7]indolizine[1,2-b]quinoline-10,13-dione trifluoroacetate (30.0 mg, 0.053 mmol), HATU (30 mg, 0.079 mmol) and DIPEA (27.2 mg, 0.21 mmol), and a reaction system was reacted at 25° C. for 16 hours. A reaction solution was directly purified by preparative HPLC and then freeze-dried to afford 26.4 mg of Compound A-07.

The separation and purification method of A-07 was as follows:

Chromatography column: SunFire Prep C18 OBD 19 mm×150 mm×5.0 μm

Mobile phase A: acetonitrile; Mobile phase B: Water (0.05% formic acid)

| Time | Mobile phase A | Mobile phase B | Flow rate |
|---|---|---|---|
| [min] | [%] | [%] | [mL/min] |
| 0 | 30 | 70 | 28 |
| 2 | 30 | 70 | 28 |
| 18 | 90 | 10 | 28 |

The structural characterization data of A-07 was as follows:

ESI-MS (m/z):1111.3[M+H]+.

### 1.3 Preparation of N-((7S,10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizine[1,2-b]quinolin-1-yl)amino)-7,10-dimethyl-1,6,9,12-tetraoxo-3-oxo-5,8,11-triazatetradecan-13-yl)-6-(2-(methylsulfonyl)pyrimidin-5-yl)hexan-5-amide (A-14)

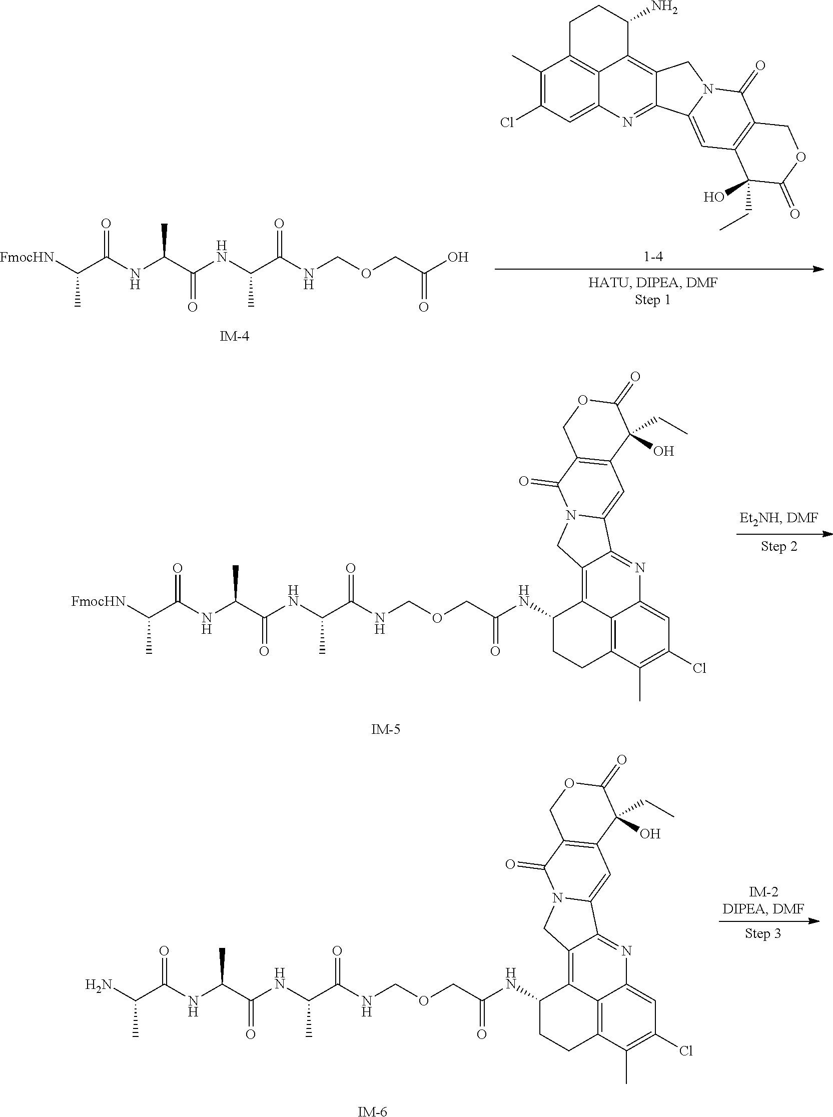

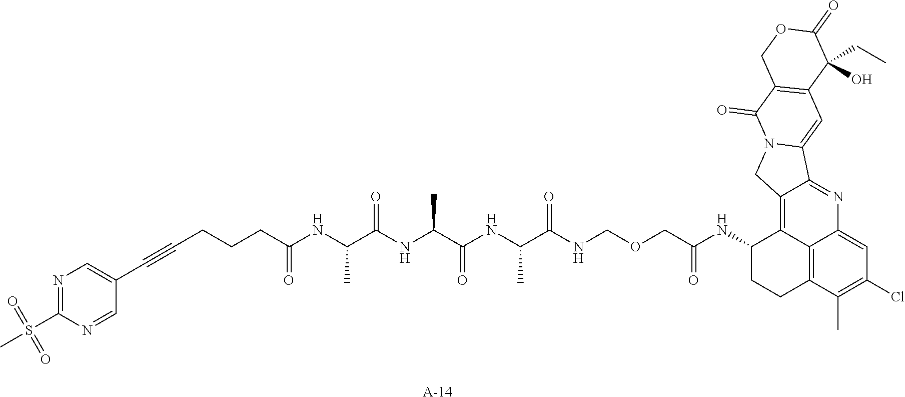 [SMILES: CC[C@@]1(O)C(=O)OCc2c1cc1n(c2=O)Cc2c-1nc1cc(Cl)c(C)c3c1c2[C@@H](NC(=O)COCNC(=O)[C@H](C)NC(=O)[C@H](C)NC(=O)[C@H](C)NC(=O)CCCC#Cc1cnc(S(C)(=O)=O)nc1)CC3 ; "A-14"]

### Step 1:

Compound IM-4 (657 mg, 1.22 mmol) and Compound 1-4 (500 mg, 1.11 mmol) were dissolved in N,N-dimethylformamide (10 mL), followed by an addition of HATU (630.67 mg, 1.66 mmol) and N,N-diisopropylethylamine (428 mg, 3.32 mmol), and a mixture was stirred at room temperature for 1 hour. After a reaction was completed, a reaction solution was directly purified by preparative HPLC and then freeze-dried to afford 700 mg of Compound IM-5.

A preparation method thereof was as follows:

Chromatography column: Waters SunFire Prep C18 OBD (5 μm*19 mm*150 mm)

Mobile phase A: acetonitrile; Mobile phase B: Water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 18.00 | 90 | 10 | 28 |

### Step 2:

Compound IM-5 (500 mg, 0.513 mmol) was dissolved in N,N-dimethylformamide (2 mL), diethylamine (75.05 mg, 1.03 mmol) was added, and a reaction was performed at room temperature for 1 hour. After the reaction was completed, a reaction solution was directly purified by preparative HPLC and then freeze-dried to afford 307 mg of Compound IM-6.

A preparation method thereof was as follows:

Chromatography column: Waters SunFire Prep C18 OBD (5 μm*19 mm*150 mm)

Mobile phase A: acetonitrile; Mobile phase B: Water (0.05% formic acid)

| Time | Mobile phase A | Mobile phase B | Flow rate |
|---|---|---|---|
| [min] | [%] | [%] | [mL/min] |
| 0.00 | 20 | 80 | 24 |
| 2.00 | 20 | 80 | 24 |
| 18.00 | 80 | 20 | 24 |

### Step 3:

IM-6 (170 mg, 0.226 mmol) and Compound IM-2 (90.83 mg, 0.249 mmol) were dissolved in N,N-dimethylformamide (10 mL), and N,N-diisopropylethylamine (29.21 mg, 0.226 mmol) was added. A reaction solution was stirred at room temperature for 16 hours. A reaction solution was directly purified by preparative HPLC and then freeze-dried to afford 50.56 mg of Compound A-14.

The structural characterization data was as follows:

MS m/z (ESI): 1002.4 [M+H]+

A preparation method thereof was as follows:

Chromatography column: Waters SunFire Prep C18 OBD (5 μm*19 mm*150 mm)

Mobile phase A: acetonitrile; Mobile phase B: Water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 18.00 | 90 | 10 | 28 |

¹H NMR (400 MHz, DMSO) δ 9.11 (s, 2H), 8.68 (t, J=6.4 Hz, 1H), 8.49 (d, J=8.8 Hz, 1H), 8.16 (s, 1H), 8.10 (d, J=7.2 Hz, 1H), 8.01 (d, J=7.2 Hz, 1H), 7.91 (d, J=6.8 Hz, 1H), 7.3 1 (s, 1H), 6.55 (s, 1H), 5.65-5.55 (m, 1H), 5.43 (s, 2H), 5.21 (s, 2H), 4.67-4.55 (m, 2H), 4.29-4.15 (m, 3H), 3.98 (s, 2H), 3.41 (s, 3H), 3.25-3.15 (m, 2H), 2.57-2.56 (m, 2H), 2.35-2.27 (m, 2H), 2.22-2.12 (m, 2H), 1.91-1.75 (m, 4H), 1.23-1.09 (m, 9H), 0.87 (t, J=7.2 Hz, 3H).

### 1.4 Synthesis of 4-((S)-2-(4-aminobutyl)-35-(4-((6-(2-(methylsulfonyl)pyrimidin-5-yl)hex-5-ynamido)methyl)-1H-1,2,3-triazol-1-yl)-4,8-dioxo-6,12,15,18,21,24,27,30,33-nonaoxa-3,9-diazapentatriacontamido)benzyl((S)-4-ethyl-11-(2-(N-isopropylmethylsulfonamido)ethyl)-3,14-dioxo-3,4,12,14-tetrahydro-1H-pyrano[3′,4′:6,7]indolizino[1,2-b]quinolin-4-yl)carbonate (B-01)

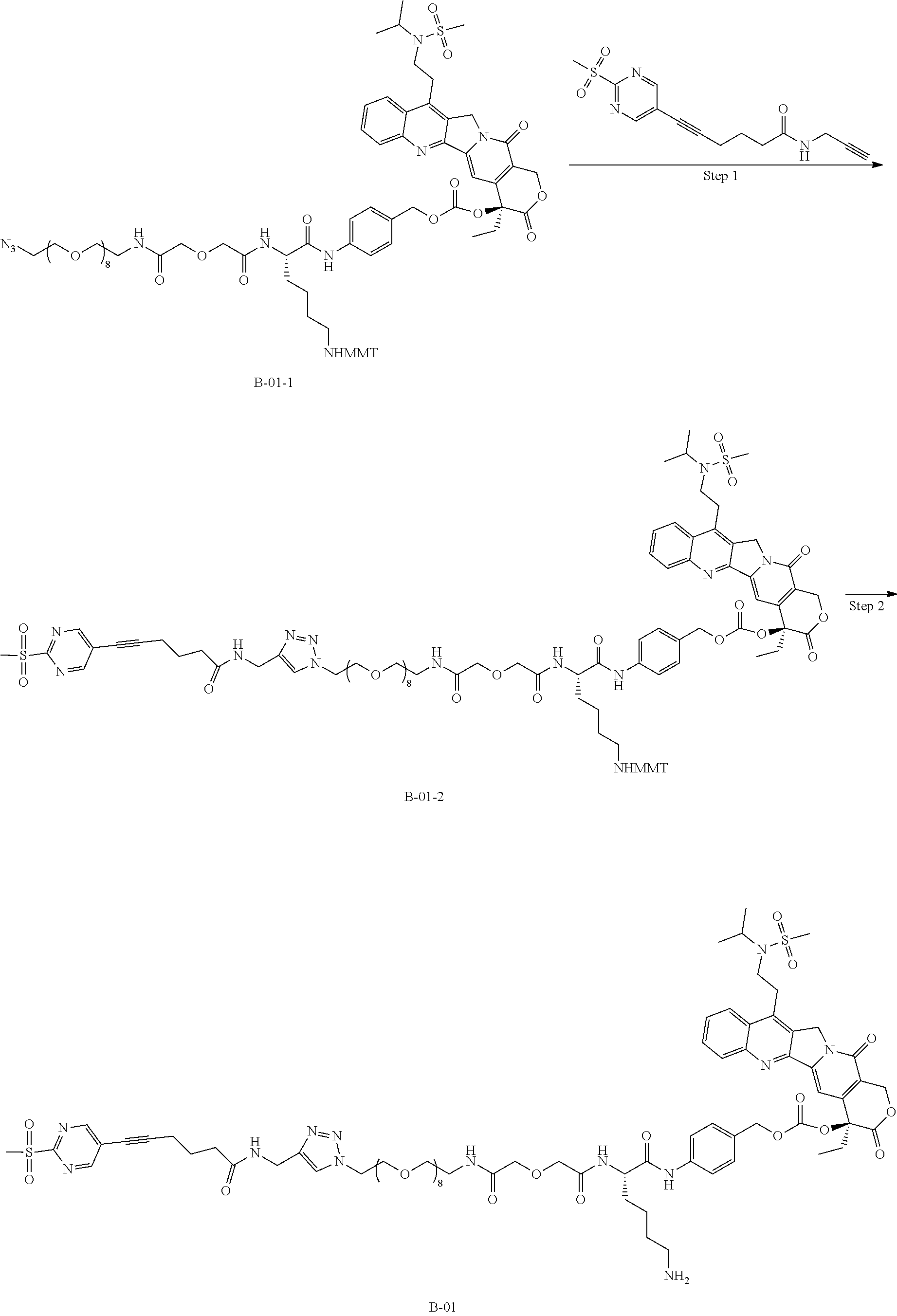

### Step 1

At room temperature, Compound B-01-1 (413.40 mg, 0.251 mmol; refer to patent CN111295389B for synthesis thereof) was dissolved in dimethyl sulfoxide and water (2.0 mL:0.5 mL), and cuprous bromide (72.95 mg, 0.503 mmol) and 6-(2-(methylsulfonyl)pyrimidin-5-yl)-N-(prop-2-yn-1-yl)-hex-5-ynamide (95.10 mg, 0.302 mmol) were added; and filtering was performed after stirring and reacting for 1 h, and a filtrate was purified by preparative high performance liquid chromatography (conditions as follows) to afford 30.00 mg of Compound B-01-2.

Chromatography column: SunFire Prep C 18 OBD 19 mm×150 mm×5.0 μm

Mobile phase A: acetonitrile; Mobile phase B: water

| Time | Mobile phase A | Mobile phase B | Flow rate |
|---|---|---|---|
| [min] | [%] | [%] | [mL/min] |
| 0.00 | 40 | 60 | 28 |
| 8.00 | 40 | 60 | 28 |
| 50.00 | 90 | 10 | 28 |

### Step 2:

Compound B-01-2 (30.00 mg, 0.02 mmol) was dissolved in dichloromethane (1.0 mL), trifluoroacetic acid (0.2 mL) was added to a reaction solution, and a reaction was performed at room temperature for 30 min. A reaction solution was concentrated under reduced pressure and then purified by preparative HPLC (conditions as follows) to afford 20.00 mg of trifluoroacetate of Compound B-01.

Chromatography column: SunFire Prep C18 OBD 19 mm×150 mm×5.0 μm

Mobile phase A: acetonitrile; Mobile phase B: water (0.05% trifluoroacetic acid)

| Time | Mobile phase A | Mobile phase B | Flow rate |
|---|---|---|---|
| [min] | [%] | [%] | [mL/min] |
| 0.00 | 15 | 85 | 28 |
| 8.00 | 15 | 85 | 28 |
| 50.00 | 60 | 40 | 28 |

The structural characterization data was as follows: ESI-MS (m/z):1631.7[M+H]⁺, 816.0[M/2+H]⁺.

### Example 2. Preparation of Antibodies

### 2.1 Immunization of Mice

The extracellular sequences of human CDH3 (NP_001304124.1), monkey CDH3 (XP_005592414.1), rat CDH3 (XP_006255621.1) and mouse CDH3 (NP_001032898.1), each having a 6×His tag added to the C-terminal, were optimized, synthesized, and constructed onto a pcDNA3.4 expression vector; and the recombinant plasmid was extracted, and transfected, expressed and purified to obtain human, monkey, rat and mouse CDH3 proteins after being sequenced to be free of errors. The human CDH3 full-length gene was used to construct the BaF3 (Signosis, Cat #EL-001) overexpression cell BaF3-hCDH3. Wild-type mice were immunized with human CDH3 proteins, and Freund's complete adjuvant and Freund's incomplete adjuvant were used for immunization, respectively. The serum titer of an anti-CDH3 antibody was monitored by BaF3 overexpression cell flow cytometry. According to the serum titer, the best mice were selected for hybridoma fusion.

According to the NOV169N31Q sequence of WO2016203432A1, a positive control antibody PCA062 mAb was prepared.

To prepare a negative control antibody, a variable region of a mouse anti-chicken lysozyme antibody HyHEL-10 was fused with a human IgG1 heavy chain constant region comprising the amino acid sequence set forth in SEQ ID NO: 121 and a human Kappa light chain constant region comprising the amino acid sequence set forth in SEQ ID NO: 123, and the expressed and purified antibody was named hIgG1. The variable region of mouse anti-chicken lysozyme antibody HyHEL-10 was fused with a human IgG1 heavy chain constant region mutant comprising the amino acid sequence set forth in SEQ ID NO: 122 and a human Kappa light chain constant region comprising the amino acid sequence set forth in SEQ ID NO: 123, and the expressed and purified antibody was named hIgG1m.

### 2.2 Hybridoma Fusion Screening

After hybridoma fusion, BaF3-hCDH3 cell flow cytometry was used for supernatant screening to initially screen a positive clone supernatant, and then ELISA was used to detect the clones positive for binding to rat CDH3 proteins and not binding to blank BaF3 cells for further verification. Candidate clones were expanded to a 24-well plate and subcloned, and then monoclonals were screened by using BaF3-hCDH3 and blank BaF3 cells; and the clones positive for binding to BaF3-hCDH3 cells and not binding to blank BaF3 cells were selected for amplification and culture.

### 2.3 Anti-Human CDH3 Murine Antibody

All preferred monoclonals were amplified for serum-free culture, and Protein-A resin (MabSelect SuRe, Cytiva) was used to purify a supernatant by affinity, to obtain CDH3 murine antibodies Ab-B, Ab-C, Ab-D, and Ab-A after quantification.

### 2.4 Humanization and Expression of Anti-Human CDH3 Antibodies

Murine antibodies Ab-C, Ab-B, Ab-D, and Ab-A were subjected to humanization modification by a CDR transplantation antibody humanization modification method. In short, the humanization modification involves the following steps of: comparing an amino acid sequence of a murine monoclonal antibody with an amino acid sequence of a human germline antibody to find the sequence with high homology and better physical and chemical properties as a human germline framework sequence; analyzing and examining the HLA-DR affinity, and selecting the human germline framework sequence with a low affinity; and then transplanting six CDRs of the murine antibody to the selected heavy chain and light chain framework sequences, respectively.

Further by a computer simulation technology, molecular docking was applied to analyze the variable region and a surrounding framework amino acid sequence thereof, so as to examine the spatial stereoscopic binding mode thereof. By calculating an electrostatic force, a van der Waals force, hydrophilicity and an entropy value, the key amino acids in the amino acid sequence of the murine antibody that can interact with CDH3 proteins and maintain the spatial structure were analyzed, and these murine amino acids were retained in the transplanted antibody. That is, a series of back mutations were performed on the amino acid residues in the FR region of the above humanized template, so that the humanized antibody could retain the antigen-binding ability of the murine antibody as much as possible.

According to the above method, a plurality of humanized antibodies was constructed on the basis of the CDRs of murine antibodies Ab-B, Ab-C, Ab-D, and Ab-A. By the detections of the protein and cell binding, expression level, thermal stability, hydrophobicity, freeze-thaw stability and the like, the final humanized molecules were named Ab-BHZ, Ab-CHZ, Ab-DHZ, and Ab-AHZ, respectively; wherein the heavy chain constant regions of Ab-BHZ, Ab-CHZ, Ab-DHZ, and Ab-AHZ antibodies were all human IgG1 heavy chain constant regions (SEQ ID NO: 121); and the light chain constant regions of Ab-BHZ, Ab-DHZ, and Ab-AHZ antibodies were all human Kappa light chain constant regions (SEQ ID NO: 123), and the light chain constant region of the Ab-CHZ antibody was a human lambda light chain constant region (SEQ ID NO: 144). In addition, according to the above method, the human IgG1 heavy chain constant region of Ab-BHZ (SEQ ID NO: 121) was replaced with a mutant human heavy chain constant region IgG1m (SEQ ID NO: 122), and the expressed antibody was named Ab-BHZm.

The humanized antibody was entrusted to Nanjing GenScript Biotechnology Co., Ltd. for codon optimization as well as synthesis and ligation into the expression plasmid pTT5. The heavy chain and light chain expression plasmids of the humanized antibody were transfected into CHO-S cells at the same time. After expression for 7 days, a supernatant was collected by centrifugation. A recombinant antibody in the supernatant was purified by Protein A (MabSelect SuRe, Cytiva) to afford an anti-human CDH3 humanized antibody.

The variable regions and CDR sequences of the anti-human CDH3 murine antibody and the humanized antibody obtained above are summarized in the following table.

TABLE 1
Sequence information on Ab-BHZ, Ab-AHZ, and Ab-BHZm (SEQ ID NO:)

| | SEQ ID NO: | | | SEQ ID NO: | | | | | |
|---|---|---|---|---|---|---|---|---|---|
| | Heavy chain | Light chain | | Heavy | Heavy | Heavy | Light | Light | Light |
| | variable | variable | Definition | chain | chain | chain | chain | chain | chain |
| Antibody | region | region | mode | CDR1 | CDR2 | CDR3 | CDR1 | CDR2 | CDR3 |
| Ab-BHZm/ | 3 | 4 | Chothia | 17 | 27 | 32 | 35 | 38 | 42 |
| Ab-BHZ | | | AbM | 18 | 28 | 32 | 35 | 38 | 42 |
| | | | Kabat | 19 | 29 | 32 | 35 | 38 | 42 |
| | | | Contact | 20 | 30 | 33 | 36 | 40 | 43 |
| | | | IMGT | 21 | 31 | 34 | 37 | 41 | 42 |
| Ab-B | 1 | 2 | Chothia | 17 | 22 | 32 | 35 | 38 | 42 |
| | | | AbM | 18 | 23 | 32 | 35 | 38 | 42 |
| | | | Kabat | 19 | 24 | 32 | 35 | 38 | 42 |
| | | | Contact | 20 | 25 | 33 | 36 | 39 | 43 |
| | | | IMGT | 21 | 26 | 34 | 37 | 41 | 42 |
| Ab-CHZ | 7 | 8 | Chothia | 44 | 49 | 55 | 58 | 63 | 66 |
| | | | AbM | 45 | 50 | 55 | 58 | 63 | 66 |
| | | | Kabat | 46 | 52 | 55 | 58 | 63 | 66 |
| | | | Contact | 47 | 53 | 56 | 59 | 64 | 67 |
| | | | IMGT | 48 | 54 | 57 | 60 | 65 | 66 |
| Ab-C | 5 | 6 | Chothia | 44 | 49 | 55 | 58 | 61 | 66 |
| | | | AbM | 45 | 50 | 55 | 58 | 61 | 66 |
| | | | Kabat | 46 | 51 | 55 | 58 | 61 | 66 |
| | | | Contact | 47 | 53 | 56 | 59 | 62 | 67 |
| | | | IMGT | 48 | 54 | 57 | 60 | 65 | 66 |
| Ab-DHZ | 11 | 12 | Chothia | 68 | 73 | 80 | 84 | 88 | 91 |
| | | | AbM | 69 | 74 | 80 | 84 | 88 | 91 |
| | | | Kabat | 70 | 77 | 80 | 84 | 88 | 91 |
| | | | Contact | 71 | 78 | 81 | 85 | 89 | 92 |
| | | | IMGT | 72 | 79 | 82 | 86 | 90 | 91 |
| Ab-D | 9 | 10 | Chothia | 68 | 73 | 80 | 83 | 87 | 91 |
| | | | AbM | 69 | 74 | 80 | 83 | 87 | 91 |
| | | | Kabat | 70 | 75 | 80 | 83 | 87 | 91 |
| | | | Contact | 71 | 76 | 81 | 85 | 89 | 92 |
| | | | IMGT | 72 | 79 | 82 | 86 | 90 | 91 |
| Ab-AHZ | 15 | 16 | Chothia | 93 | 103 | 108 | 114 | 117 | 119 |
| | | | AbM | 94 | 104 | 108 | 114 | 117 | 119 |
| | | | Kabat | 95 | 105 | 108 | 114 | 117 | 119 |
| | | | Contact | 96 | 106 | 109 | 115 | 118 | 120 |
| | | | IMGT | 97 | 107 | 110 | 116 | 41 | 119 |
| Ab-A | 13 | 14 | Chothia | 93 | 98 | 108 | 111 | 117 | 119 |
| | | | AbM | 94 | 99 | 108 | 111 | 117 | 119 |
| | | | Kabat | 95 | 100 | 108 | 111 | 117 | 119 |
| | | | Contact | 96 | 101 | 109 | 112 | 118 | 120 |
| | | | IMGT | 97 | 102 | 110 | 113 | 41 | 119 |

### Example 3. Antibody Conjugation and Detection

### 3.1 Preparation of Ab-BHZm-A-14 (DAR 8)

2.727 mL of Ab-BHZm antibody (22 mg/mL) was taken and diluted with 136 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 228 μL, pH 7.60) solution was added and mixed evenly, followed by standing at room temperature for 1.5 h. Then a solution of A-14 (445.83 L, 10 mM) dissolved in dimethyl sulfoxide in an amount 10 times the amount of the antibody was added and mixed evenly, followed by standing at room temperature for 2 h; and after completion, an NAP gel column (Cytiva) was used to replace a buffer with a 20 mM histidine buffer solution at pH 6.0 to afford an antibody-drug conjugate (i.e., ADC Ab-BHZm-A-14). The DAR value determined by mass spectrometry was 8.0.

### 3.2 Preparation of Ab-BHZm-A-05 (DAR 8)

1.364 mL of Ab-BHZm antibody (22 mg/mL) was taken and diluted with 68 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 114 μL, pH 7.60) solution was added and mixed evenly, followed by standing at room temperature for 1.5 h. Then a solution of A-05 (218.22 μL, 10 mM) dissolved in dimethyl sulfoxide in an amount 10 times the amount of the antibody was successively added and mixed evenly, followed by standing at room temperature for 2 h; and after completion, an NAP gel column (Cytiva) was used to replace a buffer with a 20 mM histidine buffer solution at pH 6.0 to afford an antibody-drug conjugate (i.e., ADC Ab-BHZm-A-05). The DAR value determined by mass spectrometry was 7.99.

### 3.3 Preparation of Ab-BHZm-B-01 (DAR 8)

1.364 mL of Ab-BHZm antibody (22 mg/mL) was taken and diluted with 68 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 114 μL, pH 7.60) solution was added and mixed evenly, followed by standing at room temperature for 1.5 h. Then a solution of B-01 (211.54 μL, 10 mM) dissolved in dimethyl sulfoxide in an amount 10 times the amount of the antibody was sequentially added and mixed evenly, followed by standing at room temperature for 2 h; and after completion, an NAP gel column (Cytiva) was used to replace a buffer with 20 mM histidine buffer solution at pH 6.0 to afford an antibody-drug conjugate (i.e., ADC Ab-BHZm-B-01). The DAR value determined by mass spectrometry was 7.98.

### 3.4 Preparation of Ab-BHZ-A-14 (DAR 8)

2.4 mL of Ab-BHZ antibody (12.5 mg/mL) was taken and diluted with 120 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 114 μL, pH 7.60) solution was added and mixed evenly, followed by standing at room temperature for 1.5 h. Then a solution of A-14 (211.54 μL, 10 mM) dissolved in dimethyl sulfoxide in an amount 10 times the amount of the antibody was sequentially added and mixed evenly, followed by standing at room temperature for 2 h; and after completion, an NAP gel column (Cytiva) was used to replace a buffer with a 20 mM histidine buffer solution at pH 6.0 to afford an antibody-drug conjugate (i.e., ADC Ab-BHZ-A-14). The DAR value determined by mass spectrometry was 8.02.

### 3.5 Preparation of Ab-BHZ-A-05 (DAR 8)

3.15 mL of Ab-BHZ antibody (9.1 mg/mL) was taken and diluted with 158 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 108 μL, pH 7.60) solution was added and mixed evenly, followed by standing at room temperature for 1.5 h. Then a solution of A-05 (197 μL, 10 mM) dissolved in dimethyl sulfoxide in an amount 10 times the amount of the antibody was added and mixed evenly, followed by standing at room temperature for 2 h; and after completion, an NAP gel column (Cytiva) was used to replace a buffer with a 20 mM histidine buffer solution at pH 6.0 to afford an antibody-drug conjugate (i.e., ADC Ab-BHZ-A-05). The DAR value determined by mass spectrometry was 7.95.

### 3.6 Preparation of Ab-BHZ-B-01 (DAR 8)

3.15 mL of Ab-BHZ antibody (9.1 mg/mL) was taken and diluted with 158 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 108 μL, pH 7.60) solution was added and mixed evenly, followed by standing at room temperature for 1.5 h. Then a solution of B-01 (197 μL, 10 mM) dissolved in dimethyl sulfoxide in an amount 10 times the amount of the antibody was added and mixed evenly, followed by standing at room temperature for 2 h; after completion, an NAP gel column (Cytiva) was used to replace a buffer with a 20 mM histidine buffer solution at pH 6.0 to afford an antibody-drug conjugate (i.e., ADC Ab-BHZ-B-01). The DAR value determined by mass spectrometry was 7.92.

### 3.7 Preparation of Ab-BHZm-DM1-2 (DAR 4)

2.4 mL of Ab-BHZm antibody (12.5 mg/mL) was taken and diluted with 120 μL of 20 mM PB+0.1 M EDTA (pH 7.60), and then pH was adjusted to 8.0 with a 1 M Na₂HPO₄ solution; then a solution of SMCC-DM1 (261.87 μL, 10 mM) dissolved in dimethyl sulfoxide in an amount 12 times the amount of the antibody was added and mixed evenly, followed by standing at room temperature for 18 h; and after completion, an NAP gel column (Cytiva) was used to replace a buffer with a 20 mM histidine buffer solution at pH 6.0 to afford an antibody-drug conjugate (i.e., ADC Ab-BHZm-DM1-2). The DAR value determined by mass spectrometry was 3.99.

### 3.8 Preparation of PCA062-A-14 (DAR 8)

3.881 mL of PCA062 antibody (7.73 mg/mL) was taken and diluted with 200 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 113 μL, pH 7.60) solution was added and mixed evenly, followed by standing at room temperature for 1.5 h. Then a solution of A-14 (216.3 L, 10 mM) dissolved in dimethyl sulfoxide in an amount 10 times the amount of the antibody was successively added and mixed evenly, followed by standing at room temperature for 2 h; and after completion, an NAP gel column (Cytiva) was used to replace a buffer with a 20 mM histidine buffer solution at pH 6.0 to afford an antibody-drug conjugate (i.e., ADC Ab-PCA062-A-14). The DAR value determined by mass spectrometry was 7.96.

3.9 Preparation of hIgG1m-A-14 (DAR 8)

4.098 mL of hIgG1m antibody (18.3 mg/mL) was taken and diluted with 205 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 286.2 μL, pH 7.60) solution was added and mixed evenly, followed by standing at room temperature for 1.5 h. Then a solution of A-14 (531 μL, 10 mM) dissolved in dimethyl sulfoxide in an amount 10 times the amount of the antibody was successively added and mixed evenly, followed by standing at room temperature for 2 h; and after completion, an NAP gel column (Cytiva) was used to replace a buffer with a 20 mM histidine buffer solution at pH 6.0 to afford an antibody-drug conjugate (i.e., ADC hIgG1m-A-14). The DAR value determined by mass spectrometry was 8.02.

3.10 Preparation of hIgG1m-A-05 (DAR 8)

0.44 mL of hIgG1m antibody (27.4 mg/mL) was taken and diluted with 21.9 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 45.7 μL, pH 7.60) solution was added and mixed evenly, followed by standing at room temperature for 1.5 h. Then a solution of A-05 (86.55 μL, 10 mM) dissolved in dimethyl sulfoxide in an amount 10 times the amount of the antibody was sequentially added and mixed evenly, followed by standing at room temperature for 2 h; and after completion, an NAP gel column (Cytiva) was used to replace a buffer with a 20 mM histidine buffer solution at pH 6.0 to afford an antibody-drug conjugate (i.e., ADC hIgG1m-A-05). The DAR value determined by mass spectrometry was 8.

3.11 Preparation of hIgG1m-B-01 (DAR 8)

4.098 mL of hIgG1m antibody (18.3 mg/mL) was taken and diluted with 205 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 286.2 μL, pH 7.60) solution was added and mixed evenly, followed by standing at room temperature for 1.5 h. Then a solution of B-01 (531 μL, 10 mM) dissolved in dimethyl sulfoxide in an amount 10 times the amount of the antibody was added and mixed evenly, followed by standing at room temperature for 2 h; after completion, an NAP gel column (Cytiva) was used to replace a buffer with a 20 mM histidine buffer solution at pH 6.0 to afford an antibody-drug conjugate (i.e., ADC hIgG1m-B-01). The DAR value determined by mass spectrometry was 7.96.

3.12 Preparation of hIgG1m-DM1 (DAR 4)

1.533 mL of hIgG1m antibody (19.57 mg/mL) was taken and diluted with 77 μL of 20 mM PB+0.1 M EDTA (pH 7.60), and then pH was adjusted to 8.0 with a 1 M Na₂HPO₄ solution; then a solution of SMCC-DM1 (262.92 μL, 10 mM) dissolved in dimethyl sulfoxide in an amount 12 times the amount of the antibody was added and mixed evenly, followed by standing at room temperature for 18 h; and after completion, an NAP gel column (Cytiva) was used to replace a buffer with a 20 mM histidine buffer solution at pH 6.0 to afford an antibody-drug conjugate (i.e., ADC hIgG1m-DM1). The DAR value determined by mass spectrometry was 4.25.

### 3.13 Preparation of Example PCA062-2 (DAR 4)

3.881 mL of PCA062 antibody (7.73 mg/mL) was taken and diluted with 194 μL of 20 mM PB+0.1 M EDTA (pH 7.60), and then pH was adjusted to 8.0 with a 1 M Na₂HPO₄ solution; then a solution of SMCC-DM1 (324.44 μL, 10 mM) dissolved in dimethyl sulfoxide in an amount 15 times the amount of the antibody was added and mixed evenly, followed by standing at room temperature for 18 h; and after completion, an NAP gel column (Cytiva) was used to replace a buffer with a 20 mM histidine buffer solution at pH 6.0 to afford an antibody-drug conjugate (i.e. ADC PCA062-2). The DAR value determined by mass spectrometry was 4.12.

### 3.14 Preparation of Ab-AHZ-A-05 (DAR 8)

1.6 mL of 17A5HZ antibody (12.5 mg/mL) was taken and diluted with 80 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 73.5 μL, pH 7.60) solution was added and mixed evenly, followed by standing at room temperature for 1.5 h. Then a solution of A-05 (137 μL, 10 mM) dissolved in dimethyl sulfoxide in an amount 10 times the amount of the antibody was added and mixed evenly, followed by standing at room temperature for 2 h; and after completion, an NAP gel column (Cytiva) was used to replace a buffer with a 20 mM histidine buffer solution at pH 6.0 to afford an antibody-drug conjugate (i.e., ADC Ab-AHZ-A-05). The DAR value determined by mass spectrometry was 8.01.

### 3.15 Preparation of Ab-AHZ-B-01 (DAR 8)

1.6 mL of 17A5HZ antibody (12.5 mg/mL) was taken and diluted with 80 μL of 20 mM PB+0.1 M EDTA (pH 7.60), and then pH was adjusted to 7.6 with a 1 M Na₂HPO₄ solution; and a 10 mM TCEP (tris(2-carboxyethyl)phosphine, 73.5 μL, pH 7.60) solution was added and mixed evenly, followed by standing at room temperature for 1.5 h. Then a solution of B-01 (137 μL, 10 mM) dissolved in dimethyl sulfoxide in an amount 10 times the amount of the antibody was added and mixed evenly, followed by standing at room temperature for 2 h; after completion, an NAP gel column (Cytiva) was used to replace a buffer with a 20 mM histidine buffer solution at pH 6.0 to afford an antibody-drug conjugate (i.e., ADC Ab-AHZ-B-01). The DAR value determined by mass spectrometry was 7.96.

### 3.16 Preparation of Ab-AHZ-DM1 (DAR 4)

1.6 mL of 17A5HZ antibody (12.5 mg/mL) was taken, pH was adjusted to 8 with a 1 M Na₂HPO₄ solution, and then a solution of SMCC-DM1 (123 μL, 10 mM) dissolved in dimethyl sulfoxide in an amount 9 times the amount of the antibody was added and mixed evenly, followed by standing at room temperature for 18 h; and after completion, an NAP gel column (Cytiva) was used to replace a buffer with a 20 mM histidine buffer solution at pH 6.0 to afford an antibody-drug conjugate (i.e. ADC Ab-AHZ-DM1). The DAR value determined by mass spectrometry was 3.44.

A drug/antibody ratio (DAR value) of a conjugated sample was determined as follows:

An ADC sample after conjugation was subjected to LC-MS molecular weight analysis.

### Chromatographic Assay Conditions:

Liquid chromatography column: Thermo MAbPac RP 3.0*100 mm;

Mobile phase A: 0.1% FA/H₂O; Mobile phase B: 0.1% FA/ACN;

Flow rate: 0.25 ml/min; Sample chamber temperature: 8° C.; Column temperature: 60° C.; Injection volume: 2 μl;

| Time (minute) | 2 | 20 | 22 | 25 | 26 | 30 |
|---|---|---|---|---|---|---|
| Mobile phase A (volume %) | 75 | 60 | 5 | 5 | 75 | 75 |
| Mobile phase B (volume %) | 25 | 40 | 95 | 95 | 25 | 25 |

### Mass Spectrum Assay Conditions:

Mass spectrometer model: AB Sciex Triple TOF 5600+;

GS1 35; GS2 35; CUR 30; TEM 350; ISVF 5500; DP 200; CE 10; Accumulation time 0.5 s; m/z 600-4000; Time bins to sum 40.

TABLE 2
ADC quality detection results

| Drug-linker | | Purity | |
|---|---|---|---|
| NO. | ADC No. | SEC % | DAR |
| A-05 | Ab-BHZ-A-05 | 97.7 | 7.95 |
| A-05 | Ab-BHZm-A-05 | 97.4 | 7.99 |
| A-05 | Ab-AHZ-A-05 | 97.5 | 8.01 |
| B-01 | Ab-BHZ-B-01 | 98.3 | 7.92 |
| B-01 | Ab-AHZ-B-01 | 98.0 | 7.96 |
| B-01 | Ab-BHZm-B-01 | 95.5 | 7.98 |
| SMCC-DM1 | Ab-BHZm-DM1-2 | 93.1 | 3.99 |
| SMCC-DM1 | Ab-AHZ-DM1 | 97.4 | 3.44 |
| SMCC-DM1 | PCA062-2 | 98.2 | 4.12 |
| A-14 | Ab-BHZ-A-14 | 97.8 | 8.02 |
| A-14 | Ab-BHZm-A-14 | 97 | 8.0 |
| A-14 | PCA062-A-14 | 96.2 | 7.96 |
| A-05 | hIgG1m-A-05 | 98.2 | 8 |
| A-14 | hIgG1m-A-14 | 98.7 | 8.02 |
| B-01 | hIgG1m-B-01 | 96.6 | 7.96 |
| SMCC-DM1 | hIgG1m-DM1 | 97.8 | 4.25 |

### Example 4. Detection of Binding and Activity

### 4.1 Detection of Cellular Affinity of Anti-Human CDH3 Antibodies and Conjugates Thereof

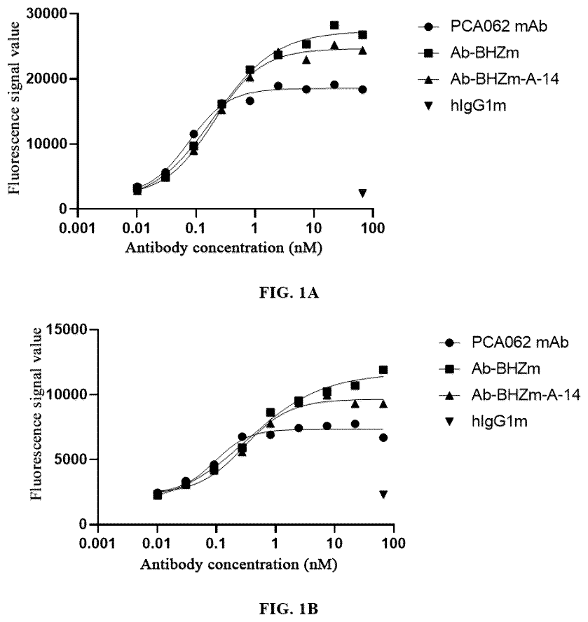

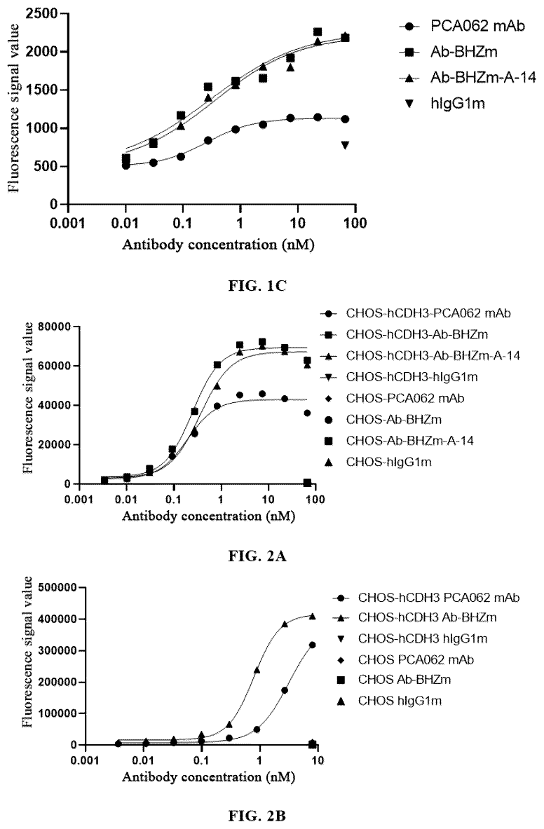

A flow cytometer (Beckman, model Cytoflex) was used to detect the affinity of an anti-human CDH3 antibody and a conjugated ADC with NCI-H358, HCC70, HCC1954, FADU, HCC1569, SK-CO-1, MKN45 (purchased from Nanjing Cobioer Bioscience Co., Ltd.), HCC1806, OVCAR3 and NCI-H146 (purchased from ATCC) cells. Adherently grown cells were digested with a cell dissociation buffer (Thermo), counting was performed, 2-3×10⁵ cells were taken from each well, incubated with the gradient-diluted antibody\ADC, washed, added with a fluorescent secondary antibody after washing, incubated at 4° C. in the dark, and detected by a flow cytometry after washing. The results are shown in Tables 3-4. After being conjugated to different ADCs, the antibodies Ab-AHZ, Ab-BHZ, and Ab-BHZm of the present disclosure could still bind to tumor cells with a high affinity. FIG. 1A, FIG. 1B and FIG. 1C show the results of the detection of binding of anti-human CDH3 antibodies and conjugates to OVCAR3 cells, HCC1569 cells and NCI-H146 cells, respectively. As shown in FIGS. 1A-1C, the antibodies and the conjugates thereof have greater binding signal values than the control PCA062 mAb.

TABLE 3
Results of the determination of cellular affinity
of anti-human CDH3 antibodies and conjugates

| | EC₅₀ (nM) |
|---|---|
| Drugs | NCI-H358 |
| Ab-BHZ | 0.51 |
| Ab-BHZ-A-05 | 0.33 |
| Ab-AHZ | 0.12 |
| Ab-AHZ-A-05 | 0.07 |

TABLE 4
Results of the determination of cellular affinity of anti-human CDH3 antibodies and conjugates

| | EC₅₀ (nM) | | | | | | | | |
|---|---|---|---|---|---|---|---|---|---|
| Drugs | HCC70 | HCC1954 | FADU | HCC1806 | OVCAR3 | SK-CO-1 | MKN45 | HCC1569 | NCI-H146 |
| Ab-BHZm | 0.10 | 0.34 | 0.16 | 0.47 | 0.20 | 0.24 | 0.13 | 0.33 | 0.36 |
| Ab-BHZm- | 0.29 | 0.29 | 0.62 | 0.43 | Not | Not | Not | Not | Not |
| A-05 | | | | | performed | performed | performed | performed | performed |
| Ab-BHZm- | 0.16 | 0.42 | 0.12 | 0.34 | Not | Not | Not | Not | Not |
| B-01 | | | | | performed | performed | performed | performed | performed |
| Ab-BHZ- | 0.25 | 0.36 | 0.36 | 0.36 | Not | Not | Not | Not | Not |
| A-14 | | | | | performed | performed | performed | performed | performed |
| Ab-BHZm- | 0.26 | 0.64 | 0.45 | 0.56 | 0.20 | 0.25 | 0.17 | 0.30 | 0.31 |
| A-14 | | | | | | | | | |

### 4.2 Detection of Protein Binding of Anti-Human CDH3 Antibodies and Conjugates Thereof

Bio-layer Interferometry (BLI) experiments were used to detect the binding of anti-human CDH3 antibodies and conjugates thereof to human CDH3 (MCE, HY-P77894), monkey CDH3 (MCE, HY-P700974), mouse CDH3 (MCE, HY-P700690), and rat CDH3 protein (Uniprot: F1LMI3, Kelun-biotech). Antibodies and conjugates thereof were diluted with a buffer DPBS (B22KJ, basalmedia)+0.025%0 Tween 20 to 5 ag/ml, and samples were loaded to a Protein A sensor (Pall); antigen proteins were diluted with the buffer to 200 nM, and then diluted in a 2-fold gradient to 1.56 nM; and association and dissociation were performed, the measured results were opened in Data Analysis 11.0 software and analyzed under a 1:1 mode by global fitting, to obtain an association rate, a dissociation rate and an affinity constant. The results are shown in Table 5. The antibodies and the conjugates thereof bound to the human and monkey CDH3 proteins, and the antibodies weakly bound to the rat CDH3 protein, and did not bind to the mouse CDH3 protein.

TABLE 5
Protein binding of anti-human CDH3 antibodies and conjugates

| | KD(M) | | | |
|---|---|---|---|---|
| | Human | Monkey | Mouse | Rat |
| Antibody | CDH3 | CDH3 | CDH3 | CDH3 |
| Ab-BHZm | 2.11E−09 | 2.58E−09 | Not binding | Weak binding |
| Ab-BHZm-A-14 | 3.46E−09 | 9.47E−10 | Not binding | Not binding |

### 4.3 Specific Detection of Anti-Human CDH3 Antibodies and Conjugates Thereof

The flow cytometry was used to detect the binding and endocytosis of antibodies and conjugates to CHO blank cells and CHO-hCDH3 cells. Cells were counted and added to a 96-well pointed bottom plate according to 2×10⁵ cells/well, and an antibody was diluted in a gradient and added with the cells, followed by the incubation at 4° C. for 1 h; after washing with a buffer, a fluorescent secondary antibody (Jackson) was added for the incubation at 4° C. for 30 min in the dark; and after washing, the flow cytometry was performed. The results are shown in FIG. 2A. The antibodies and conjugates bound to CHO-hCDH3 cells, but did not bind to CHO blank cells. The cells were cultured overnight with DMEM+20% FBS and added to a gradient-diluted antibody\pHrodo red (Thermo) the next day for the incubation at 37° C. in the dark for 24 h, and after the cells were digested and washed, the flow cytometry was performed. The results are shown in FIG. 2B. An antibody Ab-BHZm can be efficiently endocytosed to CHO-hCDH3 cells, with an endocytosis activity stronger than that of control PCA062 mAb, and all antibodies are not endocytosed to blank CHO cells. The above results showed that the antibodies and conjugates specifically bind to human CDH3 and are endocytosed to the cells expressing human CDH3, but do not bind or are not endocytosed to the CHO blank cells.

### 4.4 Detection of Endocytosis of Anti-Human CDH3 Antibodies and Conjugates Thereof

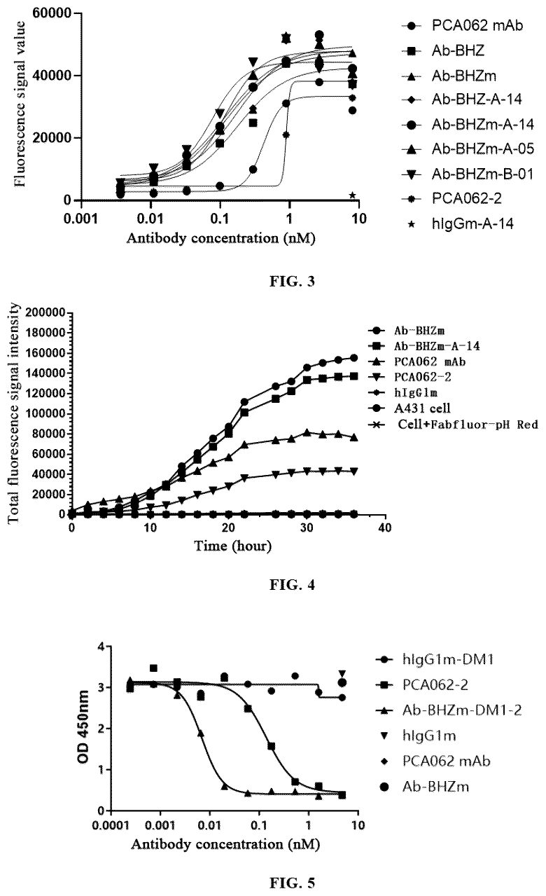

Tumor cells were digested with trypsin, counted and subsequently diluted with a complete culture medium, and added to a 96-well cell culture plate at 100 μl/well to be continuously cultured overnight. Antibodies and conjugates were diluted, incubated with a pH-sensitive fluorescent secondary antibody pHrodo red at room temperature in the dark for 15 min, added to cells and continuously cultured at 37° C. in the dark for 24 h. The cells were digested, washed with a buffer and then detected by the flow cytometry; and a fluorescence signal value was imported into software to calculate an endocytosis activity. The results are shown in Table 6 and FIG. 3 (results of the detection of endocytosis of NCI-H358 cells). The antibodies and the conjugates can be efficiently endocytosed to the tumor cells expressing CDH3, and both are superior to control antibody PCA062 mAb, with a smaller EC₅₀ and a higher endocytic signal.

A431 tumor cells were digested and collected, resuspended in a complete culture medium and subsequently counted, and added to a 96-well plate to be cultured overnight at 37° C. under 5% CO₂. The next day, the antibody, ADC and hIgG1m isotype control were diluted to 10 nM, mixed with 30 nM Incucyte® Human Fabfluor-pH Red Antibody Labeling Reagent (Sartorius), and incubated at 37° C. in the dark for 15 min; a mixture was added to the cells and cultured at 37° C. under 5% CO₂, and a fluorescence signal was read regularly by using an Incucyte® Live-Cell Analysis System and imported into software to fit the curve. The results are shown in FIG. 4 (results of the detection of endocytosis of A431 cells). The antibodies and conjugates of the present disclosure have significantly stronger endocytosis activity than control PCA062 mAb and a conjugate thereof.

TABLE 6
Assay results of cell endocytosis of anti-human CDH3 antibodies and conjugates

| | EC₅₀ (nM) | | | | | | |
|---|---|---|---|---|---|---|---|
| Drugs | NCI-H358 | HCC70 | HCC1954 | FADU | HCC1806 | HCT116 | HCC827 |
| Ab-BHZm | 0.18 | 0.29 | 0.40 | 0.17 | 0.18 | 0.16 | 0.12 |
| Ab-BHZm-A-05 | 0.12 | 0.45 | 0.21 | 0.21 | 0.15 | 0.31 | 0.38 |
| Ab-BHZm-B-01 | 0.08 | 0.14 | 0.16 | 0.16 | 0.17 | 0.13 | 0.12 |
| Ab-BHZ-A-14 | 0.12 | 0.44 | 0.20 | 0.16 | 0.19 | 0.16 | 0.31 |
| Ab-BHZm-A-14 | 0.11 | 0.28 | 0.16 | 0.10 | 0.14 | 0.12 | 0.21 |
| PCA062 mAb | 0.42 | 0.82 | 0.26 | 0.44 | 0.70 | 0.65 | 0.66 |
| PCA062-2 | 0.89 | 0.68 | 1.08 | 0.50 | 0.65 | 0.69 | 0.52 |

### 4.5 Detection of Binding of Anti-Human CDH3 Antibodies and Conjugates Thereof to FcR Proteins

The dynamic affinity of antibodies and conjugates to human Fc receptor proteins CD16a, CD32a, CD32b, CD64, and FcRn was detected by using ForteBio (Pall life sciences). The specific method was as follows: a streptavidin (SA) Sensor (Pall life sciences) was used to capture biotinylated proteins to be tested in a PBST solution, respectively, and the antibodies and conjugates to be tested were diluted with PBST to the starting concentration of 5000 nM and diluted by 2 folds to 7 concentration points; and association and dissociation were performed, and the measured results were opened in Data Analysis 11.0 software and analyzed under a 1:1 mode by global fitting, to obtain an association rate, a dissociation rate and an affinity constant. The results are shown in Table 7.

TABLE 7
Activity detection of binding of antibodies and conjugates to Fc receptors

| | KD(M) | | | | | |
|---|---|---|---|---|---|---|
| Antibody | CD16a | CD32a | CD32b | CD64 | FcRn | C1q |
| Ab-BHZm | N.D. | N.D. | N.D. | N.D. | 1.87E−8 | Weak binding |
| Ab-BHZm-A-14 | N.D. | N.D. | N.D. | N.D. | 2.21E−8 | N.D. |
| Ab-BHZ | 1.60E−06 | 1.00E−05 | 1.58E−06 | 7.96E−10 | 1.97E−8 | 2.23E−9 |

The results showed that the mutationally modified Ab-BHZm and a conjugate thereof did not bind to Fc receptors CD16a, CD32a, CD32b and CD64, weakly bound to a C1q protein, and did not have an ADCC (antibody-dependent cell-mediated cytotoxicity) activity, an ADCP (antibody-dependent cellular phagocytosis) activity or a greatly reduced CDC (complement dependent cytotoxicity) activity, which could reduce Fc receptor-mediated nonspecific killing and improve drug safety. At the same time, Ab-BHZm retained the FcRn protein binding activity and did not affect the half-life of the drug.

### 4.6 Detection of Cell Killing of Anti-Human CDH3 Conjugates

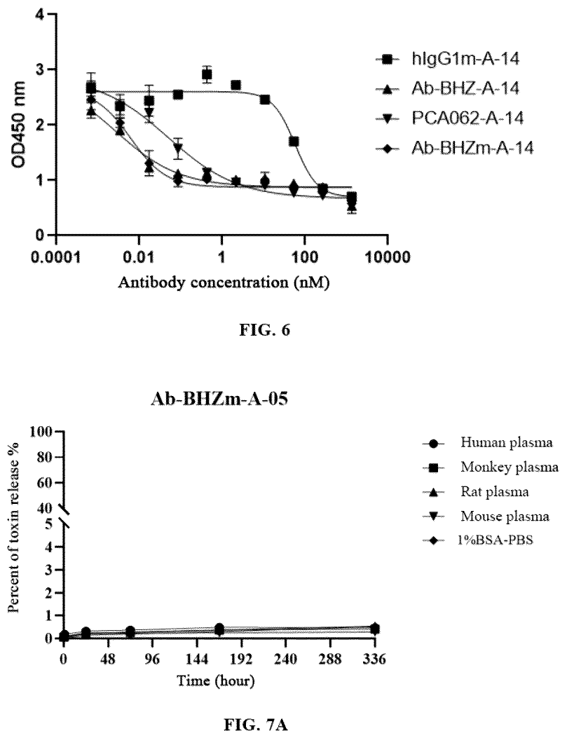

NCI-H358, HCC70, HCC1954, FADU, NCI-H146, EBC-1 (Nanjing Cobier), OVCAR3, SK-CO-1, and HT29-hCDH3 cells were digested with trypsin, and an optimal number of cells for plating were selected according to the proliferation curve of each tumor cell and cultured at 37° C. overnight. Each antibody and conjugate were diluted with a culture medium, and added to the cells to be continuously cultured for 6 d. CCK8 was added to the cells to be continuously cultured for 2-4 h. The OD450 nm numerical value was read by a microplate reader to calculate a killing activity. The results are shown in Table 8 and FIGS. 5-6. FIG. 5 shows the detection results of HCC1954 cell killing by anti-human CDH3 antibodies and the same toxin conjugates, and FIG. 6 shows detection results of HT29-hCDH3 cell killing by anti-human CDH3 antibodies and the same toxin conjugates. As shown in Table 8 and FIGS. 5-6, the killing activity of the conjugate is significantly stronger than that of a negative antibody conjugate, indicating that the killing effect is target-mediated. After conjugation with the same toxin linker, the killing activity of the antibody conjugate of the present disclosure is significantly superior to that of a control antibody conjugate.

TABLE 8
In-vitro cell killing results of anti-human CDH3 conjugates

| | IC₅₀ (nM) | | | | | | | | | |
|---|---|---|---|---|---|---|---|---|---|---|
| Drugs | NCI-H358 | HCC70 | HCC1954 | FADU | NCI-H146 | EBC-1 | MKN45 | OVCAR3 | SK-CO-1 | HT29-hCDH3 |
| hIgG1m- | 192.9 | 1980 | 3361 | Not | Not | Not | Not | Not | Not | >100 |
| A-05 | | | | detected | detected | detected | detected | detected | detected | |
| Ab-BHZm- | 25.85 | 1197 | 413.4 | Not | Not | Not | Not | Not | Not | 0.003 |
| A-05 | | | | detected | detected | detected | detected | detected | detected | |
| hIgG1m-B- | 9.37 | 8.55 | Not | Not | Not | Not | Not | Not | Not | 0.41 |
| 01 | | | detected | detected | detected | detected | detected | detected | detected | |
| Ab-BHZm- | 0.46 | 0.24 | Not | Not | Not | Not | Not | Not | Not | 0.04 |
| B-01 | | | detected | detected | detected | detected | detected | detected | detected | |
| hIgG1m- | 36.9 | 1834 | 509.4 | 55.06 | 7.95 | 66.26 | 41.80 | 14.04 | 46.47 | 25.29 |
| A-14 | | | | | | | | | | |
| Ab-BHZm- | 1.29 | 683.9 | 179.9 | 47.65 | 1.05 | 12.88 | 8.58 | 0.58 | 11.22 | 0.008 |
| A-14 | | | | | | | | | | |
| hIgG1m- | 14.73 | 14.95 | No | 51.99 | Not | Not | Not | Not | Not | Not |
| DM1 | | | killing | | detected | detected | detected | detected | detected | detected |
| Ab-BHZm- | 6.64 | <0.01 | 0.007 | 1.20 | Not | Not | Not | Not | Not | Not |
| DM1-2 | | | | | detected | detected | detected | detected | detected | detected |
| PCA062-2 | 17.68 | 0.17 | 0.14 | 5.90 | Not | Not | Not | Not | Not | Not |
| | | | | | detected | detected | detected | detected | detected | detected |

### 4.7 Plasma Stability Detection of Anti-Human CDH3 Conjugates

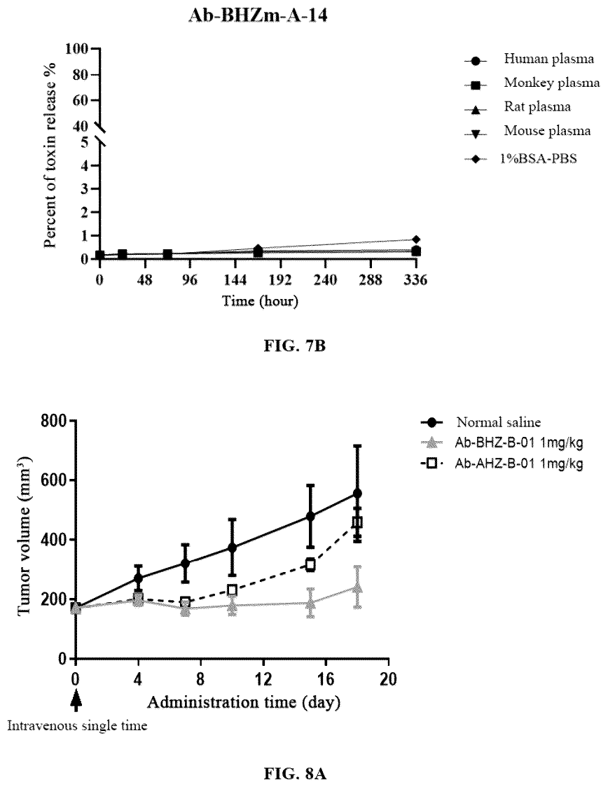

An ADC working solution was added to human, monkey, rat and mouse plasmas or 1% BSA-PBS, followed by vortical mixing; and a certain volume of plasma/1% BSA-PBS samples were taken out and added with a corresponding volume of protein precipitants 0.5% FA-acetonitrile (including internal standards) as a 0 h sample, and the remaining plasma samples were sealed with a sealing film and placed in a 37±1° C. constant temperature incubator for incubation. A certain volume of all plasma samples of the ADC-test group were taken at 1 h, 24 h, 72 h, 168 h and 336 h after incubation and added with the protein precipitant to terminate the incubation; and after protein precipitation, vortical mixing was performed at 4° C., centrifugation was performed at 13000 rpm for 10 min, and a supernatant sample was stored at −80° C. for testing. For detection, the supernatant sample was taken from −80° C. and returned to room temperature for vortical mixing; and after ultrapure water was proportionally added for dilution, vortical mixing was performed for LC-MS/MS analysis. The results are shown in FIGS. 7A-B. Ab-BHZm-A-05 and Ab-BHZm-A-14 were very stable in plasmas of different species.

### 4.8 Detection of In-Vivo Efficacy of Anti-Human CDH3 Antibody-Drug Conjugates in NCI-H358 Model

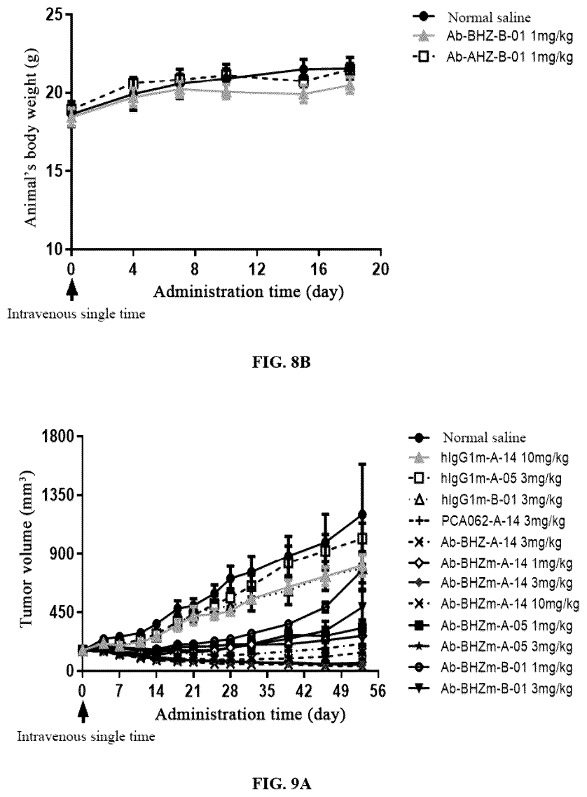

Human non-small cell lung cancer NCI-H358 cells were subjected to in-vitro monolayer culture under the culture conditions of: RPMI 1640 culture medium+10% fetal bovine serum, at 37° C. and in an incubator containing 5% CO₂ air. About 5×10⁶ NCI-H358 cells were subcutaneously inoculated in the right axilla of each mouse and suspended in 0.05 ml PBS+0.05 ml matrix gel. When an average tumor volume grew to about 150-200 mm³, the mice with too small or too large tumor volume were eliminated, and the remaining mice were randomly divided into 3 groups according to tumor volumes and animal weights, with 6 animals in each group, IV, single administration (P0). The tumor volume and the body weight were measured twice a week after administration. The specific results are shown in Table 9 and FIG. 8A-FIG. 8B. FIG. 8A shows the efficacy, and FIG. 8B shows the weight changes of the animals.

TABLE 9
Analysis of the efficacy of antibody-drug conjugates on the subcutaneous tumor-
bearing mouse model of human non-small cell lung cancer NCI-H358 cells (N = 6)

| | Administration | | | | |
|---|---|---|---|---|---|
| | dose | V_{P0} (mm³, | V_{P18} (mm³, | TGI | P |
| Drugs | (mg/kg) | mean ± SEM) | mean ± SEM) | (%) | value |
| Normal saline | / | 170.74 ± 9.14 | 555.87 ± 160.99 | / | / |
| Ab-BHZ-B-01 | 1 | 171.68 ± 6.65 | 241.66 ± 67.86 | 81.83 | 0.102 |
| Ab-AHZ-B-01 | 1 | 170.55 ± 8.36 | 459.33 ± 46.95 | 25.02 | 0.578 |

The results showed that the efficacy of Ab-BHZ-B-01 group was superior to that of Ab-AHZ-B-01 under the condition of 1 mg/kg.

### 4.9 Detection of In-Vivo Efficacy of Anti-Human CDH3 Antibody-Drug Conjugates in NCI-H358 Model

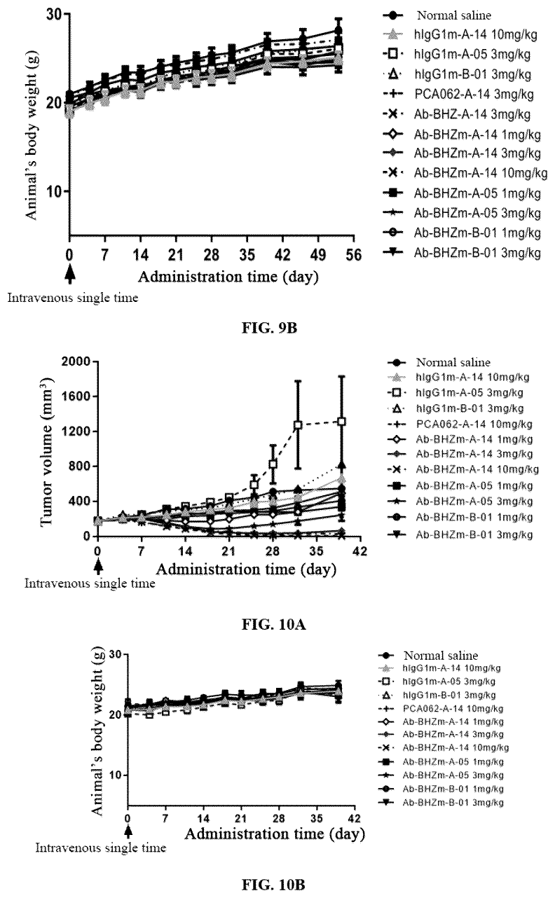

Human non-small cell lung cancer NCI-H358 cells were subjected to in-vitro monolayer culture under the culture conditions of: RPMI 1640 culture medium+10% fetal bovine serum, at 37° C. and in an incubator containing 5% CO₂ air. About 5×10⁶ NCI-H358 cells were subcutaneously inoculated in the right axilla of each mouse and suspended in 0.05 ml PBS+0.05 ml matrix gel. When an average tumor volume grew to about 150 mm³, the mice with too small or too large tumor volume were eliminated, and the remaining mice were randomly divided into 13 groups according to tumor volumes and animal weights, with 6 animals in each group, IV, single administration (P0). The tumor volume and the body weight were measured twice a week after administration. The specific results are shown in Table 10 and FIG. 9A-FIG. 9B. FIG. 9A shows the efficacy, and FIG. 9B shows the weight changes of the animals.

TABLE 10
Analysis of the efficacy of antibody-drug conjugates on the subcutaneous tumor-
bearing mouse model of human non-small cell lung cancer NCI-H358 cells (N = 6)

| | Administration | | | | |
|---|---|---|---|---|---|
| | dose | V_{P0} (mm³, | V_{P53} (mm³, | TGI | P |
| Drugs | (mg/kg) | mean ± SEM) | mean ± SEM) | (%) | value |
| Normal saline | / | 156.55 ± 6.95 | 1200.18 ± 385.51 | / | / |
| hIgG1m-A-14 | 10 | 158.74 ± 6.82 | 808.80 ± 82.98 | 37.71 | 0.344 |
| hIgG1m-A-05 | 3 | 156.88 ± 6.53 | 1016.97 ± 119.73 | 17.59 | 0.660 |
| hIgG1m-B-01 | 3 | 156.58 ± 5.93 | 785.37 ± 111.12 | 39.75 | 0.326 |
| PCA062-A-14 | 3 | 156.41 ± 7.63 | 208.86 ± 71.78 | 94.97* | 0.030 |
| Ab-BHZ-A-14 | 3 | 158.77 ± 6.66 | 143.93 ± 53.17 | 109.35* | 0.022 |
| Ab-BHZm-A-14 | 1 | 156.38 ± 5.29 | 269.66 ± 75.47 | 89.15* | 0.039 |
| Ab-BHZm-A-14 | 3 | 157.08 ± 6.53 | 45.07 ± 5.92 | 171.31* | 0.013 |
| Ab-BHZm-A-14 | 10 | 157.43 ± 5.81 | 34.71 ± 3.66 | 177.95* | 0.013 |
| Ab-BHZm-A-05 | 1 | 157.75 ± 6.01 | 328.52 ± 62.24 | 83.64* | 0.050 |
| Ab-BHZm-A-05 | 3 | 156.48 ± 6.53 | 62.11 ± 12.60 | 160.31* | 0.015 |
| Ab-BHZm-B-01 | 1 | 156.74 ± 6.05 | 771.23 ± 147.40 | 41.12 | 0.323 |
| Ab-BHZm-B-01 | 3 | 157.26 ± 5.99 | 486.36 ± 124.26 | 68.27 | 0.109 |

Note:
T-test,
*P < 0.05 indicates a significant difference compared with a normal saline group.

The results showed the conjugate of the present disclosure had a significant inhibitory effect on tumor growth in the subcutaneous tumor-bearing mouse model of human non-small cell lung cancer NCI-H358 cells. There was no animal death or animal weight loss in each administration group, and no obvious drug toxicity was observed.

### 4.10 Detection of In-Vivo Efficacy of Anti-Human CDH3 Antibody-Drug Conjugate in HCC1954 Model

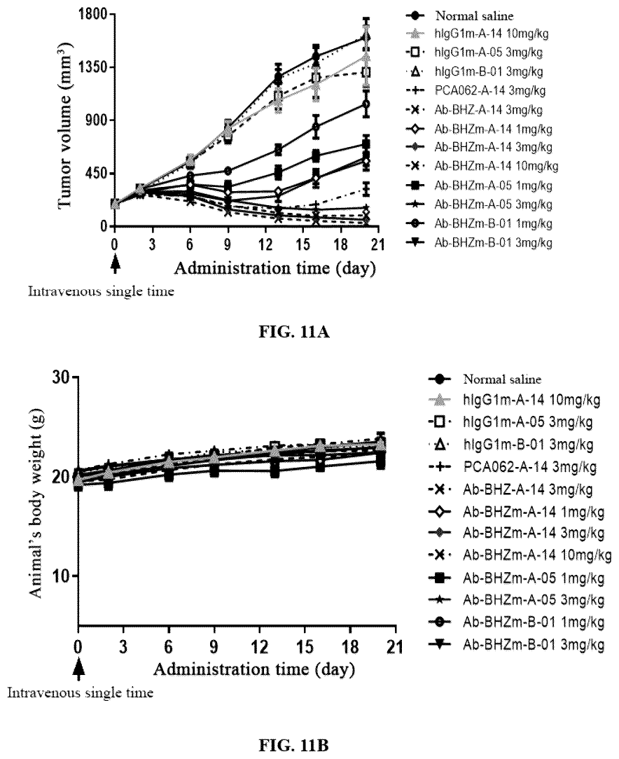

Human breast cancer HCC1954 cells were subjected to in-vitro monolayer culture under the culture conditions of: RPMI 1640 culture medium+10% fetal bovine serum, at 37° C. and in an incubator containing 5% CO₂ air. About 2.5×10⁶ HCC1954 cells were subcutaneously inoculated in the right axilla of each mouse and suspended in 0.05 ml PBS+0.05 ml matrix gel. When an average tumor volume grew to about 150-200 mm³, the mice with too small or too large tumor volume were eliminated, and the remaining mice were randomly divided into 13 groups according to tumor volumes and animal weights, with 5 animals in each group, IV, single administration (P0). The tumor volume and the body weight were measured twice a week after administration. The specific results are shown in Table 11 and FIG. 10A-FIG. 10B. FIG. 10A shows the efficacy, and FIG. 10B shows the weight changes of the animals.

TABLE 11
Analysis of the efficacy of antibody-drug conjugates on the subcutaneous
tumor-bearing mouse model of human breast cancer cells HCC1954 (N = 5)

| | Administration | | | | |
|---|---|---|---|---|---|
| | dose | V_{P0} (mm³, | V_{P39} (mm³, | TGI | P |
| Drugs | (mg/kg) | mean ± SEM) | mean ± SEM) | (%) | value |
| Normal saline | 1 | 179.08 ± 16.25 | 545.76 ± 86.46 | / | / |
| hIgG1m-A-14 | 10 | 179.27 ± 12.84 | 670.28 ± 51.61 | −33.91 | 0.251 |
| hIgG1m-A-05 | 3 | 176.56 ± 13.00 | 1313.35 ± 514.07 | −210.02 | 0.179 |
| hIgG1m-B-01 | 3 | 179.62 ± 11.05 | 824.92 ± 468.01 | −75.98 | 0.574 |
| PCA062-A-14 | 10 | 177.75 ± 18.41 | 31.24 ± 19.48 | 182.43*** | 0.000 |
| Ab-BHZm-A-14 | 1 | 180.32 ± 15.45 | 493.54 ± 120.71 | 14.58 | 0.734 |
| Ab-BHZm-A-14 | 3 | 180.54 ± 13.59 | 65.27 ± 37.53 | 163.85*** | 0.001 |
| Ab-BHZm-A-14 | 10 | 183.50 ± 20.50 | 8.29 ± 1.51 | 195.48*** | 0.000 |
| Ab-BHZm-A-05 | 1 | 175.83 ± 13.49 | 339.91 ± 40.44 | 55.25 | 0.063 |
| Ab-BHZm-A-05 | 3 | 181.12 ± 16.85 | 243.45 ± 64.52 | 83.00* | 0.023 |
| Ab-BHZm-B-01 | 1 | 181.07 ± 15.41 | 408.04 ± 76.64 | 38.10 | 0.267 |
| Ab-BHZm-B-01 | 3 | 178.57 ± 12.72 | 510.98 ± 20.56 | 9.35 | 0.706 |

Note:
T-test,
*P < 0.05,
**P < 0.01 and
***P < 0.001 indicate significant differences compared with a normal saline group.

The results showed the conjugate of the present disclosure had a significant inhibitory effect on the tumor growth of the subcutaneous tumor-bearing mouse model of human breast cancer cells HCC1954. There was no animal death or animal weight loss in each administration group, and no obvious drug toxicity was observed.

### 4.11 Detection of In-Vivo Efficacy of Anti-Human CDH3 Antibody-Drug Conjugate in HCC1806 Model

Human breast cancer HCC1806 cells were subjected to in-vitro monolayer culture under the culture conditions of: RPMI 1640 culture medium+10% fetal bovine serum, at 37° C. and in an incubator containing 5% CO₂ air. 2×10⁶ HCC1806 cells were subcutaneously inoculated in the right axilla of each mouse and suspended in 0.1 ml PBS. When an average tumor volume grew to about 150-200 mm³, the mice with too small or too large tumor volume were eliminated, and the remaining mice were randomly divided into 13 groups according to tumor volumes and animal weights, with 5 animals in each group, IV, single administration (P0). The tumor volume and the body weight were measured twice a week after administration. The specific results are shown in Table 12 and FIG. 11A-FIG. 11B. FIG. 11A shows the efficacy, and FIG. 11B shows the weight changes of the animals.

TABLE 12
Analysis of the efficacy of antibody-drug conjugates on the subcutaneous
tumor-bearing mouse model of human breast cancer cells HCC1806 (N = 6)

| | Administration | | | | |
|---|---|---|---|---|---|
| | dose | V_{P0} (mm³, | V_{P20} (mm³, | TGI | P |
| Drugs | (mg/kg) | mean ± SEM) | mean ± SEM) | (%) | value |
| Normal saline | 1 | 190.16 ± 8.67 | 1603.35 ± 95.11 | / | / |
| hIgG1m-A-14 | 10 | 192.54 ± 11.79 | 1445.88 ± 248.68 | 11.31 | 0.541 |
| hIgG1m-A-05 | 3 | 191.77 ± 11.06 | 1308.54 ± 121.46 | 20.97 | 0.085 |
| hIgG1m-B-01 | 3 | 190.54 ± 13.45 | 1629.55 ± 131.87 | −1.83 | 0.875 |
| PCA062-A-14 | 3 | 187.28 ± 11.67 | 317.47 ± 54.17 | 90.79*** | 0.000 |
| Ab-BHZ-A-14 | 3 | 192.02 ± 10.66 | 94.49 ± 31.63 | 150.79*** | 0.000 |
| Ab-BHZm-A-14 | 1 | 189.32 ± 9.34 | 556.20 ± 39.83 | 74.04*** | 0.000 |
| Ab-BHZm-A-14 | 3 | 189.83 ± 11.01 | 55.52 ± 9.00 | 170.75*** | 0.000 |
| Ab-BHZm-A-14 | 10 | 190.94 ± 12.48 | 28.86 ± 4.70 | 184.89*** | 0.000 |
| Ab-BHZm-A-05 | 1 | 187.04 ± 11.36 | 699.97 ± 71.89 | 63.70*** | 0.000 |
| Ab-BHZm-A-05 | 3 | 191.70 ± 14.13 | 158.57 ± 25.16 | 117.28*** | 0.000 |
| Ab-BHZm-B-01 | 1 | 190.51 ± 7.93 | 1038.92 ± 109.11 | 39.97** | 0.003 |
| Ab-BHZm-B-01 | 3 | 190.91 ± 9.62 | 587.69 ± 108.05 | 71.92*** | 0.000 |

Note:
T-test,
**P < 0.01 and
***P < 0.001 indicate significant differences compared with a normal saline group.

The results showed the conjugate of the present disclosure had a significant inhibitory effect on the tumor growth of the subcutaneous tumor-bearing mouse model of human breast cancer cells HCC1806. There was no animal death or animal weight loss in each administration group, and no obvious drug toxicity was observed.

### 4.12 Detection of In-Vivo Efficacy of Anti-Human CDH3 Antibody-Drug Conjugate in FADU Model

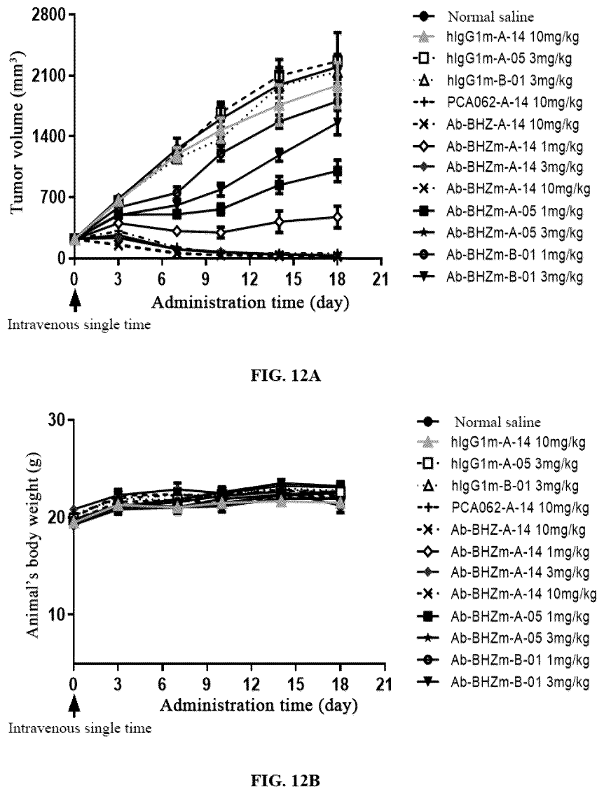

Human pharyngeal squamous cell carcinoma FADU cells were subjected to in-vitro monolayer culture under the culture conditions of an addition of 10% fetal bovine serum to an EMEM culture medium, at 37° C. and in an incubator containing 5% CO₂ air. 5×10⁶ FADU cells were subcutaneously inoculated in the right axilla of each mouse and suspended in 0.05 ml PBS+0.05 ml matrix gel. When an average tumor volume grew to about 200-250 mm³, the mice with too small or too large tumor volume were eliminated, and the remaining mice were randomly divided into 13 groups according to tumor volumes and animal weights, with 6 animals in each group, IV, single administration (P0). The tumor volume and the body weight were measured twice a week after administration. The specific results are shown in Table 13 and FIG. 12A-FIG. 12B. FIG. 12A shows the efficacy, and FIG. 12B shows the weight changes of the animals.

TABLE 13
Analysis of the efficacy of antibody-drug conjugates on the subcutaneous tumor-
bearing mouse model of human pharyngeal squamous cell carcinoma FADU (N = 6)

| | Administration | | | | |
|---|---|---|---|---|---|
| | dose | V_{P0} (mm³, | V_{P18} (mm³, | TGI | P |
| Drugs | (mg/kg) | mean ± SEM) | mean ± SEM) | (%) | value |
| Normal saline | / | 221.78 ± 8.99 | 2198.70 ± 145.41 | / | / |
| hIgG1m-A-14 | 10 | 220.75 ± 10.25 | 1988.06 ± 260.04 | 10.60 | 0.496 |
| hIgG1m-A-05 | 3 | 218.93 ± 6.95 | 2264.03 ± 330.14 | −3.45 | 0.860 |
| hIgG1m-B-01 | 3 | 221.29 ± 7.05 | 2142.06 ± 141.62 | 2.84 | 0.786 |
| PCA062-A-14 | 10 | 223.17 ± 8.04 | 57.89 ± 8.20 | 174.06*** | 0.000 |
| Ab-BHZ-A-14 | 10 | 219.01 ± 8.97 | 25.07 ± 5.55 | 188.55*** | 0.000 |
| Ab-BHZm-A-14 | 1 | 218.10 ± 11.76 | 471.96 ± 124.36 | 87.16*** | 0.000 |
| Ab-BHZm-A-14 | 3 | 220.56 ± 6.60 | 33.47 ± 4.58 | 184.83*** | 0.000 |
| Ab-BHZm-A-14 | 10 | 219.20 ± 9.84 | 16.83 ± 3.23 | 192.32*** | 0.000 |
| Ab-BHZm-A-05 | 1 | 222.34 ± 6.46 | 1005.18 ± 125.17 | 60.40*** | 0.000 |
| Ab-BHZm-A-05 | 3 | 217.94 ± 11.46 | 27.38 ± 2.30 | 187.44*** | 0.000 |
| Ab-BHZm-B-01 | 1 | 219.73 ± 6.44 | 1802.34 ± 77.28 | 19.95* | 0.037 |
| Ab-BHZm-B-01 | 3 | 220.46 ± 10.85 | 1557.80 ± 139.18 | 32.35** | 0.010 |

Note:
T-test,
**P < 0.01 and
***P < 0.001 indicate significant differences compared with a normal saline group.

The results showed the conjugates of the present disclosure have a significant inhibitory effect on the tumor growth of the FADU model. There was no animal death or animal weight loss in each administration group, and no obvious drug toxicity was observed.

1. An antibody-drug conjugate comprising a structure shown in formula (I): 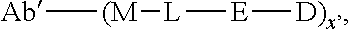
wherein:
Ab′ is an antibody or an antigen-binding fragment thereof, which specifically binds to CDH3;
M is a linker connected to the antibody or the antigen-binding fragment thereof,
L is a moiety connecting the linker M and E;
E is a moiety connecting L and D;
D is a cytotoxic drug moiety; and
x′ is an integer selected from 1, 2, 3, 4, 5, 6, 7, 8, 9, 10, 11, 12, 13, 14 15, 16, 17, 18, 19, and 20.

2. The antibody-drug conjugate of claim 1, wherein the antibody or the antigen-binding fragment thereof comprises complementarity determining regions (CDRs) as follows:
(a) CDR-H1, CDR-H2 and CDR-H3 amino acid sequences of a heavy chain variable region (VH) amino acid sequence set forth in SEQ ID NO: 3 or 1; and/or CDR-L1, CDR-L2 and CDR-L3 amino acid sequences of a light chain variable region (VL) amino acid sequence set forth in SEQ ID NO: 4 or 2;
(b) CDR-H1, CDR-H2 and CDR-H3 amino acid sequences of a VH amino acid sequence set forth in SEQ ID NO: 7 or 5; and/or CDR-L1, CDR-L2 and CDR-L3 amino acid sequences of a VL amino acid sequence set forth in SEQ ID NO: 8 or 6;
(c) CDR-H1, CDR-H2 and CDR-H3 amino acid sequences of a VH amino acid sequence set forth in SEQ ID NO: 11 or 9; and/or CDR-L1, CDR-L2 and CDR-L3 amino acid sequences of a VL amino acid sequence set forth in SEQ ID NO: 12 or 10;
(d) CDR-H1, CDR-H2 and CDR-H3 amino acid sequences of a VH amino acid sequence set forth in SEQ ID NO: 15 or 13; and/or CDR-L1, CDR-L2 and CDR-L3 amino acid sequences of a VL amino acid sequence set forth in SEQ ID NO: 14 or 12; or
(e) CDR-H1, CDR-H2 and CDR-H3 amino acid sequences of a VH, and/or CDR-L1, CDR-L2 and CDR-L3 amino acid sequences of a VL, wherein at least one CDR of the VH and/or VL comprises a mutation compared to the VH and/or the VL of any one of (a) to (d), and the mutation is a substitution, deletion or addition of one, two or several amino acids (for example, a substitution, deletion or addition of 1, 2, or 3 amino acids);
wherein the CDRs are defined according to the Chothia, AbM, Kabat, Contact, or IMGT numbering system.

3. (canceled)

4. The antibody-drug conjugate of claim 1, wherein the antibody or the antigen-binding fragment thereof comprises:
(a) CDR-H1, CDR-H2 and CDR-H3 amino acid sequences of the VH amino acid sequence set forth in SEQ ID NO: 3; and CDR-L1, CDR-L2 and CDR-L3 amino acid sequences of the VL amino acid sequence set forth in SEQ ID NO: 4;
(b) CDR-H1, CDR-H2 and CDR-H3 amino acid sequences of the VH amino acid sequence set forth in SEQ ID NO: 1; and CDR-L1, CDR-L2 and CDR-L3 amino acid sequences of the VL amino acid sequence set forth in SEQ ID NO: 2;
(c) CDR-H1, CDR-H2 and CDR-H3 amino acid sequences of the VH amino acid sequence set forth in SEQ ID NO: 7; and CDR-L1, CDR-L2 and CDR-L3 amino acid sequences of the VL amino acid sequence set forth in SEQ ID NO: 8;
(d) CDR-H1, CDR-H2 and CDR-H3 amino acid sequences of the VH amino acid sequence set forth in SEQ ID NO: 5; and CDR-L1, CDR-L2 and CDR-L3 amino acid sequences of the VL amino acid sequence set forth in SEQ ID NO: 6;
(e) CDR-H1, CDR-H2 and CDR-H3 amino acid sequences of the VH amino acid sequence set forth in SEQ ID NO: 11; and CDR-L1, CDR-L2 and CDR-L3 amino acid sequences of the VL amino acid sequence set forth in SEQ ID NO: 12;
(f) CDR-H1, CDR-H2 and CDR-H3 amino acid sequences of the VH amino acid sequence set forth in SEQ ID NO: 9; and CDR-L1, CDR-L2 and CDR-L3 amino acid sequences of the VL amino acid sequence set forth in SEQ ID NO: 10;
(g) CDR-H1, CDR-H2 and CDR-H3 amino acid sequences of the VH amino acid sequence set forth in SEQ ID NO: 15; and CDR-L1, CDR-L2 and CDR-L3 amino acid sequences of the VL amino acid sequence set forth in SEQ ID NO: 16; or
(h) CDR-H1, CDR-H2 and CDR-H3 amino acid sequences of the VH amino acid sequence set forth in SEQ ID NO: 13; and CDR-L1, CDR-L2 and CDR-L3 amino acid sequences of the VL amino acid sequence set forth in SEQ ID NO: 14.

5.-6. (canceled)

7. The antibody-drug conjugate of claim 1, wherein the antibody or the antigen-binding fragment thereof comprises:
(1) the following VH and/or VL, wherein the CDRs are defined according to the Chothia numbering system:
(1a) a VH comprising CDR-H1 comprising the amino acid sequence of SEQ ID NO: 17; CDR-H2 comprising the amino acid sequence of SEQ ID NO: 27; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42;
(1b) a VH comprising CDR-H1 comprising the amino acid sequence of SEQ ID NO: 17; CDR-H2 comprising the amino acid sequence of SEQ ID NO: 22; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42;
(1c) a VH comprising CDR-H1 comprising the amino acid sequence of SEQ ID NO: 44; CDR-H2 comprising the amino acid sequence of SEQ ID NO: 49; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 63; and CDR-L3 comprising the amino acid sequence of SEQ ID NO: 66;
(1d) a VH comprising CDR-H1 comprising the amino acid sequence of SEQ ID NO: 44; CDR-H2 comprising the amino acid sequence of SEQ ID NO: 49; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 61; and CDR-L3 comprising the amino acid sequence of SEQ ID NO: 66;
(1e) a VH comprising CDR-H1 comprising the amino acid sequence of SEQ ID NO: 68; CDR-H2 comprising the amino acid sequence of SEQ ID NO: 73; and CDR-H3 comprising the amino acid sequence of SEQ ID NO: 80; and/or a VL comprising CDR-L1 comprising the amino acid sequence of SEQ ID NO: 84; CDR-L2 comprising the amino acid sequence of SEQ ID NO: 88; and CDR-L3 comprising the amino acid sequence of SEQ ID NO: 91;
(1f) a VH comprising CDR-H1 comprising the amino acid sequence of SEQ ID NO: 68; CDR-H2 comprising the amino acid sequence of SEQ ID NO: 73; and CDR-H3 comprising the amino acid sequence of SEQ ID NO: 80; and/or a VL comprising CDR-L1 comprising the amino acid sequence of SEQ ID NO: 83; CDR-L2 comprising the amino acid sequence of SEQ ID NO: 87; and CDR-L3 comprising the amino acid sequence of SEQ ID NO: 91;
(1g) a VH comprising CDR-H1 comprising the amino acid sequence of SEQ ID NO: 93; CDR-H2 comprising the amino acid sequence of SEQ ID NO: 103; and CDR-H3 comprising the amino acid sequence of SEQ ID NO: 108; and/or a VL comprising CDR-L1 comprising the amino acid sequence of SEQ ID NO: 114; CDR-L2 comprising the amino acid sequence of SEQ ID NO: 117; and CDR-L3 comprising the amino acid sequence of SEQ ID NO: 119; or
(1h) a VH comprising CDR-H1 comprising the amino acid sequence of SEQ ID NO: 93; CDR-H2 comprising the amino acid sequence of SEQ ID NO: 98; and CDR-H3 comprising the amino acid sequence of SEQ ID NO: 108; and/or a VL comprising CDR-L1 comprising the amino acid sequence of SEQ ID NO: 111; CDR-L2 comprising the amino acid sequence of SEQ ID NO: 117; and CDR-L3 comprising the amino acid sequence of SEQ ID NO: 119;
or
(2) the following VH and/or VL, wherein the CDRs are defined according to the AbM numbering system:
(2a) a VH comprising CDR-H1 comprising the amino acid sequence of SEQ ID NO: 18; CDR-H2 comprising the amino acid sequence of SEQ ID NO: 28; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42;
(2b) a VH comprising CDR-H1 comprising the amino acid sequence of SEQ ID NO: 18; CDR-H2 comprising the amino acid sequence of SEQ ID NO: 23; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42;
(2c) a VH comprising CDR-H1 comprising the amino acid sequence of SEQ ID NO: 45; CDR-H2 comprising the amino acid sequence of SEQ ID NO: 50; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 63; and CDR-L3 comprising the amino acid sequence of SEQ ID NO: 66;
(2d) a VH comprising CDR-H1 comprising the amino acid sequence of SEQ ID NO: 45; CDR-H2 comprising the amino acid sequence of SEQ ID NO: 50; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 61; and CDR-L3 comprising the amino acid sequence of SEQ ID NO: 66;
(2e) a VH comprising CDR-H1 comprising the amino acid sequence of SEQ ID NO: 69; CDR-H2 comprising the amino acid sequence of SEQ ID NO: 74; and CDR-H3 comprising the amino acid sequence of SEQ ID NO: 80; and/or a VL comprising CDR-L1 comprising the amino acid sequence of SEQ ID NO: 84; CDR-L2 comprising the amino acid sequence of SEQ ID NO: 88; and CDR-L3 comprising the amino acid sequence of SEQ ID NO: 91;
(2f) a VH comprising CDR-H1 comprising the amino acid sequence of SEQ ID NO: 69; CDR-H2 comprising the amino acid sequence of SEQ ID NO: 74; and CDR-H3 comprising the amino acid sequence of SEQ ID NO: 80; and/or a VL comprising CDR-L1 comprising the amino acid sequence of SEQ ID NO: 83; CDR-L2 comprising the amino acid sequence of SEQ ID NO: 87; and CDR-L3 comprising the amino acid sequence of SEQ ID NO: 91;
(2g) a VH comprising CDR-H1 comprising the amino acid sequence of SEQ ID NO: 94; CDR-H2 comprising the amino acid sequence of SEQ ID NO: 104; and CDR-H3 comprising the amino acid sequence of SEQ ID NO: 108; and/or a VL comprising CDR-L1 comprising the amino acid sequence of SEQ ID NO: 114; CDR-L2 comprising the amino acid sequence of SEQ ID NO: 117; and CDR-L3 comprising the amino acid sequence of SEQ ID NO: 119; or
(2h) a VH comprising CDR-H1 comprising the amino acid sequence of SEQ ID NO: 94; CDR-H2 comprising the amino acid sequence of SEQ ID NO: 99; and CDR-H3 comprising the amino acid sequence of SEQ ID NO: 108; and/or a VL comprising CDR-L1 comprising the amino acid sequence of SEQ ID NO: 111; CDR-L2 comprising the amino acid sequence of SEQ ID NO: 117; and CDR-L3 comprising the amino acid sequence of SEQ ID NO: 119;
or
(3) the following VH and/or VL, wherein the CDRs are defined according to the Kabat numbering system:
(3a) a VH comprising CDR-H1 comprising the amino acid sequence of SEQ ID NO: 19; CDR-H2 comprising the amino acid sequence of SEQ ID NO: 29; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42;
(3b) a VH comprising CDR-H1 comprising the amino acid sequence of SEQ ID NO: 19; CDR-H2 comprising the amino acid sequence of SEQ ID NO: 24; and CDR-H3 comprising the amino acid sequence of SEQ ID NO: 32; and/or a VL comprising CDR-L1 comprising the amino acid sequence of SEQ ID NO: 35; CDR-L2 comprising the amino acid sequence of SEQ ID NO: 38; and CDR-L3 comprising the amino acid sequence of SEQ ID NO: 42;
(3c) a VH comprising CDR-H1 comprising the amino acid sequence of SEQ ID NO: 46; CDR-H2 comprising the amino acid sequence of SEQ ID NO: 52; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 63; and CDR-L3 comprising the amino acid sequence of SEQ ID NO: 66;
(3d) a VH comprising CDR-H1 comprising the amino acid sequence of SEQ ID NO: 46; CDR-H2 comprising the amino acid sequence of SEQ ID NO: 51; and CDR-H3 comprising the amino acid sequence of SEQ ID NO: 55; and/or a VL comprising CDR-L1 comprising the amino acid sequence of SEQ ID NO: 58; CDR-L2 comprising the amino acid sequence of SEQ ID NO: 61; and CDR-L3 comprising the amino acid sequence of SEQ ID NO: 66;
(3e) a VH comprising CDR-H1 comprising the amino acid sequence of SEQ ID NO: 70; CDR-H2 comprising the amino acid sequence of SEQ ID NO: 77; and CDR-H3 comprising the amino acid sequence of SEQ ID NO: 80; and/or a VL comprising CDR-L1 comprising the amino acid sequence of SEQ ID NO: 84; CDR-L2 comprising the amino acid sequence of SEQ ID NO: 88; and CDR-L3 comprising the amino acid sequence of SEQ ID NO: 91;
(3f) a VH comprising CDR-H1 comprising the amino acid sequence of SEQ ID NO: 70; CDR-H2 comprising the amino acid sequence of SEQ ID NO: 75; and CDR-H3 comprising the amino acid sequence of SEQ ID NO: 80; and/or a VL comprising CDR-L1 comprising the amino acid sequence of SEQ ID NO: 83; CDR-L2 comprising the amino acid sequence of SEQ ID NO: 87; and CDR-L3 comprising the amino acid sequence of SEQ ID NO: 91;
(3g) a VH comprising CDR-H1 comprising the amino acid sequence of SEQ ID NO: 95; CDR-H2 comprising the amino acid sequence of SEQ ID NO: 105; and CDR-H3 comprising the amino acid sequence of SEQ ID NO: 108; and/or a VL comprising CDR-L1 comprising the amino acid sequence of SEQ ID NO: 114; CDR-L2 comprising the amino acid sequence of SEQ ID NO: 117; and CDR-L3 comprising the amino acid sequence of SEQ ID NO: 119; or
(3h) a VH comprising CDR-H1 comprising the amino acid sequence of SEQ ID NO: 95; CDR-H2 comprising the amino acid sequence of SEQ ID NO: 100; and CDR-H3 comprising the amino acid sequence of SEQ ID NO: 108; and/or a VL comprising CDR-L1 comprising the amino acid sequence of SEQ ID NO: 111; CDR-L2 comprising the amino acid sequence of SEQ ID NO: 117; and CDR-L3 comprising the amino acid sequence of SEQ ID NO: 119;
or
(4) the following VH and/or VL, wherein the CDRs are defined according to the Contact numbering system:
(4a) a VH comprising CDR-H1 comprising the amino acid sequence of SEQ ID NO: 20; CDR-H2 comprising the amino acid sequence of SEQ ID NO: 30; and CDR-H3 comprising the amino acid sequence of SEQ ID NO: 33; and/or a VL comprising CDR-L1 comprising the amino acid sequence of SEQ ID NO: 36; CDR-L2 comprising the amino acid sequence of SEQ ID NO: 40; and CDR-L3 comprising the amino acid sequence of SEQ ID NO: 43;
(4b) a VH comprising CDR-H1 comprising the amino acid sequence of SEQ ID NO: 20; CDR-H2 comprising the amino acid sequence of SEQ ID NO: 25; and CDR-H3 comprising the amino acid sequence of SEQ ID NO: 33; and/or a VL comprising CDR-L1 comprising the amino acid sequence of SEQ ID NO: 36; CDR-L2 comprising the amino acid sequence of SEQ ID NO: 39; and CDR-L3 comprising the amino acid sequence of SEQ ID NO: 43;
(4c) a VH comprising CDR-H1 comprising the amino acid sequence of SEQ ID NO: 47; CDR-H2 comprising the amino acid sequence of SEQ ID NO: 53; and CDR-H3 comprising the amino acid sequence of SEQ ID NO: 56; and/or a VL comprising CDR-L1 comprising the amino acid sequence of SEQ ID NO: 59; CDR-L2 comprising the amino acid sequence of SEQ ID NO: 64; and CDR-L3 comprising the amino acid sequence of SEQ ID NO: 67;
(4d) a VH comprising CDR-H1 comprising the amino acid sequence of SEQ ID NO: 47; CDR-H2 comprising the amino acid sequence of SEQ ID NO: 53; and CDR-H3 comprising the amino acid sequence of SEQ ID NO: 56; and/or a VL comprising CDR-L1 comprising the amino acid sequence of SEQ ID NO: 59; CDR-L2 comprising the amino acid sequence of SEQ ID NO: 62; and CDR-L3 comprising the amino acid sequence of SEQ ID NO: 67;
(4e) a VH comprising CDR-H1 comprising the amino acid sequence of SEQ ID NO: 71; CDR-H2 comprising the amino acid sequence of SEQ ID NO: 78; and CDR-H3 comprising the amino acid sequence of SEQ ID NO: 81; and/or a VL comprising CDR-L1 comprising the amino acid sequence of SEQ ID NO: 85; CDR-L2 comprising the amino acid sequence of SEQ ID NO: 89; and CDR-L3 comprising the amino acid sequence of SEQ ID NO: 92;
(4f) a VH comprising CDR-H1 comprising the amino acid sequence of SEQ ID NO: 71; CDR-H2 comprising the amino acid sequence of SEQ ID NO: 76; and CDR-H3 comprising the amino acid sequence of SEQ ID NO: 81; and/or a VL comprising CDR-L1 comprising the amino acid sequence of SEQ ID NO: 85; CDR-L2 comprising the amino acid sequence of SEQ ID NO: 89; and CDR-L3 comprising the amino acid sequence of SEQ ID NO: 92;
(4g) a VH comprising CDR-H1 comprising the amino acid sequence of SEQ ID NO: 96; CDR-H2 comprising the amino acid sequence of SEQ ID NO: 106; and CDR-H3 comprising the amino acid sequence of SEQ ID NO: 109; and/or a VL comprising CDR-L1 comprising the amino acid sequence of SEQ ID NO: 115; CDR-L2 comprising the amino acid sequence of SEQ ID NO: 118; and CDR-L3 comprising the amino acid sequence of SEQ ID NO: 120; or
(4h) a VH comprising CDR-H1 comprising the amino acid sequence of SEQ ID NO: 96; CDR-H2 comprising the amino acid sequence of SEQ ID NO: 101; and CDR-H3 comprising the amino acid sequence of SEQ ID NO: 109; and/or a VL comprising CDR-L1 comprising the amino acid sequence of SEQ ID NO: 112; CDR-L2 comprising the amino acid sequence of SEQ ID NO: 118; and CDR-L3 comprising the amino acid sequence of SEQ ID NO: 120;
or
(5) the following VH and/or VL, wherein the CDRs are defined according to the IMGT numbering system:
(5a) a VH comprising CDR-H1 comprising the amino acid sequence of SEQ ID NO: 21; CDR-H2 comprising the amino acid sequence of SEQ ID NO: 31; and CDR-H3 comprising the amino acid sequence of SEQ ID NO: 34; and/or a VL comprising CDR-L1 comprising the amino acid sequence of SEQ ID NO: 37; CDR-L2 comprising the amino acid sequence of SEQ ID NO: 41; and CDR-L3 comprising the amino acid sequence of SEQ ID NO: 42;
(5b) a VH comprising CDR-H1 comprising the amino acid sequence of SEQ ID NO: 21; CDR-H2 comprising the amino acid sequence of SEQ ID NO: 26; and CDR-H3 comprising the amino acid sequence of SEQ ID NO: 34; and/or a VL comprising CDR-L1 comprising the amino acid sequence of SEQ ID NO: 37; CDR-L2 comprising the amino acid sequence of SEQ ID NO: 41; and CDR-L3 comprising the amino acid sequence of SEQ ID NO: 42;
(5c) a VH comprising CDR-H1 comprising the amino acid sequence of SEQ ID NO: 48; CDR-H2 comprising the amino acid sequence of SEQ ID NO: 54; and CDR-H3 comprising the amino acid sequence of SEQ ID NO: 57; and/or a VL comprising CDR-L1 comprising the amino acid sequence of SEQ ID NO: 60; CDR-L2 comprising the amino acid sequence of SEQ ID NO: 65; and CDR-L3 comprising the amino acid sequence of SEQ ID NO: 66;
(5d) a VH comprising CDR-H1 comprising the amino acid sequence of SEQ ID NO: 72; CDR-H2 comprising the amino acid sequence of SEQ ID NO: 79; and CDR-H3 comprising the amino acid sequence of SEQ ID NO: 82; and/or a VL comprising CDR-L1 comprising the amino acid sequence of SEQ ID NO: 86; CDR-L2 comprising the amino acid sequence of SEQ ID NO: 90; and CDR-L3 comprising the amino acid sequence of SEQ ID NO: 91;
(5e) a VH comprising CDR-H1 comprising the amino acid sequence of SEQ ID NO: 97; CDR-H2 comprising the amino acid sequence of SEQ ID NO: 107; and CDR-H3 comprising the amino acid sequence of SEQ ID NO: 110; and/or a VL comprising CDR-L1 comprising the amino acid sequence of SEQ ID NO: 116; CDR-L2 comprising the amino acid sequence of SEQ ID NO: 41; and CDR-L3 comprising the amino acid sequence of SEQ ID NO: 119; or
(5f) a VH comprising CDR-H1 comprising the amino acid sequence of SEQ ID NO: 97; CDR-H2 comprising the amino acid sequence of SEQ ID NO: 102; and CDR-H3 comprising the amino acid sequence of SEQ ID NO: 110; and/or a VL comprising CDR-L1 comprising the amino acid sequence of SEQ ID NO: 113; CDR-L2 comprising the amino acid sequence of SEQ ID NO: 41; and CDR-L3 comprising the amino acid sequence of SEQ ID NO: 119.

8. The antibody-drug conjugate of claim 1, wherein the antibody or the antigen-binding fragment thereof comprises:
(a) a VH comprising the amino acid sequence set forth in SEQ ID NO: 3 or a variant thereof and/or a VL comprising the amino acid sequence set forth in SEQ ID NO: 4 or a variant thereof,
(b) a VH comprising the amino acid sequence set forth in SEQ ID NO: 1 or a variant thereof and/or a VL comprising the amino acid sequence set forth in SEQ ID NO: 2 or a variant thereof,
(c) a VH comprising the amino acid sequence set forth in SEQ ID NO: 7 or a variant thereof and/or a VL comprising the amino acid sequence set forth in SEQ ID NO: 8 or a variant thereof,
(d) a VH comprising the amino acid sequence set forth in SEQ ID NO: 5 or a variant thereof and/or a VL comprising the amino acid sequence set forth in SEQ ID NO: 6 or a variant thereof,
(e) a VH comprising the amino acid sequence set forth in SEQ ID NO: 11 or a variant thereof and/or a VL comprising the amino acid sequence set forth in SEQ ID NO: 12 or a variant thereof,
(f) a VH comprising the amino acid sequence set forth in SEQ ID NO: 9 or a variant thereof and/or a VL comprising the amino acid sequence set forth in SEQ ID NO: 10 or a variant thereof,
(g) a VH comprising the amino acid sequence set forth in SEQ ID NO: 15 or a variant thereof and/or a VL comprising the amino acid sequence set forth in SEQ ID NO: 16 or a variant thereof, or
(h) a VH comprising the amino acid sequence set forth in SEQ ID NO: 13 or a variant thereof and/or a VL comprising the amino acid sequence set forth in SEQ ID NO: 14 or a variant thereof,
wherein the variant has at least 80%, at least 85%, at least 90%, at least 91%, at least 92%, at least 93%, at least 94%, at least 95%, at least 96%, at least 97%, at least 98%, at least 99% or 100% sequence identity compared to the sequence from which it is derived, or has a substitution, deletion or addition of one, two or several amino acids (for example, a substitution, deletion or addition of 1, 2, 3, 4, or 5 amino acids) compared to the sequence from which it is derived.

9. (canceled)

10. The antibody-drug conjugate of claim 1, wherein the antibody or the antigen-binding fragment thereof comprises:
(a) a VH comprising the amino acid sequence set forth in SEQ ID NO: 3 and a VL comprising the amino acid sequence set forth in SEQ ID NO: 4;
(b) a VH comprising the amino acid sequence set forth in SEQ ID NO: 1 and a VL comprising the amino acid sequence set forth in SEQ ID NO: 2;
(c) a VH comprising the amino acid sequence set forth in SEQ ID NO: 7 and a VL comprising the amino acid sequence set forth in SEQ ID NO: 8;
(d) a VH comprising the amino acid sequence set forth in SEQ ID NO: 5 and a VL comprising the amino acid sequence set forth in SEQ ID NO: 6;
(e) a VH comprising the amino acid sequence set forth in SEQ ID NO: 11 and a VL comprising the amino acid sequence set forth in SEQ ID NO: 12;
(f) a VH comprising the amino acid sequence set forth in SEQ ID NO: 9 and a VL comprising the amino acid sequence set forth in SEQ ID NO: 10;
(g) a VH comprising the amino acid sequence set forth in SEQ ID NO: 15 and a VL comprising the amino acid sequence set forth in SEQ ID NO: 16; or
(h) a VH comprising the amino acid sequence set forth in SEQ ID NO: 13 and a VL comprising the amino acid sequence set forth in SEQ ID NO: 14.

11. The antibody-drug conjugate of claim 1, wherein the antibody or the antigen-binding fragment thereof is a murine antibody, a chimeric antibody, or a humanized antibody.

12.-16. (canceled)

17. The antibody-drug conjugate of claim 1, wherein the antibody or the antigen-binding fragment thereof comprises a heavy chain constant region (CH) comprising the amino acid sequence set forth in SEQ ID NO: 121 or a variant thereof, and the variant has a conservative substitution of at most 20 amino acids compared to SEQ ID NO: 121.

18. The antibody-drug conjugate of claim 1, wherein the antibody or the antigen-binding fragment thereof comprises a light chain constant region (CL) comprising the amino acid sequence set forth in SEQ ID NO: 123 or 144 or a variant thereof, and the variant has a conservative substitution of at most 20 amino acids compared to SEQ ID NO: 123 or 144.

19. The antibody-drug conjugate of claim 1, wherein the antibody or the antigen-binding fragment thereof comprises a variant of the human IgG1 heavy chain constant region comprising the amino acid sequence set forth in SEQ ID NO: 122.

20. (canceled)

21. The antibody-drug conjugate of claim 17, wherein the CH comprising the amino acid sequence set forth in SEQ ID NO: 121 or 122 or the variant thereof lacks C-terminal lysine.

22. The antibody-drug conjugate of claim 1, wherein the antibody or the antigen-binding fragment thereof comprises:
(1) a heavy chain comprising a VH comprising the amino acid sequence set forth in SEQ ID NO: 3 and a CH comprising the amino acid sequence set forth in SEQ ID NO: 122, and a light chain comprising a VL comprising the amino acid sequence set forth in SEQ ID NO: 4 and a CL comprising the amino acid sequence set forth in SEQ ID NO: 123;
(2) a heavy chain comprising a VH comprising the amino acid sequence set forth in SEQ ID NO: 3 and a CH comprising the amino acid sequence set forth in SEQ ID NO: 121, and a light chain comprising a VL comprising the amino acid sequence set forth in SEQ ID NO: 4 and a CL comprising the amino acid sequence set forth in SEQ ID NO: 123;
(3) a heavy chain comprising a VH comprising the amino acid sequence set forth in SEQ ID NO: 1 and a CH comprising the amino acid sequence set forth in SEQ ID NO: 121, and a light chain comprising a VL comprising the amino acid sequence set forth in SEQ ID NO: 2 and a CL comprising the amino acid sequence set forth in SEQ ID NO: 123;
(4) a heavy chain comprising a VH comprising the amino acid sequence set forth in SEQ ID NO: 7 and a CH comprising the amino acid sequence set forth in SEQ ID NO: 121, and a light chain comprising a VL comprising the amino acid sequence set forth in SEQ ID NO: 8 and a CL comprising the amino acid sequence set forth in SEQ ID NO: 144;
(5) a heavy chain comprising a VH comprising the amino acid sequence set forth in SEQ ID NO: 5 and a CH comprising the amino acid sequence set forth in SEQ ID NO: 121, and a light chain comprising a VL comprising the amino acid sequence set forth in SEQ ID NO: 6 and a CL comprising the amino acid sequence set forth in SEQ ID NO: 144;
(6) a heavy chain comprising a VH comprising the amino acid sequence set forth in SEQ ID NO: 11 and a CH comprising the amino acid sequence set forth in SEQ ID NO: 121, and a light chain comprising a VL comprising the amino acid sequence set forth in SEQ ID NO: 12 and a CL comprising the amino acid sequence set forth in SEQ ID NO: 123;
(7) a heavy chain comprising a VH comprising the amino acid sequence set forth in SEQ ID NO: 9 and a CH comprising the amino acid sequence set forth in SEQ ID NO: 121, and a light chain comprising a VL comprising the amino acid sequence set forth in SEQ ID NO: 10 and a CL comprising the amino acid sequence set forth in SEQ ID NO: 123;
(8) a heavy chain comprising a VH comprising the amino acid sequence set forth in SEQ ID NO: 15 and a CH comprising the amino acid sequence set forth in SEQ ID NO: 121, and a light chain comprising a VL comprising the amino acid sequence set forth in SEQ ID NO: 16 and a CL comprising the amino acid sequence set forth in SEQ ID NO: 123; or
(9) a heavy chain comprising a VH comprising the amino acid sequence set forth in SEQ ID NO: 13 and a CH comprising the amino acid sequence set forth in SEQ ID NO: 121, and a light chain comprising a VL comprising the amino acid sequence set forth in SEQ ID NO: 14 and a CL comprising the amino acid sequence set forth in SEQ ID NO: 123.

23. (canceled)

24. The antibody-drug conjugate of claim 1, wherein the antibody or the antigen-binding fragment thereof comprises:
(1) a heavy chain comprising the amino acid sequence set forth in SEQ ID NO: 126, and a light chain comprising the amino acid sequence set forth in SEQ ID NO: 125;
(2) a heavy chain comprising the amino acid sequence set forth in SEQ ID NO: 124, and a light chain comprising the amino acid sequence set forth in SEQ ID NO: 125;
(3) a heavy chain comprising the amino acid sequence set forth in SEQ ID NO: 127, and a light chain comprising the amino acid sequence set forth in SEQ ID NO: 128;
(4) a heavy chain comprising the amino acid sequence set forth in SEQ ID NO: 129, and a light chain comprising the amino acid sequence set forth in SEQ ID NO: 130;
(5) a heavy chain comprising the amino acid sequence set forth in SEQ ID NO: 131, and a light chain comprising the amino acid sequence set forth in SEQ ID NO: 132;
(6) a heavy chain comprising the amino acid sequence set forth in SEQ ID NO: 133, and a light chain comprising the amino acid sequence set forth in SEQ ID NO: 134;
(7) a heavy chain comprising the amino acid sequence set forth in SEQ ID NO: 135, and a light chain comprising the amino acid sequence set forth in SEQ ID NO: 136;
(8) a heavy chain comprising the amino acid sequence set forth in SEQ ID NO: 137, and a light chain comprising the amino acid sequence set forth in SEQ ID NO: 138; or
(9) a heavy chain comprising the amino acid sequence set forth in SEQ ID NO: 139, and a light chain comprising the amino acid sequence set forth in SEQ ID NO: 140.

25. The antibody-drug conjugate of claim 24, wherein the heavy chain comprising the amino acid sequence set forth in any one of SEQ ID NOs: 126, 124, 127, 129, 131, 133, 135, 137, 139 has an N-terminal glutamine subjected to cyclization to form pyroglutamic acid or pyroglutamate; and/or the heavy chain comprising the amino acid sequence set forth in any one of SEQ ID NOs: 126, 124, 127, 129, 131, 133, 135, 137, 139 lacks a C-terminal lysine.

26. The antibody-drug conjugate of claim 1, wherein the antibody comprises:
a heavy chain comprising the amino acid sequence set forth in SEQ ID NO: 141 and a light chain comprising the amino acid sequence set forth in SEQ ID NO: 125;
a heavy chain comprising the amino acid sequence set forth in SEQ ID NO: 142 and a light chain comprising the amino acid sequence set forth in SEQ ID NO: 125; or
a heavy chain comprising the amino acid sequence set forth in SEQ ID NO: 143 and a light chain comprising the amino acid sequence set forth in SEQ ID NO: 125.

27. The antibody-drug conjugate of claim 1, wherein M is 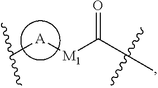
and wherein ring A is a 5-6-membered aliphatic heterocyclic ring or a 5-20-membered aromatic ring system, and the aliphatic heterocyclic ring and the aromatic ring system are optionally substituted with one or more groups selected from oxo (=O), halogen, cyano, amino, carboxyl, sulfhydryl, and C₁₋₆alkyl; M₁ is selected from a single bond, C₁₋₂₀alkylene, C₂₋₂₀alkenylene, and C₂₋₂₀alkynylene.

28.-31. (canceled)

32. The antibody-drug conjugate of claim 1, wherein L is selected from the structures comprising one or more of the following: C₁₋₆alkylene, —N(R′)—, carbonyl, —O—, natural amino acids or unnatural amino acids and analogs thereof (such as Ala, Arg, Asn, Asp, Cit, Cys, Gln, Glu, Gly, His, Ile, Leu, Lys, Met, Phe, Pro, Ser, Thr, Trp, Tyr, Val, Lys (COCH₂CH₂(OCH₂CH₂)ᵣOCH₃)), and short peptides consisting of amino acids (such as Ala-Ala, Ala-Lys, Ala-Lys(Ac), Ala-Pro, Gly-Glu, Gly-Gly, Phe-Lys, Phe-Lys(Ac), Val-Ala, Val-Lys, Val-Lys(Ac), Val-Cit, Ala-Ala-Ala, Ala-Ala-Asn, Leu-Ala-Glu, Gly-Gly-Arg, Gly-Glu-Gly, Gly-Gly-Gly, Gly-Ser-Lys, Glu-Val-Ala, Glu-Val-Cit, Ser-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly), 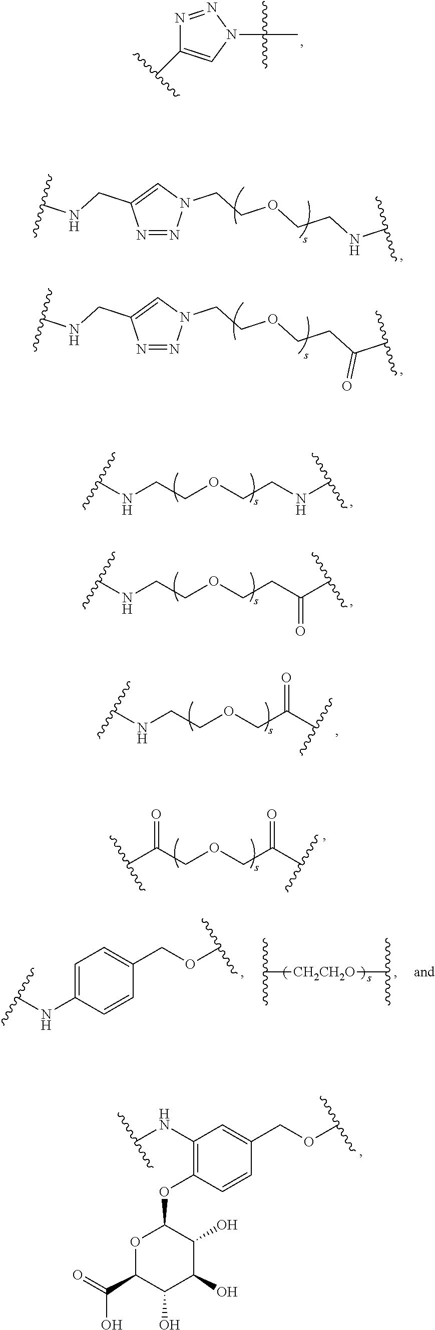
or a combination thereof; wherein R′ represents hydrogen, C₁₋₆alkyl, or a polyethylene glycol fragment containing 1-10 EO units; s is selected from integers of 1-20, for integers of 1-15, 1-10, 1-8, 1-6, 1-4, 1-2, and 3-6, for example, s is 1, 2, 3, 4, 5, 6, 7, 8, 9, 10, 11, 12, 13, 14, or 15.

33. (canceled)

34. The antibody-drug conjugate of claim 1, wherein L is selected from the following structures:

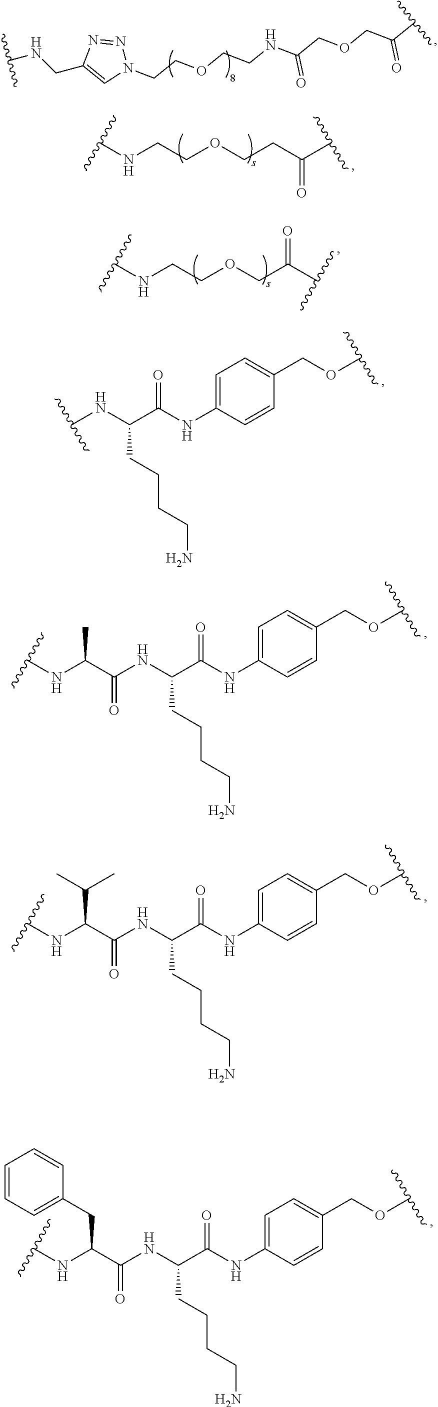

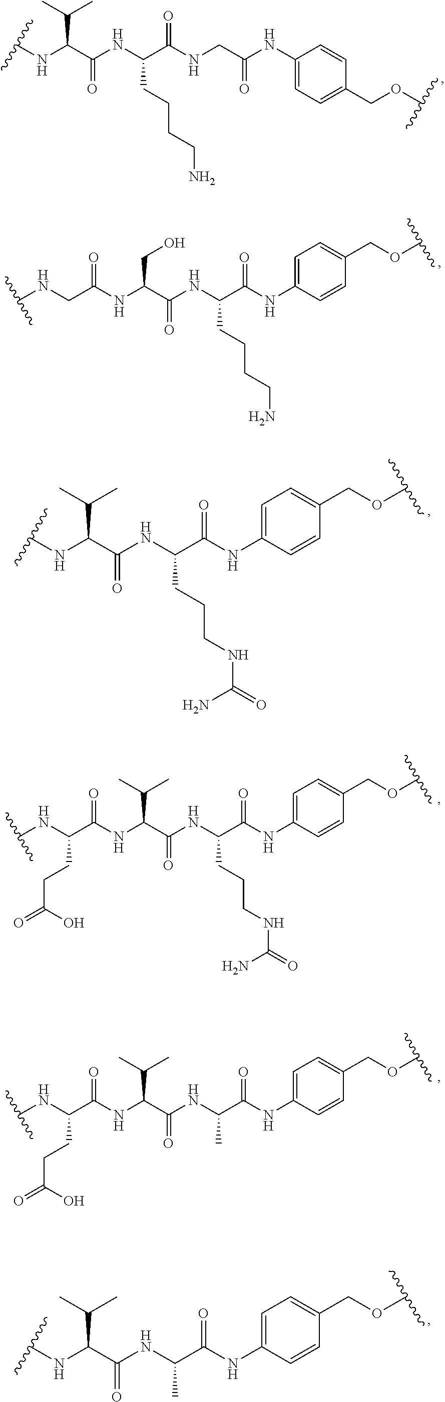 [SMILES: *N[C@H](C(=O)N[C@@H](CCCCN)C(=O)NCC(=O)Nc1ccc(CO*)cc1)C(C)C ; *NCC(=O)N[C@@H](CO)C(=O)N[C@@H](CCCCN)C(=O)Nc1ccc(CO*)cc1 ; *N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](C)C(=O)Nc1ccc(CO*)cc1)C(C)C ; *N[C@H](C(=O)N[C@@H](C)C(=O)Nc1ccc(CO*)cc1)C(C)C]

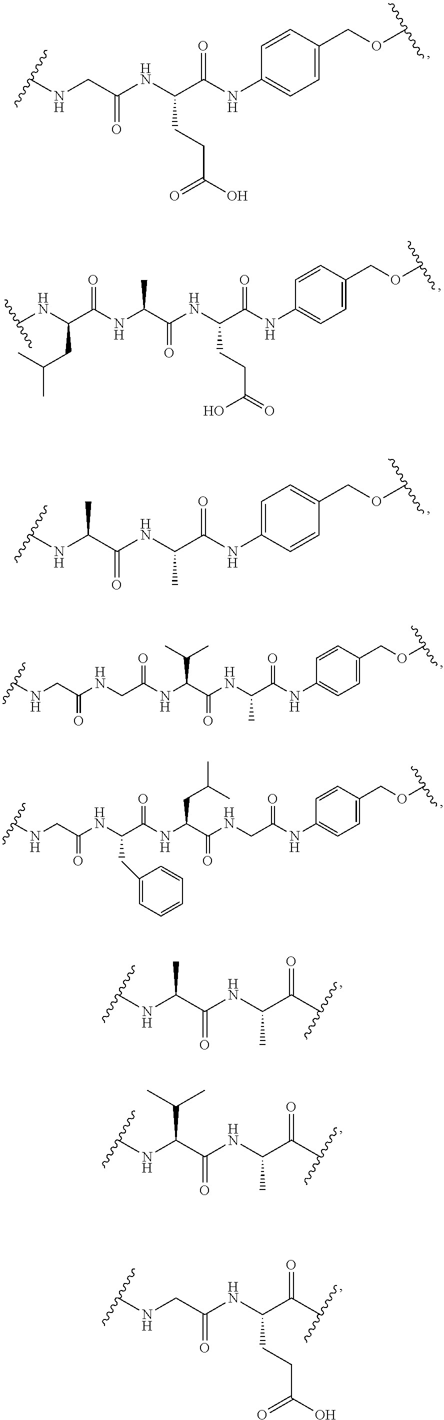

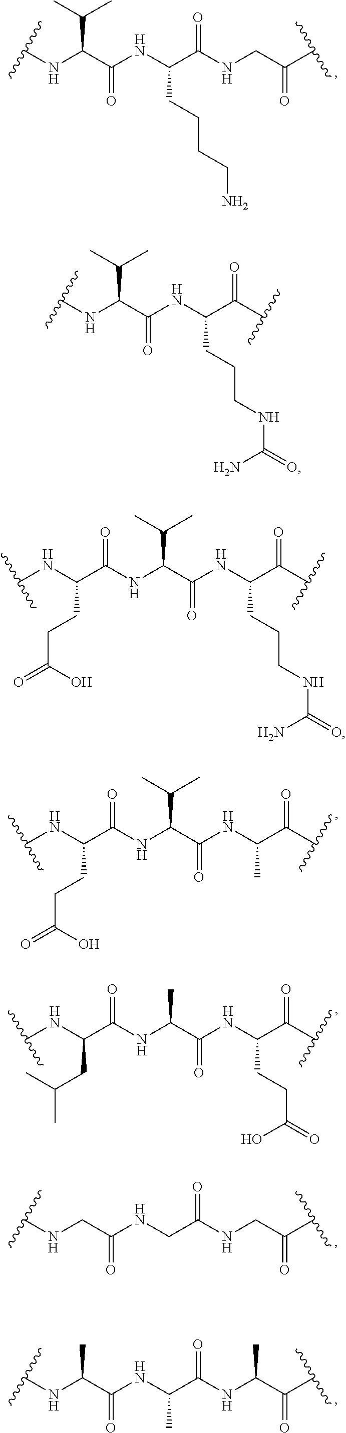

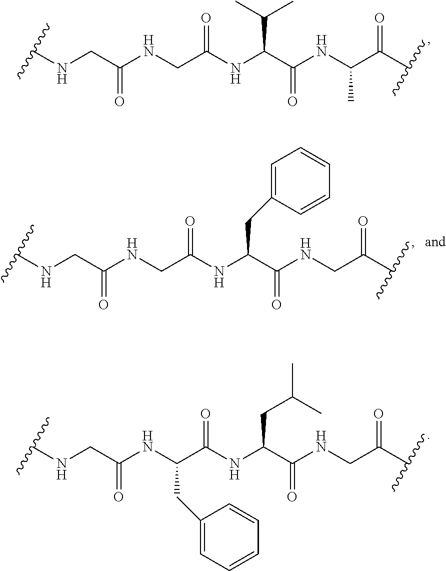 [SMILES: *NCC(=O)NCC(=O)N[C@H](C(=O)N[C@@H](C)C(*)=O)C(C)C ; *NCC(=O)NCC(=O)N[C@@H](Cc1ccccc1)C(=O)NCC(*)=O ; *NCC(=O)N[C@@H](Cc1ccccc1)C(=O)N[C@@H](CC(C)C)C(=O)NCC(*)=O]

35.-37. (canceled)

38. The antibody-drug conjugate of claim 1, wherein E is a single bond, —NH—CH₂—, —NH—CH₂—O—CH₂—CO—,

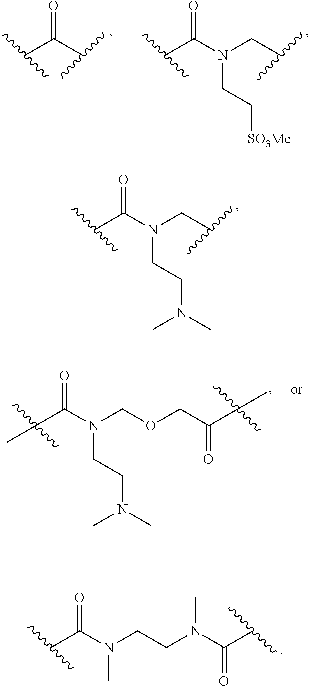

39.-41. (canceled)

42. The antibody-drug conjugate of claim 1, wherein 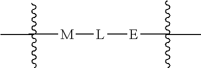
is selected from the following structures:

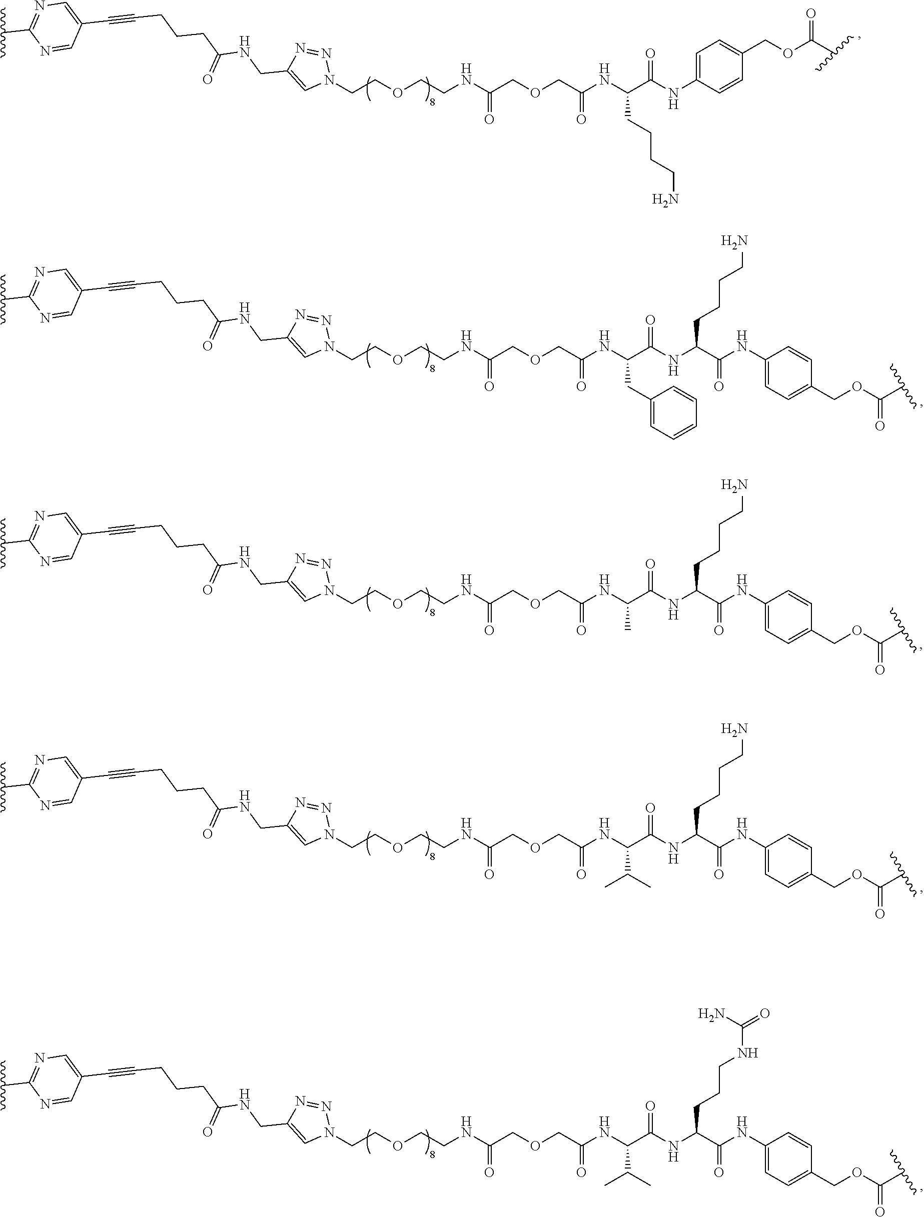

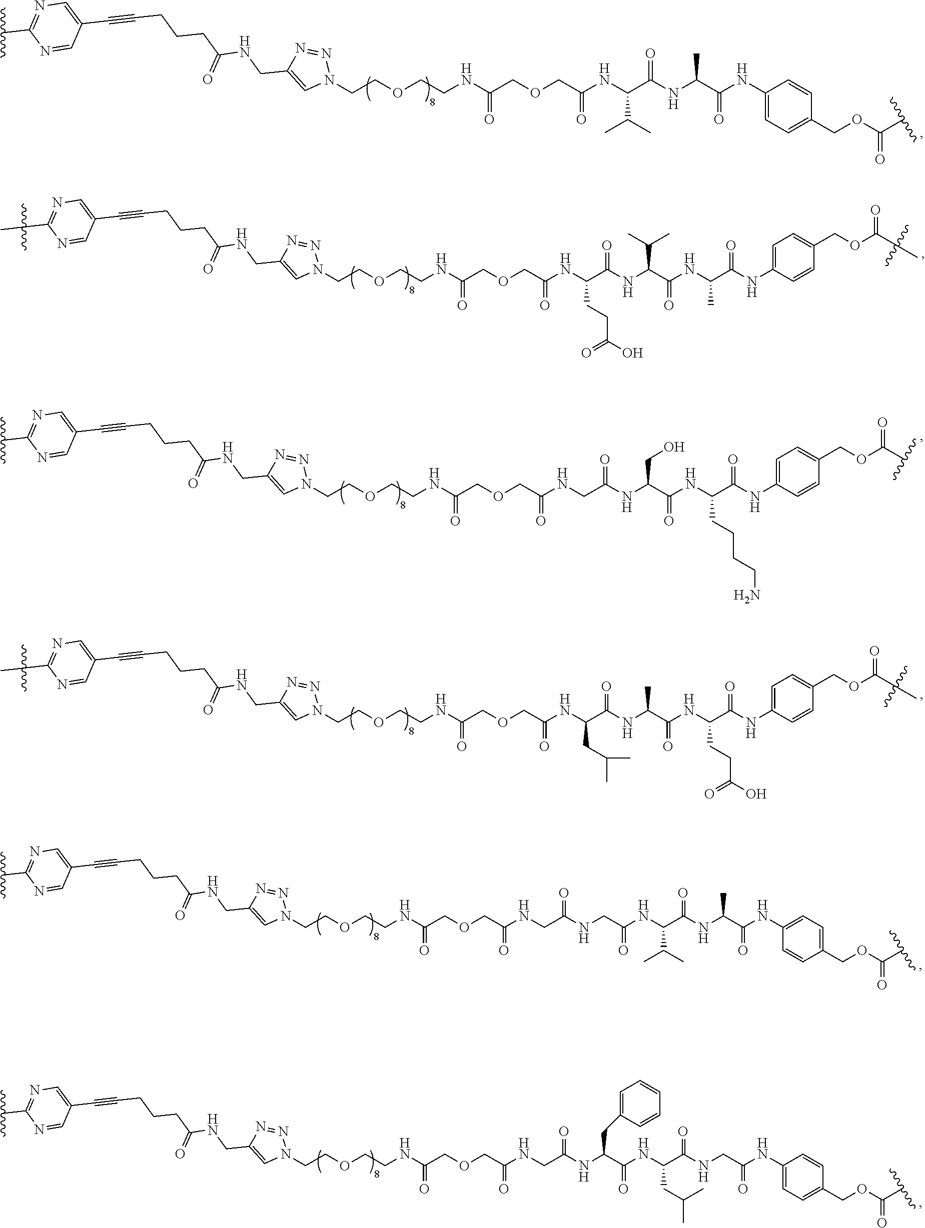

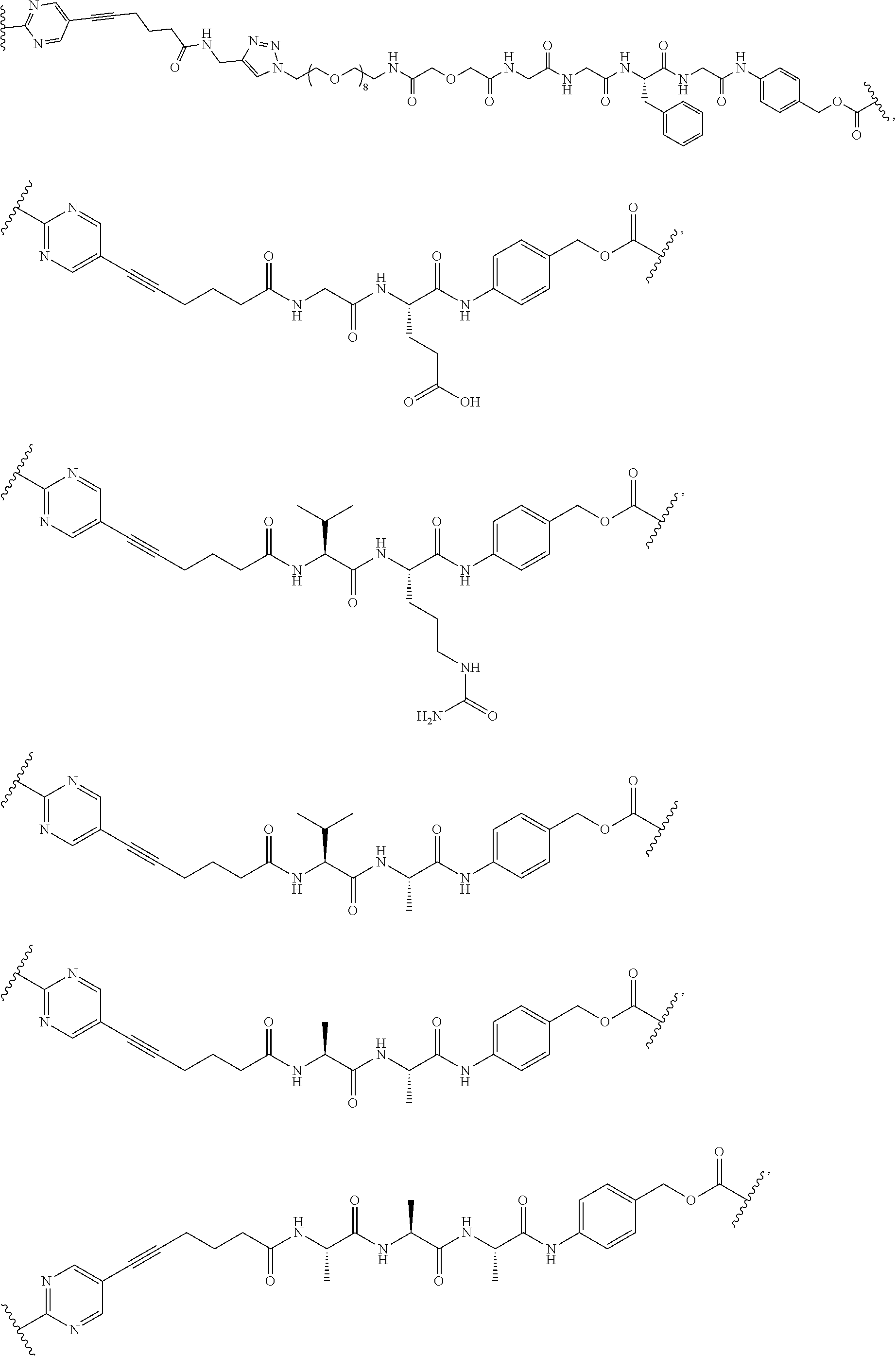

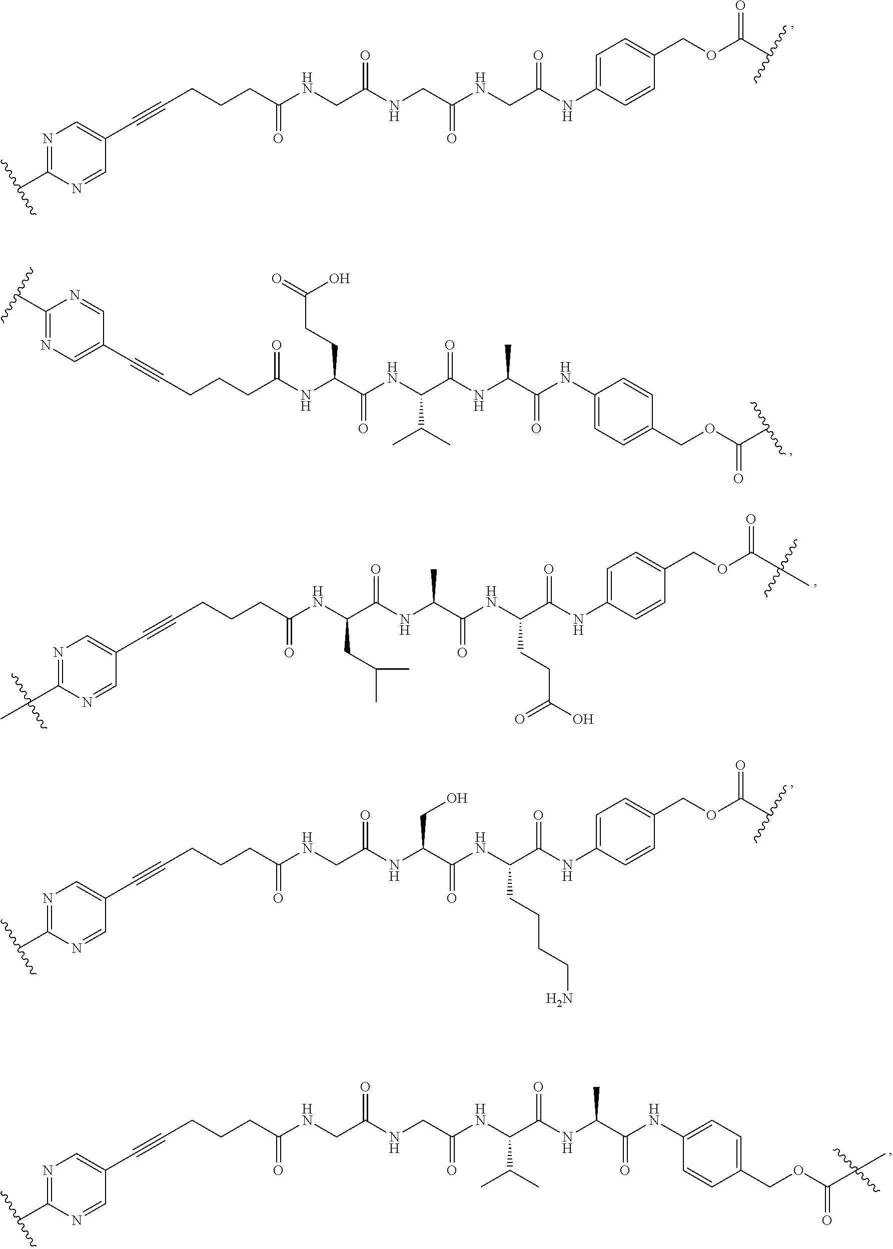

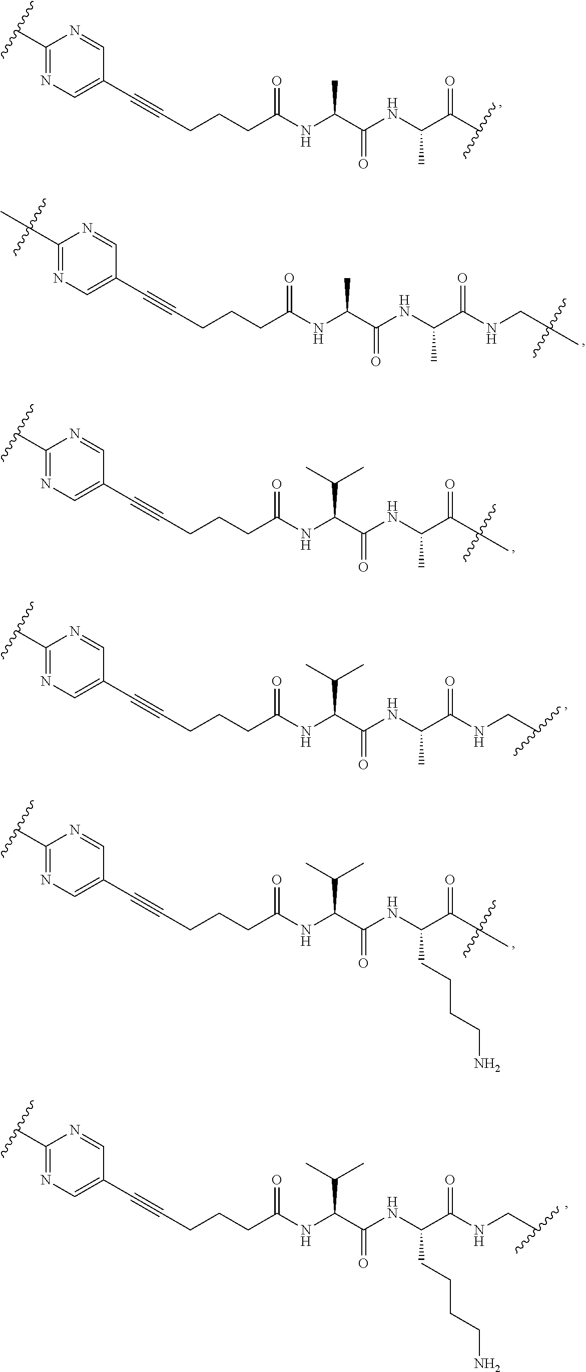 [SMILES: *C(=O)[C@H](C)NC(=O)[C@H](C)NC(=O)CCCC#Cc1cnc(*)nc1 ; *CNC(=O)[C@H](C)NC(=O)[C@H](C)NC(=O)CCCC#Cc1cnc(*)nc1 ; *C(=O)[C@H](C)NC(=O)[C@@H](NC(=O)CCCC#Cc1cnc(*)nc1)C(C)C ; *CNC(=O)[C@H](C)NC(=O)[C@@H](NC(=O)CCCC#Cc1cnc(*)nc1)C(C)C ; *C(=O)[C@H](CCCCN)NC(=O)[C@@H](NC(=O)CCCC#Cc1cnc(*)nc1)C(C)C ; *CNC(=O)[C@H](CCCCN)NC(=O)[C@@H](NC(=O)CCCC#Cc1cnc(*)nc1)C(C)C]

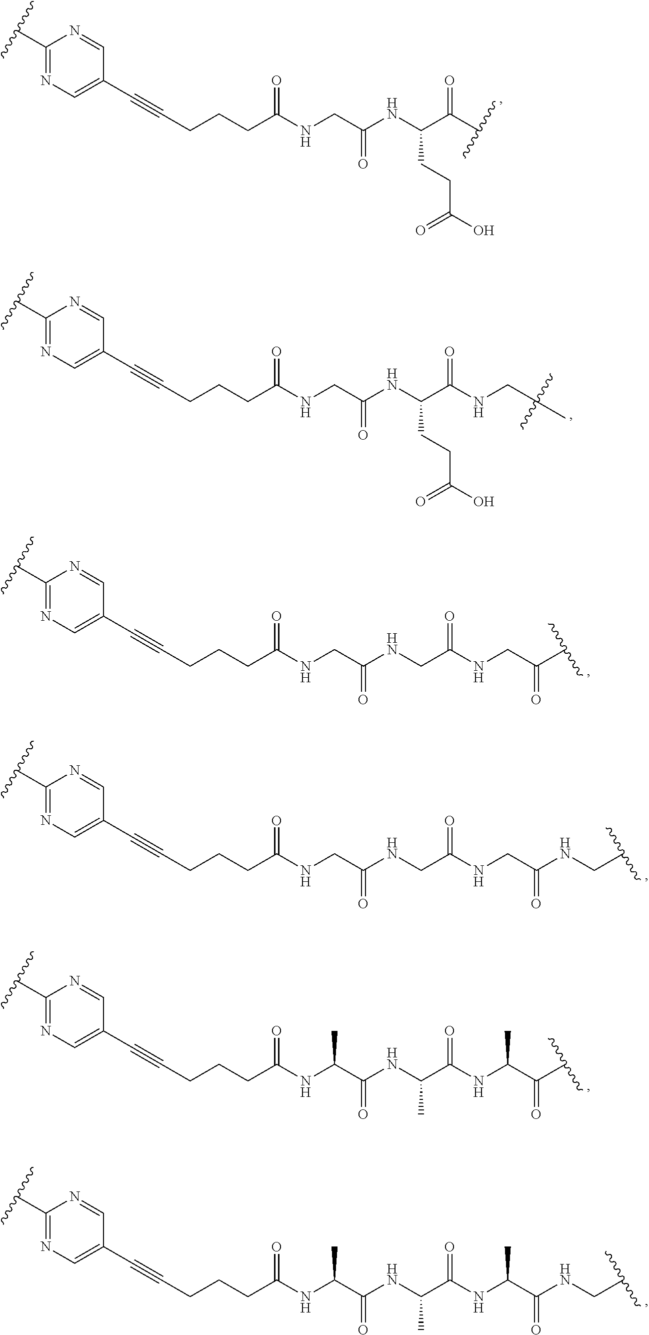 [SMILES: *C(=O)[C@H](CCC(=O)O)NC(=O)CNC(=O)CCCC#Cc1cnc(*)nc1 ; *CNC(=O)[C@H](CCC(=O)O)NC(=O)CNC(=O)CCCC#Cc1cnc(*)nc1 ; *C(=O)CNC(=O)CNC(=O)CNC(=O)CCCC#Cc1cnc(*)nc1 ; *CNC(=O)CNC(=O)CNC(=O)CNC(=O)CCCC#Cc1cnc(*)nc1 ; *C(=O)[C@H](C)NC(=O)[C@H](C)NC(=O)[C@H](C)NC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1]

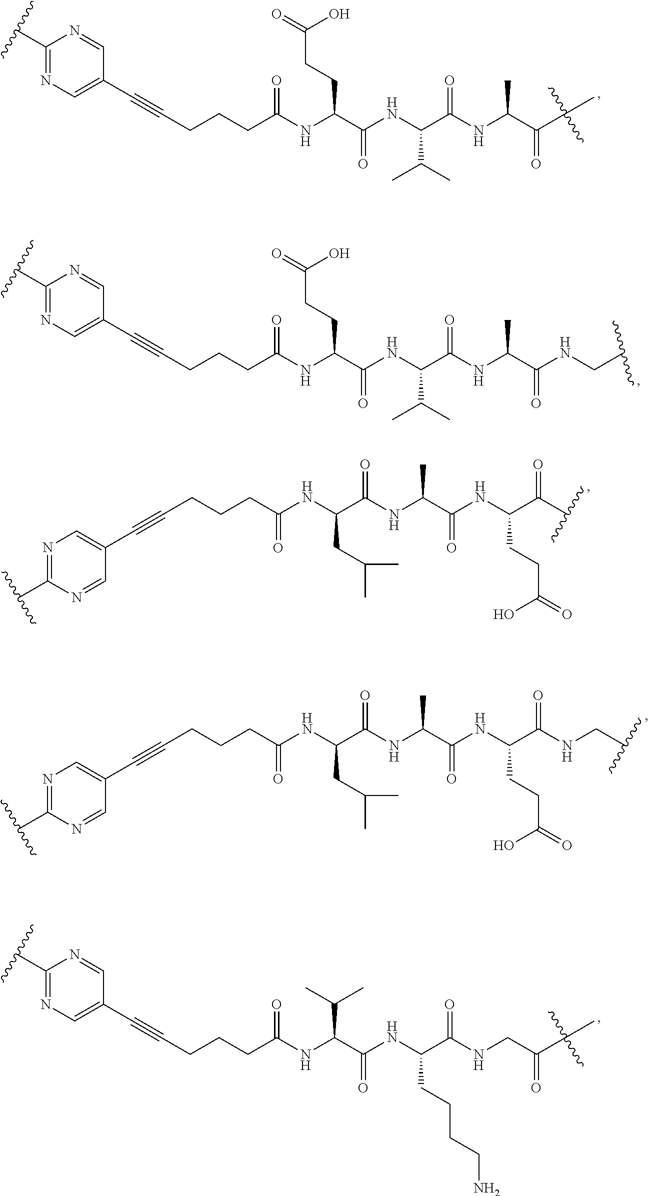 [SMILES: *C(=O)[C@H](C)NC(=O)[C@@H](NC(=O)[C@H](CCC(=O)O)NC(=O)CCCC#Cc1cnc(*)nc1)C(C)C ; *CNC(=O)[C@H](C)NC(=O)[C@@H](NC(=O)[C@H](CCC(=O)O)NC(=O)CCCC#Cc1cnc(*)nc1)C(C)C ; *C(=O)[C@H](CCC(=O)O)NC(=O)[C@H](C)NC(=O)[C@@H](CC(C)C)NC(=O)CCCC#Cc1cnc(*)nc1 ; *CNC(=O)[C@H](CCC(=O)O)NC(=O)[C@H](C)NC(=O)[C@@H](CC(C)C)NC(=O)CCCC#Cc1cnc(*)nc1 ; *C(=O)CNC(=O)[C@H](CCCCN)NC(=O)[C@@H](NC(=O)CCCC#Cc1cnc(*)nc1)C(C)C]

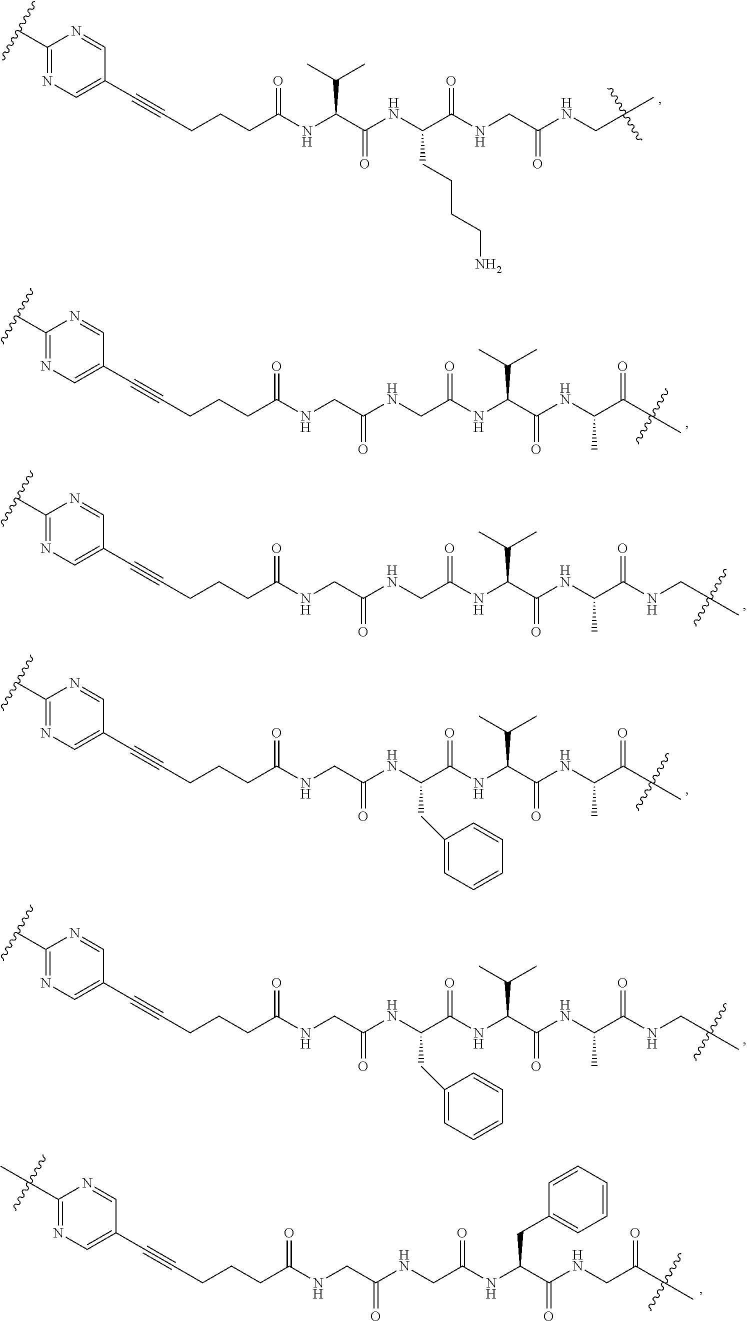 [SMILES: *CNC(=O)CNC(=O)[C@H](CCCCN)NC(=O)[C@@H](NC(=O)CCCC#Cc1cnc(*)nc1)C(C)C ; *C(=O)[C@H](C)NC(=O)[C@@H](NC(=O)CNC(=O)CNC(=O)CCCC#Cc1cnc(*)nc1)C(C)C ; *CNC(=O)[C@H](C)NC(=O)[C@@H](NC(=O)CNC(=O)CNC(=O)CCCC#Cc1cnc(*)nc1)C(C)C ; *C(=O)[C@H](C)NC(=O)[C@@H](NC(=O)[C@H](Cc1ccccc1)NC(=O)CNC(=O)CCCC#Cc1cnc(*)nc1)C(C)C ; *CNC(=O)[C@H](C)NC(=O)[C@@H](NC(=O)[C@H](Cc1ccccc1)NC(=O)CNC(=O)CCCC#Cc1cnc(*)nc1)C(C)C ; *C(=O)CNC(=O)[C@H](Cc1ccccc1)NC(=O)CNC(=O)CNC(=O)CCCC#Cc1cnc(*)nc1]

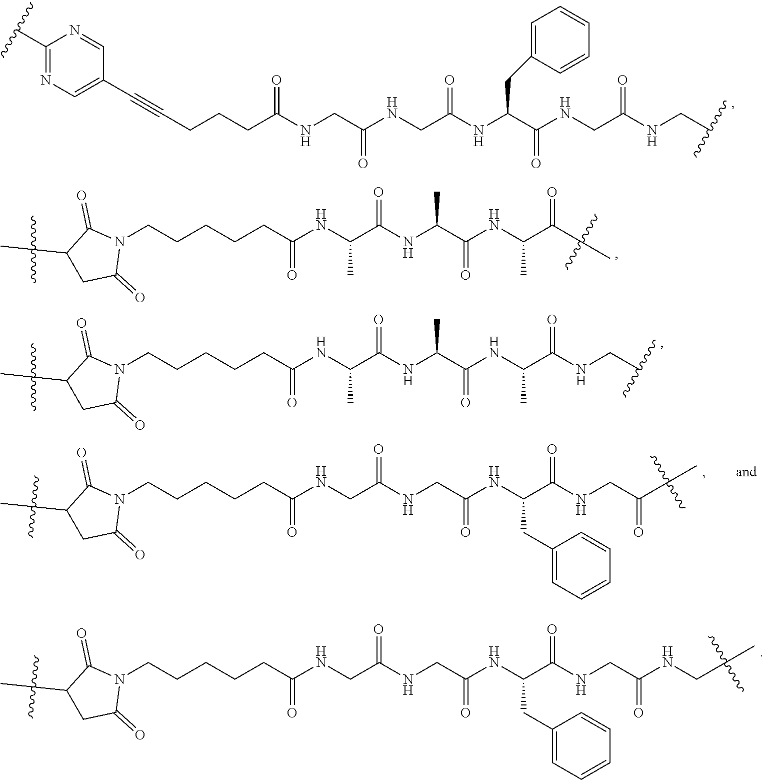

43.-44. (canceled)

45. The antibody-drug conjugate of claim 1, wherein the cytotoxic drug is selected from a tubulin inhibitor, a DNA intercalator, a DNA topoisomerase inhibitor, and an RNA polymerase inhibitor.

46.-49. (canceled)

50. The antibody-drug conjugate of claim 45, wherein the cytotoxic drug is selected from the following formula I and formula II: 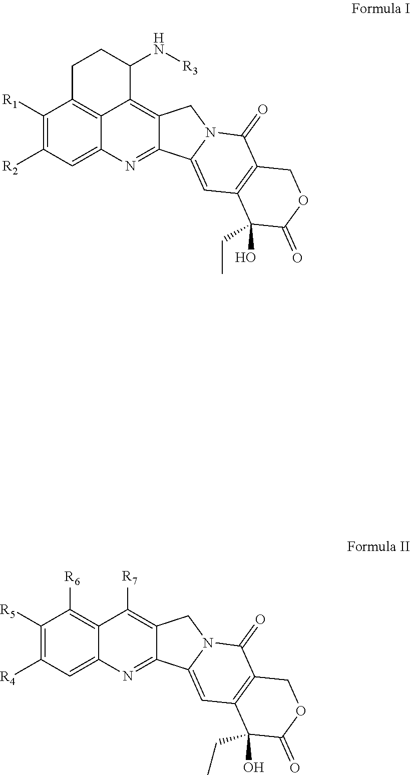
wherein R₁ and R₂ are each independently selected from C₁₋₆alkyl and halogen;
R₃ is selected from H and —CO—CH₂OH;
R₄ and R₅ are each independently selected from H, halogen, and hydroxy; or R₄ and R₅ are taken together with the carbon atom to which they are attached to form a 5-6-membered oxygen-containing heterocyclic ring;
R₆ is selected from hydrogen or —C₁₋₄alkylene-NRₐR_{b}; and
R₇ is selected from hydrogen, C₁₋₆ alkyl, —C₁₋₄ alkylene-NRₐR_{b}, —C₁₋₄ alkylene-SiRₐR_{b}R_{c}, —SiRₐR_{b}R_{c}, —C₁₋₄ alkylene=N—ORₐ; wherein, Rₐ, R_{b} and R_{c} are independently selected from H, C₁₋₆ alkyl, —SO₂—C₁₋₆ alkyl, and —CO—C₁₋₆ alkyl at each occurrence; wherein optionally Rₐ and R_{b} connected to the associated atoms form a 5-6 membered nitrogen containing heterocyclic ring.

51. The antibody-drug conjugate of claim 45, wherein the cytotoxic drug is selected from the following compounds:

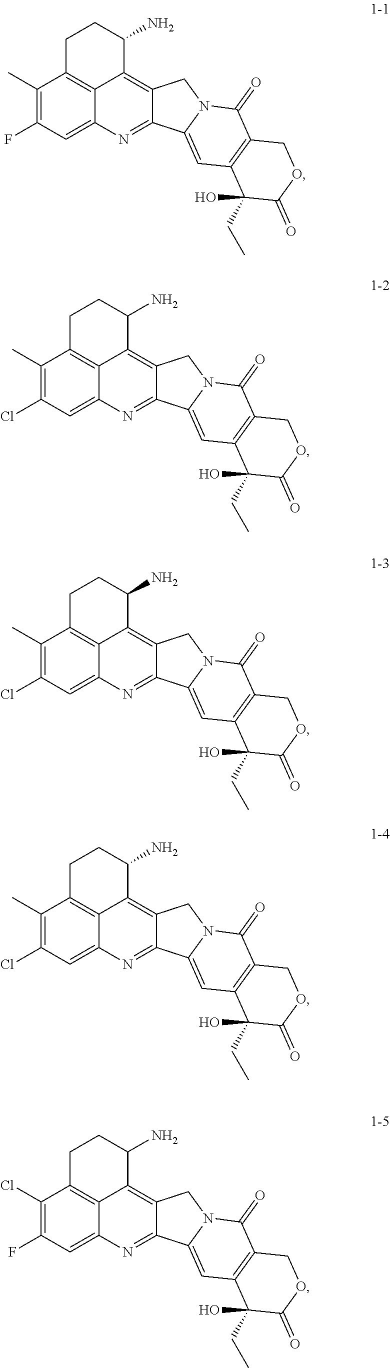 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

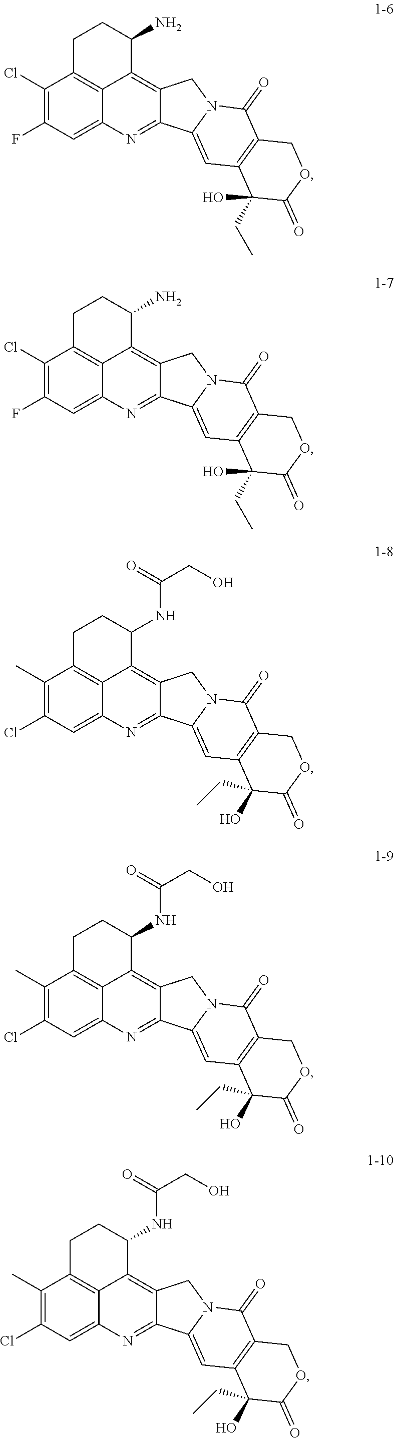 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

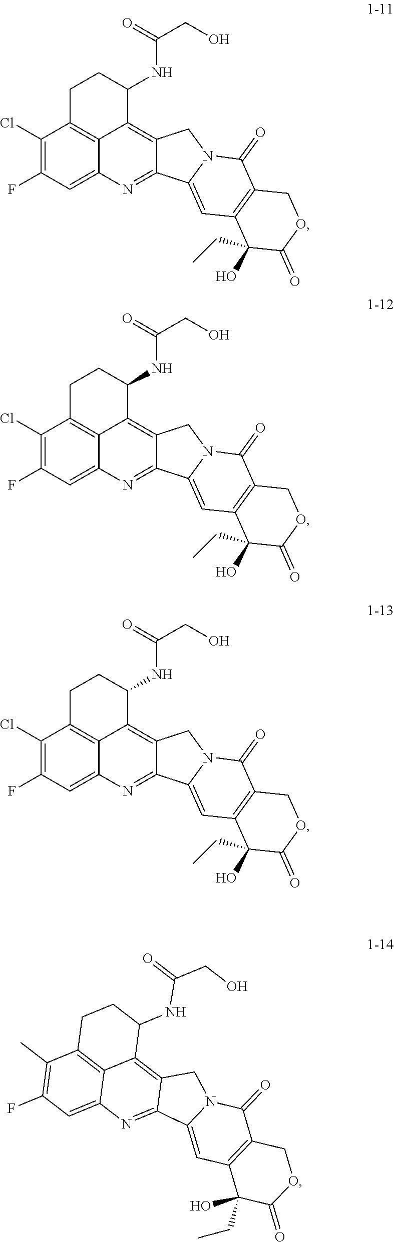 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2C(NC(=O)CO)CC3]

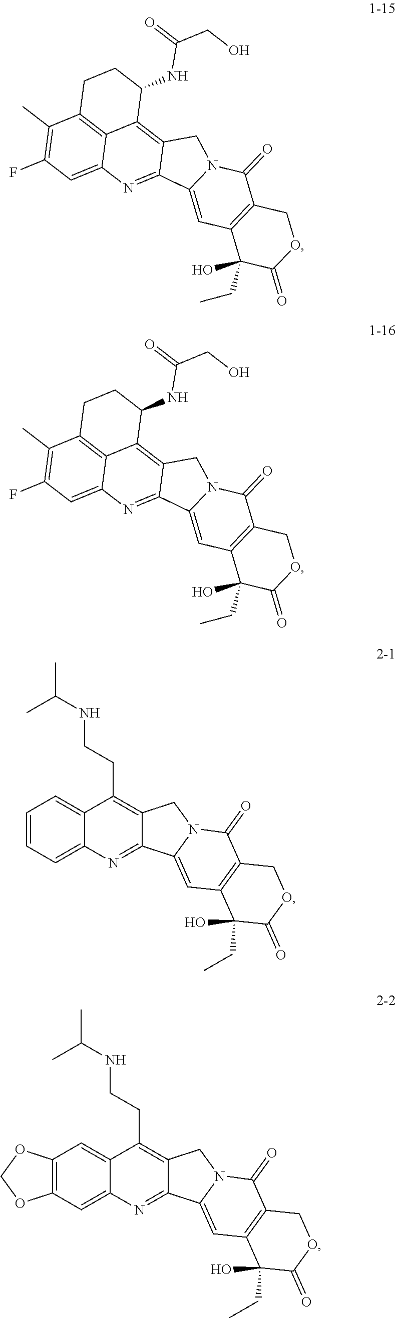 [SMILES: CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1cc(F)c(C)c3c1c2[C@H](NC(=O)CO)CC3 ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3]

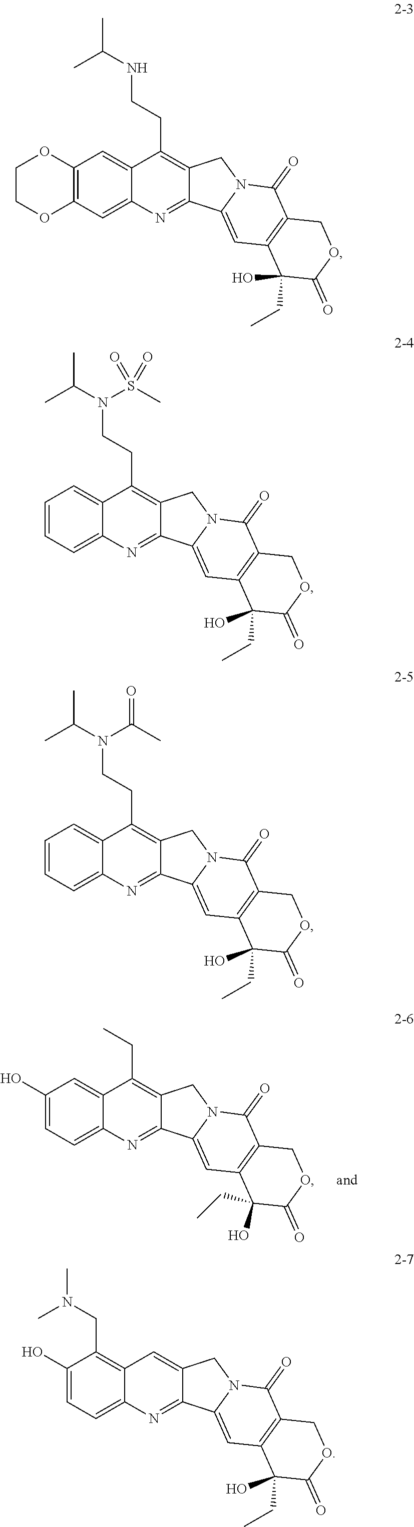 [SMILES: CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; "and" ; CC[C@@]1(O)C(=O)OCc2c1cc1n(c2=O)Cc2cc3c(CN(C)C)c(O)ccc3nc2-1]

52.-53. (canceled)

54. The antibody-drug conjugate of claim 1, wherein D is selected from the following structures:

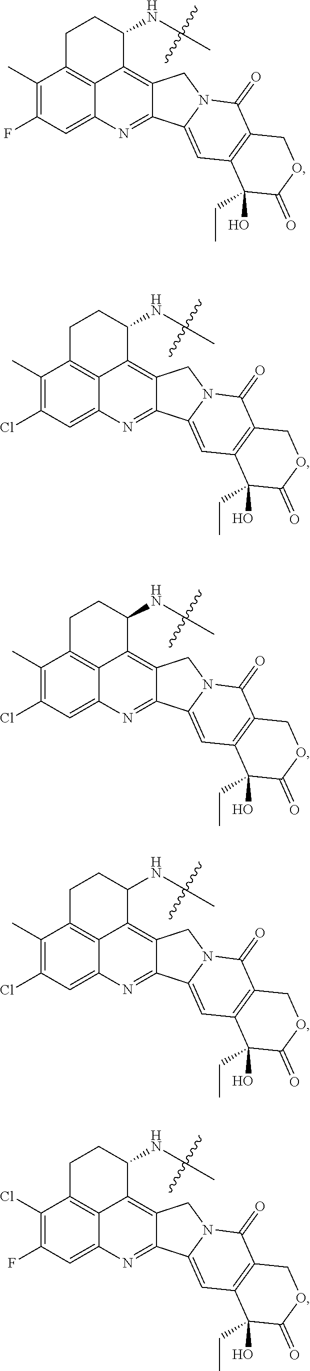 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC ; *N[C@H]1CCc2c(Cl)c(F)cc3nc4c(c1c23)Cn1c-4cc2c(c1=O)COC(=O)[C@]2(O)CC]

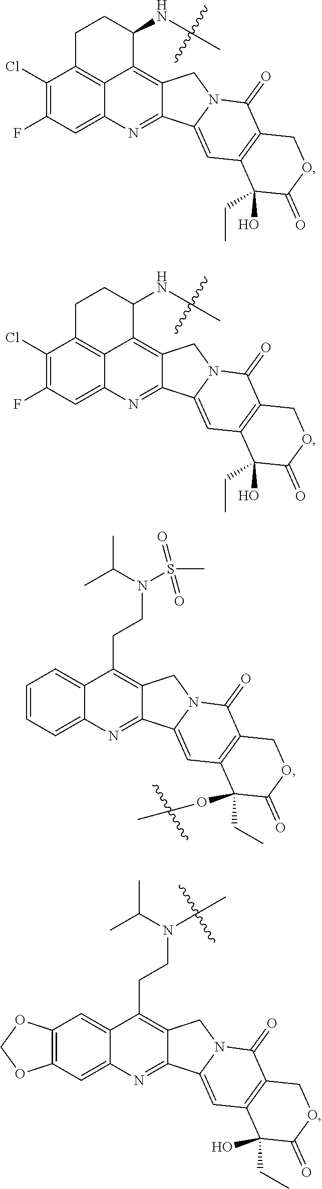 [SMILES: *N[C@@H]1CCc2c(Cl)c(F)cc3nc4c(c1c23)Cn1c-4cc2c(c1=O)COC(=O)[C@]2(O)CC ; *NC1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O ; *N(CCc1c2c(nc3cc4c(cc13)OCO4)-c1cc3c(c(=O)n1C2)COC(=O)[C@]3(O)CC)C(C)C]

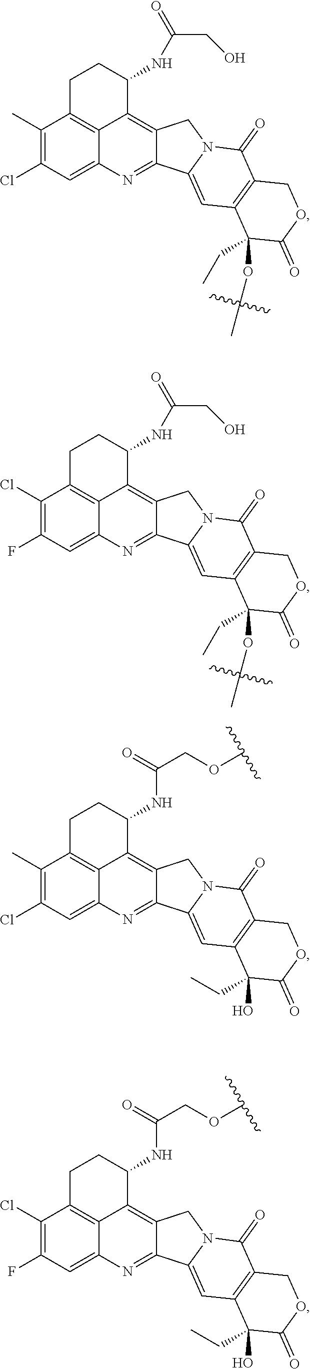 [SMILES: *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3 ; *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC]

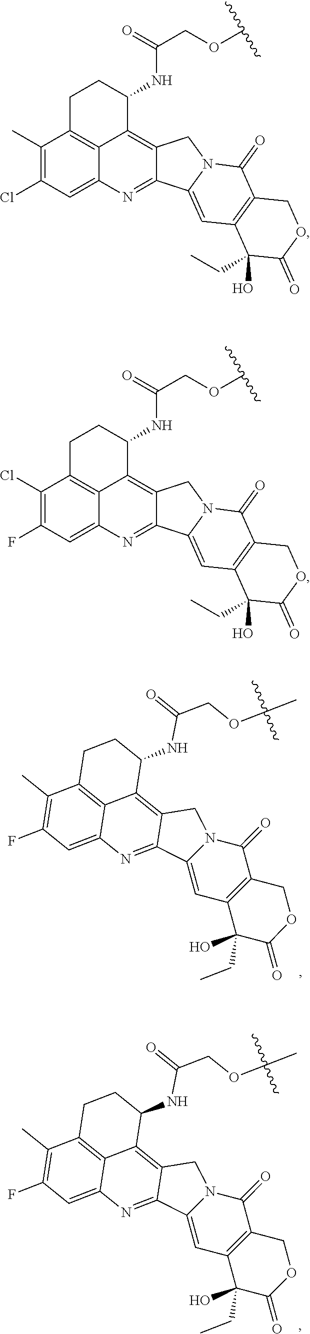 [SMILES: *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *OCC(=O)N[C@@H]1CCc2c(C)c(F)cc3nc4c(c1c23)Cn1c-4cc2c(c1=O)COC(=O)[C@]2(O)CC]

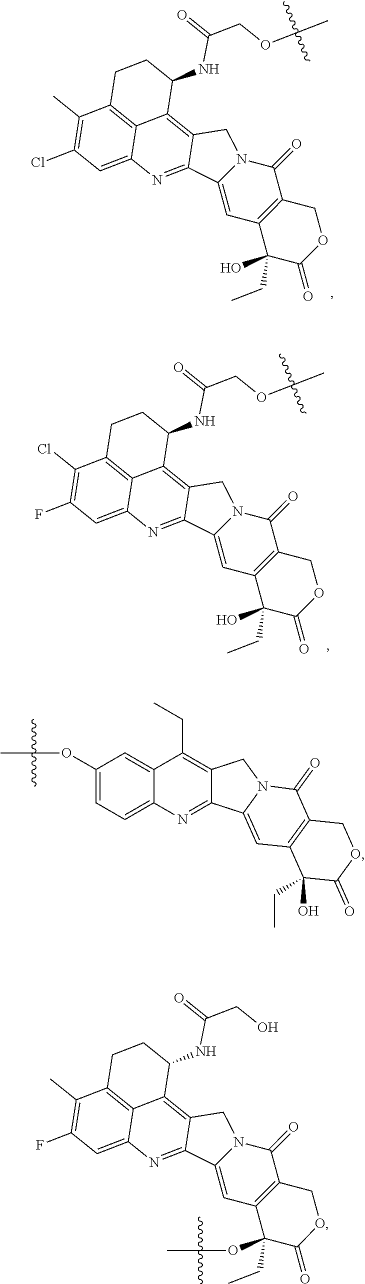 [SMILES: *OCC(=O)N[C@@H]1CCc2c(C)c(Cl)cc3nc4c(c1c23)Cn1c-4cc2c(c1=O)COC(=O)[C@]2(O)CC ; *OCC(=O)N[C@@H]1CCc2c(Cl)c(F)cc3nc4c(c1c23)Cn1c-4cc2c(c1=O)COC(=O)[C@]2(O)CC ; *Oc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1cc(F)c(C)c3c1c2[C@@H](NC(=O)CO)CC3]

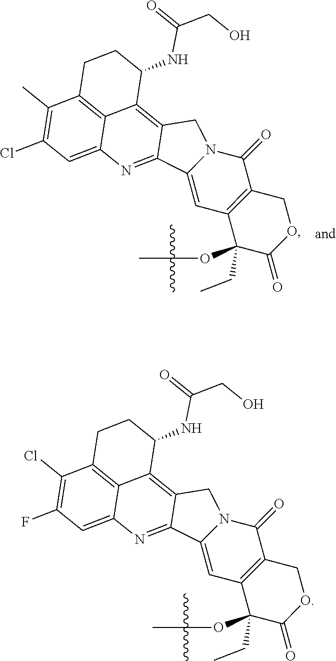 [SMILES: *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3]

55.-57. (canceled)

58. The antibody-drug conjugate of claim 1, wherein the antibody-drug conjugate is selected from ADC A-01 to ADC A-34 and ADC B-01 to ADC B-07 as shown below: 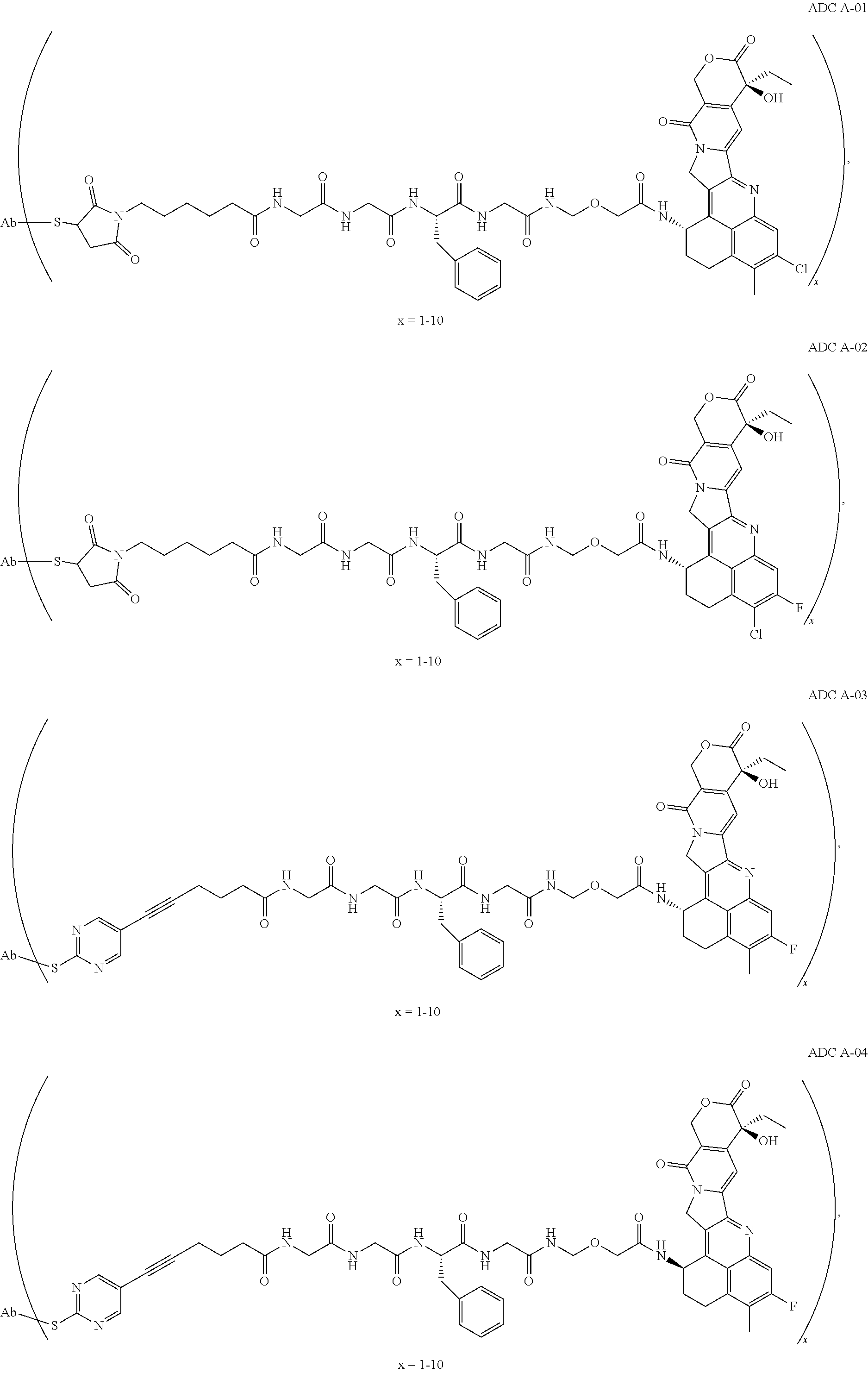 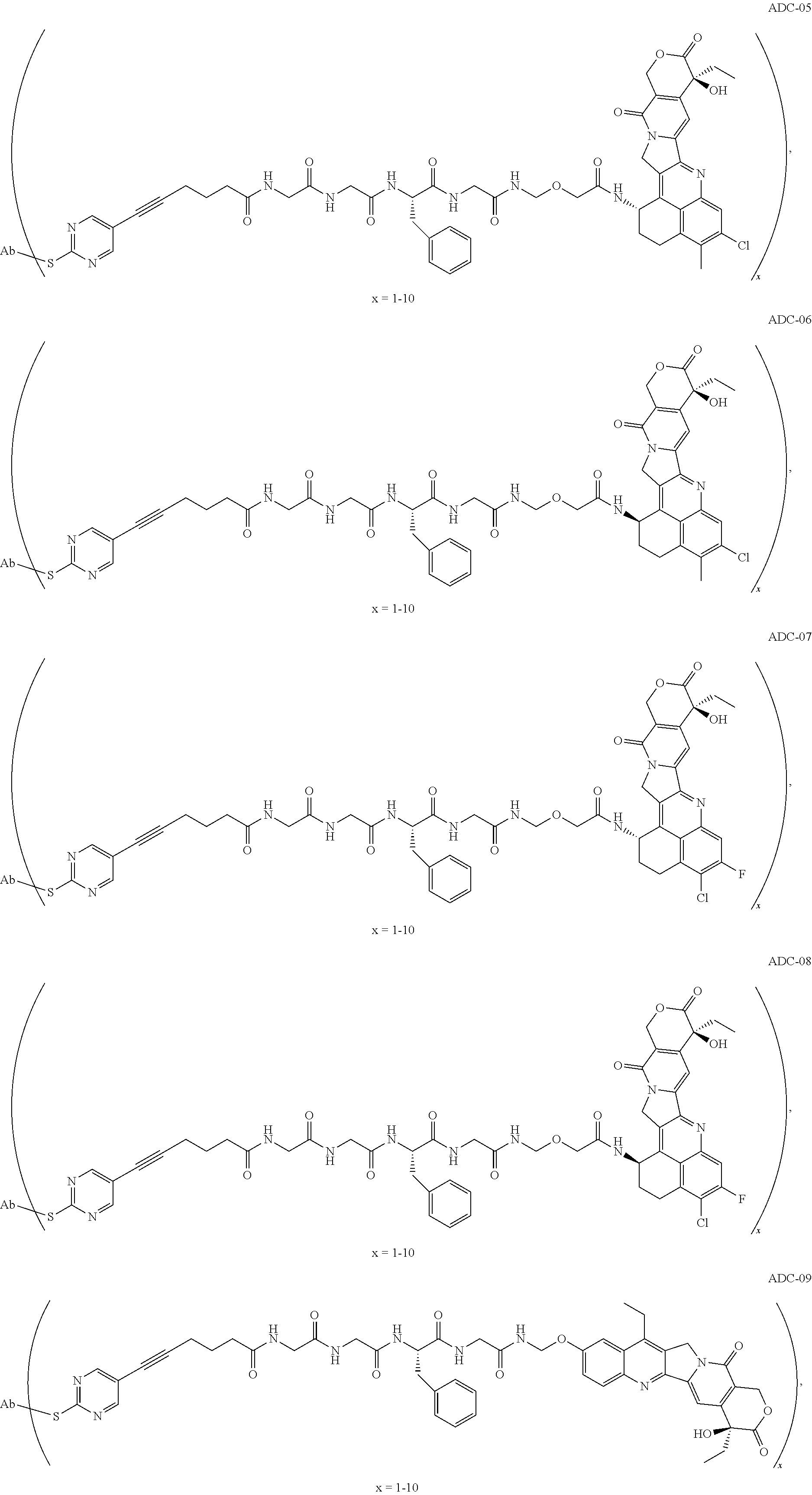 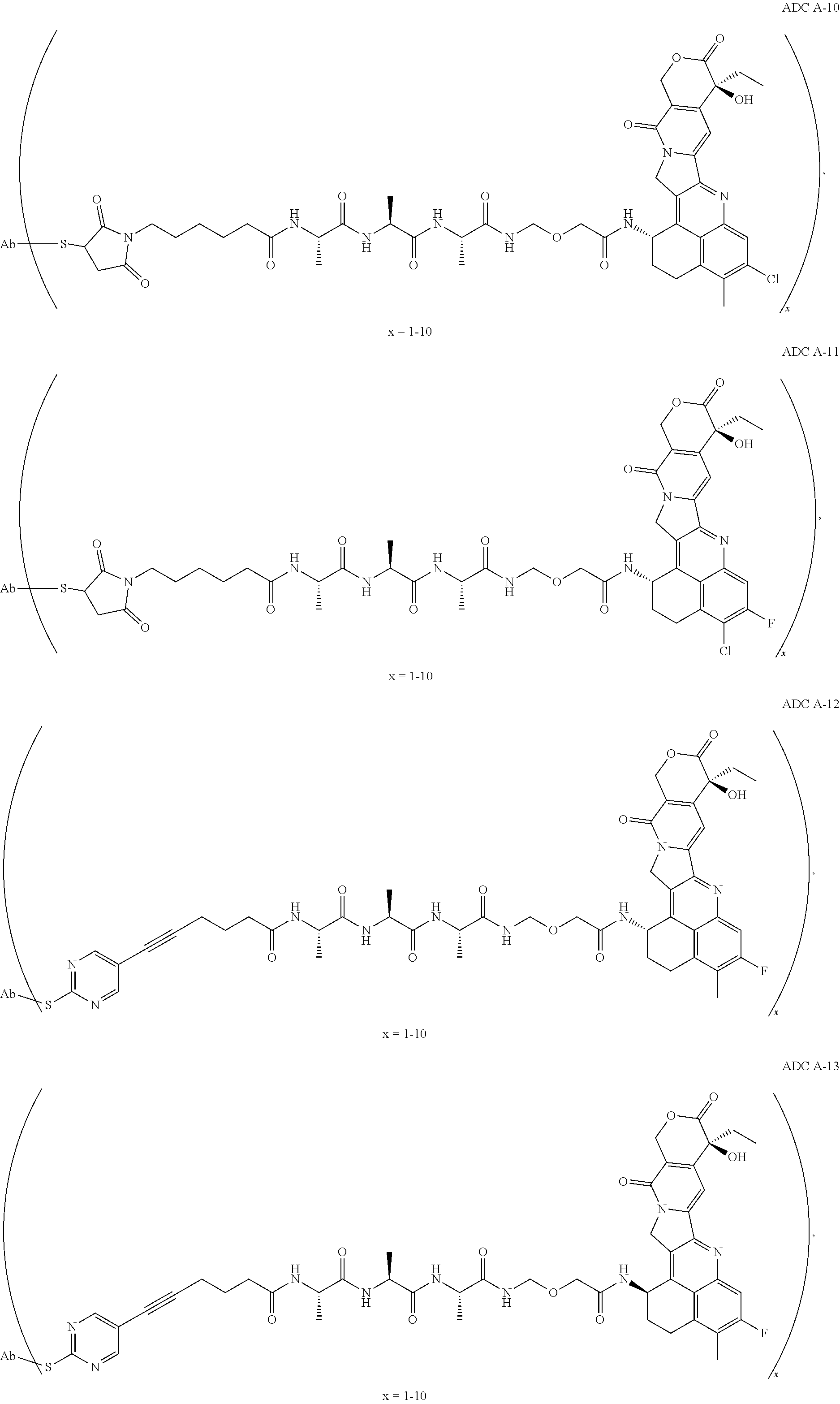 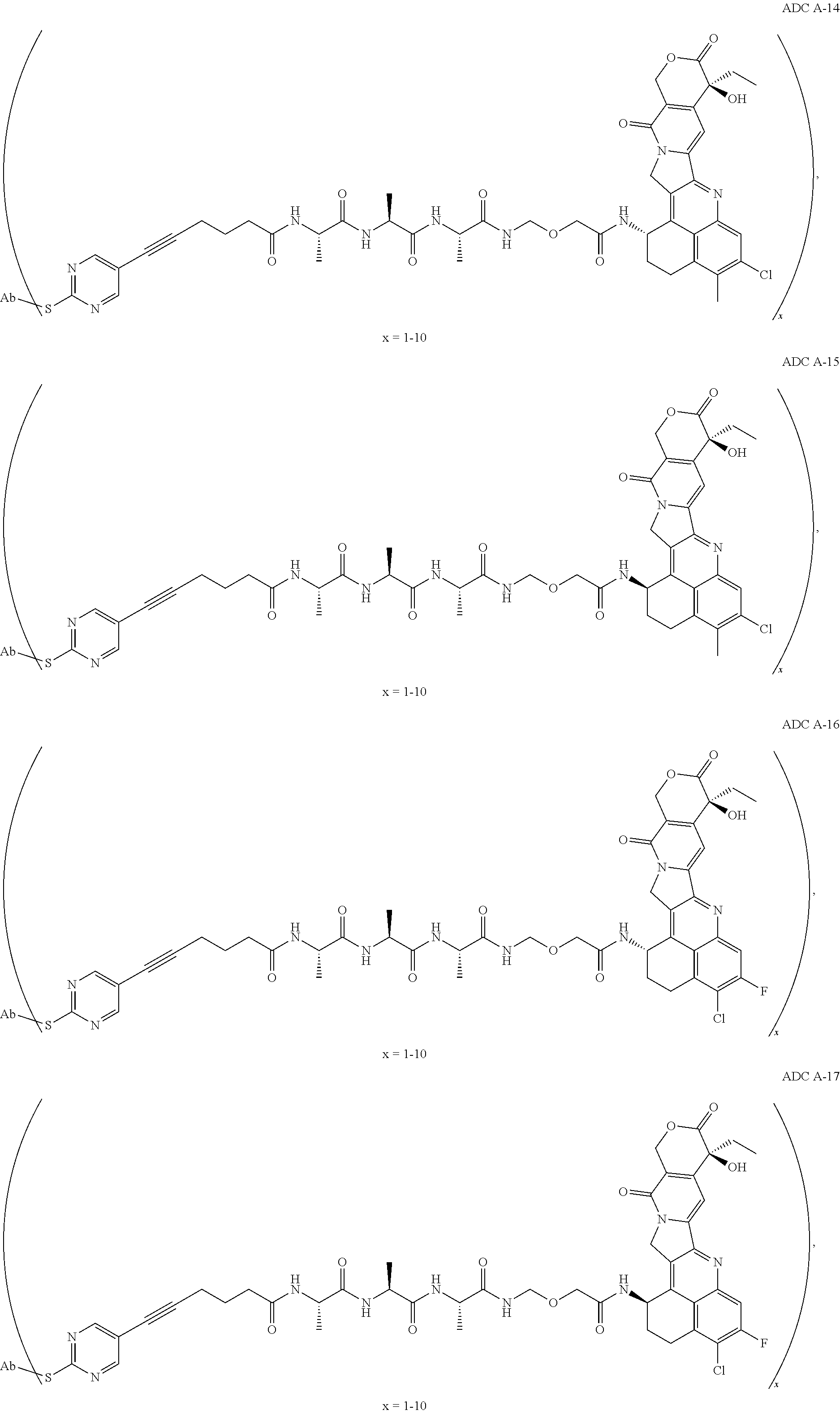 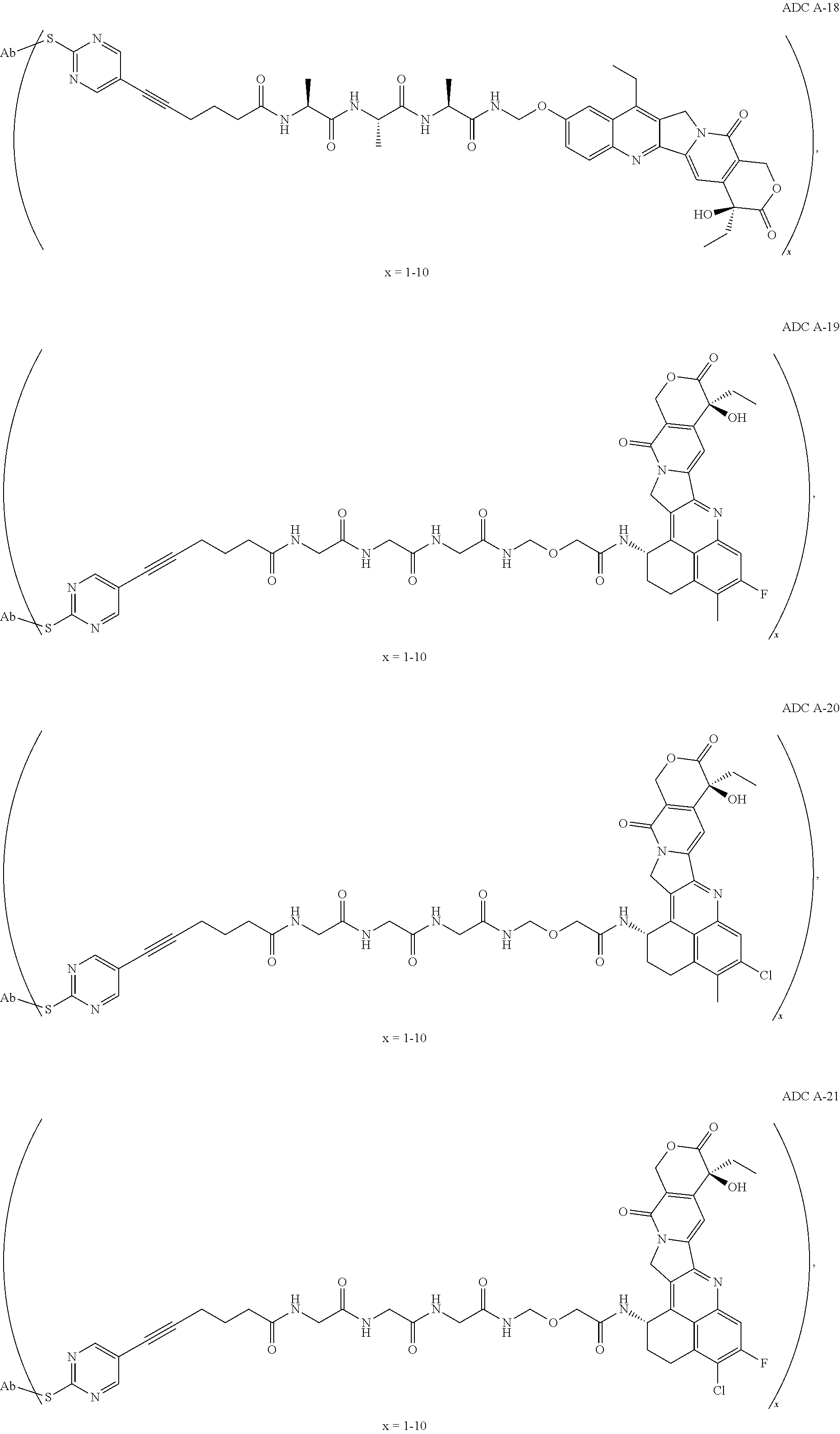 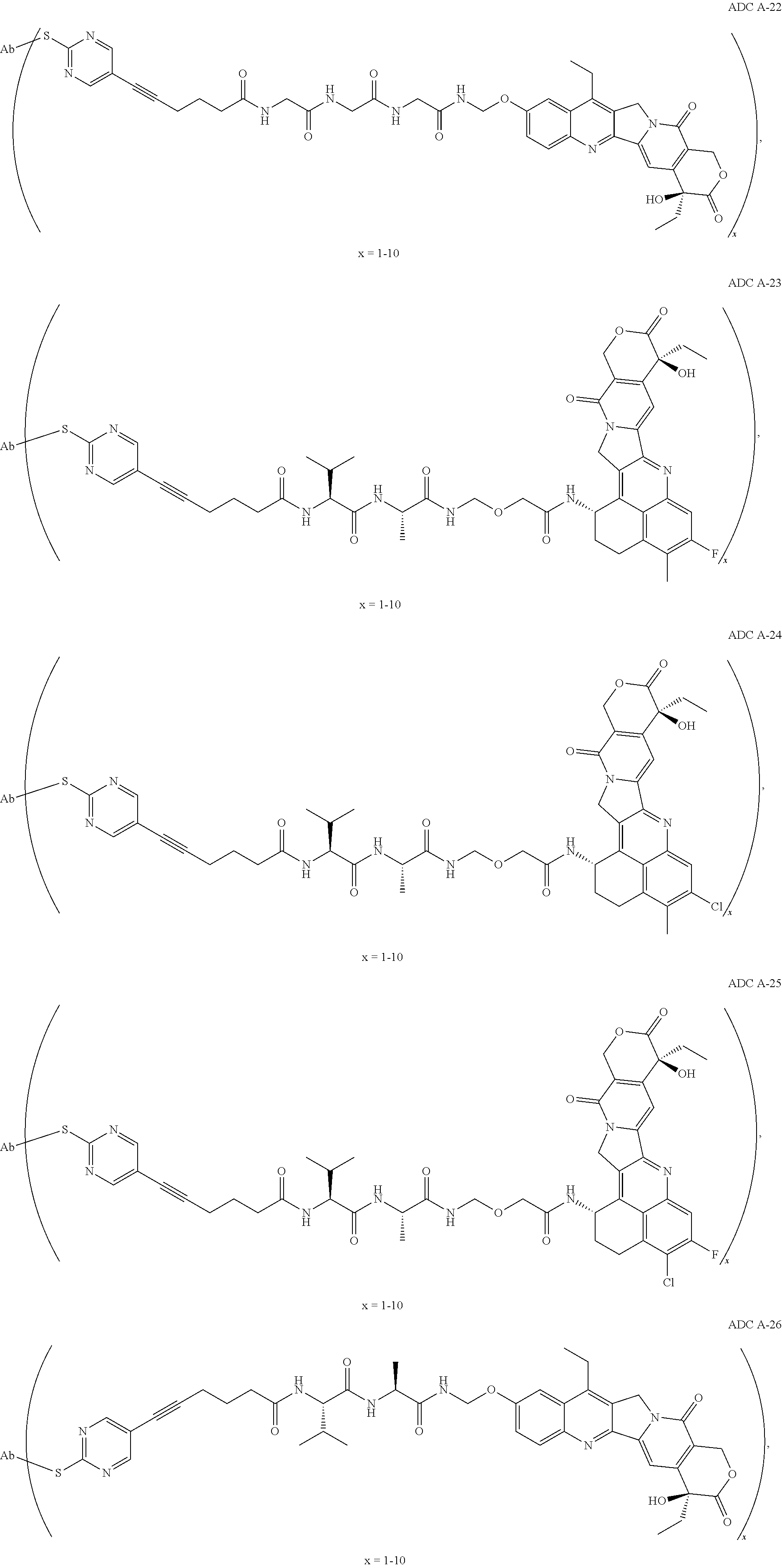 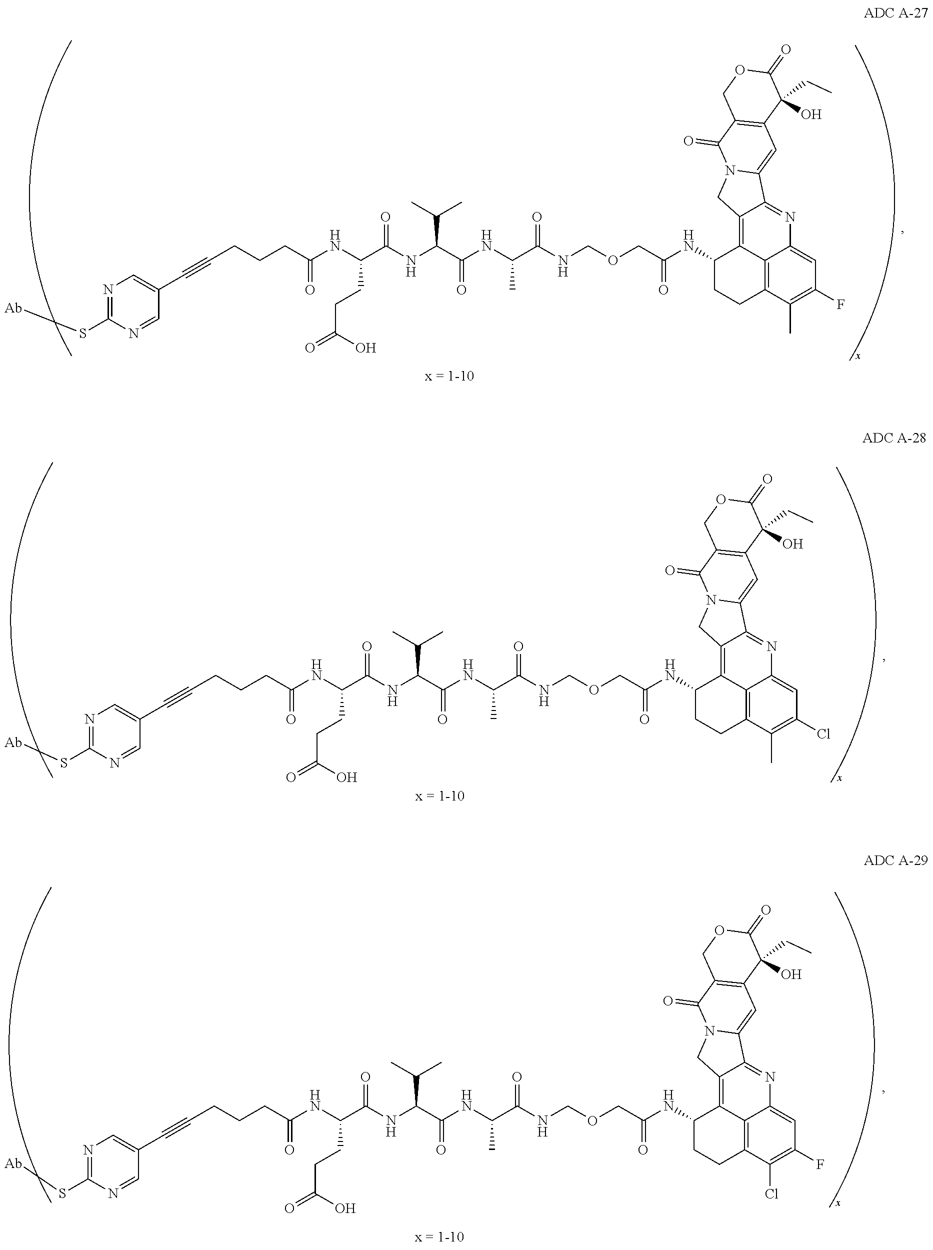 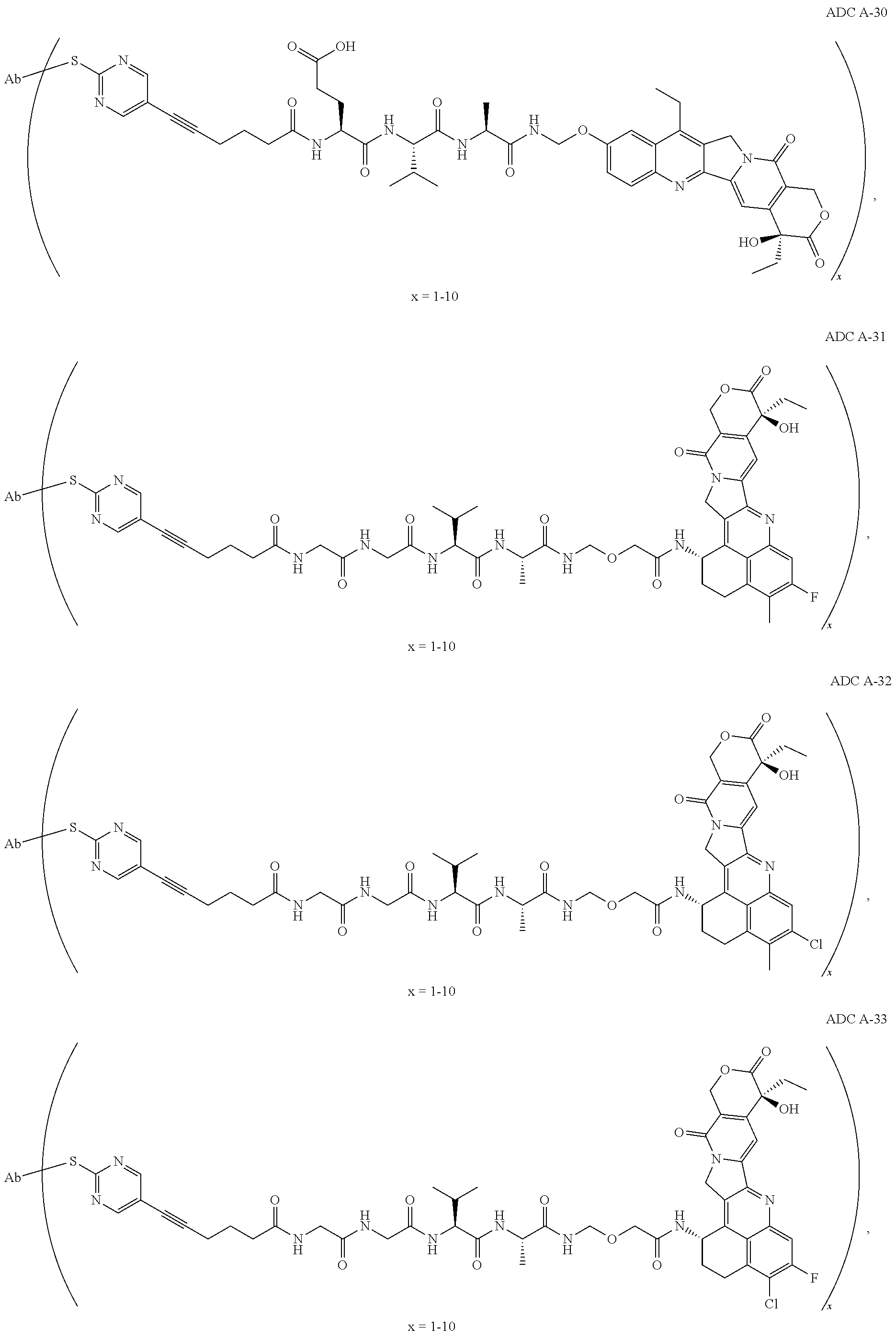 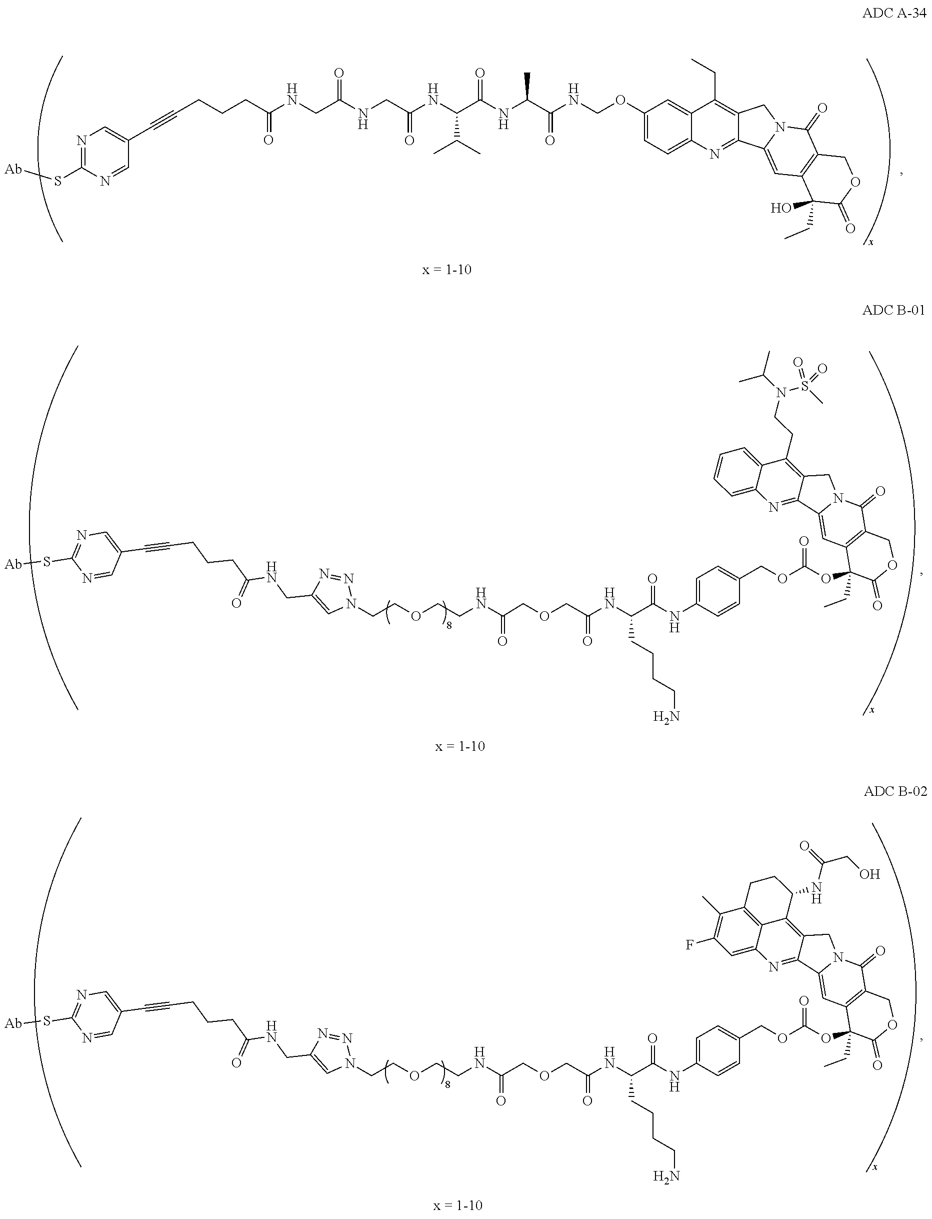 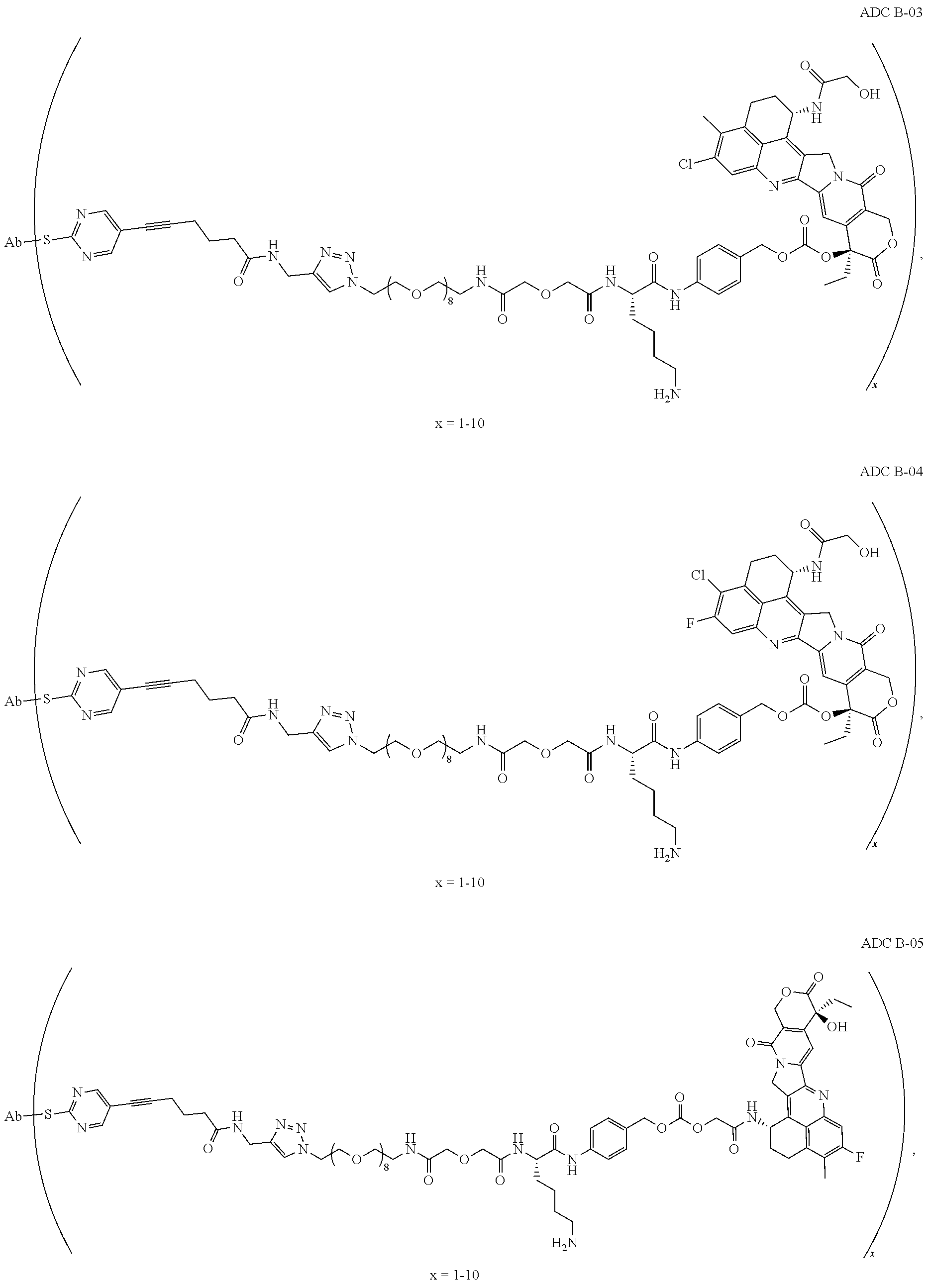 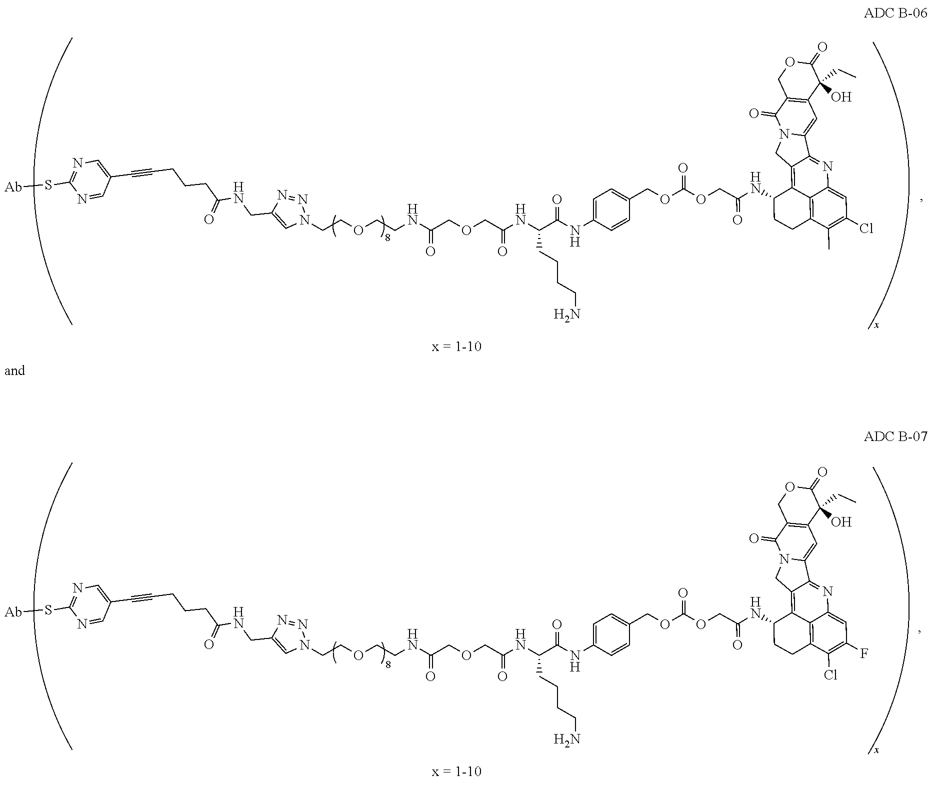
wherein Ab-(S— in each antibody-drug conjugate represents the antibody or the antigen-binding fragment thereof; and
wherein 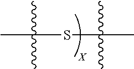 [SMILES: *S*]
represents a specific mode for connecting the sulfhydryl in the antibody or the antigen-binding fragment thereof with the linker.

59. A composition comprising one or more antibody-drug conjugates of claim 1, wherein a DAR value of the composition is 1, 2, 3, 4, 5, 6, 7, 8, 9, 10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10.

60.-61. (canceled)

62. A pharmaceutical composition comprising the antibody-drug conjugate of claim 1, and one or more pharmaceutical excipients.

63.-66. (canceled)

67. A method of inhibiting the activity of CDH3 in cells, comprising contacting the cells with the antibody-drug conjugate of claim 1, wherein the cells are cells that express CDH3.

68. A method of preventing or treating a CDH3-mediated disease or condition, comprising administering to a subject in need thereof a prophylactically or therapeutically effective amount of the antibody-drug conjugate of claim 1.

69.-75. (canceled)


SEQUENCE LISTING (151 sequences)

SEQ ID NO: 1 - protein, 116 aa, synthetic construct
  source 1..116: note = VH of Ab-B
QVQLQQPGTELVKPGASVKLSCQASGFTFTSYWMHWVKQRPGQGLAWIGNINLSYGNTNYNEKFKNKATLTVDKSSSTAYMQLSGLTSEDSAVYYCARGFSYFDYWGQGTTLTVSS

SEQ ID NO: 2 - protein, 107 aa, synthetic construct
  source 1..107: note = VL of Ab-B
DIVMTQSHKFMSTSLGDRVNITCKASQNVDTDVSWYQQKPGKSPKPLIYWASNRFTGVPDRFTGSGSGTDFTLTINNVQSEDLADYFCEQYSSYPFTFGTGTKLEIK

SEQ ID NO: 3 - protein, 116 aa, synthetic construct
  source 1..116: note = VH of Ab-BHZ\Ab-BHZm
QVQLVQSGAEVKKPGASVKVSCKASGFTFTSYWMHWVRQAPGQGLEWIGNIQLSYGNTNYAQKFQGRVTLTVDKSSSTAYMELSSLRSEDTAVYYCARGFSYFDYWGQGTLVTVSS

SEQ ID NO: 4 - protein, 107 aa, synthetic construct
  source 1..107: note = VL of Ab-BHZ\Ab-BHZm
DIQMTQSPSSLSASVGDRVTITCKASQNVDTDVSWYQQKPGKAPKSLIYWASNRFTGVPSRFSGSGSGTDFTLTISSLQPEDFATYFCEQYSSYPFTFGQGTKVEIK

SEQ ID NO: 5 - protein, 120 aa, synthetic construct
  source 1..120: note = VH of Ab-C
DVQLVESGGGLVQPGGSRKLSCAASGFTFTSFGMHWVRQTPEKGLEWVAYISSVSNIIYYADTVKGRFTISRDNPKNTLFLQMTSLRSEDTAMYYCARDGKYYYYTMDYWGQGTSVTVSS

SEQ ID NO: 6 - protein, 109 aa, synthetic construct
  source 1..109: note = VL of Ab-C
QAVVTQESALTTSPGETVTLTCRSSTGAVTTTNYANWVQEKPDHLFTGLIGGTNNRTPGVPARFSGSLIGDKAALTITGAQTEDEAVYFCALWYSNHWVFGGGTKLTVL

SEQ ID NO: 7 - protein, 120 aa, synthetic construct
  source 1..120: note = VH of Ab-CHZ
EVQLVESGGGLVQPGGSLRLSCAASGFTFTSFGMHWVRQAPGKGLEWVAYISSVSNIIYYADSVKGRFTISRDNAKNSLYLQMNSLRAEDTAVYYCARDGKYYYYTMDYWGQGTLVTVSS

SEQ ID NO: 8 - protein, 109 aa, synthetic construct
  source 1..109: note = VL of Ab-CHZ
QTVVTQEPSLTVSPGGTVTLTCRSSTGAVTTTNYANWVQQKPGQAFRALIGGTNQRTPWTPARFSGSLLGGKAALTLSGVQPEDEAEYYCALWYSNHWVFGGGTKLTVL

SEQ ID NO: 9 - protein, 118 aa, synthetic construct
  source 1..118: note = VH of Ab-D
QVQLQQSGAELMKPGASVKISCKATGYTFSSYWIEWVKQRPGHGLERIGEILPGSGNTNYNEKFKGKATFTADTSSNTAYMQLSSLTAEDSAVYYCARREYFGSRDYWGQGTTLTVSS

SEQ ID NO: 10 - protein, 107 aa, synthetic construct
  source 1..107: note = VL of Ab-D
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYAFGGGTKLEIK

SEQ ID NO: 11 - protein, 118 aa, synthetic construct
  source 1..118: note = VH of Ab-DHZ
QVQLVQSGAEVKKPGSSVKVSCKASGYTFSSYWIEWVRQAPGQGLERMGEILPGSGNTNYAQKFQGRVTITADKSTNTAYMELSSLRSEDTAVYYCARREYFGSRDYWGQGTLVTVSS

SEQ ID NO: 12 - protein, 107 aa, synthetic construct
  source 1..107: note = VL of Ab-DHZ
DIQMTQSPSSLSASVGDRVTITCQASQDISNYLNWYQQKPEGAVKLLIYYTSRLHTGVPSRFSGSGSGTDYTFTISSLQPEDIATYYCQQGNTLPYAFGGGTKVEIK

SEQ ID NO: 13 - protein, 118 aa, synthetic construct
  source 1..118: note = VH of Ab-A
QVQLQQSDAELVKPGVSVKISCKVSGSTFTDHTIHWMKQRPEQGLEWIGYIYPTDGSTQYNEKFRGKATLTADRSSNTAYMQLNSLTYEDSAVYFSSLRPLFNVIKHWGQGTSVTVSS

SEQ ID NO: 14 - protein, 113 aa, synthetic construct
  source 1..113: note = VL of Ab-A
DIVMTQSPSSLTVTAGEKVTMSCKSSQSLLDSGNHKNFLTWYQQKPGQPPKLLIFWASTRETGVPDRFTGSGSGIDFTLTITSVQAEDLAVYYCQNDYRYPLTFGAGTKLELK

SEQ ID NO: 15 - protein, 118 aa, synthetic construct
  source 1..118: note = VH of Ab-AHZ
QVQLVQSGAEVKKPGASVKVSCKVSGSTFTDHTIHWMRQAPGQGLEWMGYIYPTSGSTQYAQKFQGRVTLTADRSTNTAYMELSSLRSEDTAVYFCSLRPLFNVIKHWGQGTLVTVSS

SEQ ID NO: 16 - protein, 113 aa, synthetic construct
  source 1..113: note = VL of Ab-AHZ
DIVMTQSPDSLAVSLGERATINCKSSQSLLDRGNHKNFLTWYQQKPGQPPKLLIFWASTRETGVPDRFSGSGSGIDFTLTISSLQAEDVAVYYCQNDYRYPLTFGQGTKLEIK

SEQ ID NO: 17 - protein, 7 aa, synthetic construct
  source 1..7: note = Ab-B/Ab-BHZ Chothia CDR-H1
GFTFTSY

SEQ ID NO: 18 - protein, 10 aa, synthetic construct
  source 1..10: note = Ab-B/Ab-BHZ AbM CDR-H1
GFTFTSYWMH

SEQ ID NO: 19 - protein, 5 aa, synthetic construct
  source 1..5: note = Ab-B/Ab-BHZ Kabat CDR-H1
SYWMH

SEQ ID NO: 20 - protein, 6 aa, synthetic construct
  source 1..6: note = Ab-B/Ab-BHZ Contact CDR-H1
TSYWMH

SEQ ID NO: 21 - protein, 8 aa, synthetic construct
  source 1..8: note = Ab-B/Ab-BHZ IMGT CDR-H1
GFTFTSYW

SEQ ID NO: 22 - protein, 6 aa, synthetic construct
  source 1..6: note = Ab-B Chothia CDR-H2
NLSYGN

SEQ ID NO: 23 - protein, 10 aa, synthetic construct
  source 1..10: note = Ab-B AbM CDR-H2
NINLSYGNTN

SEQ ID NO: 24 - protein, 17 aa, synthetic construct
  source 1..17: note = Ab-B Kabat CDR-H2
NINLSYGNTNYNEKFKN

SEQ ID NO: 25 - protein, 13 aa, synthetic construct
  source 1..13: note = Ab-B Contact CDR-H2
WIGNINLSYGNTN

SEQ ID NO: 26 - protein, 8 aa, synthetic construct
  source 1..8: note = Ab-B IMGT CDR-H2
INLSYGNT

SEQ ID NO: 27 - protein, 6 aa, synthetic construct
  source 1..6: note = Ab-BHZ Chothia CDR-H2
QLSYGN

SEQ ID NO: 28 - protein, 10 aa, synthetic construct
  source 1..10: note = Ab-BHZ AbM CDR-H2
NIQLSYGNTN

SEQ ID NO: 29 - protein, 17 aa, synthetic construct
  source 1..17: note = Ab-BHZ Kabat CDR-H2
NIQLSYGNTNYAQKFQG

SEQ ID NO: 30 - protein, 13 aa, synthetic construct
  source 1..13: note = Ab-BHZ Contact CDR-H2
WIGNIQLSYGNTN

SEQ ID NO: 31 - protein, 8 aa, synthetic construct
  source 1..8: note = Ab-BHZ IMGT CDR-H2
IQLSYGNT

SEQ ID NO: 32 - protein, 7 aa, synthetic construct
  source 1..7: note = Ab-B/Ab-BHZ Chothia/AbM/Kabat CDR-H3
GFSYFDY

SEQ ID NO: 33 - protein, 8 aa, synthetic construct
  source 1..8: note = Ab-B/Ab-BHZ Contact CDR-H3
ARGFSYFD

SEQ ID NO: 34 - protein, 9 aa, synthetic construct
  source 1..9: note = Ab-B/Ab-BHZ IMGT CDR-H3
ARGFSYFDY

SEQ ID NO: 35 - protein, 11 aa, synthetic construct
  source 1..11: note = Ab-B/Ab-BHZ Chothia/AbM/Kabat CDR-L1
KASQNVDTDVS

SEQ ID NO: 36 - protein, 7 aa, synthetic construct
  source 1..7: note = Ab-B/Ab-BHZ Contact CDR-L1
DTDVSWY

SEQ ID NO: 37 - protein, 6 aa, synthetic construct
  source 1..6: note = Ab-B/Ab-BHZ IMGT CDR-L1
QNVDTD

SEQ ID NO: 38 - protein, 7 aa, synthetic construct
  source 1..7: note = Ab-B/Ab-BHZ Chothia/AbM/Kabat CDR-L2
WASNRFT

SEQ ID NO: 39 - protein, 10 aa, synthetic construct
  source 1..10: note = Ab-B Contact CDR-L2
PLIYWASNRF

SEQ ID NO: 40 - protein, 10 aa, synthetic construct
  source 1..10: note = Ab-BHZ Contact CDR-L2
SLIYWASNRF

SEQ ID NO: 41 - skipped

SEQ ID NO: 42 - protein, 9 aa, synthetic construct
  source 1..9: note = Ab-B/Ab-BHZ Chothia/AbM/Kabat/IMGT CDR-L3
EQYSSYPFT

SEQ ID NO: 43 - protein, 8 aa, synthetic construct
  source 1..8: note = Ab-B/Ab-BHZ Contact CDR-L3
EQYSSYPF

SEQ ID NO: 44 - protein, 7 aa, synthetic construct
  source 1..7: note = Ab-C/Ab-CHZ Chothia CDR-H1
GFTFTSF

SEQ ID NO: 45 - protein, 10 aa, synthetic construct
  source 1..10: note = Ab-C/Ab-CHZ AbM CDR-H1
GFTFTSFGMH

SEQ ID NO: 46 - protein, 5 aa, synthetic construct
  source 1..5: note = Ab-C/Ab-CHZ Kabat CDR-H1
SFGMH

SEQ ID NO: 47 - protein, 6 aa, synthetic construct
  source 1..6: note = Ab-C/Ab-CHZ Contact CDR-H1
TSFGMH

SEQ ID NO: 48 - protein, 8 aa, synthetic construct
  source 1..8: note = Ab-C/Ab-CHZ IMGT CDR-H1
GFTFTSFG

SEQ ID NO: 49 - protein, 6 aa, synthetic construct
  source 1..6: note = Ab-C/Ab-CHZ Chothia CDR-H2
SSVSNI

SEQ ID NO: 50 - protein, 10 aa, synthetic construct
  source 1..10: note = Ab-C/Ab-CHZ AbM CDR-H2
YISSVSNIIY

SEQ ID NO: 51 - protein, 17 aa, synthetic construct
  source 1..17: note = Ab-C Kabat CDR-H2
YISSVSNIIYYADTVKG

SEQ ID NO: 52 - protein, 17 aa, synthetic construct
  source 1..17: note = Ab-CHZ Kabat CDR-H2
YISSVSNIIYYADSVKG

SEQ ID NO: 53 - protein, 13 aa, synthetic construct
  source 1..13: note = Ab-C/Ab-CHZ Contact CDR-H2
WVAYISSVSNIIY

SEQ ID NO: 54 - protein, 8 aa, synthetic construct
  source 1..8: note = Ab-C/Ab-CHZ IMGT CDR-H2
ISSVSNII

SEQ ID NO: 55 - protein, 11 aa, synthetic construct
  source 1..11: note = Ab-C/Ab-CHZ Chothia/AbM/Kabat CDR-H3
DGKYYYYTMDY

SEQ ID NO: 56 - protein, 12 aa, synthetic construct
  source 1..12: note = Ab-C/Ab-CHZ Contact CDR-H3
ARDGKYYYYTMD

SEQ ID NO: 57 - protein, 13 aa, synthetic construct
  source 1..13: note = Ab-C/Ab-CHZ IMGT CDR-H3
ARDGKYYYYTMDY

SEQ ID NO: 58 - protein, 14 aa, synthetic construct
  source 1..14: note = Ab-C/Ab-CHZ Chothia/AbM/Kabat CDR-L1
RSSTGAVTTTNYAN

SEQ ID NO: 59 - protein, 10 aa, synthetic construct
  source 1..10: note = Ab-C/Ab-CHZ Contact CDR-L1
VTTTNYANWV

SEQ ID NO: 60 - protein, 9 aa, synthetic construct
  source 1..9: note = Ab-C/Ab-CHZ IMGT CDR-L1
TGAVTTTNY

SEQ ID NO: 61 - protein, 7 aa, synthetic construct
  source 1..7: note = Ab-C Chothia/AbM/Kabat CDR-L2
GTNNRTP

SEQ ID NO: 62 - protein, 10 aa, synthetic construct
  source 1..10: note = Ab-C Contact CDR-L2
GLIGGTNNRT

SEQ ID NO: 63 - protein, 7 aa, synthetic construct
  source 1..7: note = Ab-CHZ Chothia/AbM/Kabat CDR-L2
GTNQRTP

SEQ ID NO: 64 - protein, 10 aa, synthetic construct
  source 1..10: note = Ab-CHZ Contact CDR-L2
ALIGGTNQRT

SEQ ID NO: 65 - skipped

SEQ ID NO: 66 - protein, 9 aa, synthetic construct
  source 1..9: note = Ab-C/Ab-CHZ Chothia/AbM/Kabat/IMGT CDR-L3
ALWYSNHWV

SEQ ID NO: 67 - protein, 8 aa, synthetic construct
  source 1..8: note = Ab-C/Ab-CHZ Contact CDR-L3
ALWYSNHW

SEQ ID NO: 68 - protein, 7 aa, synthetic construct
  source 1..7: note = Ab-D/Ab-DHZ Chothia CDR-H1
GYTFSSY

SEQ ID NO: 69 - protein, 10 aa, synthetic construct
  source 1..10: note = Ab-D/Ab-DHZ AbM CDR-H1
GYTFSSYWIE

SEQ ID NO: 70 - protein, 5 aa, synthetic construct
  source 1..5: note = Ab-D/Ab-DHZ Kabat CDR-H1
SYWIE

SEQ ID NO: 71 - protein, 6 aa, synthetic construct
  source 1..6: note = Ab-D/Ab-DHZ Contact CDR-H1
SSYWIE

SEQ ID NO: 72 - protein, 8 aa, synthetic construct
  source 1..8: note = Ab-D/Ab-DHZ IMGT CDR-H1
GYTFSSYW

SEQ ID NO: 73 - protein, 6 aa, synthetic construct
  source 1..6: note = Ab-D/Ab-DHZ Chothia CDR-H2
LPGSGN

SEQ ID NO: 74 - protein, 10 aa, synthetic construct
  source 1..10: note = Ab-D/Ab-DHZ AbM CDR-H2
EILPGSGNTN

SEQ ID NO: 75 - protein, 17 aa, synthetic construct
  source 1..17: note = Ab-D Kabat CDR-H2
EILPGSGNTNYNEKFKG

SEQ ID NO: 76 - protein, 13 aa, synthetic construct
  source 1..13: note = Ab-D Contact CDR-H2
RIGEILPGSGNTN

SEQ ID NO: 77 - protein, 17 aa, synthetic construct
  source 1..17: note = Ab-DHZ Kabat CDR-H2
EILPGSGNTNYAQKFQG

SEQ ID NO: 78 - protein, 13 aa, synthetic construct
  source 1..13: note = Ab-DHZ Contact CDR-H2
RMGEILPGSGNTN

SEQ ID NO: 79 - protein, 8 aa, synthetic construct
  source 1..8: note = Ab-D/Ab-DHZ IMGT CDR-H2
ILPGSGNT

SEQ ID NO: 80 - protein, 9 aa, synthetic construct
  source 1..9: note = Ab-D/Ab-DHZ Chothia/AbM/Kabat CDR-H3
REYFGSRDY

SEQ ID NO: 81 - protein, 10 aa, synthetic construct
  source 1..10: note = Ab-D/Ab-DHZ Contact CDR-H3
ARREYFGSRD

SEQ ID NO: 82 - protein, 11 aa, synthetic construct
  source 1..11: note = Ab-D/Ab-DHZ IMGT CDR-H3
ARREYFGSRDY

SEQ ID NO: 83 - protein, 11 aa, synthetic construct
  source 1..11: note = Ab-D Chothia/AbM/Kabat CDR-L1
RASQDISNYLN

SEQ ID NO: 84 - protein, 11 aa, synthetic construct
  source 1..11: note = Ab-DHZ Chothia/AbM/Kabat CDR-L1
QASQDISNYLN

SEQ ID NO: 85 - protein, 7 aa, synthetic construct
  source 1..7: note = Ab-D/Ab-DHZ Contact CDR-L1
SNYLNWY

SEQ ID NO: 86 - protein, 6 aa, synthetic construct
  source 1..6: note = Ab-D/Ab-DHZ IMGT CDR-L1
QDISNY

SEQ ID NO: 87 - protein, 7 aa, synthetic construct
  source 1..7: note = Ab-D Chothia/AbM/Kabat CDR-L2
YTSRLHS

SEQ ID NO: 88 - protein, 7 aa, synthetic construct
  source 1..7: note = Ab-DHZ Chothia/AbM/Kabat CDR-L2
YTSRLHT

SEQ ID NO: 89 - protein, 10 aa, synthetic construct
  source 1..10: note = Ab-D/Ab-DHZ Contact CDR-L2
LLIYYTSRLH

SEQ ID NO: 90 - skipped

SEQ ID NO: 91 - protein, 9 aa, synthetic construct
  source 1..9: note = Ab-D/Ab-DHZ Chothia/AbM/Kabat/IMGT CDR-L3
QQGNTLPYA

SEQ ID NO: 92 - protein, 8 aa, synthetic construct
  source 1..8: note = Ab-D/Ab-DHZ Contact CDR-L3
QQGNTLPY

SEQ ID NO: 93 - protein, 7 aa, synthetic construct
  source 1..7: note = Ab-A/Ab-AHZ Chothia CDR-H1
GSTFTDH

SEQ ID NO: 94 - protein, 10 aa, synthetic construct
  source 1..10: note = Ab-A/Ab-AHZ AbM CDR-H1
GSTFTDHTIH

SEQ ID NO: 95 - protein, 5 aa, synthetic construct
  source 1..5: note = Ab-A/Ab-AHZ Kabat CDR-H1
DHTIH

SEQ ID NO: 96 - protein, 6 aa, synthetic construct
  source 1..6: note = Ab-A/Ab-AHZ Contact CDR-H1
TDHTIH

SEQ ID NO: 97 - protein, 8 aa, synthetic construct
  source 1..8: note = Ab-A/Ab-AHZ IMGT CDR-H1
GSTFTDHT

SEQ ID NO: 98 - protein, 6 aa, synthetic construct
  source 1..6: note = Ab-A Chothia CDR-H2
YPTDGS

SEQ ID NO: 99 - protein, 10 aa, synthetic construct
  source 1..10: note = Ab-A AbM CDR-H2
YIYPTDGSTQ

SEQ ID NO: 100 - protein, 17 aa, synthetic construct
  source 1..17: note = Ab-A Kabat CDR-H2
YIYPTDGSTQYNEKFRG

SEQ ID NO: 101 - protein, 13 aa, synthetic construct
  source 1..13: note = Ab-A Contact CDR-H2
WIGYIYPTDGSTQ

SEQ ID NO: 102 - protein, 8 aa, synthetic construct
  source 1..8: note = Ab-A IMGT CDR-H2
IYPTDGST

SEQ ID NO: 103 - protein, 6 aa, synthetic construct
  source 1..6: note = Ab-AHZ Chothia CDR-H2
YPTSGS

SEQ ID NO: 104 - protein, 10 aa, synthetic construct
  source 1..10: note = Ab-AHZ AbM CDR-H2
YIYPTSGSTQ

SEQ ID NO: 105 - protein, 17 aa, synthetic construct
  source 1..17: note = Ab-AHZ Kabat CDR-H2
YIYPTSGSTQYAQKFQG

SEQ ID NO: 106 - protein, 13 aa, synthetic construct
  source 1..13: note = Ab-AHZ Contact CDR-H2
WMGYIYPTSGSTQ

SEQ ID NO: 107 - protein, 8 aa, synthetic construct
  source 1..8: note = Ab-AHZ IMGT CDR-H2
IYPTSGST

SEQ ID NO: 108 - protein, 9 aa, synthetic construct
  source 1..9: note = Ab-A/Ab-AHZ Chothia/AbM/Kabat CDR-H3
RPLFNVIKH

SEQ ID NO: 109 - protein, 10 aa, synthetic construct
  source 1..10: note = Ab-A/Ab-AHZ Contact CDR-H3
SLRPLFNVIK

SEQ ID NO: 110 - protein, 11 aa, synthetic construct
  source 1..11: note = Ab-A/Ab-AHZ IMGT CDR-H3
SLRPLFNVIKH

SEQ ID NO: 111 - protein, 17 aa, synthetic construct
  source 1..17: note = Ab-A Chothia/AbM/Kabat CDR-L1
KSSQSLLDSGNHKNFLT

SEQ ID NO: 112 - protein, 13 aa, synthetic construct
  source 1..13: note = Ab-A Contact CDR-L1
LDSGNHKNFLTWY

SEQ ID NO: 113 - protein, 12 aa, synthetic construct
  source 1..12: note = Ab-A IMGT CDR-L1
QSLLDSGNHKNF

SEQ ID NO: 114 - protein, 17 aa, synthetic construct
  source 1..17: note = Ab-AHZ Chothia/AbM/Kabat CDR-L1
KSSQSLLDRGNHKNFLT

SEQ ID NO: 115 - protein, 13 aa, synthetic construct
  source 1..13: note = Ab-AHZ Contact CDR-L1
LDRGNHKNFLTWY

SEQ ID NO: 116 - protein, 12 aa, synthetic construct
  source 1..12: note = Ab-AHZ IMGT CDR-L1
QSLLDRGNHKNF

SEQ ID NO: 117 - protein, 7 aa, synthetic construct
  source 1..7: note = Ab-A/Ab-AHZ Chothia/AbM/Kabat CDR-L2
WASTRET

SEQ ID NO: 118 - protein, 10 aa, synthetic construct
  source 1..10: note = Ab-A/Ab-AHZ Contact CDR-L2
LLIFWASTRE

SEQ ID NO: 119 - protein, 9 aa, synthetic construct
  source 1..9: note = Ab-A/Ab-AHZ Chothia/AbM/Kabat/IMGT CDR-L3
QNDYRYPLT

SEQ ID NO: 120 - protein, 8 aa, synthetic construct
  source 1..8: note = Ab-A/Ab-AHZ Contact CDR-L3
QNDYRYPL

SEQ ID NO: 121 - protein, 330 aa, synthetic construct [sequence omitted]

SEQ ID NO: 122 - protein, 330 aa, synthetic construct
  source 1..330: note = CH of Ab-BHZm
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 107 aa, synthetic construct [sequence omitted]

SEQ ID NO: 124 - protein, 446 aa, synthetic construct
  source 1..446: note = HC of Ab-BHZ
QVQLVQSGAEVKKPGASVKVSCKASGFTFTSYWMHWVRQAPGQGLEWIGNIQLSYGNTNYAQKFQGRVTLTVDKSSSTAYMELSSLRSEDTAVYYCARGFSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 214 aa, synthetic construct
  source 1..214: note = LC of Ab-BHZ\Ab-BHZm
DIQMTQSPSSLSASVGDRVTITCKASQNVDTDVSWYQQKPGKAPKSLIYWASNRFTGVPSRFSGSGSGTDFTLTISSLQPEDFATYFCEQYSSYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 446 aa, synthetic construct
  source 1..446: note = HC of Ab-BHZm
QVQLVQSGAEVKKPGASVKVSCKASGFTFTSYWMHWVRQAPGQGLEWIGNIQLSYGNTNYAQKFQGRVTLTVDKSSSTAYMELSSLRSEDTAVYYCARGFSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 446 aa, synthetic construct
  source 1..446: note = HC of Ab-B
QVQLQQPGTELVKPGASVKLSCQASGFTFTSYWMHWVKQRPGQGLAWIGNINLSYGNTNYNEKFKNKATLTVDKSSSTAYMQLSGLTSEDSAVYYCARGFSYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 214 aa, synthetic construct
  source 1..214: note = LC of Ab-B
DIVMTQSHKFMSTSLGDRVNITCKASQNVDTDVSWYQQKPGKSPKPLIYWASNRFTGVPDRFTGSGSGTDFTLTINNVQSEDLADYFCEQYSSYPFTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 450 aa, synthetic construct
  source 1..450: note = HC of Ab-CHZ
EVQLVESGGGLVQPGGSLRLSCAASGFTFTSFGMHWVRQAPGKGLEWVAYISSVSNIIYYADSVKGRFTISRDNAKNSLYLQMNSLRAEDTAVYYCARDGKYYYYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 215 aa, synthetic construct
  source 1..215: note = LC of Ab-CHZ
QTVVTQEPSLTVSPGGTVTLTCRSSTGAVTTTNYANWVQQKPGQAFRALIGGTNQRTPWTPARFSGSLLGGKAALTLSGVQPEDEAEYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 131 - protein, 450 aa, synthetic construct
  source 1..450: note = HC of Ab-C
DVQLVESGGGLVQPGGSRKLSCAASGFTFTSFGMHWVRQTPEKGLEWVAYISSVSNIIYYADTVKGRFTISRDNPKNTLFLQMTSLRSEDTAMYYCARDGKYYYYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 215 aa, synthetic construct
  source 1..215: note = LC of Ab-C
QAVVTQESALTTSPGETVTLTCRSSTGAVTTTNYANWVQEKPDHLFTGLIGGTNNRTPGVPARFSGSLIGDKAALTITGAQTEDEAVYFCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 133 - protein, 448 aa, synthetic construct
  source 1..448: note = HC of Ab-DHZ
QVQLVQSGAEVKKPGSSVKVSCKASGYTFSSYWIEWVRQAPGQGLERMGEILPGSGNTNYAQKFQGRVTITADKSTNTAYMELSSLRSEDTAVYYCARREYFGSRDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 214 aa, synthetic construct
  source 1..214: note = LC of Ab-DHZ
DIQMTQSPSSLSASVGDRVTITCQASQDISNYLNWYQQKPEGAVKLLIYYTSRLHTGVPSRFSGSGSGTDYTFTISSLQPEDIATYYCQQGNTLPYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 448 aa, synthetic construct
  source 1..448: note = HC of Ab-D
QVQLQQSGAELMKPGASVKISCKATGYTFSSYWIEWVKQRPGHGLERIGEILPGSGNTNYNEKFKGKATFTADTSSNTAYMQLSSLTAEDSAVYYCARREYFGSRDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 214 aa, synthetic construct
  source 1..214: note = LC of Ab-D
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 448 aa, synthetic construct
  source 1..448: note = HC of Ab-AHZ
QVQLVQSGAEVKKPGASVKVSCKVSGSTFTDHTIHWMRQAPGQGLEWMGYIYPTSGSTQYAQKFQGRVTLTADRSTNTAYMELSSLRSEDTAVYFCSLRPLFNVIKHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 220 aa, synthetic construct
  source 1..220: note = LC of Ab-AHZ
DIVMTQSPDSLAVSLGERATINCKSSQSLLDRGNHKNFLTWYQQKPGQPPKLLIFWASTRETGVPDRFSGSGSGIDFTLTISSLQAEDVAVYYCQNDYRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 448 aa, synthetic construct
  source 1..448: note = HC of Ab-A
QVQLQQSDAELVKPGVSVKISCKVSGSTFTDHTIHWMKQRPEQGLEWIGYIYPTDGSTQYNEKFRGKATLTADRSSNTAYMQLNSLTYEDSAVYFSSLRPLFNVIKHWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 220 aa, synthetic construct
  source 1..220: note = LC of Ab-A
DIVMTQSPSSLTVTAGEKVTMSCKSSQSLLDSGNHKNFLTWYQQKPGQPPKLLIFWASTRETGVPDRFTGSGSGIDFTLTITSVQAEDLAVYYCQNDYRYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 446 aa, synthetic construct
  source 1..446: note = Ab-BHZm heavy chain amino acid sequence (N-terminal pyroglutamylation)
  MOD_RES 1: note = X is pyroglutamic acid or pyroglutamate
XVQLVQSGAEVKKPGASVKVSCKASGFTFTSYWMHWVRQAPGQGLEWIGNIQLSYGNTNYAQKFQGRVTLTVDKSSSTAYMELSSLRSEDTAVYYCARGFSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 445 aa, synthetic construct
  source 1..445: note = Ab-BHZm heavy chain amino acid sequence (N-terminal pyroglutamylation; deletion of C-terminal lysine)
  MOD_RES 1: note = X is pyroglutamic acid or pyroglutamate
XVQLVQSGAEVKKPGASVKVSCKASGFTFTSYWMHWVRQAPGQGLEWIGNIQLSYGNTNYAQKFQGRVTLTVDKSSSTAYMELSSLRSEDTAVYYCARGFSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 445 aa, synthetic construct
  source 1..445: note = Ab-BHZm heavy chain amino acid sequence (deletion of C-terminal lysine)
QVQLVQSGAEVKKPGASVKVSCKASGFTFTSYWMHWVRQAPGQGLEWIGNIQLSYGNTNYAQKFQGRVTLTVDKSSSTAYMELSSLRSEDTAVYYCARGFSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 106 aa, synthetic construct
  source 1..106: note = Lambda CL
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 145 - protein, 4 aa, synthetic construct
  source 1..4: note = Short chain polypeptide
GGFG

SEQ ID NO: 146 - protein, 4 aa, synthetic construct
  source 1..4: note = Short chain polypeptide
GGVA

SEQ ID NO: 147 - protein, 4 aa, synthetic construct
  source 1..4: note = Short chain polypeptide
GFLG

SEQ ID NO: 148 - protein, 4 aa, synthetic construct
  source 1..4: note = Short chain polypeptide
EAAA

SEQ ID NO: 149 - protein, 5 aa, synthetic construct
  source 1..5: note = Short chain polypeptide
GGGGG

SEQ ID NO: 150 - protein, 5 aa, synthetic construct
  source 1..5: note = Linker subunit
GGGGS

SEQ ID NO: 151 - protein, 20 aa, synthetic construct
  source 1..20: note = (GGGGS)4
GGGGSGGGGSGGGGSGGGGS